SEQUENCE LISTING (304 sequences)

SEQ ID NO: 1 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 2 - protein, 567 aa, Anabaena variabilis; identical to SEQ ID NO: 1

SEQ ID NO: 3 - protein, 532 aa, Photorhabdus luminescens
MKAKDVQPTIIINKNGLISLEDIYDIAIKQKKVEISTEITELLTHGREKLEEKLNSGEVIYGINTGFGGNANLVVPFEKIAEHQQNLLTFLSAGTGDYMSKPCIKASQFTMLLSVCKGWSATRPIVAQAIVDHINHDIVPLVPRYGSVGASGDLIPLSYIARALCGIGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTLATEQYNQDIVSLGLHAAQDVLEMEQKLRNIVSMTILVVCQAIHLRGNISEIAPETAKFYHAVREISSPLITDRALDEDIIRIADAIINDQLPLPEIMLEE

SEQ ID NO: 4 - protein, 514 aa, Photorhabdus luminescens
MKQLTIYPGKLTLDELRQVYLQPVKITLDSQIFPAIERSVECVNAILAENRTAYGINTGFGLLASTRIEEDNLEKLQRSLVVSHAAGVGKALDDNMTRLIMVLKINSLSRGYSGIRLAVIQALIALVNAEIYPHIPCKGSVGASGDLAPLAHMSLLLLGEGQARYQGEWLPAKEALAKANLQPITLAAKEGLALLNGTQVSTAFALRGLFEAEDLLAAAIVCGSLSVEAALGSRKPFDARVHVVRGQQGQIDVAALYRHVLEESSELSDSHINCPKVQDPYSLRCQPQVMGACLTQLRHAADVILTEANAVSDNPLVFAEQGEVISGGNFHAEPVAMASDNLALVLAEIGALSERRIALLMDSHMSQLPPFLVENGGVNSGFMIAQVTAAALASENKALAHPASVDSLPTSANQEDHVSMAPAAGRRLWEMAENTRGILAIEWLSACQGIDFRNGLKSSPILEEARVILRAKVDYYDQDRFFAPDIDAAVKLLAEQHLSSLLPSGQILQRKNNR

SEQ ID NO: 5 - protein, 471 aa, Proteus mirabilis
MAISRRKFILGGTVVAVAAGAGVLTPMLTREGRFVPGTPRHGFVEGTGGPLPKQDDVVVIGAGILGIMTAINLAERGLSVTIVEKGNIAGEQSSRFYGQAISYKMPDETFLLHHLGKHRWREMNAKVGIDTTYRTQGRVEVPLDEEDLENVRKWIDAKSKDVGSDIPFRTKMIEGAELKQRLRGATTDWKIAGFEEDSGSFDPEVATFVMAEYAKKMGIKIFTNCAARGLETQAGVISDVVTEKGPIKTSRVVVAGGVGSRLFMQNLNVDVPTLPAYQSQQLISAAPNAPGGNVALPGGIFFRDQADGTYATSPRVIVAPVVKESFTYGYKYLPLLALPDFPVHISLNEQLINSFMQSTHWDLNEESPFEKYRDMTALPDLPELNASLEKLKKEFPAFKESTLIDQWSGAMAIAPDENPIISDVKEYPGLVINTATGWGMTESPVSAEITADLLLGKKPVLDAKPFSLYRF

SEQ ID NO: 6 - protein, 473 aa, Proteus mirabilis
MNISRRKLLLGVGAAGVLAGGAALVPMVRRDGKFVEAKSRASFVEGTQGALPKEADVVIIGAGIQGIMTAINLAERGMSVTILEKGQIAGEQSGRAYSQIISYQTSPEIFPLHHYGKILWRGMNEKIGADTSYRTQGRVEALADEKALDKAQAWIKTAKEAAGFDTPLNTRIIKGEELSNRLVGAQTPWTVAAFEEDSGSVDPETGTPALARYAKQIGVKIYTNCAVRGIETAGGKISDVVSEKGAIKTSQVVLAGGIWSRLFMGNMGIDIPTLNVYLSQQRVSGVPGAPRGNVHLPNGIHFREQADGTYAVAPRIFTSSIVKDSFLLGPKFMHLLGGGELPLEFSIGEDLFNSFKMPTSWNLDEKTPFEQFRVATATQNTQHLDAVFQRMKTEFPVFEKSEVVERWGAVVSPTFDELPIISEVKEYPGLVINTATVWGMTEGPAAGEVTADIVMGKKPVIDPTPFSLDRFKK

SEQ ID NO: 7 - protein, 471 aa, Proteus vulgaris
MAISRRKFIIGGTVVAVAAGAGILTPMLTREGRFVPGTPRHGFVEGTEGALPKQADVVVVGAGILGIMTAINLVERGLSVVIVEKGNIAGEQSSRFYGQAISYKMPDETFLLHHLGKHRWREMNAKVGIDTTYRTQGRVEVPLDEEDLVNVRKWIDERSKNVGSDIPFKTRIIEGAELNQRLRGATTDWKIAGFEEDSGSFDPEVATFVMAEYAKKMGVRIYTQCAARGLETQAGVISDVVTEKGAIKTSQVVVAGGVWSRLFMQNLNVDVPTLPAYQSQQLISGSPTAPGGNVALPGGIFFREQADGTYATSPRVIVAPVVKESFTYGYKYLPLLALPDFPVHISLNEQLINSFMQSTHWNLDEVSPFEQFRNMTALPDLPELNASLEKLKAEFPAFKESKLIDQWSGAMAIAPDENPIISEVKEYPGLVINTATGWGMTESPVSAELTADLLLGKKPVLDPKPFSLYRF

SEQ ID NO: 8 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEGKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 9 - DNA, 433 bp, Escherichia coli
  source 1..433: mol_type = genomic DNA
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 10 - DNA, 290 bp, Escherichia coli
  source 1..290: mol_type = genomic DNA
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 11 - DNA, 173 bp, Escherichia coli
  source 1..173: mol_type = genomic DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 12 - DNA, 305 bp, Escherichia coli
  source 1..305: mol_type = genomic DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 13 - DNA, 180 bp, Escherichia coli
  source 1..180: mol_type = genomic DNA
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 14 - DNA, 199 bp, Escherichia coli
  source 1..199: mol_type = genomic DNA
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 15 - DNA, 207 bp, Escherichia coli
  source 1..207: mol_type = genomic DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 16 - DNA, 390 bp, Escherichia coli
  source 1..390: mol_type = genomic DNA
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 17 - DNA, 200 bp, Escherichia coli
  source 1..200: mol_type = genomic DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 18 - DNA, 355 bp, synthetic construct
  misc_feature 1..355: note = Synthetic polynucleotide
  source 1..355: mol_type = other DNA
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 19 - DNA, 228 bp, synthetic construct
  misc_feature 1..228: note = Synthetic polynucleotide
  source 1..228: mol_type = other DNA
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 20 - DNA, 334 bp, synthetic construct
  misc_feature 1..334: note = Synthetic polynucleotide
  source 1..334: mol_type = other DNA
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 21 - DNA, 134 bp, synthetic construct
  misc_feature 1..134: note = Synthetic polynucleotide
  source 1..134: mol_type = other DNA
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 22 - DNA, 4428 bp, synthetic construct
  misc_feature 1..4428: note = Synthetic polynucleotide
  source 1..4428: mol_type = other DNA
ctcttgatcgttatcaattcccacgctgtttcagagcgttaccttgcccttaaacattagcaatgtcgatttatcagagggccgacaggctcccacaggagaaaaccgatgaaaacactatcacaggcccaatctaaaacttcttcacagcaattcagctttaccgggaactcgtctgcgaatgtaattatcggcaatcaaaagctgaccattaatgatgtagctcgcgttgcccggaatggcactttggtgtcactgacgaacaataccgacattctgcaaggtattcaagctagctgcgattatatcaataacgccgttgaatctggcgagccaatctacggggtaacaagcggttttggtgggatggcgaacgttgccattagccgtgaacaggcgagcgaacttcagaccaacctcgtttggttcctaaagacaggagctggtaataagttacctctggctgacgtaagagccgcgatgctgcttcgcgctaatagtcacatgcgcggcgccagtggtatccgtcttgagcttatcaagaggatggaaatcttcctcaacgcgggtgtcacaccatatgtttatgagtttggtagtatcggagccagtggtgatcttgttcccctgagttatattacgggttcattgattggtttagacccgtcctttaaagtggattttaacgggaaagaaatggacgccccgaccgctttacgacagcttaatctgagcccacttactttgctccctaaagaaggtcttgccatgatgaatggcacctctgtgatgactggaattgccgcgaattgtgtgtatgacacgcagatcctaacggccattgccatgggtgttcacgcgttggacattcaagccctgaatggtacaaaccagtcgtttcatccgtttatccataattcaaaaccccatccgggacagctttgggctgctgatcagatgatctcactcctggccaatagtcaactggttcgggacgagctcgacggcaaacatgattatcgcgatcatgagctcatccaggaccggtattcacttcgttgtctcccacaatacctggggcctatcgttgatggtatatctcaaattgcgaagcaaattgaaattgagatcaatagcgtaaccgacaacccgcttatcgatgttgataatcaggcctcttatcacggtggcaattttctgggccagtatgttggtatggggatggatcacctgcggtactatattgggcttctggctaaacatcttgatgtgcagattgccttattagcttcaccagaattttcaaatggactgccgccatcattgctcggtaacagagaaaggaaagtaaatatgggccttaagggccttcagatatgtggtaactcaatcatgcccctcctgaccttttatgggaactcaattgctgatcgttttccgacacatgctgaacagtttaaccaaaacattaactcacagggctatacatccgcgacgttagcgcgtcggtccgtggatatcttccagaattatgttgctatcgctctgatgttcggcgtacaggccgttgatttgcgcacttataaaaaaaccggtcactacgatgctcgggcttgcctgtcgcctgccaccgagcggctttatagcgccgtacgtcatgttgtgggtcagaaaccgacgtcggaccgcccctatatttggaatgataatgaacaagggctggatgaacacatcgcccggatatctgccgatattgccgccggaggtgtcatcgtccaggcggtacaagacatacttccttgcctgcattaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgtt

SEQ ID NO: 23 - DNA, 305 bp, Escherichia coli
  source 1..305: mol_type = genomic DNA
cagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacat

SEQ ID NO: 24 - DNA, 897 bp, Escherichia coli
  source 1..897: mol_type = genomic DNA
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcat

SEQ ID NO: 25 - protein, 298 aa, Escherichia coli
MQYGQLVSSLNGGSMKSMAEAQNDPLLPGYSFNAHLVAGLTPIEANGYLDFFIDRPLGMKGYILNLTIRGQGVVKNQGREFVCRPGDILLFPPGEIHHYGRHPEAHEWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCE

SEQ ID NO: 26 - DNA, 280 bp, Escherichia coli
  source 1..280: mol_type = genomic DNA
cggtgagcatcacatcaccacaattcagcaaattgtgaacatcatcacgttcatctttccctggttgccaatggcccattttcctgtcagtaacgagaaggtcgcgaatcaggcgctttttagactggtcgtaatgaaattcagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 27 - DNA, 326 bp, Escherichia coli
  source 1..326: mol_type = genomic DNA
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 28 - DNA, 1083 bp, Escherichia coli
  source 1..1083: mol_type = genomic DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 29 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 30 - DNA, 222 bp, Escherichia coli
  source 1..222: mol_type = genomic DNA
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 31 - DNA, 714 bp, synthetic construct
  misc_feature 1..714: note = Synthetic polynucleotide
  source 1..714: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 32 - DNA, 43 bp, Escherichia coli
  source 1..43: mol_type = genomic DNA
ctctagaaataattttgtttaactttaagaaggagatatacat

SEQ ID NO: 33 - protein, 237 aa, synthetic construct
  REGION 1..237: note = Synthetic polypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 34 - DNA, 748 bp, synthetic construct
  misc_feature 1..748: note = Synthetic polynucleotide
  source 1..748: mol_type = other DNA
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacat

SEQ ID NO: 35 - DNA, 225 bp, Escherichia coli
  source 1..225: mol_type = genomic DNA
tcacctttcccggattaaacgcttttttgcccggtggcatggtgctaccggcgatcacaaacggttaattatgacacaaattgacctgaatgaatatacagtattggaatgcattacccggagtgttgtgtaacaatgtctggccaggtttgtttcccggaaccgaggtcacaacatagtaaaagcgctattggtaatggtacaatcgcgcgtttacacttattc

SEQ ID NO: 36-63 - skipped (28 entries)

SEQ ID NO: 64 - DNA, 1955 bp, synthetic construct
  misc_feature 1..1955: note = Synthetic polynucleotide
  source 1..1955: mol_type = other DNA
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacatatgatcccggaaaagcgaattatacggcgcattcagtctggcggttgtgctatccattgccaggattgctatatcagccagctttgcatcccgttcacactcaacgaacatgagcttgatcagcttgataatatcattgagcggaagaagcctattcagaaaggccagacgctgtttaaggctggagatgaacttaaatcgctttatgccatccgctccggtacgattaaaagttataccatcactgagcaaggcgacgagcaaatcactggtttccatttagcaggcgatctggtgggatttgatgccatcggcagcggtcatcacccgagtttcgcgcaggcgctggaaacctcgatggtatgtgaaatcccgttcgaaacgctggacgatttgtctggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggtgatatcggtaactatctgggcctgacggttgaaaccatcagccgtctgctgggtcgcttccagaaaagcggtatgctggcagtcaaaggtaaatacatcactatcgaaaataacgatgcgctggcccagcttgctggtcatacgcgtaacgttgcctga

SEQ ID NO: 65 - DNA, 753 bp, synthetic construct
  misc_feature 1..753: note = Synthetic polynucleotide
  source 1..753: mol_type = other DNA
atgatcccggaaaagcgaattatacggcgcattcagtctggcggttgtgctatccattgccaggattgctatatcagccagctttgcatcccgttcacactcaacgaacatgagcttgatcagcttgataatatcattgagcggaagaagcctattcagaaaggccagacgctgtttaaggctggagatgaacttaaatcgctttatgccatccgctccggtacgattaaaagttataccatcactgagcaaggcgacgagcaaatcactggtttccatttagcaggcgatctggtgggatttgatgccatcggcagcggtcatcacccgagtttcgcgcaggcgctggaaacctcgatggtatgtgaaatcccgttcgaaacgctggacgatttgtctggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggtgatatcggtaactatctgggcctgacggttgaaaccatcagccgtctgctgggtcgcttccagaaaagcggtatgctggcagtcaaaggtaaatacatcactatcgaaaataacgatgcgctggcccagcttgctggtcatacgcgtaacgttgcctga

SEQ ID NO: 66 - DNA, 897 bp, Escherichia coli; identical to SEQ ID NO: 24
  source 1..897: mol_type = genomic DNA

SEQ ID NO: 67 - DNA, 305 bp, Escherichia coli; identical to SEQ ID NO: 23
  source 1..305: mol_type = genomic DNA

SEQ ID NO: 68 - DNA, 43 bp, Escherichia coli; identical to SEQ ID NO: 32
  source 1..43: mol_type = genomic DNA

SEQ ID NO: 69 - protein, 250 aa, synthetic construct
  REGION 1..250: note = Synthetic polypeptide
MIPEKRIIRRIQSGGCAIHCQDCYISQLCIPFTLNEHELDQLDNIIERKKPIQKGQTLFKAGDELKSLYAIRSGTIKSYTITEQGDEQITGFHLAGDLVGFDAIGSGHHPSFAQALETSMVCEIPFETLDDLSGKMPNLRQQMMRLMSGEIKGDQDMILLLSKKNAEERLAAFIYNLSRRFAQRGFSPREFRLTMTRGDIGNYLGLTVETISRLLGRFQKSGMLAVKGKYITIENNDALAQLAGHTRNVA

SEQ ID NO: 70 - protein, 298 aa, Escherichia coli; identical to SEQ ID NO: 25

SEQ ID NO: 71 - DNA, 753 bp, Escherichia coli
  source 1..753: mol_type = genomic DNA
atgatcccggaaaagcgaattatacggcgcattcagtctggcggttgtgctatccattgccaggattgcacgatcagccagctttgcatcccgttcacactcaacgaacatgagcttgatcagcttgataatatcattgagcggaagaagcctattcagaaaggccagacgctgtttaaggctggagatgaacttaaatcgctttatgccatccgctccggtacgattaaaagttataccatcactgagcaaggcgacgagcaaatcactggtttccatttagcaggcgatctggtgggatttgatgccatcggcagcggtcatcacccgagtttcgcgcaggcgctggaaacctcgatggtatgtgaaatcccgttcgaaacgctggacgatttgtctggtaaaatgccgaatctgcgtcagcagatgatgcgtctgatgagcggtgaaatcaaaggcgatcaggacatgatcctgctgttgtcgaagaaaaatgccgaggaacgtctggctgcattcatctacaacctgtcccgtcgttttgcccaacgcggcttctcccctcgtgaattccgcctgacgatgactcgtggtgatatcggtaactatctgggcctgacggttgaaaccatcagccgtctgctgggtcgcttccagaaaagcggtatgctggcagtcaaaggtaaatacatcactatcgaaaataacgatgcgctggcccagcttgctggtcatacgcgtaacgttgcctga

SEQ ID NO: 72 - protein, 250 aa, Escherichia coli
MIPEKRIIRRIQSGGCAIHCQDCSISQLCIPFTLNEHELDQLDNIIERKKPIQKGQTLFKAGDELKSLYAIRSGTIKSYTITEQGDEQITGFHLAGDLVGFDAIGSGHHPSFAQALETSMVCEIPFETLDDLSGKMPNLRQQMMRLMSGEIKGDQDMILLLSKKNAEERLAAFIYNLSRRFAQRGFSPREFRLTMTRGDIGNYLGLTVETISRLLGRFQKSGMLAVKGKYITIENNDALAQLAGHTRNVA

SEQ ID NO: 73-80 - skipped (8 entries)

SEQ ID NO: 81 - DNA, 2555 bp, synthetic construct
  misc_feature 1..2555: note = Synthetic polynucleotide
  source 1..2555: mol_type = other DNA
actcttcctttttcaatattattgaagcatttatcagggttattgtctcatgagcggatacatatttgaatgtatttagaaaaataaacaaataggggaattaaaaaaaagcccgctcattaggcgggctactacctaggccgcggccgcgcgaattcgagctcggtacccggggatcctctagagtcgacctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatcacagcaaacaccacgtcggccctatcagctgcgtgctttctatgagtcgttgctgcataacttgacaattaacatccggctcgtagggtttgtggagggcccaagttcacttaaaaaggagatcaacaatgaaagcaattttcgtactgaaacatcttaatcatgctggggagggtttctaatgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggccgtgagacggtcagagcggcgcttaaacatgccggtttgctgtaagacttttgtcaggttcctactgtgacgactaccaccgatagactggagtgttgctgcgaaaaaaccccgccgaagcggggttttttgcgagaagtcaccacgattgtgctttacacggagtagtcggcagttccttaagtcagaatagtggacaggcggccaagaacttcgttcatgatagtctccggaacccgttcgagtcgttttccgccccgtgctttcatatcaattgtccggggttgatcgcaacgtacaacacctgtggtacgtatgccaacaccatccaacgacaccgcaaagccggcagtgcgggcaaaattgcctccgctggttacgggcacaacaacaggcaggcgggtcacgcgattaaaggccgccggtgtgacaatcagcaccggccgcgttccctgctgctcatgacctgcggtaggatcaagcgagacaagccagatttcccctctttccatctagtataactattgtttctctagtaacatttattgtacaacacgagcccatttttgtcaaataaattttaaattatatcaacgttaataagacgttgtcaataaaattattttgacaaaattggccggccggcgcgccgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatggggcgcgcccagctgtctagggcggcggatttgtcctactcaggagagcgttcaccgacaaacaacagataaaacgaaaggcccagtctttcgactgagcctttcgttttatttgatgcct

SEQ ID NO: 82 - DNA, 2472 bp, synthetic construct
  misc_feature 1..2472: note = Synthetic polynucleotide
  source 1..2472: mol_type = other DNA
actcttcctttttcaatattattgaagcatttatcagggttattgtctcatgagcggatacatatttgaatgtatttagaaaaataaacaaataggggaattaaaaaaaagcccgctcattaggcgggctactacctaggccgcggccgcgcgaattcgagctcggtacccggggatcctctagagtcgacctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatcacagcaaacaccacgtcggccctatcagctgcgtgctttctatgagtcgttgctgcataacttgacaattaatcatccggctcgtagggtttgtggagggcccaagttcacttaaaaaggagatcaacaatgaaagcaattttcgtactgaaacatcttaatcatgctggggagggtttctaatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttcttcagggcgacactaacattgcttatctacacgaaaacaatgtcaccatctgggacgaatgggccgatgaaaacggcgacctcgggccagtgtatggtaaacagtggcgtgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaagacttttgtcaggttcctactgtgacgactaccaccgatagactggagtgttgctgcgaaaaaaccccgccgaagcggggttttttgcgagaagtcaccacgattgtgctttacacggagtagtcggcagttccttaagtcagaatagtggacaggcggccaagaacttcgttcatgatagtctccggaacccgttcgagtcgttttccgccccgtgctttcatatcaattgtccggggttgatcgcaacgtacaacacctgtggtacgtatgccaacaccatccaacgacaccgcaaagccggcagtgcgggcaaaattgcctccgctggttacgggcacaacaacaggcaggcgggtcacgcgattaaaggccgccggtgtgacaatcagcaccggccgcgttccctgctgctcatgacctgcggtaggatcaagcgagacaagccagatttcccctctttccatctagtataactattgtttctctagtaacatttattgtacaacacgagcccatttttgtcaaataaattttaaattatatcaacgttaataagacgttgtcaataaaattattttgacaaaattggccggccggcgcgccgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatggggcgcgcccagctgtctagggcggcggatttgtcctactcaggagagcgttcaccgacaaacaacagataaaacgaaaggcccagtctttcgactgagcctttcgttttatttgatgcct

SEQ ID NO: 83 - DNA, 333 bp, synthetic construct
  misc_feature 1..333: note = Synthetic polynucleotide
  source 1..333: mol_type = other DNA
ttaagtcagaatagtggacaggcggccaagaacttcgttcatgatagtctccggaacccgttcgagtcgttttccgccccgtgctttcatatcaattgtccggggttgatcgcaacgtacaacacctgtggtacgtatgccaacaccatccaacgacaccgcaaagccggcagtgcgggcaaaattgcctccgctggttacgggcacaacaacaggcaggcgggtcacgcgattaaaggccgccggtgtgacaatcagcaccggccgcgttccctgctgctcatgacctgcggtaggatcaagcgagacaagccagatttcccctctttccat

SEQ ID NO: 84 - DNA, 879 bp, synthetic construct
  misc_feature 1..879: note = Synthetic polynucleotide
  source 1..879: mol_type = other DNA
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggccgtgagacggtcagagcggcgcttaaacatgccggtttgctgtaa

SEQ ID NO: 85 - DNA, 795 bp, synthetic construct
  misc_feature 1..795: note = Synthetic polynucleotide
  source 1..795: mol_type = other DNA
atgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttcttcagggcgacactaacattgcttatctacacgaaaacaatgtcaccatctgggacgaatgggccgatgaaaacggcgacctcgggccagtgtatggtaaacagtggcgtgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaa

SEQ ID NO: 86 - protein, 110 aa, synthetic construct
  REGION 1..110: note = Synthetic polypeptide
MERGEIWLVSLDPTAGHEQQGTRPVLIVTPAAFNRVTRLPVVVPVTSGGNFARTAGFAVSLDGVGIRTTGVVRCDQPRTIDMKARGGKRLERVPETIMNEVLGRLSTILT

SEQ ID NO: 87 - protein, 292 aa, synthetic construct
  REGION 1..292: note = Synthetic polypeptide
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 88 - protein, 264 aa, synthetic construct
  REGION 1..264: note = Synthetic polypeptide
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 89-117 - skipped (29 entries)

SEQ ID NO: 118 - DNA, 20 bp, synthetic construct
  misc_feature 1..20: note = Synthetic primer
  source 1..20: mol_type = other DNA
agtgcctgtaccagacgttc

SEQ ID NO: 119 - DNA, 20 bp, synthetic construct
  misc_feature 1..20: note = Synthetic primer
  source 1..20: mol_type = other DNA
agaaatgacaaccagagagc

SEQ ID NO: 120 - DNA, 21 bp, synthetic construct
  misc_feature 1..21: note = Synthetic primer
  source 1..21: mol_type = other DNA
ttgagtttaatatggcagaac

SEQ ID NO: 121 - DNA, 19 bp, synthetic construct
  misc_feature 1..19: note = Synthetic primer
  source 1..19: mol_type = other DNA
aaatgatcatcgcgtcatc

SEQ ID NO: 122 - DNA, 19 bp, synthetic construct
  misc_feature 1..19: note = Synthetic primer
  source 1..19: mol_type = other DNA
gcatcaatcagtgattggc

SEQ ID NO: 123 - DNA, 20 bp, synthetic construct
  misc_feature 1..20: note = Synthetic primer
  source 1..20: mol_type = other DNA
acgtctgaatatacgggctg

SEQ ID NO: 124 - DNA, 20 bp, synthetic construct
  misc_feature 1..20: note = Synthetic primer
  source 1..20: mol_type = other DNA
tccagcttgactcgtttcag

SEQ ID NO: 125 - DNA, 19 bp, synthetic construct
  misc_feature 1..19: note = Synthetic primer
  source 1..19: mol_type = other DNA
agcaccttacccgaagagt

SEQ ID NO: 126 - DNA, 79 bp, synthetic construct
  misc_feature 1..79: note = Synthetic primer
  source 1..79: mol_type = other DNA
tggaggctttaagaaatacctcgatgtgaacaaccgcctgccacgaatcttcgtcaagcgattacacgtcttgagcgat

SEQ ID NO: 127 - DNA, 79 bp, synthetic construct
  misc_feature 1..79: note = Synthetic primer
  source 1..79: mol_type = other DNA
gataatggtgagattatccccggttataccggacttatcgcctattcagaatcactggatctgacatgggaattagcca

SEQ ID NO: 128 - DNA, 20 bp, synthetic construct
  misc_feature 1..20: note = Synthetic primer
  source 1..20: mol_type = other DNA
tggaggctttaagaaatacc

SEQ ID NO: 129 - DNA, 20 bp, synthetic construct
  misc_feature 1..20: note = Synthetic primer
  source 1..20: mol_type = other DNA
gctggcgattcaggttcatc

SEQ ID NO: 130 - DNA, 9687 bp, Escherichia coli
  source 1..9687: mol_type = genomic DNA
aatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtg

SEQ ID NO: 131 - skipped

SEQ ID NO: 132 - DNA, 19 bp, synthetic construct
  misc_feature 1..19: note = Synthetic primer
  source 1..19: mol_type = other DNA
gcatcaatcagtgattggc

SEQ ID NO: 133 - DNA, 20 bp, synthetic construct
  misc_feature 1..20: note = Synthetic primer
  source 1..20: mol_type = other DNA
acgtctgaatatacgggctg

SEQ ID NO: 134 - DNA, 59056 bp, Escherichia coli
  source 1..59056: mol_type = genomic DNA
aggcctctcctcgcgagaggcattttttatttgatgggataaagatctttgcgcttatacggttggatttcgcccggtttgcgagttttcagcaattttaatatccaggtgtattgttctggtcgcggaccaacaaaaatctcgacttcttcattcatccgccgcgcaatcgtatgatcatccgcctctaacagatcatccatcggtgggcgcacctgaatcgtcagacgatgcgtcttgccatcataaatcggaaatagcggtacaacgcgcgcacggcacactttcatcaaacgaccaatcgcgggcaacgtcgctttataggtggcaaagaaatcaacaaattcgctgtgttctgggccatgatcctgatcgggtaaataatatccccagtaaccctgacgtaccgactggatgaatggtttaataccatcatttctcgcatgcagacgaccaccaaagcgacggcgcaccgtgttccagacataatcaaaaaccgggttgccctgattatggaacatcgctgccattttctgcccttgcgaggccatcagcatggcaggaatatcgacggcccaaccgtgcggcaccagaaaaatcactttctcgttattacgtcgtatctcttcgatgatctccagcccttgccagtcaacgcgcggctgaattttctccggcccgcgtattgccaactcagccatcattaccatcgcttgcggcgcggtggcaaacatctcatctacaatcgcttcgcgttcagcttcactacgttctggaaagcagagcgacagattgattaacgcacgacggcgtgagctttttcccagtcgtccggcaaaacgtcccagccgtgccagaatgggatcacggaactttggcggcgttaaagcgatacccgccatcgctgctacgcccagccatgctccccagtagcgcgggtggcgaaaggatttatcaaactcaggaatgtattcgctattattttttttcgtttccatgcttttccagtttcggataaggcaaaaatcaatctggtgatagtgtagcggcgcaacttgccccgcaccaaataaaaaagccggtactgactgcgtaccggctgcgaatggatgttaattaatcaaaccgtagctgcggcacaatctctttggcctgtgccaggaattcgcgacgatcggagccggtcagcccttcggtacgcggcagttttgccgtcagcgggtttacggcctgctggtttatccatacttcatagtgcagatgcggcccggttgaacgtccggtattaccggaaagcgcgatacggtcgccacgtttcaccttctgtcccggtttcaccaggatcttgcgcaagtgcatataacgcgtggtgtagctgcgaccatgacgaatagccacataataacctgctgcgccactacgtttggcaaccaccacttcaccgtcacccactgaaagcactggcgtaccttgtggcatggcaaaatcaacacctctgtgtggcgcaacgcgaccggtcaccggattagtacgacgcgggttaaagttagatgagatacggaactgtttcgccgtcgggaatcgcaagaatcctttcgccagaccagtaccgttacgatcgtagaatttgccatcttcagcgcggattgcgtaataatctttaccttctgaacgcaaacgtacgcccagcagctggctttgctcacgtttaccatcaagcatttctcgtgacattaacaccgcaaattcatcgccttttttcagtttgcggaaatccatttgccactgcatggctttaatcactgcgctcacttcggcgctggttaaaccggcgtttctggcgctggcaacaaagcttcccccgacggtacctttcagcagattgttgacccactctccttgctgcatttcgctggtcattttaaaaccgttagcggcagtacggtcataggttcgggtttcacgacgagacacttcccaggtgaggcgctgcagttcgccgtccgcggttaatgtccaggagagttgttgaccgattttcaggttacgcaattctttgtcggcagcagccagttgggtgatatcacccatatcaataccatactgattgagaatgctgcttagcgtatcgccagtggaaacaacatattcatgcacgcccgcttcaccggcgattttgtcatccagttcgtcctggggaatggcttcatcttcttgtgcagcttgatcaatcggctcactggcttcaggtaagagcgaacgaatttcgttctgttccagctcaatggttttgacaattggcgtggcatcgcggtgataaacatagggccgccagacagcgacggccagagtaagaacggtgagcgaccccaacataacgcggtgtggtcgcggtaaattattaaacgccagggcgacagagcgggctatctgttgcacgtaatcacttcctcattaatctcctttcaggcagctcgcatactggttggctaattgattcaggaattctgaatagcttgttttacccagtttgatattcgtccccaggggatccaacgttcccatacgaacggatgtccctcgtgcgacgctctcaacgaccgctggcctgaactgtggctcagcaaaaacgcaggttgctttttgctcaaccaactgtgttcttatttcatgtaaacgctgcgcgccaggttgaatctcagggttaacggtaaaatgaccaagcggtgtcagtccgaactgtttttcgaaatagccgtaagcatcgtgaaaaacgaaataacctttccccttgagcggcgcgagctcgttaccaacctgcttttcggttgaggctaattgtgcctcaaaatccttcaggttggcgtcaagtttggctcgactttgcggcataagttccactaattttccatggattgcaaccgctgtagcccgcgctatctctggggaaagccaaagatgcatgttgaaatcgccgtgatggtgatcttcgtcacttttttccgcgtggtcgtgatcatcatcatcgccgtgaatacttttcatcagcagcggtttcacattctctagctgcgcaatcgttacctgtttcgcttcaggtaatttacttaccggtttttgcatgaacgcttccatctccgggccaacccaaacgactaagtccgcgttctgtaagcgttttacatctgatggacgcagtgaataatcatgttctgaagccccgtcaggtagtaaaacctccgtttctgttaccccatcagcaatggcagaagcgatgaacccaacgggtttaagcgaagcgacaacggcagcatctgcggcctgtgttgcaccgccccagagagcggcggataatgctgcgaaaagaagcgtttttttatgtaacataatgcgaccaatcatcgtaatgaatatgagaagtgtgatattataacatttcatgactactgcaagactaaaattaacatgacaagtctggtttccctggaaaatgtctcggtttcttttggccaacgccgcgtcctctctgatgtgtcgctggaacttaaacctggaaaaattttgactttacttgggccaaacggcgcaggtaagtcgacactggtacgggtagtgctcgggctggtaacacccgatgaaggggttatcaagcgcaacggaaaactgcgcatcggctatgtaccgcagaagctgtatctcgacaccacgttgccactgaccgtaaaccgttttttacgcttacgccctggcacacataaagaagatattttgcctgcactgaaacgtgtccaggccgggcatctgattaacgcaccgatgcaaaagctctcgggtggcgaaacgcagcgtgtactgttagcgcgagcattgttaaatcgaccgcaattattagtgctggatgaacccactcagggcgtggatgtgaatggtcaggtggcgttatatgaccttattgaccaactgcgtcgcgaactggattgtggcgttttaatggtatctcacgatctgcatctggtaatggcaaaaaccgatgaagtgctttgcctgaatcaccacatttgttgttccggcacaccggaagttgtttccctgcatccggagtttatttctatgtttggtcctcgtggtgctgaacaactgggtatctatcgccatcatcataatcatcgtcacgatttacagggacgaattgttttgcgtcggggaaatgatcgctcatgattgaattattatttcccggttggttagccgggatcatgctcgcctgtgccgcgggtccgctgggttcgtttgtagtctggcgtcgtatgtcttatttcggtgatacgctggctcatgcctcattacttggcgtcgcgtttggtttgttgctggacgtgaatccattctatgcggtgattgccgttacgctgctgctggcgggcggtctggtatggctggagaagcgtccacagctggcgatcgacacgttattagggattatggcgcacagtgccctgtcgctgggcctggtggtcgttagtctgatgtctaatattcgtgttgatttgatggcttacctgttcggtgatttactggcagtgacgccagaagatctcatctctattgcgattggcgtggtcatcgtggtggctattttgttctggcaatggcgcaatttgctgtcgatgacgattagcccggatctggcgtttgttgatggtgtgaaattacagcgcgtgaaattgttgttgatgctggtgacggcattgacgattggtgtagcgatgaaattcgtcggcgcgttgattattacttcactgctgattattcctgctgctactgcacgtcgctttgcccgcacgccggaacagatggctggtgtcgctgttttggtggggatggtggcagtgactggcggtttaaccttttccgcattttacgatacacctgcaggcccgtcggtggtgctatgcgcggcactgttatttattatcagtatgatgaaaaagcaggccagctaatctgtcgctgaacacatttgtcggatgcggcgcgagcgccttatcccacctgcggttcgctatctctggtaggcctgataagacgcgaacagcgtcgcatcaggcacactgccagtgtcggatgcggctcgagcgaccaatccgacttacggcatttctggcggcgtgatgccgaagtggttccacgcccgcactgtcgccatacgcccgcgcggtgtacgctgcaaaaagccttgctgaatcaaataaggttccagtacatcctcaatggtttcacgttcttcgccaatggctgccgccaggttatccagacctaccggcccaccaaagaacttatcgattaccgccagcaacaatttgcggtccatataatcgaaaccttcagcatcgacattcaacatatccagcgcctgagcagcgatatctgccgagatggtgccatcgtgcttcacttcagcgaaatcacgcactcgacgcagcagacggttggcaatacgtggcgtaccgcgcgcacgacgagcaacttccagcgcgccgtcatcactcatctcaagccccataaagcgtgcgctgcgactgacgatatattgcagatccggcacctgataaaactccagacgttgcacaataccaaaacgatcgcgcaacggtgatgtcagcgaacctgcgcgcgtggttgcaccaatcagggtaaacggcggcaaatcaattttaatggagcgtgccgccggaccttcaccaatcatgatatccagttggtaatcttccattgccggatacaacacctcttccaccactggtgaaagacggtggatctcatcaataaacagtacatcgtgtggttcaaggttagtgagcattgctgccagatcgcccgccttttccagcaccggaccagaagtcgtgcgtaaattaacgcccatttcattggcgacaatattggcaagcgtagttttacccaaccccggaggaccaaaaatcaatagatgatcgagggcatcgccgcgcagtttcgctgctttgatgaaaatctccatctgcgaacgaacctgcggctgaccaacatactcttccagtaatttagggcgaatggcgcgatctgccacatcttccggcaaagtggtaccggcagaaatcagacggtctgcttcaatcatcctttacctcataacgcggcgcgtagggcttcgcgaattaatgtttcactgctggcgtcagggcgagcgattttgctcaccatgcggcttgcttcttgtggtttatagcccagtgccaccagcgcagcaaccgcttcctgttcagcatcgtcggtcgccgggctggcaggagacgtgagtaccaggtcggcggctggcgtaaagagatcgccatgcaaacctttaaatcggtctttcatttcgacaatcaagcgttcggcggtttttttgccaatacccggcagtttcaccagtgcccccacttcttcacgctcaacggcattaacgaactgctgcgctgacattccggagaggatcgccagcgccaacttcgggccgacgccgttggttttgatcaactctttgaacaacgtgcgctcttgtttattgttaaaaccgtacagcagttgcgcgtcttcacgcaccacaaagtgggtgaaaacgatcgcttcctgacccgcttcagggagttcataaaaacaggtcatcggcatatgcacttcatagcctacgccgcccacttcaattaacaccagcgggggttgtttttcaatgatgatgcctctgagtctgcctatcacatgacgctcctgcgtaatgaatcaaagataatgctgtatgataaaaaaatgctggatagatatccagcgaaggatgaagaaaacttgcgaggtgtctcgatgatctgaaaaatggcgcagtataatttattctacagattatattggaagcaaatatttaaatattacatattcagcgaagaaatgtgtaataaaaatacacattgcgacccctgaaaaaaataaattttttatgctattacgtatattcatatctatttcaatggaatgacaacgtgaatattaattatcctgctgaatatgaaattggtgatatcgtctttacatgtataagtgctgccttatttggtcaaatatcagctgcatcaaattgctggagtaatcacgtcgggatcattatcggtcataacggtgaagactttctggttgcagaaagccgtgttcccctctcaaccatcactacgctatcccgttttattaaacgctctgctaatcaacgctatgctataaagcgattagacgccggactaacagaacaacaaaatcaacgaattgttgaacaggttccttcccggctacgcaaaatttaccacaccggttttaaatacgaatcttcgcgccagttctgttcaaaatttgtttttgatatttataaagaggcgctatgtattccggtgggtgaaatagagacgtttggagaattgttaaatagcaatccaaatgcaaaactcactttctggaaattctggttcttaggttctattccgtgggagcgtaaaaccgtcacgccagccagtttgtggcatcatccgggtttggtgttgattcacgcggtgggagttgaaacgcctcagcctgaactgaccgaggcggtataacttaacgcagtcgccctctcgccaggttcagtcgcgattcgctcatttgcatcgcattctgactaacgtggcagtgggtgatggcaatcgccagcgcatcggcggcatccgcctgtggattagcgggcagtttcagcaaggtgcggaccatatgctgcacctggcttttttcggcactaccaatacctaccactgtttgctttacctgacgtgccgcatattcaaataccggcaattcctgattcaccgccgccacaatcgccacgccgcgcgcctgccccagtttcagggctgagtcagcgttcttcgccataaagacctgttcaatggcgaaataatcaggctggaattgggtgatgatttccgtcacgcccgcatagatgagcttcagacgagacggtaaatcatccactttggtgcgtatgcatccgctacccaggtaggacagttgcctgcctacctggcggatgacgccatagccggtcacgcgcgaacccgggtcaatgccgagaataatagccatcacgcgtctccgttttgctgtttagcaggcctcatcagagagtcgctgcaacctcatcagagatttcaccgttatggtaaacttcctgcacgtcgtcgcaatcttccagcatatcgatcagacgcatcagtttcggtgcggtttctgcatccatatcagctttggtggacgggatcatggaaacttccgcgctgtctgctttcagacctgccgcttccagagcgtcgcgtactttgcccatttcttcccatgcagtgtagacatcaatcgcgccgtcatcataggtcacaacgtcttcagcaccggcttccagggctgcttccatgatggtgtcttcatcgcctttctcgaaggagatcacgccttttttgctgaacaaataagctacggaaccatcagtaccgaggttaccgccacatttgctgaatgcatgacgcacttcagcaacggtacggttgcggttgtcagacagacattcaatcatgattgccgtgccgccaggaccgtaaccttcgtagatgatggtttccatgtttgcatcatcatcaccgcccacacctcgtgcaattgcgcggttcagagtgtcacgggtcatgttgttagacagtgctttatcaattgctgcacgcaaacgcgggttagcgtccggatcaccaccgcccagcttagccgcggttaccagctcacgaatgattttagtgaagattttaccgcgcttagcatcctgcgcagctttacgatgtctggtgttggcccatttactatgacctgccataaaaatatctccagatagccctgcctgttcaggcagcgttaattacaaactgttcaatcgcctgccggttgctccaggacttagtgagcgccgccgcagcagacgcatcaagccacttgtaagccagatgttcagtgaaaacgatctggcgctcgtgcggaagcgcaagacagaaccatgattccgtattacgcgtcacgcccggcgcatagcgatgacgtaaatgtgaaaaaatttcaaactctaccgtgcgctgacagtcaattaaggtcagttgttcagcgacaacatcaatggtgacctcttcctttacttcgcgcatggcagcttgcggcgcggtttcaccctcttccacgctgccggttaccgactgccagaaatcgggatcgtcacgccgctgcaacatcagcacccgtttcgtatcttgtgcgtagatgaccactaagatcgaaacgggacgcttataagccatatcagttattctcagccttcttcacaacctgaatgctcagctcagccagtgcagtcgggttagcaaagctcggcgcttcagtcatcaaacacgctgccgccgtggttttcgggaaggcgataacgtcacggatattgtcggtgccggtcagcagcatcgtcagacggtcaagaccgaatgccaaacctgcgtgcggcggagtaccgtatttcagggcgtcgagcaggaagccgaatttctcgcgctgttcctcttcgttgatacccagaataccaaacaccgtctgctgcatatcaccattatggatacgcacagaaccaccgcccacttcgtaaccattgatgaccatatcgtaagcgttagccaccgcattttccggtgcagctttcagttctgctgccgtcatgtctttcggtgaggtgaacggatggtgcattgctgtcaggccgccttcaccgtcgtcttcaaacatcgggaagtcgataacccacagcggtgcccatttgctttcgtcggtcagaccaaggtctttacccactttcaggcgcagtgcgcccatcgcgtcggcaacaattttcttgttgtcggcaccgaagaaaatcatatcgccatcttgcgcgccagtacgctccaggatggcttcgatgatttctgcattaaggaacttcgctaccgggctattgataccttccagacctttcgcgcgttcgttaactttgatgtaagccagacctttcgcgccgtagattttaacgaagttaccgtattcgtcgatctgcttacgggtcaacgatgcgccgcccggaacacgcagagcggcaacacggcctttcggatcgttcgccggacctgcaaatactgcaaactcaacagatttcagcagatcggcaacgtcggtcagttccatcgggttacgcagatccggtttatcagaaccataacggcgttctgcttctgcaaaggtcattaccgggaaatcgcccagatccacgcccttcacttccagccacagatgacgcaccagcgcttccatcacttcacgcacttgcggcgcggtcatgaaagaagtttccacatcgatctgagtaaattcaggctgacggtcagcacgcaggtcttcgtcacggaagcatttaacgatctgatagtagcggtcaaagccggacatcatcagtagctgtttgaacaactgcggggattgcggcagcgcgtagaatttacctttgtgcacacgagaaggcaccaggtagtcacgcgcgccttcaggcgtggctttggtcagcatcggagtttcgatgtcgaggaagccgtggtcatccataaaacggcgcaccaggctggtgattttagcgcgggttttcaggcgctgagccatttccgggcgacgcaggtcgaggtagcggtatttcagacgcgcttcttcggtgttgacgtggttagagtcaagcggcagaacatctgcacggttgatgatagtcagcgaggacgccagtacttcgatttcgccagtcgccatatcgcggttaatatttttttcgtcacgcgcacgtacggtgcccgtgacctgaatgcagaactcattacgcagttcagaggccagctttaacgcgtccgcacgatccggatcgaaaaatacctgcacgataccttcgcggtcgcgcatatcgatgaagatcaggctaccaagatcacgacgacggttgacccaaccacacagagtcacctgctgccccacgtgggacaaacggagctgtccacaatattctgtacgcatgagatatcccttaacttagctgccggcggatgccccctgctgcgcaggtgaccaagtcgcagcgttagctgtatgtcacaactgaatgaaaaaaggcggctattatactggaaattctgccgcaccgtaagagcctggcccgcgctggaacgcctcgttaccactttatatcgggcctgaaatcagactctacgccagtttgctataaaggtgttgcccgaactcataaaaattaacaaaatttgtcgttccgccatcggctaatcgcattaaggtgagaggcacgattttgttttgtcaggagtcatcatgcttgaacttaatgctaaaaccaccgcgctggtggtgattgatttacaagaaggcatcttgccttttgccggaggtccacatactgccgatgaggtggttaatcgcgccgggaagctggcggcgaaatttcgcgccagcggtcagcccgtgtttctggtgcgcgttggctggtctgccgattacgccgaagcattaaaacagccggttgatgccccctcccccgcaaaagtgttgcccgaaaactggtggcaacatcctgctgcattaggtgcaaccgacagcgatatcgaaatcatcaaacgtcaatggggtgcgttttacggtacggatctggagttgcaattacgccgccggggtatcgatacaatagtgttatgtgggatctcgaccaatatcggtgttgaatccaccgcccgcaatgcctgggaactcggttttaatctggtgattgccgaagatgcctgtagcgccgctagcgccgagcagcacaataacagcattaatcatatctacccgcgcatcgcccgtgtgcgtagcgttgaagagatcctcaacgcgttatgatttacatcggtttgccacaatggtcgcatcctaaatgggtgcggttggggatcaccagccttgaagagtatgcccgccactttaactgcgtgacgcgggcattttaaaaatcactaaagaacgcccaagagcatgtgttttctttagtttattcaatgcattaaaaaatagtttcgcatgaaattcggtaaacttcatgtgtgcaataatgtcccattcatgccccaaaatgccccaaagcagacatttttgccccaagtatgccccacaagtcacgtcttcaagtcgtctatatccatagcacaccgagttacattcttgcatccggggtgtcgacaatacctactttattgagtgtgcgagaattaccaggaacctttccacaatgtagtagtctaatagtcgaatccatctaacattaagaagcgttatgatcactagcctctcattgatatcttctgtaatagtcactctatgtatcatggtgttcgctacagtaaaggtagggattggtttgtctaacaatccagacagaaatgataattaacctcaaccacgtaaccacacttcatacttcatacttcacttaacagtgaagtgctcacatcaccgggcagtcatcaaactccgcattcctggcatcattaatgatgtacgtgatcactccaaatatagcgggtgcagaactgtaaccatcatcatctgctggcagcgcttcccttctcccgttatccagattaaccaggtgcggctgaggatgagtccgatatcgcttgatcctgaattccccgtcgattgcacatatcagcagtgaaccatcgcaggcagtaagtgacgcatccacaacaagcaacgctccctggattatcccttccctgaaatgtgaacgcgatgcccgcatgaaataagtcgctgcgggctgactgattagctgctgatcgagggagattcgtgtttcaacataatctgccgcaggtgaaggaaatcccatgtttacgccctctcttgaataccggataaaaacacagtataaatactgtatatccatccagcaaagaggcaatgagcaatgttcgtggaactcgtttatgacaaaaggaattttgatggtctgcccggtgcaaaagatatcattctgggcgagttaactaagagagttcaccggatcttccccgatgctgatgttcgggttaaaccgatgatgacactgccggcgatcaacactgacgccagcaagcatgagaaggaacagataagccgtactgttcaggaaatgtttgaagaggctgaattctggttagtgagtgagtaaagattttcaatgcccgccacagttacgtattgattatgctgtggaggatattcattttcgtaaacgttggtttgggagaagcggcaaaacggaatgtgggaacaggggaaaatcagataccagatatgtctgcatttccatctggcaataactggtttcagttaccaagtggacatatcgttcagatattttccatgaacgttcttggtgcagatgctaatggcacgtcagctaattaccccattgcttttccaacaacgatgattgctgtcagtgctctatggtctgatgggactgtagcaaatgcaccgacatacaagatgatggggaacacgactaacagaacaactttgacgataaaagtatcagccagctcaggtacttacgggacaatgattattgcggtgggacgataatatgaataaatacagttactctccttcagaaaatgccttttatgctgttgcgttaaaaaatacctatgaattgagtggcacatggccagctgatgcattagatattcctgatgacatttctgtaaaatatatggcggaaccgccacaagggaaaatccgagttgcaggggaaaatggttttcccacatgggctgaaatacctccaccatcacatgaggaacttattgaacaggccgaatcagagaggcaattattgattaaccaggccaacgaatacatgaacagtaaacaatggcccggtaaagccgctattggtcgtctgaaaggcgaggaactggcacaatataattcgtggctggattatctggacgcactggaactggtcgatacttccggtacgcccgatattgaatggcctacgcctccggcagttcaggccagatgacatccggcgcggtgctggtatctgttgcagtcaccgcgtcaatgtaatccagcacggcgttaagtcgggttgtttctgcctgagtcagtttccgtccggcctgtaatttcagctgaatcagactaatggaagccattgctgcatcaatcagtgattggcgctgtgcttctgccgcttctactgaggcaccgtgttgtgcctcagtatctgtcacccatttctcaccatcccatttatcatatggcgttaacggtgaaagcgtgacataaccgtttttgatggcaccgatataatccactgtaacagctgcgccattttcgattgagtaaacagtctcattgcgatggtcttcctcatggctccatcccttacctgtaaatactgccactcttcccggaatgttttcgtccgggtcaataccagtggaacaggcgggcatacttacgccagtattaatatattcatcagaccagcccgtatattcagacgttactgcatcataataaaaacaacgcatatcacccggcactgcagccagcccattttcatcaaaaacaggtttcattatttagccctcaccagaaagttaaatgcaatatttcgcggtctgacagcaacaaaattcacaccatcacccacagagttactgttgaaattaaatcgtgaaaatcctggctgatttccggcgatgccatcatgaaagttaattgcgtgtccagcacctccgcctatattcccggcaaactgagaaaagtttgtagcttcctgccagcttaataattcgcgaccaccatctgcacctcgcccgtcatcccagacacgaatgaaatcaccgcgggcttcaggtaataccagcgaaggaaacactttcgccagcacaggataatcagtggcagagaatttcgcgccgttgaacttcaaaaacaccatactggaccagctgtcgattacagtatttggcattgcagcggacggccagaagaacggaacgccaatagctggagaaccttctcccaaaccaaggtttgtgcgagcgtctgcggcattcgttgcgccggttccgccgtctgcgacagtaaccgcaccgttgctccctttctgcgcaagtttaccgatgcctgggatggttacggcggtgccgttgatggtaactgtgatgctttggtttgctgaggtggtggcgaacgtctcccacgcgccaatattctcgtcgtactctttgatgagctgtgacatggcctgcgccaggccgtcgactgagatattgtccgacacaaggattccatacttctggccgctcagcgccggggaaactgctggcgtaaccgtcattgacgtggcgctgttcacggatgaaatctgaaacagctgcaccgggttagacatcacgataatcgtctggccagcgcggacctggctggcgggagctgtccagtttgtgccggagccggttgcggtatttccgttaatagagatagttccggtgctataaatcataacaactcctaaatttagacaacatgaagcccggagaggtatataaccctcaccagaaataatttctgaattggtttttaatacatgttgggcaacgccagtgttggcatagctatagttgtatcaaatgccattgaccacccacccaaataataattgccgactactttattcctttctgctctgacattcccacctgtcattacaattcctttatacctaatatttccgtaaccaccaacgtgtcgacagttagccccggtataaacaatctggcaaaattcatcaccaatattcaggttcgcattggcgatatttattgtcccatcaaaaatgaatggcttcgtgcaagtaagaagacgtttgcgttgacgattggtttattcaatccatcaactaaaatatcatgtaaacgaattgccataacatttcccactttattttacttactcaacacaacaagtatattaaaattgaaccttgtcaacaacacaaaggagtcccaatgaaactcgctctaattatgctgccattatgtctgtccctcactgcatgtggtaatggtttaaataccggtaaaccaaattccggtgtcattccaaaacctttggatcgagatggtaacggttctttaatttatgataccgaaaaccttccaatgacggggcagtggtgtcacgagattgatcacgaataccgacgaatcggtagcccttctaactgtgttatagactactaaatattaacccctcaaaagaggggttaatattttaacctgtgaatgaaccagatccgtgtgatactatgatagtaggcgaagaaattgacgcacccctgttattttgagcggacactttaatacgcactgttacgtttggacctgttacaaccgcagaatgcatgataagtctctccatgtcttgcacggatgaaccactctcattgccgttaatgtcgattgttcctgcaccattacctttaacgtttgctataacacagacgtgtcttgcgtgcccagaactggatgaatcattataagtcattaccttttctaaatatccgtcactagaccgactcacattcgtccctgtgtgcatatttgcaatgtccccgataaaactttgggcttccacagtccctttgaatttaccgcttgttgcttggatctcaccagtaaagctaccgccactagcatatactacacctctgacggtcacattgttgaattcagcatctccagctttattcaacttccaaccagcagaaccagctgcatagttgttggactggatatagttaccgatttttgcgttctcaatggtgccgtcctggatgaagctggcccggatgaatgtctgcccgttctggatcacgaacggcaaagctacgctatttccggctgccgtggtgacggcgaagcggtcagccaggaagataacctgcgactgcatgccggatggcgtattctccacgccgatacccatccccgcggcgtaatactgcccgttgctggagacaccaaccttgatgttgtacatcgcgctgagttcgccattaacgttggctatagcctgagcgttagtggtgatggcggaggtatgcccgttcacggtcgccgtgatgccgtttatctgcgtggcggtggcctgctgatagtcggagagcgtctgattcaggctgttgatggatgccttgttgccgttgacgtccgtctgcaggctcagcaatgaacgtgctgtggcttccttctcactgacgatcacctcgtcgagacggtccagattcgcgctgttgccggcgaccgatgcagaaagggttttacgcgtggccacctgagcgaggttggcctggattatcgcaattgcagagttcttcaccccgcccgtcatgccgtccatagaaacgctgatgttgtcgattcgctggcccagggcggtatcagccgtcgcaacggtctgctcaagctgactgagtgaagacgaaacattcccgaccgtgctggaaagctcattaacgctggtctgaaccttcccgacgtcctgggcatttttggcgatatctttcgcttgctgctccagttcgtcgttggcctgtttgatatcgttagccatgccagcaattttttcgttgctgtccaccgcgttctcgatcaggtctttgaacgtttccgactctttcatatcctccagaatgtcattagttatttcgctgacatctatcgaggacgtgcccatgatccagtcggtccagtccccggcgttaccgatacggtcaatcaggcgcgcgcggtaccactggcgaacgccggcaggcatggggccatgctgataatctgcagccgggtacggcaccaggaccagcagttcaggattggcgtagtcggcagttgtggcgcgctgaatctctgtataggccgtgtcgcctgagccatccggaaatttccaggtcaggtcgatatgccagaccacatcttcggtcgccaggaagttgagcggagtacccggttttcccgttttaccggagagataagttgtttcaccgtatccccatggtgacgacgtatcctgcgcattcagcgcccgtacgcgcacgtcatagctgcccgaataaatgccctgaaccgagaaaccctgcgcgctggtaaccggaacgtttatccagtccccgttgtccttacgccactgggcaacataccggattgcgccctctaccttatcccatgacacgtccaggcttgctacagtcagcccctgagacacatgatcgctctcagtcaccacgatattcttcggagcagacaggacgcttatcggcgtgacggtgatcgggggagactcgacccgaacgccgtcatcgatgtaacgatatttgtttggatcgtgctgaacggccgtaatagtgaaaccgcctgtactgtcgtcgttagccgcgattgaggtgaccctgaagtactgtattgcgaggttatcactgtctatcgcccaaacagcgcccgccacaggaacctgactgaatgccgtagccaccgtcaccgtttttttatcggcgctcaccgcgctgattgtccgcgtctgggcttttccgtcgggaaggttaaccaccagccggtctttcgccgcgtagtctatttctcgatcgagggtaatttggcggccgttggccgcgcttatacggcccccgttctccttaccagagcggaaaggatcggcgacaccgataatttcagcgggcaaagggatataaccgtccagccccacgccaaacgatacggtcccgtctttggcattggagagcaatacccagcgaccgcgtcggtgcgcttcactttgcgaggtgcagccgattgcggtcagggacgtctgccggacgtcgtaacgttctacaagcgccgaatcgtaaaccccctcaacggtatcgctgtaatggttctgcggatcggaccaggacaccaggcaggagctgtagcgattcttgtatgagccgcccgcataagtaaacagcccatcgataacgtttgagacgttataaacccagtcaacatcgtcctgcgggacgtctgcctggacataaatctgatcgtttccccagaacgttattccacgaaataccgcggcgagatcgttaagtacctgccaggcgtcctcctggctctgaatgaaaacgttgcaggtgaaacgcggttcggtgccaccggccccgtcggaaaccatttcgtcacagtactgggcgattgaatacagcgcccacttatccaccatggacgcatccacgcgcgtgcccatgccgtaaatttcatccagaaccagatcgtaaaagatccaggcagggttattggaccatgccattttgaacccgccggaccatgaaccagaataggttcgggttttcggatcgtaattatccggaaccttaatcagcttgccttttatcttacaggtcactttcggcgcgctgccgttgaattggctgctgtccacttcgacatacaggagcgctgttaaaggataacgaagcttgctgtcgatgacttccgcatacgaaaacaccttgaaggcgttaaccagtttcgaatttgatccgctggcatcagccgtaatacgcctgaccctgacagaccagccggacgtggattttggcagatcgatacggtggtcacgctgatattccgtcgtggtctttccgtcaaacttgccgtttacaaccgttttccaggcgccgccgtccgttgataaatcgatcgcatactcggtgaccgtgcccaccatatcgccattatctttatagagatactggaccggaaggctgagcttgatacggatggcatccagggaaaggtttgtaaactggcgcgtccagggcgcggtggtggtgacagttgtgcccacggccagctcgttgtcgacctggggcatcccggcaatataggtctggtcctgtgtgcccttgcggaactcccatttcacgccgctgaagttgtattccccgctgtcgtttgccagcggcgtatcgttgagaaaaatgttctgagcggtcaggtcgccctgtatttccccctcagaaacggcaatgagcatttttaattttgcgaccgacagcagatcgtcaggctgctcaaccggagtatgtgaactgccacctccccctttggcaccctgcaggatggtttcttgtttaagaagctgcattttttcacccataaaaaaaggtgccgaagcacctttaagttagtggccgctggcctactgctgatcgctcgagtacataccggcgctgactatcgctccccctgcctcagtcagaccgtaggccagggggacaggatgccccatagcgacggtattgaccggcgccccgaaggcgtagttaggcgtgttgtccgtgctggaggatttacccgcgccgaaggatggctggggcgtgagcatctggacaacgccccccagcatcatcgacactccgacccctgtcagaattgacgtggcgctgatagctgttgcactcatcgccgcgccccaggctgccatgctcgcaccagcggtaaagaatgcagcgaccagcgcaacagccccgacaactatctgcaggacgcccgaacttttggccccctcataaacgggcacgatccggtacacgcttccaccgcgggtcatatcaaactcttccagcccgatattgttgccaccgttaaaaaaggcgaaacggatccccttcatatgagcttcagacatatattttttgaatccgggaacctgtgaacacatggccctgagcatctcgcgcaggtcggcaacatcaaactgaacgcgtttaccgaatttttttgccattttaccttcgagaataagcgtcttaaccatgcattctgtccttatgcctgaccacccggaccgttctgtcgcgataatattttccataaggcgttcgcgaagaaaggtgcccgaaaagatgatggagaatgatgttatcacccacatataccgcggcgtgattagtcaccgatgcctgcacactcatcatgatgatatctccgggctgcattgcaccggcggcaatctcaacgaatccctcacgctcccagttgtcgtcgtagagacgctccttgccgctctcccaccattcgtaaggtactgaataattgccgagaacaatgccgtattcgcgcagataaaattcacggataagcgaccagcagtcggcgtaacccagcacccactgccgcccggcataatcccggtcttcacgcggggaaatcgtacaaaaatccccgtccggccaggacatgatcccccactcaatccccgaccagtcgcactggatccggtccagctctgagggcaccagccgaaccacatccggatgggaatgaatgagcatgatgatctcaccgcgcgcgcgggcagcgagctggtcttccggggagagcgtgaatgtctcctcgggtttatcggcaatgttgcggcagggaataaagatttgttgctggcctgactgaacaatcaggccgcaggcttctttggggtattcagcagcgacgtgctgacggatagcatccagcaatttttcacgcatttttatttcccctgcaggtttgcagccggaaaaccgccgaacggcagcggcgcatccgggccgtgacgatcctgacaatcctgccggcggccgccacaaacatctttcgacgggtcatcggtcggtgtaccgtctttggtaaagtatttcgtgccgttgtaatcgcatccggtcccgcttcggtaccagccccgcatacaccaggtgcagacaggcgtaatctgccgtgtcggcagctgcaggctctgaatatcgaaaggagaacacagctcgaaatcaacctgtacccgcgtctctgcggttttagcattgacgtaaaagagctgtaagcgctcatcggccgggctggcacccggattaccgtttttccagttggcggcatcgagatacttcgaaagcgtggtatggattttgaccttagccctgaccatatcgtcatattcaagacacagcgcggtgacatagtttccgacgttcccgacggacagcgtgggcgttggctgggaacctgtactcgataactccatccccttaagttcgtagggatggggatcgtactggtttccctgccagataatggcgggcagattttctgcggcgaaggctgcccacccctcttcctgaatattgtgcgcatgaaaacgcagcacctgatccataccgaattcagtgccgtcgatctcaatcagctgaataacgctgccgggctcaagctgttgtatgtctgccgtaaaactcatactccccccataaaaaaaagccgcccggaggcagctttcagtgtttttcgagaaaatcagggcgcgaacgcctgttcaaaagtgaaggccacagtggcttttttcccggtagggaaagaaacgctgaacgaatcggccttcattctgaacagctttttttcaccccatggagtggtccaccagaacgatttagtaacgtgagacatcaggaaagcgcgcagcgcagccgcctcctgtctggtgcccgtccagtccaggttccacgtttcctgtttgtcgttgatccccatccccgctatctgtttgtagccatccccgaactgggcctgcagcgttcgggctgtttcagtgccctgcgctgtttttcgcgtgcgccaggtaaacgtgtccgtcactgtgtcctcctcgaataaagcacgccgcccgcggacatttcttttttcagtcgctcggtgattgtctgctgaacaatcgcctgcagctgtttcgccgtccccgtggcgttcgcctgatttatgcttccgtcactcccctgctggctgatgctgactggggcataaacactgatcccgcccatgccagcaccggctgcgttcccgccgccgaccagaccacccgaggcatacccgcgcatcaggcgatagagattagccacgccgatgcggctggttgattctttggtgaagacgaattccccgcggtgaacgataccggctggctcgtacttgccgccgtgcccggtaaaaccgcccacgtcaaaaccctgtggccggtatgacgggaccgcgaatgactgaccggcagaggaggttttcgccccgccgctaacccagcccattgcactctggatggtgtaagccaccagcagctggttgataacggacacaatcattttaaggatcgagctggtgaattccctgaagctcgccttcccggttgtcgtcaggctggtaagctggcccgccaacccgctgaacgtagcctgagaaatctgctgaacggagctgaaaacgtttgtcgctgaatcctgatattcggcccaaccctgtttcgcaccggccagccagtttgcacgcagggcatcttcagcttcgaacgtcgccctttgctcttccagaaccttttgctgcgcctgagggttgtacgaatagctttcgctgagacgctgcagcgtagtttgtcgcccggcttcccgggtggataacccctcagactgagcctgcaggcccgccctggcggctttttgctgctgctcaaacttcacggcctgatcggccagctggttgagcttttgctggctggcaaccttatcgcccaggtcggccagctgccgcttgtactcgagcgtttcttctttgtgcgccagcagggatttttcctgcgccgtaagctgacgacgcccagcggcctcctgcagaacggtgaactgattttcagtttgccagagatcctgacgctgtttacttatgacgtcgttcacgctggtatgctgctcaagcgttttaagctgggcctgaagggtgagaagttcggcctgcgccttttcctcggctttgtccccggcgggcgttgagtagcttttgcctttcggtgtttttggatccttccactgcttttcaatcccggcgcgggccgcggcaatgtccttttcagtccacagcgtggcgacaccgtctttcgcatcctggcggtttttctcaataagctgactgagctttttctctgctgaagcccgcttttctgccgccgtcgcgccggactccaccagctggttaaactgctgctggctgcggattgcctgagcctgctggtccgttcgcattttttcccgcgcggctgccagcccttcctgggcgtattgctgatcggcaagatcgtaagcctgctttttcagctccacctgctggcgcgcgtttctcagcctttccgcatccgctttctgcagaacgttgttaccggcataatccgggtcgaccttaagattgctggacagcgcgcggtactctttctctgctgcctgccactcagcaaaagagtcctggcgcttcatcgcggtgtcaggattacgcccgacgcccagcatcgcatcccacgcaccggaggcggcattcttcacccagttccaggctttttcgagggatccgagattatcctcgaccgcaccggcgcgctgaatgaccgcgtcggaatatgcccgcatggccagctcggcagccttctgagaatcccccagcgcctgagcagaagctatctgttcatactgggtggctgtcagaaaatgaagggaatcgttgagcgtcgcgaccgcgttaaccggatcatccttcaggcgtttaaactgatttatggtttcgtcaacggcctgcccggtagcctgctgcagcctggcggcaacattgctgaccatgctgacgtcattaccgctgaacgcgccgctgccaacgacctgcgccagcacgcctgcagcggcatgctgagtgatgccattacctgccagcgagcgcgccagcgcctgcagctgccctgacgttttccccgcgtagttcccggtcaggatcagctgcctgttaaattcctcagactctttgctgccgtcgtaccaggccttacccaacccgaataccgccgcggcaatccctccgaccatgctggcgatcccaagaccgcgcagtgacagaagctggtctatccaccctgcccggttagccagcgtgatcccggagccgcgcagcgcgccgaagttaccgcgcatgacctcgccgatcagtattcccagttcctgccgggcggcggcactttgcagccccagaccgtgcgtggcgactttggcagcttcgagcttgcggatatagacttcagccgcatcgctggcaccgacctgcgccgccttcatgcgcagtagctcggtaccggagagcttttgctctgcaacctgttgcttcagctggctgaggaatcgcgtgcgcgctgcggccgatttttcctccacgatctgcagttctttttgacgggccgtggtgcgggaaataagggcgagataatcctgctgggttatgttgccctgtgccctcgctgcgcgaaagcgcgcctgcacgttcgcaagcgactgtgtttcaccattgagctggcgtacgccgtcgatctggcggaaaaatgatgccgcaagttcatcctgtcgacgggcaagcgcagcggcctgcccgtcattctcacgcatgcgctgattaagctcggtcacgcggcggtgagtttcatcaacggactttgaaacgttctgccagtctttggtaagcccttccgttgcggccgactggcgggatttcatatctgcggcagccgccgcgccagcgtcacccacggttttaaacgcagccgcctgccgctctgaagcgcgctgcattcgcgtctggactttttcagagtcctcagccatcccggttagctggccctttatgcgggcaacctgctcactaaacgtggcgctgtcgacgtcaaggttgatgaccagatcgctaatctgctgggccatatcggatacctcctgttatcccctcagctgcggccatcagcgcatcatcatccggctcgtcatcgctgatgacgataccggaaggagaaagcaggctgaaatgtgcgggggtaagttccgggtcgcggaagaaaagagtggagatggaataaagcagctctgagaaatgcgcatcgagctgagcgtcctgaaaataatgctcccggtagaactggtgccagtcgcccagctcagtggaagtcattccagccagcatggcgcgccagtcgggtcgcccgaactcgcgcgccagattcaggacaaacttcagctcgctggcaagggcttttccgccgcaacgggttctgcgctttcggcctccgctggggcatccggatcggcagctttgtcatcctcaaccggaacgagcatgccggagagcagctttatttccatttctgctttaccgatcgcctccggcggccagccgctaagcacctgctggtaaagcgtctccacatccgtgccagccggatcgttatgccacaaagacatcgcaatcaaacgcgcaccgcagcgaatatttgagccaatcagcctggccgtcatttcctgatcgctgatgccgtcgctgtcagcgctgacggccttttcctctgcggccataaacgtgatgtactcaatacgctgaagcgccgacagctcgaagatggtcagtgattctttttgccaggtgaacttctcttttttcagaaacatgcgtccttccttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcataagtacctcttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgaatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtgatccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcatgctcattcctcagttgtaaaaaagccccgcgagtgcgaggcgatttgattgaattctcggctcttatctcagcgcagccccttactgcgtgccggttgctcggtgatgagcatcagcgatgagacattaaagccgaccgaaggccagcggcgttcctcatgttgccgacagagccatatcgacaagaggacgaaaactagcagcatgaatcgcctattggttattcgacagtcgcactgattcgtaaatccgctcacacgtcattcctgcccggtagctttcgtcagatcgtccagcataatatcgagctgcttctgcaaggcttccgagcatgtcggcaagcattgctgcgttggctccggctgttttgcttctgacggaagtggcgagatctgcggtgtgctttgcggcgtccatgtgggtagcgagttttgttgcttcggcgcgcagctgcttaacagtggtagccaggccagcagaagtaacggcagcgctcgctgcttgagcttgagcatctttaacggcctcatcccgggcgattgttcgcccttgttcaatcatacgagctgcggtctgtgcattcgcttcctgtgaagattccgcgctatcccggtcagcccactttattttccagctgcggttcgtccactcactgccagcgagaaacgaacctaccaacgcaacaatcacaatgatgcagatgccacccggcttcactggtctatcccccagcacgtcagtgcgctttcctggtcccgccgttctacctgcccataacatccatccttctggcccttggtcaggcggcaatcgcggccaccgtctttaatccaccagcggatagcttcacaggctcctttcgtatcgccagcattaattcgcttatagaacgtagacgggaaacattttccggggccgatgttatatgggcagaaagaagcgatacccgctttctgtggttcggtcagtggtactttgatatttcggtcaacccacgccagcgccttgtcgcgttcaatggcgtttacctgggcgcatttctcagctgacagcttcatgccctgtactactggcttgccatcaaccattgttgcaccacggcaaatggtccagagtccgccgccgtcgcgatatgctgtcaagctgttaccctctttctcatccagaaactgatcgagaatcacgggtgcggaagccccggcaagaatcaaaccaacgaccgctgcgctcaatttattcttcagctttagagacatagccattgcgccgatcctcccgttctttccagcggaaataccagttcactgcacaggtgattaccgtgcatgcgataccgacaataattgcccagtcgctcaggcttaaccctgcaattctgtcggccaacatccaggacacctcttttgctgttttagctgtttcggcatatgccttcgctgatacaccgcagccggcaagcgtggttcctgatccatatgaaagtctgctgtaaatggtgctcattctggtcatagcctcacctccgatagttcggatggcgctgtgtgtgattgaaggggatcaggcaaccgggctcttatgttcaagtaaaaattaaggatgattcccggtgcctgaagatggtgatcaccacagcaacgggggagcgtggtgatcgttatgattttttcagtttttccacctcttcggtggtctgtataaacctgtctgcctccagttctacgccgatcgcccgacggccaagttctattgcagctttcacagttgaaccagagcccataaagaaatcggcaacgatatcccccggtctgctgctggcgctaatgatctgtttcagcatgtcggcaggtttttcgcatggatgtttgcctggataaaactgaacaggcttatgtgtccatacgtcggtataaggaacaagagcggaaacagagaagcagcgccgaaggattttgtattcctccagcaattctgaatacttgcggtttaatgactggtaggtagccaccagctggtggtgaggatgttcaagcttttgctgaatatgtttatcgatggcgatccgcgtgaacagttcctgcaattttcgatagtccacttcattcggtagttgccattggcttgcaccaaaccagtgtgacgccatgtttttctttccggttgcctcagctatttctttcgagctgacacccagtgattcacgggcattacggaagtaatcaatcagcggcgtcataatgtgctgctttagctctgtgcttttcctttcgtaaacatcctctttacctgtatacggtccaagatagtgctcagcaaacaaaatccgttccgtagatggaaagtacgcacgcaggctttctttattacatccattccagcggcccgatggttttgcccaaatgatgtgattcaaaacgttgaatcgggcgcgcatcataatctctatatctgaggccagtcggtgaccgcaaaacaggtagatgctgccagcaggtttaagaacgcgagcatactcagccaggcagctatcaagccagcgtaagtagtcctcgtcccccttccattggttgtcccagccgttgggcttcactttgaagtacggaggatccgtaactataagatcaatagagttatccgggagggtggcgacgtaatgcagactatcagcgttgattaactcaacactgtttatttttacagtatttttcatagatcagtaagcgtaactctgataggctcacgttgcttttgcgctaaagcagtgggccttggttagcttgtgacctgaaagcatgagctgatggctggccgggtgcgctaacacccaccagccgcccatttccacagcagaaaacccccattactggaggcgtttataacatccgaactggtaatcagataaccccgccatcaccagctgcgtaagtatgagctggcaacgttcgtggctgaggtgggtattctgtgcaatctccccagccgttgctggtttatcgcttaattcattgaaaacagcctttgccgtttctgtcatatcttcctgatttagcatgtcttttacctaaaattagttgcgtgacatacagataactctggttggtgataccagcaagagaagaatttgattctgcaaccaacaaggcctttaggcatcaggcaggaatgagatgcaataaaaaaaccacccgaaggtggtcttatatgaatctttaacgcggacttagcaaatattccacatcatcgtactaccgttatggttttcgataatttttgcggctgggctagtaccaaaagagtgcatatagcaatgatgaatagtaaggaccagatcctgcaacgtttggtcactctctagctccatgatatttaaaccaatattttgagctttgtccaaatgaatatgtctggcatgtgcatacgttgcttggtggttgtttaactcatcacatatacgcttagccttagcttcagcgtcagcctgacctgcgaacataccagtacaaagccatttctggacaatttcgttcgcccagagaattgctttttcacactcgccaatcaacgttggatttagtttttggaacgtaaattgccaccattgcagtgcagcagggttggcaaaaatttccgcttttgctctctcatactcctcaataattgcatgagatgataacccattaaactgtggatcaattggccccaagttcgactgtttacctaaaacgatctgctcagcacaacaagcaagcattgtgccacaactcattgaaatcataggtacaatcgctcggatattggttccgaactttgaacgaagataatgaccaattgattctagagctgcgatatcgcctccaggagtatggagtaagatatccaatcccagactcgtatctaacccattgatagcagacataagaccatttttatcatcatctgacatctggatcagatgttgaaacccaggcccccctttttgaaggaagcctgagtaataagaaattacatttcggccagtatgtttcgataaatcacgtaagtacttgtggcgaacctcatccgctggtgtacgttgagcgatagtacccatctcacccaatacgtctatccaatttggcatgttatcaatttatcagtatgagtacagttggtgagattgctgaccgttctgctcagtagtatttggtgttactgtgctgtatgaatagagcacaccacttctcacattcagatcgttttgctgagcgagaacacgcatagcaaaatgctgtacggattcgcctttttgaaactcttggggttgtatgcccattttttcgtaaaattcagcagcgctcatgatatgtccctcgtttttttctacatctatgcaattccaggagccatcaacacaagatgtagtagttagcagtcgtcaaatacacgaaaagcctcaagatgaggcttaaaaagattctttttgataaagatttagccaaactatagcggtcaaaatgcagatttgacaagtataaaaagcacttaaagcctataaataggcagtttttgagaattaaagcatctttaatgaggttgaacaaaatgcagtcttgacgctgaacaggactttactggaacgtagagctaaatggttcgatttcatgaaccagttacaaaaaaacccgctcatcggcgggtttataaaactttggcaacatatcaaatatgcttcaaatatggcttattttgttgcattttgcaagcgtgtttgaaggagatggtgaaatttacttcacatttctgccactttgagggcttcttcttcctcatagtattcaagagccatggccaacgcagattcatcaagctgggtaaaagcggcctttaacccagcccagtgccctgaatagacacgcaaccatgtcgaacggtcaacgctaaccatgcgggccaacgctgcaccagcatagtctttataggtttcattatttctggttgcggcaatttcctgccctgccagccataccaggcctatcagtttctttactacgcgctcctgaagggagttatcacccaggcatttctgataagttttccagacgtattcacacatcatcacctggtgcttatagctaaggtcaaaaccgtagcagtaccgcaaccaggcctgctggtatccactaagcgcggacactgccctacgccacggcgcggactcaaattccgcatcttttatcggcggcattggcctgcggcggctgcgtgtttccagcacatacagtggcgcggaaagtgagttaacaaagcgtggccccttctctccttcgagttcgacgagatgaattccacggcgaggggtggcatttttgtctgctggtgggtgttcactgaaagcctcaagctgcccttttgttcccccagacaggtcaggtagcgcgcggcgcaattctattcttacaaaattcaggtcttgttgattcatgcttctttgcgctccatacacttaagctttcgcaattacgccgatcgccagcgcccgatccataaaacgcagtagcagctcaagctgcgtaccatgcttctgctcgaatgccggtacatcggcgtgtaactcgtcgtggcactctctgcacagagggatcacgaagagatcatgggcttttgttgctgtcccccccataccgtgccctacgatatggtgcggatcatctgctggccgtcggcaacactcacagggttgtgttttaacccagcgggtgtacgtctcatttatccagcggcgtcgctttggcctgagcatgaaagattctggagactccggatcaacagagagcgtgaggatcttcttcgccttctcctgcacgaggctggttgcagaagcggaaggcacaatgtcgctttccctcatgaccgagcggatcttatcatccggaaggcgtagccccttgtgcgcaacgctttccggaataacatcagccaggtcgtttctgaccatccaccagcacagttccggaagcgtcaggatatgcgactcgggaaaaccagaatcacgccgaatgacttccagaatccaggataccaggtttcctgccgctatacctgcaagctgttcggtatgctgccccgacaaagtgtgatcgcaatgccagcacaggcgaatacttcctggtgggtgccgcattgttgtgaagttcttgtcgtgccacgatgaatgtggccactggcattcaaaccgattactcaaccattgctcaagggaaggaagcccaccagcacgctgaataacccgatcattctcgaagacctgccgcattaccggatcatcagccagcggctgaatggcggcgggaacagctcctgtactgaatgacgccatttcttctggttcaggctcaagcagaacgcgaccgcgcataaagaggtgcatcagttccgcgccgggacgaaacaacacaatccccatacgatgggcgatctcgggggtaagcagagctctcacgcgacctgccccctggcaatgtgttctgcccacagtccaccaatccagcgcacgcctttcgccgtgaaacgtgcctggctgaatgcatgatttgaggttacggatgtgccggttttcacttcaaaacggcccgcatcaatatgctgatgccgtggggtcatcgttccgccaagacgatacatgatgtcgttctcaaggaggaataaccgcagatcgggctctttggccttaagcagttttgccacctggcggaatgacattgacccactggctgtacagtaccgatcaacaaacgctaccttcggcgccgcggcagccagttcgttagtcaactgctgtttttgttctgcaaggtcagctgcaagacgtagggcttcagagaatgattgaggaatcgtctgctgctgtgcctgctcaagctcctgccagcgatcaaccagacgcgcggtaaactccggcgacagctgcgcgacaacgatataactgtcccgcttccctatcagataaaccgataccgactgattgaggtgatttttaacttcccccattggggggagttcaataacaccgcgctctgccaggcgttcaatggaccgtttaacatggtcatgtcttgattccaccagctcagcaatatcgctgctggacatggttaacgctgttgttgctaactggctcatacttttctccatatcaggcggctgcacccgccggttcatatctgctgattgttatctctacccgacctttcggcacaacgggtccccattccaccagcatgcgcttaatctggctgtcgtcttcccagacacccgcatgcgtcagcgcgtcaaacagggctttgttgtaattatcgatatcccggcggcgcgcatccggcgggtacagagtgatttctaccgctgccagttcagtcgatggcttcgggagacgtcgtaattgctcaatgatcgccacgcaggcagcgctctggtatttacggccagcggcgctaatgaggtgacgaccggccagcggccccttgttaggggcgcgccagtaagtgttcacgctcggaggaaaaggcaggatcagtttcacgcggcctctccccgcatattgcgaacaagttcagaagcagcagtaatgatttcgctggtggcagtccgctccagccagagttgattgatattggctttcagcttgtgttgcagtgactcatccagcatgtcagcaccatccacctggtcgaatacaattctaacctccagcggccagatacgggactcgggaagcggatccgatactggtttagctttctcacgaatgtgcatgcggatctggcgaatattggaccaactggaaacatccaggcttcccatggctgcaatgaagtcagtgctgttcatgccatattcaccggatgcttcaagggcaacagtgcgaatacgttccgacatatccaggcgcacagcagcgtcatcgaattcaatcgacaacagccactcatccacaccgaacaaaatactctcacgaataagcagcttcgctttgtcgatcgttaaaggtgatacctgagtgaattccggtgcttcgacagaatccgccgcccaggtatgcccaaacttcgattcactgaatgtgtattcttctttatcgccaaacgcagctctaacgcatgcccacgcctcgacaccgctgatagcaaaaatatctttctgggtgagtggcaactctgcttctggcttatcagctgcaggaggtgtggcagttacaggttgagacttgctggcagcaaattgtgccaaagccataaacgcccgcccttttgcctccagttctgtgcggttgatatagctgaaccgctcgccacgccatgacttatcgaatacagctatggcaccggcaaaaaacgcgctggtgggtttctgtttttcgtcagcaggtacaaaccacacaggcagatcgaacccaatgcgcccgcgaatgaatacaatgtgatcggcatcttccggccaccacgtttcactcggcgcggcttttatcaggaatacatagcgaccgcccttctcgcgctgggctgctgcgtagttcatgatgtgcgtcatgccggtgatcgcctgcttctcgtggtactgcgaacggctatacggtgggttgccatagccagcgccacccagttcagccagacgttcagaccagtcctgcgtcagcgcgttatcttcggcggtgtaccatgccgggcatttcgcgttgtcgtcgtcagcaaacaaatccagaactaatggaccaaatagcgcgttgatcccccaaaaaagcagatccggtgtccgccactgatcgccaacctctttcaattcgtgagctggtttgctacgcagtgccgccagcgcctggcaatatttattggtcatcatgaacggaaccccgaattttctggcagtgagtaatcaacactctggaagtttgcgcggctggctgagttagtctcccatttgccgttaacgcgttcaggccggccagcactggaccatttggtcgcgctttgcaggtaaccagggaagttttttggaatgaacagagttgccgggcggaggtattgcgcctgctcgctatcacgccaatcggcatttttgtaatccactaccaagcacaggtcatcaacagtgaattgttcccgaagacgggcgcgaatattctccagcgacgtgctgcatacctggtagcgtgagccagtagtctgattcaggtaagacaaaacctgtctggcctgatcagtaatcacaacctcagggtcgggttgcgccgcaaccggacaagagggttttgaagttacttgtggttcttgttttgattttactgacggatccccaccagattctgacgggtcaaaaccgccttttttgctggatttcgacgcctcaaattttgacgggtcggattttgatgcatcagattttgatgggtcagattttgatgcgtcagattctgacaggtgagaaaaagcagcctctcgcaacttaacgacgttaagctgatatacgttcgatgcgttacggttcccattacggcgctgcttacgggaaagccagccatccttctcaagctgagcaagggccgtccttaccgtactttctccagcaccgatttgacgtgcgatcgtgccaatagacggccagctaacaccctcatcactactgaagtcggccagacgcgccatgatggcaacgctggataacttcatgcctgacgaagcgcatgcatcccatacgtaaccggttaatttagtgctcatggtcgtcctttaattctgtaaatttacgctggaattgttcaagagggctgaagcactcatgatcgtacccttcgcgaaggtatataacgcgctgtgtatctggctcccagcggacaactctgacgggaactccgtagtgatctctgaaccgccggttaacttcagccattcctcgcgccccttctcgttcatctgaacaaatgcttctaccatcaagtctgctggctggtagttgcctccatcagccgcgttatttatgatttccacatagccgaactgggcatctttacccaccagcggcaaacatctgaattgcttagctggtctgaatcggtttacactgttcatgcgttagtttctccactgatacgacacgccaaggcgcccggagctgcacactcgcgggcgtcaccttttctgcctgttgaaacgaatacgtcaatcgcctgatctgaaacaccaaccccataaagcgccataaatcccaggaacccgtgaatctggtggcggagcttcttactgaataattctgaaagcattttgcgctctgatgaatcaattaccccatcagccgctgctgccatcttggcattagccagctcaccagatgctgctgccgctttcatctcaatgctgtacagctcaacgttatccaggctctcagcagttggaacatccaccagccatttcccttttcggttcgcctggtactcggccaagtaacaagaaccagacaggtcctccatccgttccagttctgccaaggtaaagaaccgactgccacacttctggtacaggtggttgtggaactggtcgatagtcatccctaaatcggaagccatacctaagcgaccatgcttgtgtgccttacacatcaggcggattgctgtatttatgctgtctaccatgttgatttccctctggtagttaataatcaacttaaagttgactattgttgttagcggaaggtatgccgtcatttttgttcggataaatatcaggtcgtaattgatggggagttactacccatccgccccattggcagagttgaataactctttcagaaggtactcggttctttgcaatccagttcgcaacagattgaactgattggaattcaaaccgccttgatacctctgaaatcgacccgatcgccttcacagctttagctgttacattcttgtgttgagatgacatgtgttctcctatgactaagcctgcatcaatactacttatagtagcaattattagcaacttaaaatagaaatgacaactatgccttgtgcgcttaatcttctacttatggtggaaaatgctaaatacaaagactttgccgaaaggctaaacaggtctctccaagagcaatctattggagttaaagaattgtcagagttcagtggtgtctcgtatgagatggcgcggcgctacactcttggtactgcaaagccgagagatgagaagatgattcgaattgcagaaagacttgccgtctcaccggcttatcttgattatggtgtgcctgttaatggtggcgacgcgccagccaaaggcacggtcagaatagagcaattggatgttcatgcttcagccggttccggatatataaaccaaccattccctacaatagtgagctcaatagagattccagaagagaggatcttcgagttgtttggtcgtagaagccttgatggcatcgtcatgataaatgttgatggcgatagcatgatgcccacgctttgcccaaaggacctgcttttcatagacagcaaggttgaacaattcagcggcgacggcgtttatgtgttcaattttgaagacagtacgttcgttaaacgtttgcagaaggtaaaagggcgccgactggcagttctttcagacaatgaacattacccgcccttcttcatagaggagcatgaaatgaatgaactatacatattcggcaagctaatcagatgcttacctctaaaaatgatagagtttggctaataattaattcatcaagaaaccggcgaaagccggttttttttacgcctccaattcctcacctcataacactacactactaaaaatttcattttctactttttgttgttgcaattatctacttaaagtagctatagtcattgcatcgaaagcgaacaggcaggacgcccacgaagtagccgccggtggcatatgaataaccggatgattcgctgacagaaaacttaggttgggggtagaggtttacatgaatcatttattcacatgctcattttgcggagcaaccgaactgggagcgataaagatcgtcgcaaaaggtggtaaggacgaacctgccatctgttcggaatgcgtagtcacatgtgtagaaaaaatgatcctgactaaaaaatcagaggctgaaaaaccaacctctgataacgaaataatatcagtcgataaaaaactatttaaagagcttcttcagcttgtcctcaaccttcctgatttcggaagtaagctggctgctgttgacattgatagtagctccacatcgacaagtgaaacttttgttcgacttgagccaagcgattttcttcttcgtcttagtgccgcacttagggcatgcgggtaacgtaatttcctggttatcaaaagcgcccataaacatccctcttggttgtgtgagaacaccaagataccaccgcgcctgatgtggttaaaagcaggctaaagcaataacaagtaactccctgttctggcggcccggtgttttcccgtgtatttccggtaaccgccagcctttttcagggcacaacagaaaagggcatcaccgggcgacgggctcataacccaatccacccgggcaaaaagaaagcggtctctgcaagccgccgaccaatgcaggtgcccttctctgttgtgtatggagaaactaactttttagcgtctgtgcagatgcgctgaggaaccgagaatgaataatccgtttttcaaaaatatgttggtgtatcgcattagtcgcgatttcaccatcaaccaggaagagctggaacagcagcttgaactatttcgcttcactccatgcggtagccaggatatggcaaaaaccggttgggtatcaccacttggtcagctgtcagatcgcttgcatcacactgtcaataatcaagtgttgttggttattcgccgggaagaaaaaatactgccatctcctgtcattactgaagaactgcgcaagcgtgtgtcgcgtctagaatccgatcaggggcgtcgcctcaaaaaaactgagaaagattcgctgcgtgatgaagtgttgcactccctgcttcctcgggcgttctccaaaaactcgactgttggtttgtggatcaacgtcaccgacggtctgatcatggttgatgcagccagcgctaaacgtgccgaagactcactggccctgcttcgtaaaactctcggttctctcccggtggtaccgctgactatggaaacgccgatcgaactaactatgaccgactgggttcgttccggtagtgcgcctgctggctttggcctgggtgatgaagccgaactgaaagctattcttgaagatggcggtattggacgctttaaaaaacagactctggtcagtgacgaaattcatgtgcatctggaagctggcaaagtagttacaaagctgtctatcgactggcaacagcgcattcagttcgttctttgcgatgacggcagcatcaaacgccttaagttctctaatgagattacagaacaaaacgacgatatcgaccgtgaggatgcggctcagcggttcgacgctgactttgttctgatgaccggcgagcttatctctctcattaacggattaacaacctctctcggcggcgaagccaagcgataaacaccaggcaacaattacccccataagcatgggttgggttgctgcacgctaaattcagcaattcattaatttaatggcgcggtgcagcgcgccaatatggagaaaaccatgagctacattcagacattatccggcaaacattttaattacctcgatatccaacaggacgatatcgtgatcgaggatattgctaccgcgttgtctcatatctgccgctttgcagggcatcttcctgagttttacagtgtcggccagcatagcgttttaaccagccacctcgttccgcaggagtttgcattagaagcactgcttcatgatgctgctgaagcctacctgcaggacatcccctccccacttaagcgcctgcttccggattaccaggcaatcgaagctcgtgtggacgcagccattcggcagaagttcggtctaccaactgagcaacacccaaccgtgaaatatgccgacctggtgatgctcgccagcgaacgccgcgattttgagattgacgaaggttccatttggccatgcctcgagggagttgtcccaacggatttattcattatcaacccagttcgtcctggccagtcatacggcatgttcatcaatcgctttaacgagttgatggagcagcgccaatgcgccgcatgaaggtaaaagaactcgtagcggaggcgtttgcctccgttgctgaattgccaccaaagcatgcgccgcttatgcgcgaagtcgccaccagactggacgctacgttcgcagcattaaaagagtctctggtgcaactggaacaggaacgtaaagataaaacgccatgaccgtatttgaatatctccaggctcatccgaataccaccagcggtgaaatcgccaaaggtatgaacaaaaagaccccagcggtcgccggagcattatctcagctctatggcaccggtcggatcgtgaagtctggtgttcgcaagggtattccaacataccgcattaacgatatgccgtttggttgcagtaacagcctaaccatgatgtttaaccagctcttgagcagagccagacaaggagcagcccaatgacagcactcaacaaacaggcgctgcgtgaagaattccagttcatgcaggacaactatagcgacccggcagaccacgatcggcaggtgatttacatcgaggcggaggcgctgctggatgagttggaagccaaagactcaacgatagcagcacaacaacatgagatccgtatgttgctgaatgcgcttgaggaaaaaccatgcccgaaatgcaacgacacaggaatgactgatagtggcggcacgcagccatggggcgagccgattgagattgaatgcgactgccgacagcaggatgccaacaccgcagaacttgtagccgctggcattggcgtgaagggggagtgagatggataaattaatcaaacctaccgccaaaggtaaatatgacggttcatgtgattatctttgctcggaagatgcgcgattcatcgttatgcgcggcgattatacggaagcggaaataattcaggcttctgtgtctcaagatgtaatcgactcggatggtgcggctgattttgcaagtagcgcccgctattatcagtgctggtacaaagttagcccaataggtggtcaggatggctattcaggctggcatcatcctcgtgattcgccgtgtcgcggtgcatatttcgcatcagttttgcaatgggattaaggaggactaacccatgacaactaacaaccacccggcgcacggtcctgtatcactcgatcgcctgcaccagatacgcgaacacctgctgcatgatacccaatactcaaacggcgggaacagagcctacattctcgctgatgtattgaaggtgattgatggggctattgcccgcgagctggtacgccgtgagcatgcagcgtggtcacaggctactttcggcgatgtcggtccagttggtccgctgaagcacctttccaaagaagcgctcgaggctgctgctgaaccaggcgaccttagcgaatgggctgacatgcaattcctgttatgggatgcgcaacgtcgtgccggtatcagtgatgagcagattacccaggcaatgataaaaaagctggctataaataaggttcgccaatggcctgagccgaaagacggggaacctcgattgcatatcaaagaacagtcagagcaggagaaaaaataagaatgtttagcctgattcggcgcggtcaaatctacacggacagtagcaactggcccgtaattatccatagctgtagtgatcactcggtccgaattaaacgcaatgatggcgagctgagaacgattagcatcaaacgctttaacgaagattttgaacgagtggagcatgatgagtatcgcaaaatatgtgccgaaatagagcaggaaacaaacctgaaaaacctacgtgcgatgcgtcgcggcaagattactgaatagccaaacaggagaatatttaacgtgaacaacttaatgatcgaccttgagtccatgggcaaaaaaccgaatgcccctattgtctccattggtgccgtattcttcgatccgcaaagcggtgaactgggtcaggagttttacaccgctgttaatcttgaaagcgctatggagcagggagcggtgccggatggtgacactattctgtggtggttaagacaaagctcagaagcacgatcagcaatctgtgttgatgatgcgatgccgatatcatctgccctatctgaactgagccatttcattaatcggcattctgataaccctaaatatttaaaagtttggggcaatggagctactttcgacaacgttatattgcgcggcgcatatgagcgtgccggccaggtttgcccgtggcaattttggaatgatcacgacgtcagaaccatcgtcacattaggcagatctgtaggtttcgatcctaagcgtgatatgccatttgatggggttgcacataacgcactggctgatgcccgccaccaggcgaaatatgtttcagcgatttggcagaaactaatcccaaccaccagcaacagctaaagttttccccgggtgcagccgggataatggagaaataactatgagcaatattttccagttagctcccaacgattgggtttgtgaaagcgttttgatcgcggttactgggctcaaacccggaaccatcctccgtgccagaaaagaatgctggatgattgggagggagtatatccacgtatcgcctgacggaaatcctaaaccttccagtgagtgcatgtataacagaaaggctgtagatgcctgggtcgcttcaatgaaaagcaagcaaccagggtgatttgatgccatgaaaaaggtaagctcgtatcgctcttgggcgtctggaggtaacaccaatggataaagtcacatatccaacaggcgtcgaaaaccacggtggcacattacgcatctggtttaattttaaaggtaagcgtgtcagggaaagtctcggtgtccctgacaccgctaagaacaggaagatagccggggaactgcggacatcagtatgttttgccatccgcacaggaacctttgattatgcaacccagtttcctgactcccctaacctcaaggcttttggtgtaagtaaaaaagacattacagtgaaagaacttgaagaaaaatggctggatctgaaacggatggaaatctgcgcgaacgcatttaatcgctatgaatctgtcgcaaggaatatggtgccgaggatcggaggtaatcgcctggtgtcagcagtaaccaaagaggaattgctgtatctgaggaaatatttgctaactggttatcagaatccgacgaaaaacaaagccccggcaaaagggcgaagcgttgttactgtgaactattacatgacgacaatggccggaatgtttcagtttgctgcggatcacggttacttagaggtgaacccattcgagggaattaagcctctgaaaaaagccagggcagaaccagatcctctgtctcgtgatgaatttattcgcctgatagatgcatgccggcatcagcagacgaaaaacctgtggtcattagcagtgtacacaggaatgcgtcacggggaactggtctccctggcctgggaagatatcgacctgaaggctggaacaattaccgtcagacgtaattatacgaaacttggtgagttcactctaccgaaaaccgaggcaagcacagatcgagtggtgcatcttatccagcccgcaatcagtatcctgaaaaatcaggctgaaatgacaaggctgggcaggcaatatcacattgaagtgcagttacgtgagtacggccgttcggtgaaccatgagtgtacattcgtctttaatccgcatgtggtcagacgcagtaagcaggtcggatttatctaccgggtcgattcagtaggcgactcatgggaagcggcacttaagcgtgcggggatcagacacagaaaggcgtaccagtcacgacacacctatgcgtgctggtcattatcagctggtgcaaaccctagttttattgccagtcagatggggcatgcgagcgcgcagatggtgttcaatgtttacggtgcatggatggctgacagcagcgcagagcagatcgcaatgctgaatcagaagctggcagattttgccccattgatgccccatagccacgagaacagtacgggaggattattaaaatcagtaagttaacccctaacgcccgtcatgttaactgtgtggagggtaacaccacgctttatgccctgccgaaacccgaggttgtcctgcgctggcgtgagcagaccacagatgacttccgcttctgttttaagtttccggcgaccatttcgcatcaggcagcattacggcattgcgatgatttagtgactgaatttttgacccgcatgtcaccgttggctccgcgcattggacaatactggctgcaactgcctgccacattcggcccacgggagctgcctgcgctttggcattttctcgattctcttcccggtgaatttaattatggggtggaagtccgccatccacagtttttcgccaaaggggaagaggaacaaacgcttaatcgcggtttacatcagcgcggcgttaatcgggtgattttagacagccgcccggttcatgcagcacgtccatacagtgaagctattcgcgacgctcaacgaaaaaaacctaaagttccggtacatgctgtactgacggcgaaaaatccactgatccgttttatcggtagtgatgatatgacgcaaaaccgggaattatttcaggtctggttacaaaaattagcgcagtggcatcagaccactacgccttatctttttttacatacgccagatattgcccaggccccggaactggtacataccctgtgggaagacttacgtaaaacgcttccagagatcggagcagttccggctattccacagcaatcttctcttttctgaatttgccacctatcatagacaggtgccatcggccattttaaagggagtttgtatggtaagcgcgctgtatgccgttttaagtgcgttgttattaatgaagttctcttttgatgtcgttcgcctgcgaatgcagtaccgcgttgcctatggcgacggcggttttagcgaactgcaaagcgctattcgcattcatggtaacgcggtggaatatattcctatcgcgattgtgttgatgctgtttatggaaatgaatggcgcagaaacctggatggtgcatatttgcggcatcgttttgcttgctggtcgtctgatgcattattacggttttcatcaccgtctgttccgctggcgacgttctggcatgagcgccacctggtgtgcgctgttgctgatggtgctggcgaatctttggtatatgccctgggagttggttttctccctgcgttagcgcacaatacgccactttctttttcccggatttttacgttatgtctcaccgcgacacgctattttctgcccctatcgccagactgggcgactggacctttgatgaacgggtagctgaagtcttcccggatatgatccagcgttccgttcccggctattccaatattatttccatgattggtatgttagccgagcgcttcgttcaacctggtacgcaggtttacgatctgggttgttctctgggcgcggcgacgctctcggtgcgtcgcaacattcatcatgataattgcaaaattattgccatcgacaactccccggcgatgattgaacgctgccgtcgtcatattgacgcctataaagcccctacgccagtagacgttattgaaggtgatattcgcgatatcgccattgaaaacgcatcgatggtggtgctgaattttaccctgcaattcctggaaccttccgagcgccaggcgttactggataaaatttatcaagggctgaacccgggcggtgcgctggtgctttcggaaaaattcagtttcgaagatgccaaagttggtgaactgctgttcaacatgcaccacgactttaaacgtgccaacggttacagcgaactggagatcagccagaaacgcagcatgctggaaaacgtgatgctgaccgattccgtggaaacccataaagcacgcctgcataaagccggttttgagcatagcgagctgtggttccagtgctttaactttggttcactggtggcattaaaagcagaggacgctgcatgatcgactttggtaacttttattctctgattgccaaaaatcatctttcacactggctcgaaacgctgcccgcgcagattgctaactggcagcgcgagcagcagcacgggctgtttaagcagtggtccaacgcggtggaatttctgcctgaaattaaaccgtatcgtctggatttattgcatagcgtaaccgccgaaagcgaagagccactgagcgccgggcaaattaagcgcattgaaacgctgatgcgcaacctgatgccgtggcgcaaagggccgttctcactgtatggcgtcaacatcgataccgaatggcgttccgactggaaatgggatcgcgttatgccccatctttctgatttaaccgggcgcaccattcttgatgtcggctgtggcagcggttatcacatgtggcgcatgattggcgcaggggcgcatctggcggtgggtatcgatcccacgcagctattcctctgccagtttgaagcagtgcgtaaactgctgggtaacgatcagcgcgcacatttgttaccgttaggtattgaacaacttccggcactgaaagcctttgataccgtcttttcgatgggcgtgctttatcatcgtcgttcaccgctggagcatctctggcagttaaaagaccaactggtgaatgaaggcgaactggtgctggaaacgctggttattgatggcgacgaaaacacggtgctggtgccgggcgatcgttacgctcaaatgcgtaatgtctatttcattccttccgcgctggcgctgaaaaactggctgaagaagtgtggttttgttgatattcgcattgcagatgtgagcgttaccaccacagaagagcagcgacgcaccgaatggatggtcaccgagtctctggccgattttctcgacccgcatgatccgggtaaaacggtggaaggttatcctgcgcctaaacgcgcggtgctgattgcgcgcaagccgtaaaggtctggtaatactgccggatgcggcgtgaacgccttatccggcctacaaagtcttgctaattcaatatattgcaggggctatgtaggcctgataagcatagcgcatcaggca

SEQ ID NO: 135 - DNA, 972 bp, Escherichia coli
  source 1..972: mol_type = genomic DNA
ttatttgatgggataaagatctttgcgcttatacggttggatttcgcccggtttgcgagttttcagcaattttaatatccaggtgtattgttctggtcgcggaccaacaaaaatctcgacttcttcattcatccgccgcgcaatcgtatgatcatccgcctctaacagatcatccatcggtgggcgcacctgaatcgtcagacgatgcgtcttgccatcataaatcggaaatagcggtacaacgcgcgcacggcacactttcatcaaacgaccaatcgcgggcaacgtcgctttataggtggcaaagaaatcaacaaattcgctgtgttctgggccatgatcctgatcgggtaaataatatccccagtaaccctgacgtaccgactggatgaatggtttaataccatcatttctcgcatgcagacgaccaccaaagcgacggcgcaccgtgttccagacataatcaaaaaccgggttgccctgattatggaacatcgctgccattttctgcccttgcgaggccatcagcatggcaggaatatcgacggcccaaccgtgcggcaccagaaaaatcactttctcgttattacgtcgtatctcttcgatgatctccagcccttgccagtcaacgcgcggctgaattttctccggcccgcgtattgccaactcagccatcattaccatcgcttgcggcgcggtggcaaacatctcatctacaatcgcttcgcgttcagcttcactacgttctggaaagcagagcgacagattgattaacgcacgacggcgtgagctttttcccagtcgtccggcaaaacgtcccagccgtgccagaatgggatcacggaactttggcggcgttaaagcgatacccgccatcgctgctacgcccagccatgctccccagtagcgcgggtggcgaaaggatttatcaaactcaggaatgtattcgctattattttttttcgtttccat

SEQ ID NO: 136 - DNA, 1323 bp, Escherichia coli
  source 1..1323: mol_type = genomic DNA
ttaatcaaaccgtagctgcggcacaatctctttggcctgtgccaggaattcgcgacgatcggagccggtcagcccttcggtacgcggcagttttgccgtcagcgggtttacggcctgctggtttatccatacttcatagtgcagatgcggcccggttgaacgtccggtattaccggaaagcgcgatacggtcgccacgtttcaccttctgtcccggtttcaccaggatcttgcgcaagtgcatataacgcgtggtgtagctgcgaccatgacgaatagccacataataacctgctgcgccactacgtttggcaaccaccacttcaccgtcacccactgaaagcactggcgtaccttgtggcatggcaaaatcaacacctctgtgtggcgcaacgcgaccggtcaccggattagtacgacgcgggttaaagttagatgagatacggaactgtttcgccgtcgggaatcgcaagaatcctttcgccagaccagtaccgttacgatcgtagaatttgccatcttcagcgcggattgcgtaataatctttaccttctgaacgcaaacgtacgcccagcagctggctttgctcacgtttaccatcaagcatttctcgtgacattaacaccgcaaattcatcgccttttttcagtttgcggaaatccatttgccactgcatggctttaatcactgcgctcacttcggcgctggttaaaccggcgtttctggcgctggcaacaaagcttcccccgacggtacctttcagcagattgttgacccactctccttgctgcatttcgctggtcattttaaaaccgttagcggcagtacggtcataggttcgggtttcacgacgagacacttcccaggtgaggcgctgcagttcgccgtccgcggttaatgtccaggagagttgttgaccgattttcaggttacgcaattctttgtcggcagcagccagttgggtgatatcacccatatcaataccatactgattgagaatgctgcttagcgtatcgccagtggaaacaacatattcatgcacgcccgcttcaccggcgattttgtcatccagttcgtcctggggaatggcttcatcttcttgtgcagcttgatcaatcggctcactggcttcaggtaagagcgaacgaatttcgttctgttccagctcaatggttttgacaattggcgtggcatcgcggtgataaacatagggccgccagacagcgacggccagagtaagaacggtgagcgaccccaacataacgcggtgtggtcgcggtaaattattaaacgccagggcgacagagcgggctatctgttgcac

SEQ ID NO: 137 - DNA, 933 bp, Escherichia coli
  source 1..933: mol_type = genomic DNA
ttaatctcctttcaggcagctcgcatactggttggctaattgattcaggaattctgaatagcttgttttacccagtttgatattcgtccccaggggatccaacgttcccatacgaacggatgtccctcgtgcgacgctctcaacgaccgctggcctgaactgtggctcagcaaaaacgcaggttgctttttgctcaaccaactgtgttcttatttcatgtaaacgctgcgcgccaggttgaatctcagggttaacggtaaaatgaccaagcggtgtcagtccgaactgtttttcgaaatagccgtaagcatcgtgaaaaacgaaataacctttccccttgagcggcgcgagctcgttaccaacctgcttttcggttgaggctaattgtgcctcaaaatccttcaggttggcgtcaagtttggctcgactttgcggcataagttccactaattttccatggattgcaaccgctgtagcccgcgctatctctggggaaagccaaagatgcatgttgaaatcgccgtgatggtgatcttcgtcacttttttccgcgtggtcgtgatcatcatcatcgccgtgaatacttttcatcagcagcggtttcacattctctagctgcgcaatcgttacctgtttcgcttcaggtaatttacttaccggtttttgcatgaacgcttccatctccgggccaacccaaacgactaagtccgcgttctgtaagcgttttacatctgatggacgcagtgaataatcatgttctgaagccccgtcaggtagtaaaacctccgtttctgttaccccatcagcaatggcagaagcgatgaacccaacgggtttaagcgaagcgacaacggcagcatctgcggcctgtgttgcaccgccccagagagcggcggataatgctgcgaaaagaagcgtttttttatgtaacat

SEQ ID NO: 138 - DNA, 756 bp, Escherichia coli
  source 1..756: mol_type = genomic DNA
atgacaagtctggtttccctggaaaatgtctcggtttcttttggccaacgccgcgtcctctctgatgtgtcgctggaacttaaacctggaaaaattttgactttacttgggccaaacggcgcaggtaagtcgacactggtacgggtagtgctcgggctggtaacacccgatgaaggggttatcaagcgcaacggaaaactgcgcatcggctatgtaccgcagaagctgtatctcgacaccacgttgccactgaccgtaaaccgttttttacgcttacgccctggcacacataaagaagatattttgcctgcactgaaacgtgtccaggccgggcatctgattaacgcaccgatgcaaaagctctcgggtggcgaaacgcagcgtgtactgttagcgcgagcattgttaaatcgaccgcaattattagtgctggatgaacccactcagggcgtggatgtgaatggtcaggtggcgttatatgaccttattgaccaactgcgtcgcgaactggattgtggcgttttaatggtatctcacgatctgcatctggtaatggcaaaaaccgatgaagtgctttgcctgaatcaccacatttgttgttccggcacaccggaagttgtttccctgcatccggagtttatttctatgtttggtcctcgtggtgctgaacaactgggtatctatcgccatcatcataatcatcgtcacgatttacagggacgaattgttttgcgtcggggaaatgatcgctcatga

SEQ ID NO: 139 - DNA, 786 bp, Escherichia coli
  source 1..786: mol_type = genomic DNA
atgattgaattattatttcccggttggttagccgggatcatgctcgcctgtgccgcgggtccgctgggttcgtttgtagtctggcgtcgtatgtcttatttcggtgatacgctggctcatgcctcattacttggcgtcgcgtttggtttgttgctggacgtgaatccattctatgcggtgattgccgttacgctgctgctggcgggcggtctggtatggctggagaagcgtccacagctggcgatcgacacgttattagggattatggcgcacagtgccctgtcgctgggcctggtggtcgttagtctgatgtctaatattcgtgttgatttgatggcttacctgttcggtgatttactggcagtgacgccagaagatctcatctctattgcgattggcgtggtcatcgtggtggctattttgttctggcaatggcgcaatttgctgtcgatgacgattagcccggatctggcgtttgttgatggtgtgaaattacagcgcgtgaaattgttgttgatgctggtgacggcattgacgattggtgtagcgatgaaattcgtcggcgcgttgattattacttcactgctgattattcctgctgctactgcacgtcgctttgcccgcacgccggaacagatggctggtgtcgctgttttggtggggatggtggcagtgactggcggtttaaccttttccgcattttacgatacacctgcaggcccgtcggtggtgctatgcgcggcactgttatttattatcagtatgatgaaaaagcaggccagctaa

SEQ ID NO: 140 - DNA, 1011 bp, Escherichia coli
  source 1..1011: mol_type = genomic DNA
ttacggcatttctggcggcgtgatgccgaagtggttccacgcccgcactgtcgccatacgcccgcgcggtgtacgctgcaaaaagccttgctgaatcaaataaggttccagtacatcctcaatggtttcacgttcttcgccaatggctgccgccaggttatccagacctaccggcccaccaaagaacttatcgattaccgccagcaacaatttgcggtccatataatcgaaaccttcagcatcgacattcaacatatccagcgcctgagcagcgatatctgccgagatggtgccatcgtgcttcacttcagcgaaatcacgcactcgacgcagcagacggttggcaatacgtggcgtaccgcgcgcacgacgagcaacttccagcgcgccgtcatcactcatctcaagccccataaagcgtgcgctgcgactgacgatatattgcagatccggcacctgataaaactccagacgttgcacaataccaaaacgatcgcgcaacggtgatgtcagcgaacctgcgcgcgtggttgcaccaatcagggtaaacggcggcaaatcaattttaatggagcgtgccgccggaccttcaccaatcatgatatccagttggtaatcttccattgccggatacaacacctcttccaccactggtgaaagacggtggatctcatcaataaacagtacatcgtgtggttcaaggttagtgagcattgctgccagatcgcccgccttttccagcaccggaccagaagtcgtgcgtaaattaacgcccatttcattggcgacaatattggcaagcgtagttttacccaaccccggaggaccaaaaatcaatagatgatcgagggcatcgccgcgcagtttcgctgctttgatgaaaatctccatctgcgaacgaacctgcggctgaccaacatactcttccagtaatttagggcgaatggcgcgatctgccacatcttccggcaaagtggtaccggcagaaatcagacggtctgcttcaatcat

SEQ ID NO: 141 - DNA, 612 bp, Escherichia coli
  source 1..612: mol_type = genomic DNA
tcataacgcggcgcgtagggcttcgcgaattaatgtttcactgctggcgtcagggcgagcgattttgctcaccatgcggcttgcttcttgtggtttatagcccagtgccaccagcgcagcaaccgcttcctgttcagcatcgtcggtcgccgggctggcaggagacgtgagtaccaggtcggcggctggcgtaaagagatcgccatgcaaacctttaaatcggtctttcatttcgacaatcaagcgttcggcggtttttttgccaatacccggcagtttcaccagtgcccccacttcttcacgctcaacggcattaacgaactgctgcgctgacattccggagaggatcgccagcgccaacttcgggccgacgccgttggttttgatcaactctttgaacaacgtgcgctcttgtttattgttaaaaccgtacagcagttgcgcgtcttcacgcaccacaaagtgggtgaaaacgatcgcttcctgacccgcttcagggagttcataaaaacaggtcatcggcatatgcacttcatagcctacgccgcccacttcaattaacaccagcgggggttgtttttcaatgatgatgcctctgagtctgcctatcac

SEQ ID NO: 142 - DNA, 603 bp, Escherichia coli
  source 1..603: mol_type = genomic DNA
gtgaatattaattatcctgctgaatatgaaattggtgatatcgtctttacatgtataagtgctgccttatttggtcaaatatcagctgcatcaaattgctggagtaatcacgtcgggatcattatcggtcataacggtgaagactttctggttgcagaaagccgtgttcccctctcaaccatcactacgctatcccgttttattaaacgctctgctaatcaacgctatgctataaagcgattagacgccggactaacagaacaacaaaatcaacgaattgttgaacaggttccttcccggctacgcaaaatttaccacaccggttttaaatacgaatcttcgcgccagttctgttcaaaatttgtttttgatatttataaagaggcgctatgtattccggtgggtgaaatagagacgtttggagaattgttaaatagcaatccaaatgcaaaactcactttctggaaattctggttcttaggttctattccgtgggagcgtaaaaccgtcacgccagccagtttgtggcatcatccgggtttggtgttgattcacgcggtgggagttgaaacgcctcagcctgaactgaccgaggcggtataa

SEQ ID NO: 143 - DNA, 522 bp, Escherichia coli
  source 1..522: mol_type = genomic DNA
ttaacgcagtcgccctctcgccaggttcagtcgcgattcgctcatttgcatcgcattctgactaacgtggcagtgggtgatggcaatcgccagcgcatcggcggcatccgcctgtggattagcgggcagtttcagcaaggtgcggaccatatgctgcacctggcttttttcggcactaccaatacctaccactgtttgctttacctgacgtgccgcatattcaaataccggcaattcctgattcaccgccgccacaatcgccacgccgcgcgcctgccccagtttcagggctgagtcagcgttcttcgccataaagacctgttcaatggcgaaataatcaggctggaattgggtgatgatttccgtcacgcccgcatagatgagcttcagacgagacggtaaatcatccactttggtgcgtatgcatccgctacccaggtaggacagttgcctgcctacctggcggatgacgccatagccggtcacgcgcgaacccgggtcaatgccgagaataatagccat

SEQ ID NO: 144 - DNA, 741 bp, Escherichia coli
  source 1..741: mol_type = genomic DNA
tcagagagtcgctgcaacctcatcagagatttcaccgttatggtaaacttcctgcacgtcgtcgcaatcttccagcatatcgatcagacgcatcagtttcggtgcggtttctgcatccatatcagctttggtggacgggatcatggaaacttccgcgctgtctgctttcagacctgccgcttccagagcgtcgcgtactttgcccatttcttcccatgcagtgtagacatcaatcgcgccgtcatcataggtcacaacgtcttcagcaccggcttccagggctgcttccatgatggtgtcttcatcgcctttctcgaaggagatcacgccttttttgctgaacaaataagctacggaaccatcagtaccgaggttaccgccacatttgctgaatgcatgacgcacttcagcaacggtacggttgcggttgtcagacagacattcaatcatgattgccgtgccgccaggaccgtaaccttcgtagatgatggtttccatgtttgcatcatcatcaccgcccacacctcgtgcaattgcgcggttcagagtgtcacgggtcatgttgttagacagtgctttatcaattgctgcacgcaaacgcgggttagcgtccggatcaccaccgcccagcttagccgcggttaccagctcacgaatgattttagtgaagattttaccgcgcttagcatcctgcgcagctttacgatgtctggtgttggcccatttactatgacctgccat

SEQ ID NO: 145 - DNA, 444 bp, Escherichia coli
  source 1..444: mol_type = genomic DNA
tcaggcagcgttaattacaaactgttcaatcgcctgccggttgctccaggacttagtgagcgccgccgcagcagacgcatcaagccacttgtaagccagatgttcagtgaaaacgatctggcgctcgtgcggaagcgcaagacagaaccatgattccgtattacgcgtcacgcccggcgcatagcgatgacgtaaatgtgaaaaaatttcaaactctaccgtgcgctgacagtcaattaaggtcagttgttcagcgacaacatcaatggtgacctcttcctttacttcgcgcatggcagcttgcggcgcggtttcaccctcttccacgctgccggttaccgactgccagaaatcgggatcgtcacgccgctgcaacatcagcacccgtttcgtatcttgtgcgtagatgaccactaagatcgaaacgggacgcttataagccat

SEQ ID NO: 146 - DNA, 1773 bp, Escherichia coli
  source 1..1773: mol_type = genomic DNA
tcagttattctcagccttcttcacaacctgaatgctcagctcagccagtgcagtcgggttagcaaagctcggcgcttcagtcatcaaacacgctgccgccgtggttttcgggaaggcgataacgtcacggatattgtcggtgccggtcagcagcatcgtcagacggtcaagaccgaatgccaaacctgcgtgcggcggagtaccgtatttcagggcgtcgagcaggaagccgaatttctcgcgctgttcctcttcgttgatacccagaataccaaacaccgtctgctgcatatcaccattatggatacgcacagaaccaccgcccacttcgtaaccattgatgaccatatcgtaagcgttagccaccgcattttccggtgcagctttcagttctgctgccgtcatgtctttcggtgaggtgaacggatggtgcattgctgtcaggccgccttcaccgtcgtcttcaaacatcgggaagtcgataacccacagcggtgcccatttgctttcgtcggtcagaccaaggtctttacccactttcaggcgcagtgcgcccatcgcgtcggcaacaattttcttgttgtcggcaccgaagaaaatcatatcgccatcttgcgcgccagtacgctccaggatggcttcgatgatttctgcattaaggaacttcgctaccgggctattgataccttccagacctttcgcgcgttcgttaactttgatgtaagccagacctttcgcgccgtagattttaacgaagttaccgtattcgtcgatctgcttacgggtcaacgatgcgccgcccggaacacgcagagcggcaacacggcctttcggatcgttcgccggacctgcaaatactgcaaactcaacagatttcagcagatcggcaacgtcggtcagttccatcgggttacgcagatccggtttatcagaaccataacggcgttctgcttctgcaaaggtcattaccgggaaatcgcccagatccacgcccttcacttccagccacagatgacgcaccagcgcttccatcacttcacgcacttgcggcgcggtcatgaaagaagtttccacatcgatctgagtaaattcaggctgacggtcagcacgcaggtcttcgtcacggaagcatttaacgatctgatagtagcggtcaaagccggacatcatcagtagctgtttgaacaactgcggggattgcggcagcgcgtagaatttacctttgtgcacacgagaaggcaccaggtagtcacgcgcgccttcaggcgtggctttggtcagcatcggagtttcgatgtcgaggaagccgtggtcatccataaaacggcgcaccaggctggtgattttagcgcgggttttcaggcgctgagccatttccgggcgacgcaggtcgaggtagcggtatttcagacgcgcttcttcggtgttgacgtggttagagtcaagcggcagaacatctgcacggttgatgatagtcagcgaggacgccagtacttcgatttcgccagtcgccatatcgcggttaatatttttttcgtcacgcgcacgtacggtgcccgtgacctgaatgcagaactcattacgcagttcagaggccagctttaacgcgtccgcacgatccggatcgaaaaatacctgcacgataccttcgcggtcgcgcatatcgatgaagatcaggctaccaagatcacgacgacggttgacccaaccacacagagtcacctgctgccccacgtgggacaaacggagctgtccacaatattctgtacgcat

SEQ ID NO: 147 - DNA, 567 bp, Escherichia coli
  source 1..567: mol_type = genomic DNA
atgcttgaacttaatgctaaaaccaccgcgctggtggtgattgatttacaagaaggcatcttgccttttgccggaggtccacatactgccgatgaggtggttaatcgcgccgggaagctggcggcgaaatttcgcgccagcggtcagcccgtgtttctggtgcgcgttggctggtctgccgattacgccgaagcattaaaacagccggttgatgccccctcccccgcaaaagtgttgcccgaaaactggtggcaacatcctgctgcattaggtgcaaccgacagcgatatcgaaatcatcaaacgtcaatggggtgcgttttacggtacggatctggagttgcaattacgccgccggggtatcgatacaatagtgttatgtgggatctcgaccaatatcggtgttgaatccaccgcccgcaatgcctgggaactcggttttaatctggtgattgccgaagatgcctgtagcgccgctagcgccgagcagcacaataacagcattaatcatatctacccgcgcatcgcccgtgtgcgtagcgttgaagagatcctcaacgcgttatga

SEQ ID NO: 148 - DNA, 390 bp, Escherichia coli
  source 1..390: mol_type = genomic DNA
tcacatcaccgggcagtcatcaaactccgcattcctggcatcattaatgatgtacgtgatcactccaaatatagcgggtgcagaactgtaaccatcatcatctgctggcagcgcttcccttctcccgttatccagattaaccaggtgcggctgaggatgagtccgatatcgcttgatcctgaattccccgtcgattgcacatatcagcagtgaaccatcgcaggcagtaagtgacgcatccacaacaagcaacgctccctggattatcccttccctgaaatgtgaacgcgatgcccgcatgaaataagtcgctgcgggctgactgattagctgctgatcgagggagattcgtgtttcaacataatctgccgcaggtgaaggaaatcccat

SEQ ID NO: 149 - DNA, 243 bp, Escherichia coli
  source 1..243: mol_type = genomic DNA
atgttcgtggaactcgtttatgacaaaaggaattttgatggtctgcccggtgcaaaagatatcattctgggcgagttaactaagagagttcaccggatcttccccgatgctgatgttcgggttaaaccgatgatgacactgccggcgatcaacactgacgccagcaagcatgagaaggaacagataagccgtactgttcaggaaatgtttgaagaggctgaattctggttagtgagtgagtaa

SEQ ID NO: 150 - DNA, 381 bp, Escherichia coli
  source 1..381: mol_type = genomic DNA
atgctgtggaggatattcattttcgtaaacgttggtttgggagaagcggcaaaacggaatgtgggaacaggggaaaatcagataccagatatgtctgcatttccatctggcaataactggtttcagttaccaagtggacatatcgttcagatattttccatgaacgttcttggtgcagatgctaatggcacgtcagctaattaccccattgcttttccaacaacgatgattgctgtcagtgctctatggtctgatgggactgtagcaaatgcaccgacatacaagatgatggggaacacgactaacagaacaactttgacgataaaagtatcagccagctcaggtacttacgggacaatgattattgcggtgggacgataa

SEQ ID NO: 151 - DNA, 444 bp, Escherichia coli
  source 1..444: mol_type = genomic DNA
atgaataaatacagttactctccttcagaaaatgccttttatgctgttgcgttaaaaaatacctatgaattgagtggcacatggccagctgatgcattagatattcctgatgacatttctgtaaaatatatggcggaaccgccacaagggaaaatccgagttgcaggggaaaatggttttcccacatgggctgaaatacctccaccatcacatgaggaacttattgaacaggccgaatcagagaggcaattattgattaaccaggccaacgaatacatgaacagtaaacaatggcccggtaaagccgctattggtcgtctgaaaggcgaggaactggcacaatataattcgtggctggattatctggacgcactggaactggtcgatacttccggtacgcccgatattgaatggcctacgcctccggcagttcaggccagatga

SEQ ID NO: 152 - DNA, 594 bp, Escherichia coli
  source 1..594: mol_type = genomic DNA
ctacgcctccggcagttcaggccagatgacatccggcgcggtgctggtatctgttgcagtcaccgcgtcaatgtaatccagcacggcgttaagtcgggttgtttctgcctgagtcagtttccgtccggcctgtaatttcagctgaatcagactaatggaagccattgctgcatcaatcagtgattggcgctgtgcttctgccgcttctactgaggcaccgtgttgtgcctcagtatctgtcacccatttctcaccatcccatttatcatatggcgttaacggtgaaagcgtgacataaccgtttttgatggcaccgatataatccactgtaacagctgcgccattttcgattgagtaaacagtctcattgcgatggtcttcctcatggctccatcccttacctgtaaatactgccactcttcccggaatgttttcgtccgggtcaataccagtggaacaggcgggcatacttacgccagtattaatatattcatcagaccagcccgtatattcagacgttactgcatcataataaaaacaacgcatatcacccggcactgcagccagcccattttcatcaaaaacaggtttcat

SEQ ID NO: 153 - DNA, 933 bp, Escherichia coli
  source 1..933: mol_type = genomic DNA
ttatttagccctcaccagaaagttaaatgcaatatttcgcggtctgacagcaacaaaattcacaccatcacccacagagttactgttgaaattaaatcgtgaaaatcctggctgatttccggcgatgccatcatgaaagttaattgcgtgtccagcacctccgcctatattcccggcaaactgagaaaagtttgtagcttcctgccagcttaataattcgcgaccaccatctgcacctcgcccgtcatcccagacacgaatgaaatcaccgcgggcttcaggtaataccagcgaaggaaacactttcgccagcacaggataatcagtggcagagaatttcgcgccgttgaacttcaaaaacaccatactggaccagctgtcgattacagtatttggcattgcagcggacggccagaagaacggaacgccaatagctggagaaccttctcccaaaccaaggtttgtgcgagcgtctgcggcattcgttgcgccggttccgccgtctgcgacagtaaccgcaccgttgctccctttctgcgcaagtttaccgatgcctgggatggttacggcggtgccgttgatggtaactgtgatgctttggtttgctgaggtggtggcgaacgtctcccacgcgccaatattctcgtcgtactctttgatgagctgtgacatggcctgcgccaggccgtcgactgagatattgtccgacacaaggattccatacttctggccgctcagcgccggggaaactgctggcgtaaccgtcattgacgtggcgctgttcacggatgaaatctgaaacagctgcaccgggttagacatcacgataatcgtctggccagcgcggacctggctggcgggagctgtccagtttgtgccggagccggttgcggtatttccgttaatagagatagttccggtgctataaatcat

SEQ ID NO: 154 - DNA, 3927 bp, Escherichia coli
  source 1..3927: mol_type = genomic DNA
ttaacctgtgaatgaaccagatccgtgtgatactatgatagtaggcgaagaaattgacgcacccctgttattttgagcggacactttaatacgcactgttacgtttggacctgttacaaccgcagaatgcatgataagtctctccatgtcttgcacggatgaaccactctcattgccgttaatgtcgattgttcctgcaccattacctttaacgtttgctataacacagacgtgtcttgcgtgcccagaactggatgaatcattataagtcattaccttttctaaatatccgtcactagaccgactcacattcgtccctgtgtgcatatttgcaatgtccccgataaaactttgggcttccacagtccctttgaatttaccgcttgttgcttggatctcaccagtaaagctaccgccactagcatatactacacctctgacggtcacattgttgaattcagcatctccagctttattcaacttccaaccagcagaaccagctgcatagttgttggactggatatagttaccgatttttgcgttctcaatggtgccgtcctggatgaagctggcccggatgaatgtctgcccgttctggatcacgaacggcaaagctacgctatttccggctgccgtggtgacggcgaagcggtcagccaggaagataacctgcgactgcatgccggatggcgtattctccacgccgatacccatccccgcggcgtaatactgcccgttgctggagacaccaaccttgatgttgtacatcgcgctgagttcgccattaacgttggctatagcctgagcgttagtggtgatggcggaggtatgcccgttcacggtcgccgtgatgccgtttatctgcgtggcggtggcctgctgatagtcggagagcgtctgattcaggctgttgatggatgccttgttgccgttgacgtccgtctgcaggctcagcaatgaacgtgctgtggcttccttctcactgacgatcacctcgtcgagacggtccagattcgcgctgttgccggcgaccgatgcagaaagggttttacgcgtggccacctgagcgaggttggcctggattatcgcaattgcagagttcttcaccccgcccgtcatgccgtccatagaaacgctgatgttgtcgattcgctggcccagggcggtatcagccgtcgcaacggtctgctcaagctgactgagtgaagacgaaacattcccgaccgtgctggaaagctcattaacgctggtctgaaccttcccgacgtcctgggcatttttggcgatatctttcgcttgctgctccagttcgtcgttggcctgtttgatatcgttagccatgccagcaattttttcgttgctgtccaccgcgttctcgatcaggtctttgaacgtttccgactctttcatatcctccagaatgtcattagttatttcgctgacatctatcgaggacgtgcccatgatccagtcggtccagtccccggcgttaccgatacggtcaatcaggcgcgcgcggtaccactggcgaacgccggcaggcatggggccatgctgataatctgcagccgggtacggcaccaggaccagcagttcaggattggcgtagtcggcagttgtggcgcgctgaatctctgtataggccgtgtcgcctgagccatccggaaatttccaggtcaggtcgatatgccagaccacatcttcggtcgccaggaagttgagcggagtacccggttttcccgttttaccggagagataagttgtttcaccgtatccccatggtgacgacgtatcctgcgcattcagcgcccgtacgcgcacgtcatagctgcccgaataaatgccctgaaccgagaaaccctgcgcgctggtaaccggaacgtttatccagtccccgttgtccttacgccactgggcaacataccggattgcgccctctaccttatcccatgacacgtccaggcttgctacagtcagcccctgagacacatgatcgctctcagtcaccacgatattcttcggagcagacaggacgcttatcggcgtgacggtgatcgggggagactcgacccgaacgccgtcatcgatgtaacgatatttgtttggatcgtgctgaacggccgtaatagtgaaaccgcctgtactgtcgtcgttagccgcgattgaggtgaccctgaagtactgtattgcgaggttatcactgtctatcgcccaaacagcgcccgccacaggaacctgactgaatgccgtagccaccgtcaccgtttttttatcggcgctcaccgcgctgattgtccgcgtctgggcttttccgtcgggaaggttaaccaccagccggtctttcgccgcgtagtctatttctcgatcgagggtaatttggcggccgttggccgcgcttatacggcccccgttctccttaccagagcggaaaggatcggcgacaccgataatttcagcgggcaaagggatataaccgtccagccccacgccaaacgatacggtcccgtctttggcattggagagcaatacccagcgaccgcgtcggtgcgcttcactttgcgaggtgcagccgattgcggtcagggacgtctgccggacgtcgtaacgttctacaagcgccgaatcgtaaaccccctcaacggtatcgctgtaatggttctgcggatcggaccaggacaccaggcaggagctgtagcgattcttgtatgagccgcccgcataagtaaacagcccatcgataacgtttgagacgttataaacccagtcaacatcgtcctgcgggacgtctgcctggacataaatctgatcgtttccccagaacgttattccacgaaataccgcggcgagatcgttaagtacctgccaggcgtcctcctggctctgaatgaaaacgttgcaggtgaaacgcggttcggtgccaccggccccgtcggaaaccatttcgtcacagtactgggcgattgaatacagcgcccacttatccaccatggacgcatccacgcgcgtgcccatgccgtaaatttcatccagaaccagatcgtaaaagatccaggcagggttattggaccatgccattttgaacccgccggaccatgaaccagaataggttcgggttttcggatcgtaattatccggaaccttaatcagcttgccttttatcttacaggtcactttcggcgcgctgccgttgaattggctgctgtccacttcgacatacaggagcgctgttaaaggataacgaagcttgctgtcgatgacttccgcatacgaaaacaccttgaaggcgttaaccagtttcgaatttgatccgctggcatcagccgtaatacgcctgaccctgacagaccagccggacgtggattttggcagatcgatacggtggtcacgctgatattccgtcgtggtctttccgtcaaacttgccgtttacaaccgttttccaggcgccgccgtccgttgataaatcgatcgcatactcggtgaccgtgcccaccatatcgccattatctttatagagatactggaccggaaggctgagcttgatacggatggcatccagggaaaggtttgtaaactggcgcgtccagggcgcggtggtggtgacagttgtgcccacggccagctcgttgtcgacctggggcatcccggcaatataggtctggtcctgtgtgcccttgcggaactcccatttcacgccgctgaagttgtattccccgctgtcgtttgccagcggcgtatcgttgagaaaaatgttctgagcggtcaggtcgccctgtatttccccctcagaaacggcaatgagcatttttaattttgcgaccgacagcagatcgtcaggctgctcaaccggagtatgtgaactgccacctccccctttggcaccctgcaggatggtttcttgtttaagaagctgcattttttcacccat

SEQ ID NO: 155 - DNA, 618 bp, Escherichia coli
  source 1..618: mol_type = genomic DNA
ctactgctgatcgctcgagtacataccggcgctgactatcgctccccctgcctcagtcagaccgtaggccagggggacaggatgccccatagcgacggtattgaccggcgccccgaaggcgtagttaggcgtgttgtccgtgctggaggatttacccgcgccgaaggatggctggggcgtgagcatctggacaacgccccccagcatcatcgacactccgacccctgtcagaattgacgtggcgctgatagctgttgcactcatcgccgcgccccaggctgccatgctcgcaccagcggtaaagaatgcagcgaccagcgcaacagccccgacaactatctgcaggacgcccgaacttttggccccctcataaacgggcacgatccggtacacgcttccaccgcgggtcatatcaaactcttccagcccgatattgttgccaccgttaaaaaaggcgaaacggatccccttcatatgagcttcagacatatattttttgaatccgggaacctgtgaacacatggccctgagcatctcgcgcaggtcggcaacatcaaactgaacgcgtttaccgaatttttttgccattttaccttcgagaataagcgtcttaaccat

SEQ ID NO: 156 - DNA, 720 bp, Escherichia coli
  source 1..720: mol_type = genomic DNA
ttaaccatgcattctgtccttatgcctgaccacccggaccgttctgtcgcgataatattttccataaggcgttcgcgaagaaaggtgcccgaaaagatgatggagaatgatgttatcacccacatataccgcggcgtgattagtcaccgatgcctgcacactcatcatgatgatatctccgggctgcattgcaccggcggcaatctcaacgaatccctcacgctcccagttgtcgtcgtagagacgctccttgccgctctcccaccattcgtaaggtactgaataattgccgagaacaatgccgtattcgcgcagataaaattcacggataagcgaccagcagtcggcgtaacccagcacccactgccgcccggcataatcccggtcttcacgcggggaaatcgtacaaaaatccccgtccggccaggacatgatcccccactcaatccccgaccagtcgcactggatccggtccagctctgagggcaccagccgaaccacatccggatgggaatgaatgagcatgatgatctcaccgcgcgcgcgggcagcgagctggtcttccggggagagcgtgaatgtctcctcgggtttatcggcaatgttgcggcagggaataaagatttgttgctggcctgactgaacaatcaggccgcaggcttctttggggtattcagcagcgacgtgctgacggatagcatccagcaatttttcacgcat

SEQ ID NO: 157 - DNA, 738 bp, Escherichia coli
  source 1..738: mol_type = genomic DNA
ttatttcccctgcaggtttgcagccggaaaaccgccgaacggcagcggcgcatccgggccgtgacgatcctgacaatcctgccggcggccgccacaaacatctttcgacgggtcatcggtcggtgtaccgtctttggtaaagtatttcgtgccgttgtaatcgcatccggtcccgcttcggtaccagccccgcatacaccaggtgcagacaggcgtaatctgccgtgtcggcagctgcaggctctgaatatcgaaaggagaacacagctcgaaatcaacctgtacccgcgtctctgcggttttagcattgacgtaaaagagctgtaagcgctcatcggccgggctggcacccggattaccgtttttccagttggcggcatcgagatacttcgaaagcgtggtatggattttgaccttagccctgaccatatcgtcatattcaagacacagcgcggtgacatagtttccgacgttcccgacggacagcgtgggcgttggctgggaacctgtactcgataactccatccccttaagttcgtagggatggggatcgtactggtttccctgccagataatggcgggcagattttctgcggcgaaggctgcccacccctcttcctgaatattgtgcgcatgaaaacgcagcacctgatccataccgaattcagtgccgtcgatctcaatcagctgaataacgctgccgggctcaagctgttgtatgtctgccgtaaaactcat

SEQ ID NO: 158 - DNA, 339 bp, Escherichia coli
  source 1..339: mol_type = genomic DNA
tcagggcgcgaacgcctgttcaaaagtgaaggccacagtggcttttttcccggtagggaaagaaacgctgaacgaatcggccttcattctgaacagctttttttcaccccatggagtggtccaccagaacgatttagtaacgtgagacatcaggaaagcgcgcagcgcagccgcctcctgtctggtgcccgtccagtccaggttccacgtttcctgtttgtcgttgatccccatccccgctatctgtttgtagccatccccgaactgggcctgcagcgttcgggctgtttcagtgccctgcgctgtttttcgcgtgcgccaggtaaacgtgtccgtcac

SEQ ID NO: 159 - DNA, 3138 bp, Escherichia coli
  source 1..3138: mol_type = genomic DNA
tcactgtgtcctcctcgaataaagcacgccgcccgcggacatttcttttttcagtcgctcggtgattgtctgctgaacaatcgcctgcagctgtttcgccgtccccgtggcgttcgcctgatttatgcttccgtcactcccctgctggctgatgctgactggggcataaacactgatcccgcccatgccagcaccggctgcgttcccgccgccgaccagaccacccgaggcatacccgcgcatcaggcgatagagattagccacgccgatgcggctggttgattctttggtgaagacgaattccccgcggtgaacgataccggctggctcgtacttgccgccgtgcccggtaaaaccgcccacgtcaaaaccctgtggccggtatgacgggaccgcgaatgactgaccggcagaggaggttttcgccccgccgctaacccagcccattgcactctggatggtgtaagccaccagcagctggttgataacggacacaatcattttaaggatcgagctggtgaattccctgaagctcgccttcccggttgtcgtcaggctggtaagctggcccgccaacccgctgaacgtagcctgagaaatctgctgaacggagctgaaaacgtttgtcgctgaatcctgatattcggcccaaccctgtttcgcaccggccagccagtttgcacgcagggcatcttcagcttcgaacgtcgccctttgctcttccagaaccttttgctgcgcctgagggttgtacgaatagctttcgctgagacgctgcagcgtagtttgtcgcccggcttcccgggtggataacccctcagactgagcctgcaggcccgccctggcggctttttgctgctgctcaaacttcacggcctgatcggccagctggttgagcttttgctggctggcaaccttatcgcccaggtcggccagctgccgcttgtactcgagcgtttcttctttgtgcgccagcagggatttttcctgcgccgtaagctgacgacgcccagcggcctcctgcagaacggtgaactgattttcagtttgccagagatcctgacgctgtttacttatgacgtcgttcacgctggtatgctgctcaagcgttttaagctgggcctgaagggtgagaagttcggcctgcgccttttcctcggctttgtccccggcgggcgttgagtagcttttgcctttcggtgtttttggatccttccactgcttttcaatcccggcgcgggccgcggcaatgtccttttcagtccacagcgtggcgacaccgtctttcgcatcctggcggtttttctcaataagctgactgagctttttctctgctgaagcccgcttttctgccgccgtcgcgccggactccaccagctggttaaactgctgctggctgcggattgcctgagcctgctggtccgttcgcattttttcccgcgcggctgccagcccttcctgggcgtattgctgatcggcaagatcgtaagcctgctttttcagctccacctgctggcgcgcgtttctcagcctttccgcatccgctttctgcagaacgttgttaccggcataatccgggtcgaccttaagattgctggacagcgcgcggtactctttctctgctgcctgccactcagcaaaagagtcctggcgcttcatcgcggtgtcaggattacgcccgacgcccagcatcgcatcccacgcaccggaggcggcattcttcacccagttccaggctttttcgagggatccgagattatcctcgaccgcaccggcgcgctgaatgaccgcgtcggaatatgcccgcatggccagctcggcagccttctgagaatcccccagcgcctgagcagaagctatctgttcatactgggtggctgtcagaaaatgaagggaatcgttgagcgtcgcgaccgcgttaaccggatcatccttcaggcgtttaaactgatttatggtttcgtcaacggcctgcccggtagcctgctgcagcctggcggcaacattgctgaccatgctgacgtcattaccgctgaacgcgccgctgccaacgacctgcgccagcacgcctgcagcggcatgctgagtgatgccattacctgccagcgagcgcgccagcgcctgcagctgccctgacgttttccccgcgtagttcccggtcaggatcagctgcctgttaaattcctcagactctttgctgccgtcgtaccaggccttacccaacccgaataccgccgcggcaatccctccgaccatgctggcgatcccaagaccgcgcagtgacagaagctggtctatccaccctgcccggttagccagcgtgatcccggagccgcgcagcgcgccgaagttaccgcgcatgacctcgccgatcagtattcccagttcctgccgggcggcggcactttgcagccccagaccgtgcgtggcgactttggcagcttcgagcttgcggatatagacttcagccgcatcgctggcaccgacctgcgccgccttcatgcgcagtagctcggtaccggagagcttttgctctgcaacctgttgcttcagctggctgaggaatcgcgtgcgcgctgcggccgatttttcctccacgatctgcagttctttttgacgggccgtggtgcgggaaataagggcgagataatcctgctgggttatgttgccctgtgccctcgctgcgcgaaagcgcgcctgcacgttcgcaagcgactgtgtttcaccattgagctggcgtacgccgtcgatctggcggaaaaatgatgccgcaagttcatcctgtcgacgggcaagcgcagcggcctgcccgtcattctcacgcatgcgctgattaagctcggtcacgcggcggtgagtttcatcaacggactttgaaacgttctgccagtctttggtaagcccttccgttgcggccgactggcgggatttcatatctgcggcagccgccgcgccagcgtcacccacggttttaaacgcagccgcctgccgctctgaagcgcgctgcattcgcgtctggactttttcagagtcctcagccatcccggttagctggccctttatgcgggcaacctgctcactaaacgtggcgctgtcgacgtcaaggttgatgaccagatcgctaatctgctgggccat

SEQ ID NO: 160 - DNA, 273 bp, Escherichia coli
  source 1..273: mol_type = genomic DNA
ctaatctgctgggccatatcggatacctcctgttatcccctcagctgcggccatcagcgcatcatcatccggctcgtcatcgctgatgacgataccggaaggagaaagcaggctgaaatgtgcgggggtaagttccgggtcgcggaagaaaagagtggagatggaataaagcagctctgagaaatgcgcatcgagctgagcgtcctgaaaataatgctcccggtagaactggtgccagtcgcccagctcagtggaagtcattccagccagcat

SEQ ID NO: 161 - DNA, 432 bp, Escherichia coli
  source 1..432: mol_type = genomic DNA
tcagctcgctggcaagggcttttccgccgcaacgggttctgcgctttcggcctccgctggggcatccggatcggcagctttgtcatcctcaaccggaacgagcatgccggagagcagctttatttccatttctgctttaccgatcgcctccggcggccagccgctaagcacctgctggtaaagcgtctccacatccgtgccagccggatcgttatgccacaaagacatcgcaatcaaacgcgcaccgcagcgaatatttgagccaatcagcctggccgtcatttcctgatcgctgatgccgtcgctgtcagcgctgacggccttttcctctgcggccataaacgtgatgtactcaatacgctgaagcgccgacagctcgaagatggtcagtgattctttttgccaggtgaacttctcttttttcagaaacat

SEQ ID NO: 162 - DNA, 744 bp, Escherichia coli
  source 1..744: mol_type = genomic DNA
ttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcat

SEQ ID NO: 163 - DNA, 402 bp, Escherichia coli
  source 1..402: mol_type = genomic DNA
ttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgaatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcat

SEQ ID NO: 164 - DNA, 573 bp, Escherichia coli
  source 1..573: mol_type = genomic DNA
tcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcac

SEQ ID NO: 165 - DNA, 243 bp, Escherichia coli
  source 1..243: mol_type = genomic DNA
tcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccat

SEQ ID NO: 166 - DNA, 327 bp, Escherichia coli
  source 1..327: mol_type = genomic DNA
ttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccat

SEQ ID NO: 167 - DNA, 1947 bp, Escherichia coli
  source 1..1947: mol_type = genomic DNA
ttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcat

SEQ ID NO: 168 - DNA, 1500 bp, Escherichia coli
  source 1..1500: mol_type = genomic DNA
ttactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcat

SEQ ID NO: 169 - DNA, 216 bp, Escherichia coli
  source 1..216: mol_type = genomic DNA
tcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcac

SEQ ID NO: 170 - DNA, 2103 bp, Escherichia coli
  source 1..2103: mol_type = genomic DNA
tcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccat

SEQ ID NO: 171 - DNA, 489 bp, Escherichia coli
  source 1..489: mol_type = genomic DNA
tcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccat

SEQ ID NO: 172 - DNA, 729 bp, Escherichia coli
  source 1..729: mol_type = genomic DNA
ctatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcat

SEQ ID NO: 173 - DNA, 231 bp, Escherichia coli
  source 1..231: mol_type = genomic DNA
ctatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcat

SEQ ID NO: 174 - DNA, 597 bp, Escherichia coli
  source 1..597: mol_type = genomic DNA
atggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataa

SEQ ID NO: 175 - DNA, 198 bp, Escherichia coli
  source 1..198: mol_type = genomic DNA
ctagtcgtcgagttgcaacacaccgtgatccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcat

SEQ ID NO: 176 - DNA, 480 bp, Escherichia coli
  source 1..480: mol_type = genomic DNA
ctattggttattcgacagtcgcactgattcgtaaatccgctcacacgtcattcctgcccggtagctttcgtcagatcgtccagcataatatcgagctgcttctgcaaggcttccgagcatgtcggcaagcattgctgcgttggctccggctgttttgcttctgacggaagtggcgagatctgcggtgtgctttgcggcgtccatgtgggtagcgagttttgttgcttcggcgcgcagctgcttaacagtggtagccaggccagcagaagtaacggcagcgctcgctgcttgagcttgagcatctttaacggcctcatcccgggcgattgttcgcccttgttcaatcatacgagctgcggtctgtgcattcgcttcctgtgaagattccgcgctatcccggtcagcccactttattttccagctgcggttcgtccactcactgccagcgagaaacgaacctaccaacgcaacaatcacaat

SEQ ID NO: 177 - DNA, 549 bp, Escherichia coli
  source 1..549: mol_type = genomic DNA
tcactggtctatcccccagcacgtcagtgcgctttcctggtcccgccgttctacctgcccataacatccatccttctggcccttggtcaggcggcaatcgcggccaccgtctttaatccaccagcggatagcttcacaggctcctttcgtatcgccagcattaattcgcttatagaacgtagacgggaaacattttccggggccgatgttatatgggcagaaagaagcgatacccgctttctgtggttcggtcagtggtactttgatatttcggtcaacccacgccagcgccttgtcgcgttcaatggcgtttacctgggcgcatttctcagctgacagcttcatgccctgtactactggcttgccatcaaccattgttgcaccacggcaaatggtccagagtccgccgccgtcgcgatatgctgtcaagctgttaccctctttctcatccagaaactgatcgagaatcacgggtgcggaagccccggcaagaatcaaaccaacgaccgctgcgctcaatttattcttcagctttagagacatagccat

SEQ ID NO: 178 - DNA, 279 bp, Escherichia coli
  source 1..279: mol_type = genomic DNA
ttattcttcagctttagagacatagccattgcgccgatcctcccgttctttccagcggaaataccagttcactgcacaggtgattaccgtgcatgcgataccgacaataattgcccagtcgctcaggcttaaccctgcaattctgtcggccaacatccaggacacctcttttgctgttttagctgtttcggcatatgccttcgctgatacaccgcagccggcaagcgtggttcctgatccatatgaaagtctgctgtaaatggtgctcattctggtcat

SEQ ID NO: 179 - DNA, 1053 bp, Escherichia coli
  source 1..1053: mol_type = genomic DNA
ttatgattttttcagtttttccacctcttcggtggtctgtataaacctgtctgcctccagttctacgccgatcgcccgacggccaagttctattgcagctttcacagttgaaccagagcccataaagaaatcggcaacgatatcccccggtctgctgctggcgctaatgatctgtttcagcatgtcggcaggtttttcgcatggatgtttgcctggataaaactgaacaggcttatgtgtccatacgtcggtataaggaacaagagcggaaacagagaagcagcgccgaaggattttgtattcctccagcaattctgaatacttgcggtttaatgactggtaggtagccaccagctggtggtgaggatgttcaagcttttgctgaatatgtttatcgatggcgatccgcgtgaacagttcctgcaattttcgatagtccacttcattcggtagttgccattggcttgcaccaaaccagtgtgacgccatgtttttctttccggttgcctcagctatttctttcgagctgacacccagtgattcacgggcattacggaagtaatcaatcagcggcgtcataatgtgctgctttagctctgtgcttttcctttcgtaaacatcctctttacctgtatacggtccaagatagtgctcagcaaacaaaatccgttccgtagatggaaagtacgcacgcaggctttctttattacatccattccagcggcccgatggttttgcccaaatgatgtgattcaaaacgttgaatcgggcgcgcatcataatctctatatctgaggccagtcggtgaccgcaaaacaggtagatgctgccagcaggtttaagaacgcgagcatactcagccaggcagctatcaagccagcgtaagtagtcctcgtcccccttccattggttgtcccagccgttgggcttcactttgaagtacggaggatccgtaactataagatcaatagagttatccgggagggtggcgacgtaatgcagactatcagcgttgattaactcaacactgtttatttttacagtatttttcat

SEQ ID NO: 180 - DNA, 192 bp, Escherichia coli
  source 1..192: mol_type = genomic DNA
ttactggaggcgtttataacatccgaactggtaatcagataaccccgccatcaccagctgcgtaagtatgagctggcaacgttcgtggctgaggtgggtattctgtgcaatctccccagccgttgctggtttatcgcttaattcattgaaaacagcctttgccgtttctgtcatatcttcctgatttagcat

SEQ ID NO: 181 - DNA, 900 bp, Escherichia coli
  source 1..900: mol_type = genomic DNA
ttagcaaatattccacatcatcgtactaccgttatggttttcgataatttttgcggctgggctagtaccaaaagagtgcatatagcaatgatgaatagtaaggaccagatcctgcaacgtttggtcactctctagctccatgatatttaaaccaatattttgagctttgtccaaatgaatatgtctggcatgtgcatacgttgcttggtggttgtttaactcatcacatatacgcttagccttagcttcagcgtcagcctgacctgcgaacataccagtacaaagccatttctggacaatttcgttcgcccagagaattgctttttcacactcgccaatcaacgttggatttagtttttggaacgtaaattgccaccattgcagtgcagcagggttggcaaaaatttccgcttttgctctctcatactcctcaataattgcatgagatgataacccattaaactgtggatcaattggccccaagttcgactgtttacctaaaacgatctgctcagcacaacaagcaagcattgtgccacaactcattgaaatcataggtacaatcgctcggatattggttccgaactttgaacgaagataatgaccaattgattctagagctgcgatatcgcctccaggagtatggagtaagatatccaatcccagactcgtatctaacccattgatagcagacataagaccatttttatcatcatctgacatctggatcagatgttgaaacccaggcccccctttttgaaggaagcctgagtaataagaaattacatttcggccagtatgtttcgataaatcacgtaagtacttgtggcgaacctcatccgctggtgtacgttgagcgatagtacccatctcacccaatacgtctatccaatttggcat

SEQ ID NO: 182 - DNA, 207 bp, Escherichia coli
  source 1..207: mol_type = genomic DNA
tcagtatgagtacagttggtgagattgctgaccgttctgctcagtagtatttggtgttactgtgctgtatgaatagagcacaccacttctcacattcagatcgttttgctgagcgagaacacgcatagcaaaatgctgtacggattcgcctttttgaaactcttggggttgtatgcccattttttcgtaaaattcagcagcgctcat

SEQ ID NO: 183 - DNA, 690 bp, Escherichia coli
  source 1..690: mol_type = genomic DNA
tcacatttctgccactttgagggcttcttcttcctcatagtattcaagagccatggccaacgcagattcatcaagctgggtaaaagcggcctttaacccagcccagtgccctgaatagacacgcaaccatgtcgaacggtcaacgctaaccatgcgggccaacgctgcaccagcatagtctttataggtttcattatttctggttgcggcaatttcctgccctgccagccataccaggcctatcagtttctttactacgcgctcctgaagggagttatcacccaggcatttctgataagttttccagacgtattcacacatcatcacctggtgcttatagctaaggtcaaaaccgtagcagtaccgcaaccaggcctgctggtatccactaagcgcggacactgccctacgccacggcgcggactcaaattccgcatcttttatcggcggcattggcctgcggcggctgcgtgtttccagcacatacagtggcgcggaaagtgagttaacaaagcgtggccccttctctccttcgagttcgacgagatgaattccacggcgaggggtggcatttttgtctgctggtgggtgttcactgaaagcctcaagctgcccttttgttcccccagacaggtcaggtagcgcgcggcgcaattctattcttacaaaattcaggtcttgttgattcat

SEQ ID NO: 184 - DNA, 996 bp, Escherichia coli
  source 1..996: mol_type = genomic DNA
ttaagctttcgcaattacgccgatcgccagcgcccgatccataaaacgcagtagcagctcaagctgcgtaccatgcttctgctcgaatgccggtacatcggcgtgtaactcgtcgtggcactctctgcacagagggatcacgaagagatcatgggcttttgttgctgtcccccccataccgtgccctacgatatggtgcggatcatctgctggccgtcggcaacactcacagggttgtgttttaacccagcgggtgtacgtctcatttatccagcggcgtcgctttggcctgagcatgaaagattctggagactccggatcaacagagagcgtgaggatcttcttcgccttctcctgcacgaggctggttgcagaagcggaaggcacaatgtcgctttccctcatgaccgagcggatcttatcatccggaaggcgtagccccttgtgcgcaacgctttccggaataacatcagccaggtcgtttctgaccatccaccagcacagttccggaagcgtcaggatatgcgactcgggaaaaccagaatcacgccgaatgacttccagaatccaggataccaggtttcctgccgctatacctgcaagctgttcggtatgctgccccgacaaagtgtgatcgcaatgccagcacaggcgaatacttcctggtgggtgccgcattgttgtgaagttcttgtcgtgccacgatgaatgtggccactggcattcaaaccgattactcaaccattgctcaagggaaggaagcccaccagcacgctgaataacccgatcattctcgaagacctgccgcattaccggatcatcagccagcggctgaatggcggcgggaacagctcctgtactgaatgacgccatttcttctggttcaggctcaagcagaacgcgaccgcgcataaagaggtgcatcagttccgcgccgggacgaaacaacacaatccccatacgatgggcgatctcgggggtaagcagagctctcac

SEQ ID NO: 185 - DNA, 684 bp, Escherichia coli
  source 1..684: mol_type = genomic DNA
tcacgcgacctgccccctggcaatgtgttctgcccacagtccaccaatccagcgcacgcctttcgccgtgaaacgtgcctggctgaatgcatgatttgaggttacggatgtgccggttttcacttcaaaacggcccgcatcaatatgctgatgccgtggggtcatcgttccgccaagacgatacatgatgtcgttctcaaggaggaataaccgcagatcgggctctttggccttaagcagttttgccacctggcggaatgacattgacccactggctgtacagtaccgatcaacaaacgctaccttcggcgccgcggcagccagttcgttagtcaactgctgtttttgttctgcaaggtcagctgcaagacgtagggcttcagagaatgattgaggaatcgtctgctgctgtgcctgctcaagctcctgccagcgatcaaccagacgcgcggtaaactccggcgacagctgcgcgacaacgatataactgtcccgcttccctatcagataaaccgataccgactgattgaggtgatttttaacttcccccattggggggagttcaataacaccgcgctctgccaggcgttcaatggaccgtttaacatggtcatgtcttgattccaccagctcagcaatatcgctgctggacatggttaacgctgttgttgctaactggctcat

SEQ ID NO: 186 - DNA, 387 bp, Escherichia coli
  source 1..387: mol_type = genomic DNA
tcaggcggctgcacccgccggttcatatctgctgattgttatctctacccgacctttcggcacaacgggtccccattccaccagcatgcgcttaatctggctgtcgtcttcccagacacccgcatgcgtcagcgcgtcaaacagggctttgttgtaattatcgatatcccggcggcgcgcatccggcgggtacagagtgatttctaccgctgccagttcagtcgatggcttcgggagacgtcgtaattgctcaatgatcgccacgcaggcagcgctctggtatttacggccagcggcgctaatgaggtgacgaccggccagcggccccttgttaggggcgcgccagtaagtgttcacgctcggaggaaaaggcaggatcagtttcac

SEQ ID NO: 187 - DNA, 1320 bp, Escherichia coli
  source 1..1320: mol_type = genomic DNA
tcacgcggcctctccccgcatattgcgaacaagttcagaagcagcagtaatgatttcgctggtggcagtccgctccagccagagttgattgatattggctttcagcttgtgttgcagtgactcatccagcatgtcagcaccatccacctggtcgaatacaattctaacctccagcggccagatacgggactcgggaagcggatccgatactggtttagctttctcacgaatgtgcatgcggatctggcgaatattggaccaactggaaacatccaggcttcccatggctgcaatgaagtcagtgctgttcatgccatattcaccggatgcttcaagggcaacagtgcgaatacgttccgacatatccaggcgcacagcagcgtcatcgaattcaatcgacaacagccactcatccacaccgaacaaaatactctcacgaataagcagcttcgctttgtcgatcgttaaaggtgatacctgagtgaattccggtgcttcgacagaatccgccgcccaggtatgcccaaacttcgattcactgaatgtgtattcttctttatcgccaaacgcagctctaacgcatgcccacgcctcgacaccgctgatagcaaaaatatctttctgggtgagtggcaactctgcttctggcttatcagctgcaggaggtgtggcagttacaggttgagacttgctggcagcaaattgtgccaaagccataaacgcccgcccttttgcctccagttctgtgcggttgatatagctgaaccgctcgccacgccatgacttatcgaatacagctatggcaccggcaaaaaacgcgctggtgggtttctgtttttcgtcagcaggtacaaaccacacaggcagatcgaacccaatgcgcccgcgaatgaatacaatgtgatcggcatcttccggccaccacgtttcactcggcgcggcttttatcaggaatacatagcgaccgcccttctcgcgctgggctgctgcgtagttcatgatgtgcgtcatgccggtgatcgcctgcttctcgtggtactgcgaacggctatacggtgggttgccatagccagcgccacccagttcagccagacgttcagaccagtcctgcgtcagcgcgttatcttcggcggtgtaccatgccgggcatttcgcgttgtcgtcgtcagcaaacaaatccagaactaatggaccaaatagcgcgttgatcccccaaaaaagcagatccggtgtccgccactgatcgccaacctctttcaattcgtgagctggtttgctacgcagtgccgccagcgcctggcaatatttattggtcatcat

SEQ ID NO: 188 - DNA, 882 bp, Escherichia coli
  source 1..882: mol_type = genomic DNA
tcatgaacggaaccccgaattttctggcagtgagtaatcaacactctggaagtttgcgcggctggctgagttagtctcccatttgccgttaacgcgttcaggccggccagcactggaccatttggtcgcgctttgcaggtaaccagggaagttttttggaatgaacagagttgccgggcggaggtattgcgcctgctcgctatcacgccaatcggcatttttgtaatccactaccaagcacaggtcatcaacagtgaattgttcccgaagacgggcgcgaatattctccagcgacgtgctgcatacctggtagcgtgagccagtagtctgattcaggtaagacaaaacctgtctggcctgatcagtaatcacaacctcagggtcgggttgcgccgcaaccggacaagagggttttgaagttacttgtggttcttgttttgattttactgacggatccccaccagattctgacgggtcaaaaccgccttttttgctggatttcgacgcctcaaattttgacgggtcggattttgatgcatcagattttgatgggtcagattttgatgcgtcagattctgacaggtgagaaaaagcagcctctcgcaacttaacgacgttaagctgatatacgttcgatgcgttacggttcccattacggcgctgcttacgggaaagccagccatccttctcaagctgagcaagggccgtccttaccgtactttctccagcaccgatttgacgtgcgatcgtgccaatagacggccagctaacaccctcatcactactgaagtcggccagacgcgccatgatggcaacgctggataacttcatgcctgacgaagcgcatgcatcccatacgtaaccggttaatttagtgctcat

SEQ ID NO: 189 - DNA, 339 bp, Escherichia coli
  source 1..339: mol_type = genomic DNA
ttaattctgtaaatttacgctggaattgttcaagagggctgaagcactcatgatcgtacccttcgcgaaggtatataacgcgctgtgtatctggctcccagcggacaactctgacgggaactccgtagtgatctctgaaccgccggttaacttcagccattcctcgcgccccttctcgttcatctgaacaaatgcttctaccatcaagtctgctggctggtagttgcctccatcagccgcgttatttatgatttccacatagccgaactgggcatctttacccaccagcggcaaacatctgaattgcttagctggtctgaatcggtttacactgttcat

SEQ ID NO: 190 - DNA, 564 bp, Escherichia coli
  source 1..564: mol_type = genomic DNA
tcatgcgttagtttctccactgatacgacacgccaaggcgcccggagctgcacactcgcgggcgtcaccttttctgcctgttgaaacgaatacgtcaatcgcctgatctgaaacaccaaccccataaagcgccataaatcccaggaacccgtgaatctggtggcggagcttcttactgaataattctgaaagcattttgcgctctgatgaatcaattaccccatcagccgctgctgccatcttggcattagccagctcaccagatgctgctgccgctttcatctcaatgctgtacagctcaacgttatccaggctctcagcagttggaacatccaccagccatttcccttttcggttcgcctggtactcggccaagtaacaagaaccagacaggtcctccatccgttccagttctgccaaggtaaagaaccgactgccacacttctggtacaggtggttgtggaactggtcgatagtcatccctaaatcggaagccatacctaagcgaccatgcttgtgtgccttacacatcaggcggattgctgtatttatgctgtctaccat

SEQ ID NO: 191 - DNA, 258 bp, Escherichia coli
  source 1..258: mol_type = genomic DNA
ttaaagttgactattgttgttagcggaaggtatgccgtcatttttgttcggataaatatcaggtcgtaattgatggggagttactacccatccgccccattggcagagttgaataactctttcagaaggtactcggttctttgcaatccagttcgcaacagattgaactgattggaattcaaaccgccttgatacctctgaaatcgacccgatcgccttcacagctttagctgttacattcttgtgttgagatgacat

SEQ ID NO: 192 - DNA, 711 bp, Escherichia coli
  source 1..711: mol_type = genomic DNA
atgccttgtgcgcttaatcttctacttatggtggaaaatgctaaatacaaagactttgccgaaaggctaaacaggtctctccaagagcaatctattggagttaaagaattgtcagagttcagtggtgtctcgtatgagatggcgcggcgctacactcttggtactgcaaagccgagagatgagaagatgattcgaattgcagaaagacttgccgtctcaccggcttatcttgattatggtgtgcctgttaatggtggcgacgcgccagccaaaggcacggtcagaatagagcaattggatgttcatgcttcagccggttccggatatataaaccaaccattccctacaatagtgagctcaatagagattccagaagagaggatcttcgagttgtttggtcgtagaagccttgatggcatcgtcatgataaatgttgatggcgatagcatgatgcccacgctttgcccaaaggacctgcttttcatagacagcaaggttgaacaattcagcggcgacggcgtttatgtgttcaattttgaagacagtacgttcgttaaacgtttgcagaaggtaaaagggcgccgactggcagttctttcagacaatgaacattacccgcccttcttcatagaggagcatgaaatgaatgaactatacatattcggcaagctaatcagatgcttacctctaaaaatgatagagtttggctaa

SEQ ID NO: 193 - DNA, 198 bp, Escherichia coli
  source 1..198: mol_type = genomic DNA
ctatttaaagagcttcttcagcttgtcctcaaccttcctgatttcggaagtaagctggctgctgttgacattgatagtagctccacatcgacaagtgaaacttttgttcgacttgagccaagcgattttcttcttcgtcttagtgccgcacttagggcatgcgggtaacgtaatttcctggttatcaaaagcgcccat

SEQ ID NO: 194 - DNA, 918 bp, Escherichia coli
  source 1..918: mol_type = genomic DNA
atgaataatccgtttttcaaaaatatgttggtgtatcgcattagtcgcgatttcaccatcaaccaggaagagctggaacagcagcttgaactatttcgcttcactccatgcggtagccaggatatggcaaaaaccggttgggtatcaccacttggtcagctgtcagatcgcttgcatcacactgtcaataatcaagtgttgttggttattcgccgggaagaaaaaatactgccatctcctgtcattactgaagaactgcgcaagcgtgtgtcgcgtctagaatccgatcaggggcgtcgcctcaaaaaaactgagaaagattcgctgcgtgatgaagtgttgcactccctgcttcctcgggcgttctccaaaaactcgactgttggtttgtggatcaacgtcaccgacggtctgatcatggttgatgcagccagcgctaaacgtgccgaagactcactggccctgcttcgtaaaactctcggttctctcccggtggtaccgctgactatggaaacgccgatcgaactaactatgaccgactgggttcgttccggtagtgcgcctgctggctttggcctgggtgatgaagccgaactgaaagctattcttgaagatggcggtattggacgctttaaaaaacagactctggtcagtgacgaaattcatgtgcatctggaagctggcaaagtagttacaaagctgtctatcgactggcaacagcgcattcagttcgttctttgcgatgacggcagcatcaaacgccttaagttctctaatgagattacagaacaaaacgacgatatcgaccgtgaggatgcggctcagcggttcgacgctgactttgttctgatgaccggcgagcttatctctctcattaacggattaacaacctctctcggcggcgaagccaagcgataa

SEQ ID NO: 195 - DNA, 540 bp, Escherichia coli
  source 1..540: mol_type = genomic DNA
atgagctacattcagacattatccggcaaacattttaattacctcgatatccaacaggacgatatcgtgatcgaggatattgctaccgcgttgtctcatatctgccgctttgcagggcatcttcctgagttttacagtgtcggccagcatagcgttttaaccagccacctcgttccgcaggagtttgcattagaagcactgcttcatgatgctgctgaagcctacctgcaggacatcccctccccacttaagcgcctgcttccggattaccaggcaatcgaagctcgtgtggacgcagccattcggcagaagttcggtctaccaactgagcaacacccaaccgtgaaatatgccgacctggtgatgctcgccagcgaacgccgcgattttgagattgacgaaggttccatttggccatgcctcgagggagttgtcccaacggatttattcattatcaacccagttcgtcctggccagtcatacggcatgttcatcaatcgctttaacgagttgatggagcagcgccaatgcgccgcatga

SEQ ID NO: 196 - DNA, 255 bp, Escherichia coli
  source 1..255: mol_type = genomic DNA
atgaccgtatttgaatatctccaggctcatccgaataccaccagcggtgaaatcgccaaaggtatgaacaaaaagaccccagcggtcgccggagcattatctcagctctatggcaccggtcggatcgtgaagtctggtgttcgcaagggtattccaacataccgcattaacgatatgccgtttggttgcagtaacagcctaaccatgatgtttaaccagctcttgagcagagccagacaaggagcagcccaatga

SEQ ID NO: 197 - DNA, 348 bp, Escherichia coli
  source 1..348: mol_type = genomic DNA
atgacagcactcaacaaacaggcgctgcgtgaagaattccagttcatgcaggacaactatagcgacccggcagaccacgatcggcaggtgatttacatcgaggcggaggcgctgctggatgagttggaagccaaagactcaacgatagcagcacaacaacatgagatccgtatgttgctgaatgcgcttgaggaaaaaccatgcccgaaatgcaacgacacaggaatgactgatagtggcggcacgcagccatggggcgagccgattgagattgaatgcgactgccgacagcaggatgccaacaccgcagaacttgtagccgctggcattggcgtgaagggggagtga

SEQ ID NO: 198 - DNA, 309 bp, Escherichia coli
  source 1..309: mol_type = genomic DNA
atggataaattaatcaaacctaccgccaaaggtaaatatgacggttcatgtgattatctttgctcggaagatgcgcgattcatcgttatgcgcggcgattatacggaagcggaaataattcaggcttctgtgtctcaagatgtaatcgactcggatggtgcggctgattttgcaagtagcgcccgctattatcagtgctggtacaaagttagcccaataggtggtcaggatggctattcaggctggcatcatcctcgtgattcgccgtgtcgcggtgcatatttcgcatcagttttgcaatgggattaa

SEQ ID NO: 199 - DNA, 468 bp, Escherichia coli
  source 1..468: mol_type = genomic DNA
atgacaactaacaaccacccggcgcacggtcctgtatcactcgatcgcctgcaccagatacgcgaacacctgctgcatgatacccaatactcaaacggcgggaacagagcctacattctcgctgatgtattgaaggtgattgatggggctattgcccgcgagctggtacgccgtgagcatgcagcgtggtcacaggctactttcggcgatgtcggtccagttggtccgctgaagcacctttccaaagaagcgctcgaggctgctgctgaaccaggcgaccttagcgaatgggctgacatgcaattcctgttatgggatgcgcaacgtcgtgccggtatcagtgatgagcagattacccaggcaatgataaaaaagctggctataaataaggttcgccaatggcctgagccgaaagacggggaacctcgattgcatatcaaagaacagtcagagcaggagaaaaaataa

SEQ ID NO: 200 - DNA, 255 bp, Escherichia coli
  source 1..255: mol_type = genomic DNA
atgtttagcctgattcggcgcggtcaaatctacacggacagtagcaactggcccgtaattatccatagctgtagtgatcactcggtccgaattaaacgcaatgatggcgagctgagaacgattagcatcaaacgctttaacgaagattttgaacgagtggagcatgatgagtatcgcaaaatatgtgccgaaatagagcaggaaacaaacctgaaaaacctacgtgcgatgcgtcgcggcaagattactgaatag

SEQ ID NO: 201 - DNA, 570 bp, Escherichia coli
  source 1..570: mol_type = genomic DNA
gtgaacaacttaatgatcgaccttgagtccatgggcaaaaaaccgaatgcccctattgtctccattggtgccgtattcttcgatccgcaaagcggtgaactgggtcaggagttttacaccgctgttaatcttgaaagcgctatggagcagggagcggtgccggatggtgacactattctgtggtggttaagacaaagctcagaagcacgatcagcaatctgtgttgatgatgcgatgccgatatcatctgccctatctgaactgagccatttcattaatcggcattctgataaccctaaatatttaaaagtttggggcaatggagctactttcgacaacgttatattgcgcggcgcatatgagcgtgccggccaggtttgcccgtggcaattttggaatgatcacgacgtcagaaccatcgtcacattaggcagatctgtaggtttcgatcctaagcgtgatatgccatttgatggggttgcacataacgcactggctgatgcccgccaccaggcgaaatatgtttcagcgatttggcagaaactaatcccaaccaccagcaacagctaa

SEQ ID NO: 202 - DNA, 237 bp, Escherichia coli
  source 1..237: mol_type = genomic DNA
atgagcaatattttccagttagctcccaacgattgggtttgtgaaagcgttttgatcgcggttactgggctcaaacccggaaccatcctccgtgccagaaaagaatgctggatgattgggagggagtatatccacgtatcgcctgacggaaatcctaaaccttccagtgagtgcatgtataacagaaaggctgtagatgcctgggtcgcttcaatgaaaagcaagcaaccagggtga

SEQ ID NO: 203 - DNA, 1314 bp, Escherichia coli
  source 1..1314: mol_type = genomic DNA
atggataaagtcacatatccaacaggcgtcgaaaaccacggtggcacattacgcatctggtttaattttaaaggtaagcgtgtcagggaaagtctcggtgtccctgacaccgctaagaacaggaagatagccggggaactgcggacatcagtatgttttgccatccgcacaggaacctttgattatgcaacccagtttcctgactcccctaacctcaaggcttttggtgtaagtaaaaaagacattacagtgaaagaacttgaagaaaaatggctggatctgaaacggatggaaatctgcgcgaacgcatttaatcgctatgaatctgtcgcaaggaatatggtgccgaggatcggaggtaatcgcctggtgtcagcagtaaccaaagaggaattgctgtatctgaggaaatatttgctaactggttatcagaatccgacgaaaaacaaagccccggcaaaagggcgaagcgttgttactgtgaactattacatgacgacaatggccggaatgtttcagtttgctgcggatcacggttacttagaggtgaacccattcgagggaattaagcctctgaaaaaagccagggcagaaccagatcctctgtctcgtgatgaatttattcgcctgatagatgcatgccggcatcagcagacgaaaaacctgtggtcattagcagtgtacacaggaatgcgtcacggggaactggtctccctggcctgggaagatatcgacctgaaggctggaacaattaccgtcagacgtaattatacgaaacttggtgagttcactctaccgaaaaccgaggcaagcacagatcgagtggtgcatcttatccagcccgcaatcagtatcctgaaaaatcaggctgaaatgacaaggctgggcaggcaatatcacattgaagtgcagttacgtgagtacggccgttcggtgaaccatgagtgtacattcgtctttaatccgcatgtggtcagacgcagtaagcaggtcggatttatctaccgggtcgattcagtaggcgactcatgggaagcggcacttaagcgtgcggggatcagacacagaaaggcgtaccagtcacgacacacctatgcgtgctggtcattatcagctggtgcaaaccctagttttattgccagtcagatggggcatgcgagcgcgcagatggtgttcaatgtttacggtgcatggatggctgacagcagcgcagagcagatcgcaatgctgaatcagaagctggcagattttgccccattgatgccccatagccacgagaacagtacgggaggattattaaaatcagtaagttaa

SEQ ID NO: 204 - DNA, 726 bp, Escherichia coli
  source 1..726: mol_type = genomic DNA
gtggagggtaacaccacgctttatgccctgccgaaacccgaggttgtcctgcgctggcgtgagcagaccacagatgacttccgcttctgttttaagtttccggcgaccatttcgcatcaggcagcattacggcattgcgatgatttagtgactgaatttttgacccgcatgtcaccgttggctccgcgcattggacaatactggctgcaactgcctgccacattcggcccacgggagctgcctgcgctttggcattttctcgattctcttcccggtgaatttaattatggggtggaagtccgccatccacagtttttcgccaaaggggaagaggaacaaacgcttaatcgcggtttacatcagcgcggcgttaatcgggtgattttagacagccgcccggttcatgcagcacgtccatacagtgaagctattcgcgacgctcaacgaaaaaaacctaaagttccggtacatgctgtactgacggcgaaaaatccactgatccgttttatcggtagtgatgatatgacgcaaaaccgggaattatttcaggtctggttacaaaaattagcgcagtggcatcagaccactacgccttatctttttttacatacgccagatattgcccaggccccggaactggtacataccctgtgggaagacttacgtaaaacgcttccagagatcggagcagttccggctattccacagcaatcttctcttttctga

SEQ ID NO: 205 - DNA, 396 bp, Escherichia coli
  source 1..396: mol_type = genomic DNA
atggtaagcgcgctgtatgccgttttaagtgcgttgttattaatgaagttctcttttgatgtcgttcgcctgcgaatgcagtaccgcgttgcctatggcgacggcggttttagcgaactgcaaagcgctattcgcattcatggtaacgcggtggaatatattcctatcgcgattgtgttgatgctgtttatggaaatgaatggcgcagaaacctggatggtgcatatttgcggcatcgttttgcttgctggtcgtctgatgcattattacggttttcatcaccgtctgttccgctggcgacgttctggcatgagcgccacctggtgtgcgctgttgctgatggtgctggcgaatctttggtatatgccctgggagttggttttctccctgcgttag

SEQ ID NO: 206 - DNA, 744 bp, Escherichia coli
  source 1..744: mol_type = genomic DNA
atgtctcaccgcgacacgctattttctgcccctatcgccagactgggcgactggacctttgatgaacgggtagctgaagtcttcccggatatgatccagcgttccgttcccggctattccaatattatttccatgattggtatgttagccgagcgcttcgttcaacctggtacgcaggtttacgatctgggttgttctctgggcgcggcgacgctctcggtgcgtcgcaacattcatcatgataattgcaaaattattgccatcgacaactccccggcgatgattgaacgctgccgtcgtcatattgacgcctataaagcccctacgccagtagacgttattgaaggtgatattcgcgatatcgccattgaaaacgcatcgatggtggtgctgaattttaccctgcaattcctggaaccttccgagcgccaggcgttactggataaaatttatcaagggctgaacccgggcggtgcgctggtgctttcggaaaaattcagtttcgaagatgccaaagttggtgaactgctgttcaacatgcaccacgactttaaacgtgccaacggttacagcgaactggagatcagccagaaacgcagcatgctggaaaacgtgatgctgaccgattccgtggaaacccataaagcacgcctgcataaagccggttttgagcatagcgagctgtggttccagtgctttaactttggttcactggtggcattaaaagcagaggacgctgcatga

SEQ ID NO: 207 - DNA, 972 bp, Escherichia coli
  source 1..972: mol_type = genomic DNA
atgatcgactttggtaacttttattctctgattgccaaaaatcatctttcacactggctcgaaacgctgcccgcgcagattgctaactggcagcgcgagcagcagcacgggctgtttaagcagtggtccaacgcggtggaatttctgcctgaaattaaaccgtatcgtctggatttattgcatagcgtaaccgccgaaagcgaagagccactgagcgccgggcaaattaagcgcattgaaacgctgatgcgcaacctgatgccgtggcgcaaagggccgttctcactgtatggcgtcaacatcgataccgaatggcgttccgactggaaatgggatcgcgttatgccccatctttctgatttaaccgggcgcaccattcttgatgtcggctgtggcagcggttatcacatgtggcgcatgattggcgcaggggcgcatctggcggtgggtatcgatcccacgcagctattcctctgccagtttgaagcagtgcgtaaactgctgggtaacgatcagcgcgcacatttgttaccgttaggtattgaacaacttccggcactgaaagcctttgataccgtcttttcgatgggcgtgctttatcatcgtcgttcaccgctggagcatctctggcagttaaaagaccaactggtgaatgaaggcgaactggtgctggaaacgctggttattgatggcgacgaaaacacggtgctggtgccgggcgatcgttacgctcaaatgcgtaatgtctatttcattccttccgcgctggcgctgaaaaactggctgaagaagtgtggttttgttgatattcgcattgcagatgtgagcgttaccaccacagaagagcagcgacgcaccgaatggatggtcaccgagtctctggccgattttctcgacccgcatgatccgggtaaaacggtggaaggttatcctgcgcctaaacgcgcggtgctgattgcgcgcaagccgtaa

SEQ ID NO: 208 - protein, 323 aa, Escherichia coli
METKKNNSEYIPEFDKSFRHPRYWGAWLGVAAMAGIALTPPKFRDPILARLGRFAGRLGKSSRRRALINLSLCFPERSEAEREAIVDEMFATAPQAMVMMAELAIRGPEKIQPRVDWQGLEIIEEIRRNNEKVIFLVPHGWAVDIPAMLMASQGQKMAAMFHNQGNPVFDYVWNTVRRRFGGRLHARNDGIKPFIQSVRQGYWGYYLPDQDHGPEHSEFVDFFATYKATLPAIGRLMKVCRARVVPLFPIYDGKTHRLTIQVRPPMDDLLEADDHTIARRMNEEVEIFVGPRPEQYTWILKLLKTRKPGEIQPYKRKDLYPIK

SEQ ID NO: 209 - protein, 440 aa, Escherichia coli [sequence omitted]

SEQ ID NO: 210 - protein, 310 aa, Escherichia coli
MLHKKTLLFAALSAALWGGATQAADAAVVASLKPVGFIASAIADGVTETEVLLPDGASEHDYSLRPSDVKRLQNADLVVWVGPEMEAFMQKPVSKLPEAKQVTIAQLENVKPLLMKSIHGDDDDHDHAEKSDEDHHHGDFNMHLWLSPEIARATAVAIHGKLVELMPQSRAKLDANLKDFEAQLASTEKQVGNELAPLKGKGYFVFHDAYGYFEKQFGLTPLGHFTVNPEIQPGAQRLHEIRTQLVEQKATCVFAEPQFRPAVVESVARGTSVRMGTLDPLGTNIKLGKTSYSEFLNQLANQYASCLKGD

SEQ ID NO: 211 - protein, 251 aa, Escherichia coli
MTSLVSLENVSVSFGQRRVLSDVSLELKPGKILTLLGPNGAGKSTLVRVVLGLVTPDEGVIKRNGKLRIGYVPQKLYLDTTLPLTVNRFLRLRPGTHKEDILPALKRVQAGHLINAPMQKLSGGETQRVLLARALLNRPQLLVLDEPTQGVDVNGQVALYDLIDQLRRELDCGVLMVSHDLHLVMAKTDEVLCLNHHICCSGTPEVVSLHPEFISMFGPRGAEQLGIYRHHHNHRHDLQGRIVLRRGNDRS

SEQ ID NO: 212 - protein, 261 aa, Escherichia coli
MIELLFPGWLAGIMLACAAGPLGSFVVWRRMSYFGDTLAHASLLGVAFGLLLDVNPFYAVIAVTLLLAGGLVWLEKRPQLAIDTLLGIMAHSALSLGLVVVSLMSNIRVDLMAYLFGDLLAVTPEDLISIAIGVVIVVAILFWQWRNLLSMTISPDLAFVDGVKLQRVKLLLMLVTALTIGVAMKFVGALIITSLLIIPAATARRFARTPEQMAGVAVLVGMVAVTGGLTFSAFYDTPAGPSVVLCAALLFIISMMKKQAS

SEQ ID NO: 213 - protein, 336 aa, Escherichia coli
MIEADRLISAGTTLPEDVADRAIRPKLLEEYVGQPQVRSQMEIFIKAAKLRGDALDHLLIFGPPGLGKTTLANIVANEMGVNLRTTSGPVLEKAGDLAAMLTNLEPHDVLFIDEIHRLSPVVEEVLYPAMEDYQLDIMIGEGPAARSIKIDLPPFTLIGATTRAGSLTSPLRDRFGIVQRLEFYQVPDLQYIVSRSARFMGLEMSDDGALEVARRARGTPRIANRLLRRVRDFAEVKHDGTISADIAAQALDMLNVDAEGFDYMDRKLLLAVIDKFFGGPVGLDNLAAAIGEERETIEDVLEPYLIQQGFLQRTPRGRMATVRAWNHFGITPPEMP

SEQ ID NO: 214 - protein, 203 aa, Escherichia coli
MIGRLRGIIIEKQPPLVLIEVGGVGYEVHMPMTCFYELPEAGQEAIVFTHFVVREDAQLLYGFNNKQERTLFKELIKTNGVGPKLALAILSGMSAQQFVNAVEREEVGALVKLPGIGKKTAERLIVEMKDRFKGLHGDLFTPAADLVLTSPASPATDDAEQEAVAALVALGYKPQEASRMVSKIARPDASSETLIREALRAAL

SEQ ID NO: 215 - protein, 200 aa, Escherichia coli
MNINYPAEYEIGDIVFTCISAALFGQISAASNCWSNHVGIIIGHNGEDFLVAESRVPLSTITTLSRFIKRSANQRYAIKRLDAGLTEQQNQRIVEQVPSRLRKIYHTGFKYESSRQFCSKFVFDIYKEALCIPVGEIETFGELLNSNPNAKLTFWKFWFLGSIPWERKTVTPASLWHHPGLVLIHAVGVETPQPELTEAV

SEQ ID NO: 216 - protein, 173 aa, Escherichia coli
MAIILGIDPGSRVTGYGVIRQVGRQLSYLGSGCIRTKVDDLPSRLKLIYAGVTEIITQFQPDYFAIEQVFMAKNADSALKLGQARGVAIVAAVNQELPVFEYAARQVKQTVVGIGSAEKSQVQHMVRTLLKLPANPQADAADALAIAITHCHVSQNAMQMSESRLNLARGRLR

SEQ ID NO: 217 - protein, 246 aa, Escherichia coli
MAGHSKWANTRHRKAAQDAKRGKIFTKIIRELVTAAKLGGGDPDANPRLRAAIDKALSNNMTRDTLNRAIARGVGGDDDANMETIIYEGYGPGGTAIMIECLSDNRNRTVAEVRHAFSKCGGNLGTDGSVAYLFSKKGVISFEKGDEDTIMEAALEAGAEDVVTYDDGAIDVYTAWEEMGKVRDALEAAGLKADSAEVSMIPSTKADMDAETAPKLMRLIDMLEDCDDVQEVYHNGEISDEVAATL

SEQ ID NO: 218 - protein, 147 aa, Escherichia coli
MAYKRPVSILVVIYAQDTKRVLMLQRRDDPDFWQSVTGSVEEGETAPQAAMREVKEEVTIDVVAEQLTLIDCQRTVEFEIFSHLRHRYAPGVTRNTESWFCLALPHERQIVFTEHLAYKWLDASAAAALTKSWSNRQAIEQFVINAA

SEQ ID NO: 219 - protein, 590 aa, Escherichia coli
MRTEYCGQLRLSHVGQQVTLCGWVNRRRDLGSLIFIDMRDREGIVQVFFDPDRADALKLASELRNEFCIQVTGTVRARDEKNINRDMATGEIEVLASSLTIINRADVLPLDSNHVNTEEARLKYRYLDLRRPEMAQRLKTRAKITSLVRRFMDDHGFLDIETPMLTKATPEGARDYLVPSRVHKGKFYALPQSPQLFKQLLMMSGFDRYYQIVKCFRDEDLRADRQPEFTQIDVETSFMTAPQVREVMEALVRHLWLEVKGVDLGDFPVMTFAEAERRYGSDKPDLRNPMELTDVADLLKSVEFAVFAGPANDPKGRVAALRVPGGASLTRKQIDEYGNFVKIYGAKGLAYIKVNERAKGLEGINSPVAKFLNAEIIEAILERTGAQDGDMIFFGADNKKIVADAMGALRLKVGKDLGLTDESKWAPLWVIDFPMFEDDGEGGLTAMHHPFTSPKDMTAAELKAAPENAVANAYDMVINGYEVGGGSVRIHNGDMQQTVFGILGINEEEQREKFGFLLDALKYGTPPHAGLAFGLDRLTMLLTGTDNIRDVIAFPKTTAAACLMTEAPSFANPTALAELSIQVVKKAENN

SEQ ID NO: 220 - protein, 188 aa, Escherichia coli
MLELNAKTTALVVIDLQEGILPFAGGPHTADEVVNRAGKLAAKFRASGQPVFLVRVGWSADYAEALKQPVDAPSPAKVLPENWWQHPAALGATDSDIEIIKRQWGAFYGTDLELQLRRRGIDTIVLCGISTNIGVESTARNAWELGFNLVIAEDACSAASAEQHNNSINHIYPRIARVRSVEEILNAL

SEQ ID NO: 221 - protein, 129 aa, Escherichia coli
MGFPSPAADYVETRISLDQQLISQPAATYFMRASRSHFREGIIQGALLVVDASLTACDGSLLICAIDGEFRIKRYRTHPQPHLVNLDNGRREALPADDDGYSSAPAIFGVITYIINDARNAEFDDCPVM

SEQ ID NO: 222 - protein, 80 aa, Escherichia coli
MFVELVYDKRNFDGLPGAKDIILGELTKRVHRIFPDADVRVKPMMTLPAINTDASKHEKEQISRTVQEMFEEAEFWLVSE

SEQ ID NO: 223 - protein, 126 aa, Escherichia coli
MLWRIFIFVNVGLGEAAKRNVGTGENQIPDMSAFPSGNNWFQLPSGHIVQIFSMNVLGADANGTSANYPIAFPTTMIAVSALWSDGTVANAPTYKMMGNTTNRTTLTIKVSASSGTYGTMIIAVGR

SEQ ID NO: 224 - protein, 147 aa, Escherichia coli
MNKYSYSPSENAFYAVALKNTYELSGTWPADALDIPDDISVKYMAEPPQGKIRVAGENGFPTWAEIPPPSHEELIEQAESERQLLINQANEYMNSKQWPGKAAIGRLKGEELAQYNSWLDYLDALELVDTSGTPDIEWPTPPAVQAR

SEQ ID NO: 225 - protein, 197 aa, Escherichia coli
MKPVFDENGLAAVPGDMRCFYYDAVTSEYTGWSDEYINTGVSMPACSTGIDPDENIPGRVAVFTGKGWSHEEDHRNETVYSIENGAAVTVDYIGAIKNGYVTLSPLTPYDKWDGEKWVTDTEAQHGASVEAAEAQRQSLIDAAMASISLIQLKLQAGRKLTQAETTRLNAVLDYIDAVTATDTSTAPDVIWPELPEA

SEQ ID NO: 226 - protein, 310 aa, Escherichia coli
MIYSTGTISINGNTATGSGTNWTAPASQVRAGQTIIVMSNPVQLFQISSVNSATSMTVTPAVSPALSGQKYGILVSDNISVDGLAQAMSQLIKEYDENIGAWETFATTSANQSITVTINGTAVTIPGIGKLAQKGSNGAVTVADGGTGATNAADARTNLGLGEGSPAIGVPFFWPSAAMPNTVIDSWSSMVFLKFNGAKFSATDYPVLAKVFPSLVLPEARGDFIRVWDDGRGADGGRELLSWQEATNFSQFAGNIGGGAGHAINFHDGIAGNQPGFSRFNFNSNSVGDGVNFVAVRPRNIAFNFLVRAK

SEQ ID NO: 227 - protein, 1308 aa, Escherichia coli
MGEKMQLLKQETILQGAKGGGGSSHTPVEQPDDLLSVAKLKMLIAVSEGEIQGDLTAQNIFLNDTPLANDSGEYNFSGVKWEFRKGTQDQTYIAGMPQVDNELAVGTTVTTTAPWTRQFTNLSLDAIRIKLSLPVQYLYKDNGDMVGTVTEYAIDLSTDGGAWKTVVNGKFDGKTTTEYQRDHRIDLPKSTSGWSVRVRRITADASGSNSKLVNAFKVFSYAEVIDSKLRYPLTALLYVEVDSSQFNGSAPKVTCKIKGKLIKVPDNYDPKTRTYSGSWSGGFKMAWSNNPAWIFYDLVLDEIYGMGTRVDASMVDKWALYSIAQYCDEMVSDGAGGTEPRFTCNVFIQSQEDAWQVLNDLAAVFRGITFWGNDQIYVQADVPQDDVDWVYNVSNVIDGLFTYAGGSYKNRYSSCLVSWSDPQNHYSDTVEGVYDSALVERYDVRQTSLTAIGCTSQSEAHRRGRWVLLSNAKDGTVSFGVGLDGYIPLPAEIIGVADPFRSGKENGGRISAANGRQITLDREIDYAAKDRLVVNLPDGKAQTRTISAVSADKKTVTVATAFSQVPVAGAVWAIDSDNLAIQYFRVTSIAANDDSTGGFTITAVQHDPNKYRYIDDGVRVESPPITVTPISVLSAPKNIVVTESDHVSQGLTVASLDVSWDKVEGAIRYVAQWRKDNGDWINVPVTSAQGFSVQGIYSGSYDVRVRALNAQDTSSPWGYGETTYLSGKTGKPGTPLNFLATEDVVWHIDLTWKFPDGSGDTAYTEIQRATTADYANPELLVLVPYPAADYQHGPMPAGVRQWYRARLIDRIGNAGDWTDWIMGTSSIDVSEITNDILEDMKESETFKDLIENAVDSNEKIAGMANDIKQANDELEQQAKDIAKNAQDVGKVQTSVNELSSTVGNVSSSLSQLEQTVATADTALGQRIDNISVSMDGMTGGVKNSAIAIIQANLAQVATRKTLSASVAGNSANLDRLDEVIVSEKEATARSLLSLQTDVNGNKASINSLNQTLSDYQQATATQINGITATVNGHTSAITTNAQAIANVNGELSAMYNIKVGVSSNGQYYAAGMGIGVENTPSGMQSQVIFLADRFAVTTAAGNSVALPFVIQNGQTFIRASFIQDGTIENAKIGNYIQSNNYAAGSAGWKLNKAGDAEFNNVTVRGVVYASGGSFTGEIQATSGKFKGTVEAQSFIGDIANMHTGTNVSRSSDGYLEKVMTYNDSSSSGHARHVCVIANVKGNGAGTIDINGNESGSSVQDMERLIMHSAVVTGPNVTVRIKVSAQNNRGASISSPTIIVSHGSGSFTG

SEQ ID NO: 228 - protein, 205 aa, Escherichia coli
MVKTLILEGKMAKKFGKRVQFDVADLREMLRAMCSQVPGFKKYMSEAHMKGIRFAFFNGGNNIGLEEFDMTRGGSVYRIVPVYEGAKSSGVLQIVVGAVALVAAFFTAGASMAAWGAAMSATAISATSILTGVGVSMMLGGVVQMLTPQPSFGAGKSSSTDNTPNYAFGAPVNTVAMGHPVPLAYGLTEAGGAIVSAGMYSSDQQ

SEQ ID NO: 229 - protein, 239 aa, Escherichia coli
MREKLLDAIRQHVAAEYPKEACGLIVQSGQQQIFIPCRNIADKPEETFTLSPEDQLAARARGEIIMLIHSHPDVVRLVPSELDRIQCDWSGIEWGIMSWPDGDFCTISPREDRDYAGRQWVLGYADCWSLIREFYLREYGIVLGNYSVPYEWWESGKERLYDDNWEREGFVEIAAGAMQPGDIIMMSVQASVTNHAAVYVGDNIILHHLFGHLSSRTPYGKYYRDRTVRVVRHKDRMHG

SEQ ID NO: 230 - protein, 245 aa, Escherichia coli
MSFTADIQQLEPGSVIQLIEIDGTEFGMDQVLRFHAHNIQEEGWAAFAAENLPAIIWQGNQYDPHPYELKGMELSSTGSQPTPTLSVGNVGNYVTALCLEYDDMVRAKVKIHTTLSKYLDAANWKNGNPGASPADERLQLFYVNAKTAETRVQVDFELCSPFDIQSLQLPTRQITPVCTWCMRGWYRSGTGCDYNGTKYFTKDGTPTDDPSKDVCGGRRQDCQDRHGPDAPLPFGGFPAANLQGK

SEQ ID NO: 231 - protein, 112 aa, Escherichia coli
MTDTFTWRTRKTAQGTETARTLQAQFGDGYKQIAGMGINDKQETWNLDWTGTRQEAAALRAFLMSHVTKSFWWTTPWGEKKLFRMKADSFSVSFPTGKKATVAFTFEQAFAP

SEQ ID NO: 232 - protein, 1045 aa, Escherichia coli
MAQQISDLVINLDVDSATFSEQVARIKGQLTGMAEDSEKVQTRMQRASERQAAAFKTVGDAGAAAAADMKSRQSAATEGLTKDWQNVSKSVDETHRRVTELNQRMRENDGQAAALARRQDELAASFFRQIDGVRQLNGETQSLANVQARFRAARAQGNITQQDYLALISRTTARQKELQIVEEKSAAARTRFLSQLKQQVAEQKLSGTELLRMKAAQVGASDAAEVYIRKLEAAKVATHGLGLQSAAARQELGILIGEVMRGNFGALRGSGITLANRAGWIDQLLSLRGLGIASMVGGIAAAVFGLGKAWYDGSKESEEFNRQLILTGNYAGKTSGQLQALARSLAGNGITQHAAAGVLAQVVGSGAFSGNDVSMVSNVAARLQQATGQAVDETINQFKRLKDDPVNAVATLNDSLHFLTATQYEQIASAQALGDSQKAAELAMRAYSDAVIQRAGAVEDNLGSLEKAWNWVKNAASGAWDAMLGVGRNPDTAMKRQDSFAEWQAAEKEYRALSSNLKVDPDYAGNNVLQKADAERLRNARQQVELKKQAYDLADQQYAQEGLAAAREKMRTDQQAQAIRSQQQFNQLVESGATAAEKRASAEKKLSQLIEKNRQDAKDGVATLWTEKDIAAARAGIEKQWKDPKTPKGKSYSTPAGDKAEEKAQAELLTLQAQLKTLEQHTSVNDVISKQRQDLWQTENQFTVLQEAAGRRQLTAQEKSLLAHKEETLEYKRQLADLGDKVASQQKLNQLADQAVKFEQQQKAARAGLQAQSEGLSTREAGRQTTLQRLSESYSYNPQAQQKVLEEQRATFEAEDALRANWLAGAKQGWAEYQDSATNVFSSVQQISQATFSGLAGQLTSLTTTGKASFREFTSSILKMIVSVINQLLVAYTIQSAMGWVSGGAKTSSAGQSFAVPSYRPQGFDVGGFTGHGGKYEPAGIVHRGEFVFTKESTSRIGVANLYRLMRGYASGGLVGGGNAAGAGMGGISVYAPVSISQQGSDGSINQANATGTAKQLQAIVQQTITERLKKEMSAGGVLYSRRTQ

SEQ ID NO: 233 - protein, 90 aa, Escherichia coli
MLAGMTSTELGDWHQFYREHYFQDAQLDAHFSELLYSISTLFFRDPELTPAHFSLLSPSGIVISDDEPDDDALMAAAEGITGGIRYGPAD

SEQ ID NO: 234 - protein, 143 aa, Escherichia coli
MFLKKEKFTWQKESLTIFELSALQRIEYITFMAAEEKAVSADSDGISDQEMTARLIGSNIRCGARLIAMSLWHNDPAGTDVETLYQQVLSGWPPEAIGKAEMEIKLLSGMLVPVEDDKAADPDAPAEAESAEPVAAEKPLPAS

SEQ ID NO: 235 - protein, 247 aa, Escherichia coli
MPTPNPLAPVKGAGTTLWLYTGTGNAFANPLSDIDWNRLAKIKELTPGEMTAESYDDTYLDDEDADWNATAQGAKSAGDTSFTLAWKPGEEGQKDLVAWFIDGSVRYYKIKYPNGTVDVFRGWCSSLGKAIPAKEVITRTAKITNTGKPELAEESGTPNIPVTGVTLDKATASVAVGATTTLNVTVNPASASDTSFRVATSDGAKATVTVSGNAITVTGVAAGTADVIVMTSDGNFVAVCKVTVTAA

SEQ ID NO: 236 - protein, 133 aa, Escherichia coli
MNRHSAIRAAILAKLKAEITDTVTWFDGRPVFLEEQDLPAVAVYLSDAEYTGDSLDEDSWQAVVHIEVFLKASSPDSALDSWMEEKVYPAMAFIPGLTELVETFTPQGYDYQRDDEMATWGSVDFTYLITYSI

SEQ ID NO: 237 - protein, 190 aa, Escherichia coli
MKGLERAIQNLNSLSRLIVPEATAKALNRVARRTISQGSKAVAKEATVDDNRKKGLPVRLVRQRSRLRKARHDRPVASIKINRGNLPAIKLGTARVRLSRKKGARNGAGSVLKIGPYTFRNAFIQQLANGRWQVMRRVGQARYPIDVVKVPLETPLTVAFTAISKRLIESDMPKELSAALKNQLRIHLKR

SEQ ID NO: 238 - protein, 80 aa, Escherichia coli
MDAATIKKMGKTAIINGSSYDVVPAEQLEEMGPLSGTGTSLVVFSELYQPRRNDSVDYDGKNLIVTRYDMFNGKPRIHLE

SEQ ID NO: 239 - protein, 108 aa, Escherichia coli
MAKNYAQDGKTIPLVNSGATDVHSGDPVVVGKLIAVAITDIPAGDTGDGFTEGVFLLPKVSADAVTAGAQVYLKDGKITIEETDAVAAGIAWEDAGANTTVVEVKINA

SEQ ID NO: 240 - protein, 648 aa, Escherichia coli
MQASNNSEADIFIYDEIGYWGVTAKQFVNDLRALGDVTHINLYINSPGGDVFDGIAIYNALKHHGAAITVHIDGLAASMASVIAMVGNPVIMPENTMMMIHKPWGFAGGDASDMRDYADLLDKVESVLIPAYAQKTGKSTEEIAVMLEDETWMNGSECLELGFADQVTPSLQAMACIHSKRIEEFEKMPKSIRNMITPPRNTTQRDPVITQPQAPQAKTDPAPDENAIRAQVLAEQKTRVNAIGDLFAMFGNKHMELQNQCVADPDCSVDKAKDLLLAELGKTATPSNKTTQPHIHAGNGNFVADGIRQALMARAGFEGQERDNVYNGMTLREYARMALTEKGIGVASYNPMQMVGLALTHSTSDFGNILLDVANKALIQGWDEAQETFEQWTKKGQLSDFKTAHRVGMGGFPSLRQVREGAEYKYITTSDKGETIALATYGEIFSVTRQAIINDDLNQLTDVPMKMGRAAKATIGDLVYAILTKNPKLSDGKALFHADHKNLSSGAISVASLDESRKLMRLQKEGERTLNIRPAYMLVPVALETLANQTIKSASVKGADINAGIVNPIQNFAEVIAEPRLDEADAKAWYLAAAKGTDTIEVAYLNGVDTPYIDQQEGFTTDGIATKVRIDAGVAPLDYRGMTKSSGQ

SEQ ID NO: 241 - protein, 499 aa, Escherichia coli
MSFLDDAIGLFSPGWKASRLRARAVIKAYEAVKQTRTHKAQKENRSADQLSQMGAVSLRQQARWLDNNHDLVIGVFDKLEERVVGAKGIIVEPHPMLSNGKIAKKLATDIRRKWGEWSVRPDVTTQFTRPMLERLMLRTWLRDGEVFAQLVRGTGNGLQPVAGVPFWLEALEPDFVPMNSDAATQLNQGVFVDNWGRPKKYQVYKSLPVSGRQFDTKEIDAENMLHLKFTRRLHQTRGTSLLSGVLMRLSALKEYEDSELTAARIAAALGMYIKKGDGQSFDSDSSSDDRELMIQPGMLYDELQAGEEIGMIKSDRPNPNLESFRNGQLRAVSAGSRLSFSSTSRNYNGTYSAQRQELVESTDGYLILQDWFIGSVTRPMYRAWLKMAIAAGEIKLPRGIDMDSLYNAVYSGPVMPWIDPVKEANAWKTQIRGGAATESDWIRASGRNPDDVKSRRKAEVDENREQGLVFDTDPANDKGGTSAEAKEPGAPPSESQRKK

SEQ ID NO: 242 - protein, 71 aa, Escherichia coli
MNPADIQNMIDRYAAAELSVLEGKSITFNGQQMTLENLSEIRKGRQEWERRLATLNNKRRGRPGYRLARFG

SEQ ID NO: 243 - protein, 700 aa, Escherichia coli
MAKRASARDIRRDVSGILRAPRRMPVADAVSTYMRVPMGAGNSVPWDPDLAPYVIEPMNCLASREYDAVVFVGPARTGKTIGLIDGWIVYNIVCDPADMLVIQVSEEKAREHSKKRLDRTFRCSPEVKTRLSPRRNDNNVYDRTFRAGNYLKLGWPSVNIMSSSDYKSVALTDYDRFPEDIDGEGDAFSLASKRTTTFMSSGMTLVESSPGRDIRDTKWRRSTPHEAPPTTGILSLYNRGDRRRLYWPCPHCGEYFQPEMDNMTGYRDSSDPVLASEAAFLQCPACKGRITPDMKRALNMKCVWLRDGQTIDRKGLVSGDGRRSRIASFWMEGPAAAYQTWAQLIYKFLTAEQEYESTRSEETLKTVINTDFGRPYLPRASMEQRKSELLEQRAEDVPKRSVPNGVQFLTATVDVQAGRNRRFVVQITGYGSMGERWIVDRYNIRHSLRCDGNGESIQVDPASYPEDWDLLLTDVFDKTWPLAADPSKGMRLMSMAVDSGGEDGVTDNAYKFWRRCRREGLGKRIYLFKGDSVRRSKLIQRTFPDNTGRSTRRAQATGDVPLYLLQTDALKDRVNNALWRDSPGPGYVHFPAWLGSWFYDELTYEERSNEGKWSKPGRGANEAFDLLVYADALAILSGYEKIKWPSAPEWARRETWIEDTQTEAGEMPSPPPAPKSKPKPKREKPVTEQANPWSSSGGWV

SEQ ID NO: 244 - protein, 162 aa, Escherichia coli
MDQEIATLKLNINQLAGITGVHRQTVAARLKNVEPAPGSNSKLKLYLVTDILTELMIPTVSANIDDMPPSDRLSHWKAENERLKFEQDTGQLIPADEVAREFSLMAKAVVMVLETLPDVLERDCALTPAAVVRVQSVIDDLRDQMAEKVQDAGKEEEQPEED

SEQ ID NO: 245 - protein, 242 aa, Escherichia coli
MSNKIITLSGAANEVLYALFFRGALLSGDLPSKSGTAELRELGFAETRHTATEYQKENHFTFLTSEGQKFAVEHLVNTRFGEQQYCASMTLGVEIDTSAAQKAIDELDQRIRDTVSFELIRNGVSFIKDAAIANGAIHAAAIETPQPVTNIYNISLGIQRDEPAQNKVTVSADKFKVKPGVDTNIETLIENALKNAAECAALDVTKQMAADKKAMDELASYVRTAIMMECFPGGVIWQQCRR

SEQ ID NO: 246 - protein, 76 aa, Escherichia coli
MKILAFINPPGSYMQIETKEMVIFLRDDGISLQYPDGQCAGYGIDGNMLIFMGQSCELFPTKIILHDQRTTNFTHK

SEQ ID NO: 247 - protein, 198 aa, Escherichia coli
MDYYHEIDFPSLFARAVESDDDVGTTLRIHLLCERMVEAWICACCDCQDLFGRDKNKLLIECNTKISMAGNLGIPPELMKSLKTINSMRNDLAHNPSIQSIADSRIQSLKDTLTEYFKQHPTEPSMEESKLGIFNAENQLTEEVSLDSDSSKNRLKLILLFSKLMQALMQLVAANHNGRWDNQFSQFVYHVTMNATKR

SEQ ID NO: 248 - protein, 65 aa, Escherichia coli
MTVVLTAKQIEDLAAFAKEDGQPQYTITTGTIPEFEADNGEIIPGYTGLIAYSESLDHGVLQLDD

SEQ ID NO: 249 - protein, 159 aa, Escherichia coli
MVIVALVGSFLAGSEWTNRSWKIKWADRDSAESSQEANAQTAARMIEQGRTIARDEAVKDAQAQAASAAVTSAGLATTVKQLRAEATKLATHMDAAKHTADLATSVRSKTAGANAAMLADMLGSLAEAARYYAGRSDESYRAGMTCERIYESVRLSNNQ

SEQ ID NO: 250 - protein, 182 aa, Escherichia coli
MAMSLKLKNKLSAAVVGLILAGASAPVILDQFLDEKEGNSLTAYRDGGGLWTICRGATMVDGKPVVQGMKLSAEKCAQVNAIERDKALAWVDRNIKVPLTEPQKAGIASFCPYNIGPGKCFPSTFYKRINAGDTKGACEAIRWWIKDGGRDCRLTKGQKDGCYGQVERRDQESALTCWGIDQ

SEQ ID NO: 251 - protein, 92 aa, Escherichia coli
MTRMSTIYSRLSYGSGTTLAGCGVSAKAYAETAKTAKEVSWMLADRIAGLSLSDWAIIVGIACTVITCAVNWYFRWKEREDRRNGYVSKAEE

SEQ ID NO: 252 - protein, 350 aa, Escherichia coli
MKNTVKINSVELINADSLHYVATLPDNSIDLIVTDPPYFKVKPNGWDNQWKGDEDYLRWLDSCLAEYARVLKPAGSIYLFCGHRLASDIEIMMRARFNVLNHIIWAKPSGRWNGCNKESLRAYFPSTERILFAEHYLGPYTGKEDVYERKSTELKQHIMTPLIDYFRNARESLGVSSKEIAEATGKKNMASHWFGASQWQLPNEVDYRKLQELFTRIAIDKHIQQKLEHPHHQLVATYQSLNRKYSELLEEYKILRRCFSVSALVPYTDVWTHKPVQFYPGKHPCEKPADMLKQIISASSRPGDIVADFFMGSGSTVKAAIELGRRAIGVELEADRFIQTTEEVEKLKKS

SEQ ID NO: 253 - protein, 63 aa, Escherichia coli
MLNQEDMTETAKAVFNELSDKPATAGEIAQNTHLSHERCQLILTQLVMAGLSDYQFGCYKRLQ

SEQ ID NO: 254 - protein, 299 aa, Escherichia coli
MPNWIDVLGEMGTIAQRTPADEVRHKYLRDLSKHTGRNVISYYSGFLQKGGPGFQHLIQMSDDDKNGLMSAINGLDTSLGLDILLHTPGGDIAALESIGHYLRSKFGTNIRAIVPMISMSCGTMLACCAEQIVLGKQSNLGPIDPQFNGLSSHAIIEEYERAKAEIFANPAALQWWQFTFQKLNPTLIGECEKAILWANEIVQKWLCTGMFAGQADAEAKAKRICDELNNHQATYAHARHIHLDKAQNIGLNIMELESDQTLQDLVLTIHHCYMHSFGTSPAAKIIENHNGSTMMWNIC

SEQ ID NO: 255 - protein, 68 aa, Escherichia coli
MSAAEFYEKMGIQPQEFQKGESVQHFAMRVLAQQNDLNVRSGVLYSYSTVTPNTTEQNGQQSHQLYSY

SEQ ID NO: 256 - protein, 229 aa, Escherichia coli
MNQQDLNFVRIELRRALPDLSGGTKGQLEAFSEHPPADKNATPRRGIHLVELEGEKGPRFVNSLSAPLYVLETRSRRRPMPPIKDAEFESAPWRRAVSALSGYQQAWLRYCYGFDLSYKHQVMMCEYVWKTYQKCLGDNSLQERVVKKLIGLVWLAGQEIAATRNNETYKDYAGAALARMVSVDRSTWLRVYSGHWAGLKAAFTQLDESALAMALEYYEEEEALKVAEM

SEQ ID NO: 257 - protein, 331 aa, Escherichia coli
MRALLTPEIAHRMGIVLFRPGAELMHLFMRGRVLLEPEPEEMASFSTGAVPAAIQPLADDPVMRQVFENDRVIQRAGGLPSLEQWLSNRFECQWPHSSWHDKNFTTMRHPPGSIRLCWHCDHTLSGQHTEQLAGIAAGNLVSWILEVIRRDSGFPESHILTLPELCWWMVRNDLADVIPESVAHKGLRLPDDKIRSVMRESDIVPSASATSLVQEKAKKILTLSVDPESPESFMLRPKRRRWINETYTRWVKTQPCECCRRPADDPHHIVGHGMGGTATKAHDLFVIPLCRECHDELHADVPAFEQKHGTQLELLLRFMDRALAIGVIAKA

SEQ ID NO: 258 - protein, 227 aa, Escherichia coli
MSQLATTALTMSSSDIAELVESRHDHVKRSIERLAERGVIELPPMGEVKNHLNQSVSVYLIGKRDSYIVVAQLSPEFTARLVDRWQELEQAQQQTIPQSFSEALRLAADLAEQKQQLTNELAAAAPKVAFVDRYCTASGSMSFRQVAKLLKAKEPDLRLFLLENDIMYRLGGTMTPRHQHIDAGRFEVKTGTSVTSNHAFSQARFTAKGVRWIGGLWAEHIARGQVA

SEQ ID NO: 259 - protein, 128 aa, Escherichia coli
MKLILPFPPSVNTYWRAPNKGPLAGRHLISAAGRKYQSAACVAIIEQLRRLPKPSTELAAVEITLYPPDARRRDIDNYNKALFDALTHAGVWEDDSQIKRMLVEWGPVVPKGRVEITISRYEPAGAAA

SEQ ID NO: 260 - protein, 439 aa, Escherichia coli
MMTNKYCQALAALRSKPAHELKEVGDQWRTPDLLFWGINALFGPLVLDLFADDDNAKCPAWYTAEDNALTQDWSERLAELGGAGYGNPPYSRSQYHEKQAITGMTHIMNYAAAQREKGGRYVFLIKAAPSETWWPEDADHIVFIRGRIGFDLPVWFVPADEKQKPTSAFFAGAIAVFDKSWRGERFSYINRTELEAKGRAFMALAQFAASKSQPVTATPPAADKPEAELPLTQKDIFAISGVEAWACVRAAFGDKEEYTFSESKFGHTWAADSVEAPEFTQVSPLTIDKAKLLIRESILFGVDEWLLSIEFDDAAVRLDMSERIRTVALEASGEYGMNSTDFIAAMGSLDVSSWSNIRQIRMHIREKAKPVSDPLPESRIWPLEVRIVFDQVDGADMLDESLQHKLKANINQLWLERTATSEIITAASELVRNMRGEAA

SEQ ID NO: 261 - protein, 293 aa, Escherichia coli
MSTKLTGYVWDACASSGMKLSSVAIMARLADFSSDEGVSWPSIGTIARQIGAGESTVRTALAQLEKDGWLSRKQRRNGNRNASNVYQLNVVKLREAAFSHLSESDASKSDPSKSDASKSDPSKFEASKSSKKGGFDPSESGGDPSVKSKQEPQVTSKPSCPVAAQPDPEVVITDQARQVLSYLNQTTGSRYQVCSTSLENIRARLREQFTVDDLCLVVDYKNADWRDSEQAQYLRPATLFIPKNFPGYLQSATKWSSAGRPERVNGKWETNSASRANFQSVDYSLPENSGFRS

SEQ ID NO: 262 - protein, 112 aa, Escherichia coli
MNSVNRFRPAKQFRCLPLVGKDAQFGYVEIINNAADGGNYQPADLMVEAFVQMNEKGREEWLKLTGGSEITTEFPSELSAGSQIHSALYTFAKGTIMSASALLNNSSVNLQN

SEQ ID NO: 263 - protein, 187 aa, Escherichia coli
MVDSINTAIRLMCKAHKHGRLGMASDLGMTIDQFHNHLYQKCGSRFFTLAELERMEDLSGSCYLAEYQANRKGKWLVDVPTAESLDNVELYSIEMKAAAASGELANAKMAAAADGVIDSSERKMLSELFSKKLRHQIHGFLGFMALYGVGVSDQAIDVFVSTGRKGDARECAAPGALACRISGETNA

SEQ ID NO: 264 - protein, 85 aa, Escherichia coli
MSSQHKNVTAKAVKAIGSISEVSRRFEFQSVQSVANWIAKNRVPSERVIQLCQWGGWVVTPHQLRPDIYPNKNDGIPSANNNSQL

SEQ ID NO: 265 - protein, 236 aa, Escherichia coli
MPCALNLLLMVENAKYKDFAERLNRSLQEQSIGVKELSEFSGVSYEMARRYTLGTAKPRDEKMIRIAERLAVSPAYLDYGVPVNGGDAPAKGTVRIEQLDVHASAGSGYINQPFPTIVSSIEIPEERIFELFGRRSLDGIVMINVDGDSMMPTLCPKDLLFIDSKVEQFSGDGVYVFNFEDSTFVKRLQKVKGRRLAVLSDNEHYPPFFIEEHEMNELYIFGKLIRCLPLKMIEFG

SEQ ID NO: 266 - protein, 65 aa, Escherichia coli
MGAFDNQEITLPACPKCGTKTKKKIAWLKSNKSFTCRCGATINVNSSQLTSEIRKVEDKLKKLFK

SEQ ID NO: 267 - protein, 305 aa, Escherichia coli
MNNPFFKNMLVYRISRDFTINQEELEQQLELFRFTPCGSQDMAKTGWVSPLGQLSDRLHHTVNNQVLLVIRREEKILPSPVITEELRKRVSRLESDQGRRLKKTEKDSLRDEVLHSLLPRAFSKNSTVGLWINVTDGLIMVDAASAKRAEDSLALLRKTLGSLPVVPLTMETPIELTMTDWVRSGSAPAGFGLGDEAELKAILEDGGIGRFKKQTLVSDEIHVHLEAGKVVTKLSIDWQQRIQFVLCDDGSIKRLKFSNEITEQNDDIDREDAAQRFDADFVLMTGELISLINGLTTSLGGEAKR

SEQ ID NO: 268 - protein, 179 aa, Escherichia coli
MSYIQTLSGKHFNYLDIQQDDIVIEDIATALSHICRFAGHLPEFYSVGQHSVLTSHLVPQEFALEALLHDAAEAYLQDIPSPLKRLLPDYQAIEARVDAAIRQKFGLPTEQHPTVKYADLVMLASERRDFEIDEGSIWPCLEGVVPTDLFIINPVRPGQSYGMFINRFNELMEQRQCAA

SEQ ID NO: 269 - protein, 84 aa, Escherichia coli
MTVFEYLQAHPNTTSGEIAKGMNKKTPAVAGALSQLYGTGRIVKSGVRKGIPTYRINDMPFGCSNSLTMMFNQLLSRARQGAAQ

SEQ ID NO: 270 - protein, 115 aa, Escherichia coli
MTALNKQALREEFQFMQDNYSDPADHDRQVIYIEAEALLDELEAKDSTIAAQQHEIRMLLNALEEKPCPKCNDTGMTDSGGTQPWGEPIEIECDCRQQDANTAELVAAGIGVKGE

SEQ ID NO: 271 - protein, 102 aa, Escherichia coli
MDKLIKPTAKGKYDGSCDYLCSEDARFIVMRGDYTEAEIIQASVSQDVIDSDGAADFASSARYYQCWYKVSPIGGQDGYSGWHHPRDSPCRGAYFASVLQWD

SEQ ID NO: 272 - protein, 155 aa, Escherichia coli
MTTNNHPAHGPVSLDRLHQIREHLLHDTQYSNGGNRAYILADVLKVIDGAIARELVRREHAAWSQATFGDVGPVGPLKHLSKEALEAAAEPGDLSEWADMQFLLWDAQRRAGISDEQITQAMIKKLAINKVRQWPEPKDGEPRLHIKEQSEQEKK

SEQ ID NO: 273 - protein, 84 aa, Escherichia coli
MFSLIRRGQIYTDSSNWPVIIHSCSDHSVRIKRNDGELRTISIKRFNEDFERVEHDEYRKICAEIEQETNLKNLRAMRRGKITE

SEQ ID NO: 274 - protein, 189 aa, Escherichia coli
MNNLMIDLESMGKKPNAPIVSIGAVFFDPQSGELGQEFYTAVNLESAMEQGAVPDGDTILWWLRQSSEARSAICVDDAMPISSALSELSHFINRHSDNPKYLKVWGNGATFDNVILRGAYERAGQVCPWQFWNDHDVRTIVTLGRSVGFDPKRDMPFDGVAHNALADARHQAKYVSAIWQKLIPTTSNS

SEQ ID NO: 275 - protein, 78 aa, Escherichia coli
MSNIFQLAPNDWVCESVLIAVTGLKPGTILRARKECWMIGREYIHVSPDGNPKPSSECMYNRKAVDAWVASMKSKQPG

SEQ ID NO: 276 - protein, 437 aa, Escherichia coli
MDKVTYPTGVENHGGTLRIWFNFKGKRVRESLGVPDTAKNRKIAGELRTSVCFAIRTGTFDYATQFPDSPNLKAFGVSKKDITVKELEEKWLDLKRMEICANAFNRYESVARNMVPRIGGNRLVSAVTKEELLYLRKYLLTGYQNPTKNKAPAKGRSVVTVNYYMTTMAGMFQFAADHGYLEVNPFEGIKPLKKARAEPDPLSRDEFIRLIDACRHQQTKNLWSLAVYTGMRHGELVSLAWEDIDLKAGTITVRRNYTKLGEFTLPKTEASTDRVVHLIQPAISILKNQAEMTRLGRQYHIEVQLREYGRSVNHECTFVFNPHVVRRSKQVGFIYRVDSVGDSWEAALKRAGIRHRKAYQSRHTYACWSLSAGANPSFIASQMGHASAQMVFNVYGAWMADSSAEQIAMLNQKLADFAPLMPHSHENSTGGLLKSVS

SEQ ID NO: 277 - protein, 241 aa, Escherichia coli
MEGNTTLYALPKPEVVLRWREQTTDDFRFCFKFPATISHQAALRHCDDLVTEFLTRMSPLAPRIGQYWLQLPATFGPRELPALWHFLDSLPGEFNYGVEVRHPQFFAKGEEEQTLNRGLHQRGVNRVILDSRPVHAARPYSEAIRDAQRKKPKVPVHAVLTAKNPLIRFIGSDDMTQNRELFQVWLQKLAQWHQTTTPYLFLHTPDIAQAPELVHTLWEDLRKTLPEIGAVPAIPQQSSLF

SEQ ID NO: 278 - protein, 131 aa, Escherichia coli
MVSALYAVLSALLLMKFSFDVVRLRMQYRVAYGDGGFSELQSAIRIHGNAVEYIPIAIVLMLFMEMNGAETWMVHICGIVLLAGRLMHYYGFHHRLFRWRRSGMSATWCALLLMVLANLWYMPWELVFSLR

SEQ ID NO: 279 - protein, 247 aa, Escherichia coli
MSHRDTLFSAPIARLGDWTFDERVAEVFPDMIQRSVPGYSNIISMIGMLAERFVQPGTQVYDLGCSLGAATLSVRRNIHHDNCKIIAIDNSPAMIERCRRHIDAYKAPTPVDVIEGDIRDIAIENASMVVLNFTLQFLEPSERQALLDKIYQGLNPGGALVLSEKFSFEDAKVGELLFNMHHDFKRANGYSELEISQKRSMLENVMLTDSVETHKARLHKAGFEHSELWFQCFNFGSLVALKAEDAA

SEQ ID NO: 280 - protein, 323 aa, Escherichia coli
MIDFGNFYSLIAKNHLSHWLETLPAQIANWQREQQHGLFKQWSNAVEFLPEIKPYRLDLLHSVTAESEEPLSAGQIKRIETLMRNLMPWRKGPFSLYGVNIDTEWRSDWKWDRVMPHLSDLTGRTILDVGCGSGYHMWRMIGAGAHLAVGIDPTQLFLCQFEAVRKLLGNDQRAHLLPLGIEQLPALKAFDTVFSMGVLYHRRSPLEHLWQLKDQLVNEGELVLETLVIDGDENTVLVPGDRYAQMRNVYFIPSALALKNWLKKCGFVDIRIADVSVTTTEEQRRTEWMVTESLADFLDPHDPGKTVEGYPAPKRAVLIARKP

SEQ ID NO: 281 - DNA, 49496 bp, synthetic construct
  misc_feature 1..49496: note = Synthetic polynucleotide
  source 1..49496: mol_type = other DNA
aggcctctcctcgcgagaggcattttttatttgatgggataaagatctttgcgcttatacggttggatttcgcccggtttgcgagttttcagcaattttaatatccaggtgtattgttctggtcgcggaccaacaaaaatctcgacttcttcattcatccgccgcgcaatcgtatgatcatccgcctctaacagatcatccatcggtgggcgcacctgaatcgtcagacgatgcgtcttgccatcataaatcggaaatagcggtacaacgcgcgcacggcacactttcatcaaacgaccaatcgcgggcaacgtcgctttataggtggcaaagaaatcaacaaattcgctgtgttctgggccatgatcctgatcgggtaaataatatccccagtaaccctgacgtaccgactggatgaatggtttaataccatcatttctcgcatgcagacgaccaccaaagcgacggcgcaccgtgttccagacataatcaaaaaccgggttgccctgattatggaacatcgctgccattttctgcccttgcgaggccatcagcatggcaggaatatcgacggcccaaccgtgcggcaccagaaaaatcactttctcgttattacgtcgtatctcttcgatgatctccagcccttgccagtcaacgcgcggctgaattttctccggcccgcgtattgccaactcagccatcattaccatcgcttgcggcgcggtggcaaacatctcatctacaatcgcttcgcgttcagcttcactacgttctggaaagcagagcgacagattgattaacgcacgacggcgtgagctttttcccagtcgtccggcaaaacgtcccagccgtgccagaatgggatcacggaactttggcggcgttaaagcgatacccgccatcgctgctacgcccagccatgctccccagtagcgcgggtggcgaaaggatttatcaaactcaggaatgtattcgctattattttttttcgtttccatgcttttccagtttcggataaggcaaaaatcaatctggtgatagtgtagcggcgcaacttgccccgcaccaaataaaaaagccggtactgactgcgtaccggctgcgaatggatgttaattaatcaaaccgtagctgcggcacaatctctttggcctgtgccaggaattcgcgacgatcggagccggtcagcccttcggtacgcggcagttttgccgtcagcgggtttacggcctgctggtttatccatacttcatagtgcagatgcggcccggttgaacgtccggtattaccggaaagcgcgatacggtcgccacgtttcaccttctgtcccggtttcaccaggatcttgcgcaagtgcatataacgcgtggtgtagctgcgaccatgacgaatagccacataataacctgctgcgccactacgtttggcaaccaccacttcaccgtcacccactgaaagcactggcgtaccttgtggcatggcaaaatcaacacctctgtgtggcgcaacgcgaccggtcaccggattagtacgacgcgggttaaagttagatgagatacggaactgtttcgccgtcgggaatcgcaagaatcctttcgccagaccagtaccgttacgatcgtagaatttgccatcttcagcgcggattgcgtaataatctttaccttctgaacgcaaacgtacgcccagcagctggctttgctcacgtttaccatcaagcatttctcgtgacattaacaccgcaaattcatcgccttttttcagtttgcggaaatccatttgccactgcatggctttaatcactgcgctcacttcggcgctggttaaaccggcgtttctggcgctggcaacaaagcttcccccgacggtacctttcagcagattgttgacccactctccttgctgcatttcgctggtcattttaaaaccgttagcggcagtacggtcataggttcgggtttcacgacgagacacttcccaggtgaggcgctgcagttcgccgtccgcggttaatgtccaggagagttgttgaccgattttcaggttacgcaattctttgtcggcagcagccagttgggtgatatcacccatatcaataccatactgattgagaatgctgcttagcgtatcgccagtggaaacaacatattcatgcacgcccgcttcaccggcgattttgtcatccagttcgtcctggggaatggcttcatcttcttgtgcagcttgatcaatcggctcactggcttcaggtaagagcgaacgaatttcgttctgttccagctcaatggttttgacaattggcgtggcatcgcggtgataaacatagggccgccagacagcgacggccagagtaagaacggtgagcgaccccaacataacgcggtgtggtcgcggtaaattattaaacgccagggcgacagagcgggctatctgttgcacgtaatcacttcctcattaatctcctttcaggcagctcgcatactggttggctaattgattcaggaattctgaatagcttgttttacccagtttgatattcgtccccaggggatccaacgttcccatacgaacggatgtccctcgtgcgacgctctcaacgaccgctggcctgaactgtggctcagcaaaaacgcaggttgctttttgctcaaccaactgtgttcttatttcatgtaaacgctgcgcgccaggttgaatctcagggttaacggtaaaatgaccaagcggtgtcagtccgaactgtttttcgaaatagccgtaagcatcgtgaaaaacgaaataacctttccccttgagcggcgcgagctcgttaccaacctgcttttcggttgaggctaattgtgcctcaaaatccttcaggttggcgtcaagtttggctcgactttgcggcataagttccactaattttccatggattgcaaccgctgtagcccgcgctatctctggggaaagccaaagatgcatgttgaaatcgccgtgatggtgatcttcgtcacttttttccgcgtggtcgtgatcatcatcatcgccgtgaatacttttcatcagcagcggtttcacattctctagctgcgcaatcgttacctgtttcgcttcaggtaatttacttaccggtttttgcatgaacgcttccatctccgggccaacccaaacgactaagtccgcgttctgtaagcgttttacatctgatggacgcagtgaataatcatgttctgaagccccgtcaggtagtaaaacctccgtttctgttaccccatcagcaatggcagaagcgatgaacccaacgggtttaagcgaagcgacaacggcagcatctgcggcctgtgttgcaccgccccagagagcggcggataatgctgcgaaaagaagcgtttttttatgtaacataatgcgaccaatcatcgtaatgaatatgagaagtgtgatattataacatttcatgactactgcaagactaaaattaacatgacaagtctggtttccctggaaaatgtctcggtttcttttggccaacgccgcgtcctctctgatgtgtcgctggaacttaaacctggaaaaattttgactttacttgggccaaacggcgcaggtaagtcgacactggtacgggtagtgctcgggctggtaacacccgatgaaggggttatcaagcgcaacggaaaactgcgcatcggctatgtaccgcagaagctgtatctcgacaccacgttgccactgaccgtaaaccgttttttacgcttacgccctggcacacataaagaagatattttgcctgcactgaaacgtgtccaggccgggcatctgattaacgcaccgatgcaaaagctctcgggtggcgaaacgcagcgtgtactgttagcgcgagcattgttaaatcgaccgcaattattagtgctggatgaacccactcagggcgtggatgtgaatggtcaggtggcgttatatgaccttattgaccaactgcgtcgcgaactggattgtggcgttttaatggtatctcacgatctgcatctggtaatggcaaaaaccgatgaagtgctttgcctgaatcaccacatttgttgttccggcacaccggaagttgtttccctgcatccggagtttatttctatgtttggtcctcgtggtgctgaacaactgggtatctatcgccatcatcataatcatcgtcacgatttacagggacgaattgttttgcgtcggggaaatgatcgctcatgattgaattattatttcccggttggttagccgggatcatgctcgcctgtgccgcgggtccgctgggttcgtttgtagtctggcgtcgtatgtcttatttcggtgatacgctggctcatgcctcattacttggcgtcgcgtttggtttgttgctggacgtgaatccattctatgcggtgattgccgttacgctgctgctggcgggcggtctggtatggctggagaagcgtccacagctggcgatcgacacgttattagggattatggcgcacagtgccctgtcgctgggcctggtggtcgttagtctgatgtctaatattcgtgttgatttgatggcttacctgttcggtgatttactggcagtgacgccagaagatctcatctctattgcgattggcgtggtcatcgtggtggctattttgttctggcaatggcgcaatttgctgtcgatgacgattagcccggatctggcgtttgttgatggtgtgaaattacagcgcgtgaaattgttgttgatgctggtgacggcattgacgattggtgtagcgatgaaattcgtcggcgcgttgattattacttcactgctgattattcctgctgctactgcacgtcgctttgcccgcacgccggaacagatggctggtgtcgctgttttggtggggatggtggcagtgactggcggtttaaccttttccgcattttacgatacacctgcaggcccgtcggtggtgctatgcgcggcactgttatttattatcagtatgatgaaaaagcaggccagctaatctgtcgctgaacacatttgtcggatgcggcgcgagcgccttatcccacctgcggttcgctatctctggtaggcctgataagacgcgaacagcgtcgcatcaggcacactgccagtgtcggatgcggctcgagcgaccaatccgacttacggcatttctggcggcgtgatgccgaagtggttccacgcccgcactgtcgccatacgcccgcgcggtgtacgctgcaaaaagccttgctgaatcaaataaggttccagtacatcctcaatggtttcacgttcttcgccaatggctgccgccaggttatccagacctaccggcccaccaaagaacttatcgattaccgccagcaacaatttgcggtccatataatcgaaaccttcagcatcgacattcaacatatccagcgcctgagcagcgatatctgccgagatggtgccatcgtgcttcacttcagcgaaatcacgcactcgacgcagcagacggttggcaatacgtggcgtaccgcgcgcacgacgagcaacttccagcgcgccgtcatcactcatctcaagccccataaagcgtgcgctgcgactgacgatatattgcagatccggcacctgataaaactccagacgttgcacaataccaaaacgatcgcgcaacggtgatgtcagcgaacctgcgcgcgtggttgcaccaatcagggtaaacggcggcaaatcaattttaatggagcgtgccgccggaccttcaccaatcatgatatccagttggtaatcttccattgccggatacaacacctcttccaccactggtgaaagacggtggatctcatcaataaacagtacatcgtgtggttcaaggttagtgagcattgctgccagatcgcccgccttttccagcaccggaccagaagtcgtgcgtaaattaacgcccatttcattggcgacaatattggcaagcgtagttttacccaaccccggaggaccaaaaatcaatagatgatcgagggcatcgccgcgcagtttcgctgctttgatgaaaatctccatctgcgaacgaacctgcggctgaccaacatactcttccagtaatttagggcgaatggcgcgatctgccacatcttccggcaaagtggtaccggcagaaatcagacggtctgcttcaatcatcctttacctcataacgcggcgcgtagggcttcgcgaattaatgtttcactgctggcgtcagggcgagcgattttgctcaccatgcggcttgcttcttgtggtttatagcccagtgccaccagcgcagcaaccgcttcctgttcagcatcgtcggtcgccgggctggcaggagacgtgagtaccaggtcggcggctggcgtaaagagatcgccatgcaaacctttaaatcggtctttcatttcgacaatcaagcgttcggcggtttttttgccaatacccggcagtttcaccagtgcccccacttcttcacgctcaacggcattaacgaactgctgcgctgacattccggagaggatcgccagcgccaacttcgggccgacgccgttggttttgatcaactctttgaacaacgtgcgctcttgtttattgttaaaaccgtacagcagttgcgcgtcttcacgcaccacaaagtgggtgaaaacgatcgcttcctgacccgcttcagggagttcataaaaacaggtcatcggcatatgcacttcatagcctacgccgcccacttcaattaacaccagcgggggttgtttttcaatgatgatgcctctgagtctgcctatcacatgacgctcctgcgtaatgaatcaaagataatgctgtatgataaaaaaatgctggatagatatccagcgaaggatgaagaaaacttgcgaggtgtctcgatgatctgaaaaatggcgcagtataatttattctacagattatattggaagcaaatatttaaatattacatattcagcgaagaaatgtgtaataaaaatacacattgcgacccctgaaaaaaataaattttttatgctattacgtatattcatatctatttcaatggaatgacaacgtgaatattaattatcctgctgaatatgaaattggtgatatcgtctttacatgtataagtgctgccttatttggtcaaatatcagctgcatcaaattgctggagtaatcacgtcgggatcattatcggtcataacggtgaagactttctggttgcagaaagccgtgttcccctctcaaccatcactacgctatcccgttttattaaacgctctgctaatcaacgctatgctataaagcgattagacgccggactaacagaacaacaaaatcaacgaattgttgaacaggttccttcccggctacgcaaaatttaccacaccggttttaaatacgaatcttcgcgccagttctgttcaaaatttgtttttgatatttataaagaggcgctatgtattccggtgggtgaaatagagacgtttggagaattgttaaatagcaatccaaatgcaaaactcactttctggaaattctggttcttaggttctattccgtgggagcgtaaaaccgtcacgccagccagtttgtggcatcatccgggtttggtgttgattcacgcggtgggagttgaaacgcctcagcctgaactgaccgaggcggtataacttaacgcagtcgccctctcgccaggttcagtcgcgattcgctcatttgcatcgcattctgactaacgtggcagtgggtgatggcaatcgccagcgcatcggcggcatccgcctgtggattagcgggcagtttcagcaaggtgcggaccatatgctgcacctggcttttttcggcactaccaatacctaccactgtttgctttacctgacgtgccgcatattcaaataccggcaattcctgattcaccgccgccacaatcgccacgccgcgcgcctgccccagtttcagggctgagtcagcgttcttcgccataaagacctgttcaatggcgaaataatcaggctggaattgggtgatgatttccgtcacgcccgcatagatgagcttcagacgagacggtaaatcatccactttggtgcgtatgcatccgctacccaggtaggacagttgcctgcctacctggcggatgacgccatagccggtcacgcgcgaacccgggtcaatgccgagaataatagccatcacgcgtctccgttttgctgtttagcaggcctcatcagagagtcgctgcaacctcatcagagatttcaccgttatggtaaacttcctgcacgtcgtcgcaatcttccagcatatcgatcagacgcatcagtttcggtgcggtttctgcatccatatcagctttggtggacgggatcatggaaacttccgcgctgtctgctttcagacctgccgcttccagagcgtcgcgtactttgcccatttcttcccatgcagtgtagacatcaatcgcgccgtcatcataggtcacaacgtcttcagcaccggcttccagggctgcttccatgatggtgtcttcatcgcctttctcgaaggagatcacgccttttttgctgaacaaataagctacggaaccatcagtaccgaggttaccgccacatttgctgaatgcatgacgcacttcagcaacggtacggttgcggttgtcagacagacattcaatcatgattgccgtgccgccaggaccgtaaccttcgtagatgatggtttccatgtttgcatcatcatcaccgcccacacctcgtgcaattgcgcggttcagagtgtcacgggtcatgttgttagacagtgctttatcaattgctgcacgcaaacgcgggttagcgtccggatcaccaccgcccagcttagccgcggttaccagctcacgaatgattttagtgaagattttaccgcgcttagcatcctgcgcagctttacgatgtctggtgttggcccatttactatgacctgccataaaaatatctccagatagccctgcctgttcaggcagcgttaattacaaactgttcaatcgcctgccggttgctccaggacttagtgagcgccgccgcagcagacgcatcaagccacttgtaagccagatgttcagtgaaaacgatctggcgctcgtgcggaagcgcaagacagaaccatgattccgtattacgcgtcacgcccggcgcatagcgatgacgtaaatgtgaaaaaatttcaaactctaccgtgcgctgacagtcaattaaggtcagttgttcagcgacaacatcaatggtgacctcttcctttacttcgcgcatggcagcttgcggcgcggtttcaccctcttccacgctgccggttaccgactgccagaaatcgggatcgtcacgccgctgcaacatcagcacccgtttcgtatcttgtgcgtagatgaccactaagatcgaaacgggacgcttataagccatatcagttattctcagccttcttcacaacctgaatgctcagctcagccagtgcagtcgggttagcaaagctcggcgcttcagtcatcaaacacgctgccgccgtggttttcgggaaggcgataacgtcacggatattgtcggtgccggtcagcagcatcgtcagacggtcaagaccgaatgccaaacctgcgtgcggcggagtaccgtatttcagggcgtcgagcaggaagccgaatttctcgcgctgttcctcttcgttgatacccagaataccaaacaccgtctgctgcatatcaccattatggatacgcacagaaccaccgcccacttcgtaaccattgatgaccatatcgtaagcgttagccaccgcattttccggtgcagctttcagttctgctgccgtcatgtctttcggtgaggtgaacggatggtgcattgctgtcaggccgccttcaccgtcgtcttcaaacatcgggaagtcgataacccacagcggtgcccatttgctttcgtcggtcagaccaaggtctttacccactttcaggcgcagtgcgcccatcgcgtcggcaacaattttcttgttgtcggcaccgaagaaaatcatatcgccatcttgcgcgccagtacgctccaggatggcttcgatgatttctgcattaaggaacttcgctaccgggctattgataccttccagacctttcgcgcgttcgttaactttgatgtaagccagacctttcgcgccgtagattttaacgaagttaccgtattcgtcgatctgcttacgggtcaacgatgcgccgcccggaacacgcagagcggcaacacggcctttcggatcgttcgccggacctgcaaatactgcaaactcaacagatttcagcagatcggcaacgtcggtcagttccatcgggttacgcagatccggtttatcagaaccataacggcgttctgcttctgcaaaggtcattaccgggaaatcgcccagatccacgcccttcacttccagccacagatgacgcaccagcgcttccatcacttcacgcacttgcggcgcggtcatgaaagaagtttccacatcgatctgagtaaattcaggctgacggtcagcacgcaggtcttcgtcacggaagcatttaacgatctgatagtagcggtcaaagccggacatcatcagtagctgtttgaacaactgcggggattgcggcagcgcgtagaatttacctttgtgcacacgagaaggcaccaggtagtcacgcgcgccttcaggcgtggctttggtcagcatcggagtttcgatgtcgaggaagccgtggtcatccataaaacggcgcaccaggctggtgattttagcgcgggttttcaggcgctgagccatttccgggcgacgcaggtcgaggtagcggtatttcagacgcgcttcttcggtgttgacgtggttagagtcaagcggcagaacatctgcacggttgatgatagtcagcgaggacgccagtacttcgatttcgccagtcgccatatcgcggttaatatttttttcgtcacgcgcacgtacggtgcccgtgacctgaatgcagaactcattacgcagttcagaggccagctttaacgcgtccgcacgatccggatcgaaaaatacctgcacgataccttcgcggtcgcgcatatcgatgaagatcaggctaccaagatcacgacgacggttgacccaaccacacagagtcacctgctgccccacgtgggacaaacggagctgtccacaatattctgtacgcatgagatatcccttaacttagctgccggcggatgccccctgctgcgcaggtgaccaagtcgcagcgttagctgtatgtcacaactgaatgaaaaaaggcggctattatactggaaattctgccgcaccgtaagagcctggcccgcgctggaacgcctcgttaccactttatatcgggcctgaaatcagactctacgccagtttgctataaaggtgttgcccgaactcataaaaattaacaaaatttgtcgttccgccatcggctaatcgcattaaggtgagaggcacgattttgttttgtcaggagtcatcatgcttgaacttaatgctaaaaccaccgcgctggtggtgattgatttacaagaaggcatcttgccttttgccggaggtccacatactgccgatgaggtggttaatcgcgccgggaagctggcggcgaaatttcgcgccagcggtcagcccgtgtttctggtgcgcgttggctggtctgccgattacgccgaagcattaaaacagccggttgatgccccctcccccgcaaaagtgttgcccgaaaactggtggcaacatcctgctgcattaggtgcaaccgacagcgatatcgaaatcatcaaacgtcaatggggtgcgttttacggtacggatctggagttgcaattacgccgccggggtatcgatacaatagtgttatgtgggatctcgaccaatatcggtgttgaatccaccgcccgcaatgcctgggaactcggttttaatctggtgattgccgaagatgcctgtagcgccgctagcgccgagcagcacaataacagcattaatcatatctacccgcgcatcgcccgtgtgcgtagcgttgaagagatcctcaacgcgttatgatttacatcggtttgccacaatggtcgcatcctaaatgggtgcggttggggatcaccagccttgaagagtatgcccgccactttaactgcgtgacgcgggcattttaaaaatcactaaagaacgcccaagagcatgtgttttctttagtttattcaatgcattaaaaaatagtttcgcatgaaattcggtaaacttcatgtgtgcaataatgtcccattcatgccccaaaatgccccaaagcagacatttttgccccaagtatgccccacaagtcacgtcttcaagtcgtctatatccatagcacaccgagttacattcttgcatccggggtgtcgacaatacctactttattgagtgtgcgagaattaccaggaacctttccacaatgtagtagtctaatagtcgaatccatctaacattaagaagcgttatgatcactagcctctcattgatatcttctgtaatagtcactctatgtatcatggtgttcgctacagtaaaggtagggattggtttgtctaacaatccagacagaaatgataattaacctcaaccacgtaaccacacttcatacttcatacttcacttaacagtgaagtgctcacatcaccgggcagtcatcaaactccgcattcctggcatcattaatgatgtacgtgatcactccaaatatagcgggtgcagaactgtaaccatcatcatctgctggcagcgcttcccttctcccgttatccagattaaccaggtgcggctgaggatgagtccgatatcgcttgatcctgaattccccgtcgattgcacatatcagcagtgaaccatcgcaggcagtaagtgacgcatccacaacaagcaacgctccctggattatcccttccctgaaatgtgaacgcgatgcccgcatgaaataagtcgctgcgggctgactgattagctgctgatcgagggagattcgtgtttcaacataatctgccgcaggtgaaggaaatcccatgtttacgccctctcttgaataccggataaaaacacagtataaatactgtatatccatccagcaaagaggcaatgagcaatgttcgtggaactcgtttatgacaaaaggaattttgatggtctgcccggtgcaaaagatatcattctgggcgagttaactaagagagttcaccggatcttccccgatgctgatgttcgggttaaaccgatgatgacactgccggcgatcaacactgacgccagcaagcatgagaaggaacagataagccgtactgttcaggaaatgtttgaagaggctgaattctggttagtgagtgagtaaagattttcaatgcccgccacagttacgtattgattatgctgtggaggatattcattttcgtaaacgttggtttgggagaagcggcaaaacggaatgtgggaacaggggaaaatcagataccagatatgtctgcatttccatctggcaataactggtttcagttaccaagtggacatatcgttcagatattttccatgaacgttcttggtgcagatgctaatggcacgtcagctaattaccccattgcttttccaacaacgatgattgctgtcagtgctctatggtctgatgggactgtagcaaatgcaccgacatacaagatgatggggaacacgactaacagaacaactttgacgataaaagtatcagccagctcaggtacttacgggacaatgattattgcggtgggacgataatatgaataaatacagttactctccttcagaaaatgccttttatgctgttgcgttaaaaaatacctatgaattgagtggcacatggccagctgatgcattagatattcctgatgacatttctgtaaaatatatggcggaaccgccacaagggaaaatccgagttgcaggggaaaatggttttcccacatgggctgaaatacctccaccatcacatgaggaacttattgaacaggccgaatcagagaggcaattattgattaaccaggccaacgaatacatgaacagtaaacaatggcccggtaaagccgctattggtcgtctgaaaggcgaggaactggcacaatataattcgtggctggattatctggacgcactggaactggtcgatacttccggtacgcccgatattgaatggcctacgcctccggcagttcaggccagatgacatccggcgcggtgctggtatctgttgcagtcaccgcgtcaatgtaatccagcacggcgttaagtcgggttgtttctgcctgagtcagtttccgtccggcctgtaatttcagctgaatcagactaatggaagccattgctgcatcaatcagtgattggcgctgtgcttctgccgcttctactgaggcaccgtgttgtgcctcagtatctgtcacccatttctcaccatcccatttatcatatggcgttaacggtgaaagcgtgacataaccgtttttgatggcaccgatataatccactgtaacagctgcgccattttcgattgagtaaacagtctcattgcgatggtcttcctcatggctccatcccttacctgtaaatactgccactcttcccggaatgttttcgtccgggtcaataccagtggaacaggcgggcatacttacgccagtattaatatattcatcagaccagcccgtatattcagacgttactgcatcataataaaaacaacgcatatcacccggcactgcagccagcccattttcatcaaaaacaggtttcattatttagccctcaccagaaagttaaatgcaatatttcgcggtctgacagcaacaaaattcacaccatcacccacagagttactgttgaaattaaatcgtgaaaatcctggctgatttccggcgatgccatcatgaaagttaattgcgtgtccagcacctccgcctatattcccggcaaactgagaaaagtttgtagcttcctgccagcttaataattcgcgaccaccatctgcacctcgcccgtcatcccagacacgaatgaaatcaccgcgggcttcaggtaataccagcgaaggaaacactttcgccagcacaggataatcagtggcagagaatttcgcgccgttgaacttcaaaaacaccatactggaccagctgtcgattacagtatttggcattgcagcggacggccagaagaacggaacgccaatagctggagaaccttctcccaaaccaaggtttgtgcgagcgtctgcggcattcgttgcgccggttccgccgtctgcgacagtaaccgcaccgttgctccctttctgcgcaagtttaccgatgcctgggatggttacggcggtgccgttgatggtaactgtgatgctttggtttgctgaggtggtggcgaacgtctcccacgcgccaatattctcgtcgtactctttgatgagctgtgacatggcctgcgccaggccgtcgactgagatattgtccgacacaaggattccatacttctggccgctcagcgccggggaaactgctggcgtaaccgtcattgacgtggcgctgttcacggatgaaatctgaaacagctgcaccgggttagacatcacgataatcgtctggccagcgcggacctggctggcgggagctgtccagtttgtgccggagccggttgcggtatttccgttaatagagatagttccggtgctataaatcataacaactcctaaatttagacaacatgaagcccggagaggtatataaccctcaccagaaataatttctgaattggtttttaatacatgttgggcaacgccagtgttggcatagctatagttgtatcaaatgccattgaccacccacccaaataataattgccgactactttattcctttctgctctgacattcccacctgtcattacaattcctttatacctaatatttccgtaaccaccaacgtgtcgacagttagccccggtataaacaatctggcaaaattcatcaccaatattcaggttcgcattggcgatatttattgtcccatcaaaaatgaatggcttcgtgcaagtaagaagacgtttgcgttgacgattggtttattcaatccatcaactaaaatatcatgtaaacgaattgccataacatttcccactttattttacttactcaacacaacaagtatattaaaattgaaccttgtcaacaacacaaaggagtcccaatgaaactcgctctaattatgctgccattatgtctgtccctcactgcatgtggtaatggtttaaataccggtaaaccaaattccggtgtcattccaaaacctttggatcgagatggtaacggttctttaatttatgataccgaaaaccttccaatgacggggcagtggtgtcacgagattgatcacgaataccgacgaatcggtagcccttctaactgtgttatagactactaaatattaacccctcaaaagaggggttaatattttaacctgtgaatgaaccagatccgtgtgatactatgatagtaggcgaagaaattgacgcacccctgttattttgagcggacactttaatacgcactgttacgtttggacctgttacaaccgcagaatgcatgataagtctctccatgtcttgcacggatgaaccactctcattgccgttaatgtcgattgttcctgcaccattacctttaacgtttgctataacacagacgtgtcttgcgtgcccagaactggatgaatcattataagtcattaccttttctaaatatccgtcactagaccgactcacattcgtccctgtgtgcatatttgcaatgtccccgataaaactttgggcttccacagtccctttgaatttaccgcttgttgcttggatctcaccagtaaagctaccgccactagcatatactacacctctgacggtcacattgttgaattcagcatctccagctttattcaacttccaaccagcagaaccagctgcatagttgttggactggatatagttaccgatttttgcgttctcaatggtgccgtcctggatgaagctggcccggatgaatgtctgcccgttctggatcacgaacggcaaagctacgctatttccggctgccgtggtgacggcgaagcggtcagccaggaagataacctgcgactgcatgccggatggcgtattctccacgccgatacccatccccgcggcgtaatactgcccgttgctggagacaccaaccttgatgttgtacatcgcgctgagttcgccattaacgttggctatagcctgagcgttagtggtgatggcggaggtatgcccgttcacggtcgccgtgatgccgtttatctgcgtggcggtggcctgctgatagtcggagagcgtctgattcaggctgttgatggatgccttgttgccgttgacgtccgtctgcaggctcagcaatgaacgtgctgtggcttccttctcactgacgatcacctcgtcgagacggtccagattcgcgctgttgccggcgaccgatgcagaaagggttttacgcgtggccacctgagcgaggttggcctggattatcgcaattgcagagttcttcaccccgcccgtcatgccgtccatagaaacgctgatgttgtcgattcgctggcccagggcggtatcagccgtcgcaacggtctgctcaagctgactgagtgaagacgaaacattcccgaccgtgctggaaagctcattaacgctggtctgaaccttcccgacgtcctgggcatttttggcgatatctttcgcttgctgctccagttcgtcgttggcctgtttgatatcgttagccatgccagcaattttttcgttgctgtccaccgcgttctcgatcaggtctttgaacgtttccgactctttcatatcctccagaatgtcattagttatttcgctgacatctatcgaggacgtgcccatgatccagtcggtccagtccccggcgttaccgatacggtcaatcaggcgcgcgcggtaccactggcgaacgccggcaggcatggggccatgctgataatctgcagccgggtacggcaccaggaccagcagttcaggattggcgtagtcggcagttgtggcgcgctgaatctctgtataggccgtgtcgcctgagccatccggaaatttccaggtcaggtcgatatgccagaccacatcttcggtcgccaggaagttgagcggagtacccggttttcccgttttaccggagagataagttgtttcaccgtatccccatggtgacgacgtatcctgcgcattcagcgcccgtacgcgcacgtcatagctgcccgaataaatgccctgaaccgagaaaccctgcgcgctggtaaccggaacgtttatccagtccccgttgtccttacgccactgggcaacataccggattgcgccctctaccttatcccatgacacgtccaggcttgctacagtcagcccctgagacacatgatcgctctcagtcaccacgatattcttcggagcagacaggacgcttatcggcgtgacggtgatcgggggagactcgacccgaacgccgtcatcgatgtaacgatatttgtttggatcgtgctgaacggccgtaatagtgaaaccgcctgtactgtcgtcgttagccgcgattgaggtgaccctgaagtactgtattgcgaggttatcactgtctatcgcccaaacagcgcccgccacaggaacctgactgaatgccgtagccaccgtcaccgtttttttatcggcgctcaccgcgctgattgtccgcgtctgggcttttccgtcgggaaggttaaccaccagccggtctttcgccgcgtagtctatttctcgatcgagggtaatttggcggccgttggccgcgcttatacggcccccgttctccttaccagagcggaaaggatcggcgacaccgataatttcagcgggcaaagggatataaccgtccagccccacgccaaacgatacggtcccgtctttggcattggagagcaatacccagcgaccgcgtcggtgcgcttcactttgcgaggtgcagccgattgcggtcagggacgtctgccggacgtcgtaacgttctacaagcgccgaatcgtaaaccccctcaacggtatcgctgtaatggttctgcggatcggaccaggacaccaggcaggagctgtagcgattcttgtatgagccgcccgcataagtaaacagcccatcgataacgtttgagacgttataaacccagtcaacatcgtcctgcgggacgtctgcctggacataaatctgatcgtttccccagaacgttattccacgaaataccgcggcgagatcgttaagtacctgccaggcgtcctcctggctctgaatgaaaacgttgcaggtgaaacgcggttcggtgccaccggccccgtcggaaaccatttcgtcacagtactgggcgattgaatacagcgcccacttatccaccatggacgcatccacgcgcgtgcccatgccgtaaatttcatccagaaccagatcgtaaaagatccaggcagggttattggaccatgccattttgaacccgccggaccatgaaccagaataggttcgggttttcggatcgtaattatccggaaccttaatcagcttgccttttatcttacaggtcactttcggcgcgctgccgttgaattggctgctgtccacttcgacatacaggagcgctgttaaaggataacgaagcttgctgtcgatgacttccgcatacgaaaacaccttgaaggcgttaaccagtttcgaatttgatccgctggcatcagccgtaatacgcctgaccctgacagaccagccggacgtggattttggcagatcgatacggtggtcacgctgatattccgtcgtggtctttccgtcaaacttgccgtttacaaccgttttccaggcgccgccgtccgttgataaatcgatcgcatactcggtgaccgtgcccaccatatcgccattatctttatagagatactggaccggaaggctgagcttgatacggatggcatccagggaaaggtttgtaaactggcgcgtccagggcgcggtggtggtgacagttgtgcccacggccagctcgttgtcgacctggggcatcccggcaatataggtctggtcctgtgtgcccttgcggaactcccatttcacgccgctgaagttgtattccccgctgtcgtttgccagcggcgtatcgttgagaaaaatgttctgagcggtcaggtcgccctgtatttccccctcagaaacggcaatgagcatttttaattttgcgaccgacagcagatcgtcaggctgctcaaccggagtatgtgaactgccacctccccctttggcaccctgcaggatggtttcttgtttaagaagctgcattttttcacccataaaaaaaggtgccgaagcacctttaagttagtggccgctggcctactgctgatcgctcgagtacataccggcgctgactatcgctccccctgcctcagtcagaccgtaggccagggggacaggatgccccatagcgacggtattgaccggcgccccgaaggcgtagttaggcgtgttgtccgtgctggaggatttacccgcgccgaaggatggctggggcgtgagcatctggacaacgccccccagcatcatcgacactccgacccctgtcagaattgacgtggcgctgatagctgttgcactcatcgccgcgccccaggctgccatgctcgcaccagcggtaaagaatgcagcgaccagcgcaacagccccgacaactatctgcaggacgcccgaacttttggccccctcataaacgggcacgatccggtacacgcttccaccgcgggtcatatcaaactcttccagcccgatattgttgccaccgttaaaaaaggcgaaacggatccccttcatatgagcttcagacatatattttttgaatccgggaacctgtgaacacatggccctgagcatctcgcgcaggtcggcaacatcaaactgaacgcgtttaccgaatttttttgccattttaccttcgagaataagcgtcttaaccatgcattctgtccttatgcctgaccacccggaccgttctgtcgcgataatattttccataaggcgttcgcgaagaaaggtgcccgaaaagatgatggagaatgatgttatcacccacatataccgcggcgtgattagtcaccgatgcctgcacactcatcatgatgatatctccgggctgcattgcaccggcggcaatctcaacgaatccctcacgctcccagttgtcgtcgtagagacgctccttgccgctctcccaccattcgtaaggtactgaataattgccgagaacaatgccgtattcgcgcagataaaattcacggataagcgaccagcagtcggcgtaacccagcacccactgccgcccggcataatcccggtcttcacgcggggaaatcgtacaaaaatccccgtccggccaggacatgatcccccactcaatccccgaccagtcgcactggatccggtccagctctgagggcaccagccgaaccacatccggatgggaatgaatgagcatgatgatctcaccgcgcgcgcgggcagcgagctggtcttccggggagagcgtgaatgtctcctcgggtttatcggcaatgttgcggcagggaataaagatttgttgctggcctgactgaacaatcaggccgcaggcttctttggggtattcagcagcgacgtgctgacggatagcatccagcaatttttcacgcatttttatttcccctgcaggtttgcagccggaaaaccgccgaacggcagcggcgcatccgggccgtgacgatcctgacaatcctgccggcggccgccacaaacatctttcgacgggtcatcggtcggtgtaccgtctttggtaaagtatttcgtgccgttgtaatcgcatccggtcccgcttcggtaccagccccgcatacaccaggtgcagacaggcgtaatctgccgtgtcggcagctgcaggctctgaatatcgaaaggagaacacagctcgaaatcaacctgtacccgcgtctctgcggttttagcattgacgtaaaagagctgtaagcgctcatcggccgggctggcacccggattaccgtttttccagttggcggcatcgagatacttcgaaagcgtggtatggattttgaccttagccctgaccatatcgtcatattcaagacacagcgcggtgacatagtttccgacgttcccgacggacagcgtgggcgttggctgggaacctgtactcgataactccatccccttaagttcgtagggatggggatcgtactggtttccctgccagataatggcgggcagattttctgcggcgaaggctgcccacccctcttcctgaatattgtgcgcatgaaaacgcagcacctgatccataccgaattcagtgccgtcgatctcaatcagctgaataacgctgccgggctcaagctgttgtatgtctgccgtaaaactcatactccccccataaaaaaaagccgcccggaggcagctttcagtgtttttcgagaaaatcagggcgcgaacgcctgttcaaaagtgaaggccacagtggcttttttcccggtagggaaagaaacgctgaacgaatcggccttcattctgaacagctttttttcaccccatggagtggtccaccagaacgatttagtaacgtgagacatcaggaaagcgcgcagcgcagccgcctcctgtctggtgcccgtccagtccaggttccacgtttcctgtttgtcgttgatccccatccccgctatctgtttgtagccatccccgaactgggcctgcagcgttcgggctgtttcagtgccctgcgctgtttttcgcgtgcgccaggtaaacgtgtccgtcactgtgtcctcctcgaataaagcacgccgcccgcggacatttcttttttcagtcgctcggtgattgtctgctgaacaatcgcctgcagctgtttcgccgtccccgtggcgttcgcctgatttatgcttccgtcactcccctgctggctgatgctgactggggcataaacactgatcccgcccatgccagcaccggctgcgttcccgccgccgaccagaccacccgaggcatacccgcgcatcaggcgatagagattagccacgccgatgcggctggttgattctttggtgaagacgaattccccgcggtgaacgataccggctggctcgtacttgccgccgtgcccggtaaaaccgcccacgtcaaaaccctgtggccggtatgacgggaccgcgaatgactgaccggcagaggaggttttcgccccgccgctaacccagcccattgcactctggatggtgtaagccaccagcagctggttgataacggacacaatcattttaaggatcgagctggtgaattccctgaagctcgccttcccggttgtcgtcaggctggtaagctggcccgccaacccgctgaacgtagcctgagaaatctgctgaacggagctgaaaacgtttgtcgctgaatcctgatattcggcccaaccctgtttcgcaccggccagccagtttgcacgcagggcatcttcagcttcgaacgtcgccctttgctcttccagaaccttttgctgcgcctgagggttgtacgaatagctttcgctgagacgctgcagcgtagtttgtcgcccggcttcccgggtggataacccctcagactgagcctgcaggcccgccctggcggctttttgctgctgctcaaacttcacggcctgatcggccagctggttgagcttttgctggctggcaaccttatcgcccaggtcggccagctgccgcttgtactcgagcgtttcttctttgtgcgccagcagggatttttcctgcgccgtaagctgacgacgcccagcggcctcctgcagaacggtgaactgattttcagtttgccagagatcctgacgctgtttacttatgacgtcgttcacgctggtatgctgctcaagcgttttaagctgggcctgaagggtgagaagttcggcctgcgccttttcctcggctttgtccccggcgggcgttgagtagcttttgcctttcggtgtttttggatccttccactgcttttcaatcccggcgcgggccgcggcaatgtccttttcagtccacagcgtggcgacaccgtctttcgcatcctggcggtttttctcaataagctgactgagctttttctctgctgaagcccgcttttctgccgccgtcgcgccggactccaccagctggttaaactgctgctggctgcggattgcctgagcctgctggtccgttcgcattttttcccgcgcggctgccagcccttcctgggcgtattgctgatcggcaagatcgtaagcctgctttttcagctccacctgctggcgcgcgtttctcagcctttccgcatccgctttctgcagaacgttgttaccggcataatccgggtcgaccttaagattgctggacagcgcgcggtactctttctctgctgcctgccactcagcaaaagagtcctggcgcttcatcgcggtgtcaggattacgcccgacgcccagcatcgcatcccacgcaccggaggcggcattcttcacccagttccaggctttttcgagggatccgagattatcctcgaccgcaccggcgcgctgaatgaccgcgtcggaatatgcccgcatggccagctcggcagccttctgagaatcccccagcgcctgagcagaagctatctgttcatactgggtggctgtcagaaaatgaagggaatcgttgagcgtcgcgaccgcgttaaccggatcatccttcaggcgtttaaactgatttatggtttcgtcaacggcctgcccggtagcctgctgcagcctggcggcaacattgctgaccatgctgacgtcattaccgctgaacgcgccgctgccaacgacctgcgccagcacgcctgcagcggcatgctgagtgatgccattacctgccagcgagcgcgccagcgcctgcagctgccctgacgttttccccgcgtagttcccggtcaggatcagctgcctgttaaattcctcagactctttgctgccgtcgtaccaggccttacccaacccgaataccgccgcggcaatccctccgaccatgctggcgatcccaagaccgcgcagtgacagaagctggtctatccaccctgcccggttagccagcgtgatcccggagccgcgcagcgcgccgaagttaccgcgcatgacctcgccgatcagtattcccagttcctgccgggcggcggcactttgcagccccagaccgtgcgtggcgactttggcagcttcgagcttgcggatatagacttcagccgcatcgctggcaccgacctgcgccgccttcatgcgcagtagctcggtaccggagagcttttgctctgcaacctgttgcttcagctggctgaggaatcgcgtgcgcgctgcggccgatttttcctccacgatctgcagttctttttgacgggccgtggtgcgggaaataagggcgagataatcctgctgggttatgttgccctgtgccctcgctgcgcgaaagcgcgcctgcacgttcgcaagcgactgtgtttcaccattgagctggcgtacgccgtcgatctggcggaaaaatgatgccgcaagttcatcctgtcgacgggcaagcgcagcggcctgcccgtcattctcacgcatgcgctgattaagctcggtcacgcggcggtgagtttcatcaacggactttgaaacgttctgccagtctttggtaagcccttccgttgcggccgactggcgggatttcatatctgcggcagccgccgcgccagcgtcacccacggttttaaacgcagccgcctgccgctctgaagcgcgctgcattcgcgtctggactttttcagagtcctcagccatcccggttagctggccctttatgcgggcaacctgctcactaaacgtggcgctgtcgacgtcaaggttgatgaccagatcgctaatctgctgggccatatcggatacctcctgttatcccctcagctgcggccatcagcgcatcatcatccggctcgtcatcgctgatgacgataccggaaggagaaagcaggctgaaatgtgcgggggtaagttccgggtcgcggaagaaaagagtggagatggaataaagcagctctgagaaatgcgcatcgagctgagcgtcctgaaaataatgctcccggtagaactggtgccagtcgcccagctcagtggaagtcattccagccagcatggcgcgccagtcgggtcgcccgaactcgcgcgccagattcaggacaaacttcagctcgctggcaagggcttttccgccgcaacgggttctgcgctttcggcctccgctggggcatccggatcggcagctttgtcatcctcaaccggaacgagcatgccggagagcagctttatttccatttctgctttaccgatcgcctccggcggccagccgctaagcacctgctggtaaagcgtctccacatccgtgccagccggatcgttatgccacaaagacatcgcaatcaaacgcgcaccgcagcgaatatttgagccaatcagcctggccgtcatttcctgatcgctgatgccgtcgctgtcagcgctgacggccttttcctctgcggccataaacgtgatgtactcaatacgctgaagcgccgacagctcgaagatggtcagtgattctttttgccaggtgaacttctcttttttcagaaacatgcgtccttccttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcataagtacctcttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgattacacgtcttgagcgattgtgtaggctggagctgcttcgaagttcctatactttctagagaataggaacttcggaataggaactaaggaggatattcatatggaccatggctaattcccatgtcagtccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcatgctcattcctcagttgtaaaaaagccccgcgagtgcgaggcgatttgattgaattctcggctcttatctcagcgcagccccttactgcgtgccggttgctcggtgatgagcatcagcgatgagacattaaagccgaccgaaggccagcggcgttcctcatgttgccgacagagccatatcgacaagaggacgaaaactagcagcatgaatcgcctattggttattcgacagtcgcactgattcgtaaatccgctcacacgtcattcctgcccggtagctttcgtcagatcgtccagcataatatcgagctgcttctgcaaggcttccgagcatgtcggcaagcattgctgcgttggctccggctgttttgcttctgacggaagtggcgagatctgcggtgtgctttgcggcgtccatgtgggtagcgagttttgttgcttcggcgcgcagctgcttaacagtggtagccaggccagcagaagtaacggcagcgctcgctgcttgagcttgagcatctttaacggcctcatcccgggcgattgttcgcccttgttcaatcatacgagctgcggtctgtgcattcgcttcctgtgaagattccgcgctatcccggtcagcccactttattttccagctgcggttcgtccactcactgccagcgagaaacgaacctaccaacgcaacaatcacaatgatgcagatgccacccggcttcactggtctatcccccagcacgtcagtgcgctttcctggtcccgccgttctacctgcccataacatccatccttctggcccttggtcaggcggcaatcgcggccaccgtctttaatccaccagcggatagcttcacaggctcctttcgtatcgccagcattaattcgcttatagaacgtagacgggaaacattttccggggccgatgttatatgggcagaaagaagcgatacccgctttctgtggttcggtcagtggtactttgatatttcggtcaacccacgccagcgccttgtcgcgttcaatggcgtttacctgggcgcatttctcagctgacagcttcatgccctgtactactggcttgccatcaaccattgttgcaccacggcaaatggtccagagtccgccgccgtcgcgatatgctgtcaagctgttaccctctttctcatccagaaactgatcgagaatcacgggtgcggaagccccggcaagaatcaaaccaacgaccgctgcgctcaatttattcttcagctttagagacatagccattgcgccgatcctcccgttctttccagcggaaataccagttcactgcacaggtgattaccgtgcatgcgataccgacaataattgcccagtcgctcaggcttaaccctgcaattctgtcggccaacatccaggacacctcttttgctgttttagctgtttcggcatatgccttcgctgatacaccgcagccggcaagcgtggttcctgatccatatgaaagtctgctgtaaatggtgctcattctggtcatagcctcacctccgatagttcggatggcgctgtgtgtgattgaaggggatcaggcaaccgggctcttatgttcaagtaaaaattaaggatgattcccggtgcctgaagatggtgatcaccacagcaacgggggagcgtggtgatcgttatgattttttcagtttttccacctcttcggtggtctgtataaacctgtctgcctccagttctacgccgatcgcccgacggccaagttctattgcagctttcacagttgaaccagagcccataaagaaatcggcaacgatatcccccggtctgctgctggcgctaatgatctgtttcagcatgtcggcaggtttttcgcatggatgtttgcctggataaaactgaacaggcttatgtgtccatacgtcggtataaggaacaagagcggaaacagagaagcagcgccgaaggattttgtattcctccagcaattctgaatacttgcggtttaatgactggtaggtagccaccagctggtggtgaggatgttcaagcttttgctgaatatgtttatcgatggcgatccgcgtgaacagttcctgcaattttcgatagtccacttcattcggtagttgccattggcttgcaccaaaccagtgtgacgccatgtttttctttccggttgcctcagctatttctttcgagctgacacccagtgattcacgggcattacggaagtaatcaatcagcggcgtcataatgtgctgctttagctctgtgcttttcctttcgtaaacatcctctttacctgtatacggtccaagatagtgctcagcaaacaaaatccgttccgtagatggaaagtacgcacgcaggctttctttattacatccattccagcggcccgatggttttgcccaaatgatgtgattcaaaacgttgaatcgggcgcgcatcataatctctatatctgaggccagtcggtgaccgcaaaacaggtagatgctgccagcaggtttaagaacgcgagcatactcagccaggcagctatcaagccagcgtaagtagtcctcgtcccccttccattggttgtcccagccgttgggcttcactttgaagtacggaggatccgtaactataagatcaatagagttatccgggagggtggcgacgtaatgcagactatcagcgttgattaactcaacactgtttatttttacagtatttttcatagatcagtaagcgtaactctgataggctcacgttgcttttgcgctaaagcagtgggccttggttagcttgtgacctgaaagcatgagctgatggctggccgggtgcgctaacacccaccagccgcccatttccacagcagaaaacccccattactggaggcgtttataacatccgaactggtaatcagataaccccgccatcaccagctgcgtaagtatgagctggcaacgttcgtggctgaggtgggtattctgtgcaatctccccagccgttgctggtttatcgcttaattcattgaaaacagcctttgccgtttctgtcatatcttcctgatttagcatgtcttttacctaaaattagttgcgtgacatacagataactctggttggtgataccagcaagagaagaatttgattctgcaaccaacaaggcctttaggcatcaggcaggaatgagatgcaataaaaaaaccacccgaaggtggtcttatatgaatctttaacgcggacttagcaaatattccacatcatcgtactaccgttatggttttcgataatttttgcggctgggctagtaccaaaagagtgcatatagcaatgatgaatagtaaggaccagatcctgcaacgtttggtcactctctagctccatgatatttaaaccaatattttgagctttgtccaaatgaatatgtctggcatgtgcatacgttgcttggtggttgtttaactcatcacatatacgcttagccttagcttcagcgtcagcctgacctgcgaacataccagtacaaagccatttctggacaatttcgttcgcccagagaattgctttttcacactcgccaatcaacgttggatttagtttttggaacgtaaattgccaccattgcagtgcagcagggttggcaaaaatttccgcttttgctctctcatactcctcaataattgcatgagatgataacccattaaactgtggatcaattggccccaagttcgactgtttacctaaaacgatctgctcagcacaacaagcaagcattgtgccacaactcattgaaatcataggtacaatcgctcggatattggttccgaactttgaacgaagataatgaccaattgattctagagctgcgatatcgcctccaggagtatggagtaagatatccaatcccagactcgtatctaacccattgatagcagacataagaccatttttatcatcatctgacatctggatcagatgttgaaacccaggcccccctttttgaaggaagcctgagtaataagaaattacatttcggccagtatgtttcgataaatcacgtaagtacttgtggcgaacctcatccgctggtgtacgttgagcgatagtacccatctcacccaatacgtctatccaatttggcatgttatcaatttatcagtatgagtacagttggtgagattgctgaccgttctgctcagtagtatttggtgttactgtgctgtatgaatagagcacaccacttctcacattcagatcgttttgctgagcgagaacacgcatagcaaaatgctgtacggattcgcctttttgaaactcttggggttgtatgcccattttttcgtaaaattcagcagcgctcatgatatgtccctcgtttttttctacatctatgcaattccaggagccatcaacacaagatgtagtagttagcagtcgtcaaatacacgaaaagcctcaagatgaggcttaaaaagattctttttgataaagatttagccaaactatagcggtcaaaatgcagatttgacaagtataaaaagcacttaaagcctataaataggcagtttttgagaattaaagcatctttaatgaggttgaacaaaatgcagtcttgacgctgaacaggactttactggaacgtagagctaaatggttcgatttcatgaaccagttacaaaaaaacccgctcatcggcgggtttataaaactttggcaacatatcaaatatgcttcaaatatggcttattttgttgcattttgcaagcgtgtttgaaggagatggtgaaatttacttcacatttctgccactttgagggcttcttcttcctcatagtattcaagagccatggccaacgcagattcatcaagctgggtaaaagcggcctttaacccagcccagtgccctgaatagacacgcaaccatgtcgaacggtcaacgctaaccatgcgggccaacgctgcaccagcatagtctttataggtttcattatttctggttgcggcaatttcctgccctgccagccataccaggcctatcagtttctttactacgcgctcctgaagggagttatcacccaggcatttctgataagttttccagacgtattcacacatcatcacctggtgcttatagctaaggtcaaaaccgtagcagtaccgcaaccaggcctgctggtatccactaagcgcggacactgccctacgccacggcgcggactcaaattccgcatcttttatcggcggcattggcctgcggcggctgcgtgtttccagcacatacagtggcgcggaaagtgagttaacaaagcgtggccccttctctccttcgagttcgacgagatgaattccacggcgaggggtggcatttttgtctgctggtgggtgttcactgaaagcctcaagctgcccttttgttcccccagacaggtcaggtagcgcgcggcgcaattctattcttacaaaattcaggtcttgttgattcatgcttctttgcgctccatacacttaagctttcgcaattacgccgatcgccagcgcccgatccataaaacgcagtagcagctcaagctgcgtaccatgcttctgctcgaatgccggtacatcggcgtgtaactcgtcgtggcactctctgcacagagggatcacgaagagatcatgggcttttgttgctgtcccccccataccgtgccctacgatatggtgcggatcatctgctggccgtcggcaacactcacagggttgtgttttaacccagcgggtgtacgtctcatttatccagcggcgtcgctttggcctgagcatgaaagattctggagactccggatcaacagagagcgtgaggatcttcttcgccttctcctgcacgaggctggttgcagaagcggaaggcacaatgtcgctttccctcatgaccgagcggatcttatcatccggaaggcgtagccccttgtgcgcaacgctttccggaataacatcagccaggtcgtttctgaccatccaccagcacagttccggaagcgtcaggatatgcgactcgggaaaaccagaatcacgccgaatgacttccagaatccaggataccaggtttcctgccgctatacctgcaagctgttcggtatgctgccccgacaaagtgtgatcgcaatgccagcacaggcgaatacttcctggtgggtgccgcattgttgtgaagttcttgtcgtgccacgatgaatgtggccactggcattcaaaccgattactcaaccattgctcaagggaaggaagcccaccagcacgctgaataacccgatcattctcgaagacctgccgcattaccggatcatcagccagcggctgaatggcggcgggaacagctcctgtactgaatgacgccatttcttctggttcaggctcaagcagaacgcgaccgcgcataaagaggtgcatcagttccgcgccgggacgaaacaacacaatccccatacgatgggcgatctcgggggtaagcagagctctcacgcgacctgccccctggcaatgtgttctgcccacagtccaccaatccagcgcacgcctttcgccgtgaaacgtgcctggctgaatgcatgatttgaggttacggatgtgccggttttcacttcaaaacggcccgcatcaatatgctgatgccgtggggtcatcgttccgccaagacgatacatgatgtcgttctcaaggaggaataaccgcagatcgggctctttggccttaagcagttttgccacctggcggaatgacattgacccactggctgtacagtaccgatcaacaaacgctaccttcggcgccgcggcagccagttcgttagtcaactgctgtttttgttctgcaaggtcagctgcaagacgtagggcttcagagaatgattgaggaatcgtctgctgctgtgcctgctcaagctcctgccagcgatcaaccagacgcgcggtaaactccggcgacagctgcgcgacaacgatataactgtcccgcttccctatcagataaaccgataccgactgattgaggtgatttttaacttcccccattggggggagttcaataacaccgcgctctgccaggcgttcaatggaccgtttaacatggtcatgtcttgattccaccagctcagcaatatcgctgctggacatggttaacgctgttgttgctaactggctcatacttttctccatatcaggcggctgcacccgccggttcatatctgctgattgttatctctacccgacctttcggcacaacgggtccccattccaccagcatgcgcttaatctggctgtcgtcttcccagacacccgcatgcgtcagcgcgtcaaacagggctttgttgtaattatcgatatcccggcggcgcgcatccggcgggtacagagtgatttctaccgctgccagttcagtcgatggcttcgggagacgtcgtaattgctcaatgatcgccacgcaggcagcgctctggtatttacggccagcggcgctaatgaggtgacgaccggccagcggccccttgttaggggcgcgccagtaagtgttcacgctcggaggaaaaggcaggatcagtttcacgcggcctctccccgcatattgcgaacaagttcagaagcagcagtaatgatttcgctggtggcagtccgctccagccagagttgattgatattggctttcagcttgtgttgcagtgactcatccagcatgtcagcaccatccacctggtcgaatacaattctaacctccagcggccagatacgggactcgggaagcggatccgatactggtttagctttctcacgaatgtgcatgcggatctggcgaatattggaccaactggaaacatccaggcttcccatggctgcaatgaagtcagtgctgttcatgccatattcaccggatgcttcaagggcaacagtgcgaatacgttccgacatatccaggcgcacagcagcgtcatcgaattcaatcgacaacagccactcatccacaccgaacaaaatactctcacgaataagcagcttcgctttgtcgatcgttaaaggtgatacctgagtgaattccggtgcttcgacagaatccgccgcccaggtatgcccaaacttcgattcactgaatgtgtattcttctttatcgccaaacgcagctctaacgcatgcccacgcctcgacaccgctgatagcaaaaatatctttctgggtgagtggcaactctgcttctggcttatcagctgcaggaggtgtggcagttacaggttgagacttgctggcagcaaattgtgccaaagccataaacgcccgcccttttgcctccagttctgtgcggttgatatagctgaaccgctcgccacgccatgacttatcgaatacagctatggcaccggcaaaaaacgcgctggtgggtttctgtttttcgtcagcaggtacaaaccacacaggcagatcgaacccaatgcgcccgcgaatgaatacaatgtgatcggcatcttccggccaccacgtttcactcggcgcggcttttatcaggaatacatagcgaccgcccttctcgcgctgggctgctgcgtagttcatgatgtgcgtcatgccggtgatcgcctgcttctcgtggtactgcgaacggctatacggtgggttgccatagccagcgccacccagttcagccagacgttcagaccagtcctgcgtcagcgcgttatcttcggcggtgtaccatgccgggcatttcgcgttgtcgtcgtcagcaaacaaatccagaactaatggaccaaatagcgcgttgatcccccaaaaaagcagatccggtgtccgccactgatcgccaacctctttcaattcgtgagctggtttgctacgcagtgccgccagcgcctggcaatatttattggtcatcatgaacggaaccccgaattttctggcagtgagtaatcaacactctggaagtttgcgcggctggctgagttagtctcccatttgccgttaacgcgttcaggccggccagcactggaccatttggtcgcgctttgcaggtaaccagggaagttttttggaatgaacagagttgccgggcggaggtattgcgcctgctcgctatcacgccaatcggcatttttgtaatccactaccaagcacaggtcatcaacagtgaattgttcccgaagacgggcgcgaatattctccagcgacgtgctgcatacctggtagcgtgagccagtagtctgattcaggtaagacaaaacctgtctggcctgatcagtaatcacaacctcagggtcgggttgcgccgcaaccggacaagagggttttgaagttacttgtggttcttgttttgattttactgacggatccccaccagattctgacgggtcaaaaccgccttttttgctggatttcgacgcctcaaattttgacgggtcggattttgatgcatcagattttgatgggtcagattttgatgcgtcagattctgacaggtgagaaaaagcagcctctcgcaacttaacgacgttaagctgatatacgttcgatgcgttacggttcccattacggcgctgcttacgggaaagccagccatccttctcaagctgagcaagggccgtccttaccgtactttctccagcaccgatttgacgtgcgatcgtgccaatagacggccagctaacaccctcatcactactgaagtcggccagacgcgccatgatggcaacgctggataacttcatgcctgacgaagcgcatgcatcccatacgtaaccggttaatttagtgctcatggtcgtcctttaattctgtaaatttacgctggaattgttcaagagggctgaagcactcatgatcgtacccttcgcgaaggtatataacgcgctgtgtatctggctcccagcggacaactctgacgggaactccgtagtgatctctgaaccgccggttaacttcagccattcctcgcgccccttctcgttcatctgaacaaatgcttctaccatcaagtctgctggctggtagttgcctccatcagccgcgttatttatgatttccacatagccgaactgggcatctttacccaccagcggcaaacatctgaattgcttagctggtctgaatcggtttacactgttcatgcgttagtttctccactgatacgacacgccaaggcgcccggagctgcacactcgcgggcgtcaccttttctgcctgttgaaacgaatacgtcaatcgcctgatctgaaacaccaaccccataaagcgccataaatcccaggaacccgtgaatctggtggcggagcttcttactgaataattctgaaagcattttgcgctctgatgaatcaattaccccatcagccgctgctgccatcttggcattagccagctcaccagatgctgctgccgctttcatctcaatgctgtacagctcaacgttatccaggctctcagcagttggaacatccaccagccatttcccttttcggttcgcctggtactcggccaagtaacaagaaccagacaggtcctccatccgttccagttctgccaaggtaaagaaccgactgccacacttctggtacaggtggttgtggaactggtcgatagtcatccctaaatcggaagccatacctaagcgaccatgcttgtgtgccttacacatcaggcggattgctgtatttatgctgtctaccatgttgatttccctctggtagttaataatcaacttaaagttgactattgttgttagcggaaggtatgccgtcatttttgttcggataaatatcaggtcgtaattgatggggagttactacccatccgccccattggcagagttgaataactctttcagaaggtactcggttctttgcaatccagttcgcaacagattgaactgattggaattcaaaccgccttgatacctctgaaatcgacccgatcgccttcacagctttagctgttacattcttgtgttgagatgacatgtgttctcctatgactaagcctgcatcaatactacttatagtagcaattattagcaacttaaaatagaaatgacaactatgccttgtgcgcttaatcttctacttatggtggaaaatgctaaatacaaagactttgccgaaaggctaaacaggtctctccaagagcaatctattggagttaaagaattgtcagagttcagtggtgtctcgtatgagatggcgcggcgctacactcttggtactgcaaagccgagagatgagaagatgattcgaattgcagaaagacttgccgtctcaccggcttatcttgattatggtgtgcctgttaatggtggcgacgcgccagccaaaggcacggtcagaatagagcaattggatgttcatgcttcagccggttccggatatataaaccaaccattccctacaatagtgagctcaatagagattccagaagagaggatcttcgagttgtttggtcgtagaagccttgatggcatcgtcatgataaatgttgatggcgatagcatgatgcccacgctttgcccaaaggacctgcttttcatagacagcaaggttgaacaattcagcggcgacggcgtttatgtgttcaattttgaagacagtacgttcgttaaacgtttgcagaaggtaaaagggcgccgactggcagttctttcagacaatgaacattacccgcccttcttcatagaggagcatgaaatgaatgaactatacatattcggcaagctaatcagatgcttacctctaaaaatgatagagtttggctaataattaattcatcaagaaaccggcgaaagccggttttttttacgcctccaattcctcacctcataacactacactactaaaaatttcattttctactttttgttgttgcaattatctacttaaagtagctatagtcattgcatcgaaagcgaacaggcaggacgcccacgaagtagccgccggtggcatatgaataaccggatgattcgctgacagaaaacttaggttgggggtagaggtttacatgaatcatttattcacatgctcattttgcggagcaaccgaactgggagcgataaagatcgtcgcaaaaggtggtaaggacgaacctgccatctgttcggaatgcgtagtcacatgtgtagaaaaaatgatcctgactaaaaaatcagaggctgaaaaaccaacctctgataacgaaataatatcagtcgataaaaaactatttaaagagcttcttcagcttgtcctcaaccttcctgatttcggaagtaagctggctgctgttgacattgatagtagctccacatcgacaagtgaaacttttgttcgacttgagccaagcgattttcttcttcgtcttagtgccgcacttagggcatgcgggtaacgtaatttcctggttatcaaaagcgcccataaacatccctcttggttgtgtgagaacaccaagataccaccgcgcctgatgtggttaaaagcaggctaaagcaataacaagtaactccctgttctggcggcccggtgttttcccgtgtatttccggtaaccgccagcctttttcagggcacaacagaaaagggcatcaccgggcgacgggctcataacccaatccacccgggcaaaaagaaagcggtctctgcaagccgccgaccaatgcaggtgcccttctctgttgtgtatggagaaactaactttttagcgtctgtgcagatgcgctgaggaaccgagaatgaataatccgtttttcaaaaatatgttggtgtatcgcattagtcgcgatttcaccatcaaccaggaagagctggaacagcagcttgaactatttcgcttcactccatgcggtagccaggatatggcaaaaaccggttgggtatcaccacttggtcagctgtcagatcgcttgcatcacactgtcaataatcaagtgttgttggttattcgccgggaagaaaaaatactgccatctcctgtcattactgaagaactgcgcaagcgtgtgtcgcgtctagaatccgatcaggggcgtcgcctcaaaaaaactgagaaagattcgctgcgtgatgaagtgttgcactccctgcttcctcgggcgttctccaaaaactcgactgttggtttgtggatcaacgtcaccgacggtctgatcatggttgatgcagccagcgctaaacgtgccgaagactcactggccctgcttcgtaaaactctcggttctctcccggtggtaccgctgactatggaaacgccgatcgaactaactatgaccgactgggttcgttccggtagtgcgcctgctggctttggcctgggtgatgaagccgaactgaaagctattcttgaagatggcggtattggacgctttaaaaaacagactctggtcagtgacgaaattcatgtgcatctggaagctggcaaagtagttacaaagctgtctatcgactggcaacagcgcattcagttcgttctttgcgatgacggcagcatcaaacgccttaagttctctaatgagattacagaacaaaacgacgatatcgaccgtgaggatgcggctcagcggttcgacgctgactttgttctgatgaccggcgagcttatctctctcattaacggattaacaacctctctcggcggcgaagccaagcgataaacaccaggcaacaattacccccataagcatgggttgggttgctgcacgctaaattcagcaattcattaatttaatggcgcggtgcagcgcgccaatatggagaaaaccatgagctacattcagacattatccggcaaacattttaattacctcgatatccaacaggacgatatcgtgatcgaggatattgctaccgcgttgtctcatatctgccgctttgcagggcatcttcctgagttttacagtgtcggccagcatagcgttttaaccagccacctcgttccgcaggagtttgcattagaagcactgcttcatgatgctgctgaagcctacctgcaggacatcccctccccacttaagcgcctgcttccggattaccaggcaatcgaagctcgtgtggacgcagccattcggcagaagttcggtctaccaactgagcaacacccaaccgtgaaatatgccgacctggtgatgctcgccagcgaacgccgcgattttgagattgacgaaggttccatttggccatgcctcgagggagttgtcccaacggatttattcattatcaacccagttcgtcctggccagtcatacggcatgttcatcaatcgctttaacgagttgatggagcagcgccaatgcgccgcatgaaggtaaaagaactcgtagcggaggcgtttgcctccgttgctgaattgccaccaaagcatgcgccgcttatgcgcgaagtcgccaccagactggacgctacgttcgcagcattaaaagagtctctggtgcaactggaacaggaacgtaaagataaaacgccatgaccgtatttgaatatctccaggctcatccgaataccaccagcggtgaaatcgccaaaggtatgaacaaaaagaccccagcggtcgccggagcattatctcagctctatggcaccggtcggatcgtgaagtctggtgttcgcaagggtattccaacataccgcattaacgatatgccgtttggttgcagtaacagcctaaccatgatgtttaaccagctcttgagcagagccagacaaggagcagcccaatgacagcactcaacaaacaggcgctgcgtgaagaattccagttcatgcaggacaactatagcgacccggcagaccacgatcggcaggtgatttacatcgaggcggaggcgctgctggatgagttggaagccaaagactcaacgatagcagcacaacaacatgagatccgtatgttgctgaatgcgcttgaggaaaaaccatgcccgaaatgcaacgacacaggaatgactgatagtggcggcacgcagccatggggcgagccgattgagattgaatgcgactgccgacagcaggatgccaacaccgcagaacttgtagccgctggcattggcgtgaagggggagtgagatggataaattaatcaaacctaccgccaaaggtaaatatgacggttcatgtgattatctttgctcggaagatgcgcgattcatcgttatgcgcggcgattatacggaagcggaaataattcaggcttctgtgtctcaagatgtaatcgactcggatggtgcggctgattttgcaagtagcgcccgctattatcagtgctggtacaaagttagcccaataggtggtcaggatggctattcaggctggcatcatcctcgtgattcgccgtgtcgcggtgcatatttcgcatcagttttgcaatgggattaaggaggactaacccatgacaactaacaaccacccggcgcacggtcctgtatcactcgatcgcctgcaccagatacgcgaacacctgctgcatgatacccaatactcaaacggcgggaacagagcctacattctcgctgatgtattgaaggtgattgatggggctattgcccgcgagctggtacgccgtgagcatgcagcgtggtcacaggctactttcggcgatgtcggtccagttggtccgctgaagcacctttccaaagaagcgctcgaggctgctgctgaaccaggcgaccttagcgaatgggctgacatgcaattcctgttatgggatgcgcaacgtcgtgccggtatcagtgatgagcagattacccaggcaatgataaaaaagctggctataaataaggttcgccaatggcctgagccgaaagacggggaacctcgattgcatatcaaagaacagtcagagcaggagaaaaaataagaatgtttagcctgattcggcgcggtcaaatctacacggacagtagcaactggcccgtaattatccatagctgtagtgatcactcggtccgaattaaacgcaatgatggcgagctgagaacgattagcatcaaacgctttaacgaagattttgaacgagtggagcatgatgagtatcgcaaaatatgtgccgaaatagagcaggaaacaaacctgaaaaacctacgtgcgatgcgtcgcggcaagattactgaatagccaaacaggagaatatttaacgtgaacaacttaatgatcgaccttgagtccatgggcaaaaaaccgaatgcccctattgtctccattggtgccgtattcttcgatccgcaaagcggtgaactgggtcaggagttttacaccgctgttaatcttgaaagcgctatggagcagggagcggtgccggatggtgacactattctgtggtggttaagacaaagctcagaagcacgatcagcaatctgtgttgatgatgcgatgccgatatcatctgccctatctgaactgagccatttcattaatcggcattctgataaccctaaatatttaaaagtttggggcaatggagctactttcgacaacgttatattgcgcggcgcatatgagcgtgccggccaggtttgcccgtggcaattttggaatgatcacgacgtcagaaccatcgtcacattaggcagatctgtaggtttcgatcctaagcgtgatatgccatttgatggggttgcacataacgcactggctgatgcccgccaccaggcgaaatatgtttcagcgatttggcagaaactaatcccaaccaccagcaacagctaaagttttccccgggtgcagccgggataatggagaaataactatgagcaatattttccagttagctcccaacgattgggtttgtgaaagcgttttgatcgcggttactgggctcaaacccggaaccatcctccgtgccagaaaagaatgctggatgattgggagggagtatatccacgtatcgcctgacggaaatcctaaaccttccagtgagtgcatgtataacagaaaggctgtagatgcctgggtcgcttcaatgaaaagcaagcaaccagggtgatttgatgccatgaaaaaggtaagctcgtatcgctcttgggcgtctggaggtaacaccaatggataaagtcacatatccaacaggcgtcgaaaaccacggtggcacattacgcatctggtttaattttaaaggtaagcgtgtcagggaaagtctcggtgtccctgacaccgctaagaacaggaagatagccggggaactgcggacatcagtatgttttgccatccgcacaggaacctttgattatgcaacccagtttcctgactcccctaacctcaaggcttttggtgtaagtaaaaaagacattacagtgaaagaacttgaagaaaaatggctggatctgaaacggatggaaatctgcgcgaacgcatttaatcgctatgaatctgtcgcaaggaatatggtgccgaggatcggaggtaatcgcctggtgtcagcagtaaccaaagaggaattgctgtatctgaggaaatatttgctaactggttatcagaatccgacgaaaaacaaagccccggcaaaagggcgaagcgttgttactgtgaactattacatgacgacaatggccggaatgtttcagtttgctgcggatcacggttacttagaggtgaacccattcgagggaattaagcctctgaaaaaagccagggcagaaccagatcctctgtctcgtgatgaatttattcgcctgatagatgcatgccggcatcagcagacgaaaaacctgtggtcattagcagtgtacacaggaatgcgtcacggggaactggtctccctggcctgggaagatatcgacctgaaggctggaacaattaccgtcagacgtaattatacgaaacttggtgagttcactctaccgaaaaccgaggcaagcacagatcgagtggtgcatcttatccagcccgcaatcagtatcctgaaaaatcaggctgaaatgacaaggctgggcaggcaatatcacattgaagtgcagttacgtgagtacggccgttcggtgaaccatgagtgtacattcgtctttaatccgcatgtggtcagacgcagtaagcaggtcggatttatctaccgggtcgattcagtaggcgactcatgggaagcggcacttaagcgtgcggggatcagacacagaaaggcgtaccagtcacgacacacctatgcgtgctggtcattatcagctggtgcaaaccctagttttattgccagtcagatggggcatgcgagcgcgcagatggtgttcaatgtttacggtgcatggatggctgacagcagcgcagagcagatcgcaatgctgaatcagaagctggcagattttgccccattgatgccccatagccacgagaacagtacgggaggattattaaaatcagtaagttaacccctaacgcccgtcatgttaactgtgtggagggtaacaccacgctttatgccctgccgaaacccgaggttgtcctgcgctggcgtgagcagaccacagatgacttccgcttctgttttaagtttccggcgaccatttcgcatcaggcagcattacggcattgcgatgatttagtgactgaatttttgacccgcatgtcaccgttggctccgcgcattggacaatactggctgcaactgcctgccacattcggcccacgggagctgcctgcgctttggcattttctcgattctcttcccggtgaatttaattatggggtggaagtccgccatccacagtttttcgccaaaggggaagaggaacaaacgcttaatcgcggtttacatcagcgcggcgttaatcgggtgattttagacagccgcccggttcatgcagcacgtccatacagtgaagctattcgcgacgctcaacgaaaaaaacctaaagttccggtacatgctgtactgacggcgaaaaatccactgatccgttttatcggtagtgatgatatgacgcaaaaccgggaattatttcaggtctggttacaaaaattagcgcagtggcatcagaccactacgccttatctttttttacatacgccagatattgcccaggccccggaactggtacataccctgtgggaagacttacgtaaaacgcttccagagatcggagcagttccggctattccacagcaatcttctcttttctgaatttgccacctatcatagacaggtgccatcggccattttaaagggagtttgtatggtaagcgcgctgtatgccgttttaagtgcgttgttattaatgaagttctcttttgatgtcgttcgcctgcgaatgcagtaccgcgttgcctatggcgacggcggttttagcgaactgcaaagcgctattcgcattcatggtaacgcggtggaatatattcctatcgcgattgtgttgatgctgtttatggaaatgaatggcgcagaaacctggatggtgcatatttgcggcatcgttttgcttgctggtcgtctgatgcattattacggttttcatcaccgtctgttccgctggcgacgttctggcatgagcgccacctggtgtgcgctgttgctgatggtgctggcgaatctttggtatatgccctgggagttggttttctccctgcgttagcgcacaatacgccactttctttttcccggatttttacgttatgtctcaccgcgacacgctattttctgcccctatcgccagactgggcgactggacctttgatgaacgggtagctgaagtcttcccggatatgatccagcgttccgttcccggctattccaatattatttccatgattggtatgttagccgagcgcttcgttcaacctggtacgcaggtttacgatctgggttgttctctgggcgcggcgacgctctcggtgcgtcgcaacattcatcatgataattgcaaaattattgccatcgacaactccccggcgatgattgaacgctgccgtcgtcatattgacgcctataaagcccctacgccagtagacgttattgaaggtgatattcgcgatatcgccattgaaaacgcatcgatggtggtgctgaattttaccctgcaattcctggaaccttccgagcgccaggcgttactggataaaatttatcaagggctgaacccgggcggtgcgctggtgctttcggaaaaattcagtttcgaagatgccaaagttggtgaactgctgttcaacatgcaccacgactttaaacgtgccaacggttacagcgaactggagatcagccagaaacgcagcatgctggaaaacgtgatgctgaccgattccgtggaaacccataaagcacgcctgcataaagccggttttgagcatagcgagctgtggttccagtgctttaactttggttcactggtggcattaaaagcagaggacgctgcatgatcgactttggtaacttttattctctgattgccaaaaatcatctttcacactggctcgaaacgctgcccgcgcagattgctaactggcagcgcgagcagcagcacgggctgtttaagcagtggtccaacgcggtggaatttctgcctgaaattaaaccgtatcgtctggatttattgcatagcgtaaccgccgaaagcgaagagccactgagcgccgggcaaattaagcgcattgaaacgctgatgcgcaacctgatgccgtggcgcaaagggccgttctcactgtatggcgtcaacatcgataccgaatggcgttccgactggaaatgggatcgcgttatgccccatctttctgatttaaccgggcgcaccattcttgatgtcggctgtggcagcggttatcacatgtggcgcatgattggcgcaggggcgcatctggcggtgggtatcgatcccacgcagctattcctctgccagtttgaagcagtgcgtaaactgctgggtaacgatcagcgcgcacatttgttaccgttaggtattgaacaacttccggcactgaaagcctttgataccgtcttttcgatgggcgtgctttatcatcgtcgttcaccgctggagcatctctggcagttaaaagaccaactggtgaatgaaggcgaactggtgctggaaacgctggttattgatggcgacgaaaacacggtgctggtgccgggcgatcgttacgctcaaatgcgtaatgtctatttcattccttccgcgctggcgctgaaaaactggctgaagaagtgtggttttgttgatattcgcattgcagatgtgagcgttaccaccacagaagagcagcgacgcaccgaatggatggtcaccgagtctctggccgattttctcgacccgcatgatccgggtaaaacggtggaaggttatcctgcgcctaaacgcgcggtgctgattgcgcgcaagccgtaaaggtctggtaatactgccggatgcggcgtgaacgccttatccggcctacaaagtcttgctaattcaatatattgcaggggctatgtaggcctgataagcatagcgcatcaggca

SEQ ID NO: 282 - DNA, 290 bp, Escherichia coli
  source 1..290: mol_type = genomic DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 283 - DNA, 173 bp, Escherichia coli; identical to SEQ ID NO: 11
  source 1..173: mol_type = genomic DNA

SEQ ID NO: 284 - DNA, 305 bp, Escherichia coli; identical to SEQ ID NO: 12
  source 1..305: mol_type = genomic DNA

SEQ ID NO: 285 - DNA, 180 bp, Escherichia coli; identical to SEQ ID NO: 13
  source 1..180: mol_type = genomic DNA

SEQ ID NO: 286 - DNA, 199 bp, Escherichia coli; identical to SEQ ID NO: 14
  source 1..199: mol_type = genomic DNA

SEQ ID NO: 287 - DNA, 117 bp, Escherichia coli
  source 1..117: mol_type = genomic DNA
atccccatcactcttgatggagatcaattccccaagctgctagagcgttaccttgcccttaaacattagcaatgtcgatttatcagagggccgacaggctcccacaggagaaaaccg

SEQ ID NO: 288 - DNA, 108 bp, Escherichia coli
  source 1..108: mol_type = genomic DNA
ctcttgatcgttatcaattcccacgctgtttcagagcgttaccttgcccttaaacattagcaatgtcgatttatcagagggccgacaggctcccacaggagaaaaccg

SEQ ID NO: 289 - DNA, 290 bp, Escherichia coli; identical to SEQ ID NO: 282
  source 1..290: mol_type = genomic DNA

SEQ ID NO: 290 - DNA, 1485 bp, synthetic construct
  misc_feature 1..1485: note = Synthetic polynucleotide
  source 1..1485: mol_type = other DNA
ctcttgatcgttatcaattcccacgctgtttcagagcgttaccttgcccttaaacattagcaatgtcgatttatcagagggccgacaggctcccacaggagaaaaccgatgaaaaacgcgtcaaccgtatcggaagatactgcgtcgaatcaagagccgacgcttcatcgcggattacataaccgtcatattcaactgattgcgttgggtggcgcaattggtactggtctgtttcttggcattggcccggcgattcagatggcgggtccggctgtattgctgggctacggcgtcgccgggatcatcgctttcctgattatgcgccagcttggcgaaatggtggttgaggagccggtatccggttcatttgcccactttgcctataaatactggggaccgtttgcgggcttcctctctggctggaactactgggtaatgttcgtgctggtgggaatggcagagctgaccgctgcgggcatctatatgcagtactggttcccggatgttccaacgtggatttgggctgccgccttctttattatcatcaacgccgttaacctggtgaacgtgcgcttatatggcgaaaccgagttctggtttgcgttgattaaagtgctggcaatcatcggtatgatcggctttggcctgtggctgctgttttctggtcacggcggcgagaaagccagtatcgacaacctctggcgctacggtggtttcttcgccaccggctggaatgggctgattttgtcgctggcggtaattatgttctccttcggcggtctggagctgattgggattactgccgctgaagcgcgcgatccggaaaaaagcattccaaaagcggtaaatcaggtggtgtatcgcatcctgctgttttacatcggttcactggtggttttactggcgctctatccgtgggtggaagtgaaatccaacagtagcccgtttgtgatgattttccataatctcgacagcaacgtggtagcttctgcgctgaacttcgtcattctggtagcatcgctgtcagtgtataacagcggggtttactctaacagccgcatgctgtttggcctttctgtgcagggtaatgcgccgaagtttttgactcgcgtcagccgtcgcggtgtgccgattaactcgctgatgctttccggagcgatcacttcgctggtggtgttaatcaactatctgctgccgcaaaaagcgtttggtctgctgatggcgctggtggtagcaacgctgctgttgaactggattatgatctgtctggcgcatctgcgttttcgtgcagcgatgcgacgtcaggggcgtgaaacacagtttaaggcgctgctctatccgttcggcaactatctctgcattgccttcctcggcatgattttgctgctgatgtgcacgatggatgatatgcgcttgtcagcgatcctgctgccggtgtggattgtattcctgtttatggcatttaaaacgctgcgtcggaaataa

SEQ ID NO: 291 - DNA, 4323 bp, synthetic construct
  misc_feature 1..4323: note = Synthetic polynucleotide
  source 1..4323: mol_type = other DNA
ctcttgatcgttatcaattcccacgctgtttcagagcgttaccttgcccttaaacattagcaatgtcgatttatcagagggccgacaggctcccacaggagaaaaccgatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccctcgccacagaacaatacaatcaagatattgtcagtttaggtctgcatgccgctcaagatgttttagagatggagcagaaattacgcaatattgtttcaatgacaattctggtagtttgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgtt

SEQ ID NO: 292 - DNA, 5092 bp, synthetic construct
  misc_feature 1..5092: note = Synthetic polynucleotide
  source 1..5092: mol_type = other DNA
ctctagaaataattttgtttaactttaagaaggagatatacatatgaaaacactatcacaggcccaatctaaaacttcttcacagcaattcagctttaccgggaactcgtctgcgaatgtaattatcggcaatcaaaagctgaccattaatgatgtagctcgcgttgcccggaatggcactttggtgtcactgacgaacaataccgacattctgcaaggtattcaagctagctgcgattatatcaataacgccgttgaatctggcgagccaatctacggggtaacaagcggttttggtgggatggcgaacgttgccattagccgtgaacaggcgagcgaacttcagaccaacctcgtttggttcctaaagacaggagctggtaataagttacctctggctgacgtaagagccgcgatgctgcttcgcgctaatagtcacatgcgcggcgccagtggtatccgtcttgagcttatcaagaggatggaaatcttcctcaacgcgggtgtcacaccatatgtttatgagtttggtagtatcggagccagtggtgatcttgttcccctgagttatattacgggttcattgattggtttagacccgtcctttaaagtggattttaacgggaaagaaatggacgccccgaccgctttacgacagcttaatctgagcccacttactttgctccctaaagaaggtcttgccatgatgaatggcacctctgtgatgactggaattgccgcgaattgtgtgtatgacacgcagatcctaacggccattgccatgggtgttcacgcgttggacattcaagccctgaatggtacaaaccagtcgtttcatccgtttatccataattcaaaaccccatccgggacagctttgggctgctgatcagatgatctcactcctggccaatagtcaactggttcgggacgagctcgacggcaaacatgattatcgcgatcatgagctcatccaggaccggtattcacttcgttgtctcccacaatacctggggcctatcgttgatggtatatctcaaattgcgaagcaaattgaaattgagatcaatagcgtaaccgacaacccgcttatcgatgttgataatcaggcctcttatcacggtggcaattttctgggccagtatgttggtatggggatggatcacctgcggtactatattgggcttctggctaaacatcttgatgtgcagattgccttattagcttcaccagaattttcaaatggactgccgccatcattgctcggtaacagagaaaggaaagtaaatatgggccttaagggccttcagatatgtggtaactcaatcatgcccctcctgaccttttatgggaactcaattgctgatcgttttccgacacatgctgaacagtttaaccaaaacattaactcacagggctatacatccgcgacgttagcgcgtcggtccgtggatatcttccagaattatgttgctatcgctctgatgttcggcgtacaggccgttgatttgcgcacttataaaaaaaccggtcactacgatgctcgggcttgcctgtcgcctgccaccgagcggctttatagcgccgtacgtcatgttgtgggtcagaaaccgacgtcggaccgcccctatatttggaatgataatgaacaagggctggatgaacacatcgcccggatatctgccgatattgccgccggaggtgtcatcgtccaggcggtacaagacatacttccttgcctgcattaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaa

SEQ ID NO: 293 - DNA, 4987 bp, synthetic construct
  misc_feature 1..4987: note = Synthetic polynucleotide
  source 1..4987: mol_type = other DNA
ctctagaaataattttgtttaactttaagaaggagatatacatatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccctcgccacagaacaatacaatcaagatattgtcagtttaggtctgcatgccgctcaagatgttttagagatggagcagaaattacgcaatattgtttcaatgacaattctggtagtttgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaa

SEQ ID NO: 294 - DNA, 5962 bp, synthetic construct
  misc_feature 1..5962: note = Synthetic polynucleotide
  source 1..5962: mol_type = other DNA
ctcttgatcgttatcaattcccacgctgtttcagagcgttaccttgcccttaaacattagcaatgtcgatttatcagagggccgacaggctcccacaggagaaaaccgatgaaaacactatcacaggcccaatctaaaacttcttcacagcaattcagctttaccgggaactcgtctgcgaatgtaattatcggcaatcaaaagctgaccattaatgatgtagctcgcgttgcccggaatggcactttggtgtcactgacgaacaataccgacattctgcaaggtattcaagctagctgcgattatatcaataacgccgttgaatctggcgagccaatctacggggtaacaagcggttttggtgggatggcgaacgttgccattagccgtgaacaggcgagcgaacttcagaccaacctcgtttggttcctaaagacaggagctggtaataagttacctctggctgacgtaagagccgcgatgctgcttcgcgctaatagtcacatgcgcggcgccagtggtatccgtcttgagcttatcaagaggatggaaatcttcctcaacgcgggtgtcacaccatatgtttatgagtttggtagtatcggagccagtggtgatcttgttcccctgagttatattacgggttcattgattggtttagacccgtcctttaaagtggattttaacgggaaagaaatggacgccccgaccgctttacgacagcttaatctgagcccacttactttgctccctaaagaaggtcttgccatgatgaatggcacctctgtgatgactggaattgccgcgaattgtgtgtatgacacgcagatcctaacggccattgccatgggtgttcacgcgttggacattcaagccctgaatggtacaaaccagtcgtttcatccgtttatccataattcaaaaccccatccgggacagctttgggctgctgatcagatgatctcactcctggccaatagtcaactggttcgggacgagctcgacggcaaacatgattatcgcgatcatgagctcatccaggaccggtattcacttcgttgtctcccacaatacctggggcctatcgttgatggtatatctcaaattgcgaagcaaattgaaattgagatcaatagcgtaaccgacaacccgcttatcgatgttgataatcaggcctcttatcacggtggcaattttctgggccagtatgttggtatggggatggatcacctgcggtactatattgggcttctggctaaacatcttgatgtgcagattgccttattagcttcaccagaattttcaaatggactgccgccatcattgctcggtaacagagaaaggaaagtaaatatgggccttaagggccttcagatatgtggtaactcaatcatgcccctcctgaccttttatgggaactcaattgctgatcgttttccgacacatgctgaacagtttaaccaaaacattaactcacagggctatacatccgcgacgttagcgcgtcggtccgtggatatcttccagaattatgttgctatcgctctgatgttcggcgtacaggccgttgatttgcgcacttataaaaaaaccggtcactacgatgctcgggcttgcctgtcgcctgccaccgagcggctttatagcgccgtacgtcatgttgtgggtcagaaaccgacgtcggaccgcccctatatttggaatgataatgaacaagggctggatgaacacatcgcccggatatctgccgatattgccgccggaggtgtcatcgtccaggcggtacaagacatacttccttgcctgcattaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcg

SEQ ID NO: 295 - DNA, 5857 bp, synthetic construct
  misc_feature 1..5857: note = Synthetic polynucleotide
  source 1..5857: mol_type = other DNA
ctcttgatcgttatcaattcccacgctgtttcagagcgttaccttgcccttaaacattagcaatgtcgatttatcagagggccgacaggctcccacaggagaaaaccgatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccctcgccacagaacaatacaatcaagatattgtcagtttaggtctgcatgccgctcaagatgttttagagatggagcagaaattacgcaatattgtttcaatgacaattctggtagtttgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcg

SEQ ID NO: 296 - DNA, 6602 bp, synthetic construct
  misc_feature 1..6602: note = Synthetic polynucleotide
  source 1..6602: mol_type = other DNA
accactccctatcagtgatagagaaaagtgaactctagaaataattttgtttaactttaagaaggagatatacatatgaaaacactatcacaggcccaatctaaaacttcttcacagcaattcagctttaccgggaactcgtctgcgaatgtaattatcggcaatcaaaagctgaccattaatgatgtagctcgcgttgcccggaatggcactttggtgtcactgacgaacaataccgacattctgcaaggtattcaagctagctgcgattatatcaataacgccgttgaatctggcgagccaatctacggggtaacaagcggttttggtgggatggcgaacgttgccattagccgtgaacaggcgagcgaacttcagaccaacctcgtttggttcctaaagacaggagctggtaataagttacctctggctgacgtaagagccgcgatgctgcttcgcgctaatagtcacatgcgcggcgccagtggtatccgtcttgagcttatcaagaggatggaaatcttcctcaacgcgggtgtcacaccatatgtttatgagtttggtagtatcggagccagtggtgatcttgttcccctgagttatattacgggttcattgattggtttagacccgtcctttaaagtggattttaacgggaaagaaatggacgccccgaccgctttacgacagcttaatctgagcccacttactttgctccctaaagaaggtcttgccatgatgaatggcacctctgtgatgactggaattgccgcgaattgtgtgtatgacacgcagatcctaacggccattgccatgggtgttcacgcgttggacattcaagccctgaatggtacaaaccagtcgtttcatccgtttatccataattcaaaaccccatccgggacagctttgggctgctgatcagatgatctcactcctggccaatagtcaactggttcgggacgagctcgacggcaaacatgattatcgcgatcatgagctcatccaggaccggtattcacttcgttgtctcccacaatacctggggcctatcgttgatggtatatctcaaattgcgaagcaaattgaaattgagatcaatagcgtaaccgacaacccgcttatcgatgttgataatcaggcctcttatcacggtggcaattttctgggccagtatgttggtatggggatggatcacctgcggtactatattgggcttctggctaaacatcttgatgtgcagattgccttattagcttcaccagaattttcaaatggactgccgccatcattgctcggtaacagagaaaggaaagtaaatatgggccttaagggccttcagatatgtggtaactcaatcatgcccctcctgaccttttatgggaactcaattgctgatcgttttccgacacatgctgaacagtttaaccaaaacattaactcacagggctatacatccgcgacgttagcgcgtcggtccgtggatatcttccagaattatgttgctatcgctctgatgttcggcgtacaggccgttgatttgcgcacttataaaaaaaccggtcactacgatgctcgggcttgcctgtcgcctgccaccgagcggctttatagcgccgtacgtcatgttgtgggtcagaaaccgacgtcggaccgcccctatatttggaatgataatgaacaagggctggatgaacacatcgcccggatatctgccgatattgccgccggaggtgtcatcgtccaggcggtacaagacatacttccttgcctgcattaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttatttt

SEQ ID NO: 297 - DNA, 6497 bp, synthetic construct
  misc_feature 1..6497: note = Synthetic polynucleotide
  source 1..6497: mol_type = other DNA
accactccctatcagtgatagagaaaagtgaactctagaaataattttgtttaactttaagaaggagatatacatatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccctcgccacagaacaatacaatcaagatattgtcagtttaggtctgcatgccgctcaagatgttttagagatggagcagaaattacgcaatattgtttcaatgacaattctggtagtttgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttatttt

SEQ ID NO: 298 - DNA, 2137 bp, synthetic construct
  misc_feature 1..2137: note = Synthetic polynucleotide
  source 1..2137: mol_type = other DNA
ccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaaaacgcgtcaaccgtatcggaagatactgcgtcgaatcaagagccgacgcttcatcgcggattacataaccgtcatattcaactgattgcgttgggtggcgcaattggtactggtctgtttcttggcattggcccggcgattcagatggcgggtccggctgtattgctgggctacggcgtcgccgggatcatcgctttcctgattatgcgccagcttggcgaaatggtggttgaggagccggtatccggttcatttgcccactttgcctataaatactggggaccgtttgcgggcttcctctctggctggaactactgggtaatgttcgtgctggtgggaatggcagagctgaccgctgcgggcatctatatgcagtactggttcccggatgttccaacgtggatttgggctgccgccttctttattatcatcaacgccgttaacctggtgaacgtgcgcttatatggcgaaaccgagttctggtttgcgttgattaaagtgctggcaatcatcggtatgatcggctttggcctgtggctgctgttttctggtcacggcggcgagaaagccagtatcgacaacctctggcgctacggtggtttcttcgccaccggctggaatgggctgattttgtcgctggcggtaattatgttctccttcggcggtctggagctgattgggattactgccgctgaagcgcgcgatccggaaaaaagcattccaaaagcggtaaatcaggtggtgtatcgcatcctgctgttttacatcggttcactggtggttttactggcgctctatccgtgggtggaagtgaaatccaacagtagcccgtttgtgatgattttccataatctcgacagcaacgtggtagcttctgcgctgaacttcgtcattctggtagcatcgctgtcagtgtataacagcggggtttactctaacagccgcatgctgtttggcctttctgtgcagggtaatgcgccgaagtttttgactcgcgtcagccgtcgcggtgtgccgattaactcgctgatgctttccggagcgatcacttcgctggtggtgttaatcaactatctgctgccgcaaaaagcgtttggtctgctgatggcgctggtggtagcaacgctgctgttgaactggattatgatctgtctggcgcatctgcgttttcgtgcagcgatgcgacgtcaggggcgtgaaacacagtttaaggcgctgctctatccgttcggcaactatctctgcattgccttcctcggcatgattttgctgctgatgtgcacgatggatgatatgcgcttgtcagcgatcctgctgccggtgtggattgtattcctgtttatggcatttaaaacgctgcgtcggaaataa

SEQ ID NO: 299 - protein, 5 aa, synthetic construct
  REGION 1..5: note = Synthetic peptide
GGGGS

SEQ ID NO: 300 - protein, 18 aa, synthetic construct
  REGION 1..18: note = Synthetic peptide
KESGSVSSEQLAQFRSLD

SEQ ID NO: 301 - protein, 14 aa, synthetic construct
  REGION 1..14: note = Synthetic peptide
EGKSSGSGSESKST

SEQ ID NO: 302 - protein, 8 aa, synthetic construct
  REGION 1..8: note = Synthetic peptide
GGGGGGGG

SEQ ID NO: 303 - protein, 12 aa, synthetic construct
  REGION 1..12: note = Synthetic peptide
GSAGSAAGSGEF

SEQ ID NO: 304 - DNA, 22 bp, Escherichia coli
  source 1..22: mol_type = genomic DNA
ggaattgtgagcgctcacaatt

(19) United States
(12) Patent Application Publication
(10) Pub. No.: US 2024/0368531 A1
(43) Pub. Date: Nov. 7, 2024

(54) BACTERIA FOR THE TREATMENT OF DISORDERS

(71) Applicant: Synlogic Operating Company, Inc., Cambridge, MA (US)

(72) Inventors: Dean Falb, Sherborn, MA (US); Adam B. Fisher, Cambridge, MA (US); Vincent M. Isabella, Medford, MA (US); Jonathan W. Kotula, Berkeley, CA (US); David Lubkowicz, Somerville, MA (US); Paul F. Miller, Salem, CT (US); Yves Millet, Newton, MA (US); Sarah Elizabeth Rowe, Durham, NC (US)

(21) Appl. No.: 18/512,389

(22) Filed: Nov. 17, 2023

Related U.S. Application Data
(63) Continuation of application No. 16/621,792, filed on Dec. 12, 2019, now Pat. No. 11,879,123.
(60) Provisional application No. 62/523,225, filed on Jun. 21, 2017.
(60) Provisional application No. 62/523,202, filed on Jun. 21, 2017.
(60) Provisional application No. 62/552,829, filed on Aug. 31, 2017.
(60) Provisional application No. 62/552,785, filed on Aug. 31, 2017.
(60) Provisional application No. 62/614,213, filed on Jan. 5, 2018.
(60) Provisional application No. 62/624,299, filed on Jan. 31, 2018.

(51) Int. Cl. C12N 1/20; A61K 9/00; A61K 35/74; A61K 35/741; C07K 14/245; C12N 15/63; C12Q 1/527

(52) U.S. Cl. CPC C12N 1/20; A61K 9/0053; A61K 35/74; A61K 35/741; C07K 14/245; C12N 15/63; C12N 2510/00; C12N 2795/00021; C12N 2800/202; C12Q 1/527

(57) ABSTRACT

Modified probiotics, pharmaceutical compositions thereof, and methods of modulating and treating disorders are disclosed.

### RELATED APPLICATIONS

The instant application is a continuation application based upon U.S. patent application Ser. No. 16/621,792, filed Dec. 12, 2019, which is a national stage application under 35 U.S.C. § 371 of international application number PCT/US2018/038840, filed Jun. 21, 2018, which further claims priority to U.S. Provisional Application No. 62/523,225, filed Jun. 21, 2017; U.S. Provisional Application No. 62/552,785, filed Aug. 31, 2017; U.S. Provisional Application No. 62/552,829, filed Aug. 31, 2017; U.S. Provisional Application No. 62/614,213, filed Jan. 5, 2018; U.S. Provisional Application No. 62/624,299, filed Jan. 31, 2018, and U.S. Provisional Application No. 62/523,202, filed Jun. 21, 2017. The entire contents of each of the foregoing applications are hereby expressly incorporated herein by reference in their respective entireties.

### SEQUENCE LISTING

The application contains a Sequence Listing which has been submitted electronically in .XML format and is hereby incorporated by reference in its entirety. Said .XML copy, created on Nov. 6, 2023, is named “126046-09402.xml” and is 502,239 bytes in size. The sequence listing contained in this .XML file is part of the specification and is hereby incorporated by reference herein in its entirety.

### BACKGROUND

A growing body of scientific evidence suggests that probiotic bacteria are beneficial in the treatment or prevention of various diseases or disorders associated with the gut, including, for example, gastrointestinal disorders such as Crohn's disease and inflammatory bowel syndrome. More recently, genetically engineered bacteria have emerged as a potential new therapeutic treatment modality for gastrointestinal diseases and have also opened the field of bacterial therapies to a large number of other indications, including metabolic diseases, inflammatory diseases, and cancer. One benefit of genetically engineered bacteria is the ability to specifically target one or more disease mechanisms. For example, for gastrointestinal disorders, bacteria can be engineered to contain genes for the expression of anti-inflammatory agents or agents that aid in the healing of a disrupted gut-barrier, such as the short chain fatty acid butyrate, e.g., as described in International Patent Publication WO2016141108. Genetically engineered bacteria may also be considered as a treatment modality for various metabolic disorders, including but not limited to rare metabolic disorders arising from inborn errors in metabolism or IEMs. For example, as described in International Patent Publication WO2016090343, bacteria have been genetically modified to treat phenylketonuria (PKU) by expressing one or more enzymes which metabolize phenylalanine and thereby consuming excess phenylalanine within the gastrointestinal tract.

Bacteriophage are the most common biological entity in the world, and it is well documented that a majority of bacterial species, both gram positive and gram negative, contain one or more DNA bacteriophages which are integrated as so-called prophages in the bacterial chromosome (Clokie et al, Phages in Nature, Bacteriophage. 2011 January-February; 1(1): 31-45).

DNA phages can be lytic or temperate. Lytic phages infect bacterial cells and then program the synthesis of progeny phages, which are then released from the lysed cell. Conversely, temperate DNA phages establish a stable relationship with their host bacteria in which the integrated phage DNA, i.e., the prophage, is replicated in concert with the host's genome, and any host-damaging phage genes are not expressed. However, bacteriophage particles can be released from cells containing an intact prophage by a process called induction, during which prophage genes required for lytic growth are turned on and progeny phage particles are produced and released from the cell through lysis of the cell (reviewed in Casjens, Prophages and bacterial genomics: what have we learned so far?; Mol Microbiol. 2003 July; 49(2):277-300). In some cases, induction can occur spontaneously and randomly in a small or large fraction of the bacteria that harbor the prophage. In other cases, specific, often undefined, environmental signals can cause simultaneous induction of a particular prophage in many cells, causing death of the bacterial cells.

Not all prophages have the ability to undergo a lytic cycle. Non-functional, i.e., defective or cryptic prophages can accrue to a high level of abundancy in many bacteria as a result of mutational decay and/or the loss of one or more genes essential to the lytic cycle over thousands of bacterial replication cycles (Bobay et al., Pervasive domestication of defective prophages by bacteria, Proc Natl Acad Sci USA. 2014 Aug. 19; 111(33): 12127-12132, and references therein).

### SUMMARY

In some embodiments, the disclosure provides a bacterium comprising one or more phage genome(s), wherein one or more of the phage genomes are defective. In some embodiments, the disclosure provides a bacterium comprising one or more phage genome(s), wherein one or more of the phage genomes are defective such that lytic phage is not produced. In some embodiments, the disclosure provides a bacterium comprising one or more phage genome(s), wherein one or more of the phage genomes are defective in that one or more phage genes are not expressed. In some embodiments, the disclosure provides a bacterium comprising one or more phage genome(s), wherein one or more phage genes in the one or more phage genome(s) comprise one or more mutations. In some embodiments, the one or more phage genome(s) are present in the natural state of the probiotic bacterium. In some embodiments, the bacteria encode one or more lysogenic phage(s). In some embodiments, the bacteria encode one or more defective or cryptic phage(s) or satellite phage(s). In some embodiments, the bacteria encode one or more tailiocins or gene transfer agents.

In some of the embodiments of the disclosure, one or more of the phage genomes are mutated. Such mutations may include one or more deletion(s) of a part of or the complete sequence of one or more phage genes. Alternatively, the mutations may include one or more insertion(s) of one or more nucleotides into one or more phage genes. In another example, the mutations may include one or more substitution(s) of a part of or the complete sequence of one or more phage genes. In another example, the mutations include one or more inversion(s) of a part of or the complete sequence of one or more phage genes in the phage genome. Additionally, the mutations may include any combination of one or more deletions, insertions, substitutions or inversions. In certain embodiments, the one or more mutations reduce or prevent the production and release of phage particles from the bacterium relative to the same bacterium not having the one or more targeted mutations in the one or more phage genomes. In some embodiments, the bacterium is a probiotic bacterium. Non-limiting examples of such probiotic bacteria include Bacteroides, Bifidobacterium, Clostridium, Escherichia, Lactobacillus, and Lactococcus. In some embodiments, the bacterium is Escherichia coli strain Nissle. In some embodiments, the phage genome which is mutated is E. coli Nissle Phage 1 genome, the E. coli Nissle Phage 2 genome and/or the E. coli Nissle Phage 3 genome. In one embodiment, the mutated phage genome is the E. coli Nissle Phage 3 genome. In one embodiment, the mutations are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the mutations, e.g., one or more deletions, are located in or comprise one or more genes selected from ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. pharmaceutically acceptable composition comprising the bacterium disclosed herein and a pharmaceutically acceptable carrier.

In some embodiments, the bacteria further comprise one or more circuits for the expression of one or more effector molecules.

In some embodiments, the disclosure relates to compositions and therapeutic methods for reducing hyperphenylalaninemia. In some embodiments, the compositions comprise a genetically engineered bacterium that is capable of expressing a phenylalanine metabolizing enzyme (PME). See, e.g., WO2017087580 A1, the contents of which are herein incorporated by reference in entirety. Phenylalanine is an essential amino acid primarily found in dietary protein. Typically, a small amount is utilized for protein synthesis, and the remainder is hydroxylated to tyrosine in an enzymatic pathway that requires phenylalanine hydroxylase (PAH) and the cofactor tetrahydrobiopterin. Hyperphenylalaninemia is a group of diseases associated with excess levels of phenylalanine, which can be toxic and cause brain damage. Primary hyperphenylalaninemia is caused by deficiencies in PAH activity that result from mutations in the PAH gene and/or a block in cofactor metabolism.

PKU is a severe form of hyperphenylalaninemia caused by mutations in the PAH gene. PKU is an autosomal recessive genetic disease that ranks as the most common inborn error of metabolism worldwide (1 in 3,000 births), and affects approximately 13,000 patients in the United States. More than 400 different PAH gene mutations have been identified (Hoeks et al., 2009). A buildup of phenylalanine (phe) in the blood can cause profound damage to the central nervous system in children and adults. If untreated in newborns, PKU can cause irreversible brain damage. Treatment for PKU currently involves complete exclusion of phenylalanine from the diet. Most natural sources of protein contain phenylalanine which is an essential amino acid and necessary for growth. In patients with PKU, this means that they rely on medical foods and phe-free protein supplements together with amino acid supplements to provide just enough phenylalanine for growth. This diet is difficult for patients and has an impact on quality of life.

Current PKU therapies require substantially modified diets consisting of protein restriction. Treatment from birth generally reduces brain damage and mental retardation (Hoeks et al., 2009; Sarkissian et al., 1999). However, the protein-restricted diet must be carefully monitored, and essential amino acids as well as vitamins must be supplemented in the diet. Furthermore, access to low protein foods is a challenge as they are more costly than their higher protein, nonmodified counterparts (Vockley et al., 2014). In children with PKU, growth retardation is common on a low-phenylalanine diet (Dobbelaere et al., 2003). In adulthood, new problems such as osteoporosis, maternal PKU, and vitamin deficiencies may occur (Hoeks et al., 2009). Excess levels of phenylalanine in the blood, which can freely penetrate the blood-brain barrier, can also lead to neurological impairment, behavioral problems (e.g., irritability, fatigue), and/or physical symptoms (e.g., convulsions, skin rashes, musty body odor). International guidelines recommend lifelong dietary phenylalanine restriction, which is widely regarded as difficult and unrealistic (Sarkissian et al., 1999), and “continued efforts are needed to overcome the biggest challenge to living with PKU—lifelong adherence to the low-phe diet” (Macleod et al., 2010).

In a subset of patients with residual PAH activity, oral administration of the cofactor tetrahydrobiopterin (also referred to as THB, BH4, Kuvan, or sapropterin) may be used together with dietary restriction to lower blood phenylalanine levels. However, cofactor therapy is costly and only suitable for mild forms of phenylketonuria. The annual cost of Kuvan, for example, may be as much as $57,000 per patient. Additionally, the side effects of Kuvan can include gastritis and severe allergic reactions (e.g., wheezing, lightheadedness, nausea, flushing of the skin).

The enzyme phenylalanine ammonia lyase (PAL) is capable of metabolizing phenylalanine to non-toxic levels of ammonia and transcinnamic acid. Unlike PAH, PAL does not require THB cofactor activity in order to metabolize phenylalanine. Studies of oral enzyme therapy using PAL have been conducted, but “human and even the animal studies were not continued because PAL was not available in sufficient amounts at reasonable cost” (Sarkissian et al., 1999). A pegylated form of recombinant PAL (PEG-PAL) is also in development as an injectable form of treatment. However, most subjects dosed with PEG-PAL have suffered from injection site reactions and/or developed antibodies to this therapeutic enzyme (Longo et al., 2014). Thus, there is significant unmet need for effective, reliable, and/or long-term treatment for diseases associated with hyperphenylalaninemia, including PKU. There is an unmet need for a treatment that will control blood Phe levels in patients while allowing consumption of more natural protein.

In some embodiments, the disclosure provides genetically engineered bacteria that encode and express phenylalanine ammonia lyase and/or phenylalanine hydroxylase and/or L-aminoacid deaminase and are capable of reducing hyperphenylalaninemia. The enzyme phenylalanine ammonia lyase (PAL) is capable of metabolizing phenylalanine to non-toxic levels of ammonia and transcinnamic acid. Unlike PAH, PAL does not require THB cofactor activity in order to metabolize phenylalanine. L-amino acid deaminase (LAAD) catalyzes oxidative deamination of phenylalanine to generate phenylpyruvate, and trace amounts of ammonia and hydrogen peroxide. Phenylpyruvic acid (PPA) is widely used in the pharmaceutical, food, and chemical industries, and PPA is the starting material for the synthesis of D-phenylalanine, a raw intermediate in the production of many chiral drugs and food additives. LAAD has therefore been studied in the context of industrial PPA production (Hou et al. 2015, Appl Microbiol Biotechnol. 2015 October; 99(20):8391-402; “Production of phenylpyruvic acid from L-phenylalanine using an L-amino acid deaminase from Proteus mirabilis: comparison of enzymatic and whole-cell biotransformation approaches”). Phenylpyruvate is unable to cross the blood brain barrier (Steele, Fed Proc. 1986 June; 45(7):2060-4; “Blood-brain barrier transport of the alpha-keto acid analogs of amino acids.” indicating that this conversion is useful in controlling the neurological phenotypes of PKU.

In certain aspects, the disclosure relates to genetically engineered bacteria that are capable of reducing hyperphenylalaninemia in a mammal. In certain aspects, the compositions and methods disclosed herein may be used for treating diseases associated with hyperphenylalaninemia, e.g., phenylketonuria. In certain embodiments, the genetically engineered bacteria are non-pathogenic and may be introduced into the gut in order to reduce toxic levels of phenylalanine. In certain embodiments, the phenylalanine ammonia lyase and/or phenylalanine hydroxylase and/or L-aminoacid deaminase is stably produced by the genetically engineered bacteria, and/or the genetically engineered bacteria are stably maintained in vivo and/or in vitro. In certain embodiments, the genetically engineered bacteria further comprise a phenylalanine transporter gene to increase their uptake of phenylalanine. The invention also provides pharmaceutical compositions comprising the genetically engineered bacteria, and methods of modulating and treating disorders associated with hyperphenylalaninemia.

The engineered bacteria may also contain one or more gene sequences relating to bio-safety and/or bio-containment, e.g., a kill-switch, gene guard system, and/or auxotrophy. In some embodiments, the engineered bacteria may contain an antibiotic resistance gene. The expression of any these gene sequence(s) may be regulated using a variety of promoter systems, such as any of the promoter systems disclosed herein, which promoter system may involve use of the same promoter to regulate one or more different genes, may involve use of a different copy of the same promoter to regulate different genes, and/or may involve the use of different promoters used in combination to regulate the expression of different genes. The use of different regulatory or promoter systems to control gene expression provides flexibility (e.g., the ability to differentially control gene expression under different environmental conditions and/or the ability to differentially control gene expression temporally) and also provides the ability to “fine-tune” gene expression, any or all of which regulation may serve to optimize gene expression and/or growth of the bacteria.

In some embodiments, the bacteria are capable of expressing any one or more effector molecules in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut or the tumor microenvironment, such as arabinose. In some embodiments, any one or more of the circuits are present on one or more plasmids (e.g., high copy or low copy) or are integrated into one or more sites in the bacterial chromosome. Also, in some embodiments, the genetically engineered bacteria further comprise one or more of the following: (1) one or more auxotrophies, such as any auxotrophies known in the art and provided herein, e.g., thyA or dapB auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits. Compositions of the bacteria and methods for the treatment, prevention, or management of one or more diseases or disorders are also provided.

### BRIEF DESCRIPTION OF THE FIGURES

FIG. 1A depicts a schematic of locations of predicted phage on the EcN Genome (CP007799.1) from PHAST Analysis. The three high-scoring, intact phages are labeled as Phages 1-3. Phage 1 (PHAST score 110) is 18.8 kb long and stretches from coordinates 241,563-260,441 within the EcN genome. Phage 2 (PHAST score 150) is 52.4 kb long and stretches from 1,325,883-1,378,287. Phage 3 (PHAST score 150) is 59 kb long and stretches from 3,405,101-3,418,180. Also identified were several low-scoring phage, designated as “incomplete” or “questionable” by the PHAST algorithm, which do not contain all the major components of a phage and could therefore represent partial phage, or false positive predictions. Abbreviations: EcN=Escherichia coli Nissle 1917; PHAST=Phage Search Tool software; kb=kilobases. FIG. 1B depicts a table describing 5 putative prophage in the Nissle genome (3 intact, 1 incomplete, and 1 questionable) according to PHASTER scoring.

FIG. 2 depicts a schematic showing 1 of 3 high-scoring phage in Nissle using the Phast tool, referred to herein as “Phage 1”, and which contains all major components of a phage. Putative genes are labeled Hyp=Hypothetical, PLP=other phage like protein, Oth=Other, RNA=tRNA, TRA=transposase.

FIG. 3 depicts a schematic showing 1 of 3 high-scoring phage in Nissle using the Phast tool, referred to herein as “Phage 2”, and which contains all major components of a phage. Putative genes are labeled Hyp=Hypothetical, PLP=other phage like protein, Oth=Other, RNA=tRNA, TRA=transposase, Lys=Lysis, Ter=Terminase, Coa=Coat, Sha=Tail shaft, Fib=Tail fiber.

FIG. 4 depicts a schematic showing 1 of 3 high-scoring phage in Nissle using the Phast tool, referred to herein as “Phage 3”, and which contains all major components of a phage. Putative genes are labeled Hyp=Hypothetical, PLP=other phage like protein, Oth=Other, RNA=tRNA, TRA=transposase, Lys=Lysis, Ter=Terminase, Coa=Coat, Sha=Tail shaft, Fib=Tail fiber.

FIG. 5A depicts the first of 2 lower scoring “incomplete” or “questionable” phage identified using the Phast tool. Putative genes are labeled Hyp=Hypothetical, PLP=other phage like protein, Oth=Other, RNA=tRNA, TRA=transposase, int=Integrase, Att=attachment site.

FIG. 5B depicts the second of 2 lower scoring “incomplete” or “questionable” phage identified using the Phast tool. Putative genes are labeled Hyp=Hypothetical, PLP=other phage like protein, Oth=Other.

FIG. 6 depicts a schematic of the predicted Nissle Phage 3 sequence (59,056 bp).

FIG. 7 depicts a schematic of the Phage 3 deletion within the Phage 3 genome used in SYN-PKU-2002.

FIG. 8 depicts a schematic showing a 49,496 bp Phage 3 sequence comprising a knockout deletion, e.g., as comprised in SYN-PKU-2002.

FIG. 9 depicts a schematic of the section of the phage that can be deleted to inactivate the phage, e.g., as deleted in SYN-PKU-2002.

FIG. 10 depicts a schematic showing partial regions within the 43 kb Phage 3 sequence that align to sequence in other E. coli strains. The sequence identified as Phage 3 was compared against 5691 E. coli and Shigella genome assemblies downloaded from NCBI. Listed in the column on the left are the accession numbers of the E. coli genomes that were positive in the analysis. Across the top of the Figure are the coordinates of the DNA sequence according to the number of kb from the start of the sequence. The lines depict the sequence from each specific E. coli genome that align with the DNA sequence of Phage 3. Abbreviations: E. coli=Escherichia coli; kb=kilobase; NCBI=National Center for Biotechnology Information; DNA=deoxyribonucleic acid.

FIG. 11 depicts a schematic showing partial regions within the 43 kb Phage 3 sequence that align to sequence in other E. coli strains. The area in the shaded box was chosen as a site for deletion in the Phage 3 knockout strategy.

FIG. 12 depicts a bar graph showing the distribution of the number of predicted “intact” phage across a set of 287 E. coli genomes from Refseq. The Refseq database was analyzed for the number of intact phage that were present in published, complete E. coli genomes. The histogram displays a non-normal distribution, but it is clear that nearly all E. coli genomes contain intact prophage, and the majority of published, complete E. coli genomes contain more intact prophage than EcN. Abbreviations: E. coli=Escherichia coli; EcN=Escherichia coli Nissle 1917; Refseq=reference sequence.

FIG. 13 depicts an DNA gel electrophoresis study verifying phage-specific PCR Primers. The performance of PCR primer pairs for Phages 1, 2 and 3 and rpoB against EcN genomic DNA is shown. Abbreviations: bp=base pair; EcN=E. coli Nissle; rpoB=3 subunit of bacterial RNA polymerase.

FIG. 14 depicts DNA gel electrophoresis study showing EcN Prophage Regions Amplified from ATCC 13706 Plaque Plugs. Abbreviations: bp=base pairs; EcN=Escherichia coli Nissle 1917; rpoB=3 subunit of bacterial RNA polymerase.

FIG. 15 depicts blood phenylalanine concentrations relative to baseline at 4 hours post SC phenylalanine injection, comparing strains SYN-PKU710 and SYN-PKU708. Mice were administered single dose of phenylalanine by subcutaneous injection at 0.1 mg per gram body weight. At 1, 2 and 3 h post Phe challenge, the bacteria (or water) were administered to mice by oral gavage (300 ul/dose, total of 3×e10 cfu/mouse). The percentage decrease in deltaPhe SYN-PKU710 and SYN-PKU708 were calculated to be 29% and 40%, respectively.

FIG. 16A and FIG. 16B depict line graphs showing TCA production (FIG. 16A) and Phenylpyruvate production (FIG. 16B) in SYN-PKU-2002. Both are measures of the degradation of phenylalanine in vitro by SYN-PKU-2002. SYN-PKU-2002 was prepared by growth in Lysogeny Broth (LB) either aerobically (the uninduced state) or anaerobically with the addition of IPTG and arabinose (the induced state). In vitro, incubation of activated SYN-PKU-2002 in the presence of phenylalanine results in the production of TCA and PP over time, demonstrating that SYN-PKU-2002 is capable of metabolizing phenylalanine.

FIG. 17A and FIG. 17B depict the change in phenylalanine levels and hippurate recovery in mice gavaged with either streptomycin resistant Nissle or phage free strain SYN-PKU-2002 (which is phage free SYN-PKU-710). Mice were administered a single dose of phenylalanine (0.1 mg per gram body weight) by subcutaneous injection. At 1, 2 and 3 h post Phe challenge, the bacteria (or water no shown) were administered to mice by oral gavage (3×250 ul). Whole blood was collected via submandibular bleed at each time point and analyzed for phenylalanine levels. Urine collection in metabolic caging commenced immediately after the 1^{st} bacterial dose and continued to be collected for the duration of the study and analyzed for hippurate levels.

FIG. 18A and FIG. 18B depicts a graph showing changes in phenylalanine levels post Phe challenge (FIG. 18A) and hippurate recovery (FIG. 18B) from urine collected from animals treated with the indicated doses of SYN-PKU-2002. In brief, animals were transferred to metabolic cages (3 mice per cage, 2 cages per group) and administered single dose of phenylalanine by subcutaneous injection (0.1 mg per gram body weight). At 1, 2 and 3 h post Phe challenge, bacteria were administered to mice by oral gavage at the doses 1×10¹¹, 5×10¹⁰, 2.5×10¹⁰, 1.25×10¹⁰, 6.25×10⁹, or 3.13×10⁹ cells. SYN-PKU901 was gavaged to a control group (n=9) at the highest dose of 1×10¹¹ cells. Urine was collected from all animals up to 4 h post Phe challenge. Blood was obtained by submandibular bleed at T=0 h and at T=4 h at the highest dose group (1×10¹¹ cells) for both SYN-PKU-2002 and SYN-PKU901-treated mice for the determination of changes in serum Phe.

FIG. 19 depicts a graph showing the outcome of an in vivo competition study between phage containing and phage free strains SYN-PKU-713 and SYN-PKU-2001. Mice were administered equal amounts (approx. 3×10\hat{ }9 of cells) daily for three days. Each day fecal pellets were collected and CFUs determined in plating assay based on the different antibiotic resistances of the two strains, as described in the Examples. Results indicate that there is no large difference in transit or colonization between the phage-free PKU strain of Nissle SYN-PKU-713 and SYN-PKU-2001.

FIG. 20 depicts a graph showing measurements of gastric phenylpyruvate in two pigs at various times prior and post administration of SYN-PKU-2001.

FIG. 21 depicts a graph showing conversion efficiency of oral trans-cinnamate to urinary hippurate in non-human primates. NHPs (n=6) were orally administered ¹³C-trans-cinnamate (¹³C-TCA) and urine was collected over 6 h. ¹³C-Hippurate (¹³C-HA) was measured by mass spectroscopy. The percentage of urinary ¹³C-HA recovered as a function of ¹³C-TCA administered was calculated and used as a normalization factor for HA recovery in subsequent experiments. This factor accounts for TCA that is not converted to HA or that is lost to incomplete urinary collection, thus allowing a more accurate description of strain activity.

FIGS. 22A, 22B, 22C, 22D, and 22E depict graphs showing profiling and efficacy in non-human primates (NHPs). In FIG. 22A, fasted NHPs (n=6) were administered a 5 g peptide (left) or mock challenge (right) alone (black bars) or with 5×10¹¹ cells of SYN-PKU-2002 (striped bars) and urine was collected for 6 h. Normalized HA recovery is shown as the average±standard deviation. Animals receiving SYN-PKU during the studies performed in FIG. 22A were also administered a dose of ¹³C-phenylalanine intravenously (IV) 1 h after peptide or mock challenge (FIG. 22B). Normalized urinary ¹³C-HA, which could only be derived from the IV administered ¹³C-Phe, was found in animals that received a peptide challenge and is displayed as black bars. No urinary ¹³C-HA was recovered in animals that remained fasting. In FIG. 22C, fasted NHPs were administered an oral dose of d₅-phenylalanine (d₅-Phe) with or without administration of SYN-PKU-2002. The dashed line represents the quantity of d₅-Phe administered. d₅-hippurate (d₅-HA) was only found in animals that received SYN-PKU-2002 (striped bar). Data is representative of the average normalized d₅-HA recovery±standard deviation (n=6). Serum d₅-Phe was measured in NHPs that received SYN-PKU-2002 (light grey line) or mock administration (dark gray line) (FIG. 22D). Data represent the average d₅-Phe concentration±standard deviation (n=6) In FIG. 22E, NHPs received a d₅-Phe alone or with 5×10¹¹ cells of SYN-PKU-2002. Blood was collected over 6 h and areas under the curve for serum d₅-Phe were calculated. Data show AUCs plus and minus the upper and lower bounds of the 90% credible level respectively.

FIG. 23A and FIG. 23B depict graphs showing SYN-PKU-2002 specific metabolite detection in serum of non-human primates. Using LC-MS/MS, serum concentrations of d5-HA (FIG. 23A) and d5-TCA (FIG. 23B) were determined in non-human primates administered d5-Phe and SYN-PKU-2002 orally. No detectable d5-HA or d5-TCA was detected when d5-Phe was administered in the absence of SYN-PKU-2002 (data not shown). The presence of these metabolites demonstrates SYN-PKU-2002-specific activity in these animals.

FIG. 24A and FIG. 24B depict the conversion of trans-cinnamate to urinary hippurate in NHPs.

FIG. 25 depicts a graph showing that SYN-PKU-2002 metabolizes Phe when administered orally in healthy non-human primates (NHPs). Gavage with SYN-PKU-2002 reduces the spike in blood phe levels observed upon administration of protein challenge together with radio-labelled Phe.

FIG. 26A, FIG. 26B, FIG. 26C, and FIG. 26D depict graphs showing SYN-PKU-2002 dose-dependent conversion of Phe and production of plasma biomarkers in non human primates upon single dose of SYN-PKU-2002 with protein meal, illustrating significant activity and efficacy of of SYN-PKU-2002 in the NHP model. Fasted NHPs (n=5 per dose group) were administered a 5 g peptide bolus with the indicated dose (CFUs) of SYN-PKU-2002. Urine was collected over 6 h and serum at 0, 0.5, 1, 2, 4, and 6 h. FIG. 26A depicts a graph showing normalized urinary HA recovery from dose groups shown as the average±standard deviation. FIG. 26B and FIG. 26C depict graphs showing the calculated AUCs for the concentrations of serum HA and. White bars represent the average AUC±standard deviation. FIG. 26D depicts a graph showing serum Phe concentration as determined at the indicated time points. The 3 highest doses administered in the dose response are shown compared to the No Cells control, as these 3 doses showed a significant reduction in serum Phe AUC (p<0.05).

FIG. 27A, FIG. 27B, and FIG. 27C depicts graphs showing SYN-PKU-2002 dose dependent conversion of Phe from casein (FIG. 27A) TCA levels (FIG. 27B), and hippuric acid (FIG. 27C) in NHP's. Blood metabolites were collected for 6 hours.

FIG. 28 depicts a graph showing SYN-PKU-2002 conversion of Phe in an NHP resulting in an increase in protein intake, which would correspond to a 2.5 fold increase in protein intake in a PKU patient.

FIG. 29 depicts a graph showing in vitro activity of SYN-PKU-2002. 1×10⁸ activated cells were analyzed in 50 mM Phe assay buffer for PAL (dark blue bars, left y-axis) and LAAD (light blue bars, right y-axis) activity. Cells were pre-induced with L-arabinose (+ara), IPTG (+IPTG) or in an anaerobic chamber (—O₂) and rates of TCA and PP were calculated by linear regression of TCA and PP production over time. The graph displays the average and standard deviation of three biological replicates.

FIG. 30 depicts a graph showing the effect of dapA deletion on SYN-PKU-2002 growth in vitro. To characterize the growth of E. coli Nissle (EcN) and SYN-PKU-2002, which contains a mutation in the dapA gene, both strains were incubated in LB that did (+) or did not (−) contain diaminopimelic acid (DAP; 100 μg/mL) at 37° C. for 960 minutes under constant shaking. The OD600 was measured every 10 minutes to assess cell growth over time. The average of three biological replicates and two technical replicates is plotted for each time point. Data shows that SYN-PKU-2002 is unable to grow without the addition of exogenous DAP to the growth media.

FIG. 31A and FIG. 31B depict graphs showing PAL activity of SYN-PKU-2002 against peptides. SYN-PKU-2002 was grown in a bioreactor and induced for PAL and LAAD activity. Activated cells were incubated for 60 min at 37° C. in Phe assay media containing 50 mM Phe in the form of free Phe, Phe-Pro, Phe-Gly-Gly, Phe-Val, Gly-Phe, or in 5 g/L peptone, or tryptone. The total concentration of trans-cinnamate (FIG. 31A) or phenylpyruvate (FIG. 31B) produced was determined by LC-MS/MS over time, and rates of TCA and PP production were calculated by linear regression. Significant PAL activity was observed with all substrates. LAAD activity was observed only when free Phe was used as a substrate, with a lesser amount of activity when complex substrates peptone or tryptone were used. The graph displays the average and standard deviation of three biological replicates.

FIG. 32 depicts a graph showing the growth characteristics of SYN766, a diaminopimelate auxotroph in various concentrations of diaminopimelate. SYN766 (E. coli Nissle 1917, ΔdapA) was incubated in growth media which contained decreasing concentrations of DAP at 37° C. for 960 minutes under constant shaking. The OD600 was measured every 10 minutes in order to assess cell growth over time. The average of three biological replicates is plotted for each time point.

FIG. 33 depicts a graph showing the growth characteristics of various strains of E. coli Nissle in LB growth media without diaminopimelate. To characterize the growth characteristics of various modified strains of EcN in absence of DAP, cultures were incubated in LB that did not contain DAP at 37° C. for 960 minutes under constant shaking. The OD600 was measured every 10 minutes to assess cell growth over time. The average of three biological replicates and two technical replicates is plotted for each time point.

FIG. 34 depicts a graph showing the growth characteristics of various strains of EcN in LB growth media with 100 μg/mL diaminopimelate. To characterize the growth characteristics of various modified strains of EcN in the presence of DAP, they were incubated in growth media with 100 μg/mL DAP, at 37° C. for 960 minutes, constantly shaking. The OD600 was measured every 10 minutes to assess cell growth over time. The average of three biological replicates is plotted for each time point.

FIGS. 35A, 35B, and 35C depict the effect of DAP auxotrophy and Phe degradation activity on EcN survival and transit in C57BL/6 Mice. FIG. 35A shows the effect of DAP auxotrophy on fecal clearance in group 1 mice (SYN-PKU901/SYN766). FIG. 35B depicts the effect of genetic engineering for Phe-degradation on fecal clearance in group 2 mice (SYN-PKU901/SYN3282). FIG. 35C depicts the effect of genetic engineering for Phe-degradation and DAP auxotrophy on fecal clearance in group 3 mice (SYN-PKU-2001/SYN3282). Mixed doses of bacteria were administered orally to C57BL/6 mice (n=5). Doses were plated for CFU counts in quadruplicate to determine the number of bacteria administered. At each time point, feces were collected, homogenized, and plated for bacterial CFU determination on antibiotic selective media. For each time point, data represent the average CFU/mg counts of 5 fecal samples±standard deviation, normalized for each strain as a fraction of the initial CFU dosed unless otherwise denoted in the appendix. This normalization allows direct comparison of survival/clearance between the 2 strains within a group, even with variation in the actual CFU of each strain administered at T=0.

FIGS. 36A-36F depicts the effect of DAP auxotrophy and Phe degradation activity on EcN transit and clearance in C57BL/6 mice. SYN-PKU901 or SYN-PKU-2001 were orally administered to C57BL/6 mice (9×109 CFU/dose, n=3/time point). At the indicated times, effluents from the stomach (A), upper small intestine (B), middle small intestine (C), lower small intestine (D), cecum (E) and colon (F) were collected and plated for CFU counts. CFU determination was performed by microdilution on antibiotic selective media. For each time point, data represent the CFUs determined from 3 effluent samples±standard deviation. No CFUs were determined in any sample at 48 h post-dose, indicating complete bacterial clearance.

FIG. 37 depicts a schematic of the SYN-PKU-2002 genome. The locations of the genomic modification sites in SYN-PKU-2002 are shown, with kbp designation indicating the chromosomal position relative to the 0/5.4 Mb reference marker. The chromosomal origin of replication is shown as a red line. Green text boxes designate PheP gene insertions, purple text boxes designate PAL gene insertions, orange text box designates LAAD gene insertion, and grey text boxes with Δ symbol designates the location of the dapA and Φ deletion. Italicized gene names in parenthesis refer to the upstream and downstream genes surrounding the inserted genes.

FIG. 38 depicts SYN-PKU-2002 dose-dependent conversion of Phe and production of plasma biomarkers in non-human primates upon single dose of SYN-PKU-2002 with protein meal, illustrating significant activity and efficacy of SYN-PKU-2002 in the NHP model. Fasted NHPs (n=5 per dose group) were administered a 5 g peptide bolus with the indicated dose (CFUs) of SYN-PKU-2002. Plasma was collected at 0, 0.5, 1, 2, 4, and 6 h after dosing at time 0. Each point represents the HA concentration measured in plasma at the time point after dosing. Standard deviations are shown as vertical bars at each point.

FIG. 39 depicts SYN-PKU-2001 characterization within the gastrointestinal tract of Non-Human Primates. Cynomolgus monkeys were dosed with 5.5 grams of peptone, 5 mL of 0.36 M sodium bicarbonate, 25 mg/kg of D5-Phenylalanine, and SYN-PKU-2001 and euthanized either 0.5 hours or 2 hours after dosing. Following euthanization, tissue samples were collected from various sections of the gastrointestinal tract and analyzed to determine the concentration of Phe and SYN-PKU-2001 in each section.

### DESCRIPTION OF EMBODIMENTS

In one aspect, the disclosure provides bacteria which contain an endogenous phage and comprise one or more modifications to the phage sequence. In some embodiments, the modifications alter the properties of the prophage sequence. Such mutations include one or more partial or complete deletion(s) of one or more phage genes, one or more insertion(s) of one or more nucleotides into one or more phage genes, one or more partial or complete substitution(s) of one or more phage genes in the phage genome; one or more inversion(s) of one or more phage genes or combinations thereof.

This disclosure provides compositions comprising novel bacteria for the treatment of a disorder, which comprise one or more bacteriophages or prophages in their natural state. In some embodiments, the bacteria comprise one or more modifications to the genomes of the one or more phages. In some embodiments, the one or more modifications render the phage or prophage inactive. In some embodiments, these bacteria are further genetically modified to comprise one or more genes for the expression or production of one or more effector molecules. Methods for the production and use of these genetically engineered bacteria in novel therapies for the treatment of disorders are provided.

In one embodiment, E. coli Nissle is used as a starting point, parental strain or “chassis” for the genetically engineered bacteria. In one embodiment, the bacteriophage which is modified is a phage which is endogenous to E. coli Nissle in its phage is present in the bacteria in their natural state.

In some embodiments, the genetically engineered bacteria comprise one or more genes encoding one or more effectors, e.g., PME(s). In some embodiments, the genetically engineered bacteria comprise one or more genes encoding PAL. In some embodiments, the genetically engineered bacteria comprise one or more genes encoding LAAD. In some embodiments, the genetically engineered bacteria comprise one or more genes encoding PAL and one or more genes encoding LAAD. In some embodiments, the genetically engineered bacteria comprise one or more genes encoding a transporter, e.g., PheP. In some embodiments, the genetically engineered bacteria comprise one or more genes encoding a transporter, e.g., PheP and one or more genes encoding PAL. In some embodiments, the genetically engineered bacteria comprise one or more genes encoding a transporter, e.g., PheP and one or more genes encoding LAAD. In some embodiments, the genetically engineered bacteria comprise one or more genes encoding a transporter, e.g., PheP, one or more genes encoding LAAD, and one or more genes encoding PAL. In any of the preceding embodiments, the genetically engineered bacteria for the consumption of phenylalanine further comprise one or more relative to its original state. In some embodiments, the endogenous bacteriophage genomes. In some embodiments, the bacteriophage(s) have been mutated in one or more genes within the bacteriophage genome. Such mutations include deletions, insertions, substitutions and inversions and are located in or encompass one or more bacteriophage genes.

Bacteriophage are the most common biological entity in the world, and it is well documented that a majority of bacterial species, both gram positive and gram negative, contain one or more DNA bacteriophages which are integrated as so-called prophages in the bacterial chromosome (Clokie et al, Phages in Nature, Bacteriophage. 2011 January-February; 1(1): 31-45). For example, two separate studies on E. coli strains studies showed that 51 different functional phages were released from 27 E. coli strains analyzed, and 83 of 107 E. coli strains tested released at least one functional phage type (Casjens, Prophages and bacterial genomics: what have we learned so far?; Mol Microbiol. 2003 July; 49(2):277-300; Osawa et al., Genotypic variations of Shiga toxin-converting phages from enterohaemorrhagic Escherichia coli O157:H7 isolates; J Med Microbiol (2001) 49: 565-574, and Schicklmaier et al., A comparative study on the frequency of prophages among natural isolates of Salmonella and Escherichia coli with emphasis on generalized transducers. Antonie Van Leeuwenhoek (1998) 73: 49-54).

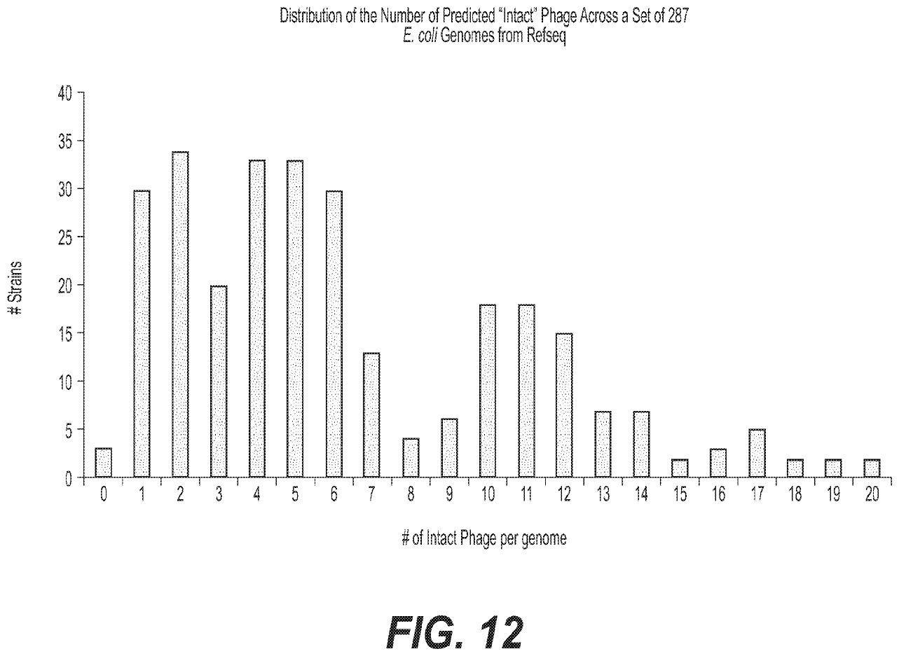

As shown in FIG. 12, nearly all E. coli genomes contain intact prophage, and the majority of published, complete E. coli genomes contain more intact prophage than EcN. Abbreviations: E. coli=Escherichia coli; EcN=Escherichia coli Nissle 1917; Refseq=reference sequence.

Among Gram-positive bacteria, the genomes of B. subtilis, Clostridium acetobutylicum, Lactococcus lactis, and many others have been shown to include largely intact prophages (Kunst et al., 1997; Bolotin et al., The complete genome sequence of the gram-positive bacterium Bacillus subtilis. Nature (2001) 390: 249-256, Nolling et al., Genome sequence and comparative analysis of the solvent-producing bacterium Clostridium acetobutylicum. J Bacteriol (2001) 183: 4823-4838; Bolotin et al., The complete genome sequence of the lactic acid bacterium Lactococcus lactis ssp. lactis IL1403. Genome Res (2001) 11: 731-753).

DNA phages can be lytic or temperate. Lytic phages infect bacterial cells and then program the synthesis of progeny phages, which are then released from the lysed cell. Conversely, temperate DNA phages establish a stable relationship with their host bacteria in which the integrated phage DNA, i.e., the prophage, is replicated in concert with the host's genome, and any host-damaging phage genes are not expressed. However, bacteriophage particles can be released from cells containing an intact prophage by a process called induction, during which prophage genes required for lytic growth are turned on and progeny phage particles are produced and released from the cell through lysis of the cell (reviewed in Casjens, Prophages and bacterial genomics: what have we learned so far?; Mol Microbiol. 2003 July; 49(2):277-300). Induction can occur in some cases spontaneously and randomly in a small or large fraction of the bacteria that harbor the prophage, or specific, often undefined, environmental signals can cause simultaneous induction of a particular prophage in many cells, causing death of the bacterial cells. In some cases, presence of prophage sequences may also allow some bacteria to have properties they would not have without the phage, such as antibiotic resistance, the ability to exist in different environmental conditions, improved adhesion, pathogenicity or facilitated horizontal gene transfer (Casjens et al., 2001).

Not all prophage have the ability to undergo a lytic cycle. Non-functional, i.e., defective or cryptic prophages can accrue to a high level of abundancy in many bacteria as a result of mutational decay and/or the loss of one or more genes essential to the lytic cycle over thousands of bacterial replication cycles (Bobay et al., Pervasive domestication of defective prophages by bacteria, Proc Natl Acad Sci USA. 2014 Aug. 19; 111(33): 12127-12132, and references therein). Of note, defective prophages often also contain a number of genes that can provide advantageous functionality to the host, including genes encoding proteins with homologous recombination functions, prevention of further infection, or bacteriocins, which may be helpful in competition for nutrients, e.g., through growth inhibition of other neighboring bacterial species.

Phages can positively affect gene expression and fitness in E. coli in numerous ways. Cryptic, lysogenic, and lytic phages have been shown to provide multiple benefits to the host promoting survival in adverse environmental conditions. For example, gene sequences transferred to the bacterium by phages have been linked to adaptation to different nutrients or a different niche, or to increased ability to eliminate competing strains. Dormant prophage has also been shown to prevent superinfection with another, e.g., lytic, phage.

Several studies have shown that endogenous phages affect the ability of bacteria to grow in certain carbon sources. Along with lambda, active Mu, P1 and P2 prophages and cryptic prophage CP4-57 increase growth under glucose-limited and other growing conditions (Edlin, G., Lin, L. & Bitner, R. Reproductive fitness of P1, P2, and Mu lysogens of Escherichia coli. J. Virol. 21, 560-564 (1977); Edlin, G., Lin, L. & Kudmar, R. λ Lysogens of E. coli reproduce more rapidly than non-lysogens. Nature 255, 735-737 (1975); Wang, X., Kim, Y. & Wood, T. K. Control and benefits of CP4-57 prophage excision in Escherichia coli biofilms. ISME J. 3, 1164-1179 (2009). In another study, it was shown that when λ integrates into the E. coli genome, ability of the cell to grow on poor carbon sources is shut down. IN this case, limitation of metabolism may confer a survival benefit to the bacterium. Slowing bacterial growth in glucose-poor environments might help the bacterium, elude detection by the immune system, increasing the chances of survival.

Other survival properties may be affected as well. Wang et al created a single E. coli strain lacks all nine cryptic prophages. In this study, it was shown that these prophages are beneficial for withstanding osmotic, oxidative and acid stresses, for increasing growth under various conditions, enhancing phosphorus and nitrogen utilization, and for influencing biofilm formation (Wang et al., Cryptic prophages help bacteria cope with adverse environments; DOI: 10.1038/ncomms1146). In pathogenic bacteria prophage, several studies suggest that acquisition is associated with changes in pathogen virulence.

Accordingly, a skilled artisan might expect that modification, e.g., mutation or deletion of portions or entirety of an endogenous prophage may alter, e.g., negatively affect, bacterial fitness. Additionally, one might assume that endogenous prophage may alter, e.g., negatively affect, effector activity in a genetically engineered bacterium capable of producing this effector. This may be especially the case if the endogenous prophage is present in all specimen of a particular strain subtype—this would indicate that the bacterium comprising the prophage sequences evolutionarily was able to out compete a form of the bacterium that lacks the prophage.

As described further in this disclosure, a prophage in E. coli Nissle was identified, which is capable of undergoing lysis under certain conditions, and which is present in all specimens of E. coli Nissle. Surprisingly, testing of bacterial fitness, residence time, and activity showed that the bacterium comprising the mutation or deletion in the endogenous phage was essentially the same, e.g., at least the same order of magnitude.

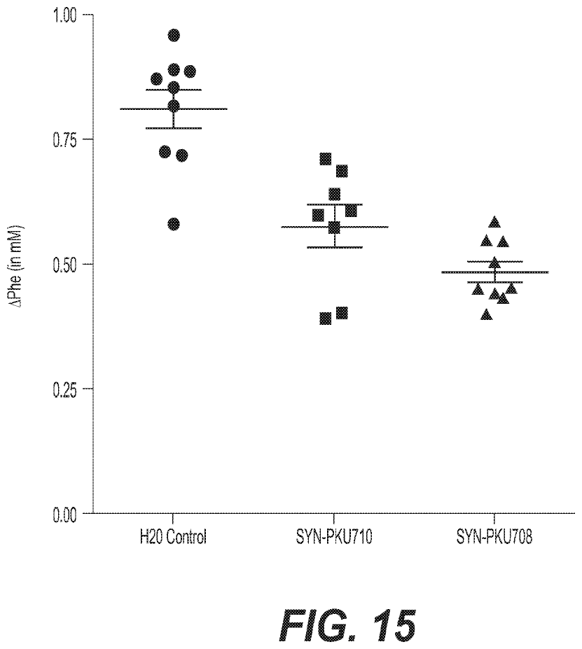

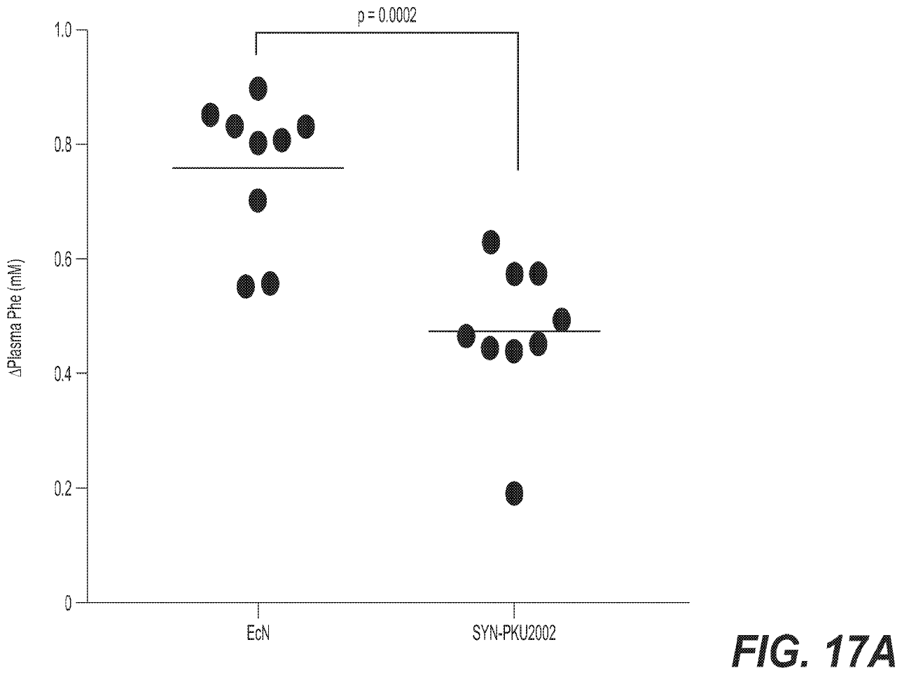

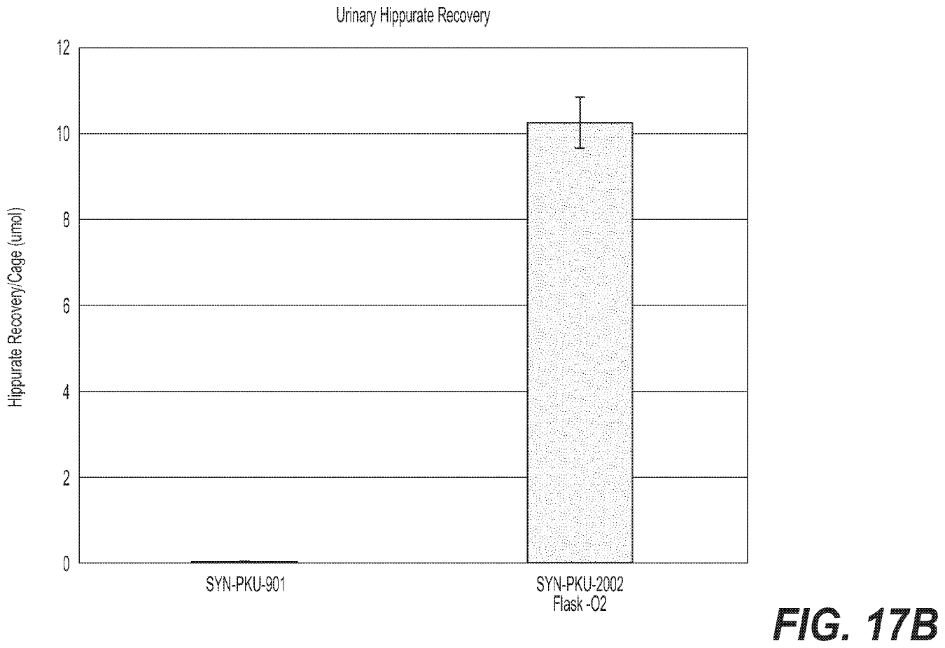

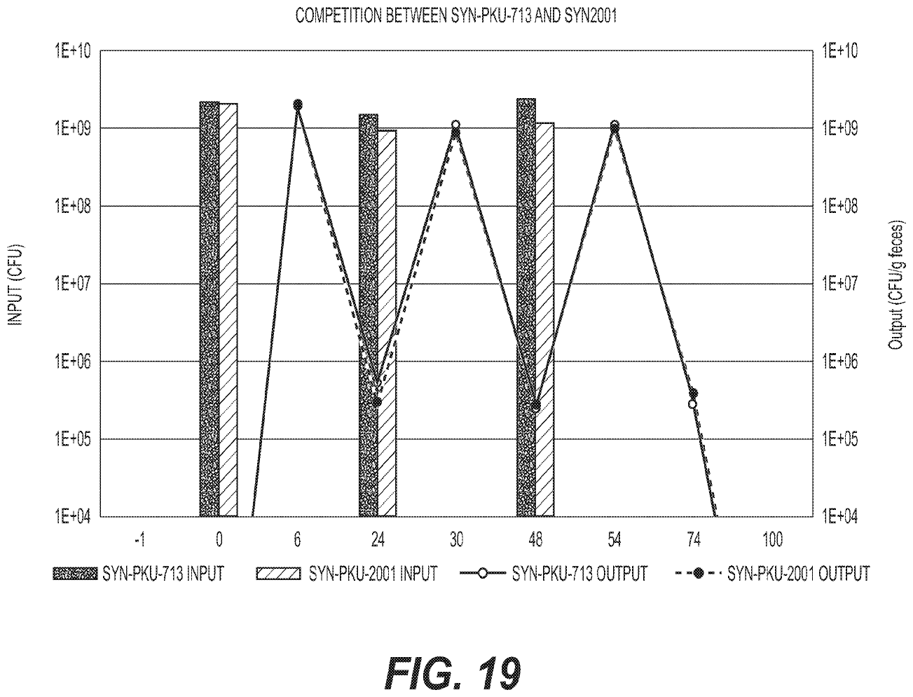

Under similar assay conditions, there was no discernable difference in Phe degradation activity (in vitro or in vivo) between the strains. For example, under similar assay conditions, Phe consumption is within the same magnitude between the two strains (see, e.g., FIG. 15 and FIG. 17A). In vivo competition studies between phage containing and phage free strains indicate that there is no discernable difference in transit or colonization between the phage-free PKU strain of Nissle (see, e.g. FIG. 19).

Accordingly, in some embodiments, one or more modification(s), e.g., mutation(s) or deletion(s) or other modifications described herein, in the genome of a phage does not alter the bacterial fitness of the modified or genetically engineered bacterium. In some embodiments, the engineered bacteria comprising one or more phage modifications, e.g., mutation(s) or deletion(s) or other modifications described herein, have essentially the same or at least similar bacterial fitness as the corresponding isogenic strain in the absence of the phage mutation. In further embodiments, one or more modification(s), e.g., mutation(s) or deletion(s) or other modifications described herein in the genome of a phage does not alter the strain activity (e.g., effector activity or metabolic activity) of the engineered bacterium capable of producing the effector as compared to the corresponding isogenic strain without the phage mutation. In some embodiments, the unmodified or genetically engineered bacteria comprising one or more phage modifications, e.g., mutation(s) or deletion(s) or other modifications described herein, have essentially the same or at least similar bacterial strain activity (e.g., effector activity or metabolic activity) when compared to the corresponding isogenic strain without the phage mutation.

Additionally, in some embodiments, one or more modification(s), e.g., mutation(s) or deletion(s) or other modifications described herein, in the genome of a phage alters, e.g., increases or reduces, the bacterial fitness of the engineered bacterium. In some embodiments, the engineered bacteria comprising one or more phage modifications, e.g., mutation(s) or deletion(s) or other modifications described herein, have altered, e.g., reduced or increased, bacterial fitness as compared to the corresponding isogenic strain without the phage mutation. In some embodiments, the one or more modification(s), e.g., mutation(s) or deletion(s) or other modifications described herein in the genome of a phage alters, e.g., reduces or increases, strain activity (e.g., effector activity or metabolic activity) of the bacterium capable of producing the effector as compared to the corresponding isogenic strain without the phage mutation. In some embodiments, unmodified or genetically engineered bacteria comprising one or more phage modifications, e.g., mutation(s) or deletion(s) or other modifications described herein, have altered, e.g., reduced or increased, bacterial strain activity (e.g., effector activity or metabolic activity) as the corresponding isogenic strain without the phage mutation.

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise a modified phage genome sequence consisting of SEQ ID NO: 281.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

“Hyperphenylalaninemia,” “hyperphenylalaninemic,” and “excess phenylalanine” are used interchangeably herein to refer to increased or abnormally high concentrations of phenylalanine in the body. In some embodiments, a diagnostic signal of hyperphenylalaninemia is a blood phenylalanine level of at least 2 mg/dL, at least 4 mg/dL, at least 6 mg/dL, at least 8 mg/dL, at least 10 mg/dL, at least 12 mg/dL, at least 14 mg/dL, at least 16 mg/dL, at least 18 mg/dL, at least 20 mg/dL, or at least 25 mg/dL. As used herein, diseases associated with hyperphenylalaninemia include, but are not limited to, phenylketonuria, classical or typical phenylketonuria, atypical phenylketonuria, permanent mild hyperphenylalaninemia, nonphenylketonuric hyperphenylalaninemia, phenylalanine hydroxylase deficiency, cofactor deficiency, dihydropteridine reductase deficiency, tetrahydropterin synthase deficiency, and Segawa's disease. Affected individuals can suffer progressive and irreversible neurological deficits, mental retardation, encephalopathy, epilepsy, eczema, reduced growth, microcephaly, tremor, limb spasticity, and/or hypopigmentation (Leonard 2006). Hyperphenylalaninemia can also be secondary to other conditions, e.g., liver diseases.

“Phenylalanine ammonia lyase” and “PAL” are used to refer to a phenylalanine metabolizing enzyme (PME) that converts or processes phenylalanine to trans-cinnamic acid and ammonia. Trans-cinnamic acid has low toxicity and is converted by liver enzymes in mammals to hippuric acid, which is secreted in the urine. PAL may be substituted for the enzyme PAH to metabolize excess phenylalanine. PAL enzyme activity does not require THB cofactor activity. In some embodiments, PAL is encoded by a PAL gene derived from a prokaryotic species. In alternate embodiments, PAL is encoded by a PAL gene derived from a eukaryotic species. In some embodiments, PAL is encoded by a PAL gene derived from a bacterial species, including but not limited to, Achromobacter xylosoxidans, Pseudomonas aeruginosa, Photorhabdus luminescens, Anabaena variabilis, and Agrobacterium tumefaciens. In some embodiments, PAL is encoded by a PAL gene derived from Anabaena variabilis and referred to as “PAL1” herein (Moffitt et al., 2007). In some embodiments, PAL is encoded by a PAL gene derived from Photorhabdus luminescens and referred to as “PAL3” herein (Williams et al., 2005). In some embodiments, PAL is encoded by a PAL gene derived from a yeast species, e.g., Rhodosporidium toruloides (Gilbert et al., 1985). In some embodiments, PAL is encoded by a PAL gene derived from a plant species, e.g., Arabidopsis thaliana (Wanner et al., 1995). Any suitable nucleotide and amino acid sequences of PAL, or functional fragments thereof, may be used.

“Phenylalanine hydroxylase” and “PAH” are used to refer to an enzyme that catalyzes the hydroxylation of the aromatic side chain of phenylalanine to create tyrosine in the human body in conjunction with the cofactor tetrahydrobiopterin. The human gene encoding PAH is located on the long (q) arm of chromosome 12 between positions 22 and 24.2. The amino acid sequence of PAH is highly conserved among mammals. Nucleic acid sequences for human and mammalian PAH are well known and widely available. The full-length human cDNA sequence for PAH was reported in 1985 (Kwok et al. 1985). Active fragments of PAH are also well known (e.g., Kobe et al. 1997).

“L-Aminoacid Deaminase” and “LAAD” are used to refer to an enzyme that catalyzes the stereospecific oxidative deamination of L-amino acids to generate their respective keto acids, ammonia, and hydrogen peroxide. For example, LAAD catalyzes the conversion of phenylalanine to phenylpyruvate. Multiple LAAD enzymes are known in the art, many of which are derived from bacteria, such as Proteus, Providencia, and Morganella, or venom. LAAD is characterized by fast reaction rate of phenylalanine degradation (Hou et al., Appl Microbiol Technol. 2015 October; 99(20):8391-402; “Production of phenylpyruvic acid from L-phenylalanine using an L-amino acid deaminase from Proteus mirabilis: comparison of enzymatic and whole-cell biotransformation approaches”). Most eukaryotic and prokaryotic L-amino acid deaminases are extracellular; however, Proteus species LAAD are localized to the plasma membrane (inner membrane), facing outward into the periplasmic space, in which the enzymatic activity resides. As a consequence of this localization, phenylalanine transport through the inner membrane into the cytoplasm is not required for Proteus LAAD mediated phenylalanine degradation. Phenylalanine is readily taken up through the outer membrane into the periplasm without a transporter, eliminating the need for a transporter to improve substrate availability.

In some embodiments, the genetically engineered bacteria comprise a LAAD gene derived from a bacterial species, including but not limited to, Proteus, Providencia, and Morganella bacteria. In some embodiments, the bacterial species is Proteus mirabilis. In some embodiments, the bacterial species is Proteus vulgaris. In some embodiments, the LAAD encoded by the genetically engineered bacteria is localized to the plasma membrane, facing into the periplasmic space and with the catalytic activity occurring in the periplasmic space.

“Phenylalanine metabolizing enzyme” or “PME” are used to refer to an enzyme which is able to degrade phenylalanine. Any phenylalanine metabolizing enzyme known in the art may be encoded by the genetically engineered bacteria. PMEs include, but are not limited to, phenylalanine hydroxylase (PAH), phenylalanine ammonia lyase (PAL), aminotransferase, L-amino acid deaminase (LAAD), and phenylalanine dehydrogenases.

Reactions with phenylalanine hydroxylases, phenylalanine dehydrogenases or aminotransferases require cofactors, while LAAD and PAL do not require any additional cofactors. In some embodiments, the PME encoded by the genetically engineered bacteria requires a cofactor. In some embodiments, this cofactor is provided concurrently or sequentially with the administration of the genetically engineered bacteria. In other embodiments, the genetically engineered bacteria can produce the cofactor. In some embodiments, the genetically engineered bacteria encode a phenylalanine hydroxylase. In some embodiments, the genetically engineered bacteria encode a phenylalanine dehydrogenase. In some embodiments, the genetically engineered bacteria encode an aminotransferase. In some embodiments, the PME encoded by the genetically engineered bacteria does not require a cofactor. Without wishing to be bound by theory, the lack of need for a cofactor means that the rate of phenylalanine degradation by the enzyme is dependent on the availability of the substrate and is not limited by the availability of the cofactor. In some embodiments, the PME produced by the genetically engineered bacteria is PAL. In some embodiments, the PME produced by the genetically engineered bacteria is LAAD. In some embodiments, the genetically engineered bacteria encode combinations of PMEs.

In some embodiments, the catalytic activity of the PME is dependent on oxygen levels. In some embodiments, the PME is catalytically active under microaerobic conditions. As a non-limiting example, LAAD catalytic activity is dependent on oxygen. In some embodiments, LAAD is active under low oxygen conditions, such as microaerobic conditions. In some embodiments, of the invention, the PME functions at very low levels of oxygen or in the absence of oxygen, e.g. as found in the colon. As a non-limiting example, PAL activity is not dependent on the presence of oxygen.

As used herein, “effector” or “effector molecule” can refers to a molecule, such as a metabolite or a polypeptide, which exerts a desired function. An effector may be encoded by a single gene. For example, a single gene can encode a polypeptide which is secreted or displayed. Alternatively, an effector may be synthesized by a biosynthetic pathway requiring multiple genes, e.g., butyrate. The polypeptides encoded by multiple genes within a biosynthetic pathway, e.g., which synthesizes a metabolite with desirable properties, may also be referred to as effectors. Similarly, polypeptides encoded by multiple genes within a catabolic pathway, e.g., for the breakdown of a toxic metabolite, may also be referred to as effectors. These effector molecules may also be referred to as “therapeutic metabolites”, “therapeutic molecules” or “therapeutic polypeptides”. Other terms that are used interchangeably herein with effector are “polypeptide of interest” or “polypeptides of interest”, “protein of interest”, “proteins of interest”.

As used herein, “payload” refers to one or more polynucleotides and/or polypeptides of interest to be produced by a genetically engineered microorganism, such as a bacterium. In some embodiments, the payload is encoded by a gene or multiple genes or an operon. In some embodiments, the one or more genes and/or operon(s) comprising the payload are endogenous to the microorganism. In some embodiments, the one or more elements of the payload is derived from a different microorganism and/or organism. In some embodiments, the payload is a therapeutic payload. In some embodiments, the payload is encoded by genes for the biosynthesis of a molecule. In some embodiments, the payload is encoded by genes for the metabolism, catabolism, or degradation of a molecule. In some embodiments, the payload is encoded by genes for the importation of a molecule. In some embodiments, the payload is encoded by genes for the exportation of a molecule. In some embodiments, the payload is a regulatory molecule(s), e.g., a transcriptional regulator such as FNR. In some embodiments, the payload comprises a regulatory element, such as a promoter or a repressor. In some embodiments, the payload expression is driven from an inducible promoter, such as from FNRS. In some embodiments, payload expression is driven from a constitutive promoter. In some embodiments, the payload comprises a repressor element, such as a kill switch. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

The present disclosure includes, inter alia, genetically engineered bacteria, pharmaceutical compositions thereof, and methods of modulating and treating disorders associated with hyperphenylalaninemia. In some embodiments, the genetically engineered bacteria comprise a gene encoding non-native phenylalanine ammonia lyase (PAL) and are capable of processing and reducing phenylalanine in a mammal. In some embodiments, the engineered bacteria further comprise a gene encoding a phenylalanine transporter. In some embodiments, the engineered bacteria may also comprise a gene encoding LAAD. The engineered bacteria may also contain one or more gene sequences relating to bio-safety and/or bio-containment, e.g., a kill-switch, gene guard system, and/or auxotrophy. The expression of these gene sequence(s) may be regulated using a variety of promoter systems, such as any of the promoter systems disclosed herein, which promoter may be the same promoter to regulate one or more different genes, may be a different copy of the same promoter to regulate different genes, or may involve the use of different promoters used in combination to regulate the expression of different genes. The use of different regulatory or promoter systems to control gene expression provides flexibility (e.g., the ability to differentially control gene expression under different environmental conditions and/or the ability to differentially control gene expression temporally) and also provides the ability to “fine-tune” gene expression, any or all of which regulation may serve to optimize gene expression and/or growth of the bacteria. The genetically engineered bacteria and pharmaceutical compositions comprising those bacteria may be used to metabolize phenylalanine in the body into non-toxic molecules in order to treat and/or prevent conditions associated with hyperphenylalaninemia, including PKU. In certain aspects, the compositions comprising the genetically engineered bacteria may be used in the methods of the disclosure to treat and/or prevent disorders associated with hyperphenylalaninemia.

Effector molecules also include anti-cancer molecules. “anti-cancer molecule” refers to one or more therapeutic substances or drugs of interest to be produced by a genetically engineered microorganism, e.g., engineered bacteria or engineered oncolytic virus, which are capable of reducing and/or inhibiting cell growth or replication. In some embodiments, the anti-cancer molecule is a therapeutic molecule that is useful for modulating or treating a cancer. In some embodiments, the anti-cancer molecule is a therapeutic molecule encoded by a gene. In alternate embodiments, the anti-cancer molecule is a therapeutic molecule produced by a biochemical or biosynthetic pathway, wherein the biosynthetic or biochemical pathway may optionally be endogenous to the microorganism. In some embodiments, the genetically engineered microorganism is capable of producing two or more anti-cancer molecules. Non-limiting examples of anti-cancer molecules include immune checkpoint inhibitors (e.g., CTLA-4 antibodies, PD-1 antibodies, PDL-1 antibodies), cytotoxic agents (e.g., Cly A, FASL, TRAIL, TNF-alpha), immunostimulatory cytokines and co-stimulatory molecules (e.g., OX40, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAFI, antibodies against immune suppressor molecules, anti-VEGF, Anti-CXR4/CXCL12, anti-GLP1, anti-GLP2, anti-galectin1, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK). In some embodiments, the anti-cancer molecule includes nucleic acid molecules that mediate RNA interference, microRNA response or inhibition, TLR response, antisense gene regulation, target protein binding (aptamer or decoy oligos), gene editing, such as CRISPR interference. In some embodiments, bacteria or virus can be used as vectors to transfer DNA into mammalian cells, e.g., by bactofection (Bernardes et al., 2013).

Non-limiting examples of effector molecules include “anti-inflammation molecules” and/or “gut barrier function enhancer molecules”. Anti-inflammation molecules and/or gut barrier function enhancer molecules include, but are not limited to, short-chain fatty acids, butyrate, propionate, acetate, IL-2, IL-22, superoxide dismutase (SOD), GLP-2 and analogs, GLP-1, IL-10, IL-27, TGF-β1, TGF-β2, N-acylphosphatidylethanolamines (NAPEs), elafin (also called peptidase inhibitor 3 and SKALP), trefoil factor, melatonin, tryptophan, PGD₂, and kynurenic acid, indole metabolites, and other tryptophan metabolites, as well as other molecules disclosed herein. Such molecules may also include compounds that inhibit pro-inflammatory molecules, e.g., a single-chain variable fragment (scFv), antisense RNA, siRNA, or shRNA that neutralizes TNF-α, IFN-γ, IL-1β, IL-6, IL-8, IL-17, and/or chemokines, e.g., CXCL-8 and CCL2. Such molecules also include AHR agonists (e.g., which result in IL-22 production, e.g., indole acetic acid, indole-3-aldehyde, and indole) and PXR agonists (e.g., IPA), as described herein. Such molecules also include HDAC inhibitors (e.g., butyrate), activators of GPR41 and/or GPR43 (e.g., butyrate and/or propionate and/or acetate), activators of GPR109A (e.g., butyrate), inhibitors of NF-kappaB signaling (e.g., butyrate), and modulators of PPARgamma (e.g., butyrate), activators of AMPK signaling (e.g., acetate), and modulators of GLP-1 secretion. Such molecules also include hydroxyl radical scavengers and antioxidants (e.g., IPA). A molecule may be primarily anti-inflammatory, e.g., IL-10, or primarily gut barrier function enhancing, e.g., GLP-2. A molecule may be both anti-inflammatory and gut barrier function enhancing. An anti-inflammation and/or gut barrier function enhancer molecule may be encoded by a single gene, e.g., elafin is encoded by the PI3 gene. Alternatively, an anti-inflammation and/or gut barrier function enhancer molecule may be synthesized by a biosynthetic pathway requiring multiple genes, e.g., butyrate.

Effector molecules also include metabolic effector molecules. “Metabolic effector molecules” and/or “satiety effector molecules” include, but are not limited to, n-acyl-phophatidylethanolamines (NAPEs), n-acyl-ethanolamines (NAEs), ghrelin receptor antagonists, peptide YY3-36, cholecystokinin (CCK) family molecules, CCK58, CCK33, CCK22, CCK8, bombesin family molecules, bombesin, gastrin releasing peptide (GRP), neuromedin B (P), glucagon, GLP-1, GLP-2, apolipoprotein A-IV, amylin, somatostatin, enterostatin, oxyntomodulin, pancreatic peptide, short-chain fatty acids, butyrate, propionate, acetate, serotonin receptor agonists, nicotinamide adenine dinucleotide (NAD), nicotinamide mononucleotide (NMN), nucleotide riboside (NR), nicotinamide, and nicotinic acid (NA). Such molecules may also include compounds that inhibit a molecule that promotes metabolic disease, e.g., a single-chain variable fragment (scFv), antisense RNA, siRNA, or shRNA that inhibits dipeptidyl peptidase-4 (DPP4) or ghrelin receptor. A metabolic and/or satiety effector molecule may be encoded by a single gene, e.g., glucagon-like peptide 1 is encoded by the GLP-1 gene. In some embodiments, the genetically engineered bacteria comprising gene sequences comprising one or more circuits for the production or catabolism of tryptophan and/or one of its metabolites further comprise gene sequences for the expression of one or more metabolic effector molecule and/or satiety effector molecules.

Other non-limiting examples of effector molecules are described in in pending, co-owned International Patent Applications PCT/US2016/34200, filed May 25, 2016, PCT/US2017/013072, filed Jan. 11, 2017, PCT/US2017/016603, filed Feb. 3, 2017, PCT/US2017/016609, filed Feb. 4, 2016, PCT/US2017/017563, filed Feb. 10, 2017, PCT/US2017/017552, filed Feb. 10, 2017, PCT/US2016/044922, filed Jul. 29, 2016, PCT/US2016/049781, filed Aug. 31, 2016, PCT/US2016/37098, filed Jun. 10, 2016, PCT/US2016/069052, filed Dec. 28, 2016, PCT/US2016/32562, filed May 13, 2016, PCT/US2016/062369, filed Nov. 16, 2016, and PCT/US2017/013072, the contents of which are herein incorporated by reference in their entireties.

In certain embodiments, new or improved effectors (e.g., PMEs) can be identified according to methods known in the art or described herein, and are encoded by the genetically engineered bacteria. In some embodiments, the enzyme encoded by the genetically engineered bacteria is a wild type enzyme isolated from a viral, prokaryotic or eukaryotic organism. In some embodiments, the enzyme sequence has been further modified or mutated to increase one or more specific properties of the enzyme, such as stability or catalytic activity.

“Phenylalanine metabolite” refers to a metabolite that is generated as a result of the degradation of phenylalanine. The metabolite may be generated directly from phenylalanine, by the enzyme using phenylalanine as a substrate, or indirectly by a different enzyme downstream in the metabolic pathway, which acts on a phenylalanine metabolite substrate. In some embodiments, phenylalanine metabolites are produced by the genetically engineered bacteria encoding a PME.

In some embodiments, the phenylalanine metabolite results directly or indirectly from PAH activity, e.g., from PAH produced by the genetically engineered bacteria. In some embodiments, the metabolite is tyrosine. In some embodiments, the phenylalanine metabolite accumulates in the blood or the urine of a PKU patient, due to defective PAH activity. Non-limiting examples of such PKU metabolites are phenylpyruvic acid and phenyl-lactic acid. Other examples include phenylacetate, phenylethylamine, and phenylacetyl glutamine.

In some embodiments, the phenylalanine metabolite results directly or indirectly from PAL action, e.g., from PAL produced by the genetically engineered bacteria. Non-limiting examples of such PAL metabolites are trans-cinnamic acid and hippuric acid. In some embodiments, the phenylalanine metabolite results directly or indirectly from LAAD action, e.g., from LAAD produced by the genetically engineered bacteria. Examples of such LAAD metabolites are phenylpyruvate and phenyllactic acid.

“Phenylalanine transporter” is used to refer to a membrane transport protein that is capable of transporting phenylalanine into bacterial cells (see, e.g., Pi et al., 1991). In Escherichia coli, the pheP gene encodes a high affinity phenylalanine-specific permease responsible for phenylalanine transport (Pi et al., 1998). In some embodiments, the phenylalanine transporter is encoded by a pheP gene derived from a bacterial species, including but not limited to, Acinetobacter calcoaceticus, Salmonella enterica, and Escherichia coli. Other phenylalanine transporters include a general amino acid permease, encoded by the aroP gene, transports three aromatic amino acids, including phenylalanine, with high affinity, and is thought, together with PheP, responsible for the lion share of phenylalanine import. Additionally, a low level of phenylalanine transport activity has been traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF. In some embodiments, the phenylalanine transporter is encoded by a aroP gene derived from a bacterial species. In some embodiments, the phenylalanine transporter is encoded by LIV-binding protein and LS-binding protein and LivHMGF genes derived from a bacterial species. In some embodiments, the genetically engineered bacteria comprise more than one type of phenylalanine transporter, selected from pheP, aroP, and the LIV-I/LS system.

“Phenylalanine” and “Phe” are used to refer to an amino acid with the formula C₆H₅CH₂CH(NH₂)COOH. Phenylalanine is a precursor for tyrosine, dopamine, norepinephrine, and epinephrine. L-phenylalanine is an essential amino acid and the form of phenylalanine primarily found in dietary protein; the stereoisomer D-phenylalanine is found is lower amounts in dietary protein; DL-phenylalanine is a combination of both forms. Phenylalanine may refer to one or more of L-phenylalanine, D-phenylalanine, and DL-phenylalanine.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu.

“Operably linked” refers a nucleic acid sequence, e.g., a gene encoding PAL, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region.

A “directly inducible promoter” refers to a regulatory region, wherein the regulatory region is operably linked to a gene encoding an effector molecule (e.g. a phenylalanine metabolizing enzyme, e.g. PAL) in the presence of an inducer of said regulatory region, the effector molecule is expressed. An “indirectly inducible promoter” refers to a regulatory system comprising two or more regulatory regions, for example, a first regulatory region that is operably linked to a gene encoding a first molecule, e.g., a transcriptional regulator, which is capable of regulating a second regulatory region that is operably linked to a gene encoding an effector molecule. In the presence of an inducer of the first regulatory region, the second regulatory region may be activated or repressed, thereby activating or repressing expression of the effector molecule. Both a directly inducible promoter and an indirectly inducible promoter are encompassed by “inducible promoter.”

“Exogenous environmental condition(s)” or “environmental conditions” refer to settings or circumstances under which the promoter described herein is directly or indirectly induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions refer to the presence of molecules or metabolites that are specific to the mammalian gut in a healthy or disease-state, e.g., propionate. In some embodiments, the exogenous environmental conditions are specific to the tumor microenvironment. In some embodiments, exogenous environmental conditions are molecules or metabolites that are specific to the tumor microenvironment. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refers to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the payload (e.g. PME, e.g. PAL or LAAD) or rate of induction of the transporter (e.g. PheP), and overall viability and metabolic activity of the strain during strain production.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic and/or low oxygen conditions by the global transcriptional regulator FNR that is naturally found in Nissle. Under anaerobic and/or low oxygen conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| | Examples of responsive genes, promoters, |
|---|---|
| Transcription factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

As used herein, a “tunable regulatory region” refers to a nucleic acid sequence under direct or indirect control of a transcription factor and which is capable of activating, repressing, derepressing, or otherwise controlling gene expression relative to levels of an inducer. In some embodiments, the tunable regulatory region comprises a promoter sequence. The inducer may be RNS, or other inducer described herein, and the tunable regulatory region may be a RNS-responsive regulatory region or other responsive regulatory region described herein. The tunable regulatory region may be operatively linked to a gene sequence(s) or gene cassette for the production of one or more payloads, e.g., a butyrogenic or other gene cassette or gene sequence(s). For example, in one specific embodiment, the tunable regulatory region is a RNS-derepressible regulatory region, and when RNS is present, a RNS-sensing transcription factor no longer binds to and/or represses the regulatory region, thereby permitting expression of the operatively linked gene or gene cassette. In this instance, the tunable regulatory region derepresses gene or gene cassette expression relative to RNS levels. Each gene or gene cassette may be operatively linked to a tunable regulatory region that is directly or indirectly controlled by a transcription factor that is capable of sensing at least one RNS.

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria of the invention comprise a gene encoding a effector molecule (e.g. PME) that is operably linked to a directly or indirectly inducible promoter that is not associated with said gene in nature, e.g., an FNR promoter operably linked to a gene encoding an effector molecule or a ParaBAD promoter operably linked to a second effector molecule.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_1712074; BBa_1719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

In some embodiments, the genetically engineered bacteria are active (e.g., express one or more payloads (e.g. PME(s)) in the gut. In some embodiments, the genetically engineered bacteria are active (e.g., express one or more payloads) in the large intestine. In some embodiments, the genetically engineered bacteria are active (e.g., express one or more payloads) in the small intestine. In some embodiments, the genetically engineered bacteria are active in the small intestine and in the large intestine. Without wishing to be bound by theory, phenylalanine degradation may be every effective in the small intestine, because amino acid absorption, e.g., phenylalanine absorption, occurs in the small intestine. Through the prevention or reduction of phenylalanine uptake into the blood, increased levels and resulting Phe toxicity can be avoided. Additionally, extensive enterorecirculation of amino acids between the intestine and the body may allow the removal of systemic phenylalanine in PKU (e.g., described by Chang et al., in a rat model of PKU (Chang et al., A new theory of enterorecirculation of amino acids and its use for depleting unwanted amino acids using oral enzyme-artificial cells, as in removing phenylalanine in phenylketonuria; Artif Cells Blood Substit Immobil Biotechnol. 1995; 23(1):1-21)). Phenylalanine from the blood circulates into the small intestine (see, e.g., FIG. 15) and can be cleared by bacteria which are active at this location. In some embodiments, the genetically engineered bacteria transit through the small intestine. In some embodiments, the genetically engineered bacteria have increased residence time in the gut. In some embodiments, the genetically engineered bacteria colonize the small or large intestine. In some embodiments, the genetically engineered bacteria colonize the colon. In some embodiments, the genetically engineered bacteria have increased residence time in the gut. In some embodiments, the genetically engineered bacteria do not colonize the gut.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O₂, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table A summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; lmg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% 02 saturation or less, e.g., 9%, 8%, 7%, 6%, 5%, 4%, 3%, 2%, 1%, 0%, 02 saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of 02 saturation levels between 0-9%, inclusive (e.g., 0-5%, 0.05-0.1%, 0.1-0.2%, 0.1-0.5%, 0.5-2.0%, 0-8%, 5-7%, 0.3-4.2% 02, etc.). The exemplary fractions and ranges listed here are for illustrative purposes and not meant to be limiting in any way.

TABLE A
Intestinal Oxygen Tension

| Compartment | Oxygen Tension |
|---|---|
| stomach | ~60 torr (e.g., 58 +/− 15 torr) |
| duodenum and first | ~30 torr (e.g., 32 +/− 8 torr); |
| part of jejunum | ~20% oxygen in ambient air |
| Ileum (mid- small | ~10 torr; ~6% oxygen in ambient air |
| intestine) | (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

In some embodiments, a promoter described herein is directly or indirectly induced by conditions in a culture vessel (e.g., a flask or a fermenter or other appropriate culture vessel), in which the strain is grown or maintained prior to in vivo administration. Non-limiting examples of such conditions which are provided during culture of the strain prior to in vivo administration include low oxygen, anaerobic, microaerobic, or aerobic conditions, other defined oxygen levels (such as those exemplified below), presence of arabinose, presence of IPTG, rhamnose or other chemical and/or nutritional inducers described herein or known in the art. In some embodiments, the conditions in a culture vessel are set at certain oxygen levels, e.g., between 1% and 10% oxygen, between 10% and 20% oxygen, between 20% and 30% oxygen, between 30% and 40% oxygen, between 40% and 50% oxygen, between 60% and 70% oxygen, between 70% and 80% oxygen, between 80% and 90% oxygen, between 90% and 100% oxygen, and other levels of oxygen as described herein, at which point the promoter is directly or indirectly induced.

As used herein, the term “gene” or “gene sequence” is meant to refer to a genetic sequence, e.g., a nucleic acid sequence. The gene, gene sequence or genetic sequence is meant to include a complete gene sequence or a partial gene sequence. The gene, gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., a gene encoding an effector molecule, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising a gene encoding an effector molecule (e.g., a PAL), in which the plasmid or chromosome carrying the effector gene is stably maintained in the host cell, such that the effector can be expressed in the host cell, and the host cell is capable of survival and/or growth in vitro and/or in vivo. In some embodiments, copy number affects the stability of expression of the non-native genetic material, e.g. a PAL gene. In some embodiments, copy number affects the level of expression of the non-native genetic material, e.g. a PAL gene or a PAH gene.

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. Treating a disease, disorder, or condition may encompass reducing or eliminating an associated symptom without necessarily encompassing the elimination of the underlying disease. For example, primary hyperphenylalaninemia is caused by inborn genetic mutations for which there are no known cures. Hyperphenylalaninemia can also be secondary to other conditions, e.g., liver diseases. Treating hyperphenylalaninemia may encompass reducing or eliminating excess phenylalanine and/or associated symptoms, and does not necessarily encompass the elimination of the underlying disease. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease.

As used herein a “pharmaceutical composition” refers to a preparation of genetically engineered bacteria of the invention with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disease or condition. A therapeutically effective amount, as well as a therapeutically effective frequency of administration, can be determined by methods known in the art and discussed below.

As used herein, the term “antibody” or “antibodies” is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, VHH fragments), heavy chain antibodies (HCAb), nanobodies, diabodies, and minibodies. Antibodies can have more than one binding specificity, e.g. be bispecific. The term “antibody” is also meant to include so-called antibody mimetics. Antibody mimetics refers to small molecules, e.g., 3-30 kDa, which can be single amino acid chain molecules, which can specifically bind antigens but do not have an antibody-related structure. Antibody mimetics, include, but are not limited to, Affibody molecules (Z domain of Protein A), Affilins (Gamma-B crystalline), Ubiquitin, Affimers (Cystatin), Affitins (Sac7d (from Sulfolobus acidocaldarius), Alphabodies (Triple helix coiled coil), Anticalins (Lipocalins), Avimers (domains of various membrane receptors), DARPins (Ankyrin repeat motif), Fynomers (SH3 domain of Fyn), Kunitz domain peptides Kunitz domains of various protease inhibitors), Ecallantide (Kalbitor), and Monobodies. In certain aspects, the term “antibody” or “antibodies” is meant to refer to a single chain antibody(ies), single domain antibody(ies), and camelid antibody(ies). Utility of antibodies in the treatment of cancer and additional anti cancer antibodies can for example be found in Scott et al., Antibody Therapy for Cancer, Nature Reviews Cancer April 2012 Volume 12, incorporated by reference in its entirety.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778. The Vh and VL sequences of the scFv can be connected via the N-terminus of the VH connecting to the C-terminus of the VL or via the C-terminus of the VH connecting to the N-terminus of the VL. ScFv fragments are independent folding entities that can be fused indistinctively on either end to other epitope tags or protein domains. Linkers of varying length can be used to link the Vh and VL sequences, which the linkers can be glycine rich (provides flexibility) and serine or threonine rich (increases solubility). Short linkers may prevent association of the two domains and can result in multimers (diabodies, tribodies, etc.). Long linkers may result in proteolysis or weak domain association (described in Voelkel et al el., 2011). Linkers of length between 15 and 20 amino acids or 18 and 20 amino acids are most often used. Additional non-limiting examples of linkers, including other flexible linkers are described in Chen et al., 2013 (Adv Drug Deliv Rev. 2013 Oct. 15; 65(10): 1357-1369. Fusion Protein Linkers: Property, Design and Functionality), the contents of which is herein incorporated by reference in its entirety. Flexible linkers are also rich in small or polar amino acids such as Glycine and Serine, but can contain additional amino acids such as Threonine and Alanine to maintain flexibility, as well as polar amino acids such as Lysine and Glutamate to improve solubility. Exemplary linkers include, but are not limited to, (Gly-Gly-Gly-Gly-Ser)n, KESGSVSSEQLAQFRSLD and EGKSSGSGSESKST, (Gly)8, and Gly and Ser rich flexible linker, GSAGSAAGSGEF. “Single chain antibodies” as used herein also include single-domain antibodies, which include camelid antibodies and other heavy chain antibodies, light chain antibodies, including nanobodies and single domains VH or VL domains derived from human, mouse or other species. Single domain antibodies may be derived from any species including, but not limited to mouse, human, camel, llama, fish, shark, goat, rabbit, and bovine. Single domain antibodies include domain antigen-binding units which have a camelid scaffold, derived from camels, llamas, or alpacas. Camelids produce functional antibodies devoid of light chains. The heavy chain variable (VH) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies. Camelid scaffold-based antibodies can be produced using methods well known in the art. Cartilaginous fishes also have heavy-chain antibodies (IgNAR, ‘immunoglobulin new antigen receptor’), from which single-domain antibodies called VNAR fragments can be obtained. Alternatively, the dimeric variable domains from IgG from humans or mice can be split into monomers. Nanobodies are single chain antibodies derived from light chains. The term “single chain antibody” also refers to antibody mimetics.

In some embodiments, the antibodies expressed by the engineered microorganisms are bispecific. In certain embodiments, a bispecific antibody molecule comprises a scFv, or fragment thereof, have binding specificity for a first epitope and a scFv, or fragment thereof, have binding specificity for a second epitope. Antigen-binding fragments or antibody portions include bivalent scFv (diabody), bispecific scFv antibodies where the antibody molecule recognizes two different epitopes, single binding domains (dAbs), and minibodies. Monomeric single-chain diabodies (scDb) are readily assembled in bacterial and mammalian cells and show improved stability under physiological conditions (Voelkel et al., 2001 and references therein; Protein Eng. (2001) 14 (10): 815-823 (describes optimized linker sequences for the expression of monomeric and dimeric bispecific single-chain diabodies).

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cysm Ser, Tyr, Thr; -Val, Ile, Len, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. The term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. In some embodiments, the improvement of transcription and/or translation involves increasing the level of transcription and/or translation. In some embodiments, the improvement of transcription and/or translation involves decreasing the level of transcription and/or translation. In some embodiments, codon optimization is used to fine-tune the levels of expression from a construct of interest. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent, inter alia, on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the protein(s) of interest or therapeutic protein(s) from the microbial, e.g., bacterial cytoplasm. The secretion system may comprise a single protein or may comprise two or more proteins assembled in a complex e.g., HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the proteins of interest include a “secretion tag” of either RNA or peptide origin to direct the protein(s) of interest or therapeutic protein(s) to specific secretion systems. In some embodiments, the secretion system is able to remove this tag before secreting the protein(s) of interest from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the protein(s) of interest into the extracellular milieu.]]

As used herein, the term “transporter” is meant to refer to a mechanism, e.g., protein or proteins, for importing a molecule, e.g., amino acid, toxin, metabolite, substrate, etc. into the microorganism from the extracellular milieu. For example, a phenylalanine transporter such as PheP imports phenylalanine into the microorganism.

Effectors also include immune checkpoint inhibitors. An “immune checkpoint inhibitor” or “immune checkpoint” refers to a molecule that completely or partially reduces, inhibits, interferes with, or modulates one or more immune checkpoint proteins. Immune checkpoint proteins regulate T-cell activation or function, and are known in the art. Non-limiting examples include CTLA-4 and its ligands CD 80 and CD86, and PD-1 and its ligands PD-L1 and PD-L2. Immune checkpoint proteins are responsible for co-stimulatory or inhibitory interactions of T-cell responses, and regulate and maintain self-tolerance and physiological immune responses. Systemic immunotherapy, e.g., using CTLA-4 inhibitors, may alter immunoregulation, provoke immune dysfunction, and result in opportunistic autoimmune disorders (see, e.g., Kong et al., 2014).

As used herein, a genetically engineered microorganism, e.g., engineered bacterium or phage, or molecule that “inhibits” a biological molecule refers to a bacterium or virus or molecule that is capable of reducing, decreasing, or eliminating the biological activity, biological function, and/or number of that biological molecule, as compared to control, e.g., an untreated control or an unmodified microorganism of the same subtype under the same conditions.

As used herein, a genetically engineered microorganism, e.g., engineered bacterium or phage molecule that “activates” or “stimulates” a biological molecule, refers to a bacterium or phage molecule that is capable of activating, increasing, enhancing, or promoting the biological activity, biological function, and/or number of that biological molecule, as compared to control, e.g., an untreated control or an unmodified microorganism of the same subtype under the same conditions.

The terms “phage” and “bacteriophage” are used interchangeably herein. Both terms refer to a virus that infects and replicates within a bacterium. As used herein “phage” or bacteriophage” collectively refers to prophage, lysogenic, dormant, temperate, intact, defective, cryptic, and satellite phage, phage tail bacteriocins, tailiocins, and gene transfer agents.

As used therein the term “prophage” refers to the genomic material of a bacteriophage, which is integrated into a replicon of the host cell and replicates along with the host. The prophage may be able to produce phages if specifically activated. In some cases, the prophage is not able to produce phages or has never done so (i.e., defective or cryptic prophages). In some cases, prophage also refers to satellite phages. The terms “prophage” and “endogenous phage” are used interchangeably herein.

As used herein, the term “temperate phage” or “temperate bacteriophage” or “prophage” are used interchangeably to refer to a phage which exists within the DNA of the bacterial host and replicate along with the host during the bacterial replication cycle and cell division.

As used herein the term “natural state” of a bacterium or organism or “native state” of a bacterium or refers to an organism which has not been modified by genetic engineering. In some cases, the term “natural state” of a bacterium or organism or “native state” of a bacterium refers to an organism which has not been modified by genetic engineering as compared to an isogenic strain that has been modified with respect to a defined element. As such, the bacterium may be in its natural state with respect to one defined element, but not in its natural state with respect to another defined element. In some embodiments, a bacterium may comprise one or more of the same or different phage(s) or prophage(s) in its natural or native state. In some embodiments, a bacterium, which in its native or natural state comprises one or more of the same or different types of phages or prophages, serves a progenitor strain for an engineered strain. Consequently, the same one or more endogenous phage(s) or prophage(s) may also be present in a genetically engineered bacterium, e.g., if the progenitor or parental strain contained such an endogenous phage or prophage in its native state. As such the genetically engineered bacterium also contains the prophage in its natural state (wherein the phage is the defined element that is in its natural state).

“Endogenous phage” or “endogenous prophage” also refers to a phage that is present in the natural state of a bacterium (and its parental strain).

As used herein the term “phage knockout” or “inactivated phage” refers to a phage which has been modified so that it can either no longer produce and/or package phage particles or it produces fewer phage particles than the wild type phage sequence. In some embodiments, the inactivated phage or phage knockout refers to the inactivation of a temperate phage in its lysogenic state, i.e., to a prophage. Such a modification refers to a mutation in the phage; such mutations include insertions, deletions (partial or complete deletion of phage genome), substitutions, inversions, at one or more positions within the phage genome, e.g., within one or more genes within the phage genome.

As used herein the term “isogenic” bacterial strains refers to bacterial strains that are genetically identical or that contain defined changes but are otherwise identical. For example, isogenic mutants typically refers to two strains that are identical except that one contains a defined mutation in one or more known genes or proteins. As such, a phage free or phage less strain has a corresponding isogenic strain which contains prophage which can be induced and release phage particles from the bacterial cell.

As used herein the adjectives “phage-free”, “phage free” and “phageless” are used interchangeably to characterize a bacterium or strain which contains one or more prophages, one or more of which have been modified. The modification can result in a loss of the ability of the prophage to be induced or release phage particles. Alternatively, the modification can result in less efficient or less frequent induction or less efficient or less frequent phage release as compared to the isogenic strain without the modification. Ability to induce and release phage can be measured using a plaque assay as described herein.

As used herein, the term “lysogen” refers to a bacterium containing a prophage, which is in the lysogenic cycle, in which the phage genes required for lysis are not expressed.

As used herein phage induction refers to the part of the life cycle of a lysogenic prophage, in which the lytic phage genes are activated, phage particles are produced and lysis occurs.

As used herein, the term induction refers to the conversion of a lysogenic infection into a productive infection, i.e., the induced prophage initiates the production and release of phage particles. Induction often is stimulated by damage to bacterial DNA, and may or may not involve excision of the prophage from the bacterial chromosome.

In some embodiments, the genetically engineered bacteria are useful for the treatment, prevention, management, reduction in severity of, amelioration, cure a disorder, disease or condition. In some embodiments, the disorder is an autoimmune disorder. As used herein, “autoimmune disorders” include, but are not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis. In some embodiments, the disorder is graft vs host disease.

In some embodiments, the disease is a metabolic disease. As used herein, “metabolic diseases” include, but are not limited to, type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

In some embodiments, the disorder is cancer. “Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute). In some embodiments, the disorder is a hyperammonemia disorder.

In some embodiments, the disorders are rare diseases, including but not limited to, hyperammonemia, ureacycle disorders, propionic acidemia, methylmalonic acidemia, maple syrup urine disease, isovaleric acidemia, hyperoxaluria, phenylketonurea.

Exemplary circuitry for the treatment, prevention, reduction in severity, management, amelioration, cure of one or more of the disorders described above are described in pending, co-owned International Patent Applications PCT/US2016/34200, filed May 25, 2016, PCT/US2017/013072, filed Jan. 11, 2017, PCT/US2017/016603, filed Feb. 3, 2017, PCT/US2017/016609, filed Feb. 4, 2016, PCT/US2017/017563, filed Feb. 10, 2017, PCT/US2017/017552, filed Feb. 10, 2017, PCT/US2016/044922, filed Jul. 29, 2016, PCT/US2016/049781, filed Aug. 31, 2016, PCT/US2016/37098, filed Jun. 10, 2016, PCT/US2016/069052, filed Dec. 28, 2016, PCT/US2016/32562, filed May 13, 2016, PCT/US2016/062369, filed Nov. 16, 2016, and PCT/US2017/013072, the contents of which are herein incorporated by reference in their entireties.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

In some embodiments, the bacteria disclosed herein contain one or more mutations or modifications to an endogenous phage genome. In some embodiments, the bacterium comprises the bacteriophage in its natural or native state. In some embodiments, the phage is present in all isolates of a particular bacterium. In some embodiments, the phage is present in bacteria of the same species, strain, or substrain. In some embodiments, the phage is an intact prophage. In some embodiments, the phage is a defective prophage. In some embodiments, the one or more mutations renders the phage unable to enter the lytic cycle. In some embodiments, the one or more mutations affect the ability of the phage to undergo the lytic cycle, e.g., reduce the frequency or reduce the number of bacteria in a given population that can undergo the lytic stage. In some embodiments, the one or more mutations prevent the phage from infecting other bacteria. In some embodiments, the one or more mutations alters, e.g., increases or reduces, bacterial fitness. In some embodiments, the one or more mutations alters e.g., increases or reduces, effector function. In some embodiments, the one or more mutations do not alter bacterial fitness. In some embodiments, the one or more mutations do not alter effector function. In some embodiments, the one or more mutations improve the process by which the bacteria is manufactured or produced, including large-scale manufacturing. In any of these embodiments, the bacterium may otherwise be in its natural state. Alternatively, in any of these embodiments, the bacteria may be further genetically engineered to include gene sequence encoding one or more effector molecules.

In some embodiments, a bacterium comprising one or more mutated phages can be used as a bacterial chassis, to which genetic circuitry is added or modified.

In some embodiments, the bacteria are non-pathogenic bacteria. In some embodiments, the bacteria are commensal bacteria. In some embodiments, the bacteria are probiotic bacteria. In some embodiments, the bacteria are naturally pathogenic bacteria that are modified or mutated to reduce or eliminate pathogenicity. In some embodiments, non-pathogenic bacteria are Gram-negative bacteria. In some embodiments, non-pathogenic bacteria are Gram-positive bacteria. Exemplary bacteria include, but are not limited to, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In some embodiments, the bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and is not uropathogenic (Sonnenborn et al., 2009). As early as in 1917, E. coli Nissle was packaged into medicinal capsules, called Mutaflor, for therapeutic use. It is commonly accepted that E. coli Nissle's therapeutic efficacy and safety have convincingly been proven (Ukena et al., 2007).

In some embodiments, the bacteria of the disclosure or tumor-targeting bacteria. Tumor-targeting bacteria are described are described in International Patent Application PCT/US2017/013072, filed Jan. 11, 2017, published as WO2017/123675, the contents of which is herein incorporated by reference in its entirety.

One of ordinary skill in the art would appreciate that the genetic modifications disclosed herein may be adapted for other species, strains, and subtypes of bacteria. Furthermore, genes from one or more different species can be introduced into one another, e.g., the PAL gene from Rhodosporidium toruloides can be expressed in Escherichia coli (Sarkissian et al., 1999).

In any of these embodiments, any of the bacterial species disclosed herein or known in the art, and which may be used according to the disclosure, contain one or more mutations or modifications to one or more endogenous phage genomes. In some embodiments, the modifications to the endogenous phage genomes comprise one or more deletion(s), insertion(s), substitution(s) or inversions(s) or combinations thereof within the phage genomes. In some embodiments, the modification(s) is one or more deletions in the phage genome(s). In some embodiments, one or more phage genes are deleted. In some embodiments, one or more phage genes are partially deleted. In some embodiments, the modification(s) is one or more insertions in the phage genome(s). In some embodiments, the insertion comprises gene sequence encoding an antibiotic cassette as described herein. In some embodiments, one or more genes in the phage genome(s) are substituted with alternate gene sequence(s). In some embodiments, the substitution comprises gene sequence encoding an antibiotic cassette. In some embodiments, the entire sequence(s) of one or more phage genes is inverted. In some embodiments a partial sequence of one or more phage genes are inverted.

Unmodified E. coli Nissle and the genetically engineered bacteria of the invention may be destroyed, e.g., by defense factors in the gut or blood serum (Sonnenborn et al., 2009) or by activation of a kill switch, several hours or days after administration. Thus, the genetically engineered bacteria may require continued administration. In some embodiments, the residence time is calculated for a human subject. Residence time in vivo may be calculated for the genetically engineered bacteria of the invention (see, e.g., FIG. 68 of WO2017087580, the contents of which are herein incorporated by reference in their entirety).

In some embodiments, the genetically engineered bacteria comprise a gene encoding PAL, wherein the PAL gene is operably linked to a directly or indirectly inducible promoter. In some embodiments, the bacteria comprise a non-native PAL gene. In some embodiments, the bacteria comprise additional copies of a native PAL gene. In some embodiments, the promoter is not associated with the PAL gene in nature. In some embodiments, the promoter is any one or more of the promoters disclosed herein.

In some embodiments, the genetically engineered bacteria comprise a gene encoding PAH, wherein the PAH gene is operably linked to a directly or indirectly inducible promoter. In some embodiments, the bacteria comprise a non-native PAH gene. In some embodiments, the bacteria comprise additional copies of a native PAH gene. In some embodiments, the promoter is not associated with the PAH gene in nature. In some embodiments, the promoter is any one or more of the promoters disclosed herein.

In some embodiments, the genetically engineered bacteria comprise a gene encoding LAAD, wherein the LAAD gene is operably linked to a directly or indirectly inducible promoter. In some embodiments, the bacteria comprise a non-native LAAD gene. In some embodiments, the bacteria comprise additional copies of a native LAAD gene. In some embodiments, the promoter is not associated with the LAAD gene in nature. In some embodiments, the promoter is any one or more of the promoters disclosed herein.

In some embodiments, the genetically engineered bacteria further comprise a gene encoding a phenylalanine transporter (PheP). In certain embodiments, the bacteria comprise additional copies of a native gene encoding a phenylalanine transporter, wherein the phenylalanine transporter gene is operably linked to a directly or indirectly inducible promoter. In alternate embodiments, the bacteria comprise a gene encoding a non-native phenylalanine transporter, wherein the phenylalanine transporter gene is operably linked to a directly or indirectly inducible promoter. Both embodiments are encompassed by the term “non-native” phenylalanine transporter. In some embodiments, the promoter is not associated with the pheP gene in nature. In some embodiments, the same promoter controls expression of PheP and PAL and/or PAH and/or LAAD. In some embodiments, the promoter that controls expression of PheP differs from the promoter that controls expression of PAL and/or PAH and/or LAAD. In some embodiments, the promoter that controls the expression of PheP is any one or more of the promoters disclosed herein.

In some embodiments, the promoter that is operably linked to PAL, PAH, LAAD, and/or pheP is directly or indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by exogenous environmental conditions specific to the large intestine of a mammal. In some embodiments, the promoter is directly or indirectly induced by low-oxygen or anaerobic and/or low oxygen conditions such as the environment of the mammalian gut. In some embodiments, the promoter is directly or indirectly induced by the presence of molecules or metabolites that are specific to the gut of a mammal, e.g., propionate. In some embodiments, the promoter is directly or indirectly induced by exposure to tetracycline. In some embodiments, the promoter is directly or indirectly induced by exposure to arabinose. In some embodiments, the promoter is directly or indirectly induced by exposure to IPTG. In some embodiments, the promoter is directly or indirectly induced by exposure to rhamnose or other chemical and/or nutritional inducer known in the art. In some embodiments, the promoter is directly or indirectly regulated by the exogenous environmental temperature. In some embodiments, the promoter is directly or indirectly induced by exposure to IPTG or other lacI binding compound. In some embodiments, the promoter is directly or indirectly induced by exposure to rhamnose. In some embodiments, the promoter is directly or indirectly induced by increase in temperature. In some embodiments, the promoter is directly or indirectly induced by decrease in temperature. In some embodiments, the promoter is directly or indirectly induced by a molecule that is co-administered with the genetically engineered bacteria of the invention. Such a molecule may be tetracycline or IPTG or arabinose or other chemical and/or nutritional inducer known in the art.

In some embodiments, the promoter is directly or indirectly induced prior to in vivo administration. Non-limiting examples of such conditions which are provided during culture of the strain prior to in vivo administration include low oxygen, anaerobic, microaerobic, or aerobic conditions, other defined oxygen levels (such as those exemplified below), presence of arabinose, presence of IPTG, rhamnose or other chemical and/or nutritional inducers described herein or known in the art. In some embodiments, the conditions in a culture vessel are set at certain oxygen levels, e.g between 1% and 10% oxygen, between 10% and 20% oxygen, between 20% and 30% oxygen, between 30% and 40% oxygen, between 40% and 50% oxygen, between 60% and 70% oxygen, between 70% and 80% oxygen, between 80% and 90% oxygen, between 90% and 100% oxygen, and other levels of oxygen as described herein, at which point the promoter is directly or indirectly induced.

### Bacteriophages

In some embodiments, the bacteria of the disclosure comprise one or more lysogenic, dormant, temperate, intact, defective, cryptic, or satellite phage or bacteriocins/phage tail or gene transfer agents in their natural state. In some embodiments, the prophage or bacteriophage exists in all isolates of a particular bacterium of interest. In some embodiments, the bacteria are probiotic bacteria. In some embodiments, the bacteria are genetically engineered derivatives of a parental strain comprising one or more of such bacteriophage. Accordingly, such bacteria of the disclosure may be in their natural state or be further genetically modified to contain circuitry for the expression or production of one or more effector molecules. In any of the embodiments described herein, the bacteria comprise one or more modifications or mutations within a prophage or bacteriophage genome which alters the properties or behavior of the bacteriophage. In some embodiments, the modifications or mutations prevent the prophage from entering or completing the lytic process. In some embodiments, the modifications or mutations prevent the phage from infecting other bacteria of the same or a different type.

In some embodiments, the modifications or mutations alter, e.g., reduce or increase, the fitness of the bacterial host. In some embodiments, the modifications or mutations alter, e.g., reduce or increase, desired effector function, e.g., of a genetically engineered bacterium. In some embodiments, the modifications or mutations do not alter, e.g., reduce or increase, the fitness of the bacterial host. In some embodiments, the modifications or mutations do not alter, e.g., reduce or increase, desired effector function, e.g., of a genetically engineered bacterium.

Phage genome size varies enormously, ranging from the smallest Leuconostoc phage L5 (2,435 bp), ˜11.5 kbp (e.g. Mycoplasma phage P1), ˜21 kbp (e.g. Lactococcus phage c2), and ˜30 kbp (e.g. Pasteurella phage F108) to the almost 500 kbp genome of Bacillus megaterium phage G (Hatfull and Hendrix; Bacteriophages and their Genomes, Curr Opin Virol. 2011 Oct. 1; 1(4): 298-303, and references therein). Phage genomes may encode less than 10 genes up to several hundreds of genes. Temperate phages or prophages are typically integrated into the chromosome(s) of the bacterial host, although some examples of phages that are integrated into bacterial plasmids also exist (Little, Loysogeny, Prophage Induction, and Lysogenic Conversion. In: Waldor M K, Friedman D I, Adhya S, editors. Phages Their Role in Bacterial Pathogenesis and Biotechnology. Washington DC: ASM Press; 2005. pp. 37-54). In some cases, the phages are always located at the same position within the bacterial host chromosome(s), and this position is specific to each phage, i.e., different phages are located at different positions. Other phages are more permissive in that they can integrate at numerous different locations.

Accordingly, the bacteria of the disclosure comprise one or more phages genomes which may vary in length. In one embodiment, the genetically engineered bacteria comprise a bacteriophage genome ranging in length from at least about 1 bp to 10 kb. In one embodiment, the bacteria comprise a bacteriophage genome ranging in length from at least about 1 bp to 10 kb. In one embodiment, the genetically engineered bacteria comprise a bacteriophage genome ranging in length from at least about 10 kb to 20 kb. In one embodiment, the genetically engineered bacteria comprise a bacteriophage genome ranging in length from at least about 20 kb to 30 kb. In one embodiment, the genetically engineered bacteria comprise a bacteriophage genome ranging in length from at least about 30 kb to 40 kb. In one embodiment, the genetically engineered bacteria comprise a bacteriophage genome ranging in length from at least about 30 kb to 40 kb. In one embodiment, the genetically engineered bacteria comprise a bacteriophage genome ranging in length from at least about 40 kb to 50 kb. In one embodiment, the genetically engineered bacteria comprise a bacteriophage genome ranging in length from at least about 50 kb to 60 kb. In one embodiment, the genetically engineered bacteria comprise a bacteriophage genome ranging in length from at least about 60 kb to 70 kb. In one embodiment, the genetically engineered bacteria comprise a bacteriophage genome ranging in length from at least about 70 kb to 80 kb. In one embodiment, the genetically engineered bacteria comprise a bacteriophage genome ranging in length from at least about 80 kb to 90 kb. In one embodiment, the genetically engineered bacteria comprise a bacteriophage genome ranging in length from at least about 90 kb to 100 kb. In one embodiment, the genetically engineered bacteria comprise a bacteriophage genome ranging in length from at least about 100 kb to 120 kb. In one embodiment, the genetically engineered bacteria comprise a bacteriophage genome ranging in length from at least about 120 kb to 140 kb. In one embodiment, the genetically engineered bacteria comprise a bacteriophage genome ranging in length from at least about 140 kb to 160 kb. In one embodiment, the genetically engineered bacteria comprise a bacteriophage genome ranging in length from at least about 160 kb to 180 kb. In one embodiment, the genetically engineered bacteria comprise a bacteriophage genome ranging in length from at least about 180 kb to 200 kb. In one embodiment, the genetically engineered bacteria comprise a bacteriophage genome ranging in length from at least about 200 kb to 180 kb. In one embodiment, the genetically engineered bacteria comprise a bacteriophage genome ranging in length from at least about 160 kb to 250 kb. In one embodiment, the genetically engineered bacteria comprise a bacteriophage genome ranging in length from at least about 250 kb to 300 kb. In one embodiment, the genetically engineered bacteria comprise a bacteriophage genome ranging in length from at least about 300 kb to 350 kb. In one embodiment, the genetically engineered bacteria comprise a bacteriophage genome ranging in length from at least about 350 kb to 400 kb. In one embodiment, the genetically engineered bacteria comprise a bacteriophage genome ranging in length from at least about 400 kb to 500 kb. In one embodiment, the genetically engineered bacteria comprise a bacteriophage genome ranging in length from at least about 500 kb to 1000 kb. In one embodiment, the genetically engineered bacteria comprise a bacteriophage genome greater than 1000 kb in length.

In some embodiments, the bacteria of the disclosure comprise one or more phages genomes, which comprise one or more genes encoding one or more polypeptides. In one embodiment, the genetically engineered bacteria comprise a bacteriophage genome comprising at least about 1 to 5 genes. In one embodiment, the genetically engineered bacteria comprise a bacteriophage genome comprising at least about 5 to 10 genes. In one embodiment, the genetically engineered bacteria comprise a bacteriophage genome comprising at least about 10 to 15 genes. In one embodiment, the genetically engineered bacteria comprise a bacteriophage genome comprising at least about 15 to 20 genes. In one embodiment, the genetically engineered bacteria comprise a bacteriophage genome comprising at least about 20 to 25 genes. In one embodiment, the genetically engineered bacteria comprise a bacteriophage genome comprising at least about 25 to 30 genes. In one embodiment, the genetically engineered bacteria comprise a bacteriophage genome comprising at least about 30 to 35 genes. In one embodiment, the genetically engineered bacteria comprise a bacteriophage genome comprising at least about 35 to 40 genes. In one embodiment, the genetically engineered bacteria comprise a bacteriophage genome comprising at least about 40 to 45 genes. In one embodiment, the genetically engineered bacteria comprise a bacteriophage genome comprising at least about 45 to 50 genes. In one embodiment, the genetically engineered bacteria comprise a bacteriophage genome comprising at least about 50 to 55 genes. In one embodiment, the genetically engineered bacteria comprise a bacteriophage genome comprising at least about 55 to 60 genes. In one embodiment, the genetically engineered bacteria comprise a bacteriophage genome comprising at least about 60 to 65 genes. In one embodiment, the genetically engineered bacteria comprise a bacteriophage genome comprising at least about 65 to 70 genes. In one embodiment, the genetically engineered bacteria comprise a bacteriophage genome comprising at least about 70 to 75 genes. In one embodiment, the genetically engineered bacteria comprise a bacteriophage genome comprising at least about 75 to 80 genes. In one embodiment, the genetically engineered bacteria comprise a bacteriophage genome comprising at least about 80 to 85 genes. In one embodiment, the genetically engineered bacteria comprise a bacteriophage genome comprising at least about 85 to 90 genes. In one embodiment, the genetically engineered bacteria comprise a bacteriophage genome comprising at least about 90 to 95 genes. In one embodiment, the genetically engineered bacteria comprise a bacteriophage genome comprising at least about 95 to 100 genes. In one embodiment, the genetically engineered bacteria comprise a bacteriophage genome comprising at least about 100 to 115 genes. In one embodiment, the genetically engineered bacteria comprise a bacteriophage genome comprising at least about 115 to 120 genes. In one embodiment, the genetically engineered bacteria comprise a bacteriophage genome comprising at least about 120 to 125 genes. In one embodiment, the genetically engineered bacteria comprise a bacteriophage genome comprising at least about 125 to 130 genes. In one embodiment, the genetically engineered bacteria comprise a bacteriophage genome comprising at least about 130 to 135 genes. In one embodiment, the genetically engineered bacteria comprise a bacteriophage genome comprising at least about 135 to 140 genes. In one embodiment, the genetically engineered bacteria comprise a bacteriophage genome comprising at least about 140 to 145 genes. In one embodiment, the genetically engineered bacteria comprise a bacteriophage genome comprising at least about 145 to 150 genes. In one embodiment, the genetically engineered bacteria comprise a bacteriophage genome comprising at least about 150 to 160 genes. In one embodiment, the genetically engineered bacteria comprise a bacteriophage genome comprising at least about 160 to 170 genes. In one embodiment, the genetically engineered bacteria comprise a bacteriophage genome comprising at least about 170 to 180 genes. In one embodiment, the genetically engineered bacteria comprise a bacteriophage genome comprising at least about 180 to 190 genes. In one embodiment, the genetically engineered bacteria comprise a bacteriophage genome comprising at least about 190 to 200 genes. In one embodiment, the genetically engineered bacteria comprise a bacteriophage genome comprising at least about 200 to 300 genes. In one embodiment, the genetically engineered bacteria comprise a bacteriophage genome comprising more than about 300 genes.

In some embodiments, the phage is always or almost always located at the same location or position within the bacterial host chromosome(s) in a particular species. In some embodiments, the phages are found integrated at different locations within the host chromosome in a particular species. In some embodiments, the phage is located on a plasmid.

The presence of prophage sequences may also confer certain properties to the bacteria which are not present in an isogenic strain without the phage. For example, the prophage may in some cases allow bacteria to acquire antibiotic resistance, to exist in new environmental niches, to improve adhesion or to become pathogenic. Additionally, through the lytic process, DNA from one bacterium can be picked up and released in another bacterium, and phages therefore function as a vehicle for gene transfer.

Accordingly, in some embodiments, the bacteria comprise a phage which bestows antibiotic resistance to the bacterium. In some embodiments, the bacteria comprise a phage which bestows additional fitness to the bacterium. In some embodiments, the bacteria comprise a phage which bestows ability to grow in new environments to the bacterium. In some embodiments, the bacteria comprise a phage which bestows the ability to transfer host genetic material to another bacterium of the same or different species.

In some embodiments, the prophage may be a defective or a cryptic prophage. Defective prophages can no longer undergo a lytic cycle. Cryptic prophages may not be able to undergo a lytic cycle or never have undergone a lytic cycle. Functional studies of the full repertoire of prophages of bacterial genomes suggest that the majority of prophages are defective at some level: excision, virion formation, lysis, or infective ability (Bobay et al., 2014). Defective or cryptic prophages accrue to a high level of abundancy in many bacteria as a result of mutational decay and/or the loss of one or more genes essential to the lytic cycle over thousands of bacterial replication cycles. (Bobay et al., Pervasive domestication of defective prohages by bacteria, Proc Natl Acad Sci USA). Of note, defective prophages often also contain a number of genes that can provide adaptive or advantageous functionality to the host, including genes encoding proteins with homologous recombination functions, mechanisms for prevention of further infection, or bacteriocins, which may be helpful in competition for nutrients, e.g., through growth inhibition of other neighboring bacterial species. For example, several defective prophages have been characterized in E. coli K-12 (e.g., Rac, e14, DLP12, and QIN) and in Bacillus subtilis (e.g., 186 and SKIN) (Casjens, 2001, and references therein). Each of these phage harbors some functional genes. For example, Rac encodes the RecE homologous recombination system.

Accordingly, in some embodiments, the bacteria comprise one or more defective or cryptic prophages. In some embodiments, the prophage genes confer homologous recombination functions. In some embodiments, the prophage genes confer the ability to prevent further infection. In some embodiments, the prophage genes confer bacteriocins. IN some embodiments, the phage genes promote growth under adverse conditions by increasing carbon utilization, improving resistance to osmotic, oxidative and acid stresses, for increasing growth under various conditions, enhancing phosphorus and nitrogen utilization, or influencing biofilm formation.

In some embodiments, the bacteria comprise one or more satellite phage genomes. Satellite phages are otherwise functional phages that do not carry their own structural protein genes, and have genomes that are configures for encapsulation by the structural proteins of other specific phages (Six and Klug Bacteriophage P4: a satellite virus depending on a helper such as prophage P2, Virology, Volume 51, Issue 2, February 1973, Pages 327-344). Accordingly, in some embodiments, the bacteria comprise phage genomes which do not carry their own structural genes.

In some embodiments, the bacteria comprise one or more tailiocins. Many bacteria, both gram positive and gram negative, produce a variety of particles resembling phage tails that are functional without an associated phage head (termed tailiocins), and many of which have been shown to have bacteriocin properties (reviewed in Ghequire and Mot, The Tailocin Tale: Peeling off Phage; Trends in Microbiology, October 2015, Vol. 23, No. 10). Phage tail-like bacteriocins are classified two different families: contractile phage tail-like (R-type) and noncontractile but flexible ones (F-type). Accordingly, in some embodiments, bacteria comprise one or more tailiocins which confer bacteriocin or other beneficial properties.

In some embodiments, the bacteria comprise one or more gene transfer agents. Gene transfer agents (GTAs) are phage-like elements that are encoded by some bacterial genomes. Although GTAs resemble phages, they lack the hallmark capabilities that define typical phages, and they package random fragments of the host cell DNA and then transfer them horizontally to other bacteria of the same species (reviewed in Lang et al., Gene transfer agents: phage-like elements of genetic exchange, Nat Rev Microbiol. 2012 Jun. 11; 10(7): 472-482). There, the DNA can replace the resident cognate chromosomal region by homologous recombination. However, these particles cannot propagate as viruses, as the vast majority of the particles do not carry the genes that encode the GTA.

In some embodiments, the bacteria comprise one or more filamentous virions. Filamentous virions integrate as dsDNA prophages (reviewed in Marvin D A, et al, Structure and assembly of filamentous bacteriophages, Prog Biophys Mol Biol. 2014 April; 114(2):80-122).

In any of the embodiments described herein, the genetically engineered bacteria described herein which express one or more enzymes and transporters (e.g. for the consumption of phenylalanine), comprise one or more modifications or mutations within an endogenous prophage or bacteriophage genome. These modifications may alter the properties or behavior of the prophage. In some embodiments, the modifications or mutations essentially have no effect on bacterial fitness, and the bacterial fitness is essentially the same as the fitness of the isogenic strain without the modifications or mutations. Prophages can be either identified experimentally or computationally. The experimental approach involves inducing the host bacteria to release phage particles by exposing them to UV light or other DNA-damaging conditions. However, in some cases, the conditions under which a prophage is induced is unknown, and therefore the absence of plaques in a plaque assay does not necessarily prove the absence of a prophage. Additionally, this approach can show only the existence of viable phages, but will not reveal defective prophages. As such, computational identification of prophages from genomic sequence data has become the most preferred route.

In some embodiments, the modifications or mutations essentially have no effect on effector function, and the effector function is essentially the same as the effector function of the isogenic strain without the modifications or mutations. Table H provides a list of non-limiting examples of probiotic bacteria and the number of potential bacteriophages contained in the bacterial genome as determined by Phaster scoring. Table I provides a list of Clostridial strains and potential phage genomes. Phaster is a web server for bioinformatically identifying Phage sequences in organisms (http://phaster.ca/). Phaster scoring is described in detail at phaster.ca and in Zhou, et al. (“PHAST: A Fast Phage Search Tool” Nucl. Acids Res. (2011) 39(suppl 2): W347-W352) and Arndt et al. (Arndt, et al. (2016) PHASTER: a better, faster version of the PHAST phage search tool. Nucleic Acids Res., 2016 May 3). In brief, three methods are applied with different criteria to score for prophage regions (as intact, questionable, or incomplete) within a provided bacterial genome sequence. In the first method, if the number of certain phage organism identified by Phaster is more than or equal to 100% of the total number of CDS of the region, the region is marked with total score 150. If less than 100%, method 2 and 3 is used. In method 2, if the number of certain phage organism identified by Phaster in the bacterial genome sequence provided is more than 50% of the total number of CDS of the region, that phage organism is considered as the major potential phage for that region; the percentage of the total number of that phage organism in this table in the total number of proteins of the region is calculated and then multiplied by 100; the percentage of the length of that phage organism in the length of the region is calculated and then multiplied by 50 (phage head's encapsulation capability is considered). In method 3, if any of the specific phage-related keywords (such as ‘capsid’, ‘head’, ‘integrase’, ‘plate’, ‘tail’, ‘fiber’, ‘coat’, ‘transposase’, ‘portal’, ‘terminase’, ‘protease’ or ‘lysin’) are present, the score is increased by 10 for each keyword found. If the size of the region is greater than 30 Kb, the score is increased by 10. If there are at least 40 proteins in the region, the score is increased by 10. If all of the phage-related proteins and hypothetical proteins constitute more than 70% of the total number of proteins in the region, the score is increased by 10. The total score of method 2 is compared with the total score of method 3, and the bigger one is chosen as the total score of the region. If the region's total score is less than 70, it is marked as incomplete; if between 70 to 90, it is marked as questionable; if greater than 90, it is marked as intact.

TABLE H
Matched Strains for Common Probiotics

| | | PHASTER | |
|---|---|---|---|
| | PHASTER | questionable/ | |
| | Prophage | incomplete | ACLAME |
| Organism | (Intact) | (scores) | Prediction |
| Bacillus coagulans | | | |
| Bacillus subtilis | | | |
| Bacillus cereus | | | |
| Bifidobacterium animalis | 0 | 0 | |
| Bifidobacterium bifidum | 0 | 2 (90, 30) | |
| Bifidobacterium breve | 1 | 0 | |
| Bifidobacterium infantis | | | |
| Bifidobacterium longum | 0 | 1 (70) | |
| Enterococcus faecium | | | |
| Enterococcus durans | | | |
| Lactobacillus caucasicus | | | |
| Lactobacillus acidophilus | 0 | 1 (20) | |
| Lactobacillus brevis | 1 | 1 (20) | 2 |
| Lactobacillus casei | | | 2 |
| Lactobacillus delbrueckii | 0 | 0 | |
| Lactobacillus fermentum | 1 | 1 (40) | |
| Lactobacillus gasseri | 1 | 2 (60, 40) | |
| Lactobacillus helveticus | | | |
| Lactobacillus paracasei | 2 | 1 (30) | |
| Lactobacillus plantarum | 2 | 0 | 3 |
| Lactobacillus reuteri | 3 | 4 (70, 60, 40, 30) | 4 |
| Lactobacillus rhamnosus | 2 | 3 (70, 60, 40) | |
| Lactobacillus salivarius | 2 | 2 (50, 20) | 3 |
| Lactobacillus thermophilus | | | |
| Lactococcus lactis | 6 | 0 | 5 |
| Leuconostoc mesenteroides | | | |
| Pediococcus acidilactici | | | |
| Streptococcus thermophilus | | | |

TABLE I
Clostridial Strains

| | Intact | Incomplete | Questionable |
|---|---|---|---|
| | (phaster | (phaster | (phaster |
| | score) | score) | score) |
| Clostridium butyricum | 1 (110) | 3 (40, 40, 20) | 2 (90, 70) |
| 5521 | | | |
| Clostridium butyricum | 4 (150, 110, | 2 (50, 10) | 1 (70) |
| E4 str. BoNT E BL5262 | 130, 130) | | |
| Clostridium tyrobutyricum | | | |
| UC7086 | | | |
| Clostridium butyricum | 2 | | |
| strain KNU-L09 | | | |
| chromosome 1 | | | |
| Clostridium butyricum | 2 | | |
| strain CDC_51208 | | | |
| Clostridium butyricum | 2 | | |
| strain JKY6D1 | | | |
| chromosome 1 | | | |
| Clostridium butyricum | 1 | | |
| strain JKY6D1 | | | |
| chromosome 2 | | | |
| Clostridium tyrobutyricum | 5 | | |
| strain KCTC 5387 | | | |
| Clostridium butyricum | 2 | | |
| strain TOA | | | |
| chromosome 1 | | | |
| Clostridium butyricum | 1 | | |
| strain TOA | | | |
| chromosome 2 | | | |

In any of these embodiments, the bacteria described herein comprise one or more modifications or mutations within an existing prophage or bacteriophage genome. These modifications alter the properties or behavior of the prophage. In some embodiments, the modifications or mutations prevent the prophage from entering or completing the lytic process. In some embodiments, the modifications or mutations prevent the phage from infecting other bacteria of the same or a different type.

In some embodiments, the modifications or mutations alter, e.g., reduce or increase, the fitness of the bacterial host. In some embodiments, the modifications or mutations alter, e.g., reduce or increase, desired effector function, e.g., of a genetically engineered bacterium. In some embodiments, the modifications or mutations do not alter, e.g., reduce or increase, the fitness of the bacterial host. In some embodiments, the modifications or mutations do not alter, e.g., reduce or increase, desired effector function, e.g., of a genetically engineered bacterium.

In some embodiments, the modifications or mutations improve phenylalanine consumption. In some embodiments, phenylalanine consumption remains similar to the levels observed in the isogenic strain comprising the unmodified phage. In some embodiments, the modifications or mutations essentially have no effect on bacterial fitness, and the bacterial fitness is essentially the same as the fitness of the isogenic strain without the modifications or mutations.

In some embodiments, the bacteria comprise at least about 1 to 2, at least about 2 to 3, at least about 3 to 4, at least about 4 to 5, at least about 5 to 6, at least about 6 to 7, at least about 7 to 8, at least about 8 to 9, at least about 9 to 10, at least about 10 to 11, at least about 11 to 12, at least about 12 to 13, at least about 13 to 14, at least about 14 to 15, at least about 15 to 16, at least about 16 to 17, at least about 17 to 18, at least about 18 to 19, at least about 19 to 20, at least about 20 to 21, at least about 21 to 22, at least about 22 to 23, at least about 23 to 24, at least about 24 to 25, at least about 25 to 26, at least about 26 to 27, at least about 27 to 28, at least about 28 to 29, at least about 29 to 30, at least about 30 to 31, at least about 31 to 32, at least about 32 to 33, at least about 33 to 34, at least about 34 to 35, at least about 35 to 36, at least about 36 to 37, at least about 37 to 38, at least about 38 to 39, at least about 39 to 40, at least about 40 to 41, at least about 41 to 42, at least about 42 to 43, at least about 43 to 44, at least about 44 to 45, at least about 45 to 46, at least about 46 to 47, at least about 47 to 48, at least about 48 to 49, at least about 49 to 50, at least about 50 to 51, at least about 51 to 52, at least about 52 to 53, at least about 53 to 54, at least about 54 to 55, at least about 55 to 56, at least about 56 to 57, at least about 57 to 58, at least about 58 to 59, at least about 59 to 60, at least about 60 to 61, at least about 61 to 62, at least about 62 to 63, at least about 63 to 64, at least about 64 to 65, at least about 65 to 66, at least about 66 to 67, at least about 67 to 68, at least about 68 to 69, at least about 69 to 70, at least about 70 to 71, at least about 71 to 72, at least about 72 to 73, at least about 73 to 74, at least about 74 to 75, at least about 75 to 76, at least about 76 to 77, at least about 77 to 78, at least about 78 to 79, at least about 79 to 80, at least about 80 to 81, at least about 81 to 82, at least about 82 to 83, at least about 83 to 84, at least about 84 to 85, at least about 85 to 86, at least about 86 to 87, at least about 87 to 88, at least about 88 to 89, at least about 89 to 90, at least about 90 to 91, at least about 91 to 92, at least about 92 to 93, at least about 93 to 94, at least about 94 to 95, at least about 95 to 96, at least about 96 to 97, at least about 97 to 98, at least about 98 to 99, at least about 99 to 100, or at least about 100 or more modifications or mutations to an existing prophage or bacteriophage genome.

In some embodiments, the modifications or mutations improve effector function, e.g., phenylalanine consumption. In some embodiments, effector function, e.g., phenylalanine consumption, remains similar to that observed in the isogenic strain comprising the unmodified phage. In some embodiments, the modifications or mutations essentially have no effect on bacterial fitness, and the bacterial fitness is essentially the same as the fitness of the isogenic strain without the modifications or mutations.

In some embodiments, the modifications or mutations reduce entry or completion of prophage lytic process at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold relative to the isogenic strain without the phage modification. In some embodiments, the modifications or mutations completely prevent entry or completion of prophage lytic process.

In some embodiments, the modifications or mutations reduce entry or completion of prophage lytic process by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100% relative to the isogenic strain without the phage modification.

In some embodiments, the modifications or mutations prevent the phage from infecting other bacteria of the same or a different type by at least about 1- to 2-fold, at least about 2- to 3-fold, at least about 3- to 4-fold, at least about 4- to 5-fold, at least about 5- to 10-fold, at least about 10- to 100-fold, at least about 10- to 20-fold, at least about 20- to 30-fold, at least about 30- to 40-fold, at least about 40- to 50-fold, at least about 50- to 60-fold, at least about 60- to 70-fold, at least about 70- to 80-fold, at least about 80- to 90-fold, at least about 90- to 100-fold, or at least about 100- to 1000-fold relative to the isogenic strain without the phage modification. In some embodiments, the modifications or mutations completely prevent the phage from infecting other bacteria of the same or a different type. In some embodiments, the modifications or mutations prevent the phage from infecting other bacteria of the same or a different type by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100%.

In some embodiments, the modifications or mutations alters or alters, e.g., reduces or increases, the fitness of the bacterial host by at least about 1- to 2-fold, at least about 2- to 3-fold, at least about 3- to 4-fold, at least about 4- to 5-fold, at least about 5- to 10-fold, at least about 10- to 100-fold, or at least about 100- to 1000-fold relative to the isogenic strain without the phage modification. In some embodiments, the modifications or mutations alters, e.g., reduces or increases, the fitness of the bacterial host by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100% relative to the isogenic strain without the phage modification as compared to the isogenic strain without the phage modification.

In some embodiments, the modifications or mutations alter, e.g., reduce or increase, the desired effector function, e.g., of a genetically engineered bacterium by at least about 1- to 2-fold, at least about 2- to 3-fold, at least about 3- to 4-fold, at least about 4- to 5-fold, at least about 5- to 10-fold, at least about 10- to 100-fold, or at least about 100- to 1000-fold. In some embodiments, the modifications or mutations alter, e.g., reduce or increase, the desired effector function, e.g., of a genetically engineered bacterium by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100% relative to the isogenic strain without the phage modification.

In some embodiments, the mutations include one or more deletions within the phage genome sequence. As used herein, “deletion” refers to the removal of one or more nucleotides from a polynucleotide sequence. In some embodiments, the mutations include one or more insertions into the phage genome sequence. As used herein, “insertion” refers to the addition of one or more nucleotides to a polynucleotide sequence. In some embodiments, an antibiotic cassette can be inserted into one or more positions within the phage genome sequence. In some embodiments, the mutations include one or more substitutions within the phage genome sequence. As used herein, “substitution” refers to the replacement of one or more nucleotides with the same number of nucleotides within a polynucleotide sequence. In some embodiments, the mutations include one or more inversions within the phage genome sequence. As used herein, “inversion” refers to when a segment comprising 2 or more nucleotides is reversed end to end within a polynucleotide sequence. In some embodiments, the inversion may be governed by a specific flippase. Exemplary circuitry comprising multiple levels of control are exemplified herein and are also described in co-owned pending PCT Application PCT/US2016/039434, the contents of which is herein incorporated by reference in its entirety.

In some embodiments, the modifications within the phage genome are combinations of two or more of insertions, deletions, substitutions, or inversions within one or more phage genome genes.

In any of the embodiments described herein, the modifications may result in one or more frameshift mutations in one or more genes within the phage genome. As used herein, a frameshift mutation (also called a framing error or a reading frame shift) refers to a genetic mutation caused by indels (insertions or deletions) of a number of nucleotides in a DNA sequence that is not divisible by three. The earlier in the sequence the deletion or insertion occurs, the more altered the protein. In any of the embodiments described herein, the modifications may result in one or more missense mutation in one or more genes within the phage genome. As used herein, a missense mutation refers to when the change of a single base pair causes the substitution of a different amino acid in the resulting protein. This amino acid substitution may have no effect, or it may render the protein nonfunctional. In any of the embodiments described herein, the modifications may result in one or more nonsense mutations in one or more genes within the phage genome. As used herein, a nonsense mutation refers to a mutation in which a sense codon that corresponds to one of the twenty amino acids specified by the genetic code is changed to a chain-terminating codon and the polypeptide of interest is thereby truncated.

In some embodiments, the modifications within the phage genome are combinations of two or more frameshift, nonsense or missense mutations within one or more phage genome genes. In some embodiments, the bacteriophage that is modified is located on a bacterial chromosome. In some embodiments, the bacteriophage that is modified is located on a bacterial plasmid. In some embodiments, the plasmid is modified. In some embodiments, the plasmid is removed entirely. In some embodiments, the phage or prophage exists in all isolates of a particular species. In some embodiments, the prophage exists in all isolates of a particular phylum, order, sub order, family, class, subclass genus, species, sub species, or clade.

### Mutations

In some embodiments, the one or more mutations comprise at least about 1-500 bp of the phage genome. In some embodiments, the one or more mutations comprise at least about 500-1000 bp of the phage genome. In some embodiments, the one or more mutations comprise at least about 1000-2000 bp of the phage genome. In some embodiments, the one or more mutations comprise at least about 1000-2000 bp of the phage genome. In some embodiments, the one or more mutations comprise at least about 2000-3000 bp of the phage genome. In some embodiments, the one or more mutations comprise at least about 3000-4000 bp of the phage genome. In some embodiments, the one or more mutations comprise at least about 4000-5000 bp of the phage genome. In some embodiments, the one or more mutations comprise at least about 5,000-6,000 bp of the phage genome. In some embodiments, the one or more mutations comprise at least about 6,000-7,000 bp of the phage genome. In some embodiments, the one or more mutations comprise at least about 7,000-8,000 bp of the phage genome. In some embodiments, the one or more mutations comprise at least about 8,000-9,000 bp of the phage genome. In some embodiments, the one or more mutations comprise at least about 9,000-10,000 bp of the phage genome. In some embodiments, the one or more mutations comprise at least about 10,000-15,000 bp of the phage genome. In some embodiments, the one or more mutations comprise at least about 10,000-15,000 bp of the phage genome, at least about 15,000-20,000 bp of the phage genome, at least about 20,000-25,000 bp of the phage genome, at least about 25,000-30,000 bp of the phage genome, at least about 30,000-35,000 bp of the phage genome, at least about 35,000-40,000 bp of the phage genome, at least about 40,000-45,000 bp of the phage genome, at least about 45,000-50,000 bp of the phage genome, at least about 50,000-55,000 bp of the phage genome, at least about 55,000-60,000 bp of the phage genome, at least about 60,000-65,000 bp of the phage genome, at least about 65,000-70,000 bp of the phage genome, at least about 70,000-75,000 bp of the phage genome, at least about 75,000-80,000 bp of the phage genome, at least about 80,000-85,000 bp of the phage genome, at least about 85,000-90,000 bp of the phage genome, at least about 90,000-95,000 bp of the phage genome, at least about 95,000-100,000 bp of the phage genome, at least about 100,000-110,000 bp of the phage genome, at least about 110,000-120,000 bp of the phage genome, at least about 120,000-130,000 bp of the phage genome, at least about 130,000-140,000 bp of the phage genome, at least about 140,000-150,000 bp of the phage genome, at least about 150,000-200,000 bp of the phage genome, or more than at least about 200,000 bp of the phage genome. In one specific embodiment, 9687 bp of the phage genome are mutated. In some embodiments, the mutated nucleotides are interspersed. In some embodiments, the mutated nucleotides are consecutive. In some embodiments, at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the phage genome is mutated. In some embodiments, at least about 30-40% of the phage genome is mutated. In some embodiments, at least about 40-50% of the phage genome is mutated. In some embodiments, at least about 50-60% of the phage genome is mutated. In some embodiments, at least about 60-70% of the phage genome is mutated. In some embodiments, at least about 70-80% of the phage genome is mutated. In some embodiments, at least about 80-90% of the phage genome is mutated. In some embodiments, at least about 90-100% of the phage genome is mutated.

In some embodiments, at least about 1, 2, 3, 4, 5, 6, 7, 8, 9, 10, 11, 12, 13, 14, 15, 16, 17, 18, 19, or 20 genes are mutated. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes are mutated. In some embodiments, 13 genes are completely or partially mutated. In one embodiment, 74 genes are completely or partially mutated.

In some embodiments, at least about 1% to 2%, at least about 2% to 3%, at least about 3% to 4%, at least about 4% to 5%, at least about 5% to 6%, at least about 6% to 7%, at least about 7% to 8%, at least about 8% to 9%, at least about 9% to 10%, at least about 10% to 11%, at least about 11% to 12%, at least about 12% to 13%, at least about 13% to 14%, at least about 14% to 15%, at least about 15% to 16%, at least about 16% to 17%, at least about 17% to 18%, at least about 18% to 19%, at least about 19% to 20%, at least about 20% to 21%, at least about 21% to 22%, at least about 22% to 23%, at least about 23% to 24%, at least about 24% to 25%, at least about 25% to 26%, at least about 26% to 27%, at least about 27% to 28%, at least about 28% to 29%, at least about 29% to 30%, at least about 30% to 31%, at least about 31% to 32%, at least about 32% to 33%, at least about 33% to 34%, at least about 34% to 35%, at least about 35% to 36%, at least about 36% to 37%, at least about 37% to 38%, at least about 38% to 39%, at least about 39% to 40%, at least about 40% to 41%, at least about 41% to 42%, at least about 42% to 43%, at least about 43% to 44%, at least about 44% to 45%, at least about 45% to 46%, at least about 46% to 47%, at least about 47% to 48%, at least about 48% to 49%, at least about 49% to 50%, at least about 50% to 51%, at least about 51% to 52%, at least about 52% to 53%, at least about 53% to 54%, at least about 54% to 55%, at least about 55% to 56%, at least about 56% to 57%, at least about 57% to 58%, at least about 58% to 59%, at least about 59% to 60%, at least about 60% to 61%, at least about 61% to 62%, at least about 62% to 63%, at least about 63% to 64%, at least about 64% to 65%, at least about 65% to 66%, at least about 66% to 67%, at least about 67% to 68%, at least about 68% to 69%, at least about 69% to 70%, at least about 70% to 71%, at least about 71% to 72%, at least about 72% to 73%, at least about 73% to 74%, at least about 74% to 75%, at least about 75% to 76%, at least about 76% to 77%, at least about 77% to 78%, at least about 78% to 79%, at least about 79% to 80%, at least about 80% to 81%, at least about 81% to 82%, at least about 82% to 83%, at least about 83% to 84%, at least about 84% to 85%, at least about 85% to 86%, at least about 86% to 87%, at least about 87% to 88%, at least about 88% to 89%, at least about 89% to 90%, at least about 90% to 91%, at least about 91% to 92%, at least about 92% to 93%, at least about 93% to 94%, at least about 94% to 95%, at least about 95% to 96%, at least about 96% to 97%, at least about 97% to 98%, at least about 98% to 99%, at least about 99% to 100%, or at least about 100% of genes within the phage genome are completely or partially mutated.

In some embodiments, the one or more mutations are located at the beginning or 5′ end of the phage genome. In some embodiments, the one or more mutations are located at the end or 3′ end of the phage genome. In some embodiments, the one or more mutations are located in the middle of the phage genome. In some embodiments, the phage genes are interspersed within the bacterial genome and the mutation are located in one or more of the interspersed positions.

In some embodiments, the region for an optimal mutation, i.e., to achieve a desired effect, can be determined through analysis of homology with other phages in other bacteria. Homologous conserved regions in phages may be suitable for mutation, as these are conserved and may comprise one or more essential genes. In some embodiments, regulatory elements, such as promoters, are mutated. In some embodiments, coding sequences are mutated. In some embodiments, the one or more mutated regions contain one or more genes essential for the lytic cycle.

In some embodiments, the mutations are located within or encompass one or more genes encoding lytic genes. In some embodiments, the mutations are located within or encompass one or more genes encoding one or more proteases or lysins. In some embodiments, the mutations are located within or encompass one or more genes encoding one or more toxins. In some embodiments, the mutations are located within or encompass one or more genes encoding one or more antibiotic resistance related proteins. In some embodiments, the mutations are located within or encompass one or more genes encoding one or phage translation related proteins. In some embodiments, the one or more mutations are located within or encompass one or more genes encoding structural proteins. Such structural genes include genes encoding polypeptides of the head, tail, collar, or coat. In some embodiments, the one or more mutations are located within or encompass one or more genes encoding polypeptides of the head structure. In some embodiments, the one or more mutations are located within or encompass one or more genes encoding polypeptides of the tail structure. In some embodiments, the one or more mutations are located within or encompass one or more genes encoding polypeptides of the collar structure. In some embodiments, the one or more mutations are located within or encompass one or more genes encoding tail proteins. In some embodiments, the one or more mutations are located within or encompass one or more genes encoding polypeptides of the coat structure. In some embodiments, the mutations are located within or encompass one or more genes encoding one or more plate proteins. In some embodiments, the mutations are located within or encompass one or more genes encoding one or more proteins require for assembly of the bacteriophage. In some embodiments, the mutations are located within or encompass one or more genes encoding one or more portal proteins. In some embodiments, the mutations are located within or encompass one or more genes encoding one or more polypeptides involved in recombination. In some embodiments, the mutations are located within or encompass one or more genes encoding one or more integrases. In some embodiments, the mutations are located within or encompass one or more genes encoding one or more invertases. In some embodiments, the mutations are located within or encompass one or more genes encoding one or more transposases. In some embodiments, the mutations are located with within or encompass one or more genes encoding one or more polypeptides involved in replication or translation. In some embodiments, the mutations are located within or encompass one or more genes encoding one or more primases. In some embodiments, the mutations are located within or encompass one or more genes encoding one or more tRNA related proteins. In some embodiments, the mutations are located within or encompass one or more genes encoding one or more polypeptides involved in phage insertion. In some embodiments, the mutations are located within or encompass one or more genes encoding an attachment site. In some embodiments, the mutations are located within or encompass one or more genes encoding one or more polypeptides involved in packaging. In some embodiments, the mutations are located within or encompass one or more genes encoding one or more terminases. In some embodiments, the mutations are located within or encompass one or more genes encoding one or more host genes.

In some embodiments, the mutations are located within or encompass genes encoding one or more polypeptides involved in one or more of cell lysis, phage structure, phage assembly, phage packaging recombination, replication or translation, phage insertion, or are host proteins, and combinations thereof.

In some embodiments, the mutations are located within or encompass genes encoding one or more polypeptides involved in one or more of cell lysis, phage structure, phage assembly, phage packaging recombination, replication or translation, phage insertion, and combinations thereof.

In some embodiments, the mutations are located within or encompass 1 genes encoding polypeptides involved in cell lysis, phage structure, phage assembly, phage packaging recombination, replication or translation, phage insertion, and combinations thereof. In some embodiments, the mutations are located within or encompass 2 genes encoding polypeptides involved in cell lysis, phage structure, phage assembly, phage packaging recombination, replication or translation, phage insertion, and combinations thereof. In some embodiments, the mutations are located within or encompass 3 genes encoding polypeptides involved in cell lysis, phage structure, phage assembly, phage packaging recombination, replication or translation, phage insertion, and combinations thereof. In some embodiments, the mutations are located within or encompass 4 genes encoding polypeptides involved in cell lysis, phage structure, phage assembly, phage packaging recombination, replication or translation, phage insertion, and combinations thereof. In some embodiments, the mutations are located within or encompass 2 genes encoding polypeptides involved in cell lysis, phage structure, phage assembly, phage packaging recombination, replication or translation, phage insertion, and combinations thereof. In some embodiments, the mutations are located within or encompass 5 genes encoding polypeptides involved in cell lysis, phage structure, phage assembly, phage packaging recombination, replication or translation, phage insertion, and combinations thereof. In some embodiments, the mutations are located within or encompass 6 genes encoding polypeptides involved in cell lysis, phage structure, phage assembly, phage packaging recombination, replication or translation, phage insertion, and combinations thereof. In some embodiments, the mutations are located within or encompass 7 genes encoding polypeptides involved in cell lysis, phage structure, phage assembly, phage packaging recombination, replication or translation, phage insertion, and combinations thereof. In some embodiments, the mutations are located within or encompass 8 genes encoding polypeptides involved in cell lysis, phage structure, phage assembly, phage packaging recombination, replication or translation, phage insertion, and combinations thereof. In some embodiments, the mutations are located within or encompass 9 genes encoding polypeptides involved in cell lysis, phage structure, phage assembly, phage packaging recombination, replication or translation, phage insertion, and combinations thereof. In some embodiments, the mutations are located within or encompass 10 genes encoding polypeptides involved in cell lysis, phage structure, phage assembly, phage packaging recombination, replication or translation, phage insertion, and combinations thereof. In some embodiments, the mutations are located within or encompass 11 genes encoding polypeptides involved in cell lysis, phage structure, phage assembly, phage packaging recombination, replication or translation, phage insertion, and combinations thereof. In some embodiments, the mutations are located within or encompass 12 genes encoding polypeptides involved in cell lysis, phage structure, phage assembly, phage packaging recombination, replication or translation, phage insertion, and combinations thereof. In some embodiments, the mutations are located within or encompass 13 genes encoding polypeptides involved in cell lysis, phage structure, phage assembly, phage packaging recombination, replication or translation, phage insertion, and combinations thereof. In some embodiments, the mutations are located within or encompass 14 genes encoding polypeptides involved in cell lysis, phage structure, phage assembly, phage packaging recombination, replication or translation, phage insertion, and combinations thereof. In some embodiments, the mutations are located within or encompass 15 genes encoding polypeptides involved in cell lysis, phage structure, phage assembly, phage packaging recombination, replication or translation, phage insertion, and combinations thereof. In some embodiments, the mutations are located within or encompass at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the mutations are located within or encompass one or more host proteins within the phage genome.

### Deletions

In some embodiments, the one or more deletions comprise at least about 1-500 bp of the phage genome. In some embodiments, the one or more deletions comprise at least about 500-1000 bp of the phage genome. In some embodiments, the one or more deletions comprise at least about 1000-2000 bp of the phage genome. In some embodiments, the one or more deletions comprise at least about 1000-2000 bp of the phage genome. In some embodiments, the one or more deletions comprise at least about 2000-3000 bp of the phage genome. In some embodiments, the one or more deletions comprise at least about 3000-4000 bp of the phage genome. In some embodiments, the one or more deletions comprise at least about 4000-5000 bp of the phage genome. In some embodiments, the one or more deletions comprise at least about 5,000-6,000 bp of the phage genome. In some embodiments, the one or more deletions comprise at least about 6,000-7,000 bp of the phage genome. In some embodiments, the one or more deletions comprise at least about 7,000-8,000 bp of the phage genome. In some embodiments, the one or more deletions comprise at least about 8,000-9,000 bp of the phage genome. In some embodiments, the one or more deletions comprise at least about 9,000-10,000 bp of the phage genome. In some embodiments, the one or more deletions comprise at least about 10,000-15,000 bp of the phage genome. In some embodiments, the one or more deletions comprise at least about 10,000-15,000 bp of the phage genome, at least about 15,000-20,000 bp of the phage genome, at least about 20,000-25,000 bp of the phage genome, at least about 25,000-30,000 bp of the phage genome, at least about 30,000-35,000 bp of the phage genome, at least about 35,000-40,000 bp of the phage genome, at least about 40,000-45,000 bp of the phage genome, at least about 45,000-50,000 bp of the phage genome, at least about 50,000-55,000 bp of the phage genome, at least about 55,000-60,000 bp of the phage genome, at least about 60,000-65,000 bp of the phage genome, at least about 65,000-70,000 bp of the phage genome, at least about 70,000-75,000 bp of the phage genome, at least about 75,000-80,000 bp of the phage genome, at least about 80,000-85,000 bp of the phage genome, at least about 85,000-90,000 bp of the phage genome, at least about 90,000-95,000 bp of the phage genome, at least about 95,000-100,000 bp of the phage genome, at least about 100,000-110,000 bp of the phage genome, at least about 110,000-120,000 bp of the phage genome, at least about 120,000-130,000 bp of the phage genome, at least about 130,000-140,000 bp of the phage genome, at least about 140,000-150,000 bp of the phage genome, at least about 150,000-200,000 bp of the phage genome, or more than 200,000 bp of the phage genome. In one specific embodiment, 9687 bp of the phage genome are deleted. In some embodiments, the deleted nucleotides are interspersed. In some embodiments, the deleted nucleotides are consecutive.

In some embodiments, at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the phage genome is deleted. In some embodiments, at least about 30-40% of the phage genome is deleted. In some embodiments, at least about 40-50% of the phage genome is deleted. In some embodiments, at least about 50-60% of the phage genome is deleted. In some embodiments, at least about 60-70% of the phage genome is deleted. In some embodiments, at least about 70-80% of the phage genome is deleted. In some embodiments, at least about 80-90% of the phage genome is deleted. In some embodiments, at least about 90-100% of the phage genome is deleted.

In some embodiments, one or more genes are partially or completely deleted within the phage genome. In some embodiments, one or more genes are completely deleted and one or more genes are partially deleted. In one embodiment, there is one deletion within the phage genome and one or two genes at the ends of the deletion are partially deleted and the rest of the genes are completely deleted. In some embodiments, the deleted genes are adjacent to each other. In some embodiments, the deleted genes are not adjacent to each other.

In some embodiments, at least about 1, 2, 3, 4, 5, 6, 7, 8, 9, 10, 11, 12, 13, 14, 15, 16, 17, 18, 19, or 20 genes are deleted. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes are deleted. In some embodiments, 13 genes are completely or partially deleted. In one embodiment, 74 genes are completely or partially deleted. In some embodiments, at least about 1% to 2%, at least about 2% to 3%, at least about 3% to 4%, at least about 4% to 5%, at least about 5% to 6%, at least about 6% to 7%, at least about 7% to 8%, at least about 8% to 9%, at least about 9% to 10%, at least about 10% to 11%, at least about 11% to 12%, at least about 12% to 13%, at least about 13% to 14%, at least about 14% to 15%, at least about 15% to 16%, at least about 16% to 17%, at least about 17% to 18%, at least about 18% to 19%, at least about 19% to 20%, at least about 20% to 21%, at least about 21% to 22%, at least about 22% to 23%, at least about 23% to 24%, at least about 24% to 25%, at least about 25% to 26%, at least about 26% to 27%, at least about 27% to 28%, at least about 28% to 29%, at least about 29% to 30%, at least about 30% to 31%, at least about 31% to 32%, at least about 32% to 33%, at least about 33% to 34%, at least about 34% to 35%, at least about 35% to 36%, at least about 36% to 37%, at least about 37% to 38%, at least about 38% to 39%, at least about 39% to 40%, at least about 40% to 41%, at least about 41% to 42%, at least about 42% to 43%, at least about 43% to 44%, at least about 44% to 45%, at least about 45% to 46%, at least about 46% to 47%, at least about 47% to 48%, at least about 48% to 49%, at least about 49% to 50%, at least about 50% to 51%, at least about 51% to 52%, at least about 52% to 53%, at least about 53% to 54%, at least about 54% to 55%, at least about 55% to 56%, at least about 56% to 57%, at least about 57% to 58%, at least about 58% to 59%, at least about 59% to 60%, at least about 60% to 61%, at least about 61% to 62%, at least about 62% to 63%, at least about 63% to 64%, at least about 64% to 65%, at least about 65% to 66%, at least about 66% to 67%, at least about 67% to 68%, at least about 68% to 69%, at least about 69% to 70%, at least about 70% to 71%, at least about 71% to 72%, at least about 72% to 73%, at least about 73% to 74%, at least about 74% to 75%, at least about 75% to 76%, at least about 76% to 77%, at least about 77% to 78%, at least about 78% to 79%, at least about 79% to 80%, at least about 80% to 81%, at least about 81% to 82%, at least about 82% to 83%, at least about 83% to 84%, at least about 84% to 85%, at least about 85% to 86%, at least about 86% to 87%, at least about 87% to 88%, at least about 88% to 89%, at least about 89% to 90%, at least about 90% to 91%, at least about 91% to 92%, at least about 92% to 93%, at least about 93% to 94%, at least about 94% to 95%, at least about 95% to 96%, at least about 96% to 97%, at least about 97% to 98%, at least about 98% to 99%, at least about 99% to 100%, or at least about 100% of genes within the phage genome are completely or partially deleted.

In some embodiments, the one or more deletions are located at the beginning or 5′ end of the phage genome. In some embodiments, the one or more deletions are located at the end or 3′ end of the phage genome. In some embodiments, the one or more deletions are located in the middle of the phage genome. In some embodiments, the phage genes are interspersed within the bacterial genome and the deletion are located in one or more of the interspersed positions.

In some embodiments, the region for an optimal deletion, i.e., to achieve a desired effect, can be determined through analysis of homology with other phages is other bacteria. Homologous conserved regions in phages may be suitable for deletion, as these are conserved and may comprise one or more essential genes. In some embodiments, regulatory elements, such as promoters, are deleted. In some embodiments, coding sequences are deleted. In some embodiments, the one or more deleted regions contain one or more genes essential for the lytic cycle.

In some embodiments, the deletions are located within or encompass one or more genes encoding lytic genes. In some embodiments, the deletions are located within or encompass one or more genes encoding one or more proteases or lysins. In some embodiments, the deletions are located within or encompass one or more genes encoding one or more toxins. In some embodiments, the deletions are located within or encompass one or more genes encoding one or more antibiotic resistance related proteins. In some embodiments, the deletions are located within or encompass one or more genes encoding one or phage translation related proteins. In some embodiments, the one or more deletions are located within or encompass one or more genes encoding structural proteins. Such structural genes include genes encoding polypeptides of the head, tail, collar, or coat. In some embodiments, the one or more deletions are located within or encompass one or more genes encoding polypeptides of the head structure. In some embodiments, the one or more deletions are located within or encompass one or more genes encoding polypeptides of the tail structure. In some embodiments, the one or more deletions are located within or encompass one or more genes encoding polypeptides of the collar structure. In some embodiments, the one or more deletions are located within or encompass one or more genes encoding polypeptides of the coat structure. In some embodiments, the deletions are located within or encompass one or more genes encoding one or more plate proteins. In some embodiments, the deletions are located within or encompass one or more genes encoding one or more proteins require for assembly of the bacteriophage. In some embodiments, the deletions are located within or encompass one or more genes encoding one or more portal proteins. In some embodiments, the deletions are located within or encompass one or more genes encoding one or more polypeptides involved in recombination. In some embodiments, the deletions are located within or encompass one or more genes encoding one or more integrases. In some embodiments, the deletions are located within or encompass one or more genes encoding one or more invertases. In some embodiments, the deletions are located within or encompass one or more genes encoding one or more transposases. In some embodiments, the deletions are located with within or encompass one or more genes encoding one or more polypeptides involved in replication or translation. In some embodiments, the deletions are located within or encompass one or more genes encoding one or more primases. In some embodiments, the deletions are located within or encompass one or more genes encoding one or more tRNA related proteins. In some embodiments, the deletions are located within or encompass one or more genes encoding one or more polypeptides involved in phage insertion. In some embodiments, the deletions are located within or encompass one or more genes encoding an attachment site. In some embodiments, the deletions are located within or encompass one or more genes encoding one or more polypeptides involved in packaging. In some embodiments, the deletions are located within or encompass one or more genes encoding one or more terminases. In some embodiments, the deletions are located within or encompass one or more genes encoding one or more host genes.

In some embodiments, the deletions are located within or encompass genes encoding one or more polypeptides involved in one or more of cell lysis, phage structure, phage assembly, phage packaging recombination, replication or translation, phage insertion, or are host proteins, and combinations thereof.

In some embodiments, the deletions are located within or encompass genes encoding one or more polypeptides involved in one or more of cell lysis, phage structure, phage assembly, phage packaging recombination, replication or translation, phage insertion, and combinations thereof.

In some embodiments, the deletions are located within or encompass 1 genes encoding polypeptides involved in cell lysis, phage structure, phage assembly, phage packaging recombination, replication or translation, phage insertion, and combinations thereof. In some embodiments, the deletions are located within or encompass 2 genes encoding polypeptides involved in cell lysis, phage structure, phage assembly, phage packaging recombination, replication or translation, phage insertion, and combinations thereof. In some embodiments, the deletions are located within or encompass 3 genes encoding polypeptides involved in cell lysis, phage structure, phage assembly, phage packaging recombination, replication or translation, phage insertion, and combinations thereof. In some embodiments, the deletions are located within or encompass 4 genes encoding polypeptides involved in cell lysis, phage structure, phage assembly, phage packaging recombination, replication or translation, phage insertion, and combinations thereof. In some embodiments, the deletions are located within or encompass 2 genes encoding polypeptides involved in cell lysis, phage structure, phage assembly, phage packaging recombination, replication or translation, phage insertion, and combinations thereof. In some embodiments, the deletions are located within or encompass 5 genes encoding polypeptides involved in cell lysis, phage structure, phage assembly, phage packaging recombination, replication or translation, phage insertion, and combinations thereof. In some embodiments, the deletions are located within or encompass 6 genes encoding polypeptides involved in cell lysis, phage structure, phage assembly, phage packaging recombination, replication or translation, phage insertion, and combinations thereof. In some embodiments, the deletions are located within or encompass 7 genes encoding polypeptides involved in cell lysis, phage structure, phage assembly, phage packaging recombination, replication or translation, phage insertion, and combinations thereof. In some embodiments, the deletions are located within or encompass 8 genes encoding polypeptides involved in cell lysis, phage structure, phage assembly, phage packaging recombination, replication or translation, phage insertion, and combinations thereof. In some embodiments, the deletions are located within or encompass 9 genes encoding polypeptides involved in cell lysis, phage structure, phage assembly, phage packaging recombination, replication or translation, phage insertion, and combinations thereof. In some embodiments, the deletions are located within or encompass 10 genes encoding polypeptides involved in cell lysis, phage structure, phage assembly, phage packaging recombination, replication or translation, phage insertion, and combinations thereof. In some embodiments, the deletions are located within or encompass 11 genes encoding polypeptides involved in cell lysis, phage structure, phage assembly, phage packaging recombination, replication or translation, phage insertion, and combinations thereof. In some embodiments, the deletions are located within or encompass 12 genes encoding polypeptides involved in cell lysis, phage structure, phage assembly, phage packaging recombination, replication or translation, phage insertion, and combinations thereof. In some embodiments, the deletions are located within or encompass 13 genes encoding polypeptides involved in cell lysis, phage structure, phage assembly, phage packaging recombination, replication or translation, phage insertion, and combinations thereof. In some embodiments, the deletions are located within or encompass 14 genes encoding polypeptides involved in cell lysis, phage structure, phage assembly, phage packaging recombination, replication or translation, phage insertion, and combinations thereof. In some embodiments, the deletions are located within or encompass 15 genes encoding polypeptides involved in cell lysis, phage structure, phage assembly, phage packaging recombination, replication or translation, phage insertion, and combinations thereof. In some embodiments, the deletions are located within or encompass at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the deletions are located within or encompass one or more host proteins within the phage genome.

### Insertions

In some embodiments, the insertion is in a coding region of the phage genome. In some embodiments, the insertion is inserted into a regulatory region of the phage genome. In some embodiments, the insertions comprise one or more antibiotic cassette(s). suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more insertions comprise at least about 1-500 bp in length. In some embodiments, the one or more insertions comprise at least about 500-1000 bp in length. In some embodiments, the one or more insertions comprise at least about 1000-2000 bp in length. In some embodiments, the one or more insertions comprise at least about 1000-2000 bp in length. In some embodiments, the one or more insertions comprise at least about 2000-3000 bp in length. In some embodiments, the one or more insertions comprise at least about 3000-4000 bp in length. In some embodiments, the one or more insertions comprise at least about 4000-5000 bp in length. In some embodiments, the one or more insertions comprise at least about 5,000-6,000 bp in length. In some embodiments, the one or more insertions comprise at least about 6,000-7,000 bp in length. In some embodiments, the one or more insertions comprise at least about 7,000-8,000 bp in length. In some embodiments, the one or more insertions comprise at least about 8,000-9,000 bp in length. In some embodiments, the one or more insertions comprise at least about 9,000-10,000 bp in length. In some embodiments, the one or more insertions comprise at least about 10,000-15,000 bp in length. In some embodiments, the one or more insertions comprise at least about 10,000-15,000 bp in length, at least about 15,000-20,000 bp in length, at least about 20,000-25,000 bp in length, at least about 25,000-30,000 bp in length, at least about 30,000-35,000 bp in length, at least about 35,000-40,000 bp in length, at least about 40,000-45,000 bp in length, at least about 45,000-50,000 bp in length, at least about 50,000-55,000 bp in length, at least about 55,000-60,000 bp in length, at least about 60,000-65,000 bp in length, at least about 65,000-70,000 bp in length, at least about 70,000-75,000 bp in length, at least about 75,000-80,000 bp in length, at least about 80,000-85,000 bp in length, at least about 85,000-90,000 bp in length, at least about 90,000-95,000 bp in length, at least about 95,000-100,000 bp in length, at least about 100,000-110,000 bp in length, at least about 110,000-120,000 bp in length, at least about 120,000-130,000 bp in length, at least about 130,000-140,000 bp in length, at least about 140,000-150,000 bp in length, at least about 150,000-200,000 bp in length, or more than at least about 200,000 bp in length. In one specific embodiment, 9687 bp in length are inserted. In some embodiments, the inserted nucleotides are interspersed. In some embodiments, the inserted nucleotides are consecutive.

In some embodiments, the one or more insertions are located within 1-500 bp of the phage genome. In some embodiments, the one or more insertions are located within at least about 500-1000 bp of the phage genome. In some embodiments, the one or more insertions are located within at least about 1000-2000 bp of the phage genome. In some embodiments, the one or more insertions are located within at least about 1000-2000 bp of the phage genome. In some embodiments, the one or more insertions are located within at least about 2000-3000 bp of the phage genome. In some embodiments, the one or more insertions are located within at least about 3000-4000 bp of the phage genome. In some embodiments, the one or more insertions are located within at least about 4000-5000 bp of the phage genome. In some embodiments, the one or more insertions are located within at least about 5,000-6,000 bp of the phage genome. In some embodiments, the one or more insertions are located within at least about 6,000-7,000 bp of the phage genome. In some embodiments, the one or more insertions are located within at least about 7,000-8,000 bp of the phage genome. In some embodiments, the one or more insertions are located within at least about 8,000-9,000 bp of the phage genome. In some embodiments, the one or more insertions are located within at least about 9,000-10,000 bp of the phage genome. In some embodiments, the one or more insertions are located within at least about 10,000-15,000 bp of the phage genome. In some embodiments, the one or more insertions are located within at least about 10,000-15,000 bp of the phage genome, at least about 15,000-20,000 bp of the phage genome, at least about 20,000-25,000 bp of the phage genome, at least about 25,000-30,000 bp of the phage genome, at least about 30,000-35,000 bp of the phage genome, at least about 35,000-40,000 bp of the phage genome, at least about 40,000-45,000 bp of the phage genome, at least about 45,000-50,000 bp of the phage genome, at least about 50,000-55,000 bp of the phage genome, at least about 55,000-60,000 bp of the phage genome, at least about 60,000-65,000 bp of the phage genome, at least about 65,000-70,000 bp of the phage genome, at least about 70,000-75,000 bp of the phage genome, at least about 75,000-80,000 bp of the phage genome, at least about 80,000-85,000 bp of the phage genome, at least about 85,000-90,000 bp of the phage genome, at least about 90,000-95,000 bp of the phage genome, at least about 95,000-100,000 bp of the phage genome, at least about 100,000-110,000 bp of the phage genome, at least about 110,000-120,000 bp of the phage genome, at least about 120,000-130,000 bp of the phage genome, at least about 130,000-140,000 bp of the phage genome, at least about 140,000-150,000 bp of the phage genome, at least about 150,000-200,000 bp of the phage genome, or more than at least about 200,000 bp of the phage genome. In one specific embodiment, 9687 bp of the phage genome are inserted. In some embodiments, the inserted nucleotides are interspersed. In some embodiments, the inserted nucleotides are consecutive.

In some embodiments, the insertions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the phage genome. In some embodiments, at least about 30-40% of the phage genome is inserted. In some embodiments, the insertions are located within at least about 40-50% of the phage genome. In some embodiments, the insertions are located within at least about 50-60% of the phage genome. In some embodiments, the insertions are located within at least about 60-70% of the phage genome. In some embodiments, the insertions are located within at least about 70-80% of the phage genome. In some embodiments, the insertions are located within at least about 80-90% of the phage genome. In some embodiments, the insertions are located within at least about 90-100% of the phage genome.

In some embodiments, at least about 1, 2, 3, 4, 5, 6, 7, 8, 9, 10, 11, 12, 13, 14, 15, 16, 17, 18, 19, or 20 genes comprise insertions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise insertions. In some embodiments, 13 genes comprise insertions. In one embodiment, 74 genes comprise insertions.

In some embodiments, the one or more insertions are located at the beginning or 5′ end of the phage genome. In some embodiments, the one or more insertions are located at the end or 3′ end of the phage genome. In some embodiments, the one or more insertions are located in the middle of the phage genome. In some embodiments, the phage genes are interspersed within the bacterial genome and the insertion are located in one or more of the interspersed positions.

In some embodiments, the region for an optimal insertion, i.e., to achieve a desired effect, can be determined through analysis of homology with other phages is other bacteria. Homologous conserved regions in phages may be suitable for insertion, as these are conserved and may comprise one or more essential genes. In some embodiments, regulatory elements, such as promoters, are inserted. In some embodiments, coding sequences are inserted. In some embodiments, the one or more inserted regions contain one or more genes essential for the lytic cycle.

In some embodiments, the insertions are located within one or more genes encoding lytic genes. In some embodiments, the insertions are located within one or more genes encoding one or more proteases or lysins. In some embodiments, the insertions are located within one or more genes encoding one or more toxins. In some embodiments, the insertions are located within one or more genes encoding one or more antibiotic resistance related proteins. In some embodiments, the insertions are located within one or more genes encoding one or phage translation related proteins. In some embodiments, the one or more insertions are located within one or more genes encoding structural proteins. Such structural genes include genes encoding polypeptides of the head, tail, collar, or coat. In some embodiments, the one or more insertions are located within one or more genes encoding polypeptides of the head structure. In some embodiments, the one or more insertions are located within one or more genes encoding polypeptides of the tail structure. In some embodiments, the one or more insertions are located within one or more genes encoding polypeptides of the collar structure. In some embodiments, the one or more insertions are located within one or more genes encoding polypeptides of the coat structure. In some embodiments, the insertions are located within one or more genes encoding one or more plate proteins. In some embodiments, the insertions are located within one or more genes encoding one or more proteins require for assembly of the bacteriophage. In some embodiments, the insertions are located within one or more genes encoding one or more portal proteins. In some embodiments, the insertions are located within one or more genes encoding one or more polypeptides involved in recombination. In some embodiments, the insertions are located within one or more genes encoding one or more integrases. In some embodiments, the insertions are located within one or more genes encoding one or more invertases. In some embodiments, the insertions are located within one or more genes encoding one or more transposases. In some embodiments, the insertions are located with within one or more genes encoding one or more polypeptides involved in replication or translation. In some embodiments, the insertions are located within one or more genes encoding one or more primases. In some embodiments, the insertions are located within one or more genes encoding one or more tRNA related proteins. In some embodiments, the insertions are located within one or more genes encoding one or more polypeptides involved in phage insertion. In some embodiments, the insertions are located within one or more genes encoding an attachment site. In some embodiments, the insertions are located within one or more genes encoding one or more polypeptides involved in packaging. In some embodiments, the insertions are located within one or more genes encoding one or more terminases. In some embodiments, the insertions are located within one or more genes encoding one or more host genes.

In some embodiments, the insertions are located within genes encoding one or more polypeptides involved in one or more of cell lysis, phage structure, phage assembly, phage packaging recombination, replication or translation, phage insertion, or are host proteins, and combinations thereof.

In some embodiments, the insertions are located within genes encoding one or more polypeptides involved in one or more of cell lysis, phage structure, phage assembly, phage packaging recombination, replication or translation, phage insertion, and combinations thereof.

In some embodiments, the insertions are located within 1 genes encoding polypeptides involved in cell lysis, phage structure, phage assembly, phage packaging recombination, replication or translation, phage insertion, and combinations thereof. In some embodiments, the insertions are located within 2 genes encoding polypeptides involved in cell lysis, phage structure, phage assembly, phage packaging recombination, replication or translation, phage insertion, and combinations thereof. In some embodiments, the insertions are located within 3 genes encoding polypeptides involved in cell lysis, phage structure, phage assembly, phage packaging recombination, replication or translation, phage insertion, and combinations thereof. In some embodiments, the insertions are located within 4 genes encoding polypeptides involved in cell lysis, phage structure, phage assembly, phage packaging recombination, replication or translation, phage insertion, and combinations thereof. In some embodiments, the insertions are located within 2 genes encoding polypeptides involved in cell lysis, phage structure, phage assembly, phage packaging recombination, replication or translation, phage insertion, and combinations thereof. In some embodiments, the insertions are located within 5 genes encoding polypeptides involved in cell lysis, phage structure, phage assembly, phage packaging recombination, replication or translation, phage insertion, and combinations thereof. In some embodiments, the insertions are located within 6 genes encoding polypeptides involved in cell lysis, phage structure, phage assembly, phage packaging recombination, replication or translation, phage insertion, and combinations thereof. In some embodiments, the insertions are located within 7 genes encoding polypeptides involved in cell lysis, phage structure, phage assembly, phage packaging recombination, replication or translation, phage insertion, and combinations thereof. In some embodiments, the insertions are located within 8 genes encoding polypeptides involved in cell lysis, phage structure, phage assembly, phage packaging recombination, replication or translation, phage insertion, and combinations thereof. In some embodiments, the insertions are located within 9 genes encoding polypeptides involved in cell lysis, phage structure, phage assembly, phage packaging recombination, replication or translation, phage insertion, and combinations thereof. In some embodiments, the insertions are located within 10 genes encoding polypeptides involved in cell lysis, phage structure, phage assembly, phage packaging recombination, replication or translation, phage insertion, and combinations thereof. In some embodiments, the insertions are located within 11 genes encoding polypeptides involved in cell lysis, phage structure, phage assembly, phage packaging recombination, replication or translation, phage insertion, and combinations thereof. In some embodiments, the insertions are located within 12 genes encoding polypeptides involved in cell lysis, phage structure, phage assembly, phage packaging recombination, replication or translation, phage insertion, and combinations thereof. In some embodiments, the insertions are located within 13 genes encoding polypeptides involved in cell lysis, phage structure, phage assembly, phage packaging recombination, replication or translation, phage insertion, and combinations thereof. In some embodiments, the insertions are located within 14 genes encoding polypeptides involved in cell lysis, phage structure, phage assembly, phage packaging recombination, replication or translation, phage insertion, and combinations thereof. In some embodiments, the insertions are located within 15 genes encoding polypeptides involved in cell lysis, phage structure, phage assembly, phage packaging recombination, replication or translation, phage insertion, and combinations thereof. In some embodiments, the insertions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the insertions are located within one or more host proteins within the phage genome.

### Inversions

In some embodiments, the inversion is in a coding region of the phage genome. In some embodiments, the inversion is inverted into a regulatory region of the phage genome. In some embodiments, the inversions comprise one or more antibiotic cassette(s). suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more inversions comprise at least about 1-500 bp. In some embodiments, the one or more inversions comprise at least about 500-1000 bp. In some embodiments, the one or more inversions comprise at least about 1000-2000 bp. In some embodiments, the one or more inversions comprise at least about 1000-2000 bp. In some embodiments, the one or more inversions comprise at least about 2000-3000 bp. In some embodiments, the one or more inversions comprise at least about 3000-4000 bp. In some embodiments, the one or more inversions comprise at least about 4000-5000 bp. In some embodiments, the one or more inversions comprise at least about 5,000-6,000 bp. In some embodiments, the one or more inversions comprise at least about 6,000-7,000 bp. In some embodiments, the one or more inversions comprise at least about 7,000-8,000 bp. In some embodiments, the one or more inversions comprise at least about 8,000-9,000 bp. In some embodiments, the one or more inversions comprise at least about 9,000-10,000 bp. In some embodiments, the one or more inversions comprise at least about 10,000-15,000 bp. In some embodiments, the one or more inversions comprise at least about 10,000-15,000 bp, at least about 15,000-20,000 bp, at least about 20,000-25,000 bp, at least about 25,000-30,000 bp, at least about 30,000-35,000 bp, at least about 35,000-40,000 bp, at least about 40,000-45,000 bp, at least about 45,000-50,000 bp, at least about 50,000-55,000 bp, at least about 55,000-60,000 bp, at least about 60,000-65,000 bp, at least about 65,000-70,000 bp, at least about 70,000-75,000 bp, at least about 75,000-80,000 bp, at least about 80,000-85,000 bp, at least about 85,000-90,000 bp, at least about 90,000-95,000 bp, at least about 95,000-100,000 bp, at least about 100,000-110,000 bp, at least about 110,000-120,000 bp, at least about 120,000-130,000 bp, at least about 130,000-140,000 bp, at least about 140,000-150,000 bp, at least about 150,000-200,000 bp, or more than at least about 200,000 bp. In one specific embodiment, 9687 bp are inverted. In some embodiments, the inverted nucleotides are interspersed. In some embodiments, the inverted nucleotides are consecutive.

In some embodiments, the one or more inversions are located within at least about 1-500 bp of the phage genome. In some embodiments, the one or more inversions are located within at least about 500-1000 bp of the phage genome. In some embodiments, the one or more inversions are located within at least about 1000-2000 bp of the phage genome. In some embodiments, the one or more inversions are located within at least about 1000-2000 bp of the phage genome. In some embodiments, the one or more inversions are located within at least about 2000-3000 bp of the phage genome. In some embodiments, the one or more inversions are located within at least about 3000-4000 bp of the phage genome. In some embodiments, the one or more inversions are located within at least about 4000-5000 bp of the phage genome. In some embodiments, the one or more inversions are located within at least about 5,000-6,000 bp of the phage genome. In some embodiments, the one or more inversions are located within at least about 6,000-7,000 bp of the phage genome. In some embodiments, the one or more inversions are located within at least about 7,000-8,000 bp of the phage genome. In some embodiments, the one or more inversions are located within at least about 8,000-9,000 bp of the phage genome. In some embodiments, the one or more inversions are located within at least about 9,000-10,000 bp of the phage genome. In some embodiments, the one or more inversions are located within at least about 10,000-15,000 bp of the phage genome. In some embodiments, the one or more inversions are located within at least about 10,000-15,000 bp of the phage genome, at least about 15,000-20,000 bp of the phage genome, at least about 20,000-25,000 bp of the phage genome, at least about 25,000-30,000 bp of the phage genome, at least about 30,000-35,000 bp of the phage genome, at least about 35,000-40,000 bp of the phage genome, at least about 40,000-45,000 bp of the phage genome, at least about 45,000-50,000 bp of the phage genome, at least about 50,000-55,000 bp of the phage genome, at least about 55,000-60,000 bp of the phage genome, at least about 60,000-65,000 bp of the phage genome, at least about 65,000-70,000 bp of the phage genome, at least about 70,000-75,000 bp of the phage genome, at least about 75,000-80,000 bp of the phage genome, at least about 80,000-85,000 bp of the phage genome, at least about 85,000-90,000 bp of the phage genome, at least about 90,000-95,000 bp of the phage genome, at least about 95,000-100,000 bp of the phage genome, at least about 100,000-110,000 bp of the phage genome, at least about 110,000-120,000 bp of the phage genome, at least about 120,000-130,000 bp of the phage genome, at least about 130,000-140,000 bp of the phage genome, at least about 140,000-150,000 bp of the phage genome, at least about 150,000-200,000 bp of the phage genome, or more than at least about 200,000 bp of the phage genome. In one specific embodiment, 9687 bp of the phage genome are inverted. In some embodiments, the inverted nucleotides are interspersed. In some embodiments, the inverted nucleotides are consecutive.

In some embodiments, the inversions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the phage genome. In some embodiments, at least about 30-40% of the phage genome is inverted. In some embodiments, the inversions are located within at least about 40-50% of the phage genome. In some embodiments, the inversions are located within at least about 50-60% of the phage genome. In some embodiments, the inversions are located within at least about 60-70% of the phage genome. In some embodiments, the inversions are located within at least about 70-80% of the phage genome. In some embodiments, the inversions are located within at least about 80-90% of the phage genome. In some embodiments, the inversions are located within at least about 90-100% of the phage genome.

In some embodiments, at least about 1, 2, 3, 4, 5, 6, 7, 8, 9, 10, 11, 12, 13, 14, 15, 16, 17, 18, 19, or 20 genes comprise inversions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise inversions. In some embodiments, 13 genes comprise inversions. In one embodiment, 74 genes comprise inversions.

In some embodiments, the one or more inversions are located at the beginning or 5′ end of the phage genome. In some embodiments, the one or more inversions are located at the end or 3′ end of the phage genome. In some embodiments, the one or more inversions are located in the middle of the phage genome. In some embodiments, the phage genes are interspersed within the bacterial genome and the inversion are located in one or more of the interspersed positions.

In some embodiments, the region for an optimal inversion, i.e., to achieve a desired effect, can be determined through analysis of homology with other phages is other bacteria. Homologous conserved regions in phages may be suitable for inversion, as these are conserved and may comprise one or more essential genes. In some embodiments, regulatory elements, such as promoters, are inverted. In some embodiments, coding sequences are inverted. In some embodiments, the one or more inverted regions contain one or more genes essential for the lytic cycle.

In some embodiments, the inversions are located within one or more genes encoding lytic genes. In some embodiments, the inversions are located within one or more genes encoding one or more proteases or lysins. In some embodiments, the inversions are located within one or more genes encoding one or more toxins. In some embodiments, the inversions are located within one or more genes encoding one or more antibiotic resistance related proteins. In some embodiments, the inversions are located within one or more genes encoding one or phage translation related proteins. In some embodiments, the one or more inversions are located within one or more genes encoding structural proteins. Such structural genes include genes encoding polypeptides of the head, tail, collar, or coat. In some embodiments, the inversions are located within one or more genes encoding one or more plate proteins. In some embodiments, the inversions are located within one or more genes encoding one or more proteins require for assembly of the bacteriophage. In some embodiments, the inversions are located within one or more genes encoding one or more portal proteins. In some embodiments, the inversions are located within one or more genes encoding one or more polypeptides involved in recombination. In some embodiments, the inversions are located within one or more genes encoding one or more integrases. In some embodiments, the inversions are located within one or more genes encoding one or more invertases. In some embodiments, the inversions are located within one or more genes encoding one or more transposases. In some embodiments, the inversions are located with within one or more genes encoding one or more polypeptides involved in replication or translation. In some embodiments, the inversions are located within one or more genes encoding one or more primases. In some embodiments, the inversions are located within one or more genes encoding one or more tRNA related proteins. In some embodiments, the inversions are located within one or more genes encoding one or more polypeptides involved in phage inversion. In some embodiments, the inversions are located within one or more genes encoding an attachment site. In some embodiments, the inversions are located within one or more genes encoding one or more polypeptides involved in packaging. In some embodiments, the inversions are located within one or more genes encoding one or more terminases. In some embodiments, the inversions are located within one or more genes encoding one or more host genes.

In some embodiments, the inversions are located within genes encoding one or more polypeptides involved in one or more of cell lysis, phage structure, phage assembly, phage packaging recombination, replication or translation, phage inversion, or are host proteins, and combinations thereof.

In some embodiments, the inversions are located within genes encoding one or more polypeptides involved in one or more of cell lysis, phage structure, phage assembly, phage packaging recombination, replication or translation, phage inversion, and combinations thereof.

In some embodiments, the inversions are located within 1 genes encoding polypeptides involved in cell lysis, phage structure, phage assembly, phage packaging recombination, replication or translation, phage inversion, and combinations thereof. In some embodiments, the inversions are located within 2 genes encoding polypeptides involved in cell lysis, phage structure, phage assembly, phage packaging recombination, replication or translation, phage inversion, and combinations thereof. In some embodiments, the inversions are located within 3 genes encoding polypeptides involved in cell lysis, phage structure, phage assembly, phage packaging recombination, replication or translation, phage inversion, and combinations thereof. In some embodiments, the inversions are located within 4 genes encoding polypeptides involved in cell lysis, phage structure, phage assembly, phage packaging recombination, replication or translation, phage inversion, and combinations thereof. In some embodiments, the inversions are located within 2 genes encoding polypeptides involved in cell lysis, phage structure, phage assembly, phage packaging recombination, replication or translation, phage inversion, and combinations thereof. In some embodiments, the inversions are located within 5 genes encoding polypeptides involved in cell lysis, phage structure, phage assembly, phage packaging recombination, replication or translation, phage inversion, and combinations thereof. In some embodiments, the inversions are located within 6 genes encoding polypeptides involved in cell lysis, phage structure, phage assembly, phage packaging recombination, replication or translation, phage inversion, and combinations thereof. In some embodiments, the inversions are located within 7 genes encoding polypeptides involved in cell lysis, phage structure, phage assembly, phage packaging recombination, replication or translation, phage inversion, and combinations thereof. In some embodiments, the inversions are located within 8 genes encoding polypeptides involved in cell lysis, phage structure, phage assembly, phage packaging recombination, replication or translation, phage inversion, and combinations thereof. In some embodiments, the inversions are located within 9 genes encoding polypeptides involved in cell lysis, phage structure, phage assembly, phage packaging recombination, replication or translation, phage inversion, and combinations thereof. In some embodiments, the inversions are located within 10 genes encoding polypeptides involved in cell lysis, phage structure, phage assembly, phage packaging recombination, replication or translation, phage inversion, and combinations thereof. In some embodiments, the inversions are located within 11 genes encoding polypeptides involved in cell lysis, phage structure, phage assembly, phage packaging recombination, replication or translation, phage inversion, and combinations thereof. In some embodiments, the inversions are located within 12 genes encoding polypeptides involved in cell lysis, phage structure, phage assembly, phage packaging recombination, replication or translation, phage inversion, and combinations thereof. In some embodiments, the inversions are located within 13 genes encoding polypeptides involved in cell lysis, phage structure, phage assembly, phage packaging recombination, replication or translation, phage inversion, and combinations thereof. In some embodiments, the inversions are located within 14 genes encoding polypeptides involved in cell lysis, phage structure, phage assembly, phage packaging recombination, replication or translation, phage inversion, and combinations thereof. In some embodiments, the inversions are located within 15 genes encoding polypeptides involved in cell lysis, phage structure, phage assembly, phage packaging recombination, replication or translation, phage inversion, and combinations thereof. In some embodiments, the inversions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage inversion, and combinations thereof. In some embodiments, the inversions are located within one or more host proteins within the phage genome.

### Substitutions

In some embodiments, the substitution is in a coding region of the phage genome. In some embodiments, the substitution is substituted into a regulatory region of the phage genome. In some embodiments, the substitutions comprise one or more antibiotic cassette(s). suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more substitutions comprise at least about 1-500 bp. In some embodiments, the one or more substitutions comprise at least about 500-1000 bp. In some embodiments, the one or more substitutions comprise at least about 1000-2000 bp. In some embodiments, the one or more substitutions comprise at least about 1000-2000 bp. In some embodiments, the one or more substitutions comprise at least about 2000-3000 bp. In some embodiments, the one or more substitutions comprise at least about 3000-4000 bp. In some embodiments, the one or more substitutions comprise at least about 4000-5000 bp. In some embodiments, the one or more substitutions comprise at least about 5,000-6,000 bp. In some embodiments, the one or more substitutions comprise at least about 6,000-7,000 bp. In some embodiments, the one or more substitutions comprise at least about 7,000-8,000 bp. In some embodiments, the one or more substitutions comprise at least about 8,000-9,000 bp. In some embodiments, the one or more substitutions comprise at least about 9,000-10,000 bp. In some embodiments, the one or more substitutions comprise at least about 10,000-15,000 bp. In some embodiments, the one or more substitutions comprise at least about 10,000-15,000 bp, at least about 15,000-20,000 bp, at least about 20,000-25,000 bp, at least about 25,000-30,000 bp, at least about 30,000-35,000 bp, at least about 35,000-40,000 bp, at least about 40,000-45,000 bp, at least about 45,000-50,000 bp, at least about 50,000-55,000 bp, at least about 55,000-60,000 bp, at least about 60,000-65,000 bp, at least about 65,000-70,000 bp, at least about 70,000-75,000 bp, at least about 75,000-80,000 bp, at least about 80,000-85,000 bp, at least about 85,000-90,000 bp, at least about 90,000-95,000 bp, at least about 95,000-100,000 bp, at least about 100,000-110,000 bp, at least about 110,000-120,000 bp, at least about 120,000-130,000 bp, at least about 130,000-140,000 bp, at least about 140,000-150,000 bp, at least about 150,000-200,000 bp, or more than at least about 200,000 bp. In one specific embodiment, 9687 bp are substituted. In some embodiments, the substituted nucleotides are interspersed. In some embodiments, the substituted nucleotides are consecutive.

In some embodiments, the one or more substitutions are located within 1-500 bp of the phage genome. In some embodiments, the one or more substitutions are located within at least about 500-1000 bp of the phage genome. In some embodiments, the one or more substitutions are located within at least about 1000-2000 bp of the phage genome. In some embodiments, the one or more substitutions are located within at least about 1000-2000 bp of the phage genome. In some embodiments, the one or more substitutions are located within at least about 2000-3000 bp of the phage genome. In some embodiments, the one or more substitutions are located within at least about 3000-4000 bp of the phage genome. In some embodiments, the one or more substitutions are located within at least about 4000-5000 bp of the phage genome. In some embodiments, the one or more substitutions are located within at least about 5,000-6,000 bp of the phage genome. In some embodiments, the one or more substitutions are located within at least about 6,000-7,000 bp of the phage genome. In some embodiments, the one or more substitutions are located within at least about 7,000-8,000 bp of the phage genome. In some embodiments, the one or more substitutions are located within at least about 8,000-9,000 bp of the phage genome. In some embodiments, the one or more substitutions are located within at least about 9,000-10,000 bp of the phage genome. In some embodiments, the one or more substitutions are located within at least about 10,000-15,000 bp of the phage genome. In some embodiments, the one or more substitutions are located within at least about 10,000-15,000 bp of the phage genome, at least about 15,000-20,000 bp of the phage genome, at least about 20,000-25,000 bp of the phage genome, at least about 25,000-30,000 bp of the phage genome, at least about 30,000-35,000 bp of the phage genome, at least about 35,000-40,000 bp of the phage genome, at least about 40,000-45,000 bp of the phage genome, at least about 45,000-50,000 bp of the phage genome, at least about 50,000-55,000 bp of the phage genome, at least about 55,000-60,000 bp of the phage genome, at least about 60,000-65,000 bp of the phage genome, at least about 65,000-70,000 bp of the phage genome, at least about 70,000-75,000 bp of the phage genome, at least about 75,000-80,000 bp of the phage genome, 80,000-85,000 bp of the phage genome, at least about 85,000-90,000 bp of the phage genome, at least about 90,000-95,000 bp of the phage genome, at least about 95,000-100,000 bp of the phage genome, at least about 100,000-110,000 bp of the phage genome, at least about 110,000-120,000 bp of the phage genome, at least about 120,000-130,000 bp of the phage genome, at least about 130,000-140,000 bp of the phage genome, at least about 140,000-150,000 bp of the phage genome, at least about 150,000-200,000 bp of the phage genome, or more than at least about 200,000 bp of the phage genome. In one specific embodiment, 9687 bp of the phage genome are substituted. In some embodiments, the substituted nucleotides are interspersed. In some embodiments, the substituted nucleotides are consecutive.

In some embodiments, the substitutions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the phage genome. In some embodiments, at least about 30-40% of the phage genome is substituted. In some embodiments, the substitutions are located within at least about 40-50% of the phage genome. In some embodiments, the substitutions are located within at least about 50-60% of the phage genome. In some embodiments, the substitutions are located within at least about 60-70% of the phage genome. In some embodiments, the substitutions are located within at least about 70-80% of the phage genome. In some embodiments, the substitutions are located within at least about 80-90% of the phage genome. In some embodiments, the substitutions are located within at least about 90-100% of the phage genome.

In some embodiments, at least about 1, 2, 3, 4, 5, 6, 7, 8, 9, 10, 11, 12, 13, 14, 15, 16, 17, 18, 19, or 20 genes comprise substitutions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise substitutions. In some embodiments, 13 genes comprise substitutions. In one embodiment, 74 genes comprise substitutions.

In some embodiments, the one or more substitutions are located at the beginning or 5′ end of the phage genome. In some embodiments, the one or more substitutions are located at the end or 3′ end of the phage genome. In some embodiments, the one or more substitutions are located in the middle of the phage genome. In some embodiments, the phage genes are interspersed within the bacterial genome and the substitution are located in one or more of the interspersed positions.

In some embodiments, the region for an optimal substitution, i.e., to achieve a desired effect, can be determined through analysis of homology with other phages is other bacteria. Homologous conserved regions in phages may be suitable for substitution, as these are conserved and may comprise one or more essential genes. In some embodiments, regulatory elements, such as promoters, are substituted. In some embodiments, coding sequences are substituted. In some embodiments, the one or more substituted regions contain one or more genes essential for the lytic cycle.

In some embodiments, the substitutions are located within one or more genes encoding lytic genes. In some embodiments, the substitutions are located within one or more genes encoding one or more proteases or lysins. In some embodiments, the substitutions are located within one or more genes encoding one or more toxins. In some embodiments, the substitutions are located within one or more genes encoding one or more antibiotic resistance related proteins. In some embodiments, the substitutions are located within one or more genes encoding one or phage translation related proteins. In some embodiments, the one or more substitutions are located within one or more genes encoding structural proteins. Such structural genes include genes encoding polypeptides of the head, tail, collar, or coat. In some embodiments, the substitutions are located within one or more genes encoding one or more plate proteins. In some embodiments, the substitutions are located within one or more genes encoding one or more proteins require for assembly of the bacteriophage. In some embodiments, the substitutions are located within one or more genes encoding one or more portal proteins. In some embodiments, the substitutions are located within one or more genes encoding one or more polypeptides involved in recombination. In some embodiments, the substitutions are located within one or more genes encoding one or more integrases. In some embodiments, the substitutions are located within one or more genes encoding one or more invertases. In some embodiments, the substitutions are located within one or more genes encoding one or more transposases. In some embodiments, the substitutions are located with within one or more genes encoding one or more polypeptides involved in replication or translation. In some embodiments, the substitutions are located within one or more genes encoding one or more primases. In some embodiments, the substitutions are located within one or more genes encoding one or more tRNA related proteins. In some embodiments, the substitutions are located within one or more genes encoding one or more polypeptides involved in phage substitution. In some embodiments, the substitutions are located within one or more genes encoding an attachment site. In some embodiments, the substitutions are located within one or more genes encoding one or more polypeptides involved in packaging. In some embodiments, the substitutions are located within one or more genes encoding one or more terminases. In some embodiments, the substitutions are located within one or more genes encoding one or more host genes.

In some embodiments, the substitutions are located within genes encoding one or more polypeptides involved in one or more of cell lysis, phage structure, phage assembly, phage packaging recombination, replication or translation, phage substitution, or are host proteins, and combinations thereof.

In some embodiments, the substitutions are located within genes encoding one or more polypeptides involved in one or more of cell lysis, phage structure, phage assembly, phage packaging recombination, replication or translation, phage substitution, and combinations thereof.

In some embodiments, the substitutions are located within 1 genes encoding polypeptides involved in cell lysis, phage structure, phage assembly, phage packaging recombination, replication or translation, phage substitution, and combinations thereof. In some embodiments, the substitutions are located within 2 genes encoding polypeptides involved in cell lysis, phage structure, phage assembly, phage packaging recombination, replication or translation, phage substitution, and combinations thereof. In some embodiments, the substitutions are located within 3 genes encoding polypeptides involved in cell lysis, phage structure, phage assembly, phage packaging recombination, replication or translation, phage substitution, and combinations thereof. In some embodiments, the substitutions are located within 4 genes encoding polypeptides involved in cell lysis, phage structure, phage assembly, phage packaging recombination, replication or translation, phage substitution, and combinations thereof. In some embodiments, the substitutions are located within 2 genes encoding polypeptides involved in cell lysis, phage structure, phage assembly, phage packaging recombination, replication or translation, phage substitution, and combinations thereof. In some embodiments, the substitutions are located within 5 genes encoding polypeptides involved in cell lysis, phage structure, phage assembly, phage packaging recombination, replication or translation, phage substitution, and combinations thereof. In some embodiments, the substitutions are located within 6 genes encoding polypeptides involved in cell lysis, phage structure, phage assembly, phage packaging recombination, replication or translation, phage substitution, and combinations thereof. In some embodiments, the substitutions are located within 7 genes encoding polypeptides involved in cell lysis, phage structure, phage assembly, phage packaging recombination, replication or translation, phage substitution, and combinations thereof. In some embodiments, the substitutions are located within 8 genes encoding polypeptides involved in cell lysis, phage structure, phage assembly, phage packaging recombination, replication or translation, phage substitution, and combinations thereof. In some embodiments, the substitutions are located within 9 genes encoding polypeptides involved in cell lysis, phage structure, phage assembly, phage packaging recombination, replication or translation, phage substitution, and combinations thereof. In some embodiments, the substitutions are located within 10 genes encoding polypeptides involved in cell lysis, phage structure, phage assembly, phage packaging recombination, replication or translation, phage substitution, and combinations thereof. In some embodiments, the substitutions are located within 11 genes encoding polypeptides involved in cell lysis, phage structure, phage assembly, phage packaging recombination, replication or translation, phage substitution, and combinations thereof. In some embodiments, the substitutions are located within 12 genes encoding polypeptides involved in cell lysis, phage structure, phage assembly, phage packaging recombination, replication or translation, phage substitution, and combinations thereof. In some embodiments, the substitutions are located within 13 genes encoding polypeptides involved in cell lysis, phage structure, phage assembly, phage packaging recombination, replication or translation, phage substitution, and combinations thereof. In some embodiments, the substitutions are located within 14 genes encoding polypeptides involved in cell lysis, phage structure, phage assembly, phage packaging recombination, replication or translation, phage substitution, and combinations thereof. In some embodiments, the substitutions are located within 15 genes encoding polypeptides involved in cell lysis, phage structure, phage assembly, phage packaging recombination, replication or translation, phage substitution, and combinations thereof. In some embodiments, the substitutions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage substitution, and combinations thereof. In some embodiments, the substitutions are located within one or more host proteins within the phage genome.

Phage in E. coli Nissle

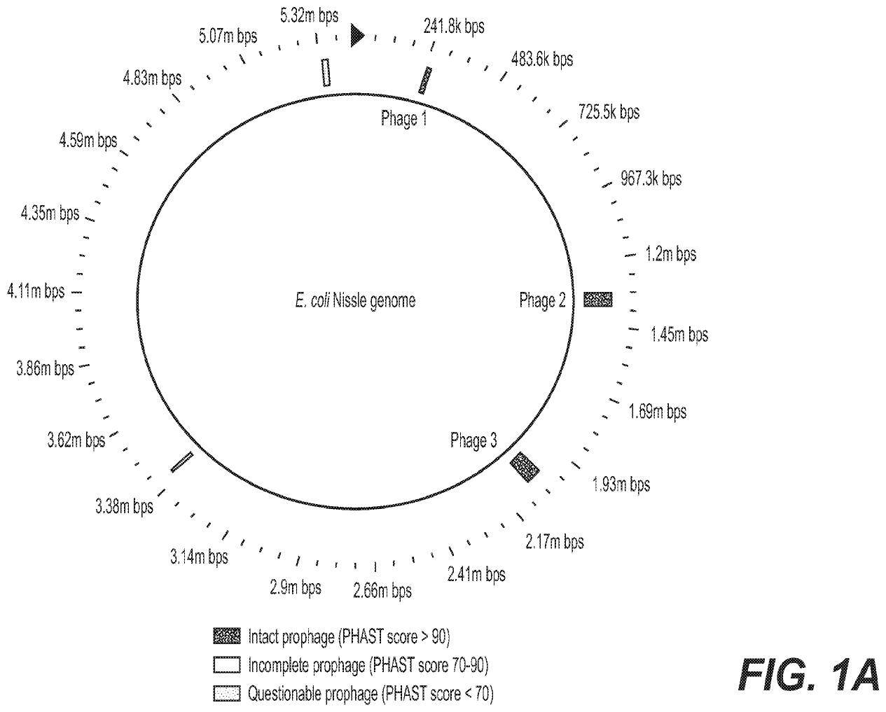

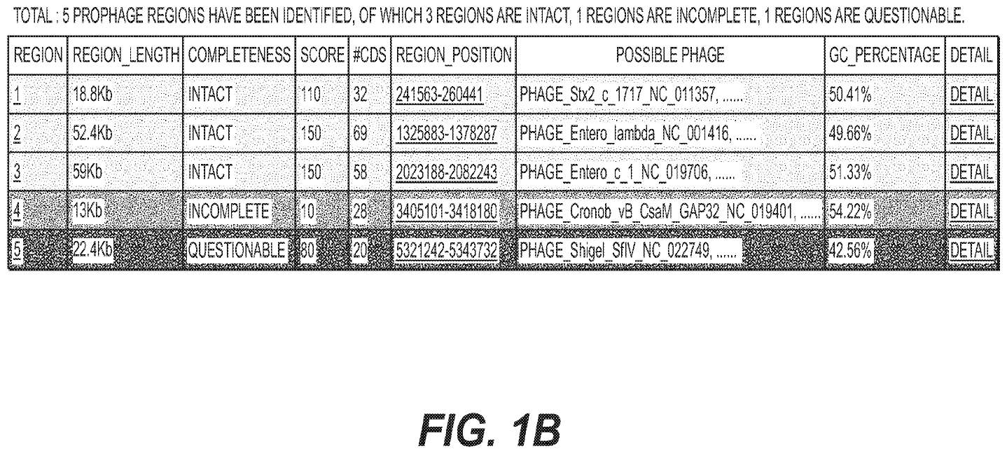

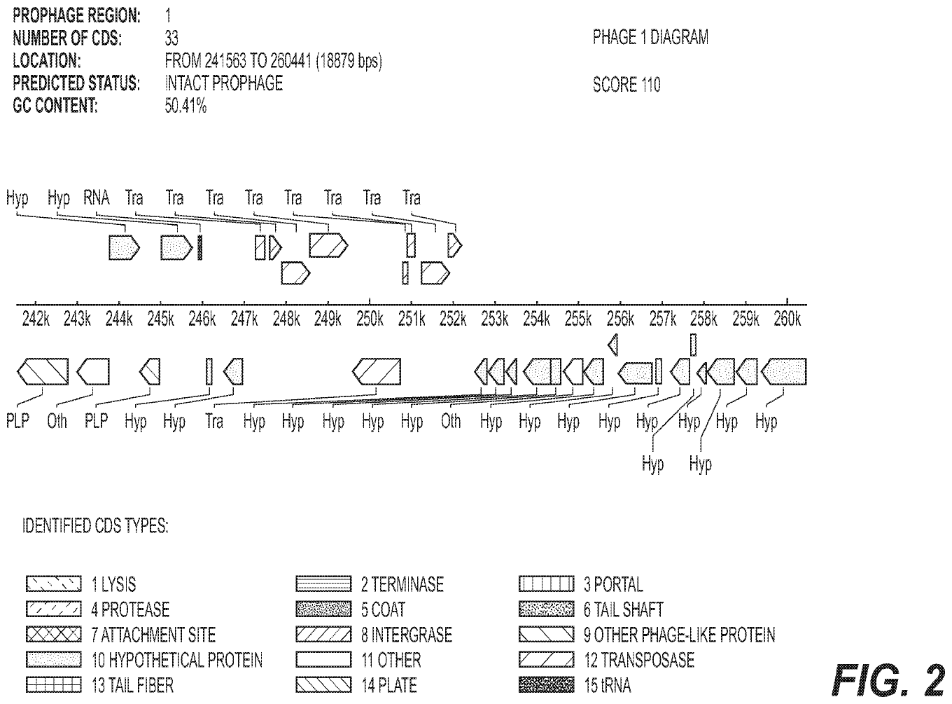

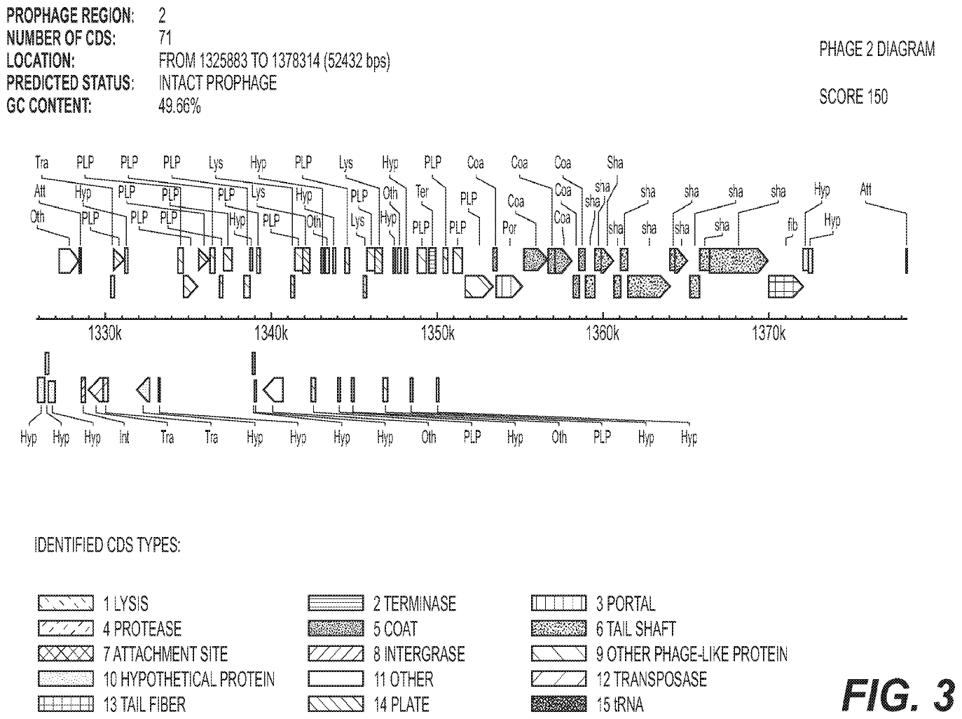

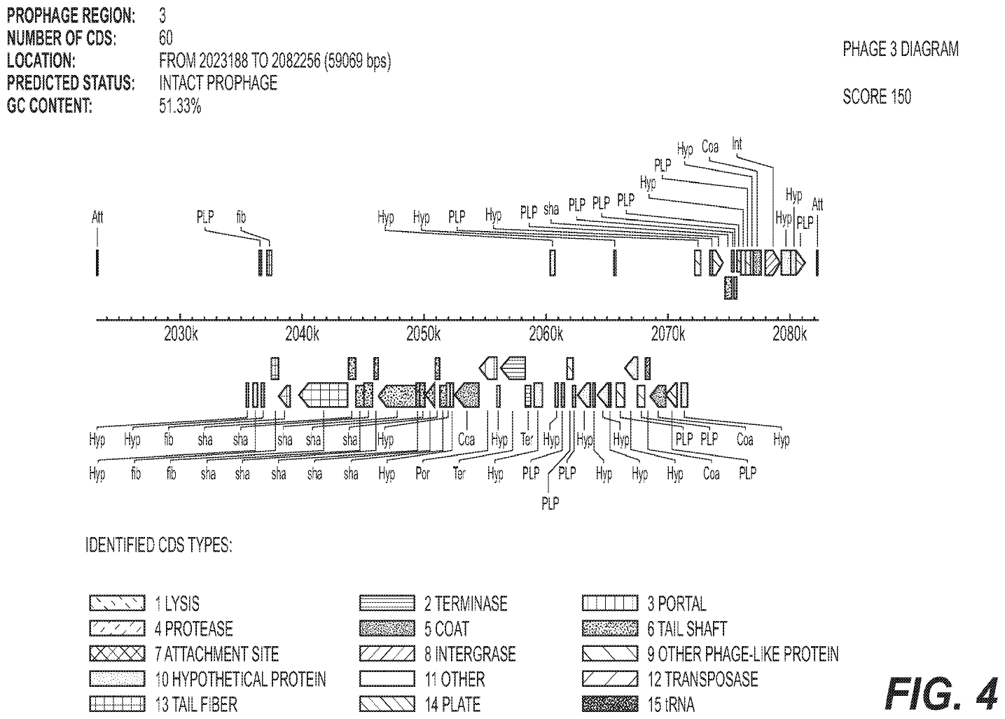

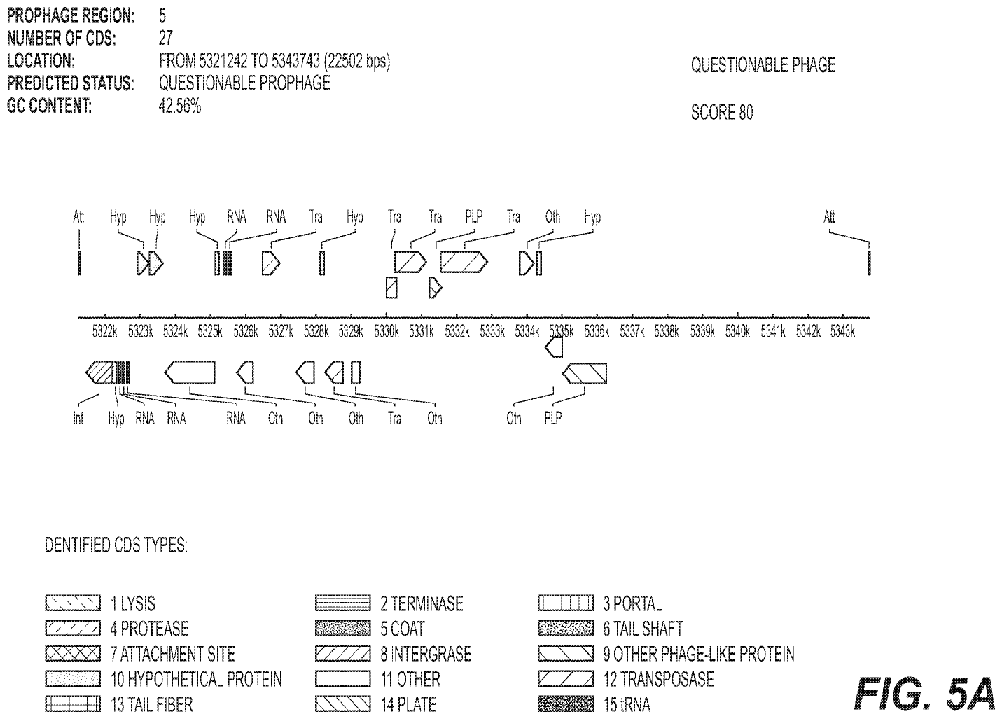

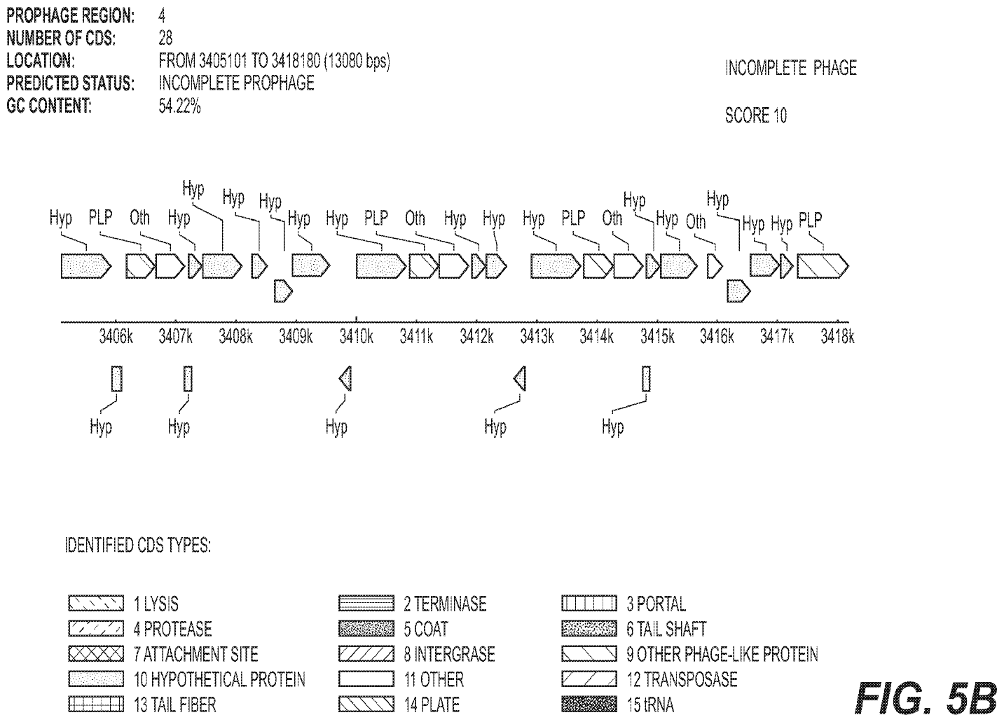

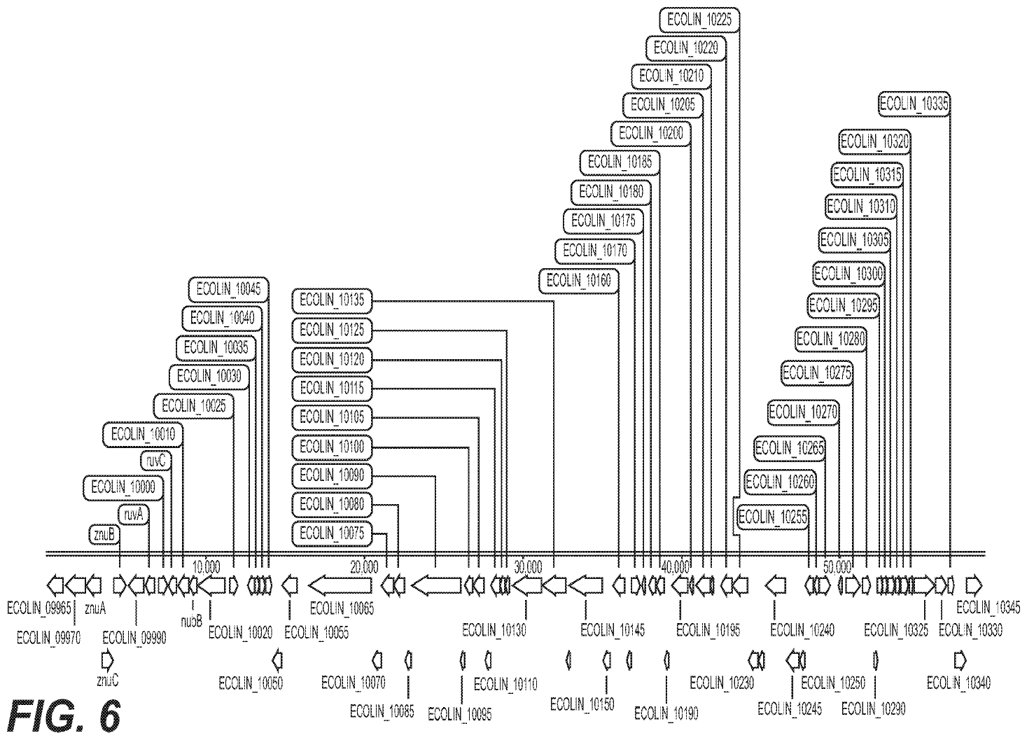

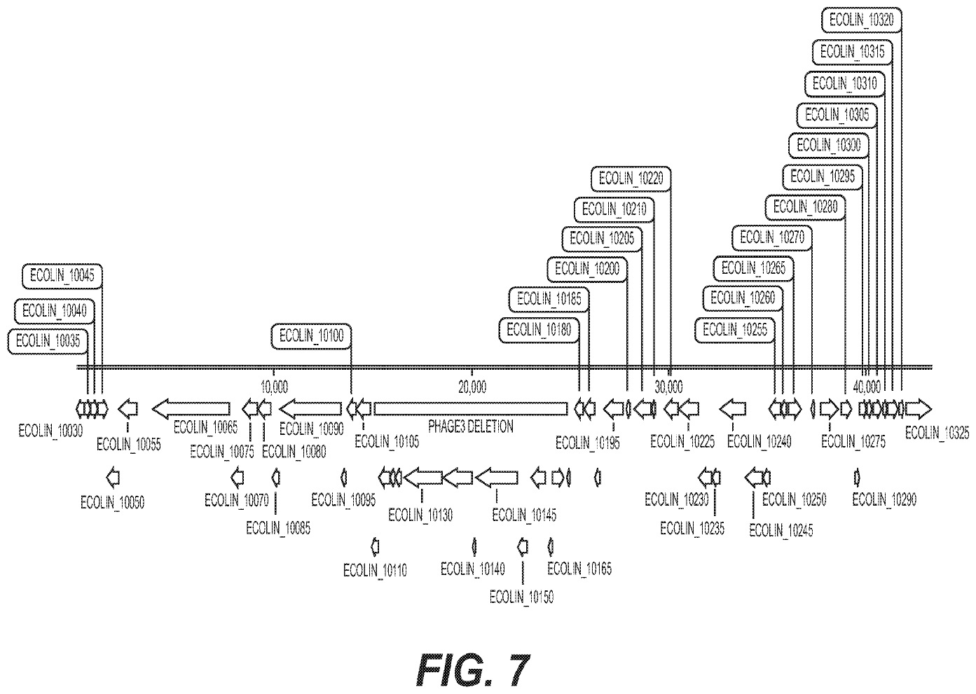

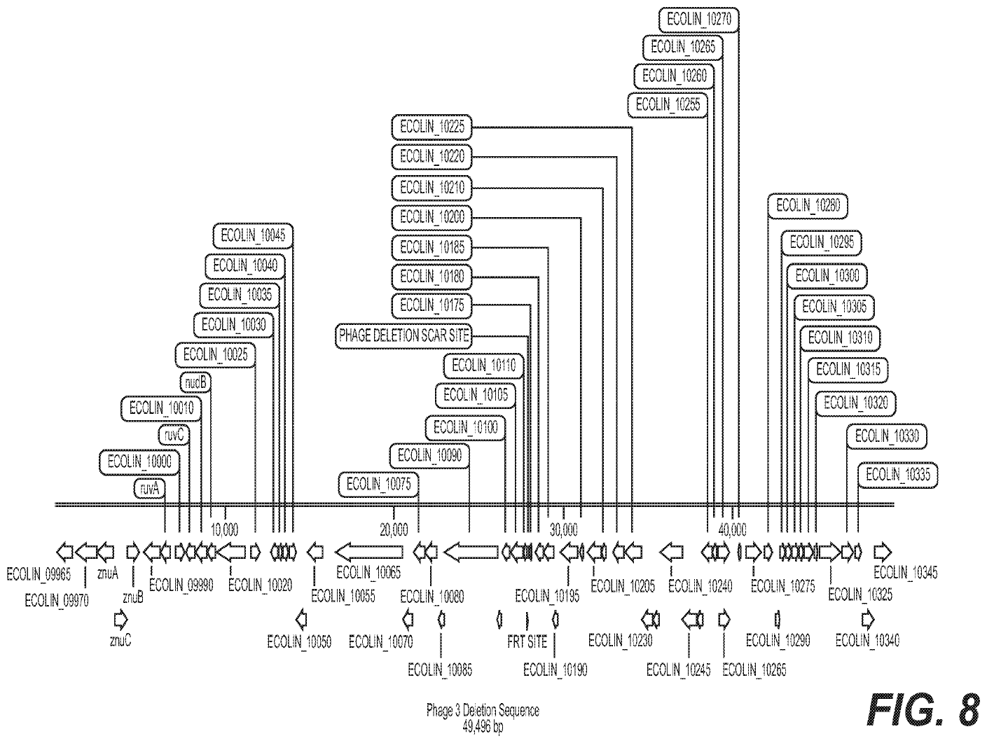

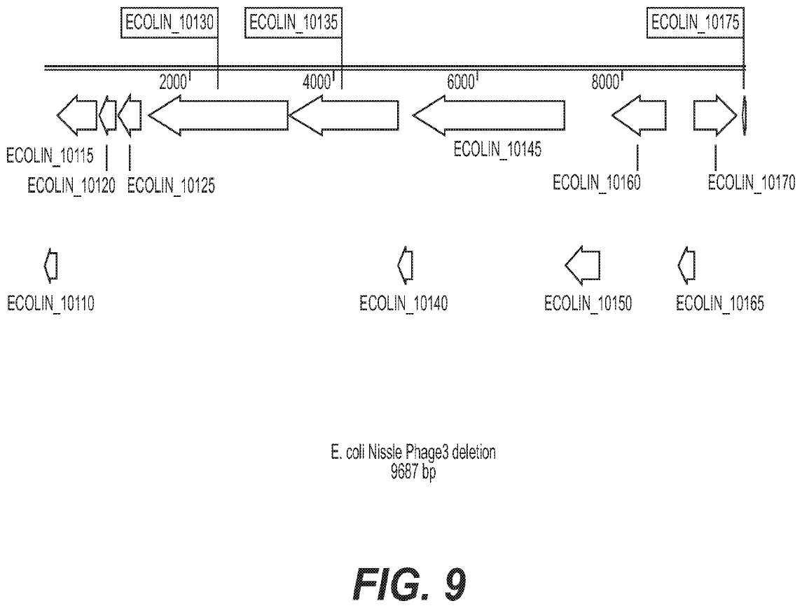

In some embodiments, described herein genetically engineered bacteria are engineered Escherichia coli strain Nissle 1917 (E. coli Nissle). As described in more detail herein in the examples, routine testing procedures identified bacteriophage production from Escherichia coli Nissle 1917 (E. coli Nissle; E. coli Nissle) and related engineered derivatives. To determine the source of the bacteriophage, a collaborative bioinformatics assessment of the genomes of E. coli Nissle, and engineered derivatives was conducted to analyze genomic sequences of the strains for evidence of prophages, to assess any identified prophage elements for the likelihood of producing functional phage, to compare any functional phage elements with other known phage identified among bacterial genomic sequences, and to evaluate the frequency with which prophage elements are found in other sequenced Escherichia coli (E. coli) genomes. The assessment tools included phage prediction software (PHAST and PHASTER), SPAdes genome assembler software, software for mapping low-divergent sequences against a large reference genome (BWA MEM), genome sequence alignment software (MUMmer), and the National Center for Biotechnology Information (NCBI) nonredundant database. The assessment results show that E. coli Nissle and engineered derivatives analyzed contain three candidate prophage elements (FIG. 1), with two of the three (Phage 2 and Phage 3) containing most genetic features characteristic of intact phage genomes (FIG. 2, FIG. 3, and FIG. 4). Two other possible phage elements (FIG. 5A and FIG. 5B) were also identified. Of note, the engineered strains did not contain any additional phage elements that were not identified in parental E. coli Nissle, indicating that plaque-forming units produced by these strains originate from one of these endogenous phages. Further analysis described herein identified Phage 3 as the plaque-forming phage (Phage 3). Interestingly, Phage 3 is unique to E. coli Nissle among a collection of almost 6000 sequenced E. coli genomes, although related sequences limited to short regions of homology with other putative prophage elements are found in a small number of genomes. As described in more detail in the Examples, Phage 3, but not any of the other Phage was found to be inducible and result in bacterial lysis upon induction.

Prophages are very common among E. coli strains, with E. coli Nissle containing a relatively small number of prophage sequences compared to the average number found in a well-characterized set of sequenced E. coli genomes. As such, prophage presence in the engineered strains is part of the natural state of this species and the prophage features of the engineered strains analyzed were consistent with the progenitor strain, E. coli Nissle.

Table D lists the genes contained within the genome of Phage 3. Table E. Provides the sequence of Phage 3. Table F provides the sequences of the genes comprised in Phage 3 of E. coli Nissle. Table G. provides the sequences of the polypeptides encoded by the genome of E. coli Nissle Phage 3.

TABLE D
Phage 3 Genome

| Description | Position | Length | Orientation | GI Number | Protein ID | product |
|---|---|---|---|---|---|---|
| ECOLIN_09965 | 27 . . . | 972 | <= | 660511998 | AID78889.1 | lipid A |
| | 998 | | | | | biosynthesis |
| | | | | | | (KDO)2- |
| | | | | | | (lauroyl)-lipid |
| | | | | | | IVA |
| | | | | | | acyltransferase |
| ECOLIN_09970 | 1117 . . . | 1323 | <= | 660511999 | AID78890.1 | peptidase |
| | 2439 | | | | | |
| ECOLIN_09975 | 2455 . . . | 933 | <= | 660512000 | AID78891.1 | zinc ABC |
| | 3387 | | | | | transporter |
| | | | | | | substrate- |
| | | | | | | binding protein |
| ECOLIN_09980 | 3466 . . . | 756 | => | 660512001 | AID78892.1 | zinc ABC |
| | 4221 | | | | | transporter |
| | | | | | | ATPase |
| ECOLIN_09985 | 4218 . . . | 786 | => | 660512002 | AID78893.1 | high-affinity |
| | 5003 | | | | | zinc transporter |
| | | | | | | membrane |
| | | | | | | component |
| ECOLIN_09990 | 5150 . . . | 1011 | <= | 660512003 | AID78894.1 | ATP-dependent |
| | 6160 | | | | | DNA helicase |
| | | | | | | RuvB |
| ECOLIN_09995 | 6169 . . . | 612 | <= | 660512004 | AID78895.1 | ATP-dependent |
| | 6780 | | | | | DNA helicase |
| | | | | | | RuvA |
| ECOLIN_10000 | 7056 . . . | 603 | => | 660512005 | AID78896.1 | hypothetical |
| | 7658 | | | | | protein |
| ECOLIN_10005 | 7660 . . . | 522 | <= | 660512006 | AID78897.1 | Holliday |
| | 8181 | | | | | junction |
| | | | | | | resolvase |
| ECOLIN_10010 | 8216 . . . | 741 | <= | 660512007 | AID78898.1 | hypothetical |
| | 8956 | | | | | protein |
| ECOLIN_10015 | 8985 . . . | 444 | <= | 660512008 | AID78899.1 | dihydroneopterin |
| | 9428 | | | | | triphosphate |
| | | | | | | pyrophosphatase |
| ECOLIN_10020 | 9430 . . . | 1773 | <= | 660512009 | AID78900.1 | aspartyl-tRNA |
| | 11,202 | | | | | synthetase |
| ECOLIN_10025 | 11,512 . . . | 567 | => | 660512010 | AID78901.1 | hydrolase |
| | 12,078 | | | | | |
| ECOLIN_10030 | 12,680 . . . | 390 | <= | 660512011 | AID78902.1 | DNA |
| | 13,069 | | | | | polymerase V |
| | | | | | | ECOLIN_10030 |
| ECOLIN_10035 | 13,148 . . . | 243 | => | 660512012 | AID78903.1 | MsgA |
| | 13,390 | | | | | |
| ECOLIN_10040 | 13,426 . . . | 381 | => | 660512013 | AID78904.1 | hypothetical |
| | 13,806 | | | | | protein |
| ECOLIN_10045 | 13,808 . . . | 444 | => | 660512014 | AID78905.1 | hypothetical |
| | 14,251 | | | | | protein |
| ECOLIN_10050 | 14,223 . . . | 594 | <= | 660512015 | AID78906.1 | phage tail |
| | 14,816 | | | | | protein |
| ECOLIN_10055 | 14,816 . . . | 933 | <= | 660512016 | AID78907.1 | tail protein |
| | 15,748 | | | | | |
| ECOLIN_10065 | 16,519 . . . | 3927 | <= | 660512017 | AID78908.1 | host specificity |
| | 20,445 | | | | | protein |
| ECOLIN_10070 | 20,488 . . . | 618 | <= | 660512018 | AID78909.1 | tail protein |
| | 21,105 | | | | | |
| ECOLIN_10075 | 21,098 . . . | 720 | <= | 660512019 | AID78910.1 | peptidase P60 |
| | 21,817 | | | | | |
| ECOLIN_10080 | 21,820 . . . | 738 | <= | 660512020 | AID78911.1 | hypothetical |
| | 22,557 | | | | | protein |
| ECOLIN_10085 | 22,614 . . . | 339 | <= | 660512021 | AID78912.1 | tail protein |
| | 22,952 | | | | | |
| ECOLIN_10090 | 22,949 . . . | 3138 | <= | 660512022 | AID78913.1 | tail protein |
| | 26,086 | | | | | |
| ECOLIN_10095 | 26,070 . . . | 273 | <= | 660512023 | AID78914.1 | tail protein |
| | 26,342 | | | | | |
| ECOLIN_10100 | 26,393 . . . | 432 | <= | 660512024 | AID78915.1 | tail protein |
| | 26,824 | | | | | |
| ECOLIN_10105 | 26,835 . . . | 744 | <= | 660512025 | AID78916.1 | tail fiber |
| | 27,578 | | | | | protein |
| ECOLIN_10110 | 27,588 . . . | 402 | <= | 660512026 | AID78917.1 | Minor tail |
| | 27,989 | | | | | protein U |
| ECOLIN_10115 | 27,986 . . . | 573 | <= | 660512027 | AID78918.1 | tail protein |
| | 28,558 | | | | | |
| ECOLIN_10120 | 28,574 . . . | 243 | <= | 660512028 | AID78919.1 | DNA breaking- |
| | 28,816 | | | | | rejoining |
| | | | | | | protein |
| ECOLIN_10125 | 28,842 . . . | 327 | <= | 660512029 | AID78920.1 | hypothetical |
| | 29,168 | | | | | protein |
| ECOLIN_10130 | 29,251 . . . | 1947 | <= | 660512030 | AID78921.1 | peptidase S14 |
| | 31,197 | | | | | |
| ECOLIN_10135 | 31,211 . . . | 1500 | <= | 660512031 | AID78922.1 | capsid protein |
| | 32,710 | | | | | |
| ECOLIN_10140 | 32,707 . . . | 216 | <= | 660512032 | AID78923.1 | hypothetical |
| | 32,922 | | | | | protein |
| ECOLIN_10145 | 32,919 . . . | 2103 | <= | 660512033 | AID78924.1 | DNA |
| | 35,021 | | | | | packaging |
| | | | | | | protein |
| ECOLIN_10150 | 35,021 . . . | 489 | <= | 660512034 | AID78925.1 | terminase |
| | 35,509 | | | | | |
| ECOLIN_10160 | 35,693 . . . | 729 | <= | 660512035 | AID78926.1 | hypothetical |
| | 36,421 | | | | | protein |
| ECOLIN_10165 | 36,596 . . . | 231 | <= | 660512036 | AID78927.1 | hypothetical |
| | 36,826 | | | | | protein |
| ECOLIN_10170 | 36,825 . . . | 597 | => | 660512037 | AID78928.1 | hypothetical |
| | 37,421 | | | | | protein |
| ECOLIN_10175 | 37,490 . . . | 198 | <= | 660512038 | AID78929.1 | hypothetical |
| | 37,687 | | | | | protein |
| ECOLIN_10180 | 37,901 . . . | 480 | <= | 660512039 | AID78930.1 | hypothetical |
| | 38,380 | | | | | protein |
| ECOLIN_10185 | 38,401 . . . | 549 | <= | 660512040 | AID78931.1 | lysozyme |
| | 38,949 | | | | | |
| ECOLIN_10190 | 38,921 . . . | 279 | <= | 660512041 | AID78932.1 | holin |
| | 39,199 | | | | | |
| ECOLIN_10195 | 39,345 . . . | 1053 | <= | 660512042 | AID78933.1 | DNA adenine |
| | 40,397 | | | | | methylase |
| ECOLIN_10200 | 40,548 . . . | 192 | <= | 660512043 | AID78934.1 | hypothetical |
| | 40,739 | | | | | protein |
| ECOLIN_10205 | 40,908 . . . | 900 | <= | 660512044 | AID78935.1 | serine protease |
| | 41,807 | | | | | |
| ECOLIN_10210 | 41,820 . . . | 207 | <= | 660512045 | AID78936.1 | hypothetical |
| | 42,026 | | | | | protein |
| ECOLIN_10220 | 42,459 . . . | 690 | <= | 660512046 | AID78937.1 | antitermination |
| | 43,148 | | | | | protein |
| ECOLIN_10225 | 43,170 . . . | 996 | <= | 660512047 | AID78938.1 | hypothetical |
| | 44,165 | | | | | protein |
| ECOLIN_10230 | 44,162 . . . | 684 | <= | 660512048 | AID78939.1 | antirepressor |
| | 44,845 | | | | | |
| ECOLIN_10235 | 44,859 . . . | 387 | <= | 660512049 | AID78940.1 | crossover |
| | 45,245 | | | | | junction |
| | | | | | | endodeoxyribonuclease |
| ECOLIN_10240 | 45,242 . . . | 1320 | <= | 660512050 | AID78941.1 | adenine |
| | 46,561 | | | | | methyltransferase, |
| | | | | | | DNA |
| | | | | | | methyltransferase |
| | | | | | | ECOLIN_10240 |
| ECOLIN_10245 | 46,558 . . . | 882 | <= | 660512051 | AID78942.1 | GntR family |
| | 47,439 | | | | | transcriptional |
| | | | | | | regulator |
| | | | | | | ECOLIN_10245 |
| ECOLIN_10250 | 47,449 . . . | 339 | <= | 660512052 | AID78943.1 | hypothetical |
| | 47,787 | | | | | protein |
| ECOLIN_10255 | 47,784 . . . | 564 | <= | 660512053 | AID78944.1 | hypothetical |
| | 48,347 | | | | | protein, |
| | | | | | | completely |
| | | | | | | unknown |
| ECOLIN_10260 | 48,379 . . . | 258 | <= | 660512054 | AID78945.1 | hypothetical |
| | 48,636 | | | | | protein, cI |
| | | | | | | repressor |
| | | | | | | ECOLIN_10260 |
| ECOLIN_10265 | 48,715 . . . | 711 | => | 660512055 | AID78946.1 | hypothetical |
| | 49,425 | | | | | protein, |
| | | | | | | Domain of |
| | | | | | | unknown |
| | | | | | | function |
| | | | | | | (DUF4222); |
| | | | | | | This short |
| | | | | | | protein is likely |
| | | | | | | to be of phage |
| | | | | | | origin. For |
| | | | | | | example it is |
| | | | | | | found in |
| | | | | | | Enterobacteria |
| | | | | | | phage YYZ- |
| | | | | | | 2008. It is |
| | | | | | | largely found |
| | | | | | | in enteric |
| | | | | | | bacteria. The |
| | | | | | | molecular |
| | | | | | | function of this |
| | | | | | | protein is |
| | | | | | | unknown. |
| ECOLIN_10270 | 49,868 . . . | 198 | <= | 660512056 | AID78947.1 | hypothetical |
| | 50,065 | | | | | protein |
| ECOLIN_10275 | 50,378 . . . | 918 | => | 660512057 | AID78948.1 | DNA |
| | 51,295 | | | | | recombinase |
| | | | | | | In Escherichia |
| | | | | | | coli, RdgC is |
| | | | | | | required for |
| | | | | | | growth in |
| | | | | | | recombination- |
| | | | | | | deficient |
| | | | | | | exonuclease- |
| | | | | | | depleted |
| | | | | | | strains. Under |
| | | | | | | these conditions, |
| | | | | | | RdgC may act as |
| | | | | | | an exonuclease to |
| | | | | | | remove collapsed |
| | | | | | | replication |
| | | | | | | forks, in the |
| | | | | | | absence of the |
| | | | | | | normal repair |
| | | | | | | mechanisms |
| | | | | | | ECOLIN_10275 |
| ECOLIN_10280 | 51,404 . . . | 540 | => | 660512058 | AID78949.1 | hypothetical |
| | 51,943 | | | | | protein, 5′ |
| | | | | | | Deoxynucleotidase |
| | | | | | | YfbR and HD |
| | | | | | | superfamily |
| | | | | | | hydrolases |
| | | | | | | ECOLIN_10280 |
| ECOLIN_10290 | 52,104 . . . | 255 | => | 660512059 | AID78950.1 | hypothetical |
| | 52,358 | | | | | protein |
| | | | | | | Multiple |
| | | | | | | Antibiotic |
| | | | | | | Resistance |
| | | | | | | Regulator |
| | | | | | | (MarR) family of |
| | | | | | | transcriptional |
| | | | | | | regulators |
| ECOLIN_10295 | 52,355 . . . | 348 | => | 660512060 | AID78951.1 | hypothetical |
| | 52,702 | | | | | protein, |
| | | | | | | unknown ead like |
| | | | | | | protein in P22 |
| ECOLIN_10300 | 52,704 . . . | 309 | => | 660512061 | AID78952.1 | hypothetical |
| | 53,012 | | | | | protein, totally |
| | | | | | | unknown |
| ECOLIN_10305 | 53,026 . . . | 468 | => | 660512062 | AID78953.1 | hypothetical |
| | 53,493 | | | | | protein, |
| | | | | | | Protein of |
| | | | | | | unknown |
| | | | | | | function |
| | | | | | | (DUF550); |
| | | | | | | This family is |
| | | | | | | found in a |
| | | | | | | range of |
| | | | | | | Proteobacteria |
| | | | | | | and a few P-22 |
| | | | | | | dsDNA virus |
| | | | | | | particles. The |
| | | | | | | function is |
| | | | | | | currently not |
| | | | | | | known. Similar |
| | | | | | | to P22 EA gene |
| | | | | | | ECOLIN_10305 |
| ECOLIN_10310 | 53,496 . . . | 255 | => | 660512063 | AID78954.1 | hypothetical |
| | 53,750 | | | | | protein, Phage |
| | | | | | | repressor |
| | | | | | | protein C, |
| | | | | | | contains |
| | | | | | | Cro/C1-type |
| | | | | | | HTH and |
| | | | | | | peptisase s24 |
| | | | | | | domains |
| ECOLIN_10315 | 53,772 . . . | 570 | => | 660512064 | AID78955.1 | hypothetical |
| | 54,341 | | | | | protein, 3′-5′ |
| | | | | | | exonuclease |
| | | | | | | ECOLIN_10315 |
| ECOLIN_10320 | 54,382 . . . | 237 | => | 660512065 | AID78956.1 | excisionase |
| | 54,618 | | | | | ECOLIN_10320 |
| ECOLIN_10325 | 54,677 . . . | 1314 | => | 660512066 | AID78957.1 | integrase, |
| | 55,990 | | | | | Phage integrase |
| | | | | | | family; |
| | | | | | | Members of |
| | | | | | | this family |
| | | | | | | cleave DNA |
| | | | | | | substrates by a |
| | | | | | | series of |
| | | | | | | staggered XerC |
| ECOLIN_10330 | 56,017 . . . | 726 | => | 660512067 | AID78958.1 | hypothetical |
| | 56,742 | | | | | protein |
| ECOLIN_10335 | 56,795 . . . | 396 | => | 660512068 | AID78959.1 | membrane |
| | 57,190 | | | | | protein |
| ECOLIN_10340 | 57,231 . . . | 744 | => | 660512069 | AID78960.1 | tRNA |
| | 57,974 | | | | | methyltransferase |
| ECOLIN_10345 | 57,971 . . . | 972 | => | 660512070 | AID78961.1 | tRNA |
| | 58,942 | | | | | methyltransferase |

TABLE E
Phage 3 Genome Sequence
SEQ ID NO: 134

aggcctctcctcgcgagaggcattttttatttgatgggataaagatcttt
gcgcttatacggttggatttcgcccggtttgcgagttttcagcaatttta
atatccaggtgtattgttctggtcgcggaccaacaaaaatctcgacttct
tcattcatccgccgcgcaatcgtatgatcatccgcctctaacagatcatc
catcggtgggcgcacctgaatcgtcagacgatgcgtcttgccatcataaa
tcggaaatagcggtacaacgcgcgcacggcacactttcatcaaacgacca
atcgcgggcaacgtcgctttataggtggcaaagaaatcaacaaattcgct
gtgttctgggccatgatcctgatcgggtaaataatatccccagtaaccct
gacgtaccgactggatgaatggtttaataccatcatttctcgcatgcaga
cgaccaccaaagcgacggcgcaccgtgttccagacataatcaaaaaccgg
gttgccctgattatggaacatcgctgccattttctgcccttgcgaggcca
tcagcatggcaggaatatcgacggcccaaccgtgcggcaccagaaaaatc
actttctcgttattacgtcgtatctcttcgatgatctccagcccttgcca
gtcaacgcgcggctgaattttctccggcccgcgtattgccaactcagcca
tcattaccatcgcttgcggcgcggtggcaaacatctcatctacaatcgct
tcgcgttcagcttcactacgttctggaaagcagagcgacagattgattaa
cgcacgacggcgtgagctttttcccagtcgtccggcaaaacgtcccagcc
gtgccagaatgggatcacggaactttggcggcgttaaagcgatacccgcc
atcgctgctacgcccagccatgctccccagtagcgcgggtggcgaaagga
tttatcaaactcaggaatgtattcgctattattttttttcgtttccatgc
ttttccagtttcggataaggcaaaaatcaatctggtgatagtgtagcggc
gcaacttgccccgcaccaaataaaaaagccggtactgactgcgtaccggc
tgcgaatggatgttaattaatcaaaccgtagctgcggcacaatctctttg
gcctgtgccaggaattcgcgacgatcggagccggtcagcccttcggtacg
cggcagttttgccgtcagcgggtttacggcctgctggtttatccatactt
catagtgcagatgcggcccggttgaacgtccggtattaccggaaagcgcg
atacggtcgccacgtttcaccttctgtcccggtttcaccaggatcttgcg
caagtgcatataacgcgtggtgtagctgcgaccatgacgaatagccacat
aataacctgctgcgccactacgtttggcaaccaccacttcaccgtcaccc
actgaaagcactggcgtaccttgtggcatggcaaaatcaacacctctgtg
tggcgcaacgcgaccggtcaccggattagtacgacgcgggttaaagttag
atgagatacggaactgtttcgccgtcgggaatcgcaagaatcctttcgcc
agaccagtaccgttacgatcgtagaatttgccatcttcagcgcggattgc
gtaataatctttaccttctgaacgcaaacgtacgcccagcagctggcttt
gctcacgtttaccatcaagcatttctcgtgacattaacaccgcaaattca
tcgccttttttcagtttgcggaaatccatttgccactgcatggctttaat
cactgcgctcacttcggcgctggttaaaccggcgtttctggcgctggcaa
caaagcttcccccgacggtacctttcagcagattgttgacccactctcct
tgctgcatttcgctggtcattttaaaaccgttagcggcagtacggtcata
ggttcgggtttcacgacgagacacttcccaggtgaggcgctgcagttcgc
cgtccgcggttaatgtccaggagagttgttgaccgattttcaggttacgc
aattctttgtcggcagcagccagttgggtgatatcacccatatcaatacc
atactgattgagaatgctgcttagcgtatcgccagtggaaacaacatatt
catgcacgcccgcttcaccggcgattttgtcatccagttcgtcctgggga
atggcttcatcttcttgtgcagcttgatcaatcggctcactggcttcagg
taagagcgaacgaatttcgttctgttccagctcaatggttttgacaattg
gcgtggcatcgcggtgataaacatagggccgccagacagcgacggccaga
gtaagaacggtgagcgaccccaacataacgcggtgtggtcgcggtaaatt
attaaacgccagggcgacagagcgggctatctgttgcacgtaatcacttc
ctcattaatctcctttcaggcagctcgcatactggttggctaattgattc
aggaattctgaatagcttgttttacccagtttgatattcgtccccagggg
atccaacgttcccatacgaacggatgtccctcgtgcgacgctctcaacga
ccgctggcctgaactgtggctcagcaaaaacgcaggttgctttttgctca
accaactgtgttcttatttcatgtaaacgctgcgcgccaggttgaatctc
agggttaacggtaaaatgaccaagcggtgtcagtccgaactgtttttcga
aatagccgtaagcatcgtgaaaaacgaaataacctttccccttgagcggc
gcgagctcgttaccaacctgcttttcggttgaggctaattgtgcctcaaa
atccttcaggttggcgtcaagtttggctcgactttgcggcataagttcca
ctaattttccatggattgcaaccgctgtagcccgcgctatctctggggaa
agccaaagatgcatgttgaaatcgccgtgatggtgatcttcgtcactttt
ttccgcgtggtcgtgatcatcatcatcgccgtgaatacttttcatcagca
gcggtttcacattctctagctgcgcaatcgttacctgtttcgcttcaggt
aatttacttaccggtttttgcatgaacgcttccatctccgggccaaccca
aacgactaagtccgcgttctgtaagcgttttacatctgatggacgcagtg
aataatcatgttctgaagccccgtcaggtagtaaaacctccgtttctgtt
accccatcagcaatggcagaagcgatgaacccaacgggtttaagcgaagc
gacaacggcagcatctgcggcctgtgttgcaccgccccagagagcggcgg
ataatgctgcgaaaagaagcgtttttttatgtaacataatgcgaccaatc
atcgtaatgaatatgagaagtgtgatattataacatttcatgactactgc
aagactaaaattaacatgacaagtctggtttccctggaaaatgtctcggt
ttcttttggccaacgccgcgtcctctctgatgtgtcgctggaacttaaac
ctggaaaaattttgactttacttgggccaaacggcgcaggtaagtcgaca
ctggtacgggtagtgctcgggctggtaacacccgatgaaggggttatcaa
gcgcaacggaaaactgcgcatcggctatgtaccgcagaagctgtatctcg
acaccacgttgccactgaccgtaaaccgttttttacgcttacgccctggc
acacataaagaagatattttgcctgcactgaaacgtgtccaggccgggca
tctgattaacgcaccgatgcaaaagctctcgggtggcgaaacgcagcgtg
tactgttagcgcgagcattgttaaatcgaccgcaattattagtgctggat
gaacccactcagggcgtggatgtgaatggtcaggtggcgttatatgacct
tattgaccaactgcgtcgcgaactggattgtggcgttttaatggtatctc
acgatctgcatctggtaatggcaaaaaccgatgaagtgctttgcctgaat
caccacatttgttgttccggcacaccggaagttgtttccctgcatccgga
gtttatttctatgtttggtcctcgtggtgctgaacaactgggtatctatc
gccatcatcataatcatcgtcacgatttacagggacgaattgttttgcgt
cggggaaatgatcgctcatgattgaattattatttcccggttggttagcc
gggatcatgctcgcctgtgccgcgggtccgctgggttcgtttgtagtctg
gcgtcgtatgtcttatttcggtgatacgctggctcatgcctcattacttg
gcgtcgcgtttggtttgttgctggacgtgaatccattctatgcggtgatt
gccgttacgctgctgctggcgggcggtctggtatggctggagaagcgtcc
acagctggcgatcgacacgttattagggattatggcgcacagtgccctgt
cgctgggcctggtggtcgttagtctgatgtctaatattcgtgttgatttg
atggcttacctgttcggtgatttactggcagtgacgccagaagatctcat
ctctattgcgattggcgtggtcatcgtggtggctattttgttctggcaat
ggcgcaatttgctgtcgatgacgattagcccggatctggcgtttgttgat
ggtgtgaaattacagcgcgtgaaattgttgttgatgctggtgacggcatt
gacgattggtgtagcgatgaaattcgtcggcgcgttgattattacttcac
tgctgattattcctgctgctactgcacgtcgctttgcccgcacgccggaa
cagatggctggtgtcgctgttttggtggggatggtggcagtgactggcgg
tttaaccttttccgcattttacgatacacctgcaggcccgtcggtggtgc
tatgcgcggcactgttatttattatcagtatgatgaaaaagcaggccagc
taatctgtcgctgaacacatttgtcggatgcggcgcgagcgccttatccc
acctgcggttcgctatctctggtaggcctgataagacgcgaacagcgtcg
catcaggcacactgccagtgtcggatgcggctcgagcgaccaatccgact
tacggcatttctggcggcgtgatgccgaagtggttccacgcccgcactgt
cgccatacgcccgcgcggtgtacgctgcaaaaagccttgctgaatcaaat
aaggttccagtacatcctcaatggtttcacgttcttcgccaatggctgcc
gccaggttatccagacctaccggcccaccaaagaacttatcgattaccgc
cagcaacaatttgcggtccatataatcgaaaccttcagcatcgacattca
acatatccagcgcctgagcagcgatatctgccgagatggtgccatcgtgc
ttcacttcagcgaaatcacgcactcgacgcagcagacggttggcaatacg
tggcgtaccgcgcgcacgacgagcaacttccagcgcgccgtcatcactca
tctcaagccccataaagcgtgcgctgcgactgacgatatattgcagatcc
ggcacctgataaaactccagacgttgcacaataccaaaacgatcgcgcaa
cggtgatgtcagcgaacctgcgcgcgtggttgcaccaatcagggtaaacg
gcggcaaatcaattttaatggagcgtgccgccggaccttcaccaatcatg
atatccagttggtaatcttccattgccggatacaacacctcttccaccac
tggtgaaagacggtggatctcatcaataaacagtacatcgtgtggttcaa
ggttagtgagcattgctgccagatcgcccgccttttccagcaccggacca
gaagtcgtgcgtaaattaacgcccatttcattggcgacaatattggcaag
cgtagttttacccaaccccggaggaccaaaaatcaatagatgatcgaggg
catcgccgcgcagtttcgctgctttgatgaaaatctccatctgcgaacga
acctgcggctgaccaacatactcttccagtaatttagggcgaatggcgcg
atctgccacatcttccggcaaagtggtaccggcagaaatcagacggtctg
cttcaatcatcctttacctcataacgcggcgcgtagggcttcgcgaatta
atgtttcactgctggcgtcagggcgagcgattttgctcaccatgcggctt
gcttcttgtggtttatagcccagtgccaccagcgcagcaaccgcttcctg
ttcagcatcgtcggtcgccgggctggcaggagacgtgagtaccaggtcgg
cggctggcgtaaagagatcgccatgcaaacctttaaatcggtctttcatt
tcgacaatcaagcgttcggcggtttttttgccaatacccggcagtttcac
cagtgcccccacttcttcacgctcaacggcattaacgaactgctgcgctg
acattccggagaggatcgccagcgccaacttcgggccgacgccgttggtt
ttgatcaactctttgaacaacgtgcgctcttgtttattgttaaaaccgta
cagcagttgcgcgtcttcacgcaccacaaagtgggtgaaaacgatcgctt
cctgacccgcttcagggagttcataaaaacaggtcatcggcatatgcact
tcatagcctacgccgcccacttcaattaacaccagcgggggttgtttttc
aatgatgatgcctctgagtctgcctatcacatgacgctcctgcgtaatga
atcaaagataatgctgtatgataaaaaaatgctggatagatatccagcga
aggatgaagaaaacttgcgaggtgtctcgatgatctgaaaaatggcgcag
tataatttattctacagattatattggaagcaaatatttaaatattacat
attcagcgaagaaatgtgtaataaaaatacacattgcgacccctgaaaaa
aataaattttttatgctattacgtatattcatatctatttcaatggaatg
acaacgtgaatattaattatcctgctgaatatgaaattggtgatatcgtc
tttacatgtataagtgctgccttatttggtcaaatatcagctgcatcaaa
ttgctggagtaatcacgtcgggatcattatcggtcataacggtgaagact
ttctggttgcagaaagccgtgttcccctctcaaccatcactacgctatcc
cgttttattaaacgctctgctaatcaacgctatgctataaagcgattaga
cgccggactaacagaacaacaaaatcaacgaattgttgaacaggttcctt
cccggctacgcaaaatttaccacaccggttttaaatacgaatcttcgcgc
cagttctgttcaaaatttgtttttgatatttataaagaggcgctatgtat
tccggtgggtgaaatagagacgtttggagaattgttaaatagcaatccaa
atgcaaaactcactttctggaaattctggttcttaggttctattccgtgg
gagcgtaaaaccgtcacgccagccagtttgtggcatcatccgggtttggt
gttgattcacgcggtgggagttgaaacgcctcagcctgaactgaccgagg
cggtataacttaacgcagtcgccctctcgccaggttcagtcgcgattcgc
tcatttgcatcgcattctgactaacgtggcagtgggtgatggcaatcgcc
agcgcatcggcggcatccgcctgtggattagcgggcagtttcagcaaggt
gcggaccatatgctgcacctggcttttttcggcactaccaatacctacca
ctgtttgctttacctgacgtgccgcatattcaaataccggcaattcctga
ttcaccgccgccacaatcgccacgccgcgcgcctgccccagtttcagggc
tgagtcagcgttcttcgccataaagacctgttcaatggcgaaataatcag
gctggaattgggtgatgatttccgtcacgcccgcatagatgagcttcaga
cgagacggtaaatcatccactttggtgcgtatgcatccgctacccaggta
ggacagttgcctgcctacctggcggatgacgccatagccggtcacgcgcg
aacccgggtcaatgccgagaataatagccatcacgcgtctccgttttgct
gtttagcaggcctcatcagagagtcgctgcaacctcatcagagatttcac
cgttatggtaaacttcctgcacgtcgtcgcaatcttccagcatatcgatc
agacgcatcagtttcggtgcggtttctgcatccatatcagctttggtgga
cgggatcatggaaacttccgcgctgtctgctttcagacctgccgcttcca
gagcgtcgcgtactttgcccatttcttcccatgcagtgtagacatcaatc
gcgccgtcatcataggtcacaacgtcttcagcaccggcttccagggctgc
ttccatgatggtgtcttcatcgcctttctcgaaggagatcacgccttttt
tgctgaacaaataagctacggaaccatcagtaccgaggttaccgccacat
ttgctgaatgcatgacgcacttcagcaacggtacggttgcggttgtcaga
cagacattcaatcatgattgccgtgccgccaggaccgtaaccttcgtaga
tgatggtttccatgtttgcatcatcatcaccgcccacacctcgtgcaatt
gcgcggttcagagtgtcacgggtcatgttgttagacagtgctttatcaat
tgctgcacgcaaacgcgggttagcgtccggatcaccaccgcccagcttag
ccgcggttaccagctcacgaatgattttagtgaagattttaccgcgctta
gcatcctgcgcagctttacgatgtctggtgttggcccatttactatgacc
tgccataaaaatatctccagatagccctgcctgttcaggcagcgttaatt
acaaactgttcaatcgcctgccggttgctccaggacttagtgagcgccgc
cgcagcagacgcatcaagccacttgtaagccagatgttcagtgaaaacga
tctggcgctcgtgcggaagcgcaagacagaaccatgattccgtattacgc
gtcacgcccggcgcatagcgatgacgtaaatgtgaaaaaatttcaaactc
taccgtgcgctgacagtcaattaaggtcagttgttcagcgacaacatcaa
tggtgacctcttcctttacttcgcgcatggcagcttgcggcgcggtttca
ccctcttccacgctgccggttaccgactgccagaaatcgggatcgtcacg
ccgctgcaacatcagcacccgtttcgtatcttgtgcgtagatgaccacta
agatcgaaacgggacgcttataagccatatcagttattctcagccttctt
cacaacctgaatgctcagctcagccagtgcagtcgggttagcaaagctcg
gcgcttcagtcatcaaacacgctgccgccgtggttttcgggaaggcgata
acgtcacggatattgtcggtgccggtcagcagcatcgtcagacggtcaag
accgaatgccaaacctgcgtgcggcggagtaccgtatttcagggcgtcga
gcaggaagccgaatttctcgcgctgttcctcttcgttgatacccagaata
ccaaacaccgtctgctgcatatcaccattatggatacgcacagaaccacc
gcccacttcgtaaccattgatgaccatatcgtaagcgttagccaccgcat
tttccggtgcagctttcagttctgctgccgtcatgtctttcggtgaggtg
aacggatggtgcattgctgtcaggccgccttcaccgtcgtcttcaaacat
cgggaagtcgataacccacagcggtgcccatttgctttcgtcggtcagac
caaggtctttacccactttcaggcgcagtgcgcccatcgcgtcggcaaca
attttcttgttgtcggcaccgaagaaaatcatatcgccatcttgcgcgcc
agtacgctccaggatggcttcgatgatttctgcattaaggaacttcgcta
ccgggctattgataccttccagacctttcgcgcgttcgttaactttgatg
taagccagacctttcgcgccgtagattttaacgaagttaccgtattcgtc
gatctgcttacgggtcaacgatgcgccgcccggaacacgcagagcggcaa
cacggcctttcggatcgttcgccggacctgcaaatactgcaaactcaaca
gatttcagcagatcggcaacgtcggtcagttccatcgggttacgcagatc
cggtttatcagaaccataacggcgttctgcttctgcaaaggtcattaccg
ggaaatcgcccagatccacgcccttcacttccagccacagatgacgcacc
agcgcttccatcacttcacgcacttgcggcgcggtcatgaaagaagtttc
cacatcgatctgagtaaattcaggctgacggtcagcacgcaggtcttcgt
cacggaagcatttaacgatctgatagtagcggtcaaagccggacatcatc
agtagctgtttgaacaactgcggggattgcggcagcgcgtagaatttacc
tttgtgcacacgagaaggcaccaggtagtcacgcgcgccttcaggcgtgg
ctttggtcagcatcggagtttcgatgtcgaggaagccgtggtcatccata
aaacggcgcaccaggctggtgattttagcgcgggttttcaggcgctgagc
catttccgggcgacgcaggtcgaggtagcggtatttcagacgcgcttctt
cggtgttgacgtggttagagtcaagcggcagaacatctgcacggttgatg
atagtcagcgaggacgccagtacttcgatttcgccagtcgccatatcgcg
gttaatatttttttcgtcacgcgcacgtacggtgcccgtgacctgaatgc
agaactcattacgcagttcagaggccagctttaacgcgtccgcacgatcc
ggatcgaaaaatacctgcacgataccttcgcggtcgcgcatatcgatgaa
gatcaggctaccaagatcacgacgacggttgacccaaccacacagagtca
cctgctgccccacgtgggacaaacggagctgtccacaatattctgtacgc
atgagatatcccttaacttagctgccggcggatgccccctgctgcgcagg
tgaccaagtcgcagcgttagctgtatgtcacaactgaatgaaaaaaggcg
gctattatactggaaattctgccgcaccgtaagagcctggcccgcgctgg
aacgcctcgttaccactttatatcgggcctgaaatcagactctacgccag
tttgctataaaggtgttgcccgaactcataaaaattaacaaaatttgtcg
ttccgccatcggctaatcgcattaaggtgagaggcacgattttgttttgt
caggagtcatcatgcttgaacttaatgctaaaaccaccgcgctggtggtg
attgatttacaagaaggcatcttgccttttgccggaggtccacatactgc
cgatgaggtggttaatcgcgccgggaagctggcggcgaaatttcgcgcca
gcggtcagcccgtgtttctggtgcgcgttggctggtctgccgattacgcc
gaagcattaaaacagccggttgatgccccctcccccgcaaaagtgttgcc
cgaaaactggtggcaacatcctgctgcattaggtgcaaccgacagcgata
tcgaaatcatcaaacgtcaatggggtgcgttttacggtacggatctggag
ttgcaattacgccgccggggtatcgatacaatagtgttatgtgggatctc
gaccaatatcggtgttgaatccaccgcccgcaatgcctgggaactcggtt
ttaatctggtgattgccgaagatgcctgtagcgccgctagcgccgagcag
cacaataacagcattaatcatatctacccgcgcatcgcccgtgtgcgtag
cgttgaagagatcctcaacgcgttatgatttacatcggtttgccacaatg
gtcgcatcctaaatgggtgcggttggggatcaccagccttgaagagtatg
cccgccactttaactgcgtgacgcgggcattttaaaaatcactaaagaac
gcccaagagcatgtgttttctttagtttattcaatgcattaaaaaatagt
ttcgcatgaaattcggtaaacttcatgtgtgcaataatgtcccattcatg
ccccaaaatgccccaaagcagacatttttgccccaagtatgccccacaag
tcacgtcttcaagtcgtctatatccatagcacaccgagttacattcttgc
atccggggtgtcgacaatacctactttattgagtgtgcgagaattaccag
gaacctttccacaatgtagtagtctaatagtcgaatccatctaacattaa
gaagcgttatgatcactagcctctcattgatatcttctgtaatagtcact
ctatgtatcatggtgttcgctacagtaaaggtagggattggtttgtctaa
caatccagacagaaatgataattaacctcaaccacgtaaccacacttcat
acttcatacttcacttaacagtgaagtgctcacatcaccgggcagtcatc
aaactccgcattcctggcatcattaatgatgtacgtgatcactccaaata
tagcgggtgcagaactgtaaccatcatcatctgctggcagcgcttccctt
ctcccgttatccagattaaccaggtgcggctgaggatgagtccgatatcg
cttgatcctgaattccccgtcgattgcacatatcagcagtgaaccatcgc
aggcagtaagtgacgcatccacaacaagcaacgctccctggattatccct
tccctgaaatgtgaacgcgatgcccgcatgaaataagtcgctgcgggctg
actgattagctgctgatcgagggagattcgtgtttcaacataatctgccg
caggtgaaggaaatcccatgtttacgccctctcttgaataccggataaaa
acacagtataaatactgtatatccatccagcaaagaggcaatgagcaatg
ttcgtggaactcgtttatgacaaaaggaattttgatggtctgcccggtgc
aaaagatatcattctgggcgagttaactaagagagttcaccggatcttcc
ccgatgctgatgttcgggttaaaccgatgatgacactgccggcgatcaac
actgacgccagcaagcatgagaaggaacagataagccgtactgttcagga
aatgtttgaagaggctgaattctggttagtgagtgagtaaagattttcaa
tgcccgccacagttacgtattgattatgctgtggaggatattcattttcg
taaacgttggtttgggagaagcggcaaaacggaatgtgggaacaggggaa
aatcagataccagatatgtctgcatttccatctggcaataactggtttca
gttaccaagtggacatatcgttcagatattttccatgaacgttcttggtg
cagatgctaatggcacgtcagctaattaccccattgcttttccaacaacg
atgattgctgtcagtgctctatggtctgatgggactgtagcaaatgcacc
gacatacaagatgatggggaacacgactaacagaacaactttgacgataa
aagtatcagccagctcaggtacttacgggacaatgattattgcggtggga
cgataatatgaataaatacagttactctccttcagaaaatgccttttatg
ctgttgcgttaaaaaatacctatgaattgagtggcacatggccagctgat
gcattagatattcctgatgacatttctgtaaaatatatggcggaaccgcc
acaagggaaaatccgagttgcaggggaaaatggttttcccacatgggctg
aaatacctccaccatcacatgaggaacttattgaacaggccgaatcagag
aggcaattattgattaaccaggccaacgaatacatgaacagtaaacaatg
gcccggtaaagccgctattggtcgtctgaaaggcgaggaactggcacaat
ataattcgtggctggattatctggacgcactggaactggtcgatacttcc
ggtacgcccgatattgaatggcctacgcctccggcagttcaggccagatg
acatccggcgcggtgctggtatctgttgcagtcaccgcgtcaatgtaatc
cagcacggcgttaagtcgggttgtttctgcctgagtcagtttccgtccgg
cctgtaatttcagctgaatcagactaatggaagccattgctgcatcaatc
agtgattggcgctgtgcttctgccgcttctactgaggcaccgtgttgtgc
ctcagtatctgtcacccatttctcaccatcccatttatcatatggcgtta
acggtgaaagcgtgacataaccgtttttgatggcaccgatataatccact
gtaacagctgcgccattttcgattgagtaaacagtctcattgcgatggtc
ttcctcatggctccatcccttacctgtaaatactgccactcttcccggaa
tgttttcgtccgggtcaataccagtggaacaggcgggcatacttacgcca
gtattaatatattcatcagaccagcccgtatattcagacgttactgcatc
ataataaaaacaacgcatatcacccggcactgcagccagcccattttcat
caaaaacaggtttcattatttagccctcaccagaaagttaaatgcaatat
ttcgcggtctgacagcaacaaaattcacaccatcacccacagagttactg
ttgaaattaaatcgtgaaaatcctggctgatttccggcgatgccatcatg
aaagttaattgcgtgtccagcacctccgcctatattcccggcaaactgag
aaaagtttgtagcttcctgccagcttaataattcgcgaccaccatctgca
cctcgcccgtcatcccagacacgaatgaaatcaccgcgggcttcaggtaa
taccagcgaaggaaacactttcgccagcacaggataatcagtggcagaga
atttcgcgccgttgaacttcaaaaacaccatactggaccagctgtcgatt
acagtatttggcattgcagcggacggccagaagaacggaacgccaatagc
tggagaaccttctcccaaaccaaggtttgtgcgagcgtctgcggcattcg
ttgcgccggttccgccgtctgcgacagtaaccgcaccgttgctccctttc
tgcgcaagtttaccgatgcctgggatggttacggcggtgccgttgatggt
aactgtgatgctttggtttgctgaggtggtggcgaacgtctcccacgcgc
caatattctcgtcgtactctttgatgagctgtgacatggcctgcgccagg
ccgtcgactgagatattgtccgacacaaggattccatacttctggccgct
cagcgccggggaaactgctggcgtaaccgtcattgacgtggcgctgttca
cggatgaaatctgaaacagctgcaccgggttagacatcacgataatcgtc
tggccagcgcggacctggctggcgggagctgtccagtttgtgccggagcc
ggttgcggtatttccgttaatagagatagttccggtgctataaatcataa
caactcctaaatttagacaacatgaagcccggagaggtatataaccctca
ccagaaataatttctgaattggtttttaatacatgttgggcaacgccagt
gttggcatagctatagttgtatcaaatgccattgaccacccacccaaata
ataattgccgactactttattcctttctgctctgacattcccacctgtca
ttacaattcctttatacctaatatttccgtaaccaccaacgtgtcgacag
ttagccccggtataaacaatctggcaaaattcatcaccaatattcaggtt
cgcattggcgatatttattgtcccatcaaaaatgaatggcttcgtgcaag
taagaagacgtttgcgttgacgattggtttattcaatccatcaactaaaa
tatcatgtaaacgaattgccataacatttcccactttattttacttactc
aacacaacaagtatattaaaattgaaccttgtcaacaacacaaaggagtc
ccaatgaaactcgctctaattatgctgccattatgtctgtccctcactgc
atgtggtaatggtttaaataccggtaaaccaaattccggtgtcattccaa
aacctttggatcgagatggtaacggttctttaatttatgataccgaaaac
cttccaatgacggggcagtggtgtcacgagattgatcacgaataccgacg
aatcggtagcccttctaactgtgttatagactactaaatattaacccctc
aaaagaggggttaatattttaacctgtgaatgaaccagatccgtgtgata
ctatgatagtaggcgaagaaattgacgcacccctgttattttgagcggac
actttaatacgcactgttacgtttggacctgttacaaccgcagaatgcat
gataagtctctccatgtcttgcacggatgaaccactctcattgccgttaa
tgtcgattgttcctgcaccattacctttaacgtttgctataacacagacg
tgtcttgcgtgcccagaactggatgaatcattataagtcattaccttttc
taaatatccgtcactagaccgactcacattcgtccctgtgtgcatatttg
caatgtccccgataaaactttgggcttccacagtccctttgaatttaccg
cttgttgcttggatctcaccagtaaagctaccgccactagcatatactac
acctctgacggtcacattgttgaattcagcatctccagctttattcaact
tccaaccagcagaaccagctgcatagttgttggactggatatagttaccg
atttttgcgttctcaatggtgccgtcctggatgaagctggcccggatgaa
tgtctgcccgttctggatcacgaacggcaaagctacgctatttccggctg
ccgtggtgacggcgaagcggtcagccaggaagataacctgcgactgcatg
ccggatggcgtattctccacgccgatacccatccccgcggcgtaatactg
cccgttgctggagacaccaaccttgatgttgtacatcgcgctgagttcgc
cattaacgttggctatagcctgagcgttagtggtgatggcggaggtatgc
ccgttcacggtcgccgtgatgccgtttatctgcgtggcggtggcctgctg
atagtcggagagcgtctgattcaggctgttgatggatgccttgttgccgt
tgacgtccgtctgcaggctcagcaatgaacgtgctgtggcttccttctca
ctgacgatcacctcgtcgagacggtccagattcgcgctgttgccggcgac
cgatgcagaaagggttttacgcgtggccacctgagcgaggttggcctgga
ttatcgcaattgcagagttcttcaccccgcccgtcatgccgtccatagaa
acgctgatgttgtcgattcgctggcccagggcggtatcagccgtcgcaac
ggtctgctcaagctgactgagtgaagacgaaacattcccgaccgtgctgg
aaagctcattaacgctggtctgaaccttcccgacgtcctgggcatttttg
gcgatatctttcgcttgctgctccagttcgtcgttggcctgtttgatatc
gttagccatgccagcaattttttcgttgctgtccaccgcgttctcgatca
ggtctttgaacgtttccgactctttcatatcctccagaatgtcattagtt
atttcgctgacatctatcgaggacgtgcccatgatccagtcggtccagtc
cccggcgttaccgatacggtcaatcaggcgcgcgcggtaccactggcgaa
cgccggcaggcatggggccatgctgataatctgcagccgggtacggcacc
aggaccagcagttcaggattggcgtagtcggcagttgtggcgcgctgaat
ctctgtataggccgtgtcgcctgagccatccggaaatttccaggtcaggt
cgatatgccagaccacatcttcggtcgccaggaagttgagcggagtaccc
ggttttcccgttttaccggagagataagttgtttcaccgtatccccatgg
tgacgacgtatcctgcgcattcagcgcccgtacgcgcacgtcatagctgc
ccgaataaatgccctgaaccgagaaaccctgcgcgctggtaaccggaacg
tttatccagtccccgttgtccttacgccactgggcaacataccggattgc
gccctctaccttatcccatgacacgtccaggcttgctacagtcagcccct
gagacacatgatcgctctcagtcaccacgatattcttcggagcagacagg
acgcttatcggcgtgacggtgatcgggggagactcgacccgaacgccgtc
atcgatgtaacgatatttgtttggatcgtgctgaacggccgtaatagtga
aaccgcctgtactgtcgtcgttagccgcgattgaggtgaccctgaagtac
tgtattgcgaggttatcactgtctatcgcccaaacagcgcccgccacagg
aacctgactgaatgccgtagccaccgtcaccgtttttttatcggcgctca
ccgcgctgattgtccgcgtctgggcttttccgtcgggaaggttaaccacc
agccggtctttcgccgcgtagtctatttctcgatcgagggtaatttggcg
gccgttggccgcgcttatacggcccccgttctccttaccagagcggaaag
gatcggcgacaccgataatttcagcgggcaaagggatataaccgtccagc
cccacgccaaacgatacggtcccgtctttggcattggagagcaataccca
gcgaccgcgtcggtgcgcttcactttgcgaggtgcagccgattgcggtca
gggacgtctgccggacgtcgtaacgttctacaagcgccgaatcgtaaacc
ccctcaacggtatcgctgtaatggttctgcggatcggaccaggacaccag
gcaggagctgtagcgattcttgtatgagccgcccgcataagtaaacagcc
catcgataacgtttgagacgttataaacccagtcaacatcgtcctgcggg
acgtctgcctggacataaatctgatcgtttccccagaacgttattccacg
aaataccgcggcgagatcgttaagtacctgccaggcgtcctcctggctct
gaatgaaaacgttgcaggtgaaacgcggttcggtgccaccggccccgtcg
gaaaccatttcgtcacagtactgggcgattgaatacagcgcccacttatc
caccatggacgcatccacgcgcgtgcccatgccgtaaatttcatccagaa
ccagatcgtaaaagatccaggcagggttattggaccatgccattttgaac
ccgccggaccatgaaccagaataggttcgggttttcggatcgtaattatc
cggaaccttaatcagcttgccttttatcttacaggtcactttcggcgcgc
tgccgttgaattggctgctgtccacttcgacatacaggagcgctgttaaa
ggataacgaagcttgctgtcgatgacttccgcatacgaaaacaccttgaa
ggcgttaaccagtttcgaatttgatccgctggcatcagccgtaatacgcc
tgaccctgacagaccagccggacgtggattttggcagatcgatacggtgg
tcacgctgatattccgtcgtggtctttccgtcaaacttgccgtttacaac
cgttttccaggcgccgccgtccgttgataaatcgatcgcatactcggtga
ccgtgcccaccatatcgccattatctttatagagatactggaccggaagg
ctgagcttgatacggatggcatccagggaaaggtttgtaaactggcgcgt
ccagggcgcggtggtggtgacagttgtgcccacggccagctcgttgtcga
cctggggcatcccggcaatataggtctggtcctgtgtgcccttgcggaac
tcccatttcacgccgctgaagttgtattccccgctgtcgtttgccagcgg
cgtatcgttgagaaaaatgttctgagcggtcaggtcgccctgtatttccc
cctcagaaacggcaatgagcatttttaattttgcgaccgacagcagatcg
tcaggctgctcaaccggagtatgtgaactgccacctccccctttggcacc
ctgcaggatggtttcttgtttaagaagctgcattttttcacccataaaaa
aaggtgccgaagcacctttaagttagtggccgctggcctactgctgatcg
ctcgagtacataccggcgctgactatcgctccccctgcctcagtcagacc
gtaggccagggggacaggatgccccatagcgacggtattgaccggcgccc
cgaaggcgtagttaggcgtgttgtccgtgctggaggatttacccgcgccg
aaggatggctggggcgtgagcatctggacaacgccccccagcatcatcga
cactccgacccctgtcagaattgacgtggcgctgatagctgttgcactca
tcgccgcgccccaggctgccatgctcgcaccagcggtaaagaatgcagcg
accagcgcaacagccccgacaactatctgcaggacgcccgaacttttggc
cccctcataaacgggcacgatccggtacacgcttccaccgcgggtcatat
caaactcttccagcccgatattgttgccaccgttaaaaaaggcgaaacgg
atccccttcatatgagcttcagacatatattttttgaatccgggaacctg
tgaacacatggccctgagcatctcgcgcaggtcggcaacatcaaactgaa
cgcgtttaccgaatttttttgccattttaccttcgagaataagcgtctta
accatgcattctgtccttatgcctgaccacccggaccgttctgtcgcgat
aatattttccataaggcgttcgcgaagaaaggtgcccgaaaagatgatgg
agaatgatgttatcacccacatataccgcggcgtgattagtcaccgatgc
ctgcacactcatcatgatgatatctccgggctgcattgcaccggcggcaa
tctcaacgaatccctcacgctcccagttgtcgtcgtagagacgctccttg
ccgctctcccaccattcgtaaggtactgaataattgccgagaacaatgcc
gtattcgcgcagataaaattcacggataagcgaccagcagtcggcgtaac
ccagcacccactgccgcccggcataatcccggtcttcacgcggggaaatc
gtacaaaaatccccgtccggccaggacatgatcccccactcaatccccga
ccagtcgcactggatccggtccagctctgagggcaccagccgaaccacat
ccggatgggaatgaatgagcatgatgatctcaccgcgcgcgcgggcagcg
agctggtcttccggggagagcgtgaatgtctcctcgggtttatcggcaat
gttgcggcagggaataaagatttgttgctggcctgactgaacaatcaggc
cgcaggcttctttggggtattcagcagcgacgtgctgacggatagcatcc
agcaatttttcacgcatttttatttcccctgcaggtttgcagccggaaaa
ccgccgaacggcagcggcgcatccgggccgtgacgatcctgacaatcctg
ccggcggccgccacaaacatctttcgacgggtcatcggtcggtgtaccgt
ctttggtaaagtatttcgtgccgttgtaatcgcatccggtcccgcttcgg
taccagccccgcatacaccaggtgcagacaggcgtaatctgccgtgtcgg
cagctgcaggctctgaatatcgaaaggagaacacagctcgaaatcaacct
gtacccgcgtctctgcggttttagcattgacgtaaaagagctgtaagcgc
tcatcggccgggctggcacccggattaccgtttttccagttggcggcatc
gagatacttcgaaagcgtggtatggattttgaccttagccctgaccatat
cgtcatattcaagacacagcgcggtgacatagtttccgacgttcccgacg
gacagcgtgggcgttggctgggaacctgtactcgataactccatcccctt
aagttcgtagggatggggatcgtactggtttccctgccagataatggcgg
gcagattttctgcggcgaaggctgcccacccctcttcctgaatattgtgc
gcatgaaaacgcagcacctgatccataccgaattcagtgccgtcgatctc
aatcagctgaataacgctgccgggctcaagctgttgtatgtctgccgtaa
aactcatactccccccataaaaaaaagccgcccggaggcagctttcagtg
tttttcgagaaaatcagggcgcgaacgcctgttcaaaagtgaaggccaca
gtggcttttttcccggtagggaaagaaacgctgaacgaatcggccttcat
tctgaacagctttttttcaccccatggagtggtccaccagaacgatttag
taacgtgagacatcaggaaagcgcgcagcgcagccgcctcctgtctggtg
cccgtccagtccaggttccacgtttcctgtttgtcgttgatccccatccc
cgctatctgtttgtagccatccccgaactgggcctgcagcgttcgggctg
tttcagtgccctgcgctgtttttcgcgtgcgccaggtaaacgtgtccgtc
actgtgtcctcctcgaataaagcacgccgcccgcggacatttcttttttc
agtcgctcggtgattgtctgctgaacaatcgcctgcagctgtttcgccgt
ccccgtggcgttcgcctgatttatgcttccgtcactcccctgctggctga
tgctgactggggcataaacactgatcccgcccatgccagcaccggctgcg
ttcccgccgccgaccagaccacccgaggcatacccgcgcatcaggcgata
gagattagccacgccgatgcggctggttgattctttggtgaagacgaatt
ccccgcggtgaacgataccggctggctcgtacttgccgccgtgcccggta
aaaccgcccacgtcaaaaccctgtggccggtatgacgggaccgcgaatga
ctgaccggcagaggaggttttcgccccgccgctaacccagcccattgcac
tctggatggtgtaagccaccagcagctggttgataacggacacaatcatt
ttaaggatcgagctggtgaattccctgaagctcgccttcccggttgtcgt
caggctggtaagctggcccgccaacccgctgaacgtagcctgagaaatct
gctgaacggagctgaaaacgtttgtcgctgaatcctgatattcggcccaa
ccctgtttcgcaccggccagccagtttgcacgcagggcatcttcagcttc
gaacgtcgccctttgctcttccagaaccttttgctgcgcctgagggttgt
acgaatagctttcgctgagacgctgcagcgtagtttgtcgcccggcttcc
cgggtggataacccctcagactgagcctgcaggcccgccctggcggcttt
ttgctgctgctcaaacttcacggcctgatcggccagctggttgagctttt
gctggctggcaaccttatcgcccaggtcggccagctgccgcttgtactcg
agcgtttcttctttgtgcgccagcagggatttttcctgcgccgtaagctg
acgacgcccagcggcctcctgcagaacggtgaactgattttcagtttgcc
agagatcctgacgctgtttacttatgacgtcgttcacgctggtatgctgc
tcaagcgttttaagctgggcctgaagggtgagaagttcggcctgcgcctt
ttcctcggctttgtccccggcgggcgttgagtagcttttgcctttcggtg
tttttggatccttccactgcttttcaatcccggcgcgggccgcggcaatg
tccttttcagtccacagcgtggcgacaccgtctttcgcatcctggcggtt
tttctcaataagctgactgagctttttctctgctgaagcccgcttttctg
ccgccgtcgcgccggactccaccagctggttaaactgctgctggctgcgg
attgcctgagcctgctggtccgttcgcattttttcccgcgcggctgccag
cccttcctgggcgtattgctgatcggcaagatcgtaagcctgctttttca
gctccacctgctggcgcgcgtttctcagcctttccgcatccgctttctgc
agaacgttgttaccggcataatccgggtcgaccttaagattgctggacag
cgcgcggtactctttctctgctgcctgccactcagcaaaagagtcctggc
gcttcatcgcggtgtcaggattacgcccgacgcccagcatcgcatcccac
gcaccggaggcggcattcttcacccagttccaggctttttcgagggatcc
gagattatcctcgaccgcaccggcgcgctgaatgaccgcgtcggaatatg
cccgcatggccagctcggcagccttctgagaatcccccagcgcctgagca
gaagctatctgttcatactgggtggctgtcagaaaatgaagggaatcgtt
gagcgtcgcgaccgcgttaaccggatcatccttcaggcgtttaaactgat
ttatggtttcgtcaacggcctgcccggtagcctgctgcagcctggcggca
acattgctgaccatgctgacgtcattaccgctgaacgcgccgctgccaac
gacctgcgccagcacgcctgcagcggcatgctgagtgatgccattacctg
ccagcgagcgcgccagcgcctgcagctgccctgacgttttccccgcgtag
ttcccggtcaggatcagctgcctgttaaattcctcagactctttgctgcc
gtcgtaccaggccttacccaacccgaataccgccgcggcaatccctccga
ccatgctggcgatcccaagaccgcgcagtgacagaagctggtctatccac
cctgcccggttagccagcgtgatcccggagccgcgcagcgcgccgaagtt
accgcgcatgacctcgccgatcagtattcccagttcctgccgggcggcgg
cactttgcagccccagaccgtgcgtggcgactttggcagcttcgagcttg
cggatatagacttcagccgcatcgctggcaccgacctgcgccgccttcat
gcgcagtagctcggtaccggagagcttttgctctgcaacctgttgcttca
gctggctgaggaatcgcgtgcgcgctgcggccgatttttcctccacgatc
tgcagttctttttgacgggccgtggtgcgggaaataagggcgagataatc
ctgctgggttatgttgccctgtgccctcgctgcgcgaaagcgcgcctgca
cgttcgcaagcgactgtgtttcaccattgagctggcgtacgccgtcgatc
tggcggaaaaatgatgccgcaagttcatcctgtcgacgggcaagcgcagc
ggcctgcccgtcattctcacgcatgcgctgattaagctcggtcacgcggc
ggtgagtttcatcaacggactttgaaacgttctgccagtctttggtaagc
ccttccgttgcggccgactggcgggatttcatatctgcggcagccgccgc
gccagcgtcacccacggttttaaacgcagccgcctgccgctctgaagcgc
gctgcattcgcgtctggactttttcagagtcctcagccatcccggttagc
tggccctttatgcgggcaacctgctcactaaacgtggcgctgtcgacgtc
aaggttgatgaccagatcgctaatctgctgggccatatcggatacctcct
gttatcccctcagctgcggccatcagcgcatcatcatccggctcgtcatc
gctgatgacgataccggaaggagaaagcaggctgaaatgtgcgggggtaa
gttccgggtcgcggaagaaaagagtggagatggaataaagcagctctgag
aaatgcgcatcgagctgagcgtcctgaaaataatgctcccggtagaactg
gtgccagtcgcccagctcagtggaagtcattccagccagcatggcgcgcc
agtcgggtcgcccgaactcgcgcgccagattcaggacaaacttcagctcg
ctggcaagggcttttccgccgcaacgggttctgcgctttcggcctccgct
ggggcatccggatcggcagctttgtcatcctcaaccggaacgagcatgcc
ggagagcagctttatttccatttctgctttaccgatcgcctccggcggcc
agccgctaagcacctgctggtaaagcgtctccacatccgtgccagccgga
tcgttatgccacaaagacatcgcaatcaaacgcgcaccgcagcgaatatt
tgagccaatcagcctggccgtcatttcctgatcgctgatgccgtcgctgt
cagcgctgacggccttttcctctgcggccataaacgtgatgtactcaata
cgctgaagcgccgacagctcgaagatggtcagtgattctttttgccaggt
gaacttctcttttttcagaaacatgcgtccttccttacgctgcagttacg
gtaactttgcagaccgcaacgaaattaccgtcgctggtcataacaataac
gtcagcggtgcctgccgccacgccggtgacggtgatcgcattaccgctaa
cggtgaccgttgcttttgccccgtctgaggttgccacacggaacgaggta
tctgaggcactggctgggttaaccgtcacattgagcgttgtggttgcgcc
gacggccacgcttgccgtggctttatcgagcgtaacgcctgtcacgggga
tattcggggtcccgctttcttctgccagttccggcttgccggtattggta
attttcgctgtacgggttatgacctcttttgccggaatggctttacccag
gctgctgcaccagccgcggaaaacgtcgacggtaccgttcgggtatttga
ttttgtaatagcgtactgagccatcaataaaccatgcgacaaggtctttt
tgcccttcttcgcccggcttccaggcgagggtgaacgaggtatcgccagc
agattttgccccctgggccgtcgcgttccagtcggcatcctcgtcgtcga
ggtaagtgtcgtcatacgattcggcggtcatttcgcccggcgtcagctct
ttaattttcgccaggcggttccagtcgatatccgagagtgggttagcgaa
agcgttgcccgttccggtgtaaagccagagggtggtaccggcacctttca
caggggccagcgggtttggagtaggcataagtacctcttaaattgaatag
gtgattaagtacgtgaaatcgactgaaccccaggtggccatttcatcatc
ccgctgatagtcataaccctgcggggtgaacgtctcgaccagttcggtca
gacctgggatgaaggccattgccggatacactttctcttccatccaggaa
tcaagcgcgctgtcggggctggaggctttaagaaatacctcgatgtgaac
aaccgcctgccacgaatcttcgtcaagcgaatcgccggtgtactccgcgt
cagaaaggtatacagccacggcagggagatcctgctcttcaagaaaaaca
gggcgcccgtcaaaccaggtgaccgtgtcggtgatctcggctttcagttt
ggccagaatggctgcacgaattgcgctgtgtctgttcatcgcttcaggtg
gatcctcagttggtttttcagggctgcggaaagttctttgggcatatcgc
tttcaataaggcgctttgaaatagcggtgaaggccacggtgagcggtgtc
tcaagaggaactttgaccacatcaatcggataacgggcctgacctacgcg
ccgcatgacctgccagcgcccgttcgcaagctgttggataaaagcgttac
gaaaggtatagggcccgattttaaggacgctgcccgctccgtttctggcc
ccttttttacgcgagagcctgacgcgcgccgtgccgagctttatcgcagg
aagattaccgcggttgatttttatcgacgcgaccgggcgatcgtgacggg
ccttgcgcagacgggaacgctggcggaccagacgaaccggaagccccttt
ttccggttatcatcaactgttgcttctttcgctacagctttgctcccctg
gcttatcgttcttctggccaccctgttaagtgcttttgcggttgcctcag
gaacgattaaccggctgaggctgttcaggttctgaatagccctttccagt
cctttcacagacatagcgcctcctcattcgagatggatgcggggttttcc
gttgaacatgtcatagcgggtaacgatcaggttcttaccgtcgtagtcga
cgctgtcgtttcggcgtggctggtaaagctcagagaaaaccaccagcgaa
gtacctgttcccgacaatggccccatttcctcgagttgctcggcgggaac
aacgtcatagctgctgccattgatgatcgctgtctttcccatctttttta
tagtggccgcgtccatgcgcgccgccatccggtcaaaggagttaggcatt
gatcttaacttcaacaacggtggtgtttgcccctgcatcttcccaggcga
tgcccgcggcaacggcgtccgtttcttcgatcgtgattttgccgtccttc
agatacacctgcgccccggcagtaaccgcatctgcggatacttttggcag
gaggaaaacaccctcagtaaaaccgtccccggtatcgccagccgggatat
cggtaattgccaccgcgataagttttccaacaacaaccgggtcgccgctg
tgaacatcggttgcaccactgtttaccagagggatcgttttcccgtcctg
cgcatagttcttagccataacttctccattcagcccctttcgaggctggt
ttcaggtataaaaaaagcccttacgggcgtctgtttgtcaggactgtttt
ttactgaccagaggatttggtcatgccgcgatagtccagcggcgccacgc
cagcatcaatacgcactttcgtggcgataccatcagtggtgaagccttcc
tgctgatcgatgtatggcgtgtcgacgccgttgagataagcgacctcaat
ggtgtcggtgcccttcgcggcagccagataccaggctttcgcatcagctt
catccagacgtggttcggcaatgacttctgcaaagttctggatagggtta
acgatcccggcattgatgtctgcacctttaacactggccgacttgatggt
ctgatttgccagagtttccagggcgacgggcaccagcatgtaggccggac
ggatattcagggttcgctccccctccttctgcagacgcatcagcttgcgc
gattcgtccaggctggccacagaaattgcacccgagctcaggttcttgtg
atcggcatggaacagcgcctttccgtctgagagtttcgggtttttggtca
gaatggcgtaaaccagatcgccaatcgttgattcgccgcgcgccccatct
tcatcggtacgtcggtaagctggttcagatcgtcgttgatgatcgcctgg
cgagttactgagaagatttcaccatacgtggcaagcgcgatggtttcgcc
tttgtcactggtagtgatgtacttgtactcagccccttcgcgaacctgtc
gcagagaagggaacccacccataccgacacgatgcgccgttttgaagtcc
gacagctggccttttttggtccactgctcgaaggtttcctgcgcctcgtc
ccagccctgaatcagcgctttgttcgcaacatcaagcagaatgttgccaa
agtcagaggtgctgtgggtcagcgccaggccaaccatctgcatcgggttg
tagctggccacgccgatacctttttctgtcagggccatacgcgcatactc
gcgcagcgtcataccgttataaacgttatcccgctcctgaccttcgaacc
cggcacgcgccatcagtgcctggcgaataccatccgcgacgaagttaccg
ttgcccgcatgaatatgcggctgagtggttttattggacggcgtggccgt
tttaccgagttctgccagcagcaaatctttcgccttatcgacggagcaat
cagggtcggccacacactgattctgcagttccatgtgcttattaccgaac
atggcaaagagatcgccgatagcgttaacacgggttttctgctcagccaa
cacctgcgcgcggatcgcattttcatccggtgccgggtctgtttttgcct
gcggtgcctgaggctgggtaataaccgggtcacgctgggtagtgttgcgc
ggcggggtgatcatgttgcgaatgctttttggcattttttcaaattcctc
aatacgttttgaatgaatacaggccatagcctgaagggatggtgtcacct
ggtcggcaaaacccagttcaaggcactcgctgccgttcatccaggtttcg
tcctccagcattaccgcaatttcttcggtggattttccggttttctgtgc
ataagccgggataagaacggattcaaccttgtcgagaagatccgcatagt
cgcgcatatcgctcgcgtcaccaccagcaaacccccagggcttatggatc
atcatcatcgtgttttcaggcatgatgaccggattgcctaccatcgcaat
caccgaggccatggaggccgccagaccgtcgatatgtacggtaatcgccg
cgccgtggtgcttcagcgcgttataaatagcaattccgtcgaagacatca
ccaccgggcgagttgatataaaggttgatgtgggtgacgtccccaagtgc
ccggagatcattgacgaactgtttcgccgttacgccccagtacccgattt
cgtcataaataaaaatgtcggcctcactgttattgctggcctgcatgcgg
aaccacgaattactttttgcgctggctttcggacggtggcgcgcccggtt
ctttggcttcggcactggtgcctcctttatcattggcggggtcggtgtca
aacaccaggccctgttcacggttctcgtcaacctcagctttacggcgtga
cttaacatcatccgggttgcgaccgctggcacgtatccagtcggattcag
tagcagcaccgccgcggatctgcgttttccaggcattcgcttctttaacg
ggatcaatccacggcataacgggccccgaataaaccgcgttataaagcga
gtccatatcaatgcctctcggcagcttgatttctccggcagcaatagcca
tcttgagccaggctcggtacatgggccgggtcactgaaccgatgaaccag
tcctgaagaatcagatagccgtcggttgactcgacaagctcctgccgctg
ggcactgtacgttccgttgtagtttctggatgtgctggaaaagctgaggc
gactgccggcggacacggcacgcagctgtccgttacgaaaagattcgagg
ttagggttcgggcgatcggatttaatcatcccgatttcttccccggcctg
cagttcgtcatagagcataccgggctgaatcatcagctcgcggtcatcgc
tgcttgaatcagaatcgaagctctgtccgtcgccttttttgatatacatg
ccgagtgccgcagcaattctggcagcagtaagctccgagtcctcgtattc
tttcagcgcgctcagacgcatcagaacaccagacaaaagagacgttccgc
gggtctggtgcaggcgtcgggtgaatttgagatgcagcatgttctctgca
tctatctctttggtatcaaactgacgcccggatactggcaggcttttata
gacctgatattttttcgggcgtccccagttatcgacaaaaacgccctgat
tgagctgggtggcagcatcgctgttcatcggcacaaagtccggctccagc
gcttccagccagaacggcacgccagcaaccggctgaagaccatttccggt
accgcgaaccagctgagcaaatacctcaccgtcccggagccacgttcgca
gcatcagccgctccagcattgggcgggtaaactgggttgtgacatctggc
cttacggaccattcgccccactttcggcggatatcagtggccagcttttt
agcgatcttcccgttactcagcatcggatgcggttcaactatgatgccct
tcgcacccaccaccctttcttccagcttgtcgaaaacgccgatcaccaga
tcgtggttgttatccagccagcgcgcctgctgcctcagcgaaaccgcccc
catctggctgagctgatcggctgaacgattttccttctgggctttgtggg
tacgcgtttgctttaccgcctcatacgctttaataactgcgcgggcacgc
aggcgtgaggctttccagcctggtgaaaacaggccaatcgcatcatctaa
aaaactcatccaaacctcgccagcctgtagccgggtcgcccacggcgttt
gttattgagcgttgccagtcgtcgctcccattcctgacggccttttctga
tttccgacaggttttcgagcgtcatctgctgcccgttgaaagtgattgat
ttcccctccagaacagacagctcggctgcagcatagcggtcgatcatgtt
ttgaatatctgctggattcacacccaacctcctgacgaagaccacggatt
agcctgctcggttacgggcttctcacgttttggttttggtttagatttcg
gcgcaggcggcggggatggcatttcgccagcttccgtctgcgtgtcctcg
atccacgtttcccgccgtgcccactcaggagctgacggccatttgatttt
ttcgtaaccactaaggatggcgagcgcgtcggcataaacgagcaggtcaa
atgcttcgtttgcgccccggccgggcttactccatttcccttcattcgag
cgttcctcatacgtcagttcgtcatagaaccagctgcccagccaggcggg
gaaatgcacatagccagggccgggtgaatcacgccacagcgcattattca
cccggtctttaagggcatcggtctggagaagataaagaggcacatcacca
gtcgcctgtgcgcggcgcgttgatctgcccgtgttgtcgggaaacgttcg
ctggataagtttgctgcgcctgacgctgtcccctttgaagagatagatac
gcttacccagcccctcacggcgacatctgcgccagaacttgtaggcatta
tccgtcacgccatcttcgccccctgagtccacggccatcgacatcagccg
catgccctttgatgggtcagctgcgagcggccacgttttatcaaagacgt
cagtgagtaaaagatcccagtcctccggatagctcgccggatccacctga
atgctttcaccgttgccgtcgcagcgcagcgaatgccggatgttgtaacg
gtcaactatccagcgctcacccatacttccataacccgtaatctgcacaa
caaagcgccggttgcgcccggcctgcacgtccacggtcgcagtgagaaac
tgcacgccgttcggtaccgaacgttttgggacgtcttcggcacgctgctc
gagcaattcacttttacgctgctccatgctggctcgcggcaaatagggcc
tgccgaaatcggtgttgatcaccgtcttcagggtttcttcgctgcgcgtg
gattcatattcctgctcggcggtcagaaacttataaataagctgcgccca
ggtctggtaagcagctgccggaccttccatccagaaggaggcaatacggg
aacgacggccatcaccgctaaccaggcctttcctgtcgatggtttgcccg
tcccggagccagacacatttcatgttaagcgcacgcttcatgtccggtgt
gatcctgcctttacaggcagggcactgaagaaacgccgcttcgctggcaa
gcacaggatcgctgctgtcgcggtatccggtcatattgtccatttccggc
tggaaatattcgccgcaatgcgggcatggccagtaaagacgacggcggtc
accacggttatagagcgataaaattccggtggtcggaggggcttcatggg
gcgtggagcgccgccattttgtgtctctgatatccctcccgggcgagctc
tcaaccagcgtcatcccggaggacatgaatgtcgtggttcgtttcgatgc
cagtgaaaaagcatccccctccccgtcgatatcttccggaaagcggtcat
aatccgtcagcgccacacttttatagtccgaggacgacatgatattgacg
gatggccagcccagcttcagatagttaccggcgcggaatgtacggtcgta
gacgttgttatcgttacgtcttgggcttagccgggttttaacttcagggc
tacagcgaaaagtacggtccaggcgttttttggaatgctcgcgcgctttt
tcctcagatacctgaattacaagcatatctgccggatcgcagacaatgtt
ataaacgatccagccgtcaatcagcccgatggttttacccgttcgcgctg
ggcccacaaacacaaccgcatcgtattcacgcgatgccagacagttcatc
ggctcaatcacatagggtgccagatccggatcccacggaactgagtttcc
cgcccccattggcacgcgcatataagtactgaccgcatcggccaccggca
tacgacgcggggctcgtaaaataccggaaacatcgcggcggatgtccctg
gcggatgcccgctttgccatcagtcctcctcaggctgctcctcctctttt
ccagcgtcctgcaccttctccgccatctggtcgcgcagatcatcgataac
gctttgcacacgaactaccgcagcaggcgttaaagcacagtcgcgctcga
gcacatccgggagggtttcaagtaccatgacgacggctttcgccatcaat
gagaattctcgcgccacttcatctgcgggtattaactgccccgtatcctg
ttcgaacttcagcctctcgttctctgctttccagtgggacagcctgtcag
aagggggcatatcgtcgatgttggccgaaacggtagggatcatcagttcg
gtcagaatgtcggtcaccagatagagctttaacttgctattgctgcctgg
agcaggttcaacatttttcagtctcgcggcaaccgtctgacggtgtacgc
cggttatccctgccagctggttgatattgagttttaaagtggcaatttcc
tggtccatgatggtgaacactttttgaacgattcgacatgttgcgaaaat
ggcctctaattaaatcaaagacctgcgcacatgatgatgatgaccctgga
tccgaaaaactagccgtttcccgcgagcacgccgccccgtggcagggtcc
ccctccgggagtaccttttgataataattatcaattgcacactatcgacg
gcactgctgccagataacaccaccggggaaacattccatcatgatggccg
tgcggacataggaagccagttcatccatcgctttcttgtctgctgccatt
tgctttgtgacatccagcgccgcacattcagcagcgtttttcagcgcgtt
ttcgatcaacgtttcaatgttggtatcaacaccaggtttaactttgaact
tatcggcactgacggttaccttgttctgcgctggctcatcacgctggata
ccaaggctgatgttgtagatattggtcaccggctgaggtgtttcgattgc
cgctgcgtggatagcaccatttgcgatagcggcgtccttgatgaatgaca
ctccattgcgaataagttcgaaggagacggtgtcacgaatgcgctggtcc
agctcgtcgattgccttttgtgcagcagaggtatcaatctcaacgccaag
cgtcatcgaagcgcaatattgctgctcaccaaaacgcgtattgaccaggt
gttcaacggcaaatttctgcccttctgatgtcagaaaggtaaagtgattt
tctttctggtattcagttgctgtgtgtctggtttcagcaaaaccaagctc
gcgcaattcggctgtgccagatttagaaggcagatcaccagacagcaacg
cgccacggaaaaacagcgcataaagcacttcattagcagcgccagatagc
gtaatgattttgttactcatggaatatttccttttaggcgtgagcctgtc
gcacggcaatgccgcccgagaggtaaacgcaacctaacggcatcacccag
gctcactactgaaagactctctttgatgtgcgcgtgcgatgcgcgtagaa
gactgatttatcaacctgtctttatatcaggattcattacctgactattt
gtgggtaaagttcgtagtgcgctgatcgtgcaaaatgattttagttggga
acagttcgcaactctgtcccataaaaatcagcatattcccatctatccca
tatccagcgcattgaccatcgggatactgaagggagattccatcatctct
tagaaagatcaccatctcttttgtttcaatttgcatatagctacctggag
gatttatgaatgcaaggattttcatggactattaccatgagattgatttt
ccatctttattcgcgagagcagtggaaagcgatgacgatgtgggtactac
attgcgcattcacctactttgtgagcgcatggtcgaagcatggatatgcg
catgctgtgactgccaagatctctttggaagagataaaaacaaactttta
atcgaatgtaatactaaaatatccatggcgggaaacctgggaatcccccc
ggaacttatgaaatcacttaaaaccatcaactcaatgcgtaatgaccttg
cacacaatccatcaatacaaagcattgctgattcaaggatccagagcctg
aaggatactctgactgaatactttaaacagcatccaacggaacccagcat
ggaagaatcaaaactgggtatttttaacgccgagaatcaattaaccgaag
aagtttccttagatagtgacagttcaaaaaacagacttaagttaatcttg
ctgttcagcaagttaatgcaggcgttaatgcaattagttgcagctaatca
taatgggcgctgggataaccaatttagccaattcgtttaccatgtgacca
tgaacgcaacaaagagataaatccaagcccgttttgtacgggctgttgca
ttatcacaggcactcagtgaatgcctgctgtaatgccgctagtcgtcgag
ttgcaacacaccgtgatccagtgattctgaataggcgataagtccggtat
aaccggggataatctcaccattatcagcttcaaattcaggaattgtgccg
gtggtgatggtgtattgaggctggccatcttccttcgcgaaggctgccag
gtcttcaatctgcttagctgtaagaactactgtcatgctcattcctcagt
tgtaaaaaagccccgcgagtgcgaggcgatttgattgaattctcggctct
tatctcagcgcagccccttactgcgtgccggttgctcggtgatgagcatc
agcgatgagacattaaagccgaccgaaggccagcggcgttcctcatgttg
ccgacagagccatatcgacaagaggacgaaaactagcagcatgaatcgcc
tattggttattcgacagtcgcactgattcgtaaatccgctcacacgtcat
tcctgcccggtagctttcgtcagatcgtccagcataatatcgagctgctt
ctgcaaggcttccgagcatgtcggcaagcattgctgcgttggctccggct
gttttgcttctgacggaagtggcgagatctgcggtgtgctttgcggcgtc
catgtgggtagcgagttttgttgcttcggcgcgcagctgcttaacagtgg
tagccaggccagcagaagtaacggcagcgctcgctgcttgagcttgagca
tctttaacggcctcatcccgggcgattgttcgcccttgttcaatcatacg
agctgcggtctgtgcattcgcttcctgtgaagattccgcgctatcccggt
cagcccactttattttccagctgcggttcgtccactcactgccagcgaga
aacgaacctaccaacgcaacaatcacaatgatgcagatgccacccggctt
cactggtctatcccccagcacgtcagtgcgctttcctggtcccgccgttc
tacctgcccataacatccatccttctggcccttggtcaggcggcaatcgc
ggccaccgtctttaatccaccagcggatagcttcacaggctcctttcgta
tcgccagcattaattcgcttatagaacgtagacgggaaacattttccggg
gccgatgttatatgggcagaaagaagcgatacccgctttctgtggttcgg
tcagtggtactttgatatttcggtcaacccacgccagcgccttgtcgcgt
tcaatggcgtttacctgggcgcatttctcagctgacagcttcatgccctg
tactactggcttgccatcaaccattgttgcaccacggcaaatggtccaga
gtccgccgccgtcgcgatatgctgtcaagctgttaccctctttctcatcc
agaaactgatcgagaatcacgggtgcggaagccccggcaagaatcaaacc
aacgaccgctgcgctcaatttattcttcagctttagagacatagccattg
cgccgatcctcccgttctttccagcggaaataccagttcactgcacaggt
gattaccgtgcatgcgataccgacaataattgcccagtcgctcaggctta
accctgcaattctgtcggccaacatccaggacacctcttttgctgtttta
gctgtttcggcatatgccttcgctgatacaccgcagccggcaagcgtggt
tcctgatccatatgaaagtctgctgtaaatggtgctcattctggtcatag
cctcacctccgatagttcggatggcgctgtgtgtgattgaaggggatcag
gcaaccgggctcttatgttcaagtaaaaattaaggatgattcccggtgcc
tgaagatggtgatcaccacagcaacgggggagcgtggtgatcgttatgat
tttttcagtttttccacctcttcggtggtctgtataaacctgtctgcctc
cagttctacgccgatcgcccgacggccaagttctattgcagctttcacag
ttgaaccagagcccataaagaaatcggcaacgatatcccccggtctgctg
ctggcgctaatgatctgtttcagcatgtcggcaggtttttcgcatggatg
tttgcctggataaaactgaacaggcttatgtgtccatacgtcggtataag
gaacaagagcggaaacagagaagcagcgccgaaggattttgtattcctcc
agcaattctgaatacttgcggtttaatgactggtaggtagccaccagctg
gtggtgaggatgttcaagcttttgctgaatatgtttatcgatggcgatcc
gcgtgaacagttcctgcaattttcgatagtccacttcattcggtagttgc
cattggcttgcaccaaaccagtgtgacgccatgtttttctttccggttgc
ctcagctatttctttcgagctgacacccagtgattcacgggcattacgga
agtaatcaatcagcggcgtcataatgtgctgctttagctctgtgcttttc
ctttcgtaaacatcctctttacctgtatacggtccaagatagtgctcagc
aaacaaaatccgttccgtagatggaaagtacgcacgcaggctttctttat
tacatccattccagcggcccgatggttttgcccaaatgatgtgattcaaa
acgttgaatcgggcgcgcatcataatctctatatctgaggccagtcggtg
accgcaaaacaggtagatgctgccagcaggtttaagaacgcgagcatact
cagccaggcagctatcaagccagcgtaagtagtcctcgtcccccttccat
tggttgtcccagccgttgggcttcactttgaagtacggaggatccgtaac
tataagatcaatagagttatccgggagggtggcgacgtaatgcagactat
cagcgttgattaactcaacactgtttatttttacagtatttttcatagat
cagtaagcgtaactctgataggctcacgttgcttttgcgctaaagcagtg
ggccttggttagcttgtgacctgaaagcatgagctgatggctggccgggt
gcgctaacacccaccagccgcccatttccacagcagaaaacccccattac
tggaggcgtttataacatccgaactggtaatcagataaccccgccatcac
cagctgcgtaagtatgagctggcaacgttcgtggctgaggtgggtattct
gtgcaatctccccagccgttgctggtttatcgcttaattcattgaaaaca
gcctttgccgtttctgtcatatcttcctgatttagcatgtcttttaccta
aaattagttgcgtgacatacagataactctggttggtgataccagcaaga
gaagaatttgattctgcaaccaacaaggcctttaggcatcaggcaggaat
gagatgcaataaaaaaaccacccgaaggtggtcttatatgaatctttaac
gcggacttagcaaatattccacatcatcgtactaccgttatggttttcga
taatttttgcggctgggctagtaccaaaagagtgcatatagcaatgatga
atagtaaggaccagatcctgcaacgtttggtcactctctagctccatgat
atttaaaccaatattttgagctttgtccaaatgaatatgtctggcatgtg
catacgttgcttggtggttgtttaactcatcacatatacgcttagcctta
gcttcagcgtcagcctgacctgcgaacataccagtacaaagccatttctg
gacaatttcgttcgcccagagaattgctttttcacactcgccaatcaacg
ttggatttagtttttggaacgtaaattgccaccattgcagtgcagcaggg
ttggcaaaaatttccgcttttgctctctcatactcctcaataattgcatg
agatgataacccattaaactgtggatcaattggccccaagttcgactgtt
tacctaaaacgatctgctcagcacaacaagcaagcattgtgccacaactc
attgaaatcataggtacaatcgctcggatattggttccgaactttgaacg
aagataatgaccaattgattctagagctgcgatatcgcctccaggagtat
ggagtaagatatccaatcccagactcgtatctaacccattgatagcagac
ataagaccatttttatcatcatctgacatctggatcagatgttgaaaccc
aggcccccctttttgaaggaagcctgagtaataagaaattacatttcggc
cagtatgtttcgataaatcacgtaagtacttgtggcgaacctcatccgct
ggtgtacgttgagcgatagtacccatctcacccaatacgtctatccaatt
tggcatgttatcaatttatcagtatgagtacagttggtgagattgctgac
cgttctgctcagtagtatttggtgttactgtgctgtatgaatagagcaca
ccacttctcacattcagatcgttttgctgagcgagaacacgcatagcaaa
atgctgtacggattcgcctttttgaaactcttggggttgtatgcccattt
tttcgtaaaattcagcagcgctcatgatatgtccctcgtttttttctaca
tctatgcaattccaggagccatcaacacaagatgtagtagttagcagtcg
tcaaatacacgaaaagcctcaagatgaggcttaaaaagattctttttgat
aaagatttagccaaactatagcggtcaaaatgcagatttgacaagtataa
aaagcacttaaagcctataaataggcagtttttgagaattaaagcatctt
taatgaggttgaacaaaatgcagtcttgacgctgaacaggactttactgg
aacgtagagctaaatggttcgatttcatgaaccagttacaaaaaaacccg
ctcatcggcgggtttataaaactttggcaacatatcaaatatgcttcaaa
tatggcttattttgttgcattttgcaagcgtgtttgaaggagatggtgaa
atttacttcacatttctgccactttgagggcttcttcttcctcatagtat
tcaagagccatggccaacgcagattcatcaagctgggtaaaagcggcctt
taacccagcccagtgccctgaatagacacgcaaccatgtcgaacggtcaa
cgctaaccatgcgggccaacgctgcaccagcatagtctttataggtttca
ttatttctggttgcggcaatttcctgccctgccagccataccaggcctat
cagtttctttactacgcgctcctgaagggagttatcacccaggcatttct
gataagttttccagacgtattcacacatcatcacctggtgcttatagcta
aggtcaaaaccgtagcagtaccgcaaccaggcctgctggtatccactaag
cgcggacactgccctacgccacggcgcggactcaaattccgcatctttta
tcggcggcattggcctgcggcggctgcgtgtttccagcacatacagtggc
gcggaaagtgagttaacaaagcgtggccccttctctccttcgagttcgac
gagatgaattccacggcgaggggtggcatttttgtctgctggtgggtgtt
cactgaaagcctcaagctgcccttttgttcccccagacaggtcaggtagc
gcgcggcgcaattctattcttacaaaattcaggtcttgttgattcatgct
tctttgcgctccatacacttaagctttcgcaattacgccgatcgccagcg
cccgatccataaaacgcagtagcagctcaagctgcgtaccatgcttctgc
tcgaatgccggtacatcggcgtgtaactcgtcgtggcactctctgcacag
agggatcacgaagagatcatgggcttttgttgctgtcccccccataccgt
gccctacgatatggtgcggatcatctgctggccgtcggcaacactcacag
ggttgtgttttaacccagcgggtgtacgtctcatttatccagcggcgtcg
ctttggcctgagcatgaaagattctggagactccggatcaacagagagcg
tgaggatcttcttcgccttctcctgcacgaggctggttgcagaagcggaa
ggcacaatgtcgctttccctcatgaccgagcggatcttatcatccggaag
gcgtagccccttgtgcgcaacgctttccggaataacatcagccaggtcgt
ttctgaccatccaccagcacagttccggaagcgtcaggatatgcgactcg
ggaaaaccagaatcacgccgaatgacttccagaatccaggataccaggtt
tcctgccgctatacctgcaagctgttcggtatgctgccccgacaaagtgt
gatcgcaatgccagcacaggcgaatacttcctggtgggtgccgcattgtt
gtgaagttcttgtcgtgccacgatgaatgtggccactggcattcaaaccg
attactcaaccattgctcaagggaaggaagcccaccagcacgctgaataa
cccgatcattctcgaagacctgccgcattaccggatcatcagccagcggc
tgaatggcggcgggaacagctcctgtactgaatgacgccatttcttctgg
ttcaggctcaagcagaacgcgaccgcgcataaagaggtgcatcagttccg
cgccgggacgaaacaacacaatccccatacgatgggcgatctcgggggta
agcagagctctcacgcgacctgccccctggcaatgtgttctgcccacagt
ccaccaatccagcgcacgcctttcgccgtgaaacgtgcctggctgaatgc
atgatttgaggttacggatgtgccggttttcacttcaaaacggcccgcat
caatatgctgatgccgtggggtcatcgttccgccaagacgatacatgatg
tcgttctcaaggaggaataaccgcagatcgggctctttggccttaagcag
ttttgccacctggcggaatgacattgacccactggctgtacagtaccgat
caacaaacgctaccttcggcgccgcggcagccagttcgttagtcaactgc
tgtttttgttctgcaaggtcagctgcaagacgtagggcttcagagaatga
ttgaggaatcgtctgctgctgtgcctgctcaagctcctgccagcgatcaa
ccagacgcgcggtaaactccggcgacagctgcgcgacaacgatataactg
tcccgcttccctatcagataaaccgataccgactgattgaggtgattttt
aacttcccccattggggggagttcaataacaccgcgctctgccaggcgtt
caatggaccgtttaacatggtcatgtcttgattccaccagctcagcaata
tcgctgctggacatggttaacgctgttgttgctaactggctcatactttt
ctccatatcaggcggctgcacccgccggttcatatctgctgattgttatc
tctacccgacctttcggcacaacgggtccccattccaccagcatgcgctt
aatctggctgtcgtcttcccagacacccgcatgcgtcagcgcgtcaaaca
gggctttgttgtaattatcgatatcccggcggcgcgcatccggcgggtac
agagtgatttctaccgctgccagttcagtcgatggcttcgggagacgtcg
taattgctcaatgatcgccacgcaggcagcgctctggtatttacggccag
cggcgctaatgaggtgacgaccggccagcggccccttgttaggggcgcgc
cagtaagtgttcacgctcggaggaaaaggcaggatcagtttcacgcggcc
tctccccgcatattgcgaacaagttcagaagcagcagtaatgatttcgct
ggtggcagtccgctccagccagagttgattgatattggctttcagcttgt
gttgcagtgactcatccagcatgtcagcaccatccacctggtcgaataca
attctaacctccagcggccagatacgggactcgggaagcggatccgatac
tggtttagctttctcacgaatgtgcatgcggatctggcgaatattggacc
aactggaaacatccaggcttcccatggctgcaatgaagtcagtgctgttc
atgccatattcaccggatgcttcaagggcaacagtgcgaatacgttccga
catatccaggcgcacagcagcgtcatcgaattcaatcgacaacagccact
catccacaccgaacaaaatactctcacgaataagcagcttcgctttgtcg
atcgttaaaggtgatacctgagtgaattccggtgcttcgacagaatccgc
cgcccaggtatgcccaaacttcgattcactgaatgtgtattcttctttat
cgccaaacgcagctctaacgcatgcccacgcctcgacaccgctgatagca
aaaatatctttctgggtgagtggcaactctgcttctggcttatcagctgc
aggaggtgtggcagttacaggttgagacttgctggcagcaaattgtgcca
aagccataaacgcccgcccttttgcctccagttctgtgcggttgatatag
ctgaaccgctcgccacgccatgacttatcgaatacagctatggcaccggc
aaaaaacgcgctggtgggtttctgtttttcgtcagcaggtacaaaccaca
caggcagatcgaacccaatgcgcccgcgaatgaatacaatgtgatcggca
tcttccggccaccacgtttcactcggcgcggcttttatcaggaatacata
gcgaccgcccttctcgcgctgggctgctgcgtagttcatgatgtgcgtca
tgccggtgatcgcctgcttctcgtggtactgcgaacggctatacggtggg
ttgccatagccagcgccacccagttcagccagacgttcagaccagtcctg
cgtcagcgcgttatcttcggcggtgtaccatgccgggcatttcgcgttgt
cgtcgtcagcaaacaaatccagaactaatggaccaaatagcgcgttgatc
ccccaaaaaagcagatccggtgtccgccactgatcgccaacctctttcaa
ttcgtgagctggtttgctacgcagtgccgccagcgcctggcaatatttat
tggtcatcatgaacggaaccccgaattttctggcagtgagtaatcaacac
tctggaagtttgcgcggctggctgagttagtctcccatttgccgttaacg
cgttcaggccggccagcactggaccatttggtcgcgctttgcaggtaacc
agggaagttttttggaatgaacagagttgccgggcggaggtattgcgcct
gctcgctatcacgccaatcggcatttttgtaatccactaccaagcacagg
tcatcaacagtgaattgttcccgaagacgggcgcgaatattctccagcga
cgtgctgcatacctggtagcgtgagccagtagtctgattcaggtaagaca
aaacctgtctggcctgatcagtaatcacaacctcagggtcgggttgcgcc
gcaaccggacaagagggttttgaagttacttgtggttcttgttttgattt
tactgacggatccccaccagattctgacgggtcaaaaccgccttttttgc
tggatttcgacgcctcaaattttgacgggtcggattttgatgcatcagat
tttgatgggtcagattttgatgcgtcagattctgacaggtgagaaaaagc
agcctctcgcaacttaacgacgttaagctgatatacgttcgatgcgttac
ggttcccattacggcgctgcttacgggaaagccagccatccttctcaagc
tgagcaagggccgtccttaccgtactttctccagcaccgatttgacgtgc
gatcgtgccaatagacggccagctaacaccctcatcactactgaagtcgg
ccagacgcgccatgatggcaacgctggataacttcatgcctgacgaagcg
catgcatcccatacgtaaccggttaatttagtgctcatggtcgtccttta
attctgtaaatttacgctggaattgttcaagagggctgaagcactcatga
tcgtacccttcgcgaaggtatataacgcgctgtgtatctggctcccagcg
gacaactctgacgggaactccgtagtgatctctgaaccgccggttaactt
cagccattcctcgcgccccttctcgttcatctgaacaaatgcttctacca
tcaagtctgctggctggtagttgcctccatcagccgcgttatttatgatt
tccacatagccgaactgggcatctttacccaccagcggcaaacatctgaa
ttgcttagctggtctgaatcggtttacactgttcatgcgttagtttctcc
actgatacgacacgccaaggcgcccggagctgcacactcgcgggcgtcac
cttttctgcctgttgaaacgaatacgtcaatcgcctgatctgaaacacca
accccataaagcgccataaatcccaggaacccgtgaatctggtggcggag
cttcttactgaataattctgaaagcattttgcgctctgatgaatcaatta
ccccatcagccgctgctgccatcttggcattagccagctcaccagatgct
gctgccgctttcatctcaatgctgtacagctcaacgttatccaggctctc
agcagttggaacatccaccagccatttcccttttcggttcgcctggtact
cggccaagtaacaagaaccagacaggtcctccatccgttccagttctgcc
aaggtaaagaaccgactgccacacttctggtacaggtggttgtggaactg
gtcgatagtcatccctaaatcggaagccatacctaagcgaccatgcttgt
gtgccttacacatcaggcggattgctgtatttatgctgtctaccatgttg
atttccctctggtagttaataatcaacttaaagttgactattgttgttag
cggaaggtatgccgtcatttttgttcggataaatatcaggtcgtaattga
tggggagttactacccatccgccccattggcagagttgaataactctttc
agaaggtactcggttctttgcaatccagttcgcaacagattgaactgatt
ggaattcaaaccgccttgatacctctgaaatcgacccgatcgccttcaca
gctttagctgttacattcttgtgttgagatgacatgtgttctcctatgac
taagcctgcatcaatactacttatagtagcaattattagcaacttaaaat
agaaatgacaactatgccttgtgcgcttaatcttctacttatggtggaaa
atgctaaatacaaagactttgccgaaaggctaaacaggtctctccaagag
caatctattggagttaaagaattgtcagagttcagtggtgtctcgtatga
gatggcgcggcgctacactcttggtactgcaaagccgagagatgagaaga
tgattcgaattgcagaaagacttgccgtctcaccggcttatcttgattat
ggtgtgcctgttaatggtggcgacgcgccagccaaaggcacggtcagaat
agagcaattggatgttcatgcttcagccggttccggatatataaaccaac
cattccctacaatagtgagctcaatagagattccagaagagaggatcttc
gagttgtttggtcgtagaagccttgatggcatcgtcatgataaatgttga
tggcgatagcatgatgcccacgctttgcccaaaggacctgcttttcatag
acagcaaggttgaacaattcagcggcgacggcgtttatgtgttcaatttt
gaagacagtacgttcgttaaacgtttgcagaaggtaaaagggcgccgact
ggcagttctttcagacaatgaacattacccgcccttcttcatagaggagc
atgaaatgaatgaactatacatattcggcaagctaatcagatgcttacct
ctaaaaatgatagagtttggctaataattaattcatcaagaaaccggcga
aagccggttttttttacgcctccaattcctcacctcataacactacacta
ctaaaaatttcattttctactttttgttgttgcaattatctacttaaagt
agctatagtcattgcatcgaaagcgaacaggcaggacgcccacgaagtag
ccgccggtggcatatgaataaccggatgattcgctgacagaaaacttagg
ttgggggtagaggtttacatgaatcatttattcacatgctcattttgcgg
agcaaccgaactgggagcgataaagatcgtcgcaaaaggtggtaaggacg
aacctgccatctgttcggaatgcgtagtcacatgtgtagaaaaaatgatc
ctgactaaaaaatcagaggctgaaaaaccaacctctgataacgaaataat
atcagtcgataaaaaactatttaaagagcttcttcagcttgtcctcaacc
ttcctgatttcggaagtaagctggctgctgttgacattgatagtagctcc
acatcgacaagtgaaacttttgttcgacttgagccaagcgattttcttct
tcgtcttagtgccgcacttagggcatgcgggtaacgtaatttcctggtta
tcaaaagcgcccataaacatccctcttggttgtgtgagaacaccaagata
ccaccgcgcctgatgtggttaaaagcaggctaaagcaataacaagtaact
ccctgttctggcggcccggtgttttcccgtgtatttccggtaaccgccag
cctttttcagggcacaacagaaaagggcatcaccgggcgacgggctcata
acccaatccacccgggcaaaaagaaagcggtctctgcaagccgccgacca
atgcaggtgcccttctctgttgtgtatggagaaactaactttttagcgtc
tgtgcagatgcgctgaggaaccgagaatgaataatccgtttttcaaaaat
atgttggtgtatcgcattagtcgcgatttcaccatcaaccaggaagagct
ggaacagcagcttgaactatttcgcttcactccatgcggtagccaggata
tggcaaaaaccggttgggtatcaccacttggtcagctgtcagatcgcttg
catcacactgtcaataatcaagtgttgttggttattcgccgggaagaaaa
aatactgccatctcctgtcattactgaagaactgcgcaagcgtgtgtcgc
gtctagaatccgatcaggggcgtcgcctcaaaaaaactgagaaagattcg
ctgcgtgatgaagtgttgcactccctgcttcctcgggcgttctccaaaaa
ctcgactgttggtttgtggatcaacgtcaccgacggtctgatcatggttg
atgcagccagcgctaaacgtgccgaagactcactggccctgcttcgtaaa
actctcggttctctcccggtggtaccgctgactatggaaacgccgatcga
actaactatgaccgactgggttcgttccggtagtgcgcctgctggctttg
gcctgggtgatgaagccgaactgaaagctattcttgaagatggcggtatt
ggacgctttaaaaaacagactctggtcagtgacgaaattcatgtgcatct
ggaagctggcaaagtagttacaaagctgtctatcgactggcaacagcgca
ttcagttcgttctttgcgatgacggcagcatcaaacgccttaagttctct
aatgagattacagaacaaaacgacgatatcgaccgtgaggatgcggctca
gcggttcgacgctgactttgttctgatgaccggcgagcttatctctctca
ttaacggattaacaacctctctcggcggcgaagccaagcgataaacacca
ggcaacaattacccccataagcatgggttgggttgctgcacgctaaattc
agcaattcattaatttaatggcgcggtgcagcgcgccaatatggagaaaa
ccatgagctacattcagacattatccggcaaacattttaattacctcgat
atccaacaggacgatatcgtgatcgaggatattgctaccgcgttgtctca
tatctgccgctttgcagggcatcttcctgagttttacagtgtcggccagc
atagcgttttaaccagccacctcgttccgcaggagtttgcattagaagca
ctgcttcatgatgctgctgaagcctacctgcaggacatcccctccccact
taagcgcctgcttccggattaccaggcaatcgaagctcgtgtggacgcag
ccattcggcagaagttcggtctaccaactgagcaacacccaaccgtgaaa
tatgccgacctggtgatgctcgccagcgaacgccgcgattttgagattga
cgaaggttccatttggccatgcctcgagggagttgtcccaacggatttat
tcattatcaacccagttcgtcctggccagtcatacggcatgttcatcaat
cgctttaacgagttgatggagcagcgccaatgcgccgcatgaaggtaaaa
gaactcgtagcggaggcgtttgcctccgttgctgaattgccaccaaagca
tgcgccgcttatgcgcgaagtcgccaccagactggacgctacgttcgcag
cattaaaagagtctctggtgcaactggaacaggaacgtaaagataaaacg
ccatgaccgtatttgaatatctccaggctcatccgaataccaccagcggt
gaaatcgccaaaggtatgaacaaaaagaccccagcggtcgccggagcatt
atctcagctctatggcaccggtcggatcgtgaagtctggtgttcgcaagg
gtattccaacataccgcattaacgatatgccgtttggttgcagtaacagc
ctaaccatgatgtttaaccagctcttgagcagagccagacaaggagcagc
ccaatgacagcactcaacaaacaggcgctgcgtgaagaattccagttcat
gcaggacaactatagcgacccggcagaccacgatcggcaggtgatttaca
tcgaggcggaggcgctgctggatgagttggaagccaaagactcaacgata
gcagcacaacaacatgagatccgtatgttgctgaatgcgcttgaggaaaa
accatgcccgaaatgcaacgacacaggaatgactgatagtggcggcacgc
agccatggggcgagccgattgagattgaatgcgactgccgacagcaggat
gccaacaccgcagaacttgtagccgctggcattggcgtgaagggggagtg
agatggataaattaatcaaacctaccgccaaaggtaaatatgacggttca
tgtgattatctttgctcggaagatgcgcgattcatcgttatgcgcggcga
ttatacggaagcggaaataattcaggcttctgtgtctcaagatgtaatcg
actcggatggtgcggctgattttgcaagtagcgcccgctattatcagtgc
tggtacaaagttagcccaataggtggtcaggatggctattcaggctggca
tcatcctcgtgattcgccgtgtcgcggtgcatatttcgcatcagttttgc
aatgggattaaggaggactaacccatgacaactaacaaccacccggcgca
cggtcctgtatcactcgatcgcctgcaccagatacgcgaacacctgctgc
atgatacccaatactcaaacggcgggaacagagcctacattctcgctgat
gtattgaaggtgattgatggggctattgcccgcgagctggtacgccgtga
gcatgcagcgtggtcacaggctactttcggcgatgtcggtccagttggtc
cgctgaagcacctttccaaagaagcgctcgaggctgctgctgaaccaggc
gaccttagcgaatgggctgacatgcaattcctgttatgggatgcgcaacg
tcgtgccggtatcagtgatgagcagattacccaggcaatgataaaaaagc
tggctataaataaggttcgccaatggcctgagccgaaagacggggaacct
cgattgcatatcaaagaacagtcagagcaggagaaaaaataagaatgttt
agcctgattcggcgcggtcaaatctacacggacagtagcaactggcccgt
aattatccatagctgtagtgatcactcggtccgaattaaacgcaatgatg
gcgagctgagaacgattagcatcaaacgctttaacgaagattttgaacga
gtggagcatgatgagtatcgcaaaatatgtgccgaaatagagcaggaaac
aaacctgaaaaacctacgtgcgatgcgtcgcggcaagattactgaatagc
caaacaggagaatatttaacgtgaacaacttaatgatcgaccttgagtcc
atgggcaaaaaaccgaatgcccctattgtctccattggtgccgtattctt
cgatccgcaaagcggtgaactgggtcaggagttttacaccgctgttaatc
ttgaaagcgctatggagcagggagcggtgccggatggtgacactattctg
tggtggttaagacaaagctcagaagcacgatcagcaatctgtgttgatga
tgcgatgccgatatcatctgccctatctgaactgagccatttcattaatc
ggcattctgataaccctaaatatttaaaagtttggggcaatggagctact
ttcgacaacgttatattgcgcggcgcatatgagcgtgccggccaggtttg
cccgtggcaattttggaatgatcacgacgtcagaaccatcgtcacattag
gcagatctgtaggtttcgatcctaagcgtgatatgccatttgatggggtt
gcacataacgcactggctgatgcccgccaccaggcgaaatatgtttcagc
gatttggcagaaactaatcccaaccaccagcaacagctaaagttttcccc
gggtgcagccgggataatggagaaataactatgagcaatattttccagtt
agctcccaacgattgggtttgtgaaagcgttttgatcgcggttactgggc
tcaaacccggaaccatcctccgtgccagaaaagaatgctggatgattggg
agggagtatatccacgtatcgcctgacggaaatcctaaaccttccagtga
gtgcatgtataacagaaaggctgtagatgcctgggtcgcttcaatgaaaa
gcaagcaaccagggtgatttgatgccatgaaaaaggtaagctcgtatcgc
tcttgggcgtctggaggtaacaccaatggataaagtcacatatccaacag
gcgtcgaaaaccacggtggcacattacgcatctggtttaattttaaaggt
aagcgtgtcagggaaagtctcggtgtccctgacaccgctaagaacaggaa
gatagccggggaactgcggacatcagtatgttttgccatccgcacaggaa
cctttgattatgcaacccagtttcctgactcccctaacctcaaggctttt
ggtgtaagtaaaaaagacattacagtgaaagaacttgaagaaaaatggct
ggatctgaaacggatggaaatctgcgcgaacgcatttaatcgctatgaat
ctgtcgcaaggaatatggtgccgaggatcggaggtaatcgcctggtgtca
gcagtaaccaaagaggaattgctgtatctgaggaaatatttgctaactgg
ttatcagaatccgacgaaaaacaaagccccggcaaaagggcgaagcgttg
ttactgtgaactattacatgacgacaatggccggaatgtttcagtttgct
gcggatcacggttacttagaggtgaacccattcgagggaattaagcctct
gaaaaaagccagggcagaaccagatcctctgtctcgtgatgaatttattc
gcctgatagatgcatgccggcatcagcagacgaaaaacctgtggtcatta
gcagtgtacacaggaatgcgtcacggggaactggtctccctggcctggga
agatatcgacctgaaggctggaacaattaccgtcagacgtaattatacga
aacttggtgagttcactctaccgaaaaccgaggcaagcacagatcgagtg
gtgcatcttatccagcccgcaatcagtatcctgaaaaatcaggctgaaat
gacaaggctgggcaggcaatatcacattgaagtgcagttacgtgagtacg
gccgttcggtgaaccatgagtgtacattcgtctttaatccgcatgtggtc
agacgcagtaagcaggtcggatttatctaccgggtcgattcagtaggcga
ctcatgggaagcggcacttaagcgtgcggggatcagacacagaaaggcgt
accagtcacgacacacctatgcgtgctggtcattatcagctggtgcaaac
cctagttttattgccagtcagatggggcatgcgagcgcgcagatggtgtt
caatgtttacggtgcatggatggctgacagcagcgcagagcagatcgcaa
tgctgaatcagaagctggcagattttgccccattgatgccccatagccac
gagaacagtacgggaggattattaaaatcagtaagttaacccctaacgcc
cgtcatgttaactgtgtggagggtaacaccacgctttatgccctgccgaa
acccgaggttgtcctgcgctggcgtgagcagaccacagatgacttccgct
tctgttttaagtttccggcgaccatttcgcatcaggcagcattacggcat
tgcgatgatttagtgactgaatttttgacccgcatgtcaccgttggctcc
gcgcattggacaatactggctgcaactgcctgccacattcggcccacggg
agctgcctgcgctttggcattttctcgattctcttcccggtgaatttaat
tatggggtggaagtccgccatccacagtttttcgccaaaggggaagagga
acaaacgcttaatcgcggtttacatcagcgcggcgttaatcgggtgattt
tagacagccgcccggttcatgcagcacgtccatacagtgaagctattcgc
gacgctcaacgaaaaaaacctaaagttccggtacatgctgtactgacggc
gaaaaatccactgatccgttttatcggtagtgatgatatgacgcaaaacc
gggaattatttcaggtctggttacaaaaattagcgcagtggcatcagacc
actacgccttatctttttttacatacgccagatattgcccaggccccgga
actggtacataccctgtgggaagacttacgtaaaacgcttccagagatcg
gagcagttccggctattccacagcaatcttctcttttctgaatttgccac
ctatcatagacaggtgccatcggccattttaaagggagtttgtatggtaa
gcgcgctgtatgccgttttaagtgcgttgttattaatgaagttctctttt
gatgtcgttcgcctgcgaatgcagtaccgcgttgcctatggcgacggcgg
ttttagcgaactgcaaagcgctattcgcattcatggtaacgcggtggaat
atattcctatcgcgattgtgttgatgctgtttatggaaatgaatggcgca
gaaacctggatggtgcatatttgcggcatcgttttgcttgctggtcgtct
gatgcattattacggttttcatcaccgtctgttccgctggcgacgttctg
gcatgagcgccacctggtgtgcgctgttgctgatggtgctggcgaatctt
tggtatatgccctgggagttggttttctccctgcgttagcgcacaatacg
ccactttctttttcccggatttttacgttatgtctcaccgcgacacgcta
ttttctgcccctatcgccagactgggcgactggacctttgatgaacgggt
agctgaagtcttcccggatatgatccagcgttccgttcccggctattcca
atattatttccatgattggtatgttagccgagcgcttcgttcaacctggt
acgcaggtttacgatctgggttgttctctgggcgcggcgacgctctcggt
gcgtcgcaacattcatcatgataattgcaaaattattgccatcgacaact
ccccggcgatgattgaacgctgccgtcgtcatattgacgcctataaagcc
cctacgccagtagacgttattgaaggtgatattcgcgatatcgccattga
aaacgcatcgatggtggtgctgaattttaccctgcaattcctggaacctt
ccgagcgccaggcgttactggataaaatttatcaagggctgaacccgggc
ggtgcgctggtgctttcggaaaaattcagtttcgaagatgccaaagttgg
tgaactgctgttcaacatgcaccacgactttaaacgtgccaacggttaca
gcgaactggagatcagccagaaacgcagcatgctggaaaacgtgatgctg
accgattccgtggaaacccataaagcacgcctgcataaagccggttttga
gcatagcgagctgtggttccagtgctttaactttggttcactggtggcat
taaaagcagaggacgctgcatgatcgactttggtaacttttattctctga
ttgccaaaaatcatctttcacactggctcgaaacgctgcccgcgcagatt
gctaactggcagcgcgagcagcagcacgggctgtttaagcagtggtccaa
cgcggtggaatttctgcctgaaattaaaccgtatcgtctggatttattgc
atagcgtaaccgccgaaagcgaagagccactgagcgccgggcaaattaag
cgcattgaaacgctgatgcgcaacctgatgccgtggcgcaaagggccgtt
ctcactgtatggcgtcaacatcgataccgaatggcgttccgactggaaat
gggatcgcgttatgccccatctttctgatttaaccgggcgcaccattctt
gatgtcggctgtggcagcggttatcacatgtggcgcatgattggcgcagg
ggcgcatctggcggtgggtatcgatcccacgcagctattcctctgccagt
ttgaagcagtgcgtaaactgctgggtaacgatcagcgcgcacatttgtta
ccgttaggtattgaacaacttccggcactgaaagcctttgataccgtctt
ttcgatgggcgtgctttatcatcgtcgttcaccgctggagcatctctggc
agttaaaagaccaactggtgaatgaaggcgaactggtgctggaaacgctg
gttattgatggcgacgaaaacacggtgctggtgccgggcgatcgttacgc
tcaaatgcgtaatgtctatttcattccttccgcgctggcgctgaaaaact
ggctgaagaagtgtggttttgttgatattcgcattgcagatgtgagcgtt
accaccacagaagagcagcgacgcaccgaatggatggtcaccgagtctct
ggccgattttctcgacccgcatgatccgggtaaaacggtggaaggttatc
ctgcgcctaaacgcgcggtgctgattgcgcgcaagccgtaaaggtctggt
aatactgccggatgcggcgtgaacgccttatccggcctacaaagtcttgc
taattcaatatattgcaggggctatgtaggcctgataagcatagcgcatc
aggca

TABLE F
Sequences of the genes comprised in Phage 3 of E. coli Nissle

| Description | Sequence | SEQ ID NO |
|---|---|---|
| ECOLIN_09965 | ttatttgatgggataaagatctttgcgcttatacggttggatttcgcccggtttgcgagttttca | 135 |
| | gcaattttaatatccaggtgtattgttctggtcgcggaccaacaaaaatctcgacttcttcatt | |
| | catccgccgcgcaatcgtatgatcatccgcctctaacagatcatccatcggtgggcgcac | |
| | ctgaatcgtcagacgatgcgtcttgccatcataaatcggaaatagcggtacaacgcgcgc | |
| | acggcacactttcatcaaacgaccaatcgcgggcaacgtcgctttataggtggcaaagaa | |
| | atcaacaaattcgctgtgttctgggccatgatcctgatcgggtaaataatatccccagtaac | |
| | cctgacgtaccgactggatgaatggtttaataccatcatttctcgcatgcagacgaccacc | |
| | aaagcgacggcgcaccgtgttccagacataatcaaaaaccgggttgccctgattatgga | |
| | acatcgctgccattttctgcccttgcgaggccatcagcatggcaggaatatcgacggccc | |
| | aaccgtgcggcaccagaaaaatcactttctcgttattacgtcgtatctcttcgatgatctcca | |
| | gcccttgccagtcaacgcgcggctgaattttctccggcccgcgtattgccaactcagccat | |
| | cattaccatcgcttgcggcgcggtggcaaacatctcatctacaatcgcttcgcgttcagctt | |
| | cactacgttctggaaagcagagcgacagattgattaacgcacgacggcgtgagctttttc | |
| | ccagtcgtccggcaaaacgtcccagccgtgccagaatgggatcacggaactttggcgg | |
| | cgttaaagcgatacccgccatcgctgctacgcccagccatgctccccagtagcgcgggt | |
| | ggcgaaaggatttatcaaactcaggaatgtattcgctattattttttttcgtttccat | |
| ECOLIN_09970 | ttaatcaaaccgtagctgcggcacaatctctttggcctgtgccaggaattcgcgacgatcg | 136 |
| | gagccggtcagcccttcggtacgcggcagttttgccgtcagcgggtttacggcctgctgg | |
| | tttatccatacttcatagtgcagatgcggcccggttgaacgtccggtattaccggaaagcg | |
| | cgatacggtcgccacgtttcaccttctgtcccggtttcaccaggatcttgcgcaagtgcata | |
| | taacgcgtggtgtagctgcgaccatgacgaatagccacataataacctgctgcgccacta | |
| | cgtttggcaaccaccacttcaccgtcacccactgaaagcactggcgtaccttgtggcatg | |
| | gcaaaatcaacacctctgtgtggcgcaacgcgaccggtcaccggattagtacgacgcg | |
| | ggttaaagttagatgagatacggaactgtttcgccgtcgggaatcgcaagaatcctttcgc | |
| | cagaccagtaccgttacgatcgtagaatttgccatcttcagcgcggattgcgtaataatcttt | |
| | accttctgaacgcaaacgtacgcccagcagctggctttgctcacgtttaccatcaagcattt | |
| | ctcgtgacattaacaccgcaaattcatcgccttttttcagtttgcggaaatccatttgccactg | |
| | catggctttaatcactgcgctcacttcggcgctggttaaaccggcgtttctggcgctggca | |
| | acaaagcttcccccgacggtacctttcagcagattgttgacccactctccttgctgcatttcg | |
| | ctggtcattttaaaaccgttagcggcagtacggtcataggttcgggtttcacgacgagaca | |
| | cttcccaggtgaggcgctgcagttcgccgtccgcggttaatgtccaggagagttgttgac | |
| | cgattttcaggttacgcaattctttgtcggcagcagccagttgggtgatatcacccatatcaa | |
| | taccatactgattgagaatgctgcttagcgtatcgccagtggaaacaacatattcatgcacg | |
| | cccgcttcaccggcgattttgtcatccagttcgtcctggggaatggcttcatcttcttgtgca | |
| | gcttgatcaatcggctcactggcttcaggtaagagcgaacgaatttcgttctgttccagctc | |
| | aatggttttgacaattggcgtggcatcgcggtgataaacatagggccgccagacagcga | |
| | cggccagagtaagaacggtgagcgaccccaacataacgcggtgtggtcgcggtaaatt | |
| | attaaacgccagggcgacagagcgggctatctgttgcac | |
| ECOLIN_09975 | ttaatctcctttcaggcagctcgcatactggttggctaattgattcaggaattctgaatagctt | 137 |
| | gttttacccagtttgatattcgtccccaggggatccaacgttcccatacgaacggatgtccc | |
| | tcgtgcgacgctctcaacgaccgctggcctgaactgtggctcagcaaaaacgcaggttg | |
| | ctttttgctcaaccaactgtgttcttatttcatgtaaacgctgcgcgccaggttgaatctcagg | |
| | gttaacggtaaaatgaccaagcggtgtcagtccgaactgtttttcgaaatagccgtaagca | |
| | tcgtgaaaaacgaaataacctttccccttgagcggcgcgagctcgttaccaacctgcttttc | |
| | ggttgaggctaattgtgcctcaaaatccttcaggttggcgtcaagtttggctcgactttgcg | |
| | gcataagttccactaattttccatggattgcaaccgctgtagcccgcgctatctctggggaa | |
| | agccaaagatgcatgttgaaatcgccgtgatggtgatcttcgtcacttttttccgcgtggtcg | |
| | tgatcatcatcatcgccgtgaatacttttcatcagcagcggtttcacattctctagctgcgca | |
| | atcgttacctgtttcgcttcaggtaatttacttaccggtttttgcatgaacgcttccatctccgg | |
| | gccaacccaaacgactaagtccgcgttctgtaagcgttttacatctgatggacgcagtgaa | |
| | taatcatgttctgaagccccgtcaggtagtaaaacctccgtttctgttaccccatcagcaatg | |
| | gcagaagcgatgaacccaacgggtttaagcgaagcgacaacggcagcatctgcggcct | |
| | gtgttgcaccgccccagagagcggcggataatgctgcgaaaagaagcgtttttttatgtaa | |
| | cat | |
| ECOLIN_09980 | atgacaagtctggtttccctggaaaatgtctcggtttcttttggccaacgccgcgtcctctct | 138 |
| | gatgtgtcgctggaacttaaacctggaaaaattttgactttacttgggccaaacggcgcag | |
| | gtaagtcgacactggtacgggtagtgctcgggctggtaacacccgatgaaggggttatc | |
| | aagcgcaacggaaaactgcgcatcggctatgtaccgcagaagctgtatctcgacaccac | |
| | gttgccactgaccgtaaaccgttttttacgcttacgccctggcacacataaagaagatatttt | |
| | gcctgcactgaaacgtgtccaggccgggcatctgattaacgcaccgatgcaaaagctct | |
| | cgggtggcgaaacgcagcgtgtactgttagcgcgagcattgttaaatcgaccgcaattatt | |
| | agtgctggatgaacccactcagggcgtggatgtgaatggtcaggtggcgttatatgacctt | |
| | attgaccaactgcgtcgcgaactggattgtggcgttttaatggtatctcacgatctgcatctg | |
| | gtaatggcaaaaaccgatgaagtgctttgcctgaatcaccacatttgttgttccggcacacc | |
| | ggaagttgtttccctgcatccggagtttatttctatgtttggtcctcgtggtgctgaacaactg | |
| | ggtatctatcgccatcatcataatcatcgtcacgatttacagggacgaattgttttgcgtcgg | |
| | ggaaatgatcgctcatga | |
| ECOLIN_09985 | atgattgaattattatttcccggttggttagccgggatcatgctcgcctgtgccgcgggtcc | 139 |
| | gctgggttcgtttgtagtctggcgtcgtatgtcttatttcggtgatacgctggctcatgcctca | |
| | ttacttggcgtcgcgtttggtttgttgctggacgtgaatccattctatgcggtgattgccgtta | |
| | cgctgctgctggcgggcggtctggtatggctggagaagcgtccacagctggcgatcga | |
| | cacgttattagggattatggcgcacagtgccctgtcgctgggcctggtggtcgttagtctg | |
| | atgtctaatattcgtgttgatttgatggcttacctgttcggtgatttactggcagtgacgccag | |
| | aagatctcatctctattgcgattggcgtggtcatcgtggtggctattttgttctggcaatggc | |
| | gcaatttgctgtcgatgacgattagcccggatctggcgtttgttgatggtgtgaaattacag | |
| | cgcgtgaaattgttgttgatgctggtgacggcattgacgattggtgtagcgatgaaattcgt | |
| | cggcgcgttgattattacttcactgctgattattcctgctgctactgcacgtcgctttgcccgc | |
| | acgccggaacagatggctggtgtcgctgttttggtggggatggtggcagtgactggcgg | |
| | tttaaccttttccgcattttacgatacacctgcaggcccgtcggtggtgctatgcgcggcac | |
| | tgttatttattatcagtatgatgaaaaagcaggccagctaa | |
| ECOLIN_09990 | ttacggcatttctggcggcgtgatgccgaagtggttccacgcccgcactgtcgccatacg | 140 |
| | cccgcgcggtgtacgctgcaaaaagccttgctgaatcaaataaggttccagtacatcctc | |
| | aatggtttcacgttcttcgccaatggctgccgccaggttatccagacctaccggcccacca | |
| | aagaacttatcgattaccgccagcaacaatttgcggtccatataatcgaaaccttcagcatc | |
| | gacattcaacatatccagcgcctgagcagcgatatctgccgagatggtgccatcgtgcttc | |
| | acttcagcgaaatcacgcactcgacgcagcagacggttggcaatacgtggcgtaccgc | |
| | gcgcacgacgagcaacttccagcgcgccgtcatcactcatctcaagccccataaagcgt | |
| | gcgctgcgactgacgatatattgcagatccggcacctgataaaactccagacgttgcaca | |
| | ataccaaaacgatcgcgcaacggtgatgtcagcgaacctgcgcgcgtggttgcaccaat | |
| | cagggtaaacggcggcaaatcaattttaatggagcgtgccgccggaccttcaccaatcat | |
| | gatatccagttggtaatcttccattgccggatacaacacctcttccaccactggtgaaagac | |
| | ggtggatctcatcaataaacagtacatcgtgtggttcaaggttagtgagcattgctgccag | |
| | atcgcccgccttttccagcaccggaccagaagtcgtgcgtaaattaacgcccatttcattg | |
| | gcgacaatattggcaagcgtagttttacccaaccccggaggaccaaaaatcaatagatga | |
| | tcgagggcatcgccgcgcagtttcgctgctttgatgaaaatctccatctgcgaacgaacct | |
| | gcggctgaccaacatactcttccagtaatttagggcgaatggcgcgatctgccacatcttc | |
| | cggcaaagtggtaccggcagaaatcagacggtctgcttcaatcat | |
| ECOLIN_09995 | tcataacgcggcgcgtagggcttcgcgaattaatgtttcactgctggcgtcagggcgagc | 141 |
| | gattttgctcaccatgcggcttgcttcttgtggtttatagcccagtgccaccagcgcagcaa | |
| | ccgcttcctgttcagcatcgtcggtcgccgggctggcaggagacgtgagtaccaggtcg | |
| | gcggctggcgtaaagagatcgccatgcaaacctttaaatcggtctttcatttcgacaatcaa | |
| | gcgttcggcggtttttttgccaatacccggcagtttcaccagtgcccccacttcttcacgctc | |
| | aacggcattaacgaactgctgcgctgacattccggagaggatcgccagcgccaacttcg | |
| | ggccgacgccgttggttttgatcaactctttgaacaacgtgcgctcttgtttattgttaaaacc | |
| | gtacagcagttgcgcgtcttcacgcaccacaaagtgggtgaaaacgatcgcttcctgacc | |
| | cgcttcagggagttcataaaaacaggtcatcggcatatgcacttcatagcctacgccgcc | |
| | cacttcaattaacaccagcgggggttgtttttcaatgatgatgcctctgagtctgcctatcac | |
| ECOLIN_10000 | gtgaatattaattatcctgctgaatatgaaattggtgatatcgtctttacatgtataagtgctgc | 142 |
| | cttatttggtcaaatatcagctgcatcaaattgctggagtaatcacgtcgggatcattatcgg | |
| | tcataacggtgaagactttctggttgcagaaagccgtgttcccctctcaaccatcactacgc | |
| | tatcccgttttattaaacgctctgctaatcaacgctatgctataaagcgattagacgccggac | |
| | taacagaacaacaaaatcaacgaattgttgaacaggttccttcccggctacgcaaaattta | |
| | ccacaccggttttaaatacgaatcttcgcgccagttctgttcaaaatttgtttttgatatttataa | |
| | agaggcgctatgtattccggtgggtgaaatagagacgtttggagaattgttaaatagcaat | |
| | ccaaatgcaaaactcactttctggaaattctggttcttaggttctattccgtgggagcgtaaa | |
| | accgtcacgccagccagtttgtggcatcatccgggtttggtgttgattcacgcggtgggag | |
| | ttgaaacgcctcagcctgaactgaccgaggcggtataa | |
| ECOLIN_10005 | ttaacgcagtcgccctctcgccaggttcagtcgcgattcgctcatttgcatcgcattctgact | 143 |
| | aacgtggcagtgggtgatggcaatcgccagcgcatcggcggcatccgcctgtggattag | |
| | cgggcagtttcagcaaggtgcggaccatatgctgcacctggcttttttcggcactaccaat | |
| | acctaccactgtttgctttacctgacgtgccgcatattcaaataccggcaattcctgattcac | |
| | cgccgccacaatcgccacgccgcgcgcctgccccagtttcagggctgagtcagcgttct | |
| | tcgccataaagacctgttcaatggcgaaataatcaggctggaattgggtgatgatttccgtc | |
| | acgcccgcatagatgagcttcagacgagacggtaaatcatccactttggtgcgtatgcatc | |
| | cgctacccaggtaggacagttgcctgcctacctggcggatgacgccatagccggtcacg | |
| | cgcgaacccgggtcaatgccgagaataatagccat | |
| ECOLIN_10010 | tcagagagtcgctgcaacctcatcagagatttcaccgttatggtaaacttcctgcacgtcgt | 144 |
| | cgcaatcttccagcatatcgatcagacgcatcagtttcggtgcggtttctgcatccatatca | |
| | gctttggtggacgggatcatggaaacttccgcgctgtctgctttcagacctgccgcttcca | |
| | gagcgtcgcgtactttgcccatttcttcccatgcagtgtagacatcaatcgcgccgtcatca | |
| | taggtcacaacgtcttcagcaccggcttccagggctgcttccatgatggtgtcttcatcgcc | |
| | tttctcgaaggagatcacgccttttttgctgaacaaataagctacggaaccatcagtaccga | |
| | ggttaccgccacatttgctgaatgcatgacgcacttcagcaacggtacggttgcggttgtc | |
| | agacagacattcaatcatgattgccgtgccgccaggaccgtaaccttcgtagatgatggtt | |
| | tccatgtttgcatcatcatcaccgcccacacctcgtgcaattgcgcggttcagagtgtcacg | |
| | ggtcatgttgttagacagtgctttatcaattgctgcacgcaaacgcgggttagcgtccggat | |
| | caccaccgcccagcttagccgcggttaccagctcacgaatgattttagtgaagattttacc | |
| | gcgcttagcatcctgcgcagattacgatgtctggtgaggcccatttactatgacctgccat | |
| nudB | tcaggcagcgttaattacaaactgttcaatcgcctgccggttgctccaggacttagtgagc | 145 |
| | gccgccgcagcagacgcatcaagccacttgtaagccagatgttcagtgaaaacgatctg | |
| | gcgctcgtgcggaagcgcaagacagaaccatgattccgtattacgcgtcacgcccggc | |
| | gcatagcgatgacgtaaatgtgaaaaaatttcaaactctaccgtgcgctgacagtcaatta | |
| | aggtcagttgttcagcgacaacatcaatggtgacctcttcctttacttcgcgcatggcagctt | |
| | gcggcgcggtttcaccctcttccacgctgccggttaccgactgccagaaatcgggatcgt | |
| | cacgccgctgcaacatcagcacccgtttcgtatcttgtgcgtagatgaccactaagatcga | |
| | aacgggacgcttataagccat | |
| ECOLIN_10020 | tcagttattctcagccttcttcacaacctgaatgctcagctcagccagtgcagtcgggttag | 146 |
| | caaagctcggcgcttcagtcatcaaacacgctgccgccgtggttttcgggaaggcgataa | |
| | cgtcacggatattgtcggtgccggtcagcagcatcgtcagacggtcaagaccgaatgcc | |
| | aaacctgcgtgcggcggagtaccgtatttcagggcgtcgagcaggaagccgaatttctc | |
| | gcgctgacctatcgttgatacccagaataccaaacaccgtctgctgcatatcaccattatg | |
| | gatacgcacagaaccaccgcccacttcgtaaccattgatgaccatatcgtaagcgttagc | |
| | caccgcattttccggtgcagctttcagttctgctgccgtcatgtctttcggtgaggtgaacg | |
| | gatggtgcattgctgtcaggccgccttcaccgtcgtcttcaaacatcgggaagtcgataac | |
| | ccacagcggtgcccatttgctttcgtcggtcagaccaaggtctttacccactttcaggcgc | |
| | agtgcgcccatcgcgtcggcaacaattttcttgttgtcggcaccgaagaaaatcatatcgc | |
| | catcttgcgcgccagtacgctccaggatggcttcgatgatttctgcattaaggaacttcgct | |
| | accgggctattgataccttccagacctttcgcgcgttcgttaactttgatgtaagccagacct | |
| | ttcgcgccgtagattttaacgaagttaccgtattcgtcgatctgcttacgggtcaacgatgc | |
| | gccgcccggaacacgcagagcggcaacacggcctttcggatcgttcgccggacctgca | |
| | aatactgcaaactcaacagatttcagcagatcggcaacgtcggtcagttccatcgggttac | |
| | gcagatccggtttatcagaaccataacggcgttctgcttctgcaaaggtcattaccgggaa | |
| | atcgcccagatccacgcccttcacttccagccacagatgacgcaccagcgcttccatcac | |
| | ttcacgcacttgcggcgcggtcatgaaagaagtttccacatcgatctgagtaaattcaggc | |
| | tgacggtcagcacgcaggtcttcgtcacggaagcatttaacgatctgatagtagcggtca | |
| | aagccggacatcatcagtagctgtttgaacaactgcggggattgcggcagcgcgtagaa | |
| | tttacctttgtgcacacgagaaggcaccaggtagtcacgcgcgccttcaggcgtggctttg | |
| | gtcagcatcggagtttcgatgtcgaggaagccgtggtcatccataaaacggcgcaccag | |
| | gctggtgattttagcgcgggttttcaggcgctgagccatttccgggcgacgcaggtcgag | |
| | gtagcggtatttcagacgcgcttcttcggtgttgacgtggttagagtcaagcggcagaaca | |
| | tctgcacggttgatgatagtcagcgaggacgccagtacttcgatttcgccagtcgccatat | |
| | cgcggttaatatttttttcgtcacgcgcacgtacggtgcccgtgacctgaatgcagaactca | |
| | ttacgcagttcagaggccagctttaacgcgtccgcacgatccggatcgaaaaatacctgc | |
| | acgataccttcgcggtcgcgcatatcgatgaagatcaggctaccaagatcacgacgacg | |
| | gttgacccaaccacacagagtcacctgctgccccacgtgggacaaacggagctgtcca | |
| | caatattctgtacgcat | |
| ECOLIN_10025 | atgcttgaacttaatgctaaaaccaccgcgctggtggtgattgatttacaagaaggcatctt | 147 |
| | gccttttgccggaggtccacatactgccgatgaggtggttaatcgcgccgggaagctgg | |
| | cggcgaaatttcgcgccagcggtcagcccgtgtttctggtgcgcgttggctggtctgccg | |
| | attacgccgaagcattaaaacagccggttgatgccccctcccccgcaaaagtgttgcccg | |
| | aaaactggtggcaacatcctgctgcattaggtgcaaccgacagcgatatcgaaatcatca | |
| | aacgtcaatggggtgcgttttacggtacggatctggagttgcaattacgccgccggggtat | |
| | cgatacaatagtgttatgtgggatctcgaccaatatcggtgttgaatccaccgcccgcaat | |
| | gcctgggaactcggttttaatctggtgattgccgaagatgcctgtagcgccgctagcgcc | |
| | gagcagcacaataacagcattaatcatatctacccgcgcatcgcccgtgtgcgtagcgtt | |
| | gaagagatcctcaacgcgttatga | |
| ECOLIN_10030 | tcacatcaccgggcagtcatcaaactccgcattcctggcatcattaatgatgtacgtgatca | 148 |
| | ctccaaatatagcgggtgcagaactgtaaccatcatcatctgctggcagcgcttcccttctc | |
| | ccgttatccagattaaccaggtgcggctgaggatgagtccgatatcgcttgatcctgaattc | |
| | cccgtcgattgcacatatcagcagtgaaccatcgcaggcagtaagtgacgcatccacaa | |
| | caagcaacgctccctggattatcccttccctgaaatgtgaacgcgatgcccgcatgaaata | |
| | agtcgctgcgggctgactgattagctgctgatcgagggagattcgtgtttcaacataatctg | |
| | ccgcaggtgaaggaaatcccat | |
| ECOLIN_10035 | atgttcgtggaactcgtttatgacaaaaggaattttgatggtctgcccggtgcaaaagatat | 149 |
| | cattctgggcgagttaactaagagagttcaccggatcttccccgatgctgatgttcgggtta | |
| | aaccgatgatgacactgccggcgatcaacactgacgccagcaagcatgagaaggaaca | |
| | gataagccgtactgttcaggaaatgtttgaagaggctgaattctggttagtgagtgagtaa | |
| ECOLIN_10040 | atgctgtggaggatattcattttcgtaaacgttggtttgggagaagcggcaaaacggaatg | 150 |
| | tgggaacaggggaaaatcagataccagatatgtctgcatttccatctggcaataactggttt | |
| | cagttaccaagtggacatatcgttcagatattttccatgaacgttcttggtgcagatgctaat | |
| | ggcacgtcagctaattaccccattgcttttccaacaacgatgattgctgtcagtgctctatgg | |
| | tctgatgggactgtagcaaatgcaccgacatacaagatgatggggaacacgactaacag | |
| | aacaactttgacgataaaagtatcagccagctcaggtacttacgggacaatgattattgcg | |
| | gtgggacgataa | |
| ECOLIN_10045 | atgaataaatacagttactctccttcagaaaatgccttttatgctgttgcgttaaaaaatacct | 151 |
| | atgaattgagtggcacatggccagctgatgcattagatattcctgatgacatttctgtaaaat | |
| | atatggcggaaccgccacaagggaaaatccgagttgcaggggaaaatggttttcccaca | |
| | tgggctgaaatacctccaccatcacatgaggaacttattgaacaggccgaatcagagag | |
| | gcaattattgattaaccaggccaacgaatacatgaacagtaaacaatggcccggtaaagc | |
| | cgctattggtcgtctgaaaggcgaggaactggcacaatataattcgtggctggattatctg | |
| | gacgcactggaactggtcgatacttccggtacgcccgatattgaatggcctacgcctccg | |
| | gcagttcaggccagatga | |
| ECOLIN_10050 | ctacgcctccggcagttcaggccagatgacatccggcgcggtgctggtatctgttgcagt | 152 |
| | caccgcgtcaatgtaatccagcacggcgttaagtcgggttgtttctgcctgagtcagtttcc | |
| | gtccggcctgtaatttcagctgaatcagactaatggaagccattgctgcatcaatcagtgat | |
| | tggcgctgtgcttctgccgcttctactgaggcaccgtgttgtgcctcagtatctgtcacccat | |
| | ttctcaccatcccatttatcatatggcgttaacggtgaaagcgtgacataaccgtttttgatg | |
| | gcaccgatataatccactgtaacagctgcgccattttcgattgagtaaacagtctcattgcg | |
| | atggtcttcctcatggctccatcccttacctgtaaatactgccactcttcccggaatgttttcgt | |
| | ccgggtcaataccagtggaacaggcgggcatacttacgccagtattaatatattcatcaga | |
| | ccagcccgtatattcagacgttactgcatcataataaaaacaacgcatatcacccggcact | |
| | gcagccagcccattttcatcaaaaacaggtttcat | |
| ECOLIN_10055 | ttatttagccctcaccagaaagttaaatgcaatatttcgcggtctgacagcaacaaaattca | 153 |
| | caccatcacccacagagttactgttgaaattaaatcgtgaaaatcctggctgatttccggcg | |
| | atgccatcatgaaagttaattgcgtgtccagcacctccgcctatattcccggcaaactgag | |
| | aaaagtttgtagcttcctgccagcttaataattcgcgaccaccatctgcacctcgcccgtca | |
| | tcccagacacgaatgaaatcaccgcgggcttcaggtaataccagcgaaggaaacacttt | |
| | cgccagcacaggataatcagtggcagagaatttcgcgccgttgaacttcaaaaacaccat | |
| | actggaccagctgtcgattacagtatttggcattgcagcggacggccagaagaacggaa | |
| | cgccaatagctggagaaccttctcccaaaccaaggtttgtgcgagcgtctgcggcattcg | |
| | ttgcgccggttccgccgtctgcgacagtaaccgcaccgttgctccctttctgcgcaagttta | |
| | ccgatgcctgggatggttacggcggtgccgttgatggtaactgtgatgctttggtttgctga | |
| | ggtggtggcgaacgtctcccacgcgccaatattctcgtcgtactctttgatgagctgtgac | |
| | atggcctgcgccaggccgtcgactgagatattgtccgacacaaggattccatacttctgg | |
| | ccgctcagcgccggggaaactgctggcgtaaccgtcattgacgtggcgctgttcacgga | |
| | tgaaatctgaaacagctgcaccgggttagacatcacgataatcgtctggccagcgcgga | |
| | cctggctggcgggagctgtccagtttgtgccggagccggttgcggtatttccgttaataga | |
| | gatagttccggtgctataaatcat | |
| ECOLIN_10065 | ttaacctgtgaatgaaccagatccgtgtgatactatgatagtaggcgaagaaattgacgca | 154 |
| | cccctgttattttgagcggacactttaatacgcactgttacgtttggacctgttacaaccgca | |
| | gaatgcatgataagtctctccatgtcttgcacggatgaaccactctcattgccgttaatgtcg | |
| | attgttcctgcaccattacctttaacgtttgctataacacagacgtgtcttgcgtgcccagaa | |
| | ctggatgaatcattataagtcattaccttttctaaatatccgtcactagaccgactcacattcg | |
| | tccctgtgtgcatatttgcaatgtccccgataaaactttgggcttccacagtccctttgaattt | |
| | accgcttgttgcttggatctcaccagtaaagctaccgccactagcatatactacacctctga | |
| | cggtcacattgttgaattcagcatctccagctttattcaacttccaaccagcagaaccagct | |
| | gcatagttgttggactggatatagttaccgatttttgcgttctcaatggtgccgtcctggatga | |
| | agctggcccggatgaatgtctgcccgttctggatcacgaacggcaaagctacgctatttc | |
| | cggctgccgtggtgacggcgaagcggtcagccaggaagataacctgcgactgcatgcc | |
| | ggatggcgtattctccacgccgatacccatccccgcggcgtaatactgcccgttgctgga | |
| | gacaccaaccttgatgttgtacatcgcgctgagttcgccattaacgttggctatagcctgag | |
| | cgttagtggtgatggcggaggtatgcccgttcacggtcgccgtgatgccgtttatctgcgt | |
| | ggcggtggcctgctgatagtcggagagcgtctgattcaggctgttgatggatgccttgttg | |
| | ccgttgacgtccgtctgcaggctcagcaatgaacgtgctgtggcttccttctcactgacgat | |
| | cacctcgtcgagacggtccagattcgcgctgttgccggcgaccgatgcagaaagggtttt | |
| | acgcgtggccacctgagcgaggttggcctggattatcgcaattgcagagttcttcacccc | |
| | gcccgtcatgccgtccatagaaacgctgatgttgtcgattcgctggcccagggcggtatc | |
| | agccgtcgcaacggtctgctcaagctgactgagtgaagacgaaacattcccgaccgtgc | |
| | tggaaagctcattaacgctggtctgaaccttcccgacgtcctgggcatttttggcgatatctt | |
| | tcgcttgctgctccagttcgtcgttggcctgtttgatatcgttagccatgccagcaattttttcg | |
| | ttgctgtccaccgcgttctcgatcaggtctttgaacgtttccgactctttcatatcctccagaa | |
| | tgtcattagttatttcgctgacatctatcgaggacgtgcccatgatccagtcggtccagtccc | |
| | cggcgttaccgatacggtcaatcaggcgcgcgcggtaccactggcgaacgccggcag | |
| | gcatggggccatgctgataatctgcagccgggtacggcaccaggaccagcagttcagg | |
| | attggcgtagtcggcagttgtggcgcgctgaatctctgtataggccgtgtcgcctgagcca | |
| | tccggaaatttccaggtcaggtcgatatgccagaccacatcttcggtcgccaggaagttg | |
| | agcggagtacccggttttcccgttttaccggagagataagttgtttcaccgtatccccatgg | |
| | tgacgacgtatcctgcgcattcagcgcccgtacgcgcacgtcatagctgcccgaataaat | |
| | gccctgaaccgagaaaccctgcgcgctggtaaccggaacgtttatccagtccccgttgtc | |
| | cttacgccactgggcaacataccggattgcgccctctaccttatcccatgacacgtccagg | |
| | cttgctacagtcagcccctgagacacatgatcgctctcagtcaccacgatattcttcggag | |
| | cagacaggacgcttatcggcgtgacggtgatcgggggagactcgacccgaacgccgtc | |
| | atcgatgtaacgatatttgtttggatcgtgctgaacggccgtaatagtgaaaccgcctgtac | |
| | tgtcgtcgttagccgcgattgaggtgaccctgaagtactgtattgcgaggttatcactgtct | |
| | atcgcccaaacagcgcccgccacaggaacctgactgaatgccgtagccaccgtcaccg | |
| | tttttttatcggcgctcaccgcgctgattgtccgcgtctgggcttttccgtcgggaaggttaa | |
| | ccaccagccggtctttcgccgcgtagtctatttctcgatcgagggtaatttggcggccgttg | |
| | gccgcgcttatacggcccccgttctccttaccagagcggaaaggatcggcgacaccgat | |
| | aatttcagcgggcaaagggatataaccgtccagccccacgccaaacgatacggtcccgt | |
| | ctttggcattggagagcaatacccagcgaccgcgtcggtgcgcttcactttgcgaggtgc | |
| | agccgattgcggtcagggacgtctgccggacgtcgtaacgttctacaagcgccgaatcg | |
| | taaaccccctcaacggtatcgctgtaatggttctgcggatcggaccaggacaccaggca | |
| | ggagctgtagcgattcttgtatgagccgcccgcataagtaaacagcccatcgataacgttt | |
| | gagacgttataaacccagtcaacatcgtcctgcgggacgtctgcctggacataaatctgat | |
| | cgtttccccagaacgttattccacgaaataccgcggcgagatcgttaagtacctgccagg | |
| | cgtcctcctggctctgaatgaaaacgttgcaggtgaaacgcggttcggtgccaccggcc | |
| | ccgtcggaaaccatttcgtcacagtactgggcgattgaatacagcgcccacttatccacca | |
| | tggacgcatccacgcgcgtgcccatgccgtaaatttcatccagaaccagatcgtaaaaga | |
| | tccaggcagggttattggaccatgccattttgaacccgccggaccatgaaccagaatagg | |
| | ttcgggttttcggatcgtaattatccggaaccttaatcagcttgccttttatcttacaggtcact | |
| | ttcggcgcgctgccgttgaattggctgctgtccacttcgacatacaggagcgctgttaaag | |
| | gataacgaagcttgctgtcgatgacttccgcatacgaaaacaccttgaaggcgttaacca | |
| | gtttcgaatttgatccgctggcatcagccgtaatacgcctgaccctgacagaccagccgg | |
| | acgtggattttggcagatcgatacggtggtcacgctgatattccgtcgtggtctttccgtca | |
| | aacttgccgtttacaaccgttttccaggcgccgccgtccgttgataaatcgatcgcatactc | |
| | ggtgaccgtgcccaccatatcgccattatctttatagagatactggaccggaaggctgag | |
| | cttgatacggatggcatccagggaaaggtttgtaaactggcgcgtccagggcgcggtgg | |
| | tggtgacagttgtgcccacggccagctcgttgtcgacctggggcatcccggcaatatagg | |
| | tctggtcctgtgtgcccttgcggaactcccatttcacgccgctgaagttgtattccccgctgt | |
| | cgtttgccagcggcgtatcgttgagaaaaatgttctgagcggtcaggtcgccctgtatttcc | |
| | ccctcagaaacggcaatgagcatttttaattttgcgaccgacagcagatcgtcaggctgct | |
| | caaccggagtatgtgaactgccacctccccctttggcaccctgcaggatggtttcttgttta | |
| | agaagctgcattttttcacccat | |
| ECOLIN_10070 | ctactgctgatcgctcgagtacataccggcgctgactatcgctccccctgcctcagtcaga | 155 |
| | ccgtaggccagggggacaggatgccccatagcgacggtattgaccggcgccccgaag | |
| | gcgtagttaggcgtgttgtccgtgctggaggatttacccgcgccgaaggatggctgggg | |
| | cgtgagcatctggacaacgccccccagcatcatcgacactccgacccctgtcagaattg | |
| | acgtggcgctgatagctgttgcactcatcgccgcgccccaggctgccatgctcgcacca | |
| | gcggtaaagaatgcagcgaccagcgcaacagccccgacaactatctgcaggacgccc | |
| | gaacttttggccccctcataaacgggcacgatccggtacacgcttccaccgcgggtcata | |
| | tcaaactcttccagcccgatattgttgccaccgttaaaaaaggcgaaacggatccccttcat | |
| | atgagcttcagacatatattttttgaatccgggaacctgtgaacacatggccctgagcatct | |
| | cgcgcaggtcggcaacatcaaactgaacgcgtttaccgaatttttttgccattttaccttcga | |
| | gaataagcgtcttaaccat | |
| ECOLIN_10075 | ttaaccatgcattctgtccttatgcctgaccacccggaccgttctgtcgcgataatattttcca | 156 |
| | taaggcgttcgcgaagaaaggtgcccgaaaagatgatggagaatgatgttatcacccac | |
| | atataccgcggcgtgattagtcaccgatgcctgcacactcatcatgatgatatctccgggc | |
| | tgcattgcaccggcggcaatctcaacgaatccctcacgctcccagttgtcgtcgtagaga | |
| | cgctccttgccgctctcccaccattcgtaaggtactgaataattgccgagaacaatgccgt | |
| | attcgcgcagataaaattcacggataagcgaccagcagtcggcgtaacccagcacccac | |
| | tgccgcccggcataatcccggtcttcacgcggggaaatcgtacaaaaatccccgtccgg | |
| | ccaggacatgatcccccactcaatccccgaccagtcgcactggatccggtccagctctg | |
| | agggcaccagccgaaccacatccggatgggaatgaatgagcatgatgatctcaccgcg | |
| | cgcgcgggcagcgagctggtcttccggggagagcgtgaatgtctcctcgggtttatcgg | |
| | caatgttgcggcagggaataaagatttgttgctggcctgactgaacaatcaggccgcagg | |
| | cttctttggggtattcagcagcgacgtgctgacggatagcatccagcaatttttcacgcat | |
| ECOLIN_10080 | ttatttcccctgcaggtttgcagccggaaaaccgccgaacggcagcggcgcatccgggc | 157 |
| | cgtgacgatcctgacaatcctgccggcggccgccacaaacatctttcgacgggtcatcg | |
| | gtcggtgtaccgtctttggtaaagtatttcgtgccgttgtaatcgcatccggtcccgcttcgg | |
| | taccagccccgcatacaccaggtgcagacaggcgtaatctgccgtgtcggcagctgca | |
| | ggctctgaatatcgaaaggagaacacagctcgaaatcaacctgtacccgcgtctctgcg | |
| | gttttagcattgacgtaaaagagctgtaagcgctcatcggccgggctggcacccggatta | |
| | ccgtttttccagttggcggcatcgagatacttcgaaagcgtggtatggattttgaccttagcc | |
| | ctgaccatatcgtcatattcaagacacagcgcggtgacatagtttccgacgttcccgacgg | |
| | acagcgtgggcgttggctgggaacctgtactcgataactccatccccttaagttcgtaggg | |
| | atggggatcgtactggtttccctgccagataatggcgggcagattttctgcggcgaaggct | |
| | gcccacccctcttcctgaatattgtgcgcatgaaaacgcagcacctgatccataccgaatt | |
| | cagtgccgtcgatctcaatcagctgaataacgctgccgggctcaagctgttgtatgtctgc | |
| | cgtaaaactcat | |
| ECOLIN_10085 | tcagggcgcgaacgcctgttcaaaagtgaaggccacagtggcttttttcccggtagggaa | 158 |
| | agaaacgctgaacgaatcggccttcattctgaacagctttttttcaccccatggagtggtcc | |
| | accagaacgatttagtaacgtgagacatcaggaaagcgcgcagcgcagccgcctcctgt | |
| | ctggtgcccgtccagtccaggttccacgtttcctgtttgtcgttgatccccatccccgctatc | |
| | tgtttgtagccatccccgaactgggcctgcagcgttcgggctgtttcagtgccctgcgctgt | |
| | ttttcgcgtgcgccaggtaaacgtgtccgtcac | |
| ECOLIN_10090 | tcactgtgtcctcctcgaataaagcacgccgcccgcggacatttcttttttcagtcgctcggt | 159 |
| | gattgtctgctgaacaatcgcctgcagctgtttcgccgtccccgtggcgttcgcctgatttat | |
| | gcttccgtcactcccctgctggctgatgctgactggggcataaacactgatcccgcccatg | |
| | ccagcaccggctgcgttcccgccgccgaccagaccacccgaggcatacccgcgcatc | |
| | aggcgatagagattagccacgccgatgcggctggttgattctttggtgaagacgaattcc | |
| | ccgcggtgaacgataccggctggctcgtacttgccgccgtgcccggtaaaaccgccca | |
| | cgtcaaaaccctgtggccggtatgacgggaccgcgaatgactgaccggcagaggaggt | |
| | tttcgccccgccgctaacccagcccattgcactctggatggtgtaagccaccagcagctg | |
| | gttgataacggacacaatcattttaaggatcgagctggtgaattccctgaagctcgccttcc | |
| | cggttgtcgtcaggctggtaagctggcccgccaacccgctgaacgtagcctgagaaatc | |
| | tgctgaacggagctgaaaacgtttgtcgctgaatcctgatattcggcccaaccctgtttcgc | |
| | accggccagccagtttgcacgcagggcatcttcagcttcgaacgtcgccctttgctcttcc | |
| | agaaccttttgctgcgcctgagggttgtacgaatagctttcgctgagacgctgcagcgtag | |
| | tttgtcgcccggcttcccgggtggataacccctcagactgagcctgcaggcccgccctg | |
| | gcggctttttgctgctgctcaaacttcacggcctgatcggccagctggttgagcttttgctg | |
| | gctggcaaccttatcgcccaggtcggccagctgccgcttgtactcgagcgtttcttctttgt | |
| | gcgccagcagggatttttcctgcgccgtaagctgacgacgcccagcggcctcctgcaga | |
| | acggtgaactgattttcagtttgccagagatcctgacgctgtttacttatgacgtcgttcacg | |
| | ctggtatgctgctcaagcgttttaagctgggcctgaagggtgagaagttcggcctgcgcct | |
| | tttcctcggctttgtccccggcgggcgttgagtagcttttgcctttcggtgtttttggatccttc | |
| | cactgcttttcaatcccggcgcgggccgcggcaatgtccttttcagtccacagcgtggcg | |
| | acaccgtctttcgcatcctggcggtttttctcaataagctgactgagctttttctctgctgaag | |
| | cccgcttttctgccgccgtcgcgccggactccaccagctggttaaactgctgctggctgc | |
| | ggattgcctgagcctgctggtccgttcgcattttttcccgcgcggctgccagcccttcctgg | |
| | gcgtattgctgatcggcaagatcgtaagcctgctttttcagctccacctgctggcgcgcgtt | |
| | tctcagcctttccgcatccgctttctgcagaacgttgttaccggcataatccgggtcgacctt | |
| | aagattgctggacagcgcgcggtactctttctctgctgcctgccactcagcaaaagagtcc | |
| | tggcgcttcatcgcggtgtcaggattacgcccgacgcccagcatcgcatcccacgcacc | |
| | ggaggcggcattcttcacccagttccaggctttttcgagggatccgagattatcctcgacc | |
| | gcaccggcgcgctgaatgaccgcgtcggaatatgcccgcatggccagctcggcagcct | |
| | tctgagaatcccccagcgcctgagcagaagctatctgttcatactgggtggctgtcagaa | |
| | aatgaagggaatcgttgagcgtcgcgaccgcgttaaccggatcatccttcaggcgtttaa | |
| | actgatttatggtttcgtcaacggcctgcccggtagcctgctgcagcctggcggcaacatt | |
| | gctgaccatgctgacgtcattaccgctgaacgcgccgctgccaacgacctgcgccagca | |
| | cgcctgcagcggcatgctgagtgatgccattacctgccagcgagcgcgccagcgcctg | |
| | cagctgccctgacgttttccccgcgtagttcccggtcaggatcagctgcctgttaaattcct | |
| | cagactctttgctgccgtcgtaccaggccttacccaacccgaataccgccgcggcaatcc | |
| | ctccgaccatgctggcgatcccaagaccgcgcagtgacagaagctggtctatccaccct | |
| | gcccggttagccagcgtgatcccggagccgcgcagcgcgccgaagttaccgcgcatg | |
| | acctcgccgatcagtattcccagttcctgccgggcggcggcactttgcagccccagacc | |
| | gtgcgtggcgactttggcagcttcgagcttgcggatatagacttcagccgcatcgctggc | |
| | accgacctgcgccgccttcatgcgcagtagctcggtaccggagagcttttgctctgcaac | |
| | ctgttgcttcagctggctgaggaatcgcgtgcgcgctgcggccgatttttcctccacgatct | |
| | gcagttctttttgacgggccgtggtgcgggaaataagggcgagataatcctgctgggttat | |
| | gttgccctgtgccctcgctgcgcgaaagcgcgcctgcacgttcgcaagcgactgtgtttc | |
| | accattgagctggcgtacgccgtcgatctggcggaaaaatgatgccgcaagttcatcctg | |
| | tcgacgggcaagcgcagcggcctgcccgtcattctcacgcatgcgctgattaagctcgg | |
| | tcacgcggcggtgagtttcatcaacggactttgaaacgttctgccagtctttggtaagccct | |
| | tccgttgcggccgactggcgggatttcatatctgcggcagccgccgcgccagcgtcacc | |
| | cacggttttaaacgcagccgcctgccgctctgaagcgcgctgcattcgcgtctggacttttt | |
| | cagagtcctcagccatcccggttagctggccctttatgcgggcaacctgctcactaaacgt | |
| | ggcgctgtcgacgtcaaggttgatgaccagatcgctaatctgctgggccat | |
| ECOLIN_10095 | ctaatctgctgggccatatcggatacctcctgttatcccctcagctgcggccatcagcgcat | 160 |
| | catcatccggctcgtcatcgctgatgacgataccggaaggagaaagcaggctgaaatgt | |
| | gcgggggtaagttccgggtcgcggaagaaaagagtggagatggaataaagcagctctg | |
| | agaaatgcgcatcgagctgagcgtcctgaaaataatgctcccggtagaactggtgccagt | |
| | cgcccagctcagtggaagtcattccagccagcat | |
| ECOLIN_10100 | tcagctcgctggcaagggcttttccgccgcaacgggttctgcgctttcggcctccgctgg | 161 |
| | ggcatccggatcggcagctttgtcatcctcaaccggaacgagcatgccggagagcagct | |
| | ttatttccatttctgctttaccgatcgcctccggcggccagccgctaagcacctgctggtaa | |
| | agcgtctccacatccgtgccagccggatcgttatgccacaaagacatcgcaatcaaacg | |
| | cgcaccgcagcgaatatttgagccaatcagcctggccgtcatttcctgatcgctgatgccg | |
| | tcgctgtcagcgctgacggccttttcctctgcggccataaacgtgatgtactcaatacgctg | |
| | aagcgccgacagctcgaagatggtcagtgattctttttgccaggtgaacttctcttttttcag | |
| | aaacat | |
| ECOLIN_10105 | ttacgctgcagttacggtaactttgcagaccgcaacgaaattaccgtcgctggtcataaca | 162 |
| | ataacgtcagcggtgcctgccgccacgccggtgacggtgatcgcattaccgctaacggt | |
| | gaccgttgcttttgccccgtctgaggttgccacacggaacgaggtatctgaggcactggc | |
| | tgggttaaccgtcacattgagcgttgtggttgcgccgacggccacgcttgccgtggcttta | |
| | tcgagcgtaacgcctgtcacggggatattcggggtcccgctttcttctgccagttccggctt | |
| | gccggtattggtaattttcgctgtacgggttatgacctcttttgccggaatggctttacccag | |
| | gctgctgcaccagccgcggaaaacgtcgacggtaccgttcgggtatttgattttgtaatag | |
| | cgtactgagccatcaataaaccatgcgacaaggtctttttgcccttcttcgcccggcttcca | |
| | ggcgagggtgaacgaggtatcgccagcagattttgccccctgggccgtcgcgttccagt | |
| | cggcatcctcgtcgtcgaggtaagtgtcgtcatacgattcggcggtcatttcgcccggcgt | |
| | cagctctttaattttcgccaggcggttccagtcgatatccgagagtgggttagcgaaagcg | |
| | ttgcccgttccggtgtaaagccagagggtggtaccggcacctttcacaggggccagcgg | |
| | gtttggagtaggcat | |
| ECOLIN_10110 | ttaaattgaataggtgattaagtacgtgaaatcgactgaaccccaggtggccatttcatcat | 163 |
| | cccgctgatagtcataaccctgcggggtgaacgtctcgaccagttcggtcagacctggg | |
| | atgaaggccattgccggatacactttctcttccatccaggaatcaagcgcgctgtcgggg | |
| | ctggaggctttaagaaatacctcgatgtgaacaaccgcctgccacgaatcttcgtcaagc | |
| | gaatcgccggtgtactccgcgtcagaaaggtatacagccacggcagggagatcctgctc | |
| | ttcaagaaaaacagggcgcccgtcaaaccaggtgaccgtgtcggtgatctcggctttcag | |
| | tttggccagaatggctgcacgaattgcgctgtgtctgttcat | |
| ECOLIN_10115 | tcatcgcttcaggtggatcctcagttggtttttcagggctgcggaaagttctttgggcatatc | 164 |
| | gctttcaataaggcgctttgaaatagcggtgaaggccacggtgagcggtgtctcaagag | |
| | gaactttgaccacatcaatcggataacgggcctgacctacgcgccgcatgacctgccag | |
| | cgcccgttcgcaagctgttggataaaagcgttacgaaaggtatagggcccgattttaagg | |
| | acgctgcccgctccgtttctggccccttttttacgcgagagcctgacgcgcgccgtgccg | |
| | agctttatcgcaggaagattaccgcggttgatttttatcgacgcgaccgggcgatcgtgac | |
| | gggccttgcgcagacgggaacgctggcggaccagacgaaccggaagcccctttttccg | |
| | gttatcatcaactgttgcttctttcgctacagctttgctcccctggcttatcgttcttctggccac | |
| | cctgttaagtgcttttgcggttgcctcaggaacgattaaccggctgaggctgttcaggttct | |
| | gaatagccctttccagtcctttcac | |
| ECOLIN_10120 | tcattcgagatggatgcggggttttccgttgaacatgtcatagcgggtaacgatcaggttct | 165 |
| | taccgtcgtagtcgacgctgtcgtttcggcgtggctggtaaagctcagagaaaaccacca | |
| | gcgaagtacctgttcccgacaatggccccatttcctcgagttgctcggcgggaacaacgt | |
| | catagctgctgccattgatgatcgctgtctttcccatcttttttatagtggccgcgtccat | |
| ECOLIN_10125 | ttaggcattgatcttaacttcaacaacggtggtgtttgcccctgcatcttcccaggcgatgcc | 166 |
| | cgcggcaacggcgtccgtttcttcgatcgtgattttgccgtccttcagatacacctgcgccc | |
| | cggcagtaaccgcatctgcggatacttttggcaggaggaaaacaccctcagtaaaaccg | |
| | tccccggtatcgccagccgggatatcggtaattgccaccgcgataagttttccaacaaca | |
| | accgggtcgccgctgtgaacatcggttgcaccactgtttaccagagggatcgttttcccgt | |
| | cctgcgcatagttcttagccat | |
| ECOLIN_10130 | ttactgaccagaggatttggtcatgccgcgatagtccagcggcgccacgccagcatcaat | 167 |
| | acgcactttcgtggcgataccatcagtggtgaagccttcctgctgatcgatgtatggcgtgt | |
| | cgacgccgttgagataagcgacctcaatggtgtcggtgcccttcgcggcagccagatac | |
| | caggctttcgcatcagcttcatccagacgtggttcggcaatgacttctgcaaagttctggat | |
| | agggttaacgatcccggcattgatgtctgcacctttaacactggccgacttgatggtctgat | |
| | ttgccagagtttccagggcgacgggcaccagcatgtaggccggacggatattcagggtt | |
| | cgctccccctccttctgcagacgcatcagcttgcgcgattcgtccaggctggccacagaa | |
| | attgcacccgagctcaggttcttgtgatcggcatggaacagcgcctttccgtctgagagttt | |
| | cgggtttttggtcagaatggcgtaaaccagatcgccaatcgttgctttcgccgcgcgcccc | |
| | atcttcatcggtacgtcggtaagctggttcagatcgtcgttgatgatcgcctggcgagttac | |
| | tgagaagatttcaccatacgtggcaagcgcgatggtttcgcctttgtcactggtagtgatgt | |
| | acttgtactcagccccttcgcgaacctgtcgcagagaagggaacccacccataccgaca | |
| | cgatgcgccgttttgaagtccgacagctggccttttttggtccactgctcgaaggtttcctgc | |
| | gcctcgtcccagccctgaatcagcgctttgttcgcaacatcaagcagaatgttgccaaagt | |
| | cagaggtgctgtgggtcagcgccaggccaaccatctgcatcgggttgtagctggccacg | |
| | ccgatacctttttctgtcagggccatacgcgcatactcgcgcagcgtcataccgttataaac | |
| | gttatcccgctcctgaccttcgaacccggcacgcgccatcagtgcctggcgaataccatc | |
| | cgcgacgaagttaccgttgcccgcatgaatatgcggctgagtggttttattggacggcgtg | |
| | gccgttttaccgagttctgccagcagcaaatctttcgccttatcgacggagcaatcagggt | |
| | cggccacacactgattctgcagttccatgtgcttattaccgaacatggcaaagagatcgcc | |
| | gatagcgttaacacgggttttctgctcagccaacacctgcgcgcggatcgcattttcatcc | |
| | ggtgccgggtctgtttttgcctgcggtgcctgaggctgggtaataaccgggtcacgctgg | |
| | gtagtgttgcgcggcggggtgatcatgttgcgaatgctttttggcattttttcaaattcctcaa | |
| | tacgttttgaatgaatacaggccatagcctgaagggatggtgtcacctggtcggcaaaac | |
| | ccagttcaaggcactcgctgccgttcatccaggtttcgtcctccagcattaccgcaatttctt | |
| | cggtggattttccggttttctgtgcataagccgggataagaacggattcaaccttgtcgaga | |
| | agatccgcatagtcgcgcatatcgctcgcgtcaccaccagcaaacccccagggcttatg | |
| | gatcatcatcatcgtgttttcaggcatgatgaccggattgcctaccatcgcaatcaccgag | |
| | gccatggaggccgccagaccgtcgatatgtacggtaatcgccgcgccgtggtgcttcag | |
| | cgcgttataaatagcaattccgtcgaagacatcaccaccgggcgagttgatataaaggttg | |
| | atgtgggtgacgtccccaagtgcccggagatcattgacgaactgtttcgccgttacgccc | |
| | cagtacccgatttcgtcataaataaaaatgtcggcctcactgttattgctggcctgcat | |
| ECOLIN_10135 | ttactttttgcgctggctttcggacggtggcgcgcccggttctttggcttcggcactggtgc | 168 |
| | ctcctttatcattggcggggtcggtgtcaaacaccaggccctgttcacggttctcgtcaacc | |
| | tcagctttacggcgtgacttaacatcatccgggttgcgaccgctggcacgtatccagtcgg | |
| | attcagtagcagcaccgccgcggatctgcgttttccaggcattcgcttctttaacgggatca | |
| | atccacggcataacgggccccgaataaaccgcgttataaagcgagtccatatcaatgcct | |
| | ctcggcagcttgatttctccggcagcaatagccatcttgagccaggctcggtacatgggc | |
| | cgggtcactgaaccgatgaaccagtcctgaagaatcagatagccgtcggttgactcgac | |
| | aagctcctgccgctgggcactgtacgttccgttgtagtttctggatgtgctggaaaagctga | |
| | ggcgactgccggcggacacggcacgcagctgtccgttacgaaaagattcgaggttagg | |
| | gttcgggcgatcggatttaatcatcccgatttcttccccggcctgcagttcgtcatagagca | |
| | taccgggctgaatcatcagctcgcggtcatcgctgcttgaatcagaatcgaagctctgtcc | |
| | gtcgccttttttgatatacatgccgagtgccgcagcaattctggcagcagtaagctccgagt | |
| | cctcgtattctttcagcgcgctcagacgcatcagaacaccagacaaaagagacgttccgc | |
| | gggtctggtgcaggcgtcgggtgaatttgagatgcagcatgttctctgcatctatctctttg | |
| | gtatcaaactgacgcccggatactggcaggcttttatagacctgatattttttcgggcgtcc | |
| | ccagttatcgacaaaaacgccctgattgagctgggtggcagcatcgctgttcatcggcac | |
| | aaagtccggctccagcgcttccagccagaacggcacgccagcaaccggctgaagacc | |
| | atttccggtaccgcgaaccagctgagcaaatacctcaccgtcccggagccacgttcgca | |
| | gcatcagccgctccagcattgggcgggtaaactgggttgtgacatctggccttacggacc | |
| | attcgccccactttcggcggatatcagtggccagctttttagcgatcttcccgttactcagca | |
| | tcggatgcggttcaactatgatgcccttcgcacccaccaccctttcttccagcttgtcgaaa | |
| | acgccgatcaccagatcgtggttgttatccagccagcgcgcctgctgcctcagcgaaac | |
| | cgcccccatctggctgagctgatcggctgaacgattttccttctgggctttgtgggtacgcg | |
| | tttgctttaccgcctcatacgctttaataactgcgcgggcacgcaggcgtgaggctttccag | |
| | cctggtgaaaacaggccaatcgcatcatctaaaaaactcat | |
| ECOLIN_10140 | tcatccaaacctcgccagcctgtagccgggtcgcccacggcgtttgttattgagcgttgcc | 169 |
| | agtcgtcgctcccattcctgacggccttttctgatttccgacaggttttcgagcgtcatctgct | |
| | gcccgttgaaagtgattgatttcccctccagaacagacagctcggctgcagcatagcggt | |
| | cgatcatgttttgaatatctgctggattcac | |
| ECOLIN_10145 | tcacacccaacctcctgacgaagaccacggattagcctgctcggttacgggcttctcacg | 170 |
| | ttttggttttggtttagatttcggcgcaggcggcggggatggcatttcgccagcttccgtctg | |
| | cgtgtcctcgatccacgtttcccgccgtgcccactcaggagctgacggccatttgattttttc | |
| | gtaaccactaaggatggcgagcgcgtcggcataaacgagcaggtcaaatgcttcgtttg | |
| | cgccccggccgggcttactccatttcccttcattcgagcgttcctcatacgtcagttcgtcat | |
| | agaaccagctgcccagccaggcggggaaatgcacatagccagggccgggtgaatcac | |
| | gccacagcgcattattcacccggtctttaagggcatcggtctggagaagataaagaggca | |
| | catcaccagtcgcctgtgcgcggcgcgttgatctgcccgtgttgtcgggaaacgttcgct | |
| | ggataagtttgctgcgcctgacgctgtcccctttgaagagatagatacgcttacccagccc | |
| | ctcacggcgacatctgcgccagaacttgtaggcattatccgtcacgccatcttcgccccct | |
| | gagtccacggccatcgacatcagccgcatgccctttgatgggtcagctgcgagcggcca | |
| | cgttttatcaaagacgtcagtgagtaaaagatcccagtcctccggatagctcgccggatcc | |
| | acctgaatgctttcaccgttgccgtcgcagcgcagcgaatgccggatgttgtaacggtca | |
| | actatccagcgctcacccatacttccataacccgtaatctgcacaacaaagcgccggttgc | |
| | gcccggcctgcacgtccacggtcgcagtgagaaactgcacgccgttcggtaccgaacg | |
| | ttttgggacgtcttcggcacgctgctcgagcaattcacttttacgctgctccatgctggctcg | |
| | cggcaaatagggcctgccgaaatcggtgttgatcaccgtcttcagggtttcttcgctgcgc | |
| | gtggattcatattcctgctcggcggtcagaaacttataaataagctgcgcccaggtctggta | |
| | agcagctgccggaccttccatccagaaggaggcaatacgggaacgacggccatcacc | |
| | gctaaccaggcctttcctgtcgatggtttgcccgtcccggagccagacacatttcatgttaa | |
| | gcgcacgcttcatgtccggtgtgatcctgcctttacaggcagggcactgaagaaacgcc | |
| | gcttcgctggcaagcacaggatcgctgctgtcgcggtatccggtcatattgtccatttccg | |
| | gctggaaatattcgccgcaatgcgggcatggccagtaaagacgacggcggtcaccacg | |
| | gttatagagcgataaaattccggtggtcggaggggcttcatggggcgtggagcgccgcc | |
| | attttgtgtctctgatatccctcccgggcgagctctcaaccagcgtcatcccggaggacat | |
| | gaatgtcgtggttcgtttcgatgccagtgaaaaagcatccccctccccgtcgatatcttccg | |
| | gaaagcggtcataatccgtcagcgccacacttttatagtccgaggacgacatgatattgac | |
| | ggatggccagcccagcttcagatagttaccggcgcggaatgtacggtcgtagacgttgtt | |
| | atcgttacgtcttgggcttagccgggttttaacttcagggctacagcgaaaagtacggtcc | |
| | aggcgttttttggaatgctcgcgcgctttttcctcagatacctgaattacaagcatatctgcc | |
| | ggatcgcagacaatgttataaacgatccagccgtcaatcagcccgatggttttacccgttc | |
| | gcgctgggcccacaaacacaaccgcatcgtattcacgcgatgccagacagttcatcggc | |
| | tcaatcacatagggtgccagatccggatcccacggaactgagtttcccgcccccattggc | |
| | acgcgcatataagtactgaccgcatcggccaccggcatacgacgcggggctcgtaaaat | |
| | accggaaacatcgcggcggatgtccctggcggatgcccgctttgccat | |
| ECOLIN_10150 | tcagtcctcctcaggctgctcctcctcttttccagcgtcctgcaccttctccgccatctggtc | 171 |
| | gcgcagatcatcgataacgctttgcacacgaactaccgcagcaggcgttaaagcacagt | |
| | cgcgctcgagcacatccgggagggtttcaagtaccatgacgacggctttcgccatcaatg | |
| | agaattctcgcgccacttcatctgcgggtattaactgccccgtatcctgttcgaacttcagc | |
| | ctctcgttctctgctttccagtgggacagcctgtcagaagggggcatatcgtcgatgttggc | |
| | cgaaacggtagggatcatcagttcggtcagaatgtcggtcaccagatagagctttaacttg | |
| | ctattgctgcctggagcaggttcaacatttttcagtctcgcggcaaccgtctgacggtgtac | |
| | gccggttatccctgccagctggttgatattgagttttaaagtggcaatttcctggtccat | |
| ECOLIN_10160 | ctatcgacggcactgctgccagataacaccaccggggaaacattccatcatgatggccgt | 172 |
| | gcggacataggaagccagttcatccatcgctttcttgtctgctgccatttgctttgtgacatc | |
| | cagcgccgcacattcagcagcgtttttcagcgcgttttcgatcaacgtttcaatgttggtatc | |
| | aacaccaggtttaactttgaacttatcggcactgacggttaccttgttctgcgctggctcatc | |
| | acgctggataccaaggctgatgttgtagatattggtcaccggctgaggtgtttcgattgcc | |
| | gctgcgtggatagcaccatttgcgatagcggcgtccttgatgaatgacactccattgcgaa | |
| | taagttcgaaggagacggtgtcacgaatgcgctggtccagctcgtcgattgccttttgtgc | |
| | agcagaggtatcaatctcaacgccaagcgtcatcgaagcgcaatattgctgctcaccaaa | |
| | acgcgtattgaccaggtgttcaacggcaaatttctgcccttctgatgtcagaaaggtaaagt | |
| | gattttctttctggtattcagttgctgtgtgtctggtttcagcaaaaccaagctcgcgcaattc | |
| | ggctgtgccagatttagaaggcagatcaccagacagcaacgcgccacggaaaaacag | |
| | cgcataaagcacttcattagcagcgccagatagcgtaatgattttgttactcat | |
| ECOLIN_10165 | ctatttgtgggtaaagttcgtagtgcgctgatcgtgcaaaatgattttagttgggaacagttc | 173 |
| | gcaactctgtcccataaaaatcagcatattcccatctatcccatatccagcgcattgaccat | |
| | cgggatactgaagggagattccatcatctcttagaaagatcaccatctcttttgtttcaatttg | |
| | catatagctacctggaggatttatgaatgcaaggattttcat | |
| ECOLIN_10170 | atggactattaccatgagattgattttccatctttattcgcgagagcagtggaaagcgatga | 174 |
| | cgatgtgggtactacattgcgcattcacctactttgtgagcgcatggtcgaagcatggatat | |
| | gcgcatgctgtgactgccaagatctctttggaagagataaaaacaaacttttaatcgaatgt | |
| | aatactaaaatatccatggcgggaaacctgggaatccccccggaacttatgaaatcactta | |
| | aaaccatcaactcaatgcgtaatgaccttgcacacaatccatcaatacaaagcattgctgat | |
| | tcaaggatccagagcctgaaggatactctgactgaatactttaaacagcatccaacggaa | |
| | cccagcatggaagaatcaaaactgggtatttttaacgccgagaatcaattaaccgaagaa | |
| | gtttccttagatagtgacagttcaaaaaacagacttaagttaatcttgctgttcagcaagttaa | |
| | tgcaggcgttaatgcaattagttgcagctaatcataatgggcgctgggataaccaatttagc | |
| | caattcgtttaccatgtgaccatgaacgcaacaaagagataa | |
| ECOLIN_10175 | ctagtcgtcgagttgcaacacaccgtgatccagtgattctgaataggcgataagtccggta | 175 |
| | taaccggggataatctcaccattatcagcttcaaattcaggaattgtgccggtggtgatggt | |
| | gtattgaggctggccatcttccttcgcgaaggctgccaggtcttcaatctgcttagctgtaa | |
| | gaactactgtcat | |
| ECOLIN_10180 | ctattggttattcgacagtcgcactgattcgtaaatccgctcacacgtcattcctgcccggta | 176 |
| | gctttcgtcagatcgtccagcataatatcgagctgcttctgcaaggcttccgagcatgtcgg | |
| | caagcattgctgcgttggctccggctgttttgcttctgacggaagtggcgagatctgcggt | |
| | gtgctttgcggcgtccatgtgggtagcgagttttgttgcttcggcgcgcagctgcttaacag | |
| | tggtagccaggccagcagaagtaacggcagcgctcgctgcttgagcttgagcatctttaa | |
| | cggcctcatcccgggcgattgttcgcccttgttcaatcatacgagctgcggtctgtgcattc | |
| | gcttcctgtgaagattccgcgctatcccggtcagcccactttattttccagctgcggttcgtc | |
| | cactcactgccagcgagaaacgaacctaccaacgcaacaatcacaat | |
| ECOLIN_10185 | tcactggtctatcccccagcacgtcagtgcgctttcctggtcccgccgttctacctgcccat | 177 |
| | aacatccatccttctggcccttggtcaggcggcaatcgcggccaccgtctttaatccacca | |
| | gcggatagcttcacaggctcctttcgtatcgccagcattaattcgcttatagaacgtagacg | |
| | ggaaacattttccggggccgatgttatatgggcagaaagaagcgatacccgctttctgtg | |
| | gttcggtcagtggtactttgatatttcggtcaacccacgccagcgccttgtcgcgttcaatg | |
| | gcgtttacctgggcgcatttctcagctgacagcttcatgccctgtactactggcttgccatca | |
| | accattgttgcaccacggcaaatggtccagagtccgccgccgtcgcgatatgctgtcaag | |
| | ctgttaccctctttctcatccagaaactgatcgagaatcacgggtgcggaagccccggca | |
| | agaatcaaaccaacgaccgctgcgctcaatttattcttcagctttagagacatagccat | |
| ECOLIN_10190 | ttattcttcagctttagagacatagccattgcgccgatcctcccgttctttccagcggaaata | 178 |
| | ccagttcactgcacaggtgattaccgtgcatgcgataccgacaataattgcccagtcgctc | |
| | aggcttaaccctgcaattctgtcggccaacatccaggacacctcttttgctgttttagctgttt | |
| | cggcatatgccttcgctgatacaccgcagccggcaagcgtggttcctgatccatatgaaa | |
| | gtctgctgtaaatggtgctcattctggtcat | |
| ECOLIN_10195 | ttatgattttttcagtttttccacctcttcggtggtctgtataaacctgtctgcctccagttctacg | 179 |
| | ccgatcgcccgacggccaagttctattgcagctttcacagttgaaccagagcccataaag | |
| | aaatcggcaacgatatcccccggtctgctgctggcgctaatgatctgtttcagcatgtcgg | |
| | caggtttttcgcatggatgtttgcctggataaaactgaacaggcttatgtgtccatacgtcgg | |
| | tataaggaacaagagcggaaacagagaagcagcgccgaaggattttgtattcctccagc | |
| | aattctgaatacttgcggtttaatgactggtaggtagccaccagctggtggtgaggatgttc | |
| | aagcttttgctgaatatgtttatcgatggcgatccgcgtgaacagttcctgcaattttcgatag | |
| | tccacttcattcggtagttgccattggcttgcaccaaaccagtgtgacgccatgtttttctttc | |
| | cggttgcctcagctatttctttcgagctgacacccagtgattcacgggcattacggaagtaa | |
| | tcaatcagcggcgtcataatgtgctgctttagctctgtgcttttcctttcgtaaacatcctcttta | |
| | cctgtatacggtccaagatagtgctcagcaaacaaaatccgttccgtagatggaaagtac | |
| | gcacgcaggctttctttattacatccattccagcggcccgatggttttgcccaaatgatgtga | |
| | ttcaaaacgttgaatcgggcgcgcatcataatctctatatctgaggccagtcggtgaccgc | |
| | aaaacaggtagatgctgccagcaggtttaagaacgcgagcatactcagccaggcagcta | |
| | tcaagccagcgtaagtagtcctcgtcccccttccattggttgtcccagccgttgggcttcac | |
| | tttgaagtacggaggatccgtaactataagatcaatagagttatccgggagggtggcgac | |
| | gtaatgcagactatcagcgttgattaactcaacactgtttatttttacagtatttttcat | |
| ECOLIN_10200 | ttactggaggcgtttataacatccgaactggtaatcagataaccccgccatcaccagctgc | 180 |
| | gtaagtatgagctggcaacgttcgtggctgaggtgggtattctgtgcaatctccccagccg | |
| | ttgctggtttatcgcttaattcattgaaaacagcctttgccgtttctgtcatatcttcctgatttag | |
| | cat | |
| ECOLIN_10205 | ttagcaaatattccacatcatcgtactaccgttatggttttcgataatttttgcggctgggctag | 181 |
| | taccaaaagagtgcatatagcaatgatgaatagtaaggaccagatcctgcaacgtttggtc | |
| | actctctagctccatgatatttaaaccaatattttgagctttgtccaaatgaatatgtctggcat | |
| | gtgcatacgttgcttggtggttgtttaactcatcacatatacgcttagccttagcttcagcgtc | |
| | agcctgacctgcgaacataccagtacaaagccatttctggacaatttcgttcgcccagaga | |
| | attgctttttcacactcgccaatcaacgttggatttagtttttggaacgtaaattgccaccattg | |
| | cagtgcagcagggttggcaaaaatttccgcttttgctctctcatactcctcaataattgcatg | |
| | agatgataacccattaaactgtggatcaattggccccaagttcgactgtttacctaaaacga | |
| | tctgctcagcacaacaagcaagcattgtgccacaactcattgaaatcataggtacaatcgc | |
| | tcggatattggttccgaactttgaacgaagataatgaccaattgattctagagctgcgatatc | |
| | gcctccaggagtatggagtaagatatccaatcccagactcgtatctaacccattgatagca | |
| | gacataagaccatttttatcatcatctgacatctggatcagatgttgaaacccaggcccccc | |
| | tttttgaaggaagcctgagtaataagaaattacatttcggccagtatgtttcgataaatcacg | |
| | taagtacttgtggcgaacctcatccgctggtgtacgttgagcgatagtacccatctcaccc | |
| | aatacgtctatccaatttggcat | |
| ECOLIN_10210 | tcagtatgagtacagttggtgagattgctgaccgttctgctcagtagtatttggtgttactgtg | 182 |
| | ctgtatgaatagagcacaccacttctcacattcagatcgttttgctgagcgagaacacgcat | |
| | agcaaaatgctgtacggattcgcctttttgaaactcttggggttgtatgcccattttttcgtaa | |
| | aattcagcagcgctcat | |
| ECOLIN_10220 | tcacatttctgccactttgagggcttcttcttcctcatagtattcaagagccatggccaacgc | 183 |
| | agattcatcaagctgggtaaaagcggcctttaacccagcccagtgccctgaatagacacg | |
| | caaccatgtcgaacggtcaacgctaaccatgcgggccaacgctgcaccagcatagtcttt | |
| | ataggtttcattatttctggttgcggcaatttcctgccctgccagccataccaggcctatcag | |
| | tttctttactacgcgctcctgaagggagttatcacccaggcatttctgataagttttccagac | |
| | gtattcacacatcatcacctggtgcttatagctaaggtcaaaaccgtagcagtaccgcaac | |
| | caggcctgctggtatccactaagcgcggacactgccctacgccacggcgcggactcaa | |
| | attccgcatcttttatcggcggcattggcctgcggcggctgcgtgtttccagcacatacagt | |
| | ggcgcggaaagtgagttaacaaagcgtggccccttctctccttcgagttcgacgagatga | |
| | attccacggcgaggggtggcatttttgtctgctggtgggtgttcactgaaagcctcaagct | |
| | gcccttttgttcccccagacaggtcaggtagcgcgcggcgcaattctattcttacaaaattc | |
| | aggtcttgttgattcat | |
| ECOLIN_10225 | ttaagctttcgcaattacgccgatcgccagcgcccgatccataaaacgcagtagcagctc | 184 |
| | aagctgcgtaccatgcttctgctcgaatgccggtacatcggcgtgtaactcgtcgtggcac | |
| | tctctgcacagagggatcacgaagagatcatgggcttttgttgctgtcccccccataccgt | |
| | gccctacgatatggtgcggatcatctgctggccgtcggcaacactcacagggttgtgtttt | |
| | aacccagcgggtgtacgtctcatttatccagcggcgtcgctttggcctgagcatgaaagat | |
| | tctggagactccggatcaacagagagcgtgaggatcttcttcgccttctcctgcacgagg | |
| | ctggttgcagaagcggaaggcacaatgtcgctttccctcatgaccgagcggatcttatcat | |
| | ccggaaggcgtagccccttgtgcgcaacgctttccggaataacatcagccaggtcgtttc | |
| | tgaccatccaccagcacagttccggaagcgtcaggatatgcgactcgggaaaaccaga | |
| | atcacgccgaatgacttccagaatccaggataccaggtttcctgccgctatacctgcaagc | |
| | tgttcggtatgctgccccgacaaagtgtgatcgcaatgccagcacaggcgaatacttcct | |
| | ggtgggtgccgcattgttgtgaagttcttgtcgtgccacgatgaatgtggccactggcattc | |
| | aaaccgattactcaaccattgctcaagggaaggaagcccaccagcacgctgaataaccc | |
| | gatcattctcgaagacctgccgcattaccggatcatcagccagcggctgaatggcggcg | |
| | ggaacagctcctgtactgaatgacgccatttcttctggttcaggctcaagcagaacgcgac | |
| | cgcgcataaagaggtgcatcagttccgcgccgggacgaaacaacacaatccccatacg | |
| | atgggcgatctcgggggtaagcagagctctcac | |
| ECOLIN_10230 | tcacgcgacctgccccctggcaatgtgttctgcccacagtccaccaatccagcgcacgc | 185 |
| | ctttcgccgtgaaacgtgcctggctgaatgcatgatttgaggttacggatgtgccggttttc | |
| | acttcaaaacggcccgcatcaatatgctgatgccgtggggtcatcgttccgccaagacga | |
| | tacatgatgtcgttctcaaggaggaataaccgcagatcgggctctttggccttaagcagttt | |
| | tgccacctggcggaatgacattgacccactggctgtacagtaccgatcaacaaacgctac | |
| | cttcggcgccgcggcagccagttcgttagtcaactgctgtttttgttctgcaaggtcagctg | |
| | caagacgtagggcttcagagaatgattgaggaatcgtctgctgctgtgcctgctcaagctc | |
| | ctgccagcgatcaaccagacgcgcggtaaactccggcgacagctgcgcgacaacgat | |
| | ataactgtcccgcttccctatcagataaaccgataccgactgattgaggtgatttttaacttc | |
| | ccccattggggggagttcaataacaccgcgctctgccaggcgttcaatggaccgtttaac | |
| | atggtcatgtcttgattccaccagctcagcaatatcgctgctggacatggttaacgctgttgt | |
| | tgctaactggctcat | |
| ECOLIN_10235 | tcaggcggctgcacccgccggttcatatctgctgattgttatctctacccgacctttcggca | 186 |
| | caacgggtccccattccaccagcatgcgcttaatctggctgtcgtcttcccagacacccgc | |
| | atgcgtcagcgcgtcaaacagggctttgttgtaattatcgatatcccggcggcgcgcatcc | |
| | ggcgggtacagagtgatttctaccgctgccagttcagtcgatggcttcgggagacgtcgt | |
| | aattgctcaatgatcgccacgcaggcagcgctctggtatttacggccagcggcgctaatg | |
| | aggtgacgaccggccagcggccccttgttaggggcgcgccagtaagtgttcacgctcg | |
| | gaggaaaaggcaggatcagtttcac | |
| ECOLIN_10240 | tcacgcggcctctccccgcatattgcgaacaagttcagaagcagcagtaatgatttcgctg | 187 |
| | gtggcagtccgctccagccagagttgattgatattggctttcagcttgtgttgcagtgactc | |
| | atccagcatgtcagcaccatccacctggtcgaatacaattctaacctccagcggccagat | |
| | acgggactcgggaagcggatccgatactggtttagctttctcacgaatgtgcatgcggat | |
| | ctggcgaatattggaccaactggaaacatccaggcttcccatggctgcaatgaagtcagt | |
| | gctgttcatgccatattcaccggatgcttcaagggcaacagtgcgaatacgttccgacata | |
| | tccaggcgcacagcagcgtcatcgaattcaatcgacaacagccactcatccacaccgaa | |
| | caaaatactctcacgaataagcagcttcgctttgtcgatcgttaaaggtgatacctgagtga | |
| | attccggtgcttcgacagaatccgccgcccaggtatgcccaaacttcgattcactgaatgt | |
| | gtattcttctttatcgccaaacgcagctctaacgcatgcccacgcctcgacaccgctgata | |
| | gcaaaaatatctttctgggtgagtggcaactctgcttctggcttatcagctgcaggaggtgt | |
| | ggcagttacaggttgagacttgctggcagcaaattgtgccaaagccataaacgcccgcc | |
| | cttttgcctccagttctgtgcggttgatatagctgaaccgctcgccacgccatgacttatcga | |
| | atacagctatggcaccggcaaaaaacgcgctggtgggtttctgtttttcgtcagcaggtac | |
| | aaaccacacaggcagatcgaacccaatgcgcccgcgaatgaatacaatgtgatcggcat | |
| | cttccggccaccacgtttcactcggcgcggcttttatcaggaatacatagcgaccgccctt | |
| | ctcgcgctgggctgctgcgtagttcatgatgtgcgtcatgccggtgatcgcctgcttctcgt | |
| | ggtactgcgaacggctatacggtgggttgccatagccagcgccacccagttcagccaga | |
| | cgttcagaccagtcctgcgtcagcgcgttatcttcggcggtgtaccatgccgggcatttcg | |
| | cgttgtcgtcgtcagcaaacaaatccagaactaatggaccaaatagcgcgttgatccccc | |
| | aaaaaagcagatccggtgtccgccactgatcgccaacctctttcaattcgtgagctggttt | |
| | gctacgcagtgccgccagcgcctggcaatatttattggtcatcat | |
| ECOLIN_10245 | tcatgaacggaaccccgaattttctggcagtgagtaatcaacactctggaagtttgcgcgg | 188 |
| | ctggctgagttagtctcccatttgccgttaacgcgttcaggccggccagcactggaccattt | |
| | ggtcgcgctttgcaggtaaccagggaagttttttggaatgaacagagttgccgggcggag | |
| | gtattgcgcctgctcgctatcacgccaatcggcatttttgtaatccactaccaagcacaggt | |
| | catcaacagtgaattgttcccgaagacgggcgcgaatattctccagcgacgtgctgcata | |
| | cctggtagcgtgagccagtagtctgattcaggtaagacaaaacctgtctggcctgatcagt | |
| | aatcacaacctcagggtcgggttgcgccgcaaccggacaagagggttttgaagttacttg | |
| | tggttcttgttttgattttactgacggatccccaccagattctgacgggtcaaaaccgccttttt | |
| | tgctggatttcgacgcctcaaattttgacgggtcggattttgatgcatcagattttgatgggtc | |
| | agattttgatgcgtcagattctgacaggtgagaaaaagcagcctctcgcaacttaacgacg | |
| | ttaagctgatatacgttcgatgcgttacggttcccattacggcgctgcttacgggaaagcca | |
| | gccatccttctcaagctgagcaagggccgtccttaccgtactttctccagcaccgatttgac | |
| | gtgcgatcgtgccaatagacggccagctaacaccctcatcactactgaagtcggccaga | |
| | cgcgccatgatggcaacgctggataacttcatgcctgacgaagcgcatgcatcccatac | |
| | gtaaccggttaatttagtgctcat | |
| ECOLIN_10250 | ttaattctgtaaatttacgctggaattgttcaagagggctgaagcactcatgatcgtaccctt | 189 |
| | cgcgaaggtatataacgcgctgtgtatctggctcccagcggacaactctgacgggaactc | |
| | cgtagtgatctctgaaccgccggttaacttcagccattcctcgcgccccttctcgttcatctg | |
| | aacaaatgcttctaccatcaagtctgctggctggtagttgcctccatcagccgcgttatttat | |
| | gatttccacatagccgaactgggcatctttacccaccagcggcaaacatctgaattgctta | |
| | gctggtctgaatcggtttacactgttcat | |
| ECOLIN_10255 | tcatgcgttagtttctccactgatacgacacgccaaggcgcccggagctgcacactcgcg | 190 |
| | ggcgtcaccttttctgcctgttgaaacgaatacgtcaatcgcctgatctgaaacaccaacc | |
| | ccataaagcgccataaatcccaggaacccgtgaatctggtggcggagcttcttactgaat | |
| | aattctgaaagcattttgcgctctgatgaatcaattaccccatcagccgctgctgccatcttg | |
| | gcattagccagctcaccagatgctgctgccgctttcatctcaatgctgtacagctcaacgtt | |
| | atccaggctctcagcagttggaacatccaccagccatttcccttttcggttcgcctggtact | |
| | cggccaagtaacaagaaccagacaggtcctccatccgttccagttctgccaaggtaaag | |
| | aaccgactgccacacttctggtacaggtggttgtggaactggtcgatagtcatccctaaat | |
| | cggaagccatacctaagcgaccatgcttgtgtgccttacacatcaggcggattgctgtattt | |
| | atgctgtctaccat | |
| ECOLIN_10260 | ttaaagttgactattgttgttagcggaaggtatgccgtcatttttgttcggataaatatcaggtc | 191 |
| | gtaattgatggggagttactacccatccgccccattggcagagttgaataactctttcagaa | |
| | ggtactcggttctttgcaatccagttcgcaacagattgaactgattggaattcaaaccgcctt | |
| | gatacctctgaaatcgacccgatcgccttcacagctttagctgttacattcttgtgttgagat | |
| | gacat | |
| ECOLIN_10265 | atgccttgtgcgcttaatcttctacttatggtggaaaatgctaaatacaaagactttgccgaa | 192 |
| | aggctaaacaggtctctccaagagcaatctattggagttaaagaattgtcagagttcagtg | |
| | gtgtctcgtatgagatggcgcggcgctacactcttggtactgcaaagccgagagatgaga | |
| | agatgattcgaattgcagaaagacttgccgtctcaccggcttatcttgattatggtgtgcctg | |
| | ttaatggtggcgacgcgccagccaaaggcacggtcagaatagagcaattggatgttcat | |
| | gcttcagccggttccggatatataaaccaaccattccctacaatagtgagctcaatagaga | |
| | ttccagaagagaggatcttcgagttgtttggtcgtagaagccttgatggcatcgtcatgata | |
| | aatgttgatggcgatagcatgatgcccacgctttgcccaaaggacctgcttttcatagaca | |
| | gcaaggttgaacaattcagcggcgacggcgtttatgtgttcaattttgaagacagtacgttc | |
| | gttaaacgtttgcagaaggtaaaagggcgccgactggcagttctttcagacaatgaacatt | |
| | acccgcccttcttcatagaggagcatgaaatgaatgaactatacatattcggcaagctaat | |
| | cagatgcttacctctaaaaatgatagagtttggctaa | |
| ECOLIN_10270 | ctatttaaagagcttcttcagcttgtcctcaaccttcctgatttcggaagtaagctggctgctg | 193 |
| | ttgacattgatagtagctccacatcgacaagtgaaacttttgttcgacttgagccaagcgatt | |
| | ttcttcttcgtcttagtgccgcacttagggcatgcgggtaacgtaatttcctggttatcaaaag | |
| | cgcccat | |
| ECOLIN_10275 | atgaataatccgtttttcaaaaatatgttggtgtatcgcattagtcgcgatttcaccatcaacc | 194 |
| | aggaagagctggaacagcagcttgaactatttcgcttcactccatgcggtagccaggata | |
| | tggcaaaaaccggttgggtatcaccacttggtcagctgtcagatcgcttgcatcacactgt | |
| | caataatcaagtgttgttggttattcgccgggaagaaaaaatactgccatctcctgtcattac | |
| | tgaagaactgcgcaagcgtgtgtcgcgtctagaatccgatcaggggcgtcgcctcaaaa | |
| | aaactgagaaagattcgctgcgtgatgaagtgttgcactccctgcttcctcgggcgttctcc | |
| | aaaaactcgactgttggtttgtggatcaacgtcaccgacggtctgatcatggttgatgcag | |
| | ccagcgctaaacgtgccgaagactcactggccctgcttcgtaaaactctcggttctctccc | |
| | ggtggtaccgctgactatggaaacgccgatcgaactaactatgaccgactgggttcgttc | |
| | cggtagtgcgcctgctggctttggcctgggtgatgaagccgaactgaaagctattcttgaa | |
| | gatggcggtattggacgctttaaaaaacagactctggtcagtgacgaaattcatgtgcatct | |
| | ggaagctggcaaagtagttacaaagctgtctatcgactggcaacagcgcattcagttcgtt | |
| | ctttgcgatgacggcagcatcaaacgccttaagttctctaatgagattacagaacaaaacg | |
| | acgatatcgaccgtgaggatgcggctcagcggttcgacgctgactttgttctgatgaccg | |
| | gcgagcttatctctctcattaacggattaacaacctctctcggcggcgaagccaagcgata | |
| | a | |
| ECOLIN_10280 | atgagctacattcagacattatccggcaaacattttaattacctcgatatccaacaggacga | 195 |
| | tatcgtgatcgaggatattgctaccgcgttgtctcatatctgccgctttgcagggcatcttcct | |
| | gagttttacagtgtcggccagcatagcgttttaaccagccacctcgttccgcaggagtttgc | |
| | attagaagcactgcttcatgatgctgctgaagcctacctgcaggacatcccctccccactt | |
| | aagcgcctgcttccggattaccaggcaatcgaagctcgtgtggacgcagccattcggca | |
| | gaagttcggtctaccaactgagcaacacccaaccgtgaaatatgccgacctggtgatgct | |
| | cgccagcgaacgccgcgattttgagattgacgaaggttccatttggccatgcctcgaggg | |
| | agttgtcccaacggatttattcattatcaacccagttcgtcctggccagtcatacggcatgtt | |
| | catcaatcgctttaacgagttgatggagcagcgccaatgcgccgcatga | |
| ECOLIN_10290 | atgaccgtatttgaatatctccaggctcatccgaataccaccagcggtgaaatcgccaaag | 196 |
| | gtatgaacaaaaagaccccagcggtcgccggagcattatctcagctctatggcaccggt | |
| | cggatcgtgaagtctggtgttcgcaagggtattccaacataccgcattaacgatatgccgtt | |
| | tggttgcagtaacagcctaaccatgatgtttaaccagctcttgagcagagccagacaagg | |
| | agcagcccaatga | |
| ECOLIN_10295 | atgacagcactcaacaaacaggcgctgcgtgaagaattccagttcatgcaggacaactat | 197 |
| | agcgacccggcagaccacgatcggcaggtgatttacatcgaggcggaggcgctgctg | |
| | gatgagttggaagccaaagactcaacgatagcagcacaacaacatgagatccgtatgtt | |
| | gctgaatgcgcttgaggaaaaaccatgcccgaaatgcaacgacacaggaatgactgata | |
| | gtggcggcacgcagccatggggcgagccgattgagattgaatgcgactgccgacagc | |
| | aggatgccaacaccgcagaacttgtagccgctggcattggcgtgaagggggagtga | |
| ECOLIN_10300 | atggataaattaatcaaacctaccgccaaaggtaaatatgacggttcatgtgattatctttgc | 198 |
| | tcggaagatgcgcgattcatcgttatgcgcggcgattatacggaagcggaaataattcag | |
| | gcttctgtgtctcaagatgtaatcgactcggatggtgcggctgattttgcaagtagcgcccg | |
| | ctattatcagtgctggtacaaagttagcccaataggtggtcaggatggctattcaggctgg | |
| | catcatcctcgtgattcgccgtgtcgcggtgcatatttcgcatcagttttgcaatgggattaa | |
| ECOLIN_10305 | atgacaactaacaaccacccggcgcacggtcctgtatcactcgatcgcctgcaccagata | 199 |
| | cgcgaacacctgctgcatgatacccaatactcaaacggcgggaacagagcctacattct | |
| | cgctgatgtattgaaggtgattgatggggctattgcccgcgagctggtacgccgtgagca | |
| | tgcagcgtggtcacaggctactttcggcgatgtcggtccagttggtccgctgaagcacctt | |
| | tccaaagaagcgctcgaggctgctgctgaaccaggcgaccttagcgaatgggctgacat | |
| | gcaattcctgttatgggatgcgcaacgtcgtgccggtatcagtgatgagcagattacccag | |
| | gcaatgataaaaaagctggctataaataaggttcgccaatggcctgagccgaaagacgg | |
| | ggaacctcgattgcatatcaaagaacagtcagagcaggagaaaaaataa | |
| ECOLIN_10310 | atgtttagcctgattcggcgcggtcaaatctacacggacagtagcaactggcccgtaatta | 200 |
| | tccatagctgtagtgatcactcggtccgaattaaacgcaatgatggcgagctgagaacga | |
| | ttagcatcaaacgctttaacgaagattttgaacgagtggagcatgatgagtatcgcaaaat | |
| | atgtgccgaaatagagcaggaaacaaacctgaaaaacctacgtgcgatgcgtcgcggc | |
| | aagattactgaatag | |
| ECOLIN_10315 | gtgaacaacttaatgatcgaccttgagtccatgggcaaaaaaccgaatgcccctattgtct | 201 |
| | ccattggtgccgtattcttcgatccgcaaagcggtgaactgggtcaggagttttacaccgc | |
| | tgttaatcttgaaagcgctatggagcagggagcggtgccggatggtgacactattctgtg | |
| | gtggttaagacaaagctcagaagcacgatcagcaatctgtgttgatgatgcgatgccgat | |
| | atcatctgccctatctgaactgagccatttcattaatcggcattctgataaccctaaatatttaa | |
| | aagtttggggcaatggagctactttcgacaacgttatattgcgcggcgcatatgagcgtgc | |
| | cggccaggtttgcccgtggcaattttggaatgatcacgacgtcagaaccatcgtcacatta | |
| | ggcagatctgtaggtttcgatcctaagcgtgatatgccatttgatggggttgcacataacgc | |
| | actggctgatgcccgccaccaggcgaaatatgtttcagcgatttggcagaaactaatccc | |
| | aaccaccagcaacagctaa | |
| ECOLIN_10320 | atgagcaatattttccagttagctcccaacgattgggtttgtgaaagcgttttgatcgcggtt | 202 |
| | actgggctcaaacccggaaccatcctccgtgccagaaaagaatgctggatgattgggag | |
| | ggagtatatccacgtatcgcctgacggaaatcctaaaccttccagtgagtgcatgtataac | |
| | agaaaggctgtagatgcctgggtcgcttcaatgaaaagcaagcaaccagggtga | |
| ECOLIN_10325 | atggataaagtcacatatccaacaggcgtcgaaaaccacggtggcacattacgcatctgg | 203 |
| | tttaattttaaaggtaagcgtgtcagggaaagtctcggtgtccctgacaccgctaagaaca | |
| | ggaagatagccggggaactgcggacatcagtatgttttgccatccgcacaggaacctttg | |
| | attatgcaacccagtttcctgactcccctaacctcaaggcttttggtgtaagtaaaaaagac | |
| | attacagtgaaagaacttgaagaaaaatggctggatctgaaacggatggaaatctgcgcg | |
| | aacgcatttaatcgctatgaatctgtcgcaaggaatatggtgccgaggatcggaggtaatc | |
| | gcctggtgtcagcagtaaccaaagaggaattgctgtatctgaggaaatatttgctaactgg | |
| | ttatcagaatccgacgaaaaacaaagccccggcaaaagggcgaagcgttgttactgtga | |
| | actattacatgacgacaatggccggaatgtttcagtttgctgcggatcacggttacttagag | |
| | gtgaacccattcgagggaattaagcctctgaaaaaagccagggcagaaccagatcctct | |
| | gtctcgtgatgaatttattcgcctgatagatgcatgccggcatcagcagacgaaaaacctg | |
| | tggtcattagcagtgtacacaggaatgcgtcacggggaactggtctccctggcctgggaa | |
| | gatatcgacctgaaggctggaacaattaccgtcagacgtaattatacgaaacttggtgagt | |
| | tcactctaccgaaaaccgaggcaagcacagatcgagtggtgcatcttatccagcccgca | |
| | atcagtatcctgaaaaatcaggctgaaatgacaaggctgggcaggcaatatcacattgaa | |
| | gtgcagttacgtgagtacggccgttcggtgaaccatgagtgtacattcgtctttaatccgca | |
| | tgtggtcagacgcagtaagcaggtcggatttatctaccgggtcgattcagtaggcgactc | |
| | atgggaagcggcacttaagcgtgcggggatcagacacagaaaggcgtaccagtcacg | |
| | acacacctatgcgtgctggtcattatcagctggtgcaaaccctagttttattgccagtcagat | |
| | ggggcatgcgagcgcgcagatggtgttcaatgtttacggtgcatggatggctgacagca | |
| | gcgcagagcagatcgcaatgctgaatcagaagctggcagattttgccccattgatgcccc | |
| | atagccacgagaacagtacgggaggattattaaaatcagtaagttaa | |
| ECOLIN_10330 | gtggagggtaacaccacgctttatgccctgccgaaacccgaggttgtcctgcgctggcgt | 204 |
| | gagcagaccacagatgacttccgcttctgttttaagtttccggcgaccatttcgcatcaggc | |
| | agcattacggcattgcgatgatttagtgactgaatttttgacccgcatgtcaccgttggctcc | |
| | gcgcattggacaatactggctgcaactgcctgccacattcggcccacgggagctgcctg | |
| | cgctttggcattttctcgattctcttcccggtgaatttaattatggggtggaagtccgccatcc | |
| | acagtttttcgccaaaggggaagaggaacaaacgcttaatcgcggtttacatcagcgcgg | |
| | cgttaatcgggtgattttagacagccgcccggttcatgcagcacgtccatacagtgaagct | |
| | attcgcgacgctcaacgaaaaaaacctaaagttccggtacatgctgtactgacggcgaaa | |
| | aatccactgatccgttttatcggtagtgatgatatgacgcaaaaccgggaattatttcaggtc | |
| | tggttacaaaaattagcgcagtggcatcagaccactacgccttatctttttttacatacgcca | |
| | gatattgcccaggccccggaactggtacataccctgtgggaagacttacgtaaaacgctt | |
| | ccagagatcggagcagttccggctattccacagcaatcttctcttttctga | |
| ECOLIN_10335 | atggtaagcgcgctgtatgccgttttaagtgcgttgttattaatgaagttctcttttgatgtcgtt | 205 |
| | cgcctgcgaatgcagtaccgcgttgcctatggcgacggcggttttagcgaactgcaaag | |
| | cgctattcgcattcatggtaacgcggtggaatatattcctatcgcgattgtgttgatgctgttt | |
| | atggaaatgaatggcgcagaaacctggatggtgcatatttgcggcatcgttttgcttgctg | |
| | gtcgtctgatgcattattacggttttcatcaccgtctgttccgctggcgacgttctggcatga | |
| | gcgccacctggtgtgcgctgttgctgatggtgctggcgaatctttggtatatgccctggga | |
| | gttggttttctccctgcgttag | |
| ECOLIN_10340 | atgtctcaccgcgacacgctattttctgcccctatcgccagactgggcgactggacctttg | 206 |
| | atgaacgggtagctgaagtcttcccggatatgatccagcgttccgttcccggctattccaat | |
| | attatttccatgattggtatgttagccgagcgcttcgttcaacctggtacgcaggtttacgatc | |
| | tgggttgttctctgggcgcggcgacgctctcggtgcgtcgcaacattcatcatgataattgc | |
| | aaaattattgccatcgacaactccccggcgatgattgaacgctgccgtcgtcatattgacg | |
| | cctataaagcccctacgccagtagacgttattgaaggtgatattcgcgatatcgccattgaa | |
| | aacgcatcgatggtggtgctgaattttaccctgcaattcctggaaccttccgagcgccagg | |
| | cgttactggataaaatttatcaagggctgaacccgggcggtgcgctggtgctttcggaaa | |
| | aattcagtttcgaagatgccaaagttggtgaactgctgttcaacatgcaccacgactttaaa | |
| | cgtgccaacggttacagcgaactggagatcagccagaaacgcagcatgctggaaaacg | |
| | tgatgctgaccgattccgtggaaacccataaagcacgcctgcataaagccggttttgagc | |
| | atagcgagctgtggttccagtgctttaactttggttcactggtggcattaaaagcagaggac | |
| | gctgcatga | |
| ECOLIN_10345 | atgatcgactttggtaacttttattctctgattgccaaaaatcatctttcacactggctcgaaac | 207 |
| | gctgcccgcgcagattgctaactggcagcgcgagcagcagcacgggctgtttaagcag | |
| | tggtccaacgcggtggaatttctgcctgaaattaaaccgtatcgtctggatttattgcatagc | |
| | gtaaccgccgaaagcgaagagccactgagcgccgggcaaattaagcgcattgaaacg | |
| | ctgatgcgcaacctgatgccgtggcgcaaagggccgttctcactgtatggcgtcaacatc | |
| | gataccgaatggcgttccgactggaaatgggatcgcgttatgccccatctttctgatttaac | |
| | cgggcgcaccattcttgatgtcggctgtggcagcggttatcacatgtggcgcatgattggc | |
| | gcaggggcgcatctggcggtgggtatcgatcccacgcagctattcctctgccagtttgaa | |
| | gcagtgcgtaaactgctgggtaacgatcagcgcgcacatttgttaccgttaggtattgaac | |
| | aacttccggcactgaaagcctttgataccgtcttttcgatgggcgtgctttatcatcgtcgttc | |
| | accgctggagcatctctggcagttaaaagaccaactggtgaatgaaggcgaactggtgc | |
| | tggaaacgctggttattgatggcgacgaaaacacggtgctggtgccgggcgatcgttac | |
| | gctcaaatgcgtaatgtctatttcattccttccgcgctggcgctgaaaaactggctgaagaa | |
| | gtgtggttttgttgatattcgcattgcagatgtgagcgttaccaccacagaagagcagcga | |
| | cgcaccgaatggatggtcaccgagtctctggccgattttctcgacccgcatgatccgggt | |
| | aaaacggtggaaggttatcctgcgcctaaacgcgcggtgctgattgcgcgcaagccgta | |
| | a | |

TABLE G
Polypeptide sequences encoded by the genome of E. coli Nissle Phage 3.

| | | |
|---|---|---|
| ECOLIN_ | METKKNNSEYIPEFDKSFRHPRYWGAWLGVAAMAGIALTPPKFRD | 208 |
| 09965 | PILARLGRFAGRLGKSSRRRALINLSLCFPERSEAEREAIVDEMFAT | |
| | APQAMVMMAELAIRGPEKIQPRVDWQGLEIIEEIRRNNEKVIFLVP | |
| | HGWAVDIPAMLMASQGQKMAAMFHNQGNPVFDYVWNTVRRRF | |
| | GGRLHARNDGIKPFIQSVRQGYWGYYLPDQDHGPEHSEFVDFFAT | |
| | YKATLPAIGRLMKVCRARVVPLFPIYDGKTHRLTIQVRPPMDDLLE | |
| | ADDHTIARRMNEEVEIFVGPRPEQYTWILKLLKTRKPGEIQPYKRK | |
| | DLYPIK* | |
| ECOLIN_ | MQQIARSVALAFNNLPRPHRVMLGSLTVLTLAVAVWRPYVYHRD | 209 |
| 09970 | ATPIVKTIELEQNEIRSLLPEASEPIDQAAQEDEAIPQDELDDKIAGE | |
| | AGVHEYVVSTGDTLSSILNQYGIDMGDITQLAAADKELRNLKIGQ | |
| | QLSWTLTADGELQRLTWEVSRRETRTYDRTAANGFKMTSEMQQG | |
| | EWVNNLLKGTVGGSFVASARNAGLTSAEVSAVIKAMQWQMDFR | |
| | KLKKGDEFAVLMSREMLDGKREQSQLLGVRLRSEGKDYYAIRAE | |
| | DGKFYDRNGTGLAKGFLRFPTAKQFRISSNFNPRRTNPVTGRVAPH | |
| | RGVDFAMPQGTPVLSVGDGEVVVAKRSGAAGYYVAIRHGRSYTT | |
| | RYMHLRKILVKPGQKVKRGDRIALSGNTGRSTGPHLHYEVWINQQ | |
| | AVNPLTAKLPRTEGLTGSDRREFLAQAKEIVPQLRFD* | |
| ECOLIN_ | MLHKKTLLFAALSAALWGGATQAADAAVVASLKPVGFIASAIAD | 210 |
| 09975 | GVTETEVLLPDGASEHDYSLRPSDVKRLQNADLVVWVGPEMEAF | |
| | MQKPVSKLPEAKQVTIAQLENVKPLLMKSIHGDDDDHDHAEKSDE | |
| | DHHHGDFNMHLWLSPEIARATAVAIHGKLVELMPQSRAKLDANL | |
| | KDFEAQLASTEKQVGNELAPLKGKGYFVFHDAYGYFEKQFGLTPL | |
| | GHFTVNPEIQPGAQRLHEIRTQLVEQKATCVFAEPQFRPAVVESVA | |
| | RGTSVRMGTLDPLGTNIKLGKTSYSEFLNQLANQYASCLKGD* | |
| ECOLIN_ | MTSLVSLENVSVSFGQRRVLSDVSLELKPGKILTLLGPNGAGKSTL | 211 |
| 09980 | VRVVLGLVTPDEGVIKRNGKLRIGYVPQKLYLDTTLPLTVNRFLRL | |
| | RPGTHKEDILPALKRVQAGHLINAPMQKLSGGETQRVLLARALLN | |
| | RPQLLVLDEPTQGVDVNGQVALYDLIDQLRRELDCGVLMVSHDL | |
| | HLVMAKTDEVLCLNHHICCSGTPEVVSLHPEFISMFGPRGAEQLGI | |
| | YRHHHNHRHDLQGRIVLRRGNDRS* | |
| ECOLIN_ | MIELLFPGWLAGIMLACAAGPLGSFVVWRRMSYFGDTLAHASLLG | 212 |
| 09985 | VAFGLLLDVNPFYAVIAVTLLLAGGLVWLEKRPQLAIDTLLGIMA | |
| | HSALSLGLVVVSLMSNIRVDLMAYLFGDLLAVTPEDLISIAIGVVIV | |
| | VAILFWQWRNLLSMTISPDLAFVDGVKLQRVKLLLMLVTALTIGV | |
| | AMKFVGALIITSLLIIPAATARRFARTPEQMAGVAVLVGMVAVTG | |
| | GLTFSAFYDTPAGPSVVLCAALLFIISMMKKQAS* | |
| ECOLIN_ | MIEADRLISAGTTLPEDVADRAIRPKLLEEYVGQPQVRSQMEIFIKA | 213 |
| 09990 | AKLRGDALDHLLIFGPPGLGKTTLANIVANEMGVNLRTTSGPVLE | |
| | KAGDLAAMLTNLEPHDVLFIDEIHRLSPVVEEVLYPAMEDYQLDI | |
| | MIGEGPAARSIKIDLPPFTLIGATTRAGSLTSPLRDRFGIVQRLEFYQ | |
| | VPDLQYIVSRSARFMGLEMSDDGALEVARRARGTPRIANRLLRRV | |
| | RDFAEVKHDGTISADIAAQALDMLNVDAEGFDYMDRKLLLAVID | |
| | KFFGGPVGLDNLAAAIGEERETIEDVLEPYLIQQGFLQRTPRGRMA | |
| | TVRAWNHFGITPPEMP* | |
| ECOLIN_ | MIGRLRGIIIEKQPPLVLIEVGGVGYEVHMPMTCFYELPEAGQEAIV | 214 |
| 09995 | FTHFVVREDAQLLYGFNNKQERTLFKELIKTNGVGPKLALAILSGM | |
| | SAQQFVNAVEREEVGALVKLPGIGKKTAERLIVEMKDRFKGLHGD | |
| | LFTPAADLVLTSPASPATDDAEQEAVAALVALGYKPQEASRMVSK | |
| | IARPDASSETLIREALRAAL* | |
| ECOLIN_ | MNINYPAEYEIGDIVFTCISAALFGQISAASNCWSNHVGIIIGHNGE | 215 |
| 10000 | DFLVAESRVPLSTITTLSRFIKRSANQRYAIKRLDAGLTEQQNQRIV | |
| | EQVPSRLRKIYHTGFKYESSRQFCSKFVFDIYKEALCIPVGEIETFGE | |
| | LLNSNPNAKLTFWKFWFLGSIPWERKTVTPASLWHHPGLVLIHAV | |
| | GVETPQPELTEAV* | |
| ECOLIN_ | MAIILGIDPGSRVTGYGVIRQVGRQLSYLGSGCIRTKVDDLPSRLKL | 216 |
| 10005 | IYAGVTEIITQFQPDYFAIEQVFMAKNADSALKLGQARGVAIVAAV | |
| | NQELPVFEYAARQVKQTVVGIGSAEKSQVQHMVRTLLKLPANPQ | |
| | ADAADALAIAITHCHVSQNAMQMSESRLNLARGRLR* | |
| ECOLIN_ | MAGHSKWANTRHRKAAQDAKRGKIFTKIIRELVTAAKLGGGDPD | 217 |
| 10010 | ANPRLRAAIDKALSNNMTRDTLNRAIARGVGGDDDANMETIIYEG | |
| | YGPGGTAIMIECLSDNRNRTVAEVRHAFSKCGGNLGTDGSVAYLF | |
| | SKKGVISFEKGDEDTIMEAALEAGAEDVVTYDDGAIDVYTAWEE | |
| | MGKVRDALEAAGLKADSAEVSMIPSTKADMDAETAPKLMRLIDM | |
| | LEDCDDVQEVYHNGEISDEVAATL* | |
| ECOLIN_ | MAYKRPVSILVVIYAQDTKRVLMLQRRDDPDFWQSVTGSVEEGET | 218 |
| 10015 | APQAAMREVKEEVTIDVVAEQLTLIDCQRTVEFEIFSHLRHRYAPG | |
| | VTRNTESWFCLALPHERQIVFTEHLAYKWLDASAAAALTKSWSNR | |
| | QAIEQFVINAA* | |
| ECOLIN_ | MRTEYCGQLRLSHVGQQVTLCGWVNRRRDLGSLIFIDMRDREGIV | 219 |
| 10020 | QVFFDPDRADALKLASELRNEFCIQVTGTVRARDEKNINRDMATG | |
| | EIEVLASSLTIINRADVLPLDSNHVNTEEARLKYRYLDLRRPEMAQ | |
| | RLKTRAKITSLVRRFMDDHGFLDIETPMLTKATPEGARDYLVPSRV | |
| | HKGKFYALPQSPQLFKQLLMMSGFDRYYQIVKCFRDEDLRADRQP | |
| | EFTQIDVETSFMTAPQVREVMEALVRHLWLEVKGVDLGDFPVMT | |
| | FAEAERRYGSDKPDLRNPMELTDVADLLKSVEFAVFAGPANDPKG | |
| | RVAALRVPGGASLTRKQIDEYGNFVKIYGAKGLAYIKVNERAKGL | |
| | EGINSPVAKFLNAEIIEAILERTGAQDGDMIFFGADNKKIVADAMG | |
| | ALRLKVGKDLGLTDESKWAPLWVIDFPMFEDDGEGGLTAMHHPF | |
| | TSPKDMTAAELKAAPENAVANAYDMVINGYEVGGGSVRIHNGD | |
| | MQQTVFGILGINEEEQREKFGFLLDALKYGTPPHAGLAFGLDRLT | |
| | MLLTGTDNIRDVIAFPKTTAAACLMTEAPSFANPTALAELSIQVVK | |
| | KAENN* | |
| ECOLIN_ | MLELNAKTTALVVIDLQEGILPFAGGPHTADEVVNRAGKLAAKFR | 220 |
| 10025 | ASGQPVFLVRVGWSADYAEALKQPVDAPSPAKVLPENWWQHPA | |
| | ALGATDSDIEIIKRQWGAFYGTDLELQLRRRGIDTIVLCGISTNIGVE | |
| | STARNAWELGFNLVIAEDACSAASAEQHNNSINHIYPRIARVRSVE | |
| | EILNAL* | |
| ECOLIN_ | MGFPSPAADYVETRISLDQQLISQPAATYFMRASRSHFREGIIQGAL | 221 |
| 10030 | LVVDASLTACDGSLLICAIDGEFRIKRYRTHPQPHLVNLDNGRREA | |
| | LPADDDGYSSAPAIFGVITYIINDARNAEFDDCPVM* | |
| ECOLIN_ | MFVELVYDKRNFDGLPGAKDIILGELTKRVHRIFPDADVRVKPMM | 222 |
| 10035 | TLPAINTDASKHEKEQISRTVQEMFEEAEFWLVSE* | |
| ECOLIN_ | MLWRIFIFVNVGLGEAAKRNVGTGENQIPDMSAFPSGNNWFQLPS | 223 |
| 10040 | GHIVQIFSMNVLGADANGTSANYPIAFPTTMIAVSALWSDGTVAN | |
| | APTYKMMGNTTNRTTLTIKVSASSGTYGTMIIAVGR* | |
| ECOLIN_ | MNKYSYSPSENAFYAVALKNTYELSGTWPADALDIPDDISVKYMA | 224 |
| 10045 | EPPQGKIRVAGENGFPTWAEIPPPSHEELIEQAESERQLLINQANEY | |
| | MNSKQWPGKAAIGRLKGEELAQYNSWLDYLDALELVDTSGTPDIE | |
| | WPTPPAVQAR* | |
| ECOLIN_ | MKPVFDENGLAAVPGDMRCFYYDAVTSEYTGWSDEYINTGVSMP | 225 |
| 10050 | ACSTGIDPDENIPGRVAVFTGKGWSHEEDHRNETVYSIENGAAVT | |
| | VDYIGAIKNGYVTLSPLTPYDKWDGEKWVTDTEAQHGASVEAAE | |
| | AQRQSLIDAAMASISLIQLKLQAGRKLTQAETTRLNAVLDYIDAVT | |
| | ATDTSTAPDVIWPELPEA* | |
| ECOLIN_ | MIYSTGTISINGNTATGSGTNWTAPASQVRAGQTIIVMSNPVQLFQI | 226 |
| 10055 | SSVNSATSMTVTPAVSPALSGQKYGILVSDNISVDGLAQAMSQLIK | |
| | EYDENIGAWETFATTSANQSITVTINGTAVTIPGIGKLAQKGSNGA | |
| | VTVADGGTGATNAADARTNLGLGEGSPAIGVPFFWPSAAMPNTVI | |
| | DSWSSMVFLKFNGAKFSATDYPVLAKVFPSLVLPEARGDFIRVWD | |
| | DGRGADGGRELLSWQEATNFSQFAGNIGGGAGHAINFHDGIAGNQ | |
| | PGFSRFNFNSNSVGDGVNFVAVRPRNIAFNFLVRAK* | |
| ECOLIN_ | MGEKMQLLKQETILQGAKGGGGSSHTPVEQPDDLLSVAKLKMLIA | 227 |
| 10065 | VSEGEIQGDLTAQNIFLNDTPLANDSGEYNFSGVKWEFRKGTQDQ | |
| | TYIAGMPQVDNELAVGTTVTTTAPWTRQFTNLSLDAIRIKLSLPVQ | |
| | YLYKDNGDMVGTVTEYAIDLSTDGGAWKTVVNGKFDGKTTTEY | |
| | QRDHRIDLPKSTSGWSVRVRRITADASGSNSKLVNAFKVFSYAEVI | |
| | DSKLRYPLTALLYVEVDSSQFNGSAPKVTCKIKGKLIKVPDNYDPK | |
| | TRTYSGSWSGGFKMAWSNNPAWIFYDLVLDEIYGMGTRVDASMV | |
| | DKWALYSIAQYCDEMVSDGAGGTEPRFTCNVFIQSQEDAWQVLN | |
| | DLAAVFRGITFWGNDQIYVQADVPQDDVDWVYNVSNVIDGLFTY | |
| | AGGSYKNRYSSCLVSWSDPQNHYSDTVEGVYDSALVERYDVRQT | |
| | SLTAIGCTSQSEAHRRGRWVLLSNAKDGTVSFGVGLDGYIPLPAEII | |
| | GVADPFRSGKENGGRISAANGRQITLDREIDYAAKDRLVVNLPDG | |
| | KAQTRTISAVSADKKTVTVATAFSQVPVAGAVWAIDSDNLAIQYF | |
| | RVTSIAANDDSTGGFTITAVQHDPNKYRYIDDGVRVESPPITVTPIS | |
| | VLSAPKNIVVTESDHVSQGLTVASLDVSWDKVEGAIRYVAQWRK | |
| | DNGDWINVPVTSAQGFSVQGIYSGSYDVRVRALNAQDTSSPWGY | |
| | GETTYLSGKTGKPGTPLNFLATEDVVWHIDLTWKFPDGSGDTAYT | |
| | EIQRATTADYANPELLVLVPYPAADYQHGPMPAGVRQWYRARLI | |
| | DRIGNAGDWTDWIMGTSSIDVSEITNDILEDMKESETFKDLIENAV | |
| | DSNEKIAGMANDIKQANDELEQQAKDIAKNAQDVGKVQTSVNEL | |
| | SSTVGNVSSSLSQLEQTVATADTALGQRIDNISVSMDGMTGGVKN | |
| | SAIAIIQANLAQVATRKTLSASVAGNSANLDRLDEVIVSEKEATAR | |
| | SLLSLQTDVNGNKASINSLNQTLSDYQQATATQINGITATVNGHTS | |
| | AITTNAQAIANVNGELSAMYNIKVGVSSNGQYYAAGMGIGVENTP | |
| | SGMQSQVIFLADRFAVTTAAGNSVALPFVIQNGQTFIRASFIQDGTI | |
| | ENAKIGNYIQSNNYAAGSAGWKLNKAGDAEFNNVTVRGVVYASG | |
| | GSFTGEIQATSGKFKGTVEAQSFIGDIANMHTGTNVSRSSDGYLEK | |
| | VMTYNDSSSSGHARHVCVIANVKGNGAGTIDINGNESGSSVQDME | |
| | RLIMHSAVVTGPNVTVRIKVSAQNNRGASISSPTIIVSHGSGSFTG* | |
| ECOLIN_ | MVKTLILEGKMAKKFGKRVQFDVADLREMLRAMCSQVPGFKKY | 228 |
| 10070 | MSEAHMKGIRFAFFNGGNNIGLEEFDMTRGGSVYRIVPVYEGAKS | |
| | SGVLQIVVGAVALVAAFFTAGASMAAWGAAMSATAISATSILTGV | |
| | GVSMMLGGVVQMLTPQPSFGAGKSSSTDNTPNYAFGAPVNTVAM | |
| | GHPVPLAYGLTEAGGAIVSAGMYSSDQQ* | |
| ECOLIN_ | MREKLLDAIRQHVAAEYPKEACGLIVQSGQQQIFIPCRNIADKPEET | 229 |
| 10075 | FTLSPEDQLAARARGEIIMLIHSHPDVVRLVPSELDRIQCDWSGIEW | |
| | GIMSWPDGDFCTISPREDRDYAGRQWVLGYADCWSLIREFYLREY | |
| | GIVLGNYSVPYEWWESGKERLYDDNWEREGFVEIAAGAMQPGDII | |
| | MMSVQASVTNHAAVYVGDNIILHHLFGHLSSRTPYGKYYRDRTV | |
| | RVVRHKDRMHG* | |
| ECOLIN_ | MSFTADIQQLEPGSVIQLIEIDGTEFGMDQVLRFHAHNIQEEGWAA | 230 |
| 10080 | FAAENLPAIIWQGNQYDPHPYELKGMELSSTGSQPTPTLSVGNVGN | |
| | YVTALCLEYDDMVRAKVKIHTTLSKYLDAANWKNGNPGASPADE | |
| | RLQLFYVNAKTAETRVQVDFELCSPFDIQSLQLPTRQITPVCTWCM | |
| | RGWYRSGTGCDYNGTKYFTKDGTPTDDPSKDVCGGRRQDCQDR | |
| | HGPDAPLPFGGFPAANLQGK* | |
| ECOLIN_ | MTDTFTWRTRKTAQGTETARTLQAQFGDGYKQIAGMGINDKQET | 231 |
| 10085 | WNLDWTGTRQEAAALRAFLMSHVTKSFWWTTPWGEKKLFRMK | |
| | ADSFSVSFPTGKKATVAFTFEQAFAP* | |
| ECOLIN_ | MAQQISDLVINLDVDSATFSEQVARIKGQLTGMAEDSEKVQTRMQ | 232 |
| 10090 | RASERQAAAFKTVGDAGAAAAADMKSRQSAATEGLTKDWQNVS | |
| | KSVDETHRRVTELNQRMRENDGQAAALARRQDELAASFFRQIDG | |
| | VRQLNGETQSLANVQARFRAARAQGNITQQDYLALISRTTARQKE | |
| | LQIVEEKSAAARTRFLSQLKQQVAEQKLSGTELLRMKAAQVGASD | |
| | AAEVYIRKLEAAKVATHGLGLQSAAARQELGILIGEVMRGNFGAL | |
| | RGSGITLANRAGWIDQLLSLRGLGIASMVGGIAAAVFGLGKAWYD | |
| | GSKESEEFNRQLILTGNYAGKTSGQLQALARSLAGNGITQHAAAG | |
| | VLAQVVGSGAFSGNDVSMVSNVAARLQQATGQAVDETINQFKRL | |
| | KDDPVNAVATLNDSLHFLTATQYEQIASAQALGDSQKAAELAMR | |
| | AYSDAVIQRAGAVEDNLGSLEKAWNWVKNAASGAWDAMLGVG | |
| | RNPDTAMKRQDSFAEWQAAEKEYRALSSNLKVDPDYAGNNVLQ | |
| | KADAERLRNARQQVELKKQAYDLADQQYAQEGLAAAREKMRTD | |
| | QQAQAIRSQQQFNQLVESGATAAEKRASAEKKLSQLIEKNRQDAK | |
| | DGVATLWTEKDIAAARAGIEKQWKDPKTPKGKSYSTPAGDKAEE | |
| | KAQAELLTLQAQLKTLEQHTSVNDVISKQRQDLWQTENQFTVLQE | |
| | AAGRRQLTAQEKSLLAHKEETLEYKRQLADLGDKVASQQKLNQL | |
| | ADQAVKFEQQQKAARAGLQAQSEGLSTREAGRQTTLQRLSESYSY | |
| | NPQAQQKVLEEQRATFEAEDALRANWLAGAKQGWAEYQDSATN | |
| | VFSSVQQISQATFSGLAGQLTSLTTTGKASFREFTSSILKMIVSVINQ | |
| | LLVAYTIQSAMGWVSGGAKTSSAGQSFAVPSYRPQGFDVGGFTGH | |
| | GGKYEPAGIVHRGEFVFTKESTSRIGVANLYRLMRGYASGGLVGG | |
| | GNAAGAGMGGISVYAPVSISQQGSDGSINQANATGTAKQLQAIVQ | |
| | QTITERLKKEMSAGGVLYSRRTQ* | |
| ECOLIN_ | MLAGMTSTELGDWHQFYREHYFQDAQLDAHFSELLYSISTLFFRD | 233 |
| 10095 | PELTPAHFSLLSPSGIVISDDEPDDDALMAAAEGITGGIRYGPAD* | |
| ECOLIN_ | MFLKKEKFTWQKESLTIFELSALQRIEYITFMAAEEKAVSADSDGIS | 234 |
| 10100 | DQEMTARLIGSNIRCGARLIAMSLWHNDPAGTDVETLYQQVLSG | |
| | WPPEAIGKAEMEIKLLSGMLVPVEDDKAADPDAPAEAESAEPVAA | |
| | EKPLPAS* | |
| ECOLIN_ | MPTPNPLAPVKGAGTTLWLYTGTGNAFANPLSDIDWNRLAKIKEL | 235 |
| 10105 | TPGEMTAESYDDTYLDDEDADWNATAQGAKSAGDTSFTLAWKP | |
| | GEEGQKDLVAWFIDGSVRYYKIKYPNGTVDVFRGWCSSLGKAIPA | |
| | KEVITRTAKITNTGKPELAEESGTPNIPVTGVTLDKATASVAVGATT | |
| | TLNVTVNPASASDTSFRVATSDGAKATVTVSGNAITVTGVAAGTA | |
| | DVIVMTSDGNFVAVCKVTVTAA* | |
| ECOLIN_ | MNRHSAIRAAILAKLKAEITDTVTWFDGRPVFLEEQDLPAVAVYLS | 236 |
| 10110 | DAEYTGDSLDEDSWQAVVHIEVFLKASSPDSALDSWMEEKVYPA | |
| | MAFIPGLTELVETFTPQGYDYQRDDEMATWGSVDFTYLITYSI* | |
| ECOLIN_ | MKGLERAIQNLNSLSRLIVPEATAKALNRVARRTISQGSKAVAKEA | 237 |
| 10115 | TVDDNRKKGLPVRLVRQRSRLRKARHDRPVASIKINRGNLPAIKLG | |
| | TARVRLSRKKGARNGAGSVLKIGPYTFRNAFIQQLANGRWQVMR | |
| | RVGQARYPIDVVKVPLETPLTVAFTAISKRLIESDMPKELSAALKN | |
| | QLRIHLKR* | |
| ECOLIN_ | MDAATIKKMGKTAIINGSSYDVVPAEQLEEMGPLSGTGTSLVVFSE | 238 |
| 10120 | LYQPRRNDSVDYDGKNLIVTRYDMFNGKPRIHLE* | |
| ECOLIN_ | MAKNYAQDGKTIPLVNSGATDVHSGDPVVVGKLIAVAITDIPAGD | 239 |
| 10125 | TGDGFTEGVFLLPKVSADAVTAGAQVYLKDGKITIEETDAVAAGI | |
| | AWEDAGANTTVVEVKINA* | |
| ECOLIN_ | MQASNNSEADIFIYDEIGYWGVTAKQFVNDLRALGDVTHINLYINS | 240 |
| 10130 | PGGDVFDGIAIYNALKHHGAAITVHIDGLAASMASVIAMVGNPVI | |
| | MPENTMMMIHKPWGFAGGDASDMRDYADLLDKVESVLIPAYAQ | |
| | KTGKSTEEIAVMLEDETWMNGSECLELGFADQVTPSLQAMACIHS | |
| | KRIEEFEKMPKSIRNMITPPRNTTQRDPVITQPQAPQAKTDPAPDEN | |
| | AIRAQVLAEQKTRVNAIGDLFAMFGNKHMELQNQCVADPDCSVD | |
| | KAKDLLLAELGKTATPSNKTTQPHIHAGNGNFVADGIRQALMARA | |
| | GFEGQERDNVYNGMTLREYARMALTEKGIGVASYNPMQMVGLA | |
| | LTHSTSDFGNILLDVANKALIQGWDEAQETFEQWTKKGQLSDFKT | |
| | AHRVGMGGFPSLRQVREGAEYKYITTSDKGETIALATYGEIFSVTR | |
| | QAIINDDLNQLTDVPMKMGRAAKATIGDLVYAILTKNPKLSDGKA | |
| | LFHADHKNLSSGAISVASLDESRKLMRLQKEGERTLNIRPAYMLVP | |
| | VALETLANQTIKSASVKGADINAGIVNPIQNFAEVIAEPRLDEADAK | |
| | AWYLAAAKGTDTIEVAYLNGVDTPYIDQQEGFTTDGIATKVRIDA | |
| | GVAPLDYRGMTKSSGQ* | |
| ECOLIN_ | MSFLDDAIGLFSPGWKASRLRARAVIKAYEAVKQTRTHKAQKENR | 241 |
| 10135 | SADQLSQMGAVSLRQQARWLDNNHDLVIGVFDKLEERVVGAKGI | |
| | IVEPHPMLSNGKIAKKLATDIRRKWGEWSVRPDVTTQFTRPMLER | |
| | LMLRTWLRDGEVFAQLVRGTGNGLQPVAGVPFWLEALEPDFVPM | |
| | NSDAATQLNQGVFVDNWGRPKKYQVYKSLPVSGRQFDTKEIDAE | |
| | NMLHLKFTRRLHQTRGTSLLSGVLMRLSALKEYEDSELTAARIAA | |
| | ALGMYIKKGDGQSFDSDSSSDDRELMIQPGMLYDELQAGEEIGMI | |
| | KSDRPNPNLESFRNGQLRAVSAGSRLSFSSTSRNYNGTYSAQRQEL | |
| | VESTDGYLILQDWFIGSVTRPMYRAWLKMAIAAGEIKLPRGIDMD | |
| | SLYNAVYSGPVMPWIDPVKEANAWKTQIRGGAATESDWIRASGR | |
| | NPDDVKSRRKAEVDENREQGLVFDTDPANDKG6TSAEAKEPGAP | |
| | PSESQRKK* | |
| ECOLIN_ | MNPADIQNMIDRYAAAELSVLEGKSITFNGQQMTLENLSEIRKGRQ | 242 |
| 10140 | EWERRLATLNNKRRGRPGYRLARFG* | |
| ECOLIN_ | MAKRASARDIRRDVSGILRAPRRMPVADAVSTYMRVPMGAGNSV | 243 |
| 10145 | PWDPDLAPYVIEPMNCLASREYDAVVFVGPARTGKTIGLIDGWIV | |
| | YNIVCDPADMLVIQVSEEKAREHSKKRLDRTFRCSPEVKTRLSPRR | |
| | NDNNVYDRTFRAGNYLKLGWPSVNIMSSSDYKSVALTDYDRFPE | |
| | DIDGEGDAFSLASKRTTTFMSSGMTLVESSPGRDIRDTKWRRSTPH | |
| | EAPPTTGILSLYNRGDRRRLYWPCPHCGEYFQPEMDNMTGYRDSS | |
| | DPVLASEAAFLQCPACKGRITPDMKRALNMKCVWLRDGQTIDRK | |
| | GLVSGDGRRSRIASFWMEGPAAAYQTWAQLIYKFLTAEQEYESTR | |
| | SEETLKTVINTDFGRPYLPRASMEQRKSELLEQRAEDVPKRSVPNG | |
| | VQFLTATVDVQAGRNRRFVVQITGYGSMGERWIVDRYNIRHSLRC | |
| | DGNGESIQVDPASYPEDWDLLLTDVFDKTWPLAADPSKGMRLMS | |
| | MAVDSGGEDGVTDNAYKFWRRCRREGLGKRIYLFKGDSVRRSKL | |
| | IQRTFPDNTGRSTRRAQATGDVPLYLLQTDALKDRVNNALWRDSP | |
| | GPGYVHFPAWLGSWFYDELTYEERSNEGKWSKPGRGANEAFDLL | |
| | VYADALAILSGYEKIKWPSAPEWARRETWIEDTQTEAGEMPSPPPA | |
| | PKSKPKPKREKPVTEQANPWSSSGGWV* | |
| ECOLIN_ | MDQEIATLKLNINQLAGITGVHRQTVAARLKNVEPAPGSNSKLKL | 244 |
| 10150 | YLVTDILTELMIPTVSANIDDMPPSDRLSHWKAENERLKFEQDTGQ | |
| | LIPADEVAREFSLMAKAVVMVLETLPDVLERDCALTPAAVVRVQS | |
| | VIDDLRDQMAEKVQDAGKEEEQPEED* | |
| ECOLIN_ | MSNKIITLSGAANEVLYALFFRGALLSGDLPSKSGTAELRELGFAET | 245 |
| 10160 | RHTATEYQKENHFTFLTSEGQKFAVEHLVNTRFGEQQYCASMTLG | |
| | VEIDTSAAQKAIDELDQRIRDTVSFELIRNGVSFIKDAAIANGAIHA | |
| | AAIETPQPVTNIYNISLGIQRDEPAQNKVTVSADKFKVKPGVDTNIE | |
| | TLIENALKNAAECAALDVTKQMAADKKAMDELASYVRTAIMMEC | |
| | FPGGVIWQQCRR* | |
| ECOLIN_ | MKILAFINPPGSYMQIETKEMVIFLRDDGISLQYPDGQCAGYGIDG | 246 |
| 10165 | NMLIFMGQSCELFPTKIILHDQRTTNFTHK* | |
| ECOLIN_ | MDYYHEIDFPSLFARAVESDDDVGTTLRIHLLCERMVEAWICACC | 247 |
| 10170 | DCQDLFGRDKNKLLIECNTKISMAGNLGIPPELMKSLKTINSMRND | |
| | LAHNPSIQSIADSRIQSLKDTLTEYFKQHPTEPSMEESKLGIFNAENQ | |
| | LTEEVSLDSDSSKNRLKLILLFSKLMQALMQLVAANHNGRWDNQF | |
| | SQFVYHVTMNATKR* | |
| ECOLIN_ | MTVVLTAKQIEDLAAFAKEDGQPQYTITTGTIPEFEADNGEIIPGYT | 248 |
| 10175 | GLIAYSESLDHGVLQLDD* | |
| ECOLIN_ | MVIVALVGSFLAGSEWTNRSWKIKWADRDSAESSQEANAQTAAR | 249 |
| 10180 | MIEQGRTIARDEAVKDAQAQAASAAVTSAGLATTVKQLRAEATK | |
| | LATHMDAAKHTADLATSVRSKTAGANAAMLADMLGSLAEAARY | |
| | YAGRSDESYRAGMTCERIYESVRLSNNQ* | |
| ECOLIN_ | MAMSLKLKNKLSAAVVGLILAGASAPVILDQFLDEKEGNSLTAYR | 250 |
| 10185 | DGGGLWTICRGATMVDGKPVVQGMKLSAEKCAQVNAIERDKAL | |
| | AWVDRNIKVPLTEPQKAGIASFCPYNIGPGKCFPSTFYKRINAGDT | |
| | KGACEAIRWWIKDGGRDCRLTKGQKDGCYGQVERRDQESALTC | |
| | WGIDQ* | |
| ECOLIN_ | MTRMSTIYSRLSYGSGTTLAGCGVSAKAYAETAKTAKEVSWMLA | 251 |
| 10190 | DRIAGLSLSDWAIIVGIACTVITCAVNWYFRWKEREDRRNGYVSK | |
| | AEE* | |
| ECOLIN_ | MKNTVKINSVELINADSLHYVATLPDNSIDLIVTDPPYFKVKPNGW | 252 |
| 10195 | DNQWKGDEDYLRWLDSCLAEYARVLKPAGSIYLFCGHRLASDIEI | |
| | MMRARFNVLNHIIWAKPSGRWNGCNKESLRAYFPSTERILFAEHY | |
| | LGPYTGKEDVYERKSTELKQHIMTPLIDYFRNARESLGVSSKEIAE | |
| | ATGKKNMASHWFGASQWQLPNEVDYRKLQELFTRIAIDKHIQQK | |
| | LEHPHHQLVATYQSLNRKYSELLEEYKILRRCFSVSALVPYTDVW | |
| | THKPVQFYPGKHPCEKPADMLKQIISASSRPGDIVADFFMGSGSTV | |
| | KAAIELGRRAIGVELEADRFIQTTEEVEKLKKS* | |
| ECOLIN_ | MLNQEDMTETAKAVFNELSDKPATAGEIAQNTHLSHERCQLILTQ | 253 |
| 10200 | LVMAGLSDYQFGCYKRLQ* | |
| ECOLIN_ | MPNWIDVLGEMGTIAQRTPADEVRHKYLRDLSKHTGRNVISYYSG | 254 |
| 10205 | FLQKGGPGFQHLIQMSDDDKNGLMSAINGLDTSLGLDILLHTPGG | |
| | DIAALESIGHYLRSKFGTNIRAIVPMISMSCGTMLACCAEQIVLGKQ | |
| | SNLGPIDPQFNGLSSHAIIEEYERAKAEIFANPAALQWWQFTFQKL | |
| | NPTLIGECEKAILWANEIVQKWLCTGMFAGQADAEAKAKRICDEL | |
| | NNHQATYAHARHIHLDKAQNIGLNIMELESDQTLQDLVLTIHHCY | |
| | MHSFGTSPAAKIIENHNGSTMMWNIC* | |
| ECOLIN_ | MSAAEFYEKMGIQPQEFQKGESVQHFAMRVLAQQNDLNVRSGVL | 255 |
| 10210 | YSYSTVTPNTTEQNGQQSHQLYSY* | |
| ECOLIN_ | MNQQDLNFVRIELRRALPDLSGGTKGQLEAFSEHPPADKNATPRR | 256 |
| 10220 | GIHLVELEGEKGPRFVNSLSAPLYVLETRSRRRPMPPIKDAEFESAP | |
| | WRRAVSALSGYQQAWLRYCYGFDLSYKHQVMMCEYVWKTYQK | |
| | CLGDNSLQERVVKKLIGLVWLAGQEIAATRNNETYKDYAGAALA | |
| | RMVSVDRSTWLRVYSGHWAGLKAAFTQLDESALAMALEYYEEE | |
| | EALKVAEM* | |
| ECOLIN_ | MRALLTPEIAHRMGIVLFRPGAELMHLFMRGRVLLEPEPEEMASFS | 257 |
| 10225 | TGAVPAAIQPLADDPVMRQVFENDRVIQRAGGLPSLEQWLSNRFE | |
| | CQWPHSSWHDKNFTTMRHPPGSIRLCWHCDHTLSGQHTEQLAGIA | |
| | AGNLVSWILEVIRRDSGFPESHILTLPELCWWMVRNDLADVIPESV | |
| | AHKGLRLPDDKIRSVMRESDIVPSASATSLVQEKAKKILTLSVDPES | |
| | PESFMLRPKRRRWINETYTRWVKTQPCECCRRPADDPHHIVGHGM | |
| | GGTATKAHDLFVIPLCRECHDELHADVPAFEQKHGTQLELLLRFM | |
| | DRALAIGVIAKA* | |
| ECOLIN_ | MSQLATTALTMSSSDIAELVESRHDHVKRSIERLAERGVIELPPMG | 258 |
| 10230 | EVKNHLNQSVSVYLIGKRDSYIVVAQLSPEFTARLVDRWQELEQA | |
| | QQQTIPQSFSEALRLAADLAEQKQQLTNELAAAAPKVAFVDRYCT | |
| | ASGSMSFRQVAKLLKAKEPDLRLFLLENDIMYRLGGTMTPRHQHI | |
| | DAGRFEVKTGTSVTSNHAFSQARFTAKGVRWIGGLWAEHIARGQ | |
| | VA* | |
| ECOLIN_ | MKLILPFPPSVNTYWRAPNKGPLAGRHLISAAGRKYQSAACVAIIE | 259 |
| 10235 | QLRRLPKPSTELAAVEITLYPPDARRRDIDNYNKALFDALTHAGV | |
| | WEDDSQIKRMLVEWGPVVPKGRVEITISRYEPAGAAA* | |
| ECOLIN_ | MMTNKYCQALAALRSKPAHELKEVGDQWRTPDLLFWGINALFGP | 260 |
| 10240 | LVLDLFADDDNAKCPAWYTAEDNALTQDWSERLAELGGAGYGN | |
| | PPYSRSQYHEKQAITGMTHIMNYAAAQREKGGRYVFLIKAAPSET | |
| | WWPEDADHIVFIRGRIGFDLPVWFVPADEKQKPTSAFFAGAIAVFD | |
| | KSWRGERFSYINRTELEAKGRAFMALAQFAASKSQPVTATPPAAD | |
| | KPEAELPLTQKDIFAISGVEAWACVRAAFGDKEEYTFSESKFGHTW | |
| | AADSVEAPEFTQVSPLTIDKAKLLIRESILFGVDEWLLSIEFDDAAV | |
| | RLDMSERIRTVALEASGEYGMNSTDFIAAMGSLDVSSWSNIRQIRM | |
| | HIREKAKPVSDPLPESRIWPLEVRIVFDQVDGADMLDESLQHKLKA | |
| | NINQLWLERTATSEIITAASELVRNMRGEAA* | |
| ECOLIN_ | MSTKLTGYVWDACASSGMKLSSVAIMARLADFSSDEGVSWPSIGT | 261 |
| 10245 | IARQIGAGESTVRTALAQLEKDGWLSRKQRRNGNRNASNVYQLN | |
| | VVKLREAAFSHLSESDASKSDPSKSDASKSDPSKFEASKSSKKGGF | |
| | DPSESGGDPSVKSKQEPQVTSKPSCPVAAQPDPEVVITDQARQVLS | |
| | YLNQTTGSRYQVCSTSLENIRARLREQFTVDDLCLVVDYKNADWR | |
| | DSEQAQYLRPATLFIPKNFPGYLQSATKWSSAGRPERVNGKWETN | |
| | SASRANFQSVDYSLPENSGFRS* | |
| ECOLIN_ | MNSVNRFRPAKQFRCLPLVGKDAQFGYVEIINNAADGGNYQPADL | 262 |
| 10250 | MVEAFVQMNEKGREEWLKLTGGSEITTEFPSELSAGSQIHSALYTF | |
| | AKGTIMSASALLNNSSVNLQN* | |
| ECOLIN_ | MVDSINTAIRLMCKAHKHGRLGMASDLGMTIDQFHNHLYQKCGS | 263 |
| 10255 | RFFTLAELERMEDLSGSCYLAEYQANRKGKWLVDVPTAESLDNV | |
| | ELYSIEMKAAAASGELANAKMAAAADGVIDSSERKMLSELFSKKL | |
| | RHQIHGFLGFMALYGVGVSDQAIDVFVSTGRKGDARECAAPGAL | |
| | ACRISGETNA* | |
| ECOLIN_ | MSSQHKNVTAKAVKAIGSISEVSRRFEFQSVQSVANWIAKNRVPSE | 264 |
| 10260 | RVIQLCQWGGWVVTPHQLRPDIYPNKNDGIPSANNNSQL* | |
| ECOLIN_ | MPCALNLLLMVENAKYKDFAERLNRSLQEQSIGVKELSEFSGVSY | 265 |
| 10265 | EMARRYTLGTAKPRDEKMIRIAERLAVSPAYLDYGVPVNGGDAPA | |
| | KGTVRIEQLDVHASAGSGYINQPFPTIVSSIEIPEERIFELFGRRSLDG | |
| | IVMINVDGDSMMPTLCPKDLLFIDSKVEQFSGDGVYVFNFEDSTFV | |
| | KRLQKVKGRRLAVLSDNEHYPPFFIEEHEMNELYIFGKLIRCLPLK | |
| | MIEFG* | |
| ECOLIN_ | MGAFDNQEITLPACPKCGTKTKKKIAWLKSNKSFTCRCGATINVN | 266 |
| 10270 | SSQLTSEIRKVEDKLKKLFK* | |
| ECOLIN_ | MNNPFFKNMLVYRISRDFTINQEELEQQLELFRFTPCGSQDMAKTG | 267 |
| 10275 | WVSPLGQLSDRLHHTVNNQVLLVIRREEKILPSPVITEELRKRVSRL | |
| | ESDQGRRLKKTEKDSLRDEVLHSLLPRAFSKNSTVGLWINVTDGLI | |
| | MVDAASAKRAEDSLALLRKTLGSLPVVPLTMETPIELTMTDWVRS | |
| | GSAPAGFGLGDEAELKAILEDGGIGRFKKQTLVSDEIHVHLEAGKV | |
| | VTKLSIDWQQRIQFVLCDDGSIKRLKFSNEITEQNDDIDREDAAQRF | |
| | DADFVLMTGELISLINGLTTSLGGEAKR* | |
| ECOLIN_ | MSYIQTLSGKHFNYLDIQQDDIVIEDIATALSHICRFAGHLPEFYSV | 268 |
| 10280 | GQHSVLTSHLVPQEFALEALLHDAAEAYLQDIPSPLKRLLPDYQAI | |
| | EARVDAAIRQKFGLPTEQHPTVKYADLVMLASERRDFEIDEGSIWP | |
| | CLEGVVPTDLFIINPVRPGQSYGMFINRFNELMEQRQCAA* | |
| ECOLIN_ | MTVFEYLQAHPNTTSGEIAKGMNKKTPAVAGALSQLYGTGRIVKS | 269 |
| 10290 | GVRKGIPTYRINDMPFGCSNSLTMMFNQLLSRARQGAAQ* | |
| ECOLIN_ | MTALNKQALREEFQFMQDNYSDPADHDRQVIYIEAEALLDELEAK | 270 |
| 10295 | DSTIAAQQHEIRMLLNALEEKPCPKCNDTGMTDSGGTQPWGEPIEI | |
| | ECDCRQQDANTAELVAAGIGVKGE* | |
| ECOLIN_ | MDKLIKPTAKGKYDGSCDYLCSEDARFIVMRGDYTEAEIIQASVSQ | 271 |
| 10300 | DVIDSDGAADFASSARYYQCWYKVSPIGGQDGYSGWHHPRDSPC | |
| | RGAYFASVLQWD* | |
| ECOLIN_ | MTTNNHPAHGPVSLDRLHQIREHLLHDTQYSNGGNRAYILADVLK | 272 |
| 10305 | VIDGAIARELVRREHAAWSQATFGDVGPVGPLKHLSKEALEAAAE | |
| | PGDLSEWADMQFLLWDAQRRAGISDEQITQAMIKKLAINKVRQW | |
| | PEPKDGEPRLHIKEQSEQEKK* | |
| ECOLIN_ | MFSLIRRGQIYTDSSNWPVIIHSCSDHSVRIKRNDGELRTISIKRFNE | 273 |
| 10310 | DFERVEHDEYRKICAEIEQETNLKNLRAMRRGKITE* | |
| ECOLIN_ | MNNLMIDLESMGKKPNAPIVSIGAVFFDPQSGELGQEFYTAVNLES | 274 |
| 10315 | AMEQGAVPDGDTILWWLRQSSEARSAICVDDAMPISSALSELSHFI | |
| | NRHSDNPKYLKVWGNGATFDNVILRGAYERAGQVCPWQFWNDH | |
| | DVRTIVTLGRSVGFDPKRDMPFDGVAHNALADARHQAKYVSAIW | |
| | QKLIPTTSNS* | |
| ECOLIN_ | MSNIFQLAPNDWVCESVLIAVTGLKPGTILRARKECWMIGREYIHV | 275 |
| 10320 | SPDGNPKPSSECMYNRKAVDAWVASMKSKQPG* | |
| ECOLIN_ | MDKVTYPTGVENHGGTLRIWFNFKGKRVRESLGVPDTAKNRKIA | 276 |
| 10325 | GELRTSVCFAIRTGTFDYATQFPDSPNLKAFGVSKKDITVKELEEK | |
| | WLDLKRMEICANAFNRYESVARNMVPRIGGNRLVSAVTKEELLYL | |
| | RKYLLTGYQNPTKNKAPAKGRSVVTVNYYMTTMAGMFQFAADH | |
| | GYLEVNPFEGIKPLKKARAEPDPLSRDEFIRLIDACRHQQTKNLWS | |
| | LAVYTGMRHGELVSLAWEDIDLKAGTITVRRNYTKLGEFTLPKTE | |
| | ASTDRVVHLIQPAISILKNQAEMTRLGRQYHIEVQLREYGRSVNHE | |
| | CTFVFNPHVVRRSKQVGFIYRVDSVGDSWEAALKRAGIRHRKAYQ | |
| | SRHTYACWSLSAGANPSFIASQMGHASAQMVFNVYGAWMADSS | |
| | AEQIAMLNQKLADFAPLMPHSHENSTGGLLKSVS* | |
| ECOLIN_ | MEGNTTLYALPKPEVVLRWREQTTDDFRFCFKFPATISHQAALRH | 277 |
| 10330 | CDDLVTEFLTRMSPLAPRIGQYWLQLPATFGPRELPALWHFLDSLP | |
| | GEFNYGVEVRHPQFFAKGEEEQTLNRGLHQRGVNRVILDSRPVHA | |
| | ARPYSEAIRDAQRKKPKVPVHAVLTAKNPLIRFIGSDDMTQNRELF | |
| | QVWLQKLAQWHQTTTPYLFLHTPDIAQAPELVHTLWEDLRKTLPE | |
| | IGAVPAIPQQSSLF* | |
| ECOLIN_ | MVSALYAVLSALLLMKFSFDVVRLRMQYRVAYGDGGFSELQSAI | 278 |
| 10335 | RIHGNAVEYIPIAIVLMLFMEMNGAETWMVHICGIVLLAGRLMHY | |
| | YGFHHRLFRWRRSGMSATWCALLLMVLANLWYMPWELVFSLR* | |
| ECOLIN_ | MSHRDTLFSAPIARLGDWTFDERVAEVFPDMIQRSVPGYSNIISMIG | 279 |
| 10340 | MLAERFVQPGTQVYDLGCSLGAATLSVRRNIHHDNCKIIAIDNSPA | |
| | MIERCRRHIDAYKAPTPVDVIEGDIRDIAIENASMVVLNFTLQFLEP | |
| | SERQALLDKIYQGLNPGGALVLSEKFSFEDAKVGELLFNMHHDFK | |
| | RANGYSELEISQKRSMLENVMLTDSVETHKARLHKAGFEHSELWF | |
| | QCFNFGSLVALKAEDAA* | |
| ECOLIN_ | MIDFGNFYSLIAKNHLSHWLETLPAQIANWQREQQHGLFKQWSNA | 280 |
| 10345 | VEFLPEIKPYRLDLLHSVTAESEEPLSAGQIKRIETLMRNLMPWRK | |
| | GPFSLYGVNIDTEWRSDWKWDRVMPHLSDLTGRTILDVGCGSGY | |
| | HMWRMIGAGAHLAVGIDPTQLFLCQFEAVRKLLGNDQRAHLLPL | |
| | GIEQLPALKAFDTVFSMGVLYHRRSPLEHLWQLKDQLVNEGELVL | |
| | ETLVIDGDENTVLVPGDRYAQMRNVYFIPSALALKNWLKKCGFV | |
| | DIRIADVSVTTTEEQRRTEWMVTESLADFLDPHDPGKTVEGYPAPK | |
| | RAVLIARKP* | |

In any of these embodiments, the bacteria described herein comprise one or more modifications or mutations within the E. coli Nissle Phage 3 genome. In some embodiments, the modifications alter the properties or behavior of the Phage 3. In some embodiments, the modifications or mutations prevent Phage 3 from entering or completing the lytic process. In some embodiments, the modifications or mutations reduce the ability of Phage 3 to enter the lytic process. In some embodiments, the modifications or mutations prevent the E. coli Nissle Phage 3 from infecting other bacteria of the same or a different type.

In some embodiments, the modifications or mutations alter, e.g., increase or reduce, the fitness of the bacterial host. In some embodiments, the modifications or mutations essentially have no effect on bacterial fitness, and the bacterial fitness is essentially the same as the fitness of the isogenic strain without the modifications or mutations. In some embodiments, the modifications or mutations alter, e.g., increase or reduce, the desired effector function, e.g., of a genetically engineered bacterium. In some embodiments, the modifications or mutations improve the desired effector function, e.g., of a genetically engineered bacterium. In some embodiments, the modifications or mutations essentially have no effect on effector function, and the effector function is essentially the same as effector function of the isogenic strain without the modifications or mutations. In some embodiments, the effector circuits are engineered into the genome first and then the phage is modified. In some embodiments, a new chassis with a modified Phage 3 is generated prior the engineering of the effector function(s).

In some embodiments, the bacteria comprise at least about 1 to 2, at least about 2 to 3, at least about 3 to 4, at least about 4 to 5, at least about 5 to 6, at least about 6 to 7, at least about 7 to 8, at least about 8 to 9, at least about 9 to 10, at least about 10 to 11, at least about 11 to 12, at least about 12 to 13, at least about 13 to 14, at least about 14 to 15, at least about 15 to 16, at least about 16 to 17, at least about 17 to 18, at least about 18 to 19, at least about 19 to 20, at least about 20 to 21, at least about 21 to 22, at least about 22 to 23, at least about 23 to 24, at least about 24 to 25, at least about 25 to 26, at least about 26 to 27, at least about 27 to 28, at least about 28 to 29, at least about 29 to 30, at least about 30 to 31, at least about 31 to 32, at least about 32 to 33, at least about 33 to 34, at least about 34 to 35, at least about 35 to 36, at least about 36 to 37, at least about 37 to 38, at least about 38 to 39, at least about 39 to 40, at least about 40 to 41, at least about 41 to 42, at least about 42 to 43, at least about 43 to 44, at least about 44 to 45, at least about 45 to 46, at least about 46 to 47, at least about 47 to 48, at least about 48 to 49, at least about 49 to 50, at least about 50 to 51, at least about 51 to 52, at least about 52 to 53, at least about 53 to 54, at least about 54 to 55, at least about 55 to 56, at least about 56 to 57, at least about 57 to 58, at least about 58 to 59, at least about 59 to 60, at least about 60 to 61, at least about 61 to 62, at least about 62 to 63, at least about 63 to 64, at least about 64 to 65, at least about 65 to 66, at least about 66 to 67, at least about 67 to 68, at least about 68 to 69, at least about 69 to 70, at least about 70 to 71, at least about 71 to 72, at least about 72 to 73, at least about 73 to 74, at least about 74 to 75, at least about 75 to 76, at least about 76 to 77, at least about 77 to 78, at least about 78 to 79, at least about 79 to 80, at least about 80 to 81, at least about 81 to 82, at least about 82 to 83, at least about 83 to 84, at least about 84 to 85, at least about 85 to 86, at least about 86 to 87, at least about 87 to 88, at least about 88 to 89, at least about 89 to 90, at least about 90 to 91, at least about 91 to 92, at least about 92 to 93, at least about 93 to 94, at least about 94 to 95, at least about 95 to 96, at least about 96 to 97, at least about 97 to 98, at least about 98 to 99, at least about 99 to 100, or at least about 100 or more modifications or mutations.

In some embodiments, the modifications or mutations reduce entry or completion of Phage 3 lytic process by at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold. In some embodiments, the modifications or mutations reduce entry or completion of Phage 3 lytic process completely.

In some embodiments, the modifications or mutations reduce entry or completion of Phage 3 lytic process by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100%.

In some embodiments, the modifications or mutations prevent E. coli Nissle Phage 3 genome from infecting other bacteria of the same or a different by at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold. In some embodiments, the modifications or mutations prevent the E. coli Nissle Phage 3 from infecting other bacteria of the same or a different type completely. In some embodiments, the modifications or mutations prevent the E. coli Nissle Phage 3 from infecting other bacteria of the same or a different type by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100%.

In some embodiments, the modifications or mutations alters, increases or reduces, the fitness of the bacterial host by at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold as compared to the same isogenic strain without the phage modification. In some embodiments, the modifications or mutations alters, increases or reduces, the fitness of the bacterial host by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100% as compared to the same isogenic strain without the phage modification.

In some embodiments, the modifications or mutations alters, e.g., increases or reduces, the desired effector function, e.g., of a genetically engineered bacterium by at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold as compared to the same isogenic strain without the phage modification. In some embodiments, the modifications or mutations alter, e.g., increase or reduce, the desired effector function, e.g., of a genetically engineered bacterium by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100% as compared to the same isogenic strain without the phage modification.

In some embodiments, the mutations include one or more deletions within the E. coli Nissle Phage 3 genome sequence. In some embodiments, the mutations include one or more insertions into the E. coli Nissle Phage 3 genome sequence. In some embodiments, an antibiotic cassette can be inserted into one or more positions within the E. coli Nissle Phage 3 genome sequence. In some embodiments, the mutations include one or more substitutions within the E. coli Nissle Phage 3 genome sequence. In some embodiments, the mutations include one or more inversions within the E. coli Nissle Phage 3 genome sequence. In some embodiments, the inversion may be governed by a specific flippase. Exemplary circuitry comprising multiple levels of control are exemplified herein and are also described in co-owned pending International Patent Application PCT/US2016/039434, the contents of which is herein incorporated by reference in its entirety.

In some embodiments, the modifications within the E. coli Nissle Phage 3 genome are combinations of two or more of insertions, deletions, substitutions, or inversions within one or more E. coli Nissle Phage 3 genome genes.

In any of the embodiments described herein, the modifications may result in one or more frameshift mutations in one or more genes within the E. coli Nissle Phage 3 genome. In any of the embodiments described herein, the modifications may result in one or more missense mutation in one or more genes within the E. coli Nissle Phage 3 genome. In any of the embodiments described herein, the modifications may result in one or more nonsense mutations in one or more genes within the E. coli Nissle Phage 3 genome.

In some embodiments, the modifications within the E. coli Nissle Phage 3 genome are combinations of two or more frameshift, nonsense or missense mutations within one or more E. coli Nissle Phage 3 genome genes.

### Mutations

In some embodiments, the one or more mutations comprise at least about 1-500 bp of the E. coli Nissle Phage 3 genome. In some embodiments, the one or more mutations comprise at least about 500-1000 bp of the E. coli Nissle Phage 3 genome. In some embodiments, the one or more mutations comprise at least about 1000-2000 bp of the E. coli Nissle Phage 3 genome. In some embodiments, the one or more mutations comprise at least about 1000-2000 bp of the E. coli Nissle Phage 3 genome. In some embodiments, the one or more mutations comprise at least about 2000-3000 bp of the E. coli Nissle Phage 3 genome. In some embodiments, the one or more mutations comprise at least about 3000-4000 bp of the E. coli Nissle Phage 3 genome. In some embodiments, the one or more mutations comprise at least about 4000-5000 bp of the E. coli Nissle Phage 3 genome. In some embodiments, the one or more mutations comprise at least about 5,000-6,000 bp of the E. coli Nissle Phage 3 genome. In some embodiments, the one or more mutations comprise at least about 6,000-7,000 bp of the E. coli Nissle Phage 3 genome. In some embodiments, the one or more mutations comprise at least about 7,000-8,000 bp of the E. coli Nissle Phage 3 genome. In some embodiments, the one or more mutations comprise at least about 8,000-9,000 bp of the E. coli Nissle Phage 3 genome. In some embodiments, the one or more mutations comprise at least about 9,000-10,000 bp of the E. coli Nissle Phage 3 genome. In some embodiments, the one or more mutations comprise at least about 10,000-15,000 bp of the E. coli Nissle Phage 3 genome. In some embodiments, the one or more mutations comprise at least about 10,000-15,000 bp of the E. coli Nissle Phage 3 genome, at least about 15,000-20,000 bp of the E. coli Nissle Phage 3 genome, at least about 20,000-25,000 bp of the E. coli Nissle Phage 3 genome, at least about 25,000-30,000 bp of the E. coli Nissle Phage 3 genome, at least about 30,000-35,000 bp of the E. coli Nissle Phage 3 genome, at least about 35,000-40,000 bp of the E. coli Nissle Phage 3 genome, at least about 40,000-45,000 bp of the E. coli Nissle Phage 3 genome, at least about 45,000-50,000 bp of the E. coli Nissle Phage 3 genome, at least about 50,000-55,000 bp of the E. coli Nissle Phage 3 genome, or at least about 55,000-60,000 bp of the E. coli Nissle Phage 3 genome. In one specific embodiment, 9687 bp of the E. coli Nissle Phage 3 genome are mutated. In some embodiments, the mutated nucleotides are interspersed. In some embodiments, the mutated nucleotides are consecutive.

In some embodiments, at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the E. coli Nissle Phage 3 genome is mutated. In some embodiments, at least about 30-40% of the E. coli Nissle Phage 3 genome is mutated. In some embodiments, at least about 40-50% of the E. coli Nissle Phage 3 genome is mutated. In some embodiments, at least about 50-60% of the E. coli Nissle Phage 3 genome is mutated. In some embodiments, at least about 60-70% of the E. coli Nissle Phage 3 genome is mutated. In some embodiments, at least about 70-80% of the E. coli Nissle Phage 3 genome is mutated. In some embodiments, at least about 80-90% of the E. coli Nissle Phage 3 genome is mutated. In some embodiments, 90-100% of the E. coli Nissle Phage 3 genome is mutated.

In some embodiments, at least about 1, 2, 3, 4, 5, 6, 7, 8, 9, 10, 11, 12, 13, 14, 15, 16, 17, 18, 19, or 20 genes are mutated. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes are mutated. In some embodiments, 13 genes are completely or partially mutated. In one embodiment, 74 genes are completely or partially mutated.

In some embodiments, at least about 1% to 2%, at least about 2% to 3%, at least about 3% to 4%, at least about 4% to 5%, at least about 5% to 6%, at least about 6% to 7%, at least about 7% to 8%, at least about 8% to 9%, at least about 9% to 10%, at least about 10% to 11%, at least about 11% to 12%, at least about 12% to 13%, at least about 13% to 14%, at least about 14% to 15%, at least about 15% to 16%, at least about 16% to 17%, at least about 17% to 18%, at least about 18% to 19%, at least about 19% to 20%, at least about 20% to 21%, at least about 21% to 22%, at least about 22% to 23%, at least about 23% to 24%, at least about 24% to 25%, at least about 25% to 26%, at least about 26% to 27%, at least about 27% to 28%, at least about 28% to 29%, at least about 29% to 30%, at least about 30% to 31%, at least about 31% to 32%, at least about 32% to 33%, at least about 33% to 34%, at least about 34% to 35%, at least about 35% to 36%, at least about 36% to 37%, at least about 37% to 38%, at least about 38% to 39%, at least about 39% to 40%, at least about 40% to 41%, at least about 41% to 42%, at least about 42% to 43%, at least about 43% to 44%, at least about 44% to 45%, at least about 45% to 46%, at least about 46% to 47%, at least about 47% to 48%, at least about 48% to 49%, at least about 49% to 50%, at least about 50% to 51%, at least about 51% to 52%, at least about 52% to 53%, at least about 53% to 54%, at least about 54% to 55%, at least about 55% to 56%, at least about 56% to 57%, at least about 57% to 58%, at least about 58% to 59%, at least about 59% to 60%, at least about 60% to 61%, at least about 61% to 62%, at least about 62% to 63%, at least about 63% to 64%, at least about 64% to 65%, at least about 65% to 66%, at least about 66% to 67%, at least about 67% to 68%, at least about 68% to 69%, at least about 69% to 70%, at least about 70% to 71%, at least about 71% to 72%, at least about 72% to 73%, at least about 73% to 74%, at least about 74% to 75%, at least about 75% to 76%, at least about 76% to 77%, at least about 77% to 78%, at least about 78% to 79%, at least about 79% to 80%, at least about 80% to 81%, at least about 81% to 82%, at least about 82% to 83%, at least about 83% to 84%, at least about 84% to 85%, at least about 85% to 86%, at least about 86% to 87%, at least about 87% to 88%, at least about 88% to 89%, at least about 89% to 90%, at least about 90% to 91%, at least about 91% to 92%, at least about 92% to 93%, at least about 93% to 94%, at least about 94% to 95%, at least about 95% to 96%, at least about 96% to 97%, at least about 97% to 98%, at least about 98% to 99%, at least about 99% to 100%, or at least about 100% of genes within the E. coli Nissle Phage 3 genome are completely or partially mutated.

In some embodiments, the one or more mutations are located at the beginning or 5′ end of the E. coli Nissle Phage 3 genome. In some embodiments, the one or more mutations are located at the end or 3′ end of the E. coli Nissle Phage 3 genome. In some embodiments, the one or more mutations are located in the middle of the E. coli Nissle Phage 3 genome. In some embodiments, the E. coli Nissle Phage 3 genes are interspersed within the bacterial genome and the mutation are located in one or more of the interspersed positions.

In some embodiments, the mutations are located within or encompass one or more genes encoding lytic genes. In some embodiments, the mutations are located within or encompass one or more genes encoding one or more proteases or lysins. In some embodiments, the mutations are located within or encompass one or more genes encoding one or more toxins. In some embodiments, the mutations are located within or encompass one or more genes encoding one or more antibiotic resistance related proteins. In some embodiments, the mutations are located within or encompass one or more genes encoding one or phage translation related proteins. In some embodiments, the one or more mutations are located within or encompass one or more genes encoding structural proteins. Such structural genes include genes encoding polypeptides of the head, tail, collar, or coat. In some embodiments, the one or more mutations are located within or encompass one or more genes encoding head proteins. In some embodiments, the one or more mutations are located within or encompass one or more genes encoding tail proteins. In some embodiments, the one or more mutations are located within or encompass one or more genes encoding collar proteins. In some embodiments, the one or more mutations are located within or encompass one or more genes encoding coat proteins. In some embodiments, the mutations are located within or encompass one or more genes encoding one or more plate proteins. In some embodiments, the mutations are located within or encompass one or more genes encoding one or more proteins require for assembly of the bacteriophage. In some embodiments, the mutations are located within or encompass one or more genes encoding one or more portal proteins. In some embodiments, the mutations are located within or encompass one or more genes encoding one or more polypeptides involved in recombination. In some embodiments, the mutations are located within or encompass one or more genes encoding one or more integrases. In some embodiments, the mutations are located within or encompass one or more genes encoding one or more invertases. In some embodiments, the mutations are located within or encompass one or more genes encoding one or more transposases. In some embodiments, the mutations are located with within or encompass one or more genes encoding one or more polypeptides involved in replication or translation. In some embodiments, the mutations are located within or encompass one or more genes encoding one or more primases. In some embodiments, the mutations are located within or encompass one or more genes encoding one or more tRNA related proteins. In some embodiments, the mutations are located within or encompass one or more genes encoding one or more polypeptides involved in phage insertion. In some embodiments, the mutations are located within or encompass one or more genes encoding an attachment site. In some embodiments, the mutations are located within or encompass one or more genes encoding one or more polypeptides involved in packaging. In some embodiments, the mutations are located within or encompass one or more genes encoding one or more terminases. In some embodiments, the mutations are located within or encompass one or more genes encoding one or more host genes.

In some embodiments, the mutations are located withinor encompass genes encoding one or more polypeptides involved in one or more of cell lysis, phage structure, phage assembly, phage packaging recombination, replication or translation, phage insertion, or are host proteins, and combinations thereof.

In some embodiments, the mutations are located withinor encompass genes encoding one or more polypeptides involved in one or more of cell lysis, phage structure, phage assembly, phage packaging recombination, replication or translation, phage insertion, and combinations thereof.

In some embodiments, the mutations are located withinor encompass 1 genes encoding polypeptides involved in cell lysis, phage structure, phage assembly, phage packaging recombination, replication or translation, phage insertion, and combinations thereof. In some embodiments, the mutations are located withinor encompass 2 genes encoding polypeptides involved in cell lysis, phage structure, phage assembly, phage packaging recombination, replication or translation, phage insertion, and combinations thereof. In some embodiments, the mutations are located withinor encompass 3 genes encoding polypeptides involved in cell lysis, phage structure, phage assembly, phage packaging recombination, replication or translation, phage insertion, and combinations thereof. In some embodiments, the mutations are located withinor encompass 4 genes encoding polypeptides involved in cell lysis, phage structure, phage assembly, phage packaging recombination, replication or translation, phage insertion, and combinations thereof. In some embodiments, the mutations are located withinor encompass 2 genes encoding polypeptides involved in cell lysis, phage structure, phage assembly, phage packaging recombination, replication or translation, phage insertion, and combinations thereof. In some embodiments, the mutations are located withinor encompass 5 genes encoding polypeptides involved in cell lysis, phage structure, phage assembly, phage packaging recombination, replication or translation, phage insertion, and combinations thereof. In some embodiments, the mutations are located withinor encompass 6 genes encoding polypeptides involved in cell lysis, phage structure, phage assembly, phage packaging recombination, replication or translation, phage insertion, and combinations thereof. In some embodiments, the mutations are located withinor encompass 7 genes encoding polypeptides involved in cell lysis, phage structure, phage assembly, phage packaging recombination, replication or translation, phage insertion, and combinations thereof. In some embodiments, the mutations are located withinor encompass 8 genes encoding polypeptides involved in cell lysis, phage structure, phage assembly, phage packaging recombination, replication or translation, phage insertion, and combinations thereof. In some embodiments, the mutations are located withinor encompass 9 genes encoding polypeptides involved in cell lysis, phage structure, phage assembly, phage packaging recombination, replication or translation, phage insertion, and combinations thereof. In some embodiments, the mutations are located withinor encompass 10 genes encoding polypeptides involved in cell lysis, phage structure, phage assembly, phage packaging recombination, replication or translation, phage insertion, and combinations thereof. In some embodiments, the mutations are located withinor encompass 11 genes encoding polypeptides involved in cell lysis, phage structure, phage assembly, phage packaging recombination, replication or translation, phage insertion, and combinations thereof. In some embodiments, the mutations are located withinor encompass 12 genes encoding polypeptides involved in cell lysis, phage structure, phage assembly, phage packaging recombination, replication or translation, phage insertion, and combinations thereof. In some embodiments, the mutations are located withinor encompass 13 genes encoding polypeptides involved in cell lysis, phage structure, phage assembly, phage packaging recombination, replication or translation, phage insertion, and combinations thereof. In some embodiments, the mutations are located withinor encompass 14 genes encoding polypeptides involved in cell lysis, phage structure, phage assembly, phage packaging recombination, replication or translation, phage insertion, and combinations thereof. In some embodiments, the mutations are located withinor encompass 15 genes encoding polypeptides involved in cell lysis, phage structure, phage assembly, phage packaging recombination, replication or translation, phage insertion, and combinations thereof. In some embodiments, the mutations are located withinor encompass at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the mutations are located withinor encompass one or more host proteins within the phage genome.

In any of the embodiments described herein, the modifications encompass are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In some embodiments, one or more mutations encompass or are located in ECOLIN_09965. In some embodiments, one or more mutations encompass or are located in ECOLIN_09970. In some embodiments, one or more mutations encompass or are located in ECOLIN_09975. In some embodiments, one or more mutations encompass or are located in ECOLIN_09980. In some embodiments, one or more mutations encompass or are located in ECOLIN_09985. In some embodiments, one or more mutations encompass or are located in ECOLIN_09990. In some embodiments, one or more mutations encompass or are located in ECOLIN_09995. In some embodiments, one or more mutations encompass or are located in ECOLIN_10000. In some embodiments, one or more mutations encompass or are located in ECOLIN_10005. In some embodiments, one or more mutations encompass or are located in ECOLIN_10010. In some embodiments, one or more mutations encompass or are located in ECOLIN_10015. In some embodiments, one or more mutations encompass or are located in ECOLIN_10020. In some embodiments, one or more mutations encompass or are located in ECOLIN_10025. In some embodiments, one or more mutations encompass or are located in ECOLIN_10030. In some embodiments, one or more mutations encompass or are located in ECOLIN_10035. In some embodiments, one or more mutations encompass or are located in ECOLIN_10040. In some embodiments, one or more mutations encompass or are located in ECOLIN_10045. In some embodiments, one or more mutations encompass or are located in ECOLIN_10050. In some embodiments, one or more mutations encompass or are located in ECOLIN_10055. In some embodiments, one or more mutations encompass or are located in ECOLIN_10065. In some embodiments, one or more mutations encompass or are located in ECOLIN_10070. In some embodiments, one or more mutations encompass or are located in ECOLIN_10075. In some embodiments, one or more mutations encompass or are located in ECOLIN_10080. In some embodiments, one or more mutations encompass or are located in ECOLIN_10085. In some embodiments, one or more mutations encompass or are located in ECOLIN_10090. In some embodiments, one or more mutations encompass or are located in ECOLIN_10095. In some embodiments, one or more mutations encompass or are located in ECOLIN_10100. In some embodiments, one or more mutations encompass or are located in ECOLIN_10105. In some embodiments, one or more mutations encompass or are located in ECOLIN_10110. In some embodiments, one or more mutations encompass or are located in ECOLIN_10115. In some embodiments, one or more mutations encompass or are located in ECOLIN_10120. In some embodiments, one or more mutations encompass or are located in ECOLIN_10125. In some embodiments, one or more mutations encompass or are located in ECOLIN_10130. In some embodiments, one or more mutations encompass or are located in ECOLIN_10135. In some embodiments, one or more mutations encompass or are located in ECOLIN_10140. In some embodiments, one or more mutations encompass or are located in ECOLIN_10145. In some embodiments, one or more mutations encompass or are located in ECOLIN_10150. In some embodiments, one or more mutations encompass or are located in ECOLIN_10160. In some embodiments, one or more mutations encompass or are located in ECOLIN_10165. In some embodiments, one or more mutations encompass or are located in ECOLIN_10170. In some embodiments, one or more mutations encompass or are located in ECOLIN_10175. In some embodiments, one or more mutations encompass or are located in ECOLIN_10180. In some embodiments, one or more mutations encompass or are located in ECOLIN_10185. In some embodiments, one or more mutations encompass or are located in ECOLIN_10190. In some embodiments, one or more mutations encompass or are located in ECOLIN_10195. In some embodiments, one or more mutations encompass or are located in ECOLIN_10200. In some embodiments, one or more mutations encompass or are located in ECOLIN_10205. In some embodiments, one or more mutations encompass or are located in ECOLIN_10210. In some embodiments, one or more mutations encompass or are located in ECOLIN_10220. In some embodiments, one or more mutations encompass or are located in ECOLIN_10225. In some embodiments, one or more mutations encompass or are located in ECOLIN_10230. In some embodiments, one or more mutations encompass or are located in ECOLIN_10235. In some embodiments, one or more mutations encompass or are located in ECOLIN_10240. In some embodiments, one or more mutations encompass or are located in ECOLIN_10245. In some embodiments, one or more mutations encompass or are located in ECOLIN_10250. In some embodiments, one or more mutations encompass or are located in ECOLIN_10255. In some embodiments, one or more mutations encompass or are located in ECOLIN_10260. In some embodiments, one or more mutations encompass or are located in ECOLIN_10265. In some embodiments, one or more mutations encompass or are located in ECOLIN_10270. In some embodiments, one or more mutations encompass or are located in ECOLIN_10275. In some embodiments, one or more mutations encompass or are located in ECOLIN_10280. In some embodiments, one or more mutations encompass or are located in ECOLIN_10290. In some embodiments, one or more mutations encompass or are located in ECOLIN_10295. In some embodiments, one or more mutations encompass or are located in ECOLIN_10300. In some embodiments, one or more mutations encompass or are located in ECOLIN_10305. In some embodiments, one or more mutations encompass or are located in ECOLIN_10310. In some embodiments, one or more mutations encompass or are located in ECOLIN_10315. In some embodiments, one or more mutations encompass or are located in ECOLIN_10320. In some embodiments, one or more mutations encompass or are located in ECOLIN_10325. In some embodiments, one or more mutations encompass or are located in ECOLIN_10330. In some embodiments, one or more mutations encompass or are located in ECOLIN_10335. In some embodiments, one or more mutations encompass or are located in ECOLIN_10340. In some embodiments, one or more mutations encompass or are located in ECOLIN_10345.

In some embodiments, the mutations are located in or encompass one or more polypeptides selected from lipid A biosynthesis (KDO)2-(lauroyl)-lipid IVA acyltransferase, peptidase, zinc ABC transporter substrate-binding protein, zinc ABC transporter ATPase, high-affinity zinc transporter membrane component, ATP-dependent DNA helicase RuvB, ATP-dependent DNA helicase RuvA, Holliday junction resolvase, dihydroneopterin triphosphate pyrophosphatase, aspartyl-tRNA synthetase, hydrolase, DNA polymerase V, MsgA, phage tail protein, tail protein, host specificity protein, peptidase P60, tail protein, tail protein, tail fiber protein, Minor tail protein U, DNA breaking-rejoining protein, peptidase S14, capsid protein, DNA packaging protein, terminase, lysozyme, holin, DNA adenine methylase, serine protease, antitermination protein, antirepressor, crossover junction endodeoxyribonuclease, adenine methyltransferase, DNA methyltransferase ECOLIN_10240, GntR family transcriptional regulator ECOLIN_10245, cI repressor, Domain of unknown function (DUF4222); DNA recombinase, Multiple Antibiotic Resistance Regulator (MarR), unknown ead like protein in P22, Protein of unknown function (DUF550); 3′-5′ exonuclease, excisionase, integrase, and tRNA methyltransferase. In one embodiment, one or more of a Minor tail protein U, a tail protein, a DNA breaking-rejoining protein, a peptidase S14, a capsid protein, a DNA packaging protein, and a terminase are mutated.

In one embodiment, the mutation is a complete or partial mutation of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the mutation is a complete or partial mutation of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the mutation is a complete mutation of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial mutation of ECOLIN_10175. In one embodiment, the sequence of SEQ ID NO: 130 is mutated from the Phage 3 genome. In one embodiment, a sequence comprising SEQ ID NO: 130 is mutated from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Deletions

In some embodiments, the one or more deletions comprise at least about 1-500 bp of the E. coli Nissle Phage 3 genome. In some embodiments, the one or more deletions comprise at least about 500-1000 bp of the E. coli Nissle Phage 3 genome. In some embodiments, the one or more deletions comprise at least about 1000-2000 bp of the E. coli Nissle Phage 3 genome. In some embodiments, the one or more deletions comprise at least about 1000-2000 bp of the E. coli Nissle Phage 3 genome. In some embodiments, the one or more deletions comprise at least about 2000-3000 bp of the E. coli Nissle Phage 3 genome. In some embodiments, the one or more deletions comprise at least about 3000-4000 bp of the E. coli Nissle Phage 3 genome. In some embodiments, the one or more deletions comprise at least about 4000-5000 bp of the E. coli Nissle Phage 3 genome. In some embodiments, the one or more deletions comprise at least about 5,000-6,000 bp of the E. coli Nissle Phage 3 genome. In some embodiments, the one or more deletions comprise at least about 6,000-7,000 bp of the E. coli Nissle Phage 3 genome. In some embodiments, the one or more deletions comprise at least about 7,000-8,000 bp of the E. coli Nissle Phage 3 genome. In some embodiments, the one or more deletions comprise at least about 8,000-9,000 bp of the E. coli Nissle Phage 3 genome. In some embodiments, the one or more deletions comprise at least about 9,000-10,000 bp of the E. coli Nissle Phage 3 genome. In some embodiments, the one or more deletions comprise at least about 10,000-15,000 bp of the E. coli Nissle Phage 3 genome. In some embodiments, the one or more deletions comprise at least about 10,000-15,000 bp of the E. coli Nissle Phage 3 genome, at least about 15,000-20,000 bp of the E. coli Nissle Phage 3 genome, at least about 20,000-25,000 bp of the E. coli Nissle Phage 3 genome, at least about 25,000-30,000 bp of the E. coli Nissle Phage 3 genome, at least about 30,000-35,000 bp of the E. coli Nissle Phage 3 genome, at least about 35,000-40,000 bp of the E. coli Nissle Phage 3 genome, at least about 40,000-45,000 bp of the E. coli Nissle Phage 3 genome, at least about 45,000-50,000 bp of the E. coli Nissle Phage 3 genome, at least about 50,000-55,000 bp of the E. coli Nissle Phage 3 genome, or at least about 55,000-60,000 bp of the E. coli Nissle Phage 3 genome. In one specific embodiment, 9687 bp of the E. coli Nissle Phage 3 genome are deleted. In some embodiments, the deleted nucleotides are interspersed. In some embodiments, the deleted nucleotides are consecutive.

In some embodiments, at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the E. coli Nissle Phage 3 genome is deleted. In some embodiments, at least about 30-40% of the E. coli Nissle Phage 3 genome is deleted. In some embodiments, at least about 40-50% of the E. coli Nissle Phage 3 genome is deleted. In some embodiments, at least about 50-60% of the E. coli Nissle Phage 3 genome is deleted. In some embodiments, at least about 60-70% of the E. coli Nissle Phage 3 genome is deleted. In some embodiments, at least about 70-80% of the E. coli Nissle Phage 3 genome is deleted. In some embodiments, at least about 80-90% of the E. coli Nissle Phage 3 genome is deleted. In some embodiments, at least about 90-100% of the E. coli Nissle Phage 3 genome is deleted.

In some embodiments, one or more genes are partially or completely deleted within the E. coli Nissle Phage 3 genome. In some embodiments, one or more genes are completely deleted and one or more genes are partially deleted. In one embodiment, there is one deletion within the E. coli Nissle Phage 3 genome and one or two genes at the ends of the deletion are partially deleted and the rest of the genes are completely deleted. In some embodiments, the deleted genes are adjacent to each other. In some embodiments, the deleted genes are not adjacent to each other.

In some embodiments, at least about 1, 2, 3, 4, 5, 6, 7, 8, 9, 10, 11, 12, 13, 14, 15, 16, 17, 18, 19, or 20 genes are deleted. In some embodiments, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes are deleted. In some embodiments, 13 genes are completely or partially deleted. In one embodiment, 74 genes are completely or partially deleted.

In some embodiments, at least about 1% to 2%, at least about 2% to 3%, at least about 3% to 4%, at least about 4% to 5%, at least about 5% to 6%, at least about 6% to 7%, at least about 7% to 8%, at least about 8% to 9%, at least about 9% to 10%, at least about 10% to 11%, at least about 11% to 12%, at least about 12% to 13%, at least about 13% to 14%, at least about 14% to 15%, at least about 15% to 16%, at least about 16% to 17%, at least about 17% to 18%, at least about 18% to 19%, at least about 19% to 20%, at least about 20% to 21%, at least about 21% to 22%, at least about 22% to 23%, at least about 23% to 24%, at least about 24% to 25%, at least about 25% to 26%, at least about 26% to 27%, at least about 27% to 28%, at least about 28% to 29%, at least about 29% to 30%, at least about 30% to 31%, at least about 31% to 32%, at least about 32% to 33%, at least about 33% to 34%, at least about 34% to 35%, at least about 35% to 36%, at least about 36% to 37%, at least about 37% to 38%, at least about 38% to 39%, at least about 39% to 40%, at least about 40% to 41%, at least about 41% to 42%, at least about 42% to 43%, at least about 43% to 44%, at least about 44% to 45%, at least about 45% to 46%, at least about 46% to 47%, at least about 47% to 48%, at least about 48% to 49%, at least about 49% to 50%, at least about 50% to 51%, at least about 51% to 52%, at least about 52% to 53%, at least about 53% to 54%, at least about 54% to 55%, at least about 55% to 56%, at least about 56% to 57%, at least about 57% to 58%, at least about 58% to 59%, at least about 59% to 60%, at least about 60% to 61%, at least about 61% to 62%, at least about 62% to 63%, at least about 63% to 64%, at least about 64% to 65%, at least about 65% to 66%, at least about 66% to 67%, at least about 67% to 68%, at least about 68% to 69%, at least about 69% to 70%, at least about 70% to 71%, at least about 71% to 72%, at least about 72% to 73%, at least about 73% to 74%, at least about 74% to 75%, at least about 75% to 76%, at least about 76% to 77%, at least about 77% to 78%, at least about 78% to 79%, at least about 79% to 80%, at least about 80% to 81%, at least about 81% to 82%, at least about 82% to 83%, at least about 83% to 84%, at least about 84% to 85%, at least about 85% to 86%, at least about 86% to 87%, at least about 87% to 88%, at least about 88% to 89%, at least about 89% to 90%, at least about 90% to 91%, at least about 91% to 92%, at least about 92% to 93%, at least about 93% to 94%, at least about 94% to 95%, at least about 95% to 96%, at least about 96% to 97%, at least about 97% to 98%, at least about 98% to 99%, at least about 99% to 100%, or at least about 100% of genes within the E. coli Nissle Phage 3 genome are completely or partially deleted.

In some embodiments, the one or more deletions are located at the beginning or 5′ end of the E. coli Nissle Phage 3 genome. In some embodiments, the one or more deletions are located at the end or 3′ end of the E. coli Nissle Phage 3 genome. In some embodiments, the one or more deletions are located in the middle of the E. coli Nissle Phage 3 genome. In some embodiments, the E. coli Nissle Phage 3 genes are interspersed within the bacterial genome and the deletion are located in one or more of the interspersed positions.

In some embodiments, the region for an optimal deletion, i.e., to achieve a desired effect, can be determined through analysis of homology with other phages in other bacteria, e.g., other E. coli strains. Homologous conserved regions in E. coli Nissle Phage 3 may be suitable for deletion, as these are conserved and may comprise one or more essential genes. In some embodiments, regulatory elements, such as promoters, are deleted. In some embodiments, coding sequences are deleted. In some embodiments, the one or more deleted regions contain one or more genes essential for the lytic cycle.

In some embodiments, the deletions are located within or encompass one or more genes encoding lytic genes. In some embodiments, the deletions are located within or encompass one or more genes encoding one or more proteases or lysins. In some embodiments, the deletions are located within or encompass one or more genes encoding one or more toxins. In some embodiments, the deletions are located within or encompass one or more genes encoding one or more antibiotic resistance related proteins. In some embodiments, the deletions are located within or encompass one or more genes encoding one or phage translation related proteins. In some embodiments, the one or more deletions are located within or encompass one or more genes encoding structural proteins. Such structural genes include genes encoding polypeptides of the head, tail, collar, or coat. In some embodiments, the deletions are located within or encompass one or more genes encoding one or more head proteins. In some embodiments, the deletions are located within or encompass one or more genes encoding one or more tail proteins. In some embodiments, the deletions are located within or encompass one or more genes encoding one or more collar proteins. In some embodiments, the deletions are located within or encompass one or more genes encoding one or more coat proteins. In some embodiments, the deletions are located within or encompass one or more genes encoding one or more plate proteins. In some embodiments, the deletions are located within or encompass one or more genes encoding one or more proteins require for assembly of the bacteriophage. In some embodiments, the deletions are located within or encompass one or more genes encoding one or more portal proteins. In some embodiments, the deletions are located within or encompass one or more genes encoding one or more polypeptides involved in recombination. In some embodiments, the deletions are located within or encompass one or more genes encoding one or more integrases. In some embodiments, the deletions are located within or encompass one or more genes encoding one or more invertases. In some embodiments, the deletions are located within or encompass one or more genes encoding one or more transposases. In some embodiments, the deletions are located with within or encompass one or more genes encoding one or more polypeptides involved in replication or translation. In some embodiments, the deletions are located within or encompass one or more genes encoding one or more primases. In some embodiments, the deletions are located within or encompass one or more genes encoding one or more tRNA related proteins. In some embodiments, the deletions are located within or encompass one or more genes encoding one or more polypeptides involved in phage insertion. In some embodiments, the deletions are located within or encompass one or more genes encoding an attachment site. In some embodiments, the deletions are located within or encompass one or more genes encoding one or more polypeptides involved in packaging. In some embodiments, the deletions are located within or encompass one or more genes encoding one or more terminases. In some embodiments, the deletions are located within or encompass one or more genes encoding one or more host genes.

In some embodiments, the deletions are located withinor encompass genes encoding one or more polypeptides involved in one or more of cell lysis, phage structure, phage assembly, phage packaging recombination, replication or translation, phage insertion, or are host proteins, and combinations thereof.

In some embodiments, the deletions are located withinor encompass genes encoding one or more polypeptides involved in one or more of cell lysis, phage structure, phage assembly, phage packaging recombination, replication or translation, phage insertion, and combinations thereof.

In some embodiments, the deletions are located withinor encompass 1 genes encoding polypeptides involved in cell lysis, phage structure, phage assembly, phage packaging recombination, replication or translation, phage insertion, and combinations thereof. In some embodiments, the deletions are located withinor encompass 2 genes encoding polypeptides involved in cell lysis, phage structure, phage assembly, phage packaging recombination, replication or translation, phage insertion, and combinations thereof. In some embodiments, the deletions are located withinor encompass 3 genes encoding polypeptides involved in cell lysis, phage structure, phage assembly, phage packaging recombination, replication or translation, phage insertion, and combinations thereof. In some embodiments, the deletions are located withinor encompass 4 genes encoding polypeptides involved in cell lysis, phage structure, phage assembly, phage packaging recombination, replication or translation, phage insertion, and combinations thereof. In some embodiments, the deletions are located withinor encompass 2 genes encoding polypeptides involved in cell lysis, phage structure, phage assembly, phage packaging recombination, replication or translation, phage insertion, and combinations thereof. In some embodiments, the deletions are located within or encompass 5 genes encoding polypeptides involved in cell lysis, phage structure, phage assembly, phage packaging recombination, replication or translation, phage insertion, and combinations thereof. In some embodiments, the deletions are located within or encompass 6 genes encoding polypeptides involved in cell lysis, phage structure, phage assembly, phage packaging recombination, replication or translation, phage insertion, and combinations thereof. In some embodiments, the deletions are located within or encompass 7 genes encoding polypeptides involved in cell lysis, phage structure, phage assembly, phage packaging recombination, replication or translation, phage insertion, and combinations thereof. In some embodiments, the deletions are located within or encompass 8 genes encoding polypeptides involved in cell lysis, phage structure, phage assembly, phage packaging recombination, replication or translation, phage insertion, and combinations thereof. In some embodiments, the deletions are located within or encompass 9 genes encoding polypeptides involved in cell lysis, phage structure, phage assembly, phage packaging recombination, replication or translation, phage insertion, and combinations thereof. In some embodiments, the deletions are located within or encompass 10 genes encoding polypeptides involved in cell lysis, phage structure, phage assembly, phage packaging recombination, replication or translation, phage insertion, and combinations thereof. In some embodiments, the deletions are located within or encompass 11 genes encoding polypeptides involved in cell lysis, phage structure, phage assembly, phage packaging recombination, replication or translation, phage insertion, and combinations thereof. In some embodiments, the deletions are located within or encompass 12 genes encoding polypeptides involved in cell lysis, phage structure, phage assembly, phage packaging recombination, replication or translation, phage insertion, and combinations thereof. In some embodiments, the deletions are located within or encompass 13 genes encoding polypeptides involved in cell lysis, phage structure, phage assembly, phage packaging recombination, replication or translation, phage insertion, and combinations thereof. In some embodiments, the deletions are located within or encompass 14 genes encoding polypeptides involved in cell lysis, phage structure, phage assembly, phage packaging recombination, replication or translation, phage insertion, and combinations thereof. In some embodiments, the deletions are located within or encompass 15 genes encoding polypeptides involved in cell lysis, phage structure, phage assembly, phage packaging recombination, replication or translation, phage insertion, and combinations thereof. In some embodiments, the deletions are located within or encompass at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the deletions are located within or encompass one or more host proteins within the phage genome.

In any of the embodiments described herein, the deletions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In some embodiments, one or more deletions encompass (completely or partially) or are located in ECOLIN_09965. In some embodiments, one or more deletions encompass (completely or partially) or are located in ECOLIN_09970. In some embodiments, one or more deletions encompass (completely or partially) or are located in ECOLIN_09975. In some embodiments, one or more deletions encompass (completely or partially) or are located in ECOLIN_09980. In some embodiments, one or more deletions encompass (completely or partially) or are located in ECOLIN_09985. In some embodiments, one or more deletions encompass (completely or partially) or are located in ECOLIN_09990. In some embodiments, one or more deletions encompass (completely or partially) or are located in ECOLIN_09995. In some embodiments, one or more deletions encompass (completely or partially) or are located in ECOLIN_10000. In some embodiments, one or more deletions encompass (completely or partially) or are located in ECOLIN_10005. In some embodiments, one or more deletions encompass (completely or partially) or are located in ECOLIN_10010. In some embodiments, one or more deletions encompass (completely or partially) or are located in ECOLIN_10015. In some embodiments, one or more deletions encompass (completely or partially) or are located in ECOLIN_10020. In some embodiments, one or more deletions encompass (completely or partially) or are located in ECOLIN_10025. In some embodiments, one or more deletions encompass (completely or partially) or are located in ECOLIN_10030. In some embodiments, one or more deletions encompass (completely or partially) or are located in ECOLIN_10035. In some embodiments, one or more deletions encompass (completely or partially) or are located in ECOLIN_10040. In some embodiments, one or more deletions encompass (completely or partially) or are located in ECOLIN_10045. In some embodiments, one or more deletions encompass (completely or partially) or are located in ECOLIN_10050. In some embodiments, one or more deletions encompass (completely or partially) or are located in ECOLIN_10055. In some embodiments, one or more deletions encompass (completely or partially) or are located in ECOLIN_10065. In some embodiments, one or more deletions encompass (completely or partially) or are located in ECOLIN_10070. In some embodiments, one or more deletions encompass (completely or partially) or are located in ECOLIN_10075. In some embodiments, one or more deletions encompass (completely or partially) or are located in ECOLIN_10080. In some embodiments, one or more deletions encompass (completely or partially) or are located in ECOLIN_10085. In some embodiments, one or more deletions encompass (completely or partially) or are located in ECOLIN_10090. In some embodiments, one or more deletions encompass (completely or partially) or are located in ECOLIN_10095. In some embodiments, one or more deletions encompass (completely or partially) or are located in ECOLIN_10100. In some embodiments, one or more deletions encompass (completely or partially) or are located in ECOLIN_10105. In some embodiments, one or more deletions encompass (completely or partially) or are located in ECOLIN_10110. In some embodiments, one or more deletions encompass (completely or partially) or are located in ECOLIN_10115. In some embodiments, one or more deletions encompass (completely or partially) or are located in ECOLIN_10120. In some embodiments, one or more deletions encompass (completely or partially) or are located in ECOLIN_10125. In some embodiments, one or more deletions encompass (completely or partially) or are located in ECOLIN_10130. In some embodiments, one or more deletions encompass (completely or partially) or are located in ECOLIN_10135. In some embodiments, one or more deletions encompass (completely or partially) or are located in ECOLIN_10140. In some embodiments, one or more deletions encompass (completely or partially) or are located in ECOLIN_10145. In some embodiments, one or more deletions encompass (completely or partially) or are located in ECOLIN_10150. In some embodiments, one or more deletions encompass (completely or partially) or are located in ECOLIN_10160. In some embodiments, one or more deletions encompass (completely or partially) or are located in ECOLIN_10165. In some embodiments, one or more deletions encompass (completely or partially) or are located in ECOLIN_10170. In some embodiments, one or more deletions encompass (completely or partially) or are located in ECOLIN_10175. In some embodiments, one or more deletions encompass (completely or partially) or are located in ECOLIN_10180. In some embodiments, one or more deletions encompass (completely or partially) or are located in ECOLIN_10185. In some embodiments, one or more deletions encompass (completely or partially) or are located in ECOLIN_10190. In some embodiments, one or more deletions encompass (completely or partially) or are located in ECOLIN_10195. In some embodiments, one or more deletions encompass (completely or partially) or are located in ECOLIN_10200. In some embodiments, one or more deletions encompass (completely or partially) or are located in ECOLIN_10205. In some embodiments, one or more deletions encompass (completely or partially) or are located in ECOLIN_10210. In some embodiments, one or more deletions encompass (completely or partially) or are located in ECOLIN_10220. In some embodiments, one or more deletions encompass (completely or partially) or are located in ECOLIN_10225. In some embodiments, one or more deletions encompass (completely or partially) or are located in ECOLIN_10230. In some embodiments, one or more deletions encompass (completely or partially) or are located in ECOLIN_10235. In some embodiments, one or more deletions encompass (completely or partially) or are located in ECOLIN_10240. In some embodiments, one or more deletions encompass (completely or partially) or are located in ECOLIN_10245. In some embodiments, one or more deletions encompass (completely or partially) or are located in ECOLIN_10250. In some embodiments, one or more deletions encompass (completely or partially) or are located in ECOLIN_10255. In some embodiments, one or more deletions encompass (completely or partially) or are located in ECOLIN_10260. In some embodiments, one or more deletions encompass (completely or partially) or are located in ECOLIN_10265. In some embodiments, one or more deletions encompass (completely or partially) or are located in ECOLIN_10270. In some embodiments, one or more deletions encompass (completely or partially) or are located in ECOLIN_10275. In some embodiments, one or more deletions encompass (completely or partially) or are located in ECOLIN_10280. In some embodiments, one or more deletions encompass (completely or partially) or are located in ECOLIN_10290. In some embodiments, one or more deletions encompass (completely or partially) or are located in ECOLIN_10295. In some embodiments, one or more deletions encompass (completely or partially) or are located in ECOLIN_10300. In some embodiments, one or more deletions encompass (completely or partially) or are located in ECOLIN_10305. In some embodiments, one or more deletions encompass (completely or partially) or are located in ECOLIN_10310. In some embodiments, one or more deletions encompass (completely or partially) or are located in ECOLIN_10315. In some embodiments, one or more deletions encompass (completely or partially) or are located in ECOLIN_10320. In some embodiments, one or more deletions encompass (completely or partially) or are located in ECOLIN_10325. In some embodiments, one or more deletions encompass (completely or partially) or are located in ECOLIN_10330. In some embodiments, one or more deletions encompass (completely or partially) or are located in ECOLIN_10335. In some embodiments, one or more deletions encompass (completely or partially) or are located in ECOLIN_10340. In some embodiments, one or more deletions encompass (completely or partially) or are located in ECOLIN_10345.

In some embodiments, the mutations are located in or encompass one or more polypeptides selected from lipid A biosynthesis (KDO)2-(lauroyl)-lipid IVA acyltransferase, peptidase, zinc ABC transporter substrate-binding protein, zinc ABC transporter ATPase, high-affinity zinc transporter membrane component, ATP-dependent DNA helicase RuvB, ATP-dependent DNA helicase RuvA, Holliday junction resolvase, dihydroneopterin triphosphate pyrophosphatase, aspartyl-tRNA synthetase, hydrolase, DNA polymerase V, MsgA, phage tail protein, tail protein, host specificity protein, peptidase P60, tail protein, tail protein, tail fiber protein, Minor tail protein U, DNA breaking-rejoining protein, peptidase S14, capsid protein, DNA packaging protein, terminase, lysozyme, holin, DNA adenine methylase, serine protease, antitermination protein, antirepressor, crossover junction endodeoxyribonuclease, adenine methyltransferase, DNA methyltransferase ECOLIN_10240, GntR family transcriptional regulator ECOLIN_10245, cI repressor, Domain of unknown function (DUF4222); DNA recombinase, Multiple Antibiotic Resistance Regulator (MarR), unknown ead like protein in P22, Protein of unknown function (DUF550); 3′-5′ exonuclease, excisionase, integrase, and tRNA methyltransferase. In one embodiment, one or more of a Minor tail protein U, a tail protein, a DNA breaking-rejoining protein, a peptidase S14, a capsid protein, a DNA packaging protein, and a terminase are deleted.

In one specific embodiment, a Minor tail protein U, a tail protein, a DNA breaking-rejoining protein, a peptidase S14, a capsid protein, a DNA packaging protein, and a terminase are deleted. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Insertions

In some embodiments, the insertion is in a coding region of the E. coli Nissle Phage 3 genome. In some embodiments, the insertion is inserted into a regulatory region of the E. coli Nissle Phage 3 genome. In some embodiments, the inserted polynucleotides comprise one or more antibiotic cassette(s). Suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more inserted polynucleotides comprise at least about 1-500 bp in length. In some embodiments, the one or more inserted polynucleotides comprise at least about 500-1000 bp in length. In some embodiments, the one or more inserted polynucleotides comprise at least about 1000-2000 bp in length. In some embodiments, the one or more inserted polynucleotides comprise at least about 1000-2000 bp in length. In some embodiments, the one or more inserted polynucleotides comprise at least about 2000-3000 bp in length. In some embodiments, the one or more inserted polynucleotides comprise at least about 3000-4000 bp in length. In some embodiments, the one or more inserted polynucleotides comprise at least about 4000-5000 bp in length. In some embodiments, the one or more inserted polynucleotides comprise at least about 5,000-6,000 bp in length. In some embodiments, the one or more inserted polynucleotides comprise at least about 6,000-7,000 bp in length. In some embodiments, the one or more inserted polynucleotides comprise at least about 7,000-8,000 bp in length. In some embodiments, the one or more inserted polynucleotides comprise at least about 8,000-9,000 bp in length. In some embodiments, the one or more inserted polynucleotides comprise at least about 9,000-10,000 bp in length. In some embodiments, the one or more inserted polynucleotides comprise at least about 10,000-15,000 bp in length. In some embodiments, the one or more inserted polynucleotides comprise at least about 10,000-15,000 bp in length, at least about 15,000-20,000 bp in length, at least about 20,000-25,000 bp in length, at least about 25,000-30,000 bp in length, at least about 30,000-35,000 bp in length, at least about 35,000-40,000 bp in length, at least about 40,000-45,000 bp in length, at least about 45,000-50,000 bp in length, at least about 50,000-55,000 bp in length, at least about 55,000-60,000 bp in length, at least about 60,000-65,000 bp in length, at least about 65,000-70,000 bp in length, at least about 70,000-75,000 bp in length, at least about 75,000-80,000 bp in length, at least about 80,000-85,000 bp in length, at least about 85,000-90,000 bp in length, at least about 90,000-95,000 bp in length, 95,000-100,000 bp in length, at least about 100,000-110,000 bp in length, at least about 110,000-120,000 bp in length, at least about 120,000-130,000 bp in length, at least about 130,000-140,000 bp in length, at least about 140,000-150,000 bp in length, at least about 150,000-200,000 bp in length, or more than at least about 200,000 bp in length. In one specific embodiment, at least about 9687 bp in length are inserted.

In some embodiments, the one or more insertions are located within at least about 1-500 bp of the E. coli Nissle Phage 3 genome. In some embodiments, the one or more insertions are located within at least about 500-1000 bp of the E. coli Nissle Phage 3 genome. In some embodiments, the one or more insertions are located within at least about 1000-2000 bp of the E. coli Nissle Phage 3 genome. In some embodiments, the one or more insertions are located within at least about 1000-2000 bp of the E. coli Nissle Phage 3 genome. In some embodiments, the one or more insertions are located within at least about 2000-3000 bp of the E. coli Nissle Phage 3 genome. In some embodiments, the one or more insertions are located within at least about 3000-4000 bp of the E. coli Nissle Phage 3 genome. In some embodiments, the one or more insertions are located within at least about 4000-5000 bp of the E. coli Nissle Phage 3 genome. In some embodiments, the one or more insertions are located within at least about 5,000-6,000 bp of the E. coli Nissle Phage 3 genome. In some embodiments, the one or more insertions are located within at least about 6,000-7,000 bp of the E. coli Nissle Phage 3 genome. In some embodiments, the one or more insertions are located within at least about 7,000-8,000 bp of the E. coli Nissle Phage 3 genome. In some embodiments, the one or more insertions are located within at least about 8,000-9,000 bp of the E. coli Nissle Phage 3 genome. In some embodiments, the one or more insertions are located within at least about 9,000-10,000 bp of the E. coli Nissle Phage 3 genome. In some embodiments, the one or more insertions are located within at least about 10,000-15,000 bp of the E. coli Nissle Phage 3 genome. In some embodiments, the one or more insertions are located within at least about 10,000-15,000 bp of the E. coli Nissle Phage 3 genome, at least about 15,000-20,000 bp of the E. coli Nissle Phage 3 genome, at least about 20,000-25,000 bp of the E. coli Nissle Phage 3 genome, at least about 25,000-30,000 bp of the E. coli Nissle Phage 3 genome, at least about 30,000-35,000 bp of the E. coli Nissle Phage 3 genome, at least about 35,000-40,000 bp of the E. coli Nissle Phage 3 genome, at least about 40,000-45,000 bp of the E. coli Nissle Phage 3 genome, at least about 45,000-50,000 bp of the E. coli Nissle Phage 3 genome, at least about 50,000-55,000 bp of the E. coli Nissle Phage 3 genome, or at least about 55,000-60,000 bp of the E. coli Nissle Phage 3 genome. In one specific embodiment, 9687 bp of the E. coli Nissle Phage 3 genome are inserted. In some embodiments, the inserted nucleotides are interspersed. In some embodiments, the inserted nucleotides are consecutive.

In some embodiments, the insertions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the E. coli Nissle Phage 3 genome. In some embodiments, at least about 30-40% of the E. coli Nissle Phage 3 genome is inserted. In some embodiments, the insertions are located within at least about 40-50% of the E. coli Nissle Phage 3 genome. In some embodiments, the insertions are located within at least about 50-60% of the E. coli Nissle Phage 3 genome. In some embodiments, the insertions are located within at least about 60-70% of the E. coli Nissle Phage 3 genome. In some embodiments, the insertions are located within at least about 70-80% of the E. coli Nissle Phage 3 genome. In some embodiments, the insertions are located within at least about 80-90% of the E. coli Nissle Phage 3 genome. In some embodiments, the insertions are located within at least about 90-100% of the E. coli Nissle Phage 3 genome.

In some embodiments, at least about 1, 2, 3, 4, 5, 6, 7, 8, 9, 10, 11, 12, 13, 14, 15, 16, 17, 18, 19, or 20 genes comprise insertions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise insertions. In some embodiments, 13 genes comprise insertions. In one embodiment, 74 genes comprise insertions.

In some embodiments, the one or more insertions are located at the beginning or 5′ end of the E. coli Nissle Phage 3 genome. In some embodiments, the one or more insertions are located at the end or 3′ end of the E. coli Nissle Phage 3 genome. In some embodiments, the one or more insertions are located in the middle of the E. coli Nissle Phage 3 genome. In some embodiments, the E. coli Nissle Phage 3 genes are interspersed within the bacterial genome and the insertion are located in one or more of the interspersed positions.

In some embodiments, the region for an optimal insertion, i.e., to achieve a desired effect, can be determined through analysis of homology with other phages in other bacteria. Homologous conserved regions in phages may be suitable for insertion, as these are conserved and may comprise one or more essential genes. In some embodiments, regulatory elements, such as promoters, are inserted. In some embodiments, coding sequences are inserted. In some embodiments, the one or more inserted regions contain one or more genes essential for the lytic cycle.

In some embodiments, the insertions are located within one or more genes encoding lytic genes. In some embodiments, the insertions are located within one or more genes encoding one or more proteases or lysins. In some embodiments, the insertions are located within one or more genes encoding one or more toxins. In some embodiments, the insertions are located within one or more genes encoding one or more antibiotic resistance related proteins. In some embodiments, the insertions are located within one or more genes encoding one or phage translation related proteins. In some embodiments, the one or more insertions are located within one or more genes encoding structural proteins. Such structural genes include genes encoding polypeptides of the head, tail, collar, or coat. In some embodiments, the one or more mutations are located within or encompass one or more genes encoding head proteins. In some embodiments, the one or more mutations are located within or encompass one or more genes encoding tail proteins. In some embodiments, the one or more mutations are located within or encompass one or more genes encoding collar proteins. In some embodiments, the one or more mutations are located within or encompass one or more genes encoding coat proteins. In some embodiments, the insertions are located within one or more genes encoding one or more plate proteins. In some embodiments, the insertions are located within one or more genes encoding one or more proteins require for assembly of the bacteriophage. In some embodiments, the insertions are located within one or more genes encoding one or more portal proteins. In some embodiments, the insertions are located within one or more genes encoding one or more polypeptides involved in recombination. In some embodiments, the insertions are located within one or more genes encoding one or more integrases. In some embodiments, the insertions are located within one or more genes encoding one or more invertases. In some embodiments, the insertions are located within one or more genes encoding one or more transposases. In some embodiments, the insertions are located with within one or more genes encoding one or more polypeptides involved in replication or translation. In some embodiments, the insertions are located within one or more genes encoding one or more primases. In some embodiments, the insertions are located within one or more genes encoding one or more tRNA related proteins. In some embodiments, the insertions are located within one or more genes encoding one or more polypeptides involved in phage insertion. In some embodiments, the insertions are located within one or more genes encoding an attachment site. In some embodiments, the insertions are located within one or more genes encoding one or more polypeptides involved in packaging. In some embodiments, the insertions are located within one or more genes encoding one or more terminases. In some embodiments, the insertions are located within one or more genes encoding one or more host genes.

In some embodiments, the insertions are located within genes encoding one or more polypeptides involved in one or more of cell lysis, phage structure, phage assembly, phage packaging recombination, replication or translation, phage insertion, or are host proteins, and combinations thereof.

In some embodiments, the insertions are located within genes encoding one or more polypeptides involved in one or more of cell lysis, phage structure, phage assembly, phage packaging recombination, replication or translation, phage insertion, and combinations thereof.

In some embodiments, the insertions are located within 1 genes encoding polypeptides involved in cell lysis, phage structure, phage assembly, phage packaging recombination, replication or translation, phage insertion, and combinations thereof. In some embodiments, the insertions are located within 2 genes encoding polypeptides involved in cell lysis, phage structure, phage assembly, phage packaging recombination, replication or translation, phage insertion, and combinations thereof. In some embodiments, the insertions are located within 3 genes encoding polypeptides involved in cell lysis, phage structure, phage assembly, phage packaging recombination, replication or translation, phage insertion, and combinations thereof. In some embodiments, the insertions are located within 4 genes encoding polypeptides involved in cell lysis, phage structure, phage assembly, phage packaging recombination, replication or translation, phage insertion, and combinations thereof. In some embodiments, the insertions are located within 2 genes encoding polypeptides involved in cell lysis, phage structure, phage assembly, phage packaging recombination, replication or translation, phage insertion, and combinations thereof. In some embodiments, the insertions are located within 5 genes encoding polypeptides involved in cell lysis, phage structure, phage assembly, phage packaging recombination, replication or translation, phage insertion, and combinations thereof. In some embodiments, the insertions are located within 6 genes encoding polypeptides involved in cell lysis, phage structure, phage assembly, phage packaging recombination, replication or translation, phage insertion, and combinations thereof. In some embodiments, the insertions are located within 7 genes encoding polypeptides involved in cell lysis, phage structure, phage assembly, phage packaging recombination, replication or translation, phage insertion, and combinations thereof. In some embodiments, the insertions are located within 8 genes encoding polypeptides involved in cell lysis, phage structure, phage assembly, phage packaging recombination, replication or translation, phage insertion, and combinations thereof. In some embodiments, the insertions are located within 9 genes encoding polypeptides involved in cell lysis, phage structure, phage assembly, phage packaging recombination, replication or translation, phage insertion, and combinations thereof. In some embodiments, the insertions are located within 10 genes encoding polypeptides involved in cell lysis, phage structure, phage assembly, phage packaging recombination, replication or translation, phage insertion, and combinations thereof. In some embodiments, the insertions are located within 11 genes encoding polypeptides involved in cell lysis, phage structure, phage assembly, phage packaging recombination, replication or translation, phage insertion, and combinations thereof. In some embodiments, the insertions are located within 12 genes encoding polypeptides involved in cell lysis, phage structure, phage assembly, phage packaging recombination, replication or translation, phage insertion, and combinations thereof. In some embodiments, the insertions are located within 13 genes encoding polypeptides involved in cell lysis, phage structure, phage assembly, phage packaging recombination, replication or translation, phage insertion, and combinations thereof. In some embodiments, the insertions are located within 14 genes encoding polypeptides involved in cell lysis, phage structure, phage assembly, phage packaging recombination, replication or translation, phage insertion, and combinations thereof. In some embodiments, the insertions are located within 15 genes encoding polypeptides involved in cell lysis, phage structure, phage assembly, phage packaging recombination, replication or translation, phage insertion, and combinations thereof. In some embodiments, the insertions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage insertion, and combinations thereof. In some embodiments, the insertions are located within one or more host proteins within the phage genome.

In any of the embodiments described herein, the insertions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In some embodiments, one or more insertions are located in ECOLIN_09965. In some embodiments, one or more insertions are located in ECOLIN_09970. In some embodiments, one or more insertions are located in ECOLIN_09975. In some embodiments, one or more insertions are located in ECOLIN_09980. In some embodiments, one or more insertions are located in ECOLIN_09985. In some embodiments, one or more insertions are located in ECOLIN_09990. In some embodiments, one or more insertions are located in ECOLIN_09995. In some embodiments, one or more insertions are located in ECOLIN_10000. In some embodiments, one or more insertions are located in ECOLIN_10005. In some embodiments, one or more insertions are located in ECOLIN_10010. In some embodiments, one or more insertions are located in ECOLIN_10015. In some embodiments, one or more insertions are located in ECOLIN_10020. In some embodiments, one or more insertions are located in ECOLIN_10025. In some embodiments, one or more insertions are located in ECOLIN_10030. In some embodiments, one or more insertions are located in ECOLIN_10035. In some embodiments, one or more insertions are located in ECOLIN_10040. In some embodiments, one or more insertions are located in ECOLIN_10045. In some embodiments, one or more insertions are located in ECOLIN_10050. In some embodiments, one or more insertions are located in ECOLIN_10055. In some embodiments, one or more insertions are located in ECOLIN_10065. In some embodiments, one or more insertions are located in ECOLIN_10070. In some embodiments, one or more insertions are located in ECOLIN_10075. In some embodiments, one or more insertions are located in ECOLIN_10080. In some embodiments, one or more insertions are located in ECOLIN_10085. In some embodiments, one or more insertions are located in ECOLIN_10090. In some embodiments, one or more insertions are located in ECOLIN_10095. In some embodiments, one or more insertions are located in ECOLIN_10100. In some embodiments, one or more insertions are located in ECOLIN_10105. In some embodiments, one or more insertions are located in ECOLIN_10110. In some embodiments, one or more insertions are located in ECOLIN_10115. In some embodiments, one or more insertions are located in ECOLIN_10120. In some embodiments, one or more insertions are located in ECOLIN_10125. In some embodiments, one or more insertions are located in ECOLIN_10130. In some embodiments, one or more insertions are located in ECOLIN_10135. In some embodiments, one or more insertions are located in ECOLIN_10140. In some embodiments, one or more insertions are located in ECOLIN_10145. In some embodiments, one or more insertions are located in ECOLIN_10150. In some embodiments, one or more insertions are located in ECOLIN_10160. In some embodiments, one or more insertions are located in ECOLIN_10165. In some embodiments, one or more insertions are located in ECOLIN_10170. In some embodiments, one or more insertions are located in ECOLIN_10175. In some embodiments, one or more insertions are located in ECOLIN_10180. In some embodiments, one or more insertions are located in ECOLIN_10185. In some embodiments, one or more insertions are located in ECOLIN_10190. In some embodiments, one or more insertions are located in ECOLIN_10195. In some embodiments, one or more insertions are located in ECOLIN_10200. In some embodiments, one or more insertions are located in ECOLIN_10205. In some embodiments, one or more insertions are located in ECOLIN_10210. In some embodiments, one or more insertions are located in ECOLIN_10220. In some embodiments, one or more insertions are located in ECOLIN_10225. In some embodiments, one or more insertions are located in ECOLIN_10230. In some embodiments, one or more insertions are located in ECOLIN_10235. In some embodiments, one or more insertions are located in ECOLIN_10240. In some embodiments, one or more insertions are located in ECOLIN_10245. In some embodiments, one or more insertions are located in ECOLIN_10250. In some embodiments, one or more insertions are located in ECOLIN_10255. In some embodiments, one or more insertions are located in ECOLIN_10260. In some embodiments, one or more insertions are located in ECOLIN_10265. In some embodiments, one or more insertions are located in ECOLIN_10270. In some embodiments, one or more insertions are located in ECOLIN_10275. In some embodiments, one or more insertions are located in ECOLIN_10280. In some embodiments, one or more insertions are located in ECOLIN_10290. In some embodiments, one or more insertions are located in ECOLIN_10295. In some embodiments, one or more insertions are located in ECOLIN_10300. In some embodiments, one or more insertions are located in ECOLIN_10305. In some embodiments, one or more insertions are located in ECOLIN_10310. In some embodiments, one or more insertions are located in ECOLIN_10315. In some embodiments, one or more insertions are located in ECOLIN_10320. In some embodiments, one or more insertions are located in ECOLIN_10325. In some embodiments, one or more insertions are located in ECOLIN_10330. In some embodiments, one or more insertions are located in ECOLIN_10335. In some embodiments, one or more insertions are located in ECOLIN_10340. In some embodiments, one or more insertions are located in ECOLIN_10345.

In some embodiments, the mutations are located in or encompass one or more polypeptides selected from lipid A biosynthesis (KDO)2-(lauroyl)-lipid IVA acyltransferase, peptidase, zinc ABC transporter substrate-binding protein, zinc ABC transporter ATPase, high-affinity zinc transporter membrane component, ATP-dependent DNA helicase RuvB, ATP-dependent DNA helicase RuvA, Holliday junction resolvase, dihydroneopterin triphosphate pyrophosphatase, aspartyl-tRNA synthetase, hydrolase, DNA polymerase V, MsgA, phage tail protein, tail protein, host specificity protein, peptidase P60, tail protein, tail protein, tail fiber protein, Minor tail protein U, DNA breaking-rejoining protein, peptidase S14, capsid protein, DNA packaging protein, terminase, lysozyme, holin, DNA adenine methylase, serine protease, antitermination protein, antirepressor, crossover junction endodeoxyribonuclease, adenine methyltransferase, DNA methyltransferase ECOLIN_10240, GntR family transcriptional regulator ECOLIN_10245, cI repressor, Domain of unknown function (DUF4222); DNA recombinase, Multiple Antibiotic Resistance Regulator (MarR), unknown ead like protein in P22, Protein of unknown function (DUF550); 3′-5′ exonuclease, excisionase, integrase, and tRNA methyltransferase. In one embodiment, one or more of a Minor tail protein U, a tail protein, a DNA breaking-rejoining protein, a peptidase S14, a capsid protein, a DNA packaging protein, and a terminase contain one or more insertions. In one specific embodiment, a Minor tail protein U, a tail protein, a DNA breaking-rejoining protein, a peptidase S14, a capsid protein, a DNA packaging protein, and a terminase contain one or more insertions.

In one embodiment one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175 comprise an insertion.

### Inversions

In some embodiments, the inversion is in a coding region of the E. coli Nissle Phage 3 genome. In some embodiments, the inversion is inverted into a regulatory region of the E. coli Nissle Phage 3 genome. In some embodiments, the inversions comprise one or more antibiotic cassette(s). suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more inversions comprise 1-500 bp. In some embodiments, the one or more inversions comprise at least about 500-1000 bp. In some embodiments, the one or more inversions comprise at least about 1000-2000 bp. In some embodiments, the one or more inversions comprise at least about 1000-2000 bp. In some embodiments, the one or more inversions comprise at least about 2000-3000 bp. In some embodiments, the one or more inversions comprise at least about 3000-4000 bp. In some embodiments, the one or more inversions comprise at least about 4000-5000 bp. In some embodiments, the one or more inversions comprise at least about 5,000-6,000 bp. In some embodiments, the one or more inversions comprise at least about 6,000-7,000 bp. In some embodiments, the one or more inversions comprise at least about 7,000-8,000 bp. In some embodiments, the one or more inversions comprise at least about 8,000-9,000 bp. In some embodiments, the one or more inversions comprise at least about 9,000-10,000 bp. In some embodiments, the one or more inversions comprise at least about 10,000-15,000 bp. In some embodiments, the one or more inversions comprise at least about 10,000-15,000 bp, at least about 15,000-20,000 bp, at least about 20,000-25,000 bp, at least about 25,000-30,000 bp, at least about 30,000-35,000 bp, at least about 35,000-40,000 bp, at least about 40,000-45,000 bp, at least about 45,000-50,000 bp, at least about 50,000-55,000 bp, or at least about 55,000-60,000 bp. In one specific embodiment, 9687 bp are inverted. In some embodiments, the inverted nucleotides are interspersed. In some embodiments, the inverted nucleotides are consecutive.

In some embodiments, the one or more inversions are located within at least about 1-500 bp of the E. coli Nissle Phage 3 genome. In some embodiments, the one or more inversions are located within at least about 500-1000 bp of the E. coli Nissle Phage 3 genome. In some embodiments, the one or more inversions are located within at least about 1000-2000 bp of the E. coli Nissle Phage 3 genome. In some embodiments, the one or more inversions are located within at least about 1000-2000 bp of the E. coli Nissle Phage 3 genome. In some embodiments, the one or more inversions are located within at least about 2000-3000 bp of the E. coli Nissle Phage 3 genome. In some embodiments, the one or more inversions are located within at least about 3000-4000 bp of the E. coli Nissle Phage 3 genome. In some embodiments, the one or more inversions are located within at least about 4000-5000 bp of the E. coli Nissle Phage 3 genome. In some embodiments, the one or more inversions are located within at least about 5,000-6,000 bp of the E. coli Nissle Phage 3 genome. In some embodiments, the one or more inversions are located within at least about 6,000-7,000 bp of the E. coli Nissle Phage 3 genome. In some embodiments, the one or more inversions are located within at least about 7,000-8,000 bp of the E. coli Nissle Phage 3 genome. In some embodiments, the one or more inversions are located within at least about 8,000-9,000 bp of the E. coli Nissle Phage 3 genome. In some embodiments, the one or more inversions are located within at least about 9,000-10,000 bp of the E. coli Nissle Phage 3 genome. In some embodiments, the one or more inversions are located within at least about 10,000-15,000 bp of the E. coli Nissle Phage 3 genome. In some embodiments, the one or more inversions are located within at least about 10,000-15,000 bp of the E. coli Nissle Phage 3 genome, at least about 15,000-20,000 bp of the E. coli Nissle Phage 3 genome, at least about 20,000-25,000 bp of the E. coli Nissle Phage 3 genome, at least about 25,000-30,000 bp of the E. coli Nissle Phage 3 genome, at least about 30,000-35,000 bp of the E. coli Nissle Phage 3 genome, at least about 35,000-40,000 bp of the E. coli Nissle Phage 3 genome, at least about 40,000-45,000 bp of the E. coli Nissle Phage 3 genome, at least about 45,000-50,000 bp of the E. coli Nissle Phage 3 genome, at least about 50,000-55,000 bp of the E. coli Nissle Phage 3 genome, or at least about 55,000-60,000 bp of the E. coli Nissle Phage 3 genome. In some embodiments, the inverted nucleotides are interspersed. In some embodiments, the inverted nucleotides are consecutive.

In some embodiments, the inversions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the E. coli Nissle Phage 3 genome. In some embodiments, at least about 30-40% of the E. coli Nissle Phage 3 genome is inverted. In some embodiments, the inversions are located within at least about 40-50% of the E. coli Nissle Phage 3 genome. In some embodiments, the inversions are located within at least about 50-60% of the E. coli Nissle Phage 3 genome. In some embodiments, the inversions are located within at least about 60-70% of the E. coli Nissle Phage 3 genome. In some embodiments, the inversions are located within at least about 70-80% of the E. coli Nissle Phage 3 genome. In some embodiments, the inversions are located within at least about 80-90% of the E. coli Nissle Phage 3 genome. In some embodiments, the inversions are located within at least about 90-100% of the E. coli Nissle Phage 3 genome.

In some embodiments, at least about 1, 2, 3, 4, 5, 6, 7, 8, 9, 10, 11, 12, 13, 14, 15, 16, 17, 18, 19, or 20 genes comprise inversions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise inversions. In some embodiments, 13 genes comprise inversions. In one embodiment, 74 genes comprise inversions.

In some embodiments, the one or more inversions are located at the beginning or 5′ end of the E. coli Nissle Phage 3 genome. In some embodiments, the one or more inversions are located at the end or 3′ end of the E. coli Nissle Phage 3 genome. In some embodiments, the one or more inversions are located in the middle of the E. coli Nissle Phage 3 genome. In some embodiments, the E. coli Nissle Phage 3 genes are interspersed within the bacterial genome and the inversion are located in one or more of the interspersed positions.

In some embodiments, the region for an optimal inversion, i.e., to achieve a desired effect, can be determined through analysis of homology with other phages in other bacteria. Homologous conserved regions in phages may be suitable for inversion, as these are conserved and may comprise one or more essential genes. In some embodiments, regulatory elements, such as promoters, are inverted. In some embodiments, coding sequences are inverted. In some embodiments, the one or more inverted regions contain one or more genes essential for the lytic cycle.

In some embodiments, the inversions are located within one or more genes encoding lytic genes. In some embodiments, the inversions are located within one or more genes encoding one or more proteases or lysins. In some embodiments, the inversions are located within one or more genes encoding one or more toxins. In some embodiments, the inversions are located within one or more genes encoding one or more antibiotic resistance related proteins. In some embodiments, the inversions are located within one or more genes encoding one or phage translation related proteins. In some embodiments, the one or more inversions are located within one or more genes encoding structural proteins. Such structural genes include genes encoding polypeptides of the head, tail, collar, or coat. In some embodiments, the one or more mutations are located within or encompass one or more genes encoding head proteins. In some embodiments, the one or more mutations are located within or encompass one or more genes encoding tail proteins. In some embodiments, the one or more mutations are located within or encompass one or more genes encoding collar proteins. In some embodiments, the one or more mutations are located within or encompass one or more genes encoding coat proteins. In some embodiments, the inversions are located within one or more genes encoding one or more plate proteins. In some embodiments, the inversions are located within one or more genes encoding one or more proteins require for assembly of the bacteriophage. In some embodiments, the inversions are located within one or more genes encoding one or more portal proteins. In some embodiments, the inversions are located within one or more genes encoding one or more polypeptides involved in recombination. In some embodiments, the inversions are located within one or more genes encoding one or more integrases. In some embodiments, the inversions are located within one or more genes encoding one or more invertases. In some embodiments, the inversions are located within one or more genes encoding one or more transposases. In some embodiments, the inversions are located with within one or more genes encoding one or more polypeptides involved in replication or translation. In some embodiments, the inversions are located within one or more genes encoding one or more primases. In some embodiments, the inversions are located within one or more genes encoding one or more tRNA related proteins. In some embodiments, the inversions are located within one or more genes encoding one or more polypeptides involved in phage inversion. In some embodiments, the inversions are located within one or more genes encoding an attachment site. In some embodiments, the inversions are located within one or more genes encoding one or more polypeptides involved in packaging. In some embodiments, the inversions are located within one or more genes encoding one or more terminases. In some embodiments, the inversions are located within one or more genes encoding one or more host genes.

In some embodiments, the inversions are located within genes encoding one or more polypeptides involved in one or more of cell lysis, phage structure, phage assembly, phage packaging recombination, replication or translation, phage inversion, or are host proteins, and combinations thereof.

In some embodiments, the inversions are located within genes encoding one or more polypeptides involved in one or more of cell lysis, phage structure, phage assembly, phage packaging recombination, replication or translation, phage inversion, and combinations thereof.

In some embodiments, the inversions are located within 1 genes encoding polypeptides involved in cell lysis, phage structure, phage assembly, phage packaging recombination, replication or translation, phage inversion, and combinations thereof. In some embodiments, the inversions are located within 2 genes encoding polypeptides involved in cell lysis, phage structure, phage assembly, phage packaging recombination, replication or translation, phage inversion, and combinations thereof. In some embodiments, the inversions are located within 3 genes encoding polypeptides involved in cell lysis, phage structure, phage assembly, phage packaging recombination, replication or translation, phage inversion, and combinations thereof. In some embodiments, the inversions are located within 4 genes encoding polypeptides involved in cell lysis, phage structure, phage assembly, phage packaging recombination, replication or translation, phage inversion, and combinations thereof. In some embodiments, the inversions are located within 2 genes encoding polypeptides involved in cell lysis, phage structure, phage assembly, phage packaging recombination, replication or translation, phage inversion, and combinations thereof. In some embodiments, the inversions are located within 5 genes encoding polypeptides involved in cell lysis, phage structure, phage assembly, phage packaging recombination, replication or translation, phage inversion, and combinations thereof. In some embodiments, the inversions are located within 6 genes encoding polypeptides involved in cell lysis, phage structure, phage assembly, phage packaging recombination, replication or translation, phage inversion, and combinations thereof. In some embodiments, the inversions are located within 7 genes encoding polypeptides involved in cell lysis, phage structure, phage assembly, phage packaging recombination, replication or translation, phage inversion, and combinations thereof. In some embodiments, the inversions are located within 8 genes encoding polypeptides involved in cell lysis, phage structure, phage assembly, phage packaging recombination, replication or translation, phage inversion, and combinations thereof. In some embodiments, the inversions are located within 9 genes encoding polypeptides involved in cell lysis, phage structure, phage assembly, phage packaging recombination, replication or translation, phage inversion, and combinations thereof. In some embodiments, the inversions are located within 10 genes encoding polypeptides involved in cell lysis, phage structure, phage assembly, phage packaging recombination, replication or translation, phage inversion, and combinations thereof. In some embodiments, the inversions are located within 11 genes encoding polypeptides involved in cell lysis, phage structure, phage assembly, phage packaging recombination, replication or translation, phage inversion, and combinations thereof. In some embodiments, the inversions are located within 12 genes encoding polypeptides involved in cell lysis, phage structure, phage assembly, phage packaging recombination, replication or translation, phage inversion, and combinations thereof. In some embodiments, the inversions are located within 13 genes encoding polypeptides involved in cell lysis, phage structure, phage assembly, phage packaging recombination, replication or translation, phage inversion, and combinations thereof. In some embodiments, the inversions are located within 14 genes encoding polypeptides involved in cell lysis, phage structure, phage assembly, phage packaging recombination, replication or translation, phage inversion, and combinations thereof. In some embodiments, the inversions are located within 15 genes encoding polypeptides involved in cell lysis, phage structure, phage assembly, phage packaging recombination, replication or translation, phage inversion, and combinations thereof. In some embodiments, the inversions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage inversion, and combinations thereof. In some embodiments, the inversions are located within one or more host proteins within the phage genome.

In any of the embodiments described herein, the inversions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In some embodiments, one or more inversions encompass (completely or partially) or are located in ECOLIN_09965. In some embodiments, one or more inversions encompass (completely or partially) or are located in ECOLIN_09970. In some embodiments, one or more inversions encompass (completely or partially) or are located in ECOLIN_09975. In some embodiments, one or more inversions encompass (completely or partially) or are located in ECOLIN_09980. In some embodiments, one or more inversions encompass (completely or partially) or are located in ECOLIN_09985. In some embodiments, one or more inversions encompass (completely or partially) or are located in ECOLIN_09990. In some embodiments, one or more inversions encompass (completely or partially) or are located in ECOLIN_09995. In some embodiments, one or more inversions encompass (completely or partially) or are located in ECOLIN_10000. In some embodiments, one or more inversions encompass (completely or partially) or are located in ECOLIN_10005. In some embodiments, one or more inversions encompass (completely or partially) or are located in ECOLIN_10010. In some embodiments, one or more inversions encompass (completely or partially) or are located in ECOLIN_10015. In some embodiments, one or more inversions encompass (completely or partially) or are located in ECOLIN_10020. In some embodiments, one or more inversions encompass (completely or partially) or are located in ECOLIN_10025. In some embodiments, one or more inversions encompass (completely or partially) or are located in ECOLIN_10030. In some embodiments, one or more inversions encompass (completely or partially) or are located in ECOLIN_10035. In some embodiments, one or more inversions encompass (completely or partially) or are located in ECOLIN_10040. In some embodiments, one or more inversions encompass (completely or partially) or are located in ECOLIN_10045. In some embodiments, one or more inversions encompass (completely or partially) or are located in ECOLIN_10050. In some embodiments, one or more inversions encompass (completely or partially) or are located in ECOLIN_10055. In some embodiments, one or more inversions encompass (completely or partially) or are located in ECOLIN_10065. In some embodiments, one or more inversions encompass (completely or partially) or are located in ECOLIN_10070. In some embodiments, one or more inversions encompass (completely or partially) or are located in ECOLIN_10075. In some embodiments, one or more inversions encompass (completely or partially) or are located in ECOLIN_10080. In some embodiments, one or more inversions encompass (completely or partially) or are located in ECOLIN_10085. In some embodiments, one or more inversions encompass (completely or partially) or are located in ECOLIN_10090. In some embodiments, one or more inversions encompass (completely or partially) or are located in ECOLIN_10095. In some embodiments, one or more inversions encompass (completely or partially) or are located in ECOLIN_10100. In some embodiments, one or more inversions encompass (completely or partially) or are located in ECOLIN_10105. In some embodiments, one or more inversions encompass (completely or partially) or are located in ECOLIN_10110. In some embodiments, one or more inversions encompass (completely or partially) or are located in ECOLIN_10115. In some embodiments, one or more inversions encompass (completely or partially) or are located in ECOLIN_10120. In some embodiments, one or more inversions encompass (completely or partially) or are located in ECOLIN_10125. In some embodiments, one or more inversions encompass (completely or partially) or are located in ECOLIN_10130. In some embodiments, one or more inversions encompass (completely or partially) or are located in ECOLIN_10135. In some embodiments, one or more inversions encompass (completely or partially) or are located in ECOLIN_10140. In some embodiments, one or more inversions encompass (completely or partially) or are located in ECOLIN_10145. In some embodiments, one or more inversions encompass (completely or partially) or are located in ECOLIN_10150. In some embodiments, one or more inversions encompass (completely or partially) or are located in ECOLIN_10160. In some embodiments, one or more inversions encompass (completely or partially) or are located in ECOLIN_10165. In some embodiments, one or more inversions encompass (completely or partially) or are located in ECOLIN_10170. In some embodiments, one or more inversions encompass (completely or partially) or are located in ECOLIN_10175. In some embodiments, one or more inversions encompass (completely or partially) or are located in ECOLIN_10180. In some embodiments, one or more inversions encompass (completely or partially) or are located in ECOLIN_10185. In some embodiments, one or more inversions encompass (completely or partially) or are located in ECOLIN_10190. In some embodiments, one or more inversions encompass (completely or partially) or are located in ECOLIN_10195. In some embodiments, one or more inversions encompass (completely or partially) or are located in ECOLIN_10200. In some embodiments, one or more inversions encompass (completely or partially) or are located in ECOLIN_10205. In some embodiments, one or more inversions encompass (completely or partially) or are located in ECOLIN_10210. In some embodiments, one or more inversions encompass (completely or partially) or are located in ECOLIN_10220. In some embodiments, one or more inversions encompass (completely or partially) or are located in ECOLIN_10225. In some embodiments, one or more inversions encompass (completely or partially) or are located in ECOLIN_10230. In some embodiments, one or more inversions encompass (completely or partially) or are located in ECOLIN_10235. In some embodiments, one or more inversions encompass (completely or partially) or are located in ECOLIN_10240. In some embodiments, one or more inversions encompass (completely or partially) or are located in ECOLIN_10245. In some embodiments, one or more inversions encompass (completely or partially) or are located in ECOLIN_10250. In some embodiments, one or more inversions encompass (completely or partially) or are located in ECOLIN_10255. In some embodiments, one or more inversions encompass (completely or partially) or are located in ECOLIN_10260. In some embodiments, one or more inversions encompass (completely or partially) or are located in ECOLIN_10265. In some embodiments, one or more inversions encompass (completely or partially) or are located in ECOLIN_10270. In some embodiments, one or more inversions encompass (completely or partially) or are located in ECOLIN_10275. In some embodiments, one or more inversions encompass (completely or partially) or are located in ECOLIN_10280. In some embodiments, one or more inversions encompass (completely or partially) or are located in ECOLIN_10290. In some embodiments, one or more inversions encompass (completely or partially) or are located in ECOLIN_10295. In some embodiments, one or more inversions encompass (completely or partially) or are located in ECOLIN_10300. In some embodiments, one or more inversions encompass (completely or partially) or are located in ECOLIN_10305. In some embodiments, one or more inversions encompass (completely or partially) or are located in ECOLIN_10310. In some embodiments, one or more inversions encompass (completely or partially) or are located in ECOLIN_10315. In some embodiments, one or more inversions encompass (completely or partially) or are located in ECOLIN_10320. In some embodiments, one or more inversions encompass (completely or partially) or are located in ECOLIN_10325. In some embodiments, one or more inversions encompass (completely or partially) or are located in ECOLIN_10330. In some embodiments, one or more inversions encompass (completely or partially) or are located in ECOLIN_10335. In some embodiments, one or more inversions encompass (completely or partially) or are located in ECOLIN_10340. In some embodiments, one or more inversions encompass (completely or partially) or are located in ECOLIN_10345.

In some embodiments, the mutations are located in or encompass one or more polypeptides selected from lipid A biosynthesis (KDO)2-(lauroyl)-lipid IVA acyltransferase, peptidase, zinc ABC transporter substrate-binding protein, zinc ABC transporter ATPase, high-affinity zinc transporter membrane component, ATP-dependent DNA helicase RuvB, ATP-dependent DNA helicase RuvA, Holliday junction resolvase, dihydroneopterin triphosphate pyrophosphatase, aspartyl-tRNA synthetase, hydrolase, DNA polymerase V, MsgA, phage tail protein, tail protein, host specificity protein, peptidase P60, tail protein, tail protein, tail fiber protein, Minor tail protein U, DNA breaking-rejoining protein, peptidase S14, capsid protein, DNA packaging protein, terminase, lysozyme, holin, DNA adenine methylase, serine protease, antitermination protein, antirepressor, crossover junction endodeoxyribonuclease, adenine methyltransferase, DNA methyltransferase ECOLIN_10240, GntR family transcriptional regulator ECOLIN_10245, cI repressor, Domain of unknown function (DUF4222); DNA recombinase, Multiple Antibiotic Resistance Regulator (MarR), unknown ead like protein in P22, Protein of unknown function (DUF550); 3′-5′ exonuclease, excisionase, integrase, and tRNA methyltransferase. In one embodiment, one or more of a Minor tail protein U, a tail protein, a DNA breaking-rejoining protein, a peptidase S14, a capsid protein, a DNA packaging protein, and a terminase are inverted. In one specific embodiment, a Minor tail protein U, a tail protein, a DNA breaking-rejoining protein, a peptidase S14, a capsid protein, a DNA packaging protein, and a terminase are inverted.

In one embodiment, the inversion is a complete or partial inversion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the inversion is a complete or partial inversion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the inversion is a complete inversion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial inversion of ECOLIN_10175. In one embodiment, the sequence of SEQ ID NO: 130 is inverted from the Phage 3 genome. In one embodiment, a sequence comprising SEQ ID NO: 130 is inverted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Substitutions

In some embodiments, the substitution is in a coding region of the E. coli Nissle Phage 3 genome. In some embodiments, the substitution is substituted into a regulatory region of the E. coli Nissle Phage 3 genome. In some embodiments, the substitutions comprise one or more antibiotic cassette(s). suitable antibiotic cassettes are known in the art, and non-limiting examples of such antibiotic cassettes are described herein. In some embodiments, the antibiotic is chloramphenicol. In some embodiments, the antibiotic is kanamycin. In some embodiments, the antibiotic is ampicillin. In some embodiments, the antibiotic is chloramphenicol and kanamycin. In some embodiments, the one or more substitutions comprise at least about 1-500 bp. In some embodiments, the one or more substitutions comprise at least about 500-1000 bp. In some embodiments, the one or more substitutions comprise at least about 1000-2000 bp. In some embodiments, the one or more substitutions comprise at least about 1000-2000 bp. In some embodiments, the one or more substitutions comprise at least about 2000-3000 bp. In some embodiments, the one or more substitutions comprise at least about 3000-4000 bp. In some embodiments, the one or more substitutions comprise at least about 4000-5000 bp. In some embodiments, the one or more substitutions comprise at least about 5,000-6,000 bp. In some embodiments, the one or more substitutions comprise at least about 6,000-7,000 bp. In some embodiments, the one or more substitutions comprise at least about 7,000-8,000 bp. In some embodiments, the one or more substitutions comprise at least about 8,000-9,000 bp. In some embodiments, the one or more substitutions comprise at least about 9,000-10,000 bp. In some embodiments, the one or more substitutions comprise at least about 10,000-15,000 bp. In some embodiments, the one or more substitutions comprise at least about 10,000-15,000 bp, at least about 15,000-20,000 bp, at least about 20,000-25,000 bp, at least about 25,000-30,000 bp, at least about 30,000-35,000 bp, at least about 35,000-40,000 bp, at least about 40,000-45,000 bp, at least about 45,000-50,000 bp, at least about 50,000-55,000 bp, at least about 55,000-60,000 bp, at least about 60,000-65,000 bp, at least about 65,000-70,000 bp, at least about 70,000-75,000 bp, at least about 75,000-80,000 bp, at least about 80,000-85,000 bp, at least about 85,000-90,000 bp, at least about 90,000-95,000 bp, at least about 95,000-100,000 bp, at least about 100,000-110,000 bp, at least about 110,000-120,000 bp, at least about 120,000-130,000 bp, at least about 130,000-140,000 bp, at least about 140,000-150,000 bp, at least about 150,000-200,000 bp, or more than at least about 200,000 bp. In one specific embodiment, 9687 bp are substituted. In some embodiments, the substituted nucleotides are interspersed. In some embodiments, the substituted nucleotides are consecutive.

In some embodiments, the one or more substitutions are located within 1-500 bp of the E. coli Nissle Phage 3 genome. In some embodiments, the one or more substitutions are located within 500-1000 bp of the E. coli Nissle Phage 3 genome. In some embodiments, the one or more substitutions are located within at least about 1000-2000 bp of the E. coli Nissle Phage 3 genome. In some embodiments, the one or more substitutions are located within at least about 1000-2000 bp of the E. coli Nissle Phage 3 genome. In some embodiments, the one or more substitutions are located within at least about 2000-3000 bp of the E. coli Nissle Phage 3 genome. In some embodiments, the one or more substitutions are located within at least about 3000-4000 bp of the E. coli Nissle Phage 3 genome. In some embodiments, the one or more substitutions are located within at least about 4000-5000 bp of the E. coli Nissle Phage 3 genome. In some embodiments, the one or more substitutions are located within at least about 5,000-6,000 bp of the E. coli Nissle Phage 3 genome. In some embodiments, the one or more substitutions are located within at least about 6,000-7,000 bp of the E. coli Nissle Phage 3 genome. In some embodiments, the one or more substitutions are located within at least about 7,000-8,000 bp of the E. coli Nissle Phage 3 genome. In some embodiments, the one or more substitutions are located within at least about 8,000-9,000 bp of the E. coli Nissle Phage 3 genome. In some embodiments, the one or more substitutions are located within at least about 9,000-10,000 bp of the E. coli Nissle Phage 3 genome. In some embodiments, the one or more substitutions are located within at least about 10,000-15,000 bp of the E. coli Nissle Phage 3 genome. In some embodiments, the one or more substitutions are located within at least about 10,000-15,000 bp of the E. coli Nissle Phage 3 genome, at least about 15,000-20,000 bp of the E. coli Nissle Phage 3 genome, at least about 20,000-25,000 bp of the E. coli Nissle Phage 3 genome, at least about 25,000-30,000 bp of the E. coli Nissle Phage 3 genome, at least about 30,000-35,000 bp of the E. coli Nissle Phage 3 genome, at least about 35,000-40,000 bp of the E. coli Nissle Phage 3 genome, 40,000-45,000 bp of the E. coli Nissle Phage 3 genome, at least about 45,000-50,000 bp of the E. coli Nissle Phage 3 genome, at least about 50,000-55,000 bp of the E. coli Nissle Phage 3 genome, or at least about 55,000-60,000 bp of the E. coli Nissle Phage 3 genome In one specific embodiment, 9687 bp of the E. coli Nissle Phage 3 genome are substituted. In some embodiments, the substituted nucleotides are interspersed. In some embodiments, the substituted nucleotides are consecutive.

In some embodiments, the substitutions are located within at least about 0.1 to 1%, at least about 1 to 2%, at least about 2 to 3%, at least about 3 to 4%, at least about 4 to 5%, at least about 5 to 6%, at least about 6 to 7%, at least about 7 to 8%, at least about 8 to 9%, at least about 9 to 10%, at least about 10 to 11%, at least about 11 to 12%, at least about 12 to 13%, at least about 13 to 14%, at least about 14 to 15%, at least about 15 to 16, 16 to 17%, at least about 17 to 18%, at least about 18 to 19%, at least about 19 to 20%, at least about 20 to 21%, at least about 21 to 22%, at least about 22 to 23%, at least about 23 to 24%, at least about 24 to 25%, at least about 25 to 26%, at least about 26 to 27%, at least about 27 to 28%, at least about 28 to 29%, at least about or 29 to 30% of the E. coli Nissle Phage 3 genome. In some embodiments, at least about 30-40% of the E. coli Nissle Phage 3 genome is substituted. In some embodiments, the substitutions are located within at least about 40-50% of the E. coli Nissle Phage 3 genome. In some embodiments, the substitutions are located within at least about 50-60% of the E. coli Nissle Phage 3 genome. In some embodiments, the substitutions are located within at least about 60-70% of the E. coli Nissle Phage 3 genome. In some embodiments, the substitutions are located within at least about 70-80% of the E. coli Nissle Phage 3 genome. In some embodiments, the substitutions are located within at least about 80-90% of the E. coli Nissle Phage 3 genome. In some embodiments, the substitutions are located within at least about 90-100% of the E. coli Nissle Phage 3 genome.

In some embodiments, at least about 1, 2, 3, 4, 5, 6, 7, 8, 9, 10, 11, 12, 13, 14, 15, 16, 17, 18, 19, or 20 genes comprise substitutions. In some embodiments, at least about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 genes comprise substitutions. In some embodiments, 13 genes comprise substitutions. In one embodiment, 74 genes comprise substitutions.

In some embodiments, the one or more substitutions are located at the beginning or 5′ end of the E. coli Nissle Phage 3 genome. In some embodiments, the one or more substitutions are located at the end or 3′ end of the E. coli Nissle Phage 3 genome. In some embodiments, the one or more substitutions are located in the middle of the E. coli Nissle Phage 3 genome. In some embodiments, the E. coli Nissle Phage 3 genes are interspersed within the bacterial genome and the substitution are located in one or more of the interspersed positions.

In some embodiments, the region for an optimal substitution, i.e., to achieve a desired effect, can be determined through analysis of homology with other phages is other bacteria. Homologous conserved regions in phages may be suitable for substitution, as these are conserved and may comprise one or more essential genes. In some embodiments, regulatory elements, such as promoters, are substituted. In some embodiments, coding sequences are substituted. In some embodiments, the one or more substituted regions contain one or more genes essential for the lytic cycle.

In some embodiments, the substitutions are located within one or more genes encoding lytic genes. In some embodiments, the substitutions are located within one or more genes encoding one or more proteases or lysins. In some embodiments, the substitutions are located within one or more genes encoding one or more toxins. In some embodiments, the substitutions are located within one or more genes encoding one or more antibiotic resistance related proteins. In some embodiments, the substitutions are located within one or more genes encoding one or phage translation related proteins. In some embodiments, the one or more substitutions are located within one or more genes encoding structural proteins. Such structural genes include genes encoding polypeptides of the head, tail, collar, or coat. In some embodiments, the substitutions are located within one or more genes encoding one or more plate proteins. In some embodiments, the substitutions are located within one or more genes encoding one or more proteins require for assembly of the bacteriophage. In some embodiments, the substitutions are located within one or more genes encoding one or more portal proteins. In some embodiments, the substitutions are located within one or more genes encoding one or more polypeptides involved in recombination. In some embodiments, the substitutions are located within one or more genes encoding one or more integrases. In some embodiments, the substitutions are located within one or more genes encoding one or more invertases. In some embodiments, the substitutions are located within one or more genes encoding one or more transposases. In some embodiments, the substitutions are located with within one or more genes encoding one or more polypeptides involved in replication or translation. In some embodiments, the substitutions are located within one or more genes encoding one or more primases. In some embodiments, the substitutions are located within one or more genes encoding one or more tRNA related proteins. In some embodiments, the substitutions are located within one or more genes encoding one or more polypeptides involved in phage substitution. In some embodiments, the substitutions are located within one or more genes encoding an attachment site. In some embodiments, the substitutions are located within one or more genes encoding one or more polypeptides involved in packaging. In some embodiments, the substitutions are located within one or more genes encoding one or more terminases. In some embodiments, the substitutions are located within one or more genes encoding one or more host genes.

In some embodiments, the substitutions are located within genes encoding one or more polypeptides involved in one or more of cell lysis, phage structure, phage assembly, phage packaging recombination, replication or translation, phage substitution, or are host proteins, and combinations thereof.

In some embodiments, the substitutions are located within genes encoding one or more polypeptides involved in one or more of cell lysis, phage structure, phage assembly, phage packaging recombination, replication or translation, phage substitution, and combinations thereof.

In some embodiments, the substitutions are located within 1 genes encoding polypeptides involved in cell lysis, phage structure, phage assembly, phage packaging recombination, replication or translation, phage substitution, and combinations thereof. In some embodiments, the substitutions are located within 2 genes encoding polypeptides involved in cell lysis, phage structure, phage assembly, phage packaging recombination, replication or translation, phage substitution, and combinations thereof. In some embodiments, the substitutions are located within 3 genes encoding polypeptides involved in cell lysis, phage structure, phage assembly, phage packaging recombination, replication or translation, phage substitution, and combinations thereof. In some embodiments, the substitutions are located within 4 genes encoding polypeptides involved in cell lysis, phage structure, phage assembly, phage packaging recombination, replication or translation, phage substitution, and combinations thereof. In some embodiments, the substitutions are located within 2 genes encoding polypeptides involved in cell lysis, phage structure, phage assembly, phage packaging recombination, replication or translation, phage substitution, and combinations thereof. In some embodiments, the substitutions are located within 5 genes encoding polypeptides involved in cell lysis, phage structure, phage assembly, phage packaging recombination, replication or translation, phage substitution, and combinations thereof. In some embodiments, the substitutions are located within 6 genes encoding polypeptides involved in cell lysis, phage structure, phage assembly, phage packaging recombination, replication or translation, phage substitution, and combinations thereof. In some embodiments, the substitutions are located within 7 genes encoding polypeptides involved in cell lysis, phage structure, phage assembly, phage packaging recombination, replication or translation, phage substitution, and combinations thereof. In some embodiments, the substitutions are located within 8 genes encoding polypeptides involved in cell lysis, phage structure, phage assembly, phage packaging recombination, replication or translation, phage substitution, and combinations thereof. In some embodiments, the substitutions are located within 9 genes encoding polypeptides involved in cell lysis, phage structure, phage assembly, phage packaging recombination, replication or translation, phage substitution, and combinations thereof. In some embodiments, the substitutions are located within 10 genes encoding polypeptides involved in cell lysis, phage structure, phage assembly, phage packaging recombination, replication or translation, phage substitution, and combinations thereof. In some embodiments, the substitutions are located within 11 genes encoding polypeptides involved in cell lysis, phage structure, phage assembly, phage packaging recombination, replication or translation, phage substitution, and combinations thereof. In some embodiments, the substitutions are located within 12 genes encoding polypeptides involved in cell lysis, phage structure, phage assembly, phage packaging recombination, replication or translation, phage substitution, and combinations thereof. In some embodiments, the substitutions are located within 13 genes encoding polypeptides involved in cell lysis, phage structure, phage assembly, phage packaging recombination, replication or translation, phage substitution, and combinations thereof. In some embodiments, the substitutions are located within 14 genes encoding polypeptides involved in cell lysis, phage structure, phage assembly, phage packaging recombination, replication or translation, phage substitution, and combinations thereof. In some embodiments, the substitutions are located within 15 genes encoding polypeptides involved in cell lysis, phage structure, phage assembly, phage packaging recombination, replication or translation, phage substitution, and combinations thereof. In some embodiments, the substitutions are located within at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more genes encoding polypeptides involved in cell lysis, phage structure, phage assembly, phage packaging recombination, replication or translation, phage substitution, and combinations thereof. In some embodiments, the substitutions are located within one or more host proteins within the phage genome.

In any of the embodiments described herein, the substitutions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In some embodiments, one or more substitutions are located in ECOLIN_09965. In some embodiments, one or more substitutions are located in ECOLIN_09970. In some embodiments, one or more substitutions are located in ECOLIN_09975. In some embodiments, one or more substitutions are located in ECOLIN_09980. In some embodiments, one or more substitutions are located in ECOLIN_09985. In some embodiments, one or more substitutions are located in ECOLIN_09990. In some embodiments, one or more substitutions are located in ECOLIN_09995. In some embodiments, one or more substitutions are located in ECOLIN_10000. In some embodiments, one or more substitutions are located in ECOLIN_10005. In some embodiments, one or more substitutions are located in ECOLIN_10010. In some embodiments, one or more substitutions are located in ECOLIN_10015. In some embodiments, one or more substitutions are located in ECOLIN_10020. In some embodiments, one or more substitutions are located in ECOLIN_10025. In some embodiments, one or more substitutions are located in ECOLIN_10030. In some embodiments, one or more substitutions are located in ECOLIN_10035. In some embodiments, one or more substitutions are located in ECOLIN_10040. In some embodiments, one or more substitutions are located in ECOLIN_10045. In some embodiments, one or more substitutions are located in ECOLIN_10050. In some embodiments, one or more substitutions are located in ECOLIN_10055. In some embodiments, one or more substitutions are located in ECOLIN_10065. In some embodiments, one or more substitutions are located in ECOLIN_10070. In some embodiments, one or more substitutions are located in ECOLIN_10075. In some embodiments, one or more substitutions are located in ECOLIN_10080. In some embodiments, one or more substitutions are located in ECOLIN_10085. In some embodiments, one or more substitutions are located in ECOLIN_10090. In some embodiments, one or more substitutions are located in ECOLIN_10095. In some embodiments, one or more substitutions are located in ECOLIN_10100. In some embodiments, one or more substitutions are located in ECOLIN_10105. In some embodiments, one or more substitutions are located in ECOLIN_10110. In some embodiments, one or more substitutions are located in ECOLIN_10115. In some embodiments, one or more substitutions are located in ECOLIN_10120. In some embodiments, one or more substitutions are located in ECOLIN_10125. In some embodiments, one or more substitutions are located in ECOLIN_10130. In some embodiments, one or more substitutions are located in ECOLIN_10135. In some embodiments, one or more substitutions are located in ECOLIN_10140. In some embodiments, one or more substitutions are located in ECOLIN_10145. In some embodiments, one or more substitutions are located in ECOLIN_10150. In some embodiments, one or more substitutions are located in ECOLIN_10160. In some embodiments, one or more substitutions are located in ECOLIN_10165. In some embodiments, one or more substitutions are located in ECOLIN_10170. In some embodiments, one or more substitutions are located in ECOLIN_10175. In some embodiments, one or more substitutions are located in ECOLIN_10180. In some embodiments, one or more substitutions are located in ECOLIN_10185. In some embodiments, one or more substitutions are located in ECOLIN_10190. In some embodiments, one or more substitutions are located in ECOLIN_10195. In some embodiments, one or more substitutions are located in ECOLIN_10200. In some embodiments, one or more substitutions are located in ECOLIN_10205. In some embodiments, one or more substitutions are located in ECOLIN_10210. In some embodiments, one or more substitutions are located in ECOLIN_10220. In some embodiments, one or more substitutions are located in ECOLIN_10225. In some embodiments, one or more substitutions are located in ECOLIN_10230. In some embodiments, one or more substitutions are located in ECOLIN_10235. In some embodiments, one or more substitutions are located in ECOLIN_10240. In some embodiments, one or more substitutions are located in ECOLIN_10245. In some embodiments, one or more substitutions are located in ECOLIN_10250. In some embodiments, one or more substitutions are located in ECOLIN_10255. In some embodiments, one or more substitutions are located in ECOLIN_10260. In some embodiments, one or more substitutions are located in ECOLIN_10265. In some embodiments, one or more substitutions are located in ECOLIN_10270. In some embodiments, one or more substitutions are located in ECOLIN_10275. In some embodiments, one or more substitutions are located in ECOLIN_10280. In some embodiments, one or more substitutions are located in ECOLIN_10290. In some embodiments, one or more substitutions are located in ECOLIN_10295. In some embodiments, one or more substitutions are located in ECOLIN_10300. In some embodiments, one or more substitutions are located in ECOLIN_10305. In some embodiments, one or more substitutions are located in ECOLIN_10310. In some embodiments, one or more substitutions are located in ECOLIN_10315. In some embodiments, one or more substitutions are located in ECOLIN_10320. In some embodiments, one or more substitutions are located in ECOLIN_10325. In some embodiments, one or more substitutions are located in ECOLIN_10330. In some embodiments, one or more substitutions are located in ECOLIN_10335. In some embodiments, one or more substitutions are located in ECOLIN_10340. In some embodiments, one or more substitutions are located in ECOLIN_10345.

In some embodiments, the mutations are located in or encompass one or more polypeptides selected from lipid A biosynthesis (KDO)2-(lauroyl)-lipid IVA acyltransferase, peptidase, zinc ABC transporter substrate-binding protein, zinc ABC transporter ATPase, high-affinity zinc transporter membrane component, ATP-dependent DNA helicase RuvB, ATP-dependent DNA helicase RuvA, Holliday junction resolvase, dihydroneopterin triphosphate pyrophosphatase, aspartyl-tRNA synthetase, hydrolase, DNA polymerase V, MsgA, phage tail protein, tail protein, host specificity protein, peptidase P60, tail protein, tail protein, tail fiber protein, Minor tail protein U, DNA breaking-rejoining protein, peptidase S14, capsid protein, DNA packaging protein, terminase, lysozyme, holin, DNA adenine methylase, serine protease, antitermination protein, antirepressor, crossover junction endodeoxyribonuclease, adenine methyltransferase, DNA methyltransferase ECOLIN_10240, GntR family transcriptional regulator ECOLIN_10245, cI repressor, Domain of unknown function (DUF4222); DNA recombinase, Multiple Antibiotic Resistance Regulator (MarR), unknown ead like protein in P22, Protein of unknown function (DUF550); 3′-5′ exonuclease, excisionase, integrase, and tRNA methyltransferase. In one embodiment, one or more of a Minor tail protein U, a tail protein, a DNA breaking-rejoining protein, a peptidase S14, a capsid protein, a DNA packaging protein, and a terminase contain one or more substitutions. In one specific embodiment, a Minor tail protein U, a tail protein, a DNA breaking-rejoining protein, a peptidase S14, a capsid protein, a DNA packaging protein, and a terminase contain one or more substitutions.

In one embodiment, the substitution is a complete or partial substitution of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the substitution is a complete or partial substitution of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the substitution is a complete substitution of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial substitution of ECOLIN_10175. In one embodiment, the sequence of SEQ ID NO: 130 is substituted from the Phage 3 genome. In one embodiment, a sequence comprising SEQ ID NO: 130 is substituted from the Phage 3 genome.

### Regulation of Effector Molecules and Payloads Expression

In some embodiments, the bacterial cell which comprises a mutated endogenous phage further comprises a stably maintained plasmid or chromosome carrying the gene(s) encoding payload (s), such that the payload(s) can be expressed in the host cell, and the host cell is capable of survival and/or growth in vitro, e.g., in medium, and/or in vivo, e.g., in the gutor the tumor microenvironment. In some embodiments, bacterial cell comprises two or more distinct payloads or operons, e.g., two or more payload genes. In some embodiments, bacterial cell comprises three or more distinct transporters or operons, e.g., three or more payload genes. In some embodiments, bacterial cell comprises at least about 4, 5, 6, 7, 8, 9, 10, or more distinct payloads or operons, e.g., at least about 4, 5, 6, 7, 8, 9, 10, or more payload genes.

In one embodiment, the genetically engineered bacteria of the invention comprise a gene encoding a phenylalanine-metabolizing enzyme (PME). In some embodiments, the genetically engineered bacteria comprise a gene encoding a phenylalanine-metabolizing enzyme (PME) and are capable of reducing hyperphenylalaninemia.

Examples of phenylalanine metabolizing enzymes include, but are not limited to, phenylalanine hydroxylase (PAH), phenylalanine ammonia lyase (PAL), aminotransferases, L-amino acid deaminase (LAAD), and phenylalanine dehydrogenases. Reactions with phenylalanine hydroxylases, phenylalanine dehydrogenases or aminotransferases require cofactors, while LAAD and PAL do not require any extra cofactor. Without wishing to be bound by theory, the lack of need for a cofactor means that phenylalanine degradation by the enzyme encoded by the genetically engineered bacteria is dependent on the availability of the substrate and is not limited by the availability of the cofactor.

In some embodiments, the engineered bacteria comprise gene sequence encoding one or more phenylalanine hydroxylase (PAH) polypeptides. In some embodiments, the engineered bacteria comprise gene sequence encoding one or more phenylalanine ammonia lyase (PAL) polypeptides. Phenylalanine ammonia lyase (PAL; EC 4.3.1.24) is an enzyme that catalyzes a reaction converting L-phenylalanine to ammonia and trans-cinnamic acid. Phenylalanine ammonia lyase is specific for L-Phe, and to a lesser extent, L-Tyrosine. The reaction catalyzed by PAL is the spontaneous, non-oxidative deamination of L-phenylalanine to yield trans-cinnamic acid and ammonia. Unlike the mammalian enzyme (PAH), PAL is a monomer and requires no cofactors (MacDonald et al., Biochem Cell Biol 2007; 85:273-82. A modern view of phenylalanine ammonia lyase). In micro-organisms, it has a catabolic role, allowing them to utilize L-phenylalanine (L-Phe) as a sole source of carbon and nitrogen. In one embodiment, the genetically engineered bacteria of the invention comprise a PAL gene. PAL is capable of converting phenylalanine to non-toxic levels of transcinnamic acid and ammonia. Trans-cinnamic acid (TCA) can further be converted to TCA metabolites benzoic and hippuric acids (Sarkissian et al., J Mass Spectrom. 2007 June; 42(6):811-7; Quantitation of phenylalanine and its trans-cinnamic, benzoic and hippuric acid metabolites in biological fluids in a single GC-MS analysis). PAL enzyme activity does not require THB cofactor activity.

In some embodiments, PAL is encoded by a PAL gene derived from a bacterial species, including but not limited to, Achromobacter xylosoxidans, Pseudomonas aeruginosa, Photorhabdus luminescens, Anabaena variabilis, and Agrobacterium tumefaciens. In some embodiments, the bacterial species is Photorhabdus luminescens. In some embodiments, the bacterial species is Anabaena variabilis. In some embodiments, PAL is encoded by a PAL gene derived from a eukaryotic species, e.g., a yeast species, a plant species. Multiple distinct PAL proteins are known in the art. The genetically engineered bacteria convert more phenylalanine when the PAL gene is expressed than unmodified bacteria of the same bacterial subtype under the same conditions. Thus, the genetically engineered bacteria comprising PAL may be used to metabolize phenylalanine in the body into non-toxic molecules in order to treat conditions associated with hyperphenylalaninemia, including PKU. In some embodiments, the genetically engineered bacteria express Anabaena variabilis PAL (“PAL1”). In some embodiments, the genetically engineered bacteria express Photorhabdus luminescens PAL (“PAL3”). Non-limiting examples of PAL sequences of interest are shown in Table 2.

In some embodiments, the engineered bacteria comprise gene sequence encoding one or more LAAD polypeptides. In some embodiments, the engineered bacteria comprise gene sequence encoding one or more PAL polypeptides and one or more LAAD polypeptides. LAAD catalyzes the stereospecific oxidative, i.e., oxygen consuming, deamination of L-amino acids to α-keto acids along with the production of ammonia and hydrogen peroxide via an imino acid intermediate. LAADs are found in snake venoms, and in many bacteria (Bifulco et al. 2013), specifically in the cytomembranes of the Proteus, Providencia, and Morganella bacteria. LAADs (EC 1.4.3.2) are flavoenzymes with a dimeric structure. Each subunit contains a non-covalently-bound flavin adenine dinucleotide (FAD) cofactor) and do not require any external cofactors. Proteus mirabilis contains two types of LAADs (Duerre and Chakrabarty 1975). One has broad substrate specificity and catalyzes the oxidation of aliphatic and aromatic L-amino acids to keto acids, typically L-phenylalanine (GenBank: U35383.1) (Baek et al., Journal of Basic Microbiology 2011, 51, 129-135; “Expression and characterization of a second L-amino acid deaminase isolated from Proteus mirabilis in Escherichia coli”). The other type acts mainly on basic L-amino acids (GenBank: EU669819.1). LAADs from bacterial, fungal, and plant sources appear to be involved in the utilization of L-amino acids (i.e., ammonia produced by the enzymatic activity) as a nitrogen source. Most eukaryotic and prokaryotic L-amino acid deaminases are extracellularly secreted, with the exception of from Proteus species LAADs, which are membrane-bound. In Proteus mirabilis, LAADs have been reported to be located in the plasma membrane, facing outward into the periplasmic space, in which the enzymatic activity resides (Pelmont J et al., (1972) “L-amino acid oxidases of Proteus mirabilis: general properties” Biochimie 54: 1359-1374).

In one embodiment, the genetically engineered bacteria of the invention comprise a LAAD gene. LAAD is capable of converting phenylalanine to non-toxic levels of phenylpyruvate, which can also further be degraded, e.g., by liver enzymes, to phenyllactate. Phenylpyruvate cannot cross the blood brain barrier, which allows LAAD to reduce the levels of phenylalanine in the brain without allowing the accumulation of another potentially toxic metabolite. In some embodiments, LAAD is encoded by a LAAD gene derived from a bacterial species, including but not limited to, Proteus, Providencia, and Morganella bacteria. In some embodiments, the bacterial species is Proteus mirabilis. In some embodiments, the bacterial species is Proteus vulgaris. In some embodiments, the genetically engineered bacteria express Proteus mirabilis LAAD enzyme GenBank: U35383.1. Non-limiting examples of LAAD sequences of interest are shown in Table 2. In some embodiments, the LAAD enzyme is derived from snake venom. According to the invention, genetically engineered bacteria convert more phenylalanine when the LAAD gene is expressed than unmodified bacteria of the same bacterial subtype under the same conditions. Thus, the genetically engineered bacteria comprising LAAD may be used to metabolize phenylalanine in the body into non-toxic molecules in order to treat conditions associated with hyperphenylalaninemia, including PKU.

In some embodiments, the genetically engineered bacteria encode a wild type enzyme as it occurs in nature. In some embodiments, the genetically engineered bacteria encode an enzyme which comprises mutations relative to the wild type sequence. In some embodiments, the mutations increase stability of the enzyme. In some embodiments, the mutations increase the catalytic activity of the enzyme. In some embodiments, the genetically engineered bacteria comprise a gene encoding one or more of the proteins listed in Table 2. In some embodiments, the genetically engineered bacteria comprise gene sequence(s) encoding one or more of the polypeptides comprising sequence of any of SEQ ID Nos: 1-8. In some embodiments, the genetically engineered bacteria comprise gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID Nos: 1-8. In some embodiments, the genetically engineered bacteria encode one or more enzymes from Table 2, which comprise a mutation. In some embodiments, the genetically engineered bacteria comprise a gene encoding wild type PAH. In some embodiments, the genetically engineered bacteria encode a mutated PAH with increased stability and/or activity. In some embodiments, the genetically engineered bacteria comprise a gene encoding wild type PAL. In some embodiments, the genetically engineered bacteria encode a mutated PAL with increased stability and/or activity. In some embodiments, the genetically engineered bacteria comprise a gene encoding wild type LAAD. In some embodiments, the genetically engineered bacteria encode a mutated LAAD with increased stability and/or activity. Methods for screening for enzymes with desirable properties are known in the art and described herein.

TABLE 2
Sequences of Phenylalanine Metabolizing Enzymes

| | | SEQ ID |
|---|---|---|
| Description | Sequence | NO |
| Phenylalanine | MKTLSQAQSKTSSQQFSFTGNSSANVIIGNQKLTINDVA | SEQ ID |
| ammonia- | RVARNGTLVSLTNNTDILQGIQASCDYINNAVESGEPIY | NO: 1 |
| lyase | GVTSGFGGMANVAISREQASELQTNLVWFLKTGAGNKL | |
| (Anabaena | PLADVRAAMLLRANSHMRGASGIRLELIKRMEIFLNAG | |
| variabilis) | VTPYVYEFGSIGASGDLVPLSYITGSLIGLDPSFKVDFNG | |
| Acc. No.: | KEMDAPTALRQLNLSPLTLLPKEGLAMMNGTSVMTGIA | |
| Q3M5Z3.1 | ANCVYDTQILTAIAMGVHALDIQALNGTNQSFHPFIHNS | |
| | KPHPGQLWAADQMISLLANSQLVRDELDGKHDYRDHE | |
| | LIQDRYSLRCLPQYLGPIVDGISQIAKQIEIEINSVTDNPLI | |
| | DVDNQASYHGGNFLGQYVGMGMDHLRYYIGLLAKHL | |
| | DVQIALLASPEFSNGLPPSLLGNRERKVNMGLKGLQICG | |
| | NSIMPLLTFYGNSIADRFPTHAEQFNQNINSQGYTSATLA | |
| | RRSVDIFQNYVAIALMFGVQAVDLRTYKKTGHYDARA | |
| | CLSPATERLYSAVRHVVGQKPTSDRPYIWNDNEQGLDE | |
| | HIARISADIAAGGVIVQAVQDILPCLH | |
| histidine | MKTLSQAQSKTSSQQFSFTGNSSANVIIGNQKLTINDVA | SEQ ID |
| ammonia- | RVARNGTLVSLTNNTDILQGIQASCDYINNAVESGEPIY | NO: 2 |
| lyase | GVTSGFGGMANVAISREQASELQTNLVWFLKTGAGNKL | |
| [Anabaena | PLADVRAAMLLRANSHMRGASGIRLELIKRMEIFLNAG | |
| variabilis | VTPYVYEFGSIGASGDLVPLSYITGSLIGLDPSFKVDFNG | |
| ATCC 29413] | KEMDAPTALRQLNLSPLTLLPKEGLAMMNGTSVMTGIA | |
| (Acc. NO: | ANCVYDTQILTAIAMGVHALDIQALNGTNQSFHPFIHNS | |
| ABA23593.1) | KPHPGQLWAADQMISLLANSQLVRDELDGKHDYRDHE | |
| | LIQDRYSLRCLPQYLGPIVDGISQIAKQIEIEINSVTDNPLI | |
| | DVDNQASYHGGNFLGQYVGMGMDHLRYYIGLLAKHL | |
| | DVQIALLASPEFSNGLPPSLLGNRERKVNMGLKGLQICG | |
| | NSIMPLLTFYGNSIADRFPTHAEQFNQNINSQGYTSATLA | |
| | RRSVDIFQNYVAIALMFGVQAVDLRTYKKTGHYDARA | |
| | CLSPATERLYSAVRHVVGQKPTSDRPYIWNDNEQGLDE | |
| | HIARISADIAAGGVIVQAVQDILPCLH | |
| histidine | MKAKDVQPTIIINKNGLISLEDIYDIAIKQKKVEISTEITEL | SEQ ID |
| ammonia- | LTHGREKLEEKLNSGEVIYGINTGFGGNANLVVPFEKIA | NO: 3 |
| lyase | EHQQNLLTFLSAGTGDYMSKPCIKASQFTMLLSVCKGW | |
| [Photorhabdus | SATRPIVAQAIVDHINHDIVPLVPRYGSVGASGDLIPLSYI | |
| luminescens] | ARALCGIGKVYYMGAEIDAAEAIKRAGLTPLSLKAKEG | |
| (WP_ | LALINGTRVMSGISAITVIKLEKLFKASISAIALAVEALLA | |
| 011146484) | SHEHYDARIQQVKNHPGQNAVASALRNLLAGSTQVNLL | |
| | SGVKEQANKACRHQEITQLNDTLQEVYSIRCAPQVLGIV | |
| | PESLATARKILEREVISANDNPLIDPENGDVLHGGNFMG | |
| | QYVARTMDALKLDIALIANHLHAIVALMMDNRFSRGLP | |
| | NSLSPTPGMYQGFKGVQLSQTALVAAIRHDCAASGIHTL | |
| | ATEQYNQDIVSLGLHAAQDVLEMEQKLRNIVSMTILVV | |
| | CQAIHLRGNISEIAPETAKFYHAVREISSPLITDRALDEDII | |
| | RIADAIINDQLPLPEIMLEE | |
| Histidine | MKQLTIYPGKLTLDELRQVYLQPVKITLDSQIFPAIERSV | SEQ ID |
| ammonia | ECVNAILAENRTAYGINTGFGLLASTRIEEDNLEKLQRSL | NO: 4 |
| lyase | VVSHAAGVGKALDDNMTRLIMVLKINSLSRGYSGIRLA | |
| (Photorhabdus | VIQALIALVNAEIYPHIPCKGSVGASGDLAPLAHMSLLLL | |
| luminescens) | GEGQARYQGEWLPAKEALAKANLQPITLAAKEGLALLN | |
| Acc. NO: | GTQVSTAFALRGLFEAEDLLAAAIVCGSLSVEAALGSRK | |
| CAE15566 | PFDARVHVVRGQQGQIDVAALYRHVLEESSELSDSHINC | |
| | PKVQDPYSLRCQPQVMGACLTQLRHAADVILTEANAVS | |
| | DNPLVFAEQGEVISGGNFHAEPVAMASDNLALVLAEIG | |
| | ALSERRIALLMDSHMSQLPPFLVENGGVNSGFMIAQVTA | |
| | AALASENKALAHPASVDSLPTSANQEDHVSMAPAAGRR | |
| | LWEMAENTRGILAIEWLSACQGIDFRNGLKSSPILEEAR | |
| | VILRAKVDYYDQDRFFAPDIDAAVKLLAEQHLSSLLPSG | |
| | QILQRKNNR | |
| amino acid | MAISRRKFILGGTVVAVAAGAGVLTPMLTREGRFVPGT | SEQ ID |
| deaminase | PRHGFVEGTGGPLPKQDDVVVIGAGILGIMTAINLAERG | NO: 5 |
| (Proteus | LSVTIVEKGNIAGEQSSRFYGQAISYKMPDETFLLHHLG | |
| mirabilis) | KHRWREMNAKVGIDTTYRTQGRVEVPLDEEDLENVRK | |
| Acc. No: | WIDAKSKDVGSDIPFRTKMIEGAELKQRLRGATTDWKI | |
| ACD36582 | AGFEEDSGSFDPEVATFVMAEYAKKMGIKIFTNCAARG | |
| | LETQAGVISDVVTEKGPIKTSRVVVAGGVGSRLFMQNL | |
| | NVDVPTLPAYQSQQLISAAPNAPGGNVALPGGIFFRDQA | |
| | DGTYATSPRVIVAPVVKESFTYGYKYLPLLALPDFPVHIS | |
| | LNEQLINSFMQSTHWDLNEESPFEKYRDMTALPDLPELN | |
| | ASLEKLKKEFPAFKESTLIDQWSGAMAIAPDENPIISDVK | |
| | EYPGLVINTATGWGMTESPVSAEITADLLLGKKPVLDAK | |
| | PFSLYRF | |
| amino acid | MNISRRKLLLGVGAAGVLAGGAALVPMVRRDGKFVEA | SEQ ID |
| deaminase | KSRASFVEGTQGALPKEADVVIIGAGIQGIMTAINLAERG | NO: 6 |
| [Proteus | MSVTILEKGQIAGEQSGRAYSQIISYQTSPEIFPLHHYGKI | |
| mirabilis | LWRGMNEKIGADTSYRTQGRVEALADEKALDKAQAWI | |
| HI4320]) | KTAKEAAGFDTPLNTRIIKGEELSNRLVGAQTPWTVAAF | |
| Acc. No.: | EEDSGSVDPETGTPALARYAKQIGVKIYTNCAVRGIETA | |
| AAA86752.1 | GGKISDVVSEKGAIKTSQVVLAGGIWSRLFMGNMGIDIP | |
| | TLNVYLSQQRVSGVPGAPRGNVHLPNGIHFREQADGTY | |
| | AVAPRIFTSSIVKDSFLLGPKFMHLLGGGELPLEFSIGEDL | |
| | FNSFKMPTSWNLDEKTPFEQFRVATATQNTQHLDAVFQ | |
| | RMKTEFPVFEKSEVVERWGAVVSPTFDELPIISEVKEYP | |
| | GLVINTATVWGMTEGPAAGEVTADIVMGKKPVIDPTPF | |
| | SLDRFKK | |
| LAAD from | MAISRRKFIIGGTVVAVAAGAGILTPMLTREGRFVPGTP | SEQ ID |
| Proteus | RHGFVEGTEGALPKQADVVVVGAGILGIMTAINLVERG | NO: 7 |
| vulgaris; | LSVVIVEKGNIAGEQSSRFYGQAISYKMPDETFLLHHLG | |
| (Acc. NO: | KHRWREMNAKVGIDTTYRTQGRVEVPLDEEDLVNVRK | |
| BAA90864) | WIDERSKNVGSDIPFKTRIIEGAELNQRLRGATTDWKIAG | |
| | FEEDSGSFDPEVATFVMAEYAKKMGVRIYTQCAARGLE | |
| | TQAGVISDVVTEKGAIKTSQVVVAGGVWSRLFMQNLN | |
| | VDVPTLPAYQSQQLISGSPTAPGGNVALPGGIFFREQAD | |
| | GTYATSPRVIVAPVVKESFTYGYKYLPLLALPDFPVHISL | |
| | NEQLINSFMQSTHWNLDEVSPFEQFRNMTALPDLPELNA | |
| | SLEKLKAEFPAFKESKLIDQWSGAMAIAPDENPIISEVKE | |
| | YPGLVINTATGWGMTESPVSAELTADLLLGKKPVLDPK | |
| | PFSLYRF | |
| Phenylalanine | MSTAVLENPGLGRKLSDFGQETSYIEDNCNQNGAISLIFS | SEQ ID |
| hydroxylase | LKEEVGALAKVLRLFEENDVNLTHIESRPSRLKKDEYEF | NO: 8 |
| [Homo | FTHLDKRSLPALTNIIKILRHDIGATVHELSRDKKKDTVP | |
| sapiens] | WFPRTIQELDRFANQILSYGAELDADHPGFKDPVYRARR | |
| (Acc. No. | KQFADIAYNYRHGQPIPRVEYMEEGKKTWGTVFKTLKS | |
| AAH26251 | LYKTHACYEYNHIFPLLEKYCGFHEDNIPQLEDVSQFLQ | |
| | TCTGFRLRPVAGLLSSRDFLGGLAFRVFHCTQYIRHGSK | |
| | PMYTPEPDICHELLGHVPLFSDRSFAQFSQEIGLASLGAP | |
| | DEYIEKLATIYWFTVEFGLCKQGDSIKAYGAGLLSSFGE | |
| | LQYCLSEKPKLLPLELEKTAIQNYTVTEFQPLYYVAESF | |
| | NDAKEKVRNFAATIPRPFSVRYDPYTQRIEVLDNTQQLK | |
| | ILADSINSEIGILCSALQKIK | |

The PME, e.g., PAL, LAAD, or PAH, gene(s) may be present on a plasmid or chromosome in the genetically engineered bacteria. In some embodiments, the PME gene sequence(s) are expressed under the control of one or more constitutive promoter(s). In some embodiments, the PME gene is expressed under the control of a promoter that is directly or indirectly induced by exogenous environmental conditions, as described herein. In some embodiments, the PME gene is expressed under the control of a promoter that is directly or indirectly induced by exogenous environmental conditions, such as in the presence of molecules or metabolites specific to the gut of a mammal. In one embodiment, the PME gene is expressed under the control of a promoter that is directly or indirectly induced by low-oxygen, microaerobic, or anaerobic conditions, wherein expression of the PME gene, e.g., the PAL gene, is activated under low-oxygen or anaerobic environments, such as the environment of the mammalian gut.

In some embodiments, the genetically engineered bacteria comprise gene sequence encoding one or more PAL polypeptide sequence(s). In some embodiments, the engineered bacteria comprise gene sequence encoding one or more PAL polypeptide sequence(s) in which the gene sequence(s) is directly or indirectly induced by low-oxygen or anaerobic conditions, such as the mammalian gut. In some embodiments, the engineered bacteria comprise gene sequence encoding one or more LAAD polypeptides. In some embodiments, the engineered bacteria comprise gene sequence encoding one or more LAAD polypeptides, in which the gene sequence(s) is directly or indirectly induced by oxygenated, low oxygen, or microaerobic conditions, such as conditions found in the proximal intestine, including but not limited to the stomach, duodenum, and ileum. In other embodiments, the engineered bacteria comprise gene sequence(s) encoding one or more PME polypeptide sequences(s) in which the gene sequene(s) is directly or indirectly induced by an environmental factor that is naturally present in a mammalian gut. In other embodiments, the genetically engineered bacteria encode one or more PME gene sequences(s) which are directly or indirectly induced by an environmental factor that is not naturally present in a mammalian gut, e.g., arabinose or IPTG. In other embodiments, the genetically engineered bacteria encode one or more PME gene sequences(s) which are directly or indirectly induced by an environmental factor that is naturally present in a mammalian gut under inflammatory conditions. In some embodiments, the engineered bacteria comprise gene sequence(s) encoding one or more PAL polypeptides and gene sequence(s) encoding one or more LAAD polypeptides in which the gene sequences are under the control of the same promoter or a different copy of the same promoter, which is directly or indirectly induced by exogenous environmental conditions, such as any of the environmental conditions discussed herein and such as any of the promoters discussed herein. In some embodiments, the engineered bacteria comprise gene sequence(s) encoding one or more PAL polypeptides and gene sequence(s) encoding one or more LAAD polypeptides in which the gene sequences are under the control of a different promoter, which is directly or indirectly induced by exogenous environmental conditions, such as any of the environmental conditions discussed herein and such as any of the promoters discussed herein. In some embodiments, the engineered bacteria comprise gene sequence(s) encoding one or more PAL polypeptides and gene sequence(s) encoding one or more LAAD polypeptides in which the gene sequences are under the control of a constitutive promoter. In some embodiments, the engineered bacteria comprise gene sequence(s) encoding one or more PAL polypeptides and gene sequence(s) encoding one or more LAAD polypeptides in which the PAL gene sequences are under the control of a constitutive promoter and the LAAD gene sequence(s) are under the control of an inducible promoter. In some embodiments, the engineered bacteria comprise gene sequence(s) encoding one or more PAL polypeptides and gene sequence(s) encoding one or more LAAD polypeptides in which the LAAD gene sequences are under the control of a constitutive promoter and the PAL gene sequence(s) are under the control of an inducible promoter. In any of these embodiments, the bacteria may further comprise gene sequence encoding one or more Phe transporter polypeptides, which gene sequence(s) may be under the control of a constitutive or inducible promoter and may be the same or different promoter from the promoter controlling the Pal and/or LAAD gene sequence(s).

In other embodiments, the engineered bacteria encode one or more PME gene sequence(s) which are directly or indirectly induced prior to in vivo administration during bacterial cell culture; i.e., one or more PME gene sequence(s) are expressed under the control of an inducible promoter that is responsive to specific molecules or metabolites, temperature, oxygen levels or other parameters provided in the culture of the bacterium as it is grown in a flask, fermenter, or other culture vessel. In some embodiments, the engineered bacteria encode one or more PME gene sequence(s) which are directly or indirectly induced prior to in vivo administration during bacterial cell culture; wherein the one or more PME gene sequence(s) are expressed under low oxygen or anaerobic conditions. In some embodiments, the engineered bacteria encode one or more PME gene sequence(s) which are directly or indirectly induced prior to in vivo administration during bacterial cell culture; wherein the one or more PME gene sequence(s) are expressed under aerobic conditions. In some embodiments, the engineered bacteria encode one or more PME gene sequence(s) which are directly or indirectly induced prior to in vivo administration during bacterial cell culture; wherein the one or more PME gene sequence(s) are expressed under microaerobic conditions. In some embodiments, the engineered bacteria encode one or more PME gene sequence(s) which are directly or indirectly induced prior to in vivo administration during bacterial cell culture; wherein the one or more PME gene sequence(s) are expressed in the presence of arabinose. In some embodiments, the engineered bacteria encode one or more PME gene sequence(s) which are directly or indirectly induced prior to in vivo administration during bacterial cell culture; wherein the one or more PME gene sequence(s) are expressed in the presence of IPTG.

Payload (and/or polypeptides of interest and/or proteins of interest and/or therapeutic polypeptides and/or therapeutic proteins and/or therapeutic peptides and/or effector and/or effector molecules) include any of the metabolites described herein and/or any of the enzyme(s) or polypeptide(s) which function as enzymes for the production or catabolism of such effector molecules. Effector molecules and payloads include but are not limited to anti-cancer molecules, immune modulators, gut barrier enhancer molecules, anti-inflammatory molecules, satiety molecules or neuromodulatory effectors. Non-limiting examples of payloads are described in pending, co-owned International Patent Applications PCT/US2016/34200, filed May 25, 2016, PCT/US2017/013072, filed Jan. 11, 2017, PCT/US2017/016603, filed Feb. 3, 2017, PCT/US2017/016609, filed Feb. 4, 2016, PCT/US2017/017563, filed Feb. 10, 2017, PCT/US2017/017552, filed Feb. 10, 2017, PCT/US2016/044922, filed Jul. 29, 2016, PCT/US2016/049781, filed Aug. 31, 2016, PCT/US2016/37098, filed Jun. 10, 2016, PCT/US2016/069052, filed Dec. 28, 2016, PCT/US2016/32562, filed May 13, 2016, PCT/US2016/062369, filed Nov. 16, 2016, and PCT/US2017/013072, the contents of which are herein incorporated by reference in their entireties.

As used herein, the term “gene of interest” or “gene sequence of interest” includes any or a plurality of any of the gene(s) an/or gene sequence(s) and or gene cassette(s) encoding one or more effector molecules and payloads include but are not limited to anti-cancer molecules, immune modulators, gut barrier enhancer molecules, anti-inflammatory molecules, satiety molecules or effectors, neuromodulatory molecules described herein, e.g., kynureninase, tryptophan production enzymes, tryptophan degradation enzymes, one or more kynurenine production enzymes, serotonin or melatonin production or degradation enzymes, indole metabolite production or degradation enzymes (described herein) KP metabolite production or degradation enzymes. Non-limiting examples of additional genes of interest are described in Non-limiting examples of payloads are described in pending, co-owned International Patent Applications PCT/US2016/34200, filed May 25, 2016, PCT/US2017/013072, filed Jan. 11, 2017, PCT/US2017/016603, filed Feb. 3, 2017, PCT/US2017/016609, filed Feb. 4, 2016, PCT/US2017/017563, filed Feb. 10, 2017, PCT/US2017/017552, filed Feb. 10, 2017, PCT/US2016/044922, filed Jul. 29, 2016, PCT/US2016/049781, filed Aug. 31, 2016, PCT/US2016/37098, filed Jun. 10, 2016, PCT/US2016/069052, filed Dec. 28, 2016, PCT/US2016/32562, filed May 13, 2016, PCT/US2016/062369, filed Nov. 16, 2016, and PCT/US2017/013072, the contents of which are herein incorporated by reference in their entireties.

In some embodiments, the genetically engineered bacteria comprise multiple copies of the same payload gene(s). In some embodiments, the gene encoding the payload is present on a plasmid and operably linked to a directly or indirectly inducible promoter. In some embodiments, the gene encoding the payload is present on a plasmid and operably linked to a constitutive promoter. In some embodiments, the gene encoding the payload is present on a plasmid and operably linked to a promoter that is induced under low-oxygen or anaerobic conditions. In some embodiments, the gene encoding the payload is present on plasmid and operably linked to a promoter that is induced by exposure to tetracycline or arabinose, or another chemical or nutritional inducer described herein.

In some embodiments, the gene encoding the payload is present on a chromosome and operably linked to a directly or indirectly inducible promoter. In some embodiments, the gene encoding the payload is present on a chromosome and operably linked to a constitutive promoter. In some embodiments, the gene encoding the payload is present in the chromosome and operably linked to a promoter that is induced under low-oxygen or anaerobic conditions. In some embodiments, the gene encoding the payload is present on chromosome and operably linked to a promoter that is induced by exposure to tetracycline or arabinose, or another chemical or nutritional inducer described herein.

In some embodiments, the genetically engineered bacteria comprise two or more payloads, all of which are present on the chromosome. In some embodiments, the genetically engineered bacteria comprise two or more payloads, all of which are present on one or more same or different plasmids. In some embodiments, the genetically engineered bacteria comprise two or more payloads, some of which are present on the chromosome and some of which are present on one or more same or different plasmids.

In any of the nucleic acid embodiments, described above, the one or more payload(s) for producing a polypeptide of interest combinations are operably linked to one or more directly or indirectly inducible promoter(s). In some embodiments, the one or more payload(s) are operably linked to a directly or indirectly inducible promoter that is induced under exogeneous environmental conditions, e.g., conditions found in the gut, the tumor microenvironment, or other tissue specific conditions. In some embodiments, the one or more payload(s) are operably linked to a directly or indirectly inducible promoter that is induced by metabolites found in the gut, the tumor microenvironment, or other specific conditions. In some embodiments, the one or more payload(s) are operably linked to a directly or indirectly inducible promoter that is induced under low-oxygen or anaerobic conditions. In some embodiments, the one or more payload(s) are operably linked to a directly or indirectly inducible promoter that is induced under inflammatory conditions (e.g., RNS, ROS), as described herein. In some embodiments, the one or more payload(s) are operably linked to a directly or indirectly inducible promoter that is induced under immunosuppressive conditions, e.g., as found in the tumor, or other specific tissues, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, or others described herein. In some embodiments, the two or more payloads are all linked to a constitutive promoter.

In a non-limiting example, the genetically engineered bacteria may comprise two payloads, one of which is linked to a constitutive promoter, and one of which is linked to a directly or indirectly inducible promoter. In a non-limiting example, the genetically engineered bacteria may comprise three payloads, one of which is linked to a constitutive promoter, and one of which is linked to a directly or indirectly inducible promoter and one of which is linked to a second, different directly or indirectly inducible promoter.

In some embodiments, the promoter is induced under in vivo conditions, e.g., the gut, as described herein. In some embodiments, the promoters are induced under in vitro conditions, e.g., various cell culture and/or cell manufacturing conditions, as described herein. In some embodiments, the promoter is induced under in vivo conditions, e.g., the gut, as described herein, and under in vitro conditions, e.g., various cell culture and/or cell production and/or manufacturing conditions, as described herein.

In some embodiments, the promoter that is operably linked to the gene encoding the payload is directly induced by exogenous environmental conditions (e.g., in vivo and/or in vitro and/or production/manufacturing conditions). In some embodiments, the promoter that is operably linked to the gene encoding the payload is indirectly induced by exogenous environmental conditions (e.g., in vivo and/or in vitro and/or production/manufacturing conditions).

In some embodiments, the promoter is directly or indirectly induced by exogenous environmental conditions specific to the gut of a mammal. In some embodiments, the promoter is directly or indirectly induced by exogenous environmental conditions specific to the hypoxic environment of a tumor and/or the small intestine of a mammal. In some embodiments, the promoter is directly or indirectly induced by low-oxygen or anaerobic conditions such as the hypoxic environment of a tumor and/or the environment of the mammalian gut. In some embodiments, the promoter is directly or indirectly induced by molecules or metabolites that are specific to the tumor, a particular tissue, or the gut of a mammal. In some embodiments, the promoter is directly or indirectly induced by a molecule that is co-administered with the bacterial cell.

### FNR Dependent Regulation

The genetically engineered bacteria of the invention comprise a gene or gene cassette for producing a polypeptide of interest, wherein the gene or gene cassette is operably linked to a directly or indirectly inducible promoter that is controlled by exogenous environmental condition(s). In some embodiments, the inducible promoter is an oxygen level-dependent promoter and a polypeptide of interest is expressed in low-oxygen, microaerobic, or anaerobic conditions. For example, in low oxygen conditions, the oxygen level-dependent promoter is activated by a corresponding oxygen level-sensing transcription factor, thereby driving production of the polypeptide of interest.

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the genetically engineered bacteria comprise a gene or gene cassette for producing a payload under the control of an oxygen level-dependent promoter. In a more specific aspect, the genetically engineered bacteria comprise a gene or gene cassette for producing a payload under the control of an oxygen level-dependent promoter that is activated under low-oxygen or anaerobic environments, such as the hypoxic environment of a tumor and/or the environment of the mammalian gut, and/or other specific tissues.

In certain embodiments, the bacterial cell comprises a gene encoding a payload expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in Table 3 below. Underlined sequences are predicted ribosome binding sites, and bolded sequences are restriction sites used for cloning.

TABLE 3
FNR Promoter Sequences

| FNR Responsive | |
|---|---|
| Promoter | Sequence |
| SEQ ID NO: 1A | GTCAGCATAACACCCTGACCTCTCATTAATTGTTCATGCCGGGCG |
| | GCACTATCGTCGTCCGGCCTTTTCCTCTCTTACTCTGCTACGTAC |
| | ATCTATTTCTATAAATCCGTTCAATTTGTCTGTTTTTTGCACAAAC |
| | ATGAAATATCAGACAATTCCGTGACTTAAGAAAATTTATACAAA |
| | TCAGCAATATACCCCTTAAGGAGTATATAAAGGTGAATTTGATT |
| | TACATCAATAAGCGGGGTTGCTGAATCGTTAAGGTAGGCGGTAA |
| | TAGAAAAGAAATCGAGGCAAAA |
| SEQ ID NO: 2A | ATTTCCTCTCATCCCATCCGGGGTGAGAGTCTTTTCCCCCGACTT |
| | ATGGCTCATGCATGCATCAAAAAAGATGTGAGCTTGATCAAAAA |
| | CAAAAAATATTTCACTCGACAGGAGTATTTATATTGCGCCCGTT |
| | ACGTGGGCTTCGACTGTAAATCAGAAAGGAGAAAACACCT |
| SEQ ID NO: 3A | GTCAGCATAACACCCTGACCTCTCATTAATTGTTCATGCCGGGCG |
| | GCACTATCGTCGTCCGGCCTTTTCCTCTCTTACTCTGCTACGTAC |
| | ATCTATTTCTATAAATCCGTTCAATTTGTCTGTTTTTTGCACAAAC |
| | ATGAAATATCAGACAATTCCGTGACTTAAGAAAATTTATACAAA |
| | TCAGCAATATACCCCTTAAGGAGTATATAAAGGTGAATTTGATT |
| | TACATCAATAAGCGGGGTTGCTGAATCGTTAAGGATCCCTCTAG |
| | AAATAATTTTGTTTAACTTTAAGAAGGAGATATACAT |
| SEQ ID NO: 4A | CATTTCCTCTCATCCCATCCGGGGTGAGAGTCTTTTCCCCCGACT |
| | TATGGCTCATGCATGCATCAAAAAAGATGTGAGCTTGATCAAAA |
| | ACAAAAAATATTTCACTCGACAGGAGTATTTATATTGCGCCCGG |
| | ATCCCTCTAGAAATAATTTTGTTTAACTTTAAGAAGGAGATATA |
| | CAT |
| SEQ ID NO: 5A | AGTTGTTCTTATTGGTGGTGTTGCTTTATGGTTGCATCGTAGTAA |
| | ATGGTTGTAACAAAAGCAATTTTTCCGGCTGTCTGTATACAAAA |
| | ACGCCGTAAAGTTTGAGCGAAGTCAATAAACTCTCTACCCATTC |
| | AGGGCAATATCTCTCTTGGATCCCTCTAGAAATAATTTTGTTTA |
| | ACTTTAAGAAGGAGATATACAT |
| SEQ ID NO: 6A | ATCCCCATCACTCTTGATGGAGATCAATTCCCCAAGCTGCTAGAG |
| | CGTTACCTTGCCCTTAAACATTAGCAATGTCGATTTATCAGAGGG |
| | CCGACAGGCTCCCACAGGAGAAAACCG |
| SEQ ID NO: 7A | CTCTTGATCGTTATCAATTCCCACGCTGTTTCAGAGCGTTACCTT |
| | GCCCTTAAACATTAGCAATGTCGATTTATCAGAGGGCCGACAGG |
| | CTCCCACAGGAGAAAACCG |
| nirB1 | GTCAGCATAACACCCTGACCTCTCATTAATTGTTCATGCCGGGCG |
| SEQ ID NO: 8A | GCACTATCGTCGTCCGGCCTTTTCCTCTCTTACTCTGCTACGTACA |
| | TCTATTTCTATAAATCCGTTCAATTTGTCTGTTTTTTGCACAAACA |
| | TGAAATATCAGACAATTCCGTGACTTAAGAAAATTTATACAAAT |
| | CAGCAATATACCCCTTAAGGAGTATATAAAGGTGAATTTGATTT |
| | ACATCAATAAGCGGGGTTGCTGAATCGTTAAGGTAGGCGGTAAT |
| | AGAAAAGAAATCGAGGCAAAA |
| nirB2 | CGGCCCGATCGTTGAACATAGCGGTCCGCAGGCGGCACTGCTTA |
| SEQ ID NO: 9A | CAGCAAACGGTCTGTACGCTGTCGTCTTTGTGATGTGCTTCCTGT |
| | TAGGTTTCGTCAGCCGTCACCGTCAGCATAACACCCTGACCTCTC |
| | ATTAATTGCTCATGCCGGACGGCACTATCGTCGTCCGGCCTTTTC |
| | CTCTCTTCCCCCGCTACGTGCATCTATTTCTATAAACCCGCTCATT |
| | TTGTCTATTTTTTGCACAAACATGAAATATCAGACAATTCCGTGA |
| | CTTAAGAAAATTTATACAAATCAGCAATATACCCATTAAGGAGT |
| | ATATAAAGGTGAATTTGATTTACATCAATAAGCGGGGTTGCTGA |
| | ATCGTTAAGGTAGGCGGTAATAGAAAAGAAATCGAGGCAAAAat |
| | gtttgtttaactttaagaaggagatatacat |
| nirB3 | GTCAGCATAACACCCTGACCTCTCATTAATTGCTCATGCCGGACG |
| SEQ ID NO: 10A | GCACTATCGTCGTCCGGCCTTTTCCTCTCTTCCCCCGCTACGTGC |
| | ATCTATTTCTATAAACCCGCTCATTTTGTCTATTTTTTGCACAAAC |
| | ATGAAATATCAGACAATTCCGTGACTTAAGAAAATTTATACAAA |
| | TCAGCAATATACCCATTAAGGAGTATATAAAGGTGAATTTGATT |
| | TACATCAATAAGCGGGGTTGCTGAATCGTTAAGGTAGGCGGTAA |
| | TAGAAAAGAAATCGAGGCAAAA |
| ydfZ | ATTTCCTCTCATCCCATCCGGGGTGAGAGTCTTTTCCCCCGACTT |
| SEQ ID NO: 11A | ATGGCTCATGCATGCATCAAAAAAGATGTGAGCTTGATCAAAAA |
| | CAAAAAATATTTCACTCGACAGGAGTATTTATATTGCGCCCGTTA |
| | CGTGGGCTTCGACTGTAAATCAGAAAGGAGAAAACACCT |
| nirB + RBS | GTCAGCATAACACCCTGACCTCTCATTAATTGTTCATGCCGGGCG |
| SEQ ID NO: 12A | GCACTATCGTCGTCCGGCCTTTTCCTCTCTTACTCTGCTACGTACA |
| | TCTATTTCTATAAATCCGTTCAATTTGTCTGTTTTTTGCACAAACA |
| | TGAAATATCAGACAATTCCGTGACTTAAGAAAATTTATACAAAT |
| | CAGCAATATACCCCTTAAGGAGTATATAAAGGTGAATTTGATTT |
| | ACATCAATAAGCGGGGTTGCTGAATCGTTAAGGATCCCTCTAGA |
| | AATAATTTTGTTTAACTTTAAGAAGGAGATATACAT |
| ydfZ + RBS | CATTTCCTCTCATCCCATCCGGGGTGAGAGTCTTTTCCCCCGACT |
| SEQ ID NO: 13A | TATGGCTCATGCATGCATCAAAAAAGATGTGAGCTTGATCAAAA |
| | ACAAAAAATATTTCACTCGACAGGAGTATTTATATTGCGCCCGG |
| | ATCCCTCTAGAAATAATTTTGTTTAACTTTAAGAAGGAGATATA |
| | CAT |
| fnrS1 | AGTTGTTCTTATTGGTGGTGTTGCTTTATGGTTGCATCGTAGTAA |
| SEQ ID NO: 14A | ATGGTTGTAACAAAAGCAATTTTTCCGGCTGTCTGTATACAAAA |
| | ACGCCGTAAAGTTTGAGCGAAGTCAATAAACTCTCTACCCATTC |
| | AGGGCAATATCTCTCTTGGATCCCTCTAGAAATAATTTTGTTTAA |
| | CTTTAAGAAGGAGATATACAT |
| fnrS2 | AGTTGTTCTTATTGGTGGTGTTGCTTTATGGTTGCATCGTAGTAA |
| SEQ ID NO: 15A | ATGGTTGTAACAAAAGCAATTTTTCCGGCTGTCTGTATACAAAA |
| | ACGCCGCAAAGTTTGAGCGAAGTCAATAAACTCTCTACCCATTC |
| | AGGGCAATATCTCTCTTGGATCCAAAGTGAACTCTAGAAATAAT |
| | TTTGTTTAACTTTAAGAAGGAGATATACAT |
| nirB + crp | TCGTCTTTGTGATGTGCTTCCTGTTAGGTTTCGTCAGCCGTCACC |
| SEQ ID NO: 16A | GTCAGCATAACACCCTGACCTCTCATTAATTGCTCATGCCGGACG |
| | GCACTATCGTCGTCCGGCCTTTTCCTCTCTTCCCCCGCTACGTGC |
| | ATCTATTTCTATAAACCCGCTCATTTTGTCTATTTTTTGCACAAAC |
| | ATGAAATATCAGACAATTCCGTGACTTAAGAAAATTTATACAAA |
| | TCAGCAATATACCCATTAAGGAGTATATAAAGGTGAATTTGATT |
| | TACATCAATAAGCGGGGTTGCTGAATCGTTAAGGTAGaaatgtgatcta |
| | gttcacatttGCGGTAATAGAAAAGAAATCGAGGCAAAAatgtttgtttaacttta |
| | agaaggagatatacat |
| fnrS + crp | AGTTGTTCTTATTGGTGGTGTTGCTTTATGGTTGCATCGTAGTAA |
| SEQ ID NO: 17A | ATGGTTGTAACAAAAGCAATTTTTCCGGCTGTCTGTATACAAAA |
| | ACGCCGCAAAGTTTGAGCGAAGTCAATAAACTCTCTACCCATTC |
| | AGGGCAATATCTCTCaaatgtgatctagttcacattttttgtttaactttaagaaggagatatac |
| | at |

FNR promoter sequences are known in the art, and any suitable FNR promoter sequence(s) may be used in the genetically engineered bacteria of the invention. Any suitable FNR promoter(s) may be combined with any suitable payload.

Non-limiting FNR promoter sequences are provided in Table 3, which depicts the nucleic acid sequences of exemplary regulatory region sequences comprising a FNR-responsive promoter sequence. Underlined sequences are predicted ribosome binding sites, and bolded sequences are restriction sites used for cloning. In some embodiments, the genetically engineered bacteria of the invention comprise one or more of: SEQ ID NO: 1A, SEQ ID NO: 2A, SEQ ID NO: 3A, SEQ ID NO: 4A, SEQ ID NO: 5A, SEQ ID NO: 6A, SEQ ID NO: 7A, nirBi1 promoter (SEQ ID NO: 8A), nirB2 promoter (SEQ ID NO: 9A), nirB3 promoter (SEQ ID NO: 10A), ydfZ promoter (SEQ ID NO: 11A), nirB promoter fused to a strong ribosome binding site (SEQ ID NO: 12A), ydfZ promoter fused to a strong ribosome binding site (SEQ ID NO: 13A), fnrS, an anaerobically induced small RNA gene (fnrS1 promoter SEQ ID NO: 14A or fnrS2 promoter SEQ ID NO: 15A), nirB promoter fused to a crp binding site (SEQ ID NO: 16A), and fnrS fused to a crp binding site (SEQ ID NO: 17A). In some embodiments, the FNR-responsive promoter is at least about 80%, at least about 85%, at least about 90%, at least about 95%, or at least about 99% homologous to the sequence of any one of SEQ ID NOs: 1A-17A.

In some embodiments, multiple distinct FNR nucleic acid sequences are inserted in the genetically engineered bacteria. In alternate embodiments, the genetically engineered bacteria comprise a gene encoding a payload (e.g. PME e.g. PAL) expressed under the control of an alternate oxygen level-dependent promoter, e.g., DNR (Trunk et al., 2010) or ANR (Ray et al., 1997). In these embodiments, expression of the payload gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

Any suitable FNR promoter(s) may be combined with any suitable PAL. Non-limiting FNR promoter sequences are provided in Table 3, and non-limiting PAL sequences are also provided herein. In some embodiments, the genetically engineered bacteria of the invention comprise one or more of the following SEQ ID NOs disclosed in WO2017087580, the contents of which are herein incorporated by reference in their entirety: SEQ ID NO: 9, SEQ ID NO: 10, nirB1 promoter (SEQ ID NO: 11), nirB2 promoter (SEQ ID NO: 12), nirB3 promoter (SEQ ID NO: 13), ydfZ promoter (SEQ ID NO: 14), nirB promoter fused to a strong ribosome binding site (SEQ ID NO: 15), ydfZ promoter fused to a strong ribosome binding site (SEQ ID NO: 16), fnrS, an anaerobically induced small RNA gene (fnrS1 promoter SEQ ID NO: 9 or fnrS2 promoter SEQ ID NO: 17), nirB promoter fused to a crp binding site (SEQ ID NO: 18), andffnrS fused to a crp binding site (SEQ ID NO: 19).

In another embodiment, the genetically engineered bacteria comprise the gene or gene cassette for producing the payload expressed under the control of anaerobic regulation of arginine deiminiase and nitrate reduction transcriptional regulator (ANR). In P. aeruginosa, ANR is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT-ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonasfluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

The FNR family also includes the dissimilatory nitrate respiration regulator (DNR) (Arai et al., 1995), a transcriptional regulator which is required in conjunction with ANR for “anaerobic nitrate respiration of Pseudomonas aeruginosa” (Hasegawa et al., 1998). For certain genes, the FNR-binding motifs “are probably recognized only by DNR” (Hasegawa et al., 1998). Any suitable transcriptional regulator that is controlled by exogenous environmental conditions and corresponding regulatory region may be used. Non-limiting examples include ArcA/B, ResD/E, NreA/B/C, and AirSR, and others are known in the art.

In other embodiments, the one or more gene sequence(s) for producing a payload (e.g. a PME e.g. PAL) are expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Görke and Stilke, 2008). In some embodiments, the gene or gene cassette for producing a payload molecule is controlled by an oxygen level-dependent promoter fused to a CRP binding site. In some embodiments, the one or more gene sequence(s) for a payload are controlled by an FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and transcription of the gene or gene cassette for producing a payload is repressed. In some embodiments, an oxygen level-dependent promoter (e.g., an FNR promoter) fused to a binding site for a transcriptional activator is used to ensure that the gene or gene cassette for producing a payload is not expressed under anaerobic conditions when sufficient amounts of glucose are present, e.g., by adding glucose to growth media in vitro.

In some embodiments, the genetically engineered bacteria comprise an oxygen level-dependent promoter from a different species, strain, or substrain of bacteria. In some embodiments, the genetically engineered bacteria comprise an oxygen level-sensing transcription factor, e.g., FNR, ANR or DNR, from a different species, strain, or substrain of bacteria. In some embodiments, the genetically engineered bacteria comprise an oxygen level-sensing transcription factor and corresponding promoter from a different species, strain, or substrain of bacteria. The heterologous oxygen-level dependent transcriptional regulator and/or promoter increases the transcription of genes operably linked to said promoter, e.g., one or more gene sequence(s) for producing the payload(s) in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the payload, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the payload, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006). In some embodiments, both the oxygen level-sensing transcriptional regulator and corresponding promoter are mutated relative to the wild-type sequences from bacteria of the same subtype in order to increase expression of the payload in low-oxygen conditions.

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the payload are present on different plasmids. In some embodiments, the gene encoding the oxygen level-sensing transcriptional regulator and the gene encoding the payload are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the payload are present on different chromosomes. In some embodiments, the gene encoding the oxygen level-sensing transcriptional regulator and the gene encoding the payload are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the payload. In some embodiments, expression of the transcriptional regulator is controlled by the same promoter that controls expression of the payload. In some embodiments, the transcriptional regulator and the payload are divergently transcribed from a promoter region.

### Oxygen Level Independent Inducible Promoters

Oxygen Level Independent Inducible Promoters systems, such as systems including FNRS24Y, are described in PCT/US2016/062369, filed Nov. 16, 2016 and published as WO2017087580, the contents of which is herein incorporated by reference in its entirety.

In addition to promoters that are induced in response to oxygen levels, the PME gene(s) and/or Phe transporter gene(s) can be regulated by promoters that are induced in response to inflammatory conditions, such as in presence of reactive nitrogen species or in the presence of reactive oxygen species. Examples of such inducible promoters are found in co-pending, co-owned International Application PCT/US2016/050836, filed Sep. 8, 2016, the contents of which are hereby incorporated by reference in their entirety.

In any of the embodiments described herein, the genetically engineered bacteria comprising one or more PME and/or one or more phe transporters under control of an oxygen independent promoter further comprise one or more bacteriophages. In some embodiments, the bacteriophages have been mutated in one or more genes within the bacteriophage genome. Such mutations include deletions, insertions, substitutions and inversions and are located in or encompass one or more bacteriophage genes.

In some embodiments, the genetically engineered bacteria comprise one or more E. coli In some embodiments, the mutation is a deletion. In some embodiments, the genetically engineered bacteria comprise one or more deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### RNS-Dependent Regulation

In some embodiments, the genetically engineered bacteria or genetically engineered virus comprise a gene encoding a payload that is expressed under the control of an inducible promoter. In some embodiments, the genetically engineered bacterium or genetically engineered virus that expresses a payload under the control of a promoter that is activated by inflammatory conditions. In one embodiment, the gene for producing the payload is expressed under the control of an inflammatory-dependent promoter that is activated in inflammatory environments, e.g., a reactive nitrogen species or RNS promoter. In some embodiments, the genetically engineered bacteria of the invention comprise a tunable regulatory region that is directly or indirectly controlled by a transcription factor that is capable of sensing at least one reactive nitrogen species. Suitable RNS inducible promoters, e.g., inducible by reactive nitrogen species are described in International Patent Application PCT/US2016/062369, filed Nov. 16, 2016 and published as WO2017087580, published as WO2017/123675, the contents of which is herein incorporated by reference in its entirety.

### ROS-Dependent Regulation

In some embodiments, the genetically engineered bacteria or genetically engineered virus comprise a gene for producing a payload that is expressed under the control of an inducible promoter. In some embodiments, the genetically engineered bacterium or genetically engineered virus that expresses a payload under the control of a promoter that is activated by conditions of cellular damage. In one embodiment, the gene for producing the payload is expressed under the control of a cellular damaged-dependent promoter that is activated in environments in which there is cellular or tissue damage, e.g., a reactive oxygen species or ROS promoter. In some embodiments, the genetically engineered bacteria of the invention comprise a tunable regulatory region that is directly or indirectly controlled by a transcription factor that is capable of sensing at least one reactive oxygen species. Suitable ROS inducible promoters, e.g., inducible by reactive oxygen species are described in International Patent Application PCT/US2017/013072, filed Jan. 11, 2017, published as WO2017/123675, International Patent Applications PCT/US2016/032562, filed—May 13, 2016, published as WO2016183531, and PCT/US2016/062369, filed Nov. 16, 2016 and published as WO2017087580, the contents of each of which are herein incorporated by reference in their entireties.

TABLE 17
Nucleotide sequences of exemplary OxyR-regulated regulatory regions

| Regulatory | |
|---|---|
| Sequence | Sequence |
| katG | TGTGGCTTTTATGAAAATCACACAGTGATCACAAATTTTAAACAGAGC |
| (SEQ ID | ACAAAATGCTGCCTCGAAATGAGGGCGGGAAAATAAGGTTATCAGCC |
| NO: 18C) | TTGTTTTCTCCCTCATTACTTGAAGGATATGAAGCTAAAACCCTTTTTT |
| | ATAAAGCATTTGTCCGAATTCGGACATAATCAAAAAAGCTTAATTAAG |
| | ATCAATTTGATCTACATCTCTTTAACCAACAATATGTAAGATCTCAACT |
| | ATCGCATCCGTGGATTAATTCAATTATAACTTCTCTCTAACGCTGTGTA |
| | TCGTAACGGTAACACTGTAGAGGGGAGCACATTGATGCGAATTCATTA |
| | AAGAGGAGAAAGGTACC |
| dps | TTCCGAAAATTCCTGGCGAGCAGATAAATAAGAATTGTTCTTATCAAT |
| (SEQ ID | ATATCTAACTCATTGAATCTTTATTAGTTTTGTTTTTCACGCTTGTTACC |
| NO: 19C) | ACTATTAGTGTGATAGGAACAGCCAGAATAGCGGAACACATAGCCGG |
| | TGCTATACTTAATCTCGTTAATTACTGGGACATAACATCAAGAGGATA |
| | TGAAATTCGAATTCATTAAAGAGGAGAAAGGTACC |
| ahpC | GCTTAGATCAGGTGATTGCCCTTTGTTTATGAGGGTGTTGTAATCCATG |
| (SEQ ID | TCGTTGTTGCATTTGTAAGGGCAACACCTCAGCCTGCAGGCAGGCACT |
| NO: 20C) | GAAGATACCAAAGGGTAGTTCAGATTACACGGTCACCTGGAAAGGGG |
| | GCCATTTTACTTTTTATCGCCGCTGGCGGTGCAAAGTTCACAAAGTTGT |
| | CTTACGAAGGTTGTAAGGTAAAACTTATCGATTTGATAATGGAAACGC |
| | ATTAGCCGAATCGGCAAAAATTGGTTACCTTACATCTCATCGAAAACA |
| | CGGAGGAAGTATAGATGCGAATTCATTAAAGAGGAGAAAGGTACC |
| oxyS | CTCGAGTTCATTATCCATCCTCCATCGCCACGATAGTTCATGGCGATA |
| (SEQ ID | GGTAGAATAGCAATGAACGATTATCCCTATCAAGCATTCTGACTGATA |
| NO: 21C) | ATTGCTCACACGAATTCATTAAAGAGGAGAAAGGTACC |

In some embodiments, the regulatory region sequence is at least about 80%, at least about 85%, at least about 90%, at least about 95%, or at least about 99% homologous to the sequence of SEQ ID NO: 18C, SEQ ID NO: 19C, SEQ ID NO: 20C, and/or SEQ ID NO: 21C.

### Propionate and Other Promoters

In some embodiments, the genetically engineered bacteria comprise the gene or gene cassette for producing one or more payload genes expressed under the control of an inducible promoter that is responsive to specific molecules or metabolites in the environment, e.g., the tumor microenvironment, a specific tissue, or the mammalian gut. For example, the short-chain fatty acid propionate is a major microbial fermentation metabolite localized to the gut (Hosseini et al., 2011). In one embodiment, the gene or gene cassette for producing a payload is under the control of a propionate-inducible promoter. In a more specific embodiment, the gene or gene cassette for producing the payload is under the control of a propionate-inducible promoter that is activated by the presence of propionate in the mammalian gut. Any molecule or metabolite found in the mammalian gut, in a healthy and/or disease state, may be used to induce payload expression. Non-limiting examples of inducers include propionate, bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese. In alternate embodiments, the gene or gene cassette for producing therapeutic polypeptide is under the control of a pAraBAD promoter, which is activated in the presence of the sugar arabinose.

In some embodiments, the gene or gene cassette for producing the polypeptide of interest is present on a plasmid and operably linked to a promoter that is induced under low-oxygen or anaerobic conditions. In some embodiments, the gene or gene cassette for producing polypeptide of interest is present in the chromosome and operably linked to a promoter that is induced under low-oxygen or anaerobic conditions. In some embodiments, the gene or gene cassette for producing a polypeptide of interest is present on a plasmid and operably linked to a promoter that is induced by molecules or metabolites that are specific to the to the tumor and/or the mammalian gut. In some embodiments, the gene or gene cassette for producing polypeptide of interest is present on a chromosome and operably linked to a promoter that is induced by molecules or metabolites that are specific to the tumor and/or the mammalian gut. In some embodiments, the gene or gene cassette for producing polypeptide of interest is present on a chromosome and operably linked to a promoter that is induced by exposure to tetracycline. In some embodiments, the gene or gene cassette for producing polypeptide of interest is present on a plasmid and operably linked to a promoter that is induced by exposure to tetracycline.

In some embodiments, gene expression is further optimized by methods known in the art, e.g., by optimizing ribosomal binding sites (RBS), manipulating transcriptional regulators, and/or increasing mRNA stability. Bioinformatics tools for the fine tuning and optimization of RBS are known in the art.

In any of the embodiments described herein above (and elsewhere herein), the engineered bacteria may additionally comprise gene sequence(s) encoding one or more gene sequence(s) under the control of any of the promoters discussed herein. In some embodiments, the genetically engineered bacteria comprise a stably maintained plasmid or chromosome carrying the gene or gene cassette for producing the polypeptide of interest, such that the gene or gene cassette can be expressed in the host cell, and the host cell is capable of survival and/or growth in vitro, e.g., in medium, and/or in vivo, e.g., in the gut or the tumor microenvironment. In some embodiments, a bacterium may comprise multiple copies of the gene or gene cassette for producing a polypeptide of interest. In some embodiments, gene or gene cassette for producing the payload is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, gene or gene cassette for producing a polypeptide of interest is expressed on a high-copy plasmid. In some embodiments, the high-copy plasmid may be useful for increasing gene or gene cassette expression. In some embodiments, gene or gene cassette for producing a polypeptide of interest is expressed on a chromosome.

### Other Inducible Promoters

In some embodiments, the gene encoding a polypeptide of interest is present on a plasmid and operably linked to a promoter that is induced by one or more nutritional and/or chemical inducer(s) and/or metabolite(s). In some embodiments, the gene encoding a polypeptide of interest is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding a polypeptide of interest, such that a polypeptide of interest can be expressed in the host cell, and the host cell is capable of survival and/or growth in vitro, e.g., in medium, and/or in vivo, e.g., in the tumor or in the gut. In some embodiments, bacterial cell comprises two or more distinct copies of the one or more gene sequences(s) encoding a polypeptide of interest, which is controlled by a promoter inducible one or more nutritional and/or chemical inducer(s) and/or metabolite(s). In some embodiments, the genetically engineered bacteria comprise multiple copies of the one or more gene sequences(s) encoding a polypeptide of interest, which is controlled by a promoter inducible by one or more nutritional and/or chemical inducer(s) and/or metabolite(s). In some embodiments, the one or more gene sequences(s) encoding a polypeptide of interest(s), is present on a plasmid and operably linked to a directly or indirectly inducible promoter inducible by one or more nutritional and/or chemical inducer(s) and/or metabolite(s). In some embodiments, the one or more gene sequences(s) encoding a polypeptide of interest, is present on a chromosome and operably linked to a directly or indirectly inducible promoter. In some embodiments, the one or more gene sequence(s) encoding a polypeptide of interest is induced by one or more nutritional and/or chemical inducer(s) and/or metabolites.

In some embodiments, one or more gene sequence(s) encoding polypeptides of interest described herein is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding a polypeptide of interest, which is induced by one or more nutritional and/or chemical inducer(s) and/or metabolite(s), such that a polypeptide of interest can be expressed in the host cell, and the host cell is capable of survival and/or growth in vitro, e.g., under culture conditions, and/or in vivo, e.g., in the gut or the tumor microenvironment. In some embodiments, bacterial cell comprises two or more gene sequence(s) for the production of a polypeptide of interest, one or more of which are induced by one or more nutritional and/or chemical inducer(s) and/or metabolite(s). In some embodiments, the genetically engineered bacteria comprise multiple copies of the same gene sequence(s) for the production of a polypeptide of interest which are induced by one or more nutritional and/or chemical inducer(s) and/or metabolite(s). In some embodiments, the genetically engineered bacteria comprise multiple copies of different gene sequence(s) for the production of a polypeptide of interest, one or more of which are induced by one or more nutritional and/or chemical inducer(s) and/or metabolite(s).

In some embodiments, the gene sequence(s) for the production of a polypeptide of interest is present on a plasmid and operably linked to a promoter that is induced by one or more nutritional and/or chemical inducer(s) and/or metabolite(s). In some embodiments, gene sequence(s) for the production of a polypeptide of interest is present in the chromosome and operably linked to a promoter that is induced by one or more nutritional and/or chemical inducer(s) and/or metabolite(s).

In some embodiments, the genetically engineered bacteria comprise two or more distinct PAL genes. In some embodiments, the genetically engineered bacteria comprise multiple copies of the same PAL gene. In some embodiments, the PAL gene is present on a plasmid and operably linked to a directly or indirectly inducible promoter. In some embodiments, the PAL gene is present on a plasmid and operably linked to a promoter that is induced under low-oxygen or anaerobic conditions. In some embodiments, the PAL gene is present on a chromosome and operably linked to a directly or indirectly inducible promoter. In some embodiments, the PAL gene is present in the chromosome and operably linked to a promoter that is induced under low-oxygen or anaerobic conditions. In some embodiments, the PAL gene is present on a plasmid and operably linked to a promoter that is induced by exposure to tetracycline. In some embodiments, the PAL gene is present on a plasmid and operably linked to a promoter that is induced by exposure to arabinose. In some embodiments, the PAL gene is present on a plasmid and operably linked to a promoter that is induced by exposure to IPTG or another LacI inducer. In some embodiments, the PAL gene is present on a plasmid and operably linked to a promoter that is induced by exposure to rhamnose. In some embodiments, the PAL gene is present on a plasmid and operably linked to a promoter that is induced by exposure to tetracycline. In some embodiments, the PAL gene is present on a plasmid and operably linked to a promoter that is induced by change in temperature from a non-permissive temperature to a permissive temperature. In some embodiments, the PAL gene is present on a chromosome and operably linked to a promoter that is induced by exposure to arabinose. In some embodiments, the PAL gene is present on a chromosome and operably linked to a promoter that is induced by exposure to IPTG or another LacI inducer. In some embodiments, the PAL gene is present on a chromosome and operably linked to a promoter that is induced by exposure to rhamnose. In some embodiments, the PAL gene is present on a chromosome and operably linked to a promoter that is induced by exposure to tetracycline. In some embodiments, the PAL gene is present on a chromosome and operably linked to a promoter that is induced by change in temperature from a non-permissive temperature to a permissive temperature.

In some embodiments, the genetically engineered bacteria comprise a stably maintained plasmid or chromosome carrying the LAAD gene, such that LAAD can be expressed in the host cell, and the host cell is capable of survival and/or growth in vitro, e.g., in medium, and/or in vivo, e.g., in the gut. In some embodiments, the genetically engineered bacteria comprise two or more distinct LAAD genes. In some embodiments, the genetically engineered bacteria comprise multiple copies of the same LAAD gene. In some embodiments, the LAAD gene is present on a plasmid and operably linked to a directly or indirectly inducible promoter. In some embodiments, the LAAD gene is present on a plasmid and operably linked to a promoter that is inducible, e.g., by arabinose or tetracycline. In some embodiments, the LAAD gene is present on a chromosome and operably linked to a directly or indirectly inducible promoter. In some embodiments, the LAAD gene is present in the chromosome and operably linked to a promoter that is induced, e.g., by arabinose or tetracycline. In some embodiments, the LAAD gene is present on a plasmid and operably linked to a promoter that is induced by exposure to tetracycline. In some embodiments, the LAAD gene is present on a plasmid and operably linked to a promoter that is induced by exposure to arabinose. In some embodiments, the LAAD gene is present on a plasmid and operably linked to a promoter that is induced by exposure to IPTG or another LacI inducer. In some embodiments, the LAAD gene is present on a plasmid and operably linked to a promoter that is induced by exposure to rhamnose. In some embodiments, the LAAD gene is present on a plasmid and operably linked to a promoter that is induced by change in temperature from a non-permissive temperature to a permissive temperature. In some embodiments, the LAAD gene is present on a plasmid and operably linked to a constitutive promoter. In some embodiments, the LAAD gene is present on a plasmid and operably linked to a promoter that is induced by exposure to tetracycline. In some embodiments, the LAAD gene is present on a chromosome and operably linked to a promoter that is induced by exposure to arabinose. In some embodiments, the LAAD gene is present on a chromosome and operably linked to a promoter that is induced by exposure to IPTG or another LacI inducer. In some embodiments, the LAAD gene is present on a chromosome and operably linked to a promoter that is induced by exposure to rhamnose. In some embodiments, the LAAD gene is present on a chromosome and operably linked to a promoter that is induced by change in temperature from a non-permissive temperature to a permissive temperature. In some embodiments, the LAAD gene is present on a chromosome and operably linked to a constitutive promoter.

In any of these embodiments of bacteria comprising PME gene(s), e.g., PAL, PAH, and/or LAAD, the bacteria may further comprise gene sequence encoding one or more Phe transporters, which Phe transporter gene sequence(s) may be present on a plasmid or chromosome, which may be the same or a different plasmid or chromosome from the location of the PME gene. The Phe transporter gene sequence(s) may be under the control of the same or a different promoter from the PMR gene sequence(s).

In some embodiments, the genetically engineered bacteria comprise an oxygen-level dependent transcriptional regulator, e.g., FNR, ANR, or DNR, and corresponding promoter from a different bacterial species. The non-native oxygen-level dependent transcriptional regulator and promoter increase the transcription of genes operably linked to said promoter, e.g., PAL or PAH, in a low-oxygen or anaerobic environment, as compared to the native transcriptional regulator and promoter in the bacteria under the same conditions. PAL or PAH, in a low-oxygen or anaerobic environment, as compared to the wild-type promoter under the same conditions. PAL or PAH, in a low-oxygen or anaerobic environment, as compared to the wild-type transcriptional regulator under the same conditions. 2006).

In some embodiments, the genetically engineered bacteria of the invention comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding PAL are present on different plasmids. In some embodiments, the gene encoding the oxygen level-sensing transcriptional regulator and the gene encoding PAL are present on the same plasmid. In some embodiments, the gene encoding the oxygen level-sensing transcriptional regulator is present on a chromosome. In some embodiments, the gene encoding the oxygen level-sensing transcriptional regulator and the gene encoding PAL are present on different chromosomes. In some embodiments, the gene encoding the oxygen level-sensing transcriptional regulator and the gene encoding PAL are present on the same chromosome.

In one embodiment, LAAD expression is under the control of the ParaBAD promoter. In one embodiment, expression of LAAD occurs under aerobic or microaerobic conditions. In one embodiment, PAL expression is under the control of the ParaBAD promoter. In one embodiment, PAL expression occurs under aerobic or microaerobic conditions. In one embodiment, PAL expression occurs under anaerobic or low oxygen conditions and LADD expression occurs under aerobic or microaerobic conditions. In one embodiment, PAL expression occurs under anaerobic or low oxygen conditions and LADD expression is under the control of the ParaBAD promoter.

In some embodiments, one or more gene(s) or gene cassette(s) for producing polypeptide(s) of interest (e.g., PAL and LAAD gene) are present, and each gene is expressed under the control of different promoters, such as any of the promoters discussed in this paragraph and elsewhere herein.

In some embodiments, the one or more PME genes, e.g., PAL and/or LAAD gene are expressed under the control of a promoter that is induced by exposure to arabinose. In some embodiments, the one or more PME genes, e.g., PAL and/or LAAD gene are expressed under the control of a promoter that is induced by exposure to IPTG or other LacI inducer. In some embodiments, the one or more PME genes, e.g., PAL and/or LAAD gene are expressed under the control of a promoter that is induced by exposure to rhamnose. In some embodiments, the one or more PME genes, e.g., PAL and/or LAAD gene are expressed under the control of a promoter that is induced by a change in temperature from a non-permissive temperature to a permissive temperature.

In some embodiments, the promoter that is operably linked to the gene encoding polypeptide of interest is directly or indirectly induced by one or more nutritional and/or chemical inducer(s) and/or metabolite(s).

In some embodiments, one or more inducible promoter(s) are useful for or induced during in vivo expression of the one or more protein(s) of interest. In some embodiments, the promoters are induced during in vivo expression of one or more anti-cancer, satiety, gut barrier enhancer, immune modulatory and/or neuromodulatory molecules and/or other polypeptide(s) of interest. In some embodiments, expression of one or more a polypeptide of interest(s) and/or other polypeptide(s) of interest is driven directly or indirectly by one or more arabinose inducible promoter(s) in vivo. In some embodiments, the promoter is directly or indirectly induced by a chemical and/or nutritional inducer and/or metabolite which is co-administered with the genetically engineered bacteria of the invention.

In some embodiments, expression of one or more a polypeptide of interest and/or other polypeptide(s) of interest, is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with a polypeptide of interest(s) and/or other polypeptide(s) of interest prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, expression of one or more polypeptides of interest, e.g., one or more therapeutic polypeptide(s), is driven directly or indirectly by one or more arabinose inducible promoter(s).

In some embodiments, the arabinose inducible promoter is useful for or induced during in vivo expression of the one or more protein(s) of interest. In some embodiments, expression of one or more protein(s) of interest is driven directly or indirectly by one or more arabinose inducible promoter(s) in vivo. In some embodiments, the promoter is directly or indirectly induced by a molecule that is co-administered with the genetically engineered bacteria of the invention, e.g., arabinose.

In some embodiments, expression of one or more protein(s) of interest, is driven directly or indirectly by one or more arabinose inducible promoter(s) during in vitro growth, preparation, or manufacturing of the strain prior to in vivo administration. In some embodiments, the arabinose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., arabinose. In some embodiments, the cultures, which are induced by arabinose, are grown aerobically. In some embodiments, the cultures, which are induced by arabinose, are grown anaerobically.

In one embodiment, the arabinose inducible promoter drives the expression of a construct comprising one or more protein(s) of interest, jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., arabinose and IPTG). In another non-limiting example, the first inducing conditions may be culture conditions, e.g., including arabinose presence, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, the arabinose inducible promoter drives the expression of one or more protein(s) of interest from a low-copy plasmid or a high copy plasmid or a biosafety system plasmid described herein. In some embodiments, the arabinose inducible promoter drives the expression of one or more protein(s) of interest from a construct which is integrated into the bacterial chromosome. Exemplary insertion sites are described herein.

In some embodiments, one or more protein(s) of interest are knocked into the arabinose operon and are driven by the native arabinose inducible promoter

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 23 of Table 18. In some embodiments, the arabinose inducible construct further comprises a gene encoding AraC, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 24 of Table 18. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 25 of Table 18.

In some embodiments, the genetically engineered bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein. In one embodiment, expression of one or more protein(s) of interest is driven directly or indirectly by one or more rhamnose inducible promoter(s). In one embodiment, expression of the payload is driven directly or indirectly by a rhamnose inducible promoter.

In some embodiments, the rhamnose inducible promoter is useful for or induced during in vivo expression of the one or more protein(s) of interest. In some embodiments, expression of one or more protein(s) of interest is driven directly or indirectly by one or more rhamnose inducible promoter(s) in vivo. In some embodiments, the promoter is directly or indirectly induced by a molecule that is co-administered with the genetically engineered bacteria of the invention, e.g., rhamnose

In some embodiments, expression of one or more protein(s) of interest, is driven directly or indirectly by one or more rhamnose inducible promoter(s) during in vitro growth, preparation, or manufacturing of the strain prior to in vivo administration. In some embodiments, the rhamnose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., rhamnose. In some embodiments, the cultures, which are induced by rhamnose, are grown aerobically. In some embodiments, the cultures, which are induced by rhamnose, are grown anaerobically.

In one embodiment, the rhamnose inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., rhamnose and arabinose). In another non-limiting example, the first inducing conditions may be culture conditions, e.g., including rhamnose presence, and the second inducing conditions may be in vivo conditions. Such in vivo conditions include low-oxygen, microaerobic, or anaerobic conditions, conditions of the tumor microenvironment, presence of gut metabolites, and/or metabolites administered in combination with the bacterial strain. In some embodiments, the one or more rhamnose promoters drive expression of one or more protein(s) of interest and/or transcriptional regulator(s), e.g., FNRS24Y, in combination with the FNR promoter driving the expression of the same gene sequence(s).

In some embodiments, the rhamnose inducible promoter drives the expression of one or more protein(s) of interest, from a low-copy plasmid or a high copy plasmid or a biosafety system plasmid described herein. In some embodiments, the rhamnose inducible promoter drives the expression of one or more protein(s) of interest, from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 26 of Table 18.

In some embodiments, the genetically engineered bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lacd) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate Lacd, can be used instead of IPTG in a similar manner.

In one embodiment, expression of one or more protein(s) of interest is driven directly or indirectly by one or more IPTG inducible promoter(s).

In some embodiments, the IPTG inducible promoter is useful for or induced during in vivo expression of the one or more protein(s) of interest. In some embodiments, expression of one or more protein(s) of interest is driven directly or indirectly by one or more IPTG inducible promoter(s) in vivo. In some embodiments, the promoter is directly or indirectly induced by a molecule that is co-administered with the genetically engineered bacteria of the invention, e.g., IPTG.

In some embodiments, expression of one or more protein(s) of interest is driven directly or indirectly by one or more IPTG inducible promoter(s) during in vitro growth, preparation, or manufacturing of the strain prior to in vivo administration. In some embodiments, the IPTG inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., IPTG. In some embodiments, the cultures, which are induced by IPTG, are grown arerobically. In some embodiments, the cultures, which are induced by IPTG, are grown anaerobically.

In one embodiment, the IPTG inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., arabinose and IPTG). In another non-limiting example, the first inducing conditions may be culture conditions, e.g., including IPTG presence, and the second inducing conditions may be in vivo conditions. Such in vivo conditions include low-oxygen, microaerobic, or anaerobic conditions, conditions of the tumor microenvironment, presence of gut metabolites, and/or metabolites administered in combination with the bacterial strain. In some embodiments, the one or more IPTG inducible promoters drive expression of one or more protein(s) of interest in combination with the FNR promoter driving the expression of the same gene sequence(s).

In some embodiments, the IPTG inducible promoter drives the expression of one or more protein(s) of interest from a low-copy plasmid or a high copy plasmid or a biosafety system plasmid described herein. In some embodiments, the IPTG inducible promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 27 of Table 18. In some embodiments, the IPTG inducible construct further comprises a gene encoding lacI, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 28 of Table 18. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 29 of Table 18.

In some embodiments, the genetically engineered bacteria comprise one or more gene sequence(s) which are inducible through a tetracycline inducible system. The initial system Gossen and Bujard (Tight control of gene expression in mammalian cells by tetracycline-responsive promoters. (Gossen M & Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of one or more protein(s) of interest is driven directly or indirectly by one or more tetracycline inducible promoter(s). In some embodiments, the tetracycline inducible promoter is useful for or induced during in vivo expression of the one or more protein(s) of interest. In some embodiments, expression of one or more protein(s) of interest and/or transcriptional regulator(s), e.g., FNRS24Y, is driven directly or indirectly by one or more tetracycline inducible promoter(s) in vivo. In some embodiments, the promoter is directly or indirectly induced by a molecule that is co-administered with the genetically engineered bacteria of the invention, e.g., tetracycline

In some embodiments, expression of one or more protein(s) of interest is driven directly or indirectly by one or more tetracycline inducible promoter(s) during in vitro growth, preparation, or manufacturing of the strain prior to in vivo administration. In some embodiments, the tetracycline inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., tetracycline. In some embodiments, the cultures, which are induced by tetracycline, are grown arerobically. In some embodiments, the cultures, which are induced by tetracycline, are grown anaerobically.

In one embodiment, the tetracycline inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., tetracycline and IPTG). In another non-limiting example, the first inducing conditions may be culture conditions, e.g., including tetracycline presence, and the second inducing conditions may be in vivo conditions. Such in vivo conditions include low-oxygen, microaerobic, or anaerobic conditions, conditions of the tumor microenvironment, presence of gut metabolites, and/or metabolites administered in combination with the bacterial strain. In some embodiments, the one or more tetracycline promoters drive expression of one or more protein(s) of interest in combination with the FNR promoter driving the expression of the same gene sequence(s).

In some embodiments, the tetracycline inducible promoter drives the expression of one or more protein(s) of interest from a low-copy plasmid or a high copy plasmid or a biosafety system plasmid described herein. In some embodiments, the tetracycline inducible promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the bolded sequences of SEQ ID NO: 34 (tet promoter is in bold) of Table 18. In some embodiments, the tetracycline inducible construct further comprises a gene encoding AraC, which is divergently transcribed from the same promoter as the one or more one or more protein(s) of interest In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 34 in italics (Tet repressor is in italics) of Table 18. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 34 in italics (Tet repressor is in italics) of Table 18.

In some embodiments, the genetically engineered bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The gene of interest cloned downstream of the λ promoters can then be efficiently regulated by the mutant thermolabile cI857 repressor of bacteriophage λ. At temperatures below 37° C., cI857 binds to the oL or oR regions of the pR promoter and blocks transcription by RNA polymerase. At higher temperatures, the functional cI857 dimer is destabilized, binding to the oL or oR DNA sequences is abrogated, and mRNA transcription is initiated. An exemplary construct is depicted in FIG. 88A of WO2017087580, the contents of which are herein incorporated by reference in their entirety. Inducible expression from the ParaBad can be controlled or further fine-tuned through the optimization of the ribosome binding site (RBS), as described herein.

In one embodiment, expression of one or more protein(s) of interest is driven directly or indirectly by one or more thermoregulated promoter(s). In some embodiments, thermoregulated promoter is useful for or induced during in vivo expression of the one or more protein(s) of interest. In some embodiments, expression of one or more protein(s) of interest is driven directly or indirectly by one or more thermoregulated promoter(s) in vivo. In some embodiments, the promoter is directly or indirectly induced by a molecule that is co-administered with the genetically engineered bacteria of the invention, e.g., temperature.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more protein(s) of interest. This can be done in a thermoregulated system by growing the strain at lower temperatures, e.g., 30 C. Expression can then be induced by elevating the temperature to 37 C and/or 42 C. In some embodiments, thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37 C and 42 C, are grown arerobically. In some embodiments, the cultures, which are induced by induced by temperatures between 37 C and 42 C, are grown anaerobically.

In one embodiment, thermoregulated promoter drives the expression of a construct comprising one or more protein(s) of interest jointly with a second promoter, e.g., a second constitutive or inducible promoter. In some embodiments, two promoters are positioned proximally to the construct and drive its expression, wherein thermoregulated promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., thermoregulation and arabinose). In another non-limiting example, the first inducing conditions may be culture conditions, e.g., permissive temperature, and the second inducing conditions may be in vivo conditions. Such in vivo conditions include low-oxygen, microaerobic, or anaerobic conditions, conditions of the tumor microenvironment, presence of gut metabolites, and/or metabolites administered in combination with the bacterial strain. In some embodiments, the one or more thermoregulated promoters drive expression of one or more protein(s) of interest in combination with the FNR promoter driving the expression of the same gene sequence(s).

In some embodiments, thermoregulated promoter drives the expression of one or more protein(s) of interest from a low-copy plasmid or a high copy plasmid or a biosafety system plasmid described herein. In some embodiments, thermoregulated promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 30 of Table 18. In some embodiments, thermoregulated construct further comprises a gene encoding mutant cI857 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 31 of Table 18. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 33 of Table 18.

In some embodiments, the genetically engineered bacteria comprise one or more gene sequence(s) which are indirectly inducible through a system driven by the PssB promoter. The Pssb promoter is active under aerobic conditions, and shuts off under anaerobic conditions.

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control. FIG. 89A of WO2017087580, the contents of which are herein incorporated by reference in their entirety depicts a schematic of the gene organization of a PssB promoter.

In one embodiment, expression of one or more protein(s) of interest is indirectly regulated by a repressor expressed under the control of one or more PssB promoter(s).

In some embodiments, induction of the PssB promoter(s) indirectly drives the in vivo expression of one or more protein(s) of interest. In some embodiments, induction of the PssB promoter(s) indirectly drives the expression of one or more protein(s) of interest during in vitro growth, preparation, or manufacturing of the strain prior to in vivo administration. In some embodiments, conditions for induction of the PssB promoter(s) are provided in culture, e.g., in a flask, fermenter or other appropriate culture vessel, e.g., used during cell growth, cell expansion, fermentation, recovery, purification, formulation, and/or manufacture.

In some embodiments, the PssB promoter indirectly drives the expression of one or more protein(s) of interest from a low-copy plasmid or a high copy plasmid or a biosafety system plasmid described herein. In some embodiments, the PssB promoter indirectly drives the expression of one or more protein(s) of interest from a construct which is integrated into the bacterial chromosome. Exemplary insertion sites are described herein.

In another non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut and/or conditions of the tumor microenvironment, but prevent survival under aerobic conditions (biosafety switch). In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 35 of Table 18.

Sequences useful for expression from inducible promoters are listed in Table 18.

TABLE 18
Inducible promoter construct sequences

| Description | Sequence |
|---|---|
| Arabinose | CAGACATTGCCGTCACTGCGTCTTTTACTGGCTCTTCTCGCTAAC |
| Promoter region | CCAACCGGTAACCCCGCTTATTAAAAGCATTCTGTAACAAAGCG |
| SEQ ID NO: 23 | GGACCAAAGCCATGACAAAAACGCGTAACAAAAGTGTCTATAA |
| | TCACGGCAGAAAAGTCCACATTGATTATTTGCACGGCGTCACAC |
| | TTTGCTATGCCATAGCATTTTTATCCATAAGATTAGCGGATCCA |
| | GCCTGACGCTTTTTTTCGCAACTCTCTACTGTTTCTCCATACCTC |
| | TAGAAATAATTTTGTTTAACTTTAAGAAGGAGATATACAT |
| AraC (reverse | TTATTCACAACCTGCCCTAAACTCGCTCGGACTCGCCCCGGTGC |
| orientation) | ATTTTTTAAATACTCGCGAGAAATAGAGTTGATCGTCAAAACCG |
| SEQ ID NO: 24 | ACATTGCGACCGACGGTGGCGATAGGCATCCGGGTGGTGCTCA |
| | AAAGCAGCTTCGCCTGACTGATGCGCTGGTCCTCGCGCCAGCTT |
| | AATACGCTAATCCCTAACTGCTGGCGGAACAAATGCGACAGAC |
| | GCGACGGCGACAGGCAGACATGCTGTGCGACGCTGGCGATATC |
| | AAAATTACTGTCTGCCAGGTGATCGCTGATGTACTGACAAGCCT |
| | CGCGTACCCGATTATCCATCGGTGGATGGAGCGACTCGTTAATC |
| | GCTTCCATGCGCCGCAGTAACAATTGCTCAAGCAGATTTATCGC |
| | CAGCAATTCCGAATAGCGCCCTTCCCCTTGTCCGGCATTAATGA |
| | TTTGCCCAAACAGGTCGCTGAAATGCGGCTGGTGCGCTTCATCC |
| | GGGCGAAAGAAACCGGTATTGGCAAATATCGACGGCCAGTTAA |
| | GCCATTCATGCCAGTAGGCGCGCGGACGAAAGTAAACCCACTG |
| | GTGATACCATTCGTGAGCCTCCGGATGACGACCGTAGTGATGA |
| | ATCTCTCCAGGCGGGAACAGCAAAATATCACCCGGTCGGCAGA |
| | CAAATTCTCGTCCCTGATTTTTCACCACCCCCTGACCGCGAATG |
| | GTGAGATTGAGAATATAACCTTTCATTCCCAGCGGTCGGTCGAT |
| | AAAAAAATCGAGATAACCGTTGGCCTCAATCGGCGTTAAACCC |
| | GCCACCAGATGGGCGTTAAACGAGTATCCCGGCAGCAGGGGAT |
| | CATTTTGCGCTTCAGCCATACTTTTCATACTCCCGCCATTCAGAG |
| | AAGAAACCAATTGTCCATATTGCAT |
| AraC polypeptide | MQYGQLVSSLNGGSMKSMAEAQNDPLLPGYSFNAHLVAGLTPIE |
| SEQ ID NO: 25 | ANGYLDFFIDRPLGMKGYILNLTIRGQGVVKNQGREFVCRPGDILL |
| | FPPGEIHHYGRHPEAHEWYHQWVYFRPRAYWHEWLNWPSIFANT |
| | GFFRPDEAHQPHFSDLFGQIINAGQGEGRYSELLAINLLEQLLLRRM |
| | EAINESLHPPMDNRVREACQYISDHLADSNFDIASVAQHVCLSPSR |
| | LSHLFRQQLGISVLSWREDQRISQAKLLLSTTRMPIATVGRNVGFD |
| | DQLYFSRVFKKCTGASPSEFRAGCE* |
| Region | CGGTGAGCATCACATCACCACAATTCAGCAAATTGTGAACATC |
| comprising | ATCACGTTCATCTTTCCCTGGTTGCCAATGGCCCATTTTCCTGTC |
| rhamnose | AGTAACGAGAAGGTCGCGAATCAGGCGCTTTTTAGACTGGTCG |
| inducible | TAATGAAATTCAGCTGTCACCGGATGTGCTTTCCGGTCTGATGA |
| promoter | GTCCGTGAGGACGAAACAGCCTCTACAAATAATTTTGTTTAAAA |
| SEQ ID NO: 26 | CAACACCCACTAAGATAACTCTAGAAATAATTTTGTTTAACTTT |
| | AAGAAGGAGATATACAT |
| Lac Promoter | ATTCACCACCCTGAATTGACTCTCTTCCGGGCGCTATCATGCCA |
| region | TACCGCGAAAGGTTTTGCGCCATTCGATGGCGCGCCGCTTCGTC |
| SEQ ID NO: 27 | AGGCCACATAGCTTTCTTGTTCTGATCGGAACGATCGTTGGCTG |
| | TGTTGACAATTAATCATCGGCTCGTATAATGTGTGGAATTGTGA |
| | GCGCTCACAATTAGCTGTCACCGGATGTGCTTTCCGGTCTGATG |
| | AGTCCGTGAGGACGAAACAGCCTCTACAAATAATTTTGTTTAAA |
| | ACAACACCCACTAAGATAACTCTAGAAATAATTTTGTTTAACTT |
| | TAAGAAGGAGATATACAT |
| LacO | GGAATTGTGAGCGCTCACAATT |
| LacI (in reverse | TCACTGCCCGCTTTCCAGTCGGGAAACCTGTCGTGCCAGCTGCA |
| orientation) | TTAATGAATCGGCCAACGCGCGGGGAGAGGCGGTTTGCGTATT |
| SEQ ID NO: 28 | GGGCGCCAGGGTGGTTTTTCTTTTCACCAGTGAGACTGGCAACA |
| | GCTGATTGCCCTTCACCGCCTGGCCCTGAGAGAGTTGCAGCAAG |
| | CGGTCCACGCTGGTTTGCCCCAGCAGGCGAAAATCCTGTTTGAT |
| | GGTGGTTAACGGCGGGATATAACATGAGCTATCTTCGGTATCGT |
| | CGTATCCCACTACCGAGATATCCGCACCAACGCGCAGCCCGGA |
| | CTCGGTAATGGCGCGCATTGCGCCCAGCGCCATCTGATCGTTGG |
| | CAACCAGCATCGCAGTGGGAACGATGCCCTCATTCAGCATTTGC |
| | ATGGTTTGTTGAAAACCGGACATGGCACTCCAGTCGCCTTCCCG |
| | TTCCGCTATCGGCTGAATTTGATTGCGAGTGAGATATTTATGCC |
| | AGCCAGCCAGACGCAGACGCGCCGAGACAGAACTTAATGGGCC |
| | CGCTAACAGCGCGATTTGCTGGTGACCCAATGCGACCAGATGCT |
| | CCACGCCCAGTCGCGTACCGTCCTCATGGGAGAAAATAATACT |
| | GTTGATGGGTGTCTGGTCAGAGACATCAAGAAATAACGCCGGA |
| | ACATTAGTGCAGGCAGCTTCCACAGCAATGGCATCCTGGTCATC |
| | CAGCGGATAGTTAATGATCAGCCCACTGACGCGTTGCGCGAGA |
| | AGATTGTGCACCGCCGCTTTACAGGCTTCGACGCCGCTTCGTTC |
| | TACCATCGACACCACCACGCTGGCACCCAGTTGATCGGCGCGA |
| | GATTTAATCGCCGCGACAATTTGCGACGGCGCGTGCAGGGCCA |
| | GACTGGAGGTGGCAACGCCAATCAGCAACGACTGTTTGCCCGC |
| | CAGTTGTTGTGCCACGCGGTTGGGAATGTAATTCAGCTCCGCCA |
| | TCGCCGCTTCCACTTTTTCCCGCGTTTTCGCAGAAACGTGGCTG |
| | GCCTGGTTCACCACGCGGGAAACGGTCTGATAAGAGACACCGG |
| | CATACTCTGCGACATCGTATAACGTTACTGGTTTCAT |
| LacI polypeptide | MKPVTLYDVAEYAGVSYQTVSRVVNQASHVSAKTREKVEAAMA |
| sequence | ELNYIPNRVAQQLAGKQSLLIGVATSSLALHAPSQIVAAIKSRADQ |
| SEQ ID NO: 29 | LGASVVVSMVERSGVEACKAAVHNLLAQRVSGLIINYPLDDQDAI |
| | AVEAACTNVPALFLDVSDQTPINSIIFSHEDGTRLGVEHLVALGHQ |
| | QIALLAGPLSSVSARLRLAGWHKYLTRNQIQPIAEREGDWSAMSG |
| | FQQTMQMLNEGIVPTAMLVANDQMALGAMRAITESGLRVGADIS |
| | VVGYDDTEDSSCYIPPLTTIKQDFRLLGQTSVDRLLQLSQGQAVKG |
| | NQLLPVSLVKRKTTLAPNTQTASPRALADSLMQLARQVSRLESGQ |
| Region | ACGTTAAATCTATCACCGCAAGGGATAAATATCTAACACCGTGC |
| comprising | GTGTTGACTATTTTACCTCTGGCGGTGATAATGGTTGCATAGCT |
| Temperature | GTCACCGGATGTGCTTTCCGGTCTGATGAGTCCGTGAGGACGAA |
| sensitive | ACAGCCTCTACAAATAATTTTGTTTAAAACAACACCCACTAAGA |
| promoter | TAACTCTAGAAATAATTTTGTTTAACTTTAAGAAGGAGATATAC |
| SEQ ID NO: 30 | AT |
| mutant cI857 | TCAGCCAAACGTCTCTTCAGGCCACTGACTAGCGATAACTTTCC |
| repressor | CCACAACGGAACAACTCTCATTGCATGGGATCATTGGGTACTGT |
| SEQ ID NO: 31 | GGGTTTAGTGGTTGTAAAAACACCTGACCGCTATCCCTGATCAG |
| | TTTCTTGAAGGTAAACTCATCACCCCCAAGTCTGGCTATGCAGA |
| | AATCACCTGGCTCAACAGCCTGCTCAGGGTCAACGAGAATTAA |
| | CATTCCGTCAGGAAAGCTTGGCTTGGAGCCTGTTGGTGCGGTCA |
| | TGGAATTACCTTCAACCTCAAGCCAGAATGCAGAATCACTGGCT |
| | TTTTTGGTTGTGCTTACCCATCTCTCCGCATCACCTTTGGTAAAG |
| | GTTCTAAGCTTAGGTGAGAACATCCCTGCCTGAACATGAGAAA |
| | AAACAGGGTACTCATACTCACTTCTAAGTGACGGCTGCATACTA |
| | ACCGCTTCATACATCTCGTAGATTTCTCTGGCGATTGAAGGGCT |
| | AAATTCTTCAACGCTAACTTTGAGAATTTTTGTAAGCAATGCGG |
| | CGTTATAAGCATTTAATGCATTGATGCCATTAAATAAAGCACCA |
| | ACGCCTGACTGCCCCATCCCCATCTTGTCTGCGACAGATTCCTG |
| | GGATAAGCCAAGTTCATTTTTCTTTTTTTCATAAATTGCTTTAAG |
| | GCGACGTGCGTCCTCAAGCTGCTCTTGTGTTAATGGTTTCTTTTT |
| | TGTGCTCAT |
| RBS and leader | CTCTAGAAATAATTTTGTTTAACTTTAAGAAGGAGATATACAT |
| region | |
| SEQ ID NO: 32 | |
| mutant cI857 | MSTKKKPLTQEQLEDARRLKAIYEKKKNELGLSQESVADKMGMG |
| repressor | QSGVGALFNGINALNAYNAALLTKILKVSVEEFSPSIAREIYEMYE |
| polypeptide | AVSMQPSLRSEYEYPVFSHVQAGMFSPKLRTFTKGDAERWVSTTK |
| sequence | KASDSAFWLEVEGNSMTAPTGSKPSFPDGMLILVDPEQAVEPGDF |
| SEQ ID NO: 33 | CIARLGGDEFTFKKLIRDSGQVFLQPLNPQYPMIPCNESCSVVGKVI |
| | ASQWPEETFG |
| TetR-Tet | Ttaagacccactttcacatttaagttgtattctaatccgcatatgatcaattcaaggccgaataagaagg |
| promoter | ctggctctgcaccttggtgatcaaataattcgatagcttgtcgtaataatggcggcatactatcagtagta |
| construct | ggtgtttccctttcttctttagcgacttgatgctcttgatcttccaatacgcaacctaaagtaaaatgcccca |
| SEQ ID NO: 34 | cagcgctgagtgcatataatgcattctctagtgaaaaaccttgttggcataaaaaggctaattgattttcg |
| | agagtttcatactgttatctgtaggccgtgtacctaaatgtacttttgctccatcgcgatgacttagtaaag |
| | cacatctaaaacttttagcgttattacgtaaaaaatcttgccagctttccccttctaaagggcaaaagtga |
| | gtatggtgcctatctaacatctcaatggctaaggcgtcgagcaaagcccgcttattttttacatgccaata |
| | caatgtaggctgctctacacctagcttctgggcgagtttacgggttgttaaaccttcgattccgacctcatt |
| | aagcagctctaatgcgctgttaatcactttacttttatctaatctagacatcattaattcctaatttttgttgaca |
| | ctctatcattgatagagttattttaccactccctatcagtgatagagaaaagtgaactctagaaataatt |
| | ttgtttaactttaagaaggagatatacat |
| PssB promoter | tcacctttcccggattaaacgcttttttgcccggtggcatggtgctaccggcgatcacaaacggttaattatg |
| SEQ ID NO: 35 | acacaaattgacctgaatgaatatacagtattggaatgcattacccggagtgttgtgtaacaatgtctggcca |
| | ggtttgtttcccggaaccgaggtcacaacatagtaaaagcgctattggtaatggtacaatcgcgcgtttaca |
| | cttattc |

### Constitutive Promoters

In some embodiments, the gene encoding the payload is present on a plasmid and operably linked to a constitutive promoter. In some embodiments, the gene encoding the payload is present on a chromosome and operably linked to a promoter that is induced by change in temperature from a non-permissive temperature to a permissive temperature. In some embodiments, the gene encoding the payload is present on a chromosome and operably linked to a constitutive promoter.

In some embodiments, the constitutive promoter is active under in vivo conditions, e.g., the gut and/or conditions of the tumor microenvironment, as described herein. In some embodiments, the promoters are active under in vitro conditions, e.g., various cell culture and/or cell manufacturing conditions, as described herein. In some embodiments, the constitutive promoter is active under in vivo conditions, e.g., the gut and/or conditions of the tumor microenvironment, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions). In some embodiments, the constitutive promoter is active in exogenous environmental conditions specific to the gut of a mammal and/or specific to conditions of the tumor microenvironment. In some embodiments, the constitutive promoter is active in exogenous environmental conditions specific to the small intestine of a mammal. In some embodiments, the constitutive promoter is active in low-oxygen or anaerobic conditions such as the environment of the mammalian gut and/or conditions of the tumor microenvironment. In some embodiments, the constitutive promoter is active in the presence of molecules or metabolites that are specific to the gut of a mammal and/or conditions of the tumor microenvironment. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art and are described in International Patent Application PCT/US2017/013072, filed Jan. 11, 2017, published as WO2017/123675, the contents of which is herein incorporated by reference in its entirety.

### Ribosome Binding Sites

In some embodiments, ribosome binding sites are added, switched out or replaced. By testing a few ribosome binding sites, expression levels can be fine-tuned to the desired level. Non-limiting examples of RBS are listed at Registry of standard biological parts and are described in are described in International Patent Application PCT/US2017/013072, filed Jan. 11, 2017, published as WO2017/123675, the contents of which is herein incorporated by reference in its entirety.

### Induction of Payloads During Strain Culture

Induction of payloads during culture is described in International Patent Application PCT/US2017/013072, filed Jan. 11, 2017, published as WO2017/123675, International Patent Applications PCT/US2016/032562, filed—May 13, 2016, published as WO2016183531, and PCT/US2016/062369, filed Nov. 16, 2016 and published as WO2017087580, the contents of each of which are herein incorporated by reference in their entireties.

In some embodiments, it is desirable to pre-induce payload or protein of interest expression and/or payload activity prior to administration. Such payload or protein of interest may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the genetically engineered bacteria of the invention express one or more protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more protein(s) of interest and are directly or indirectly induced, while the strains is grown up for in vivo administration. Without wishing to be bound by theory, pre-induction may boost in vivo activity, e.g., in the gut or a tumor. If the bacterial residence time in a particular gut compartment is relatively short, the bacteria may pass through the small intestine without reaching full in vivo induction capacity. In contrast, if a strain is pre-induced and preloaded, the strains are already fully active, allowing for greater activity more quickly as the bacteria reach the intestine. Ergo, no transit time is “wasted”, in which the strain is not optimally active. As the bacteria continue to move through the intestine, in vivo induction occurs under environmental conditions of the gut (e.g., low oxygen, or in the presence of gut metabolites). Similarly, if a tumor targeting or other bacterium is pre-induced and preloaded, this may allow for greater activity more quickly as the bacteria reach the gut or the tumor, either through systemic administration or intratumor injection, as described herein. Once in the gut or the tumor, in vivo induction occurs, e.g., under conditions of the tumor microenvironment.

In one embodiment, expression of one or more payload(s), is induced during cell growth, cell expansion, fermentation, recovery, purification, formulation, and/or manufacture. In one embodiment, expression of several different proteins of interest is induced during cell growth, cell expansion, fermentation, recovery, purification, formulation, and/or manufacture.

In some embodiments, the strains are administered without any pre-induction protocols during strain growth prior to in vivo administration.

### Anaerobic Induction

In some embodiments, cells are induced under anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then switched to anaerobic or low oxygen conditions for approximately 3 to 5 hours. In some embodiments, strains are induced under anaerobic or low oxygen conditions, e.g. to induce FNR promoter activity and drive expression of one or more payload(s) and/or transporters under the control of one or more FNR promoters.

In one embodiment, expression of one or more payload(s), is under the control of one or more FNR promoter(s) and is induced during cell growth, cell expansion, fermentation, recovery, purification, formulation, and/or manufacture under anaerobic or low oxygen conditions. In one embodiment, expression of several different proteins of interest is under the control of one or more FNR promoter(s) and is induced during cell growth, cell expansion, fermentation, recovery, purification, formulation, and/or manufacture under anaerobic or low oxygen conditions.

Without wishing to be bound by theory, strains that comprise one or more payload(s) under the control of an FNR promoter, may allow expression of payload(s) from these promoters in vitro, under anaerobic or low oxygen culture conditions, and in vivo, under the low oxygen conditions found in the gut and/or conditions of the tumor microenvironment.

In some embodiments, promoters linked to the payload of interest may be inducible by arabinose, cumate, and salicylate, IPTG, rhamnose, tetracycline, and/or other chemical and/or nutritional inducers can be induced under anaerobic or low oxygen conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) and/or under the control of one or more FNR promoter(s), and one or more payload gene sequence(s) under the control of a one or more constitutive promoter(s) described herein.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to a permissive temperature, for approximately 3 to 5 hours.

In some embodiments, promoters inducible by arabinose, cumate, and salicylate, IPTG, rhamnose, tetracycline, and/or other chemical and/or nutritional inducers described herein or known in the art can be induced under aerobic conditions in the presence of the chemical and/or nutritional inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut and/or conditions of the tumor microenvironment. In some embodiments, these strains do not further comprise FNR inducible gene sequence(s) for in vivo activation in the gut and/or conditions of the tumor microenvironment.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) are driven by an anaerobic and/or low oxygen promoter, e.g., a FNR promoter. In such instances, cells are for example grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

Without wishing to be bound by theory, strains that comprise one or more payload(s) under the control of an FNR promoter, may allow expression of payload(s) from these promoters in vitro, under microaerobic culture conditions, and in vivo, under the low oxygen conditions found in the gut and/or conditions of the tumor microenvironment.

In some embodiments, promoters inducible by arabinose, cumate, and salicylate, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s), and one or more payload gene sequence(s) under the control of a one or more constitutive promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

In some embodiments, it is desirable to pre-induce payload or protein of interest expression and/or payload activity prior to administration. Such payload or protein of interest may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or

Generation of Bacterial Strains with Enhanced Ability to Transport Biomolecules

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid—at growth-limiting concentrations—will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

In the previous examples, a metabolite innate to the microbe was made essential via mutational auxotrophy and selection was applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate. Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

### Metabolite Transport

In some embodiments, the genetically engineered bacteria further comprise a gene encoding a transporter. Transporters may be expressed or modified in the genetically engineered bacteria of the invention in order to enhance phenylalanine transport into the cell. Non-limiting examples of such transporters are described in pending International Patent Application PCT/US2016/032565, the contents of which is herein incorporated by reference in its entirety.

Such transporters are membrane transport protein that is capable of transporting metabolites into bacterial cells. In some embodiments, the gene encoding the transporter in the genetically modified bacteria of the invention is not modified. In some embodiments, the genetically engineered bacteria of the invention comprise multiple copies of the transporter gene. In some embodiments, the genetically engineered bacteria of the invention comprise multiple copies of a non-native transporter gene. In some embodiments, the genetically engineered bacteria of the invention comprise a transporter gene that is controlled by its native promoter, an inducible promoter, a promoter that is stronger than the native promoter, e.g., the GlnRS promoter or the P(Bla) promoter, or a constitutive promoter. In some embodiments, the promoter is induced under conditions during manufacture or other in vitro conditions. In some embodiments, expression of the transporter gene is controlled by a different promoter than the promoter that controls expression of the gene encoding the one or more effector molecule. In some embodiments, expression of the transporter gene is controlled by the same promoter that controls expression of the one or more effector molecules. In some embodiments, the transporter gene and the one or more effector molecules are divergently transcribed from a promoter region.

In some embodiments, the native transporter gene is mutagenized, mutants exhibiting increased transport are selected, and the mutagenized transporter gene is isolated and inserted into the genetically engineered bacteria (see, e.g., Pi et al., 1996; Pi et al., 1998).

In some embodiments, the genetically engineered bacteria of the invention produce PAL under exogenous environmental conditions, such as the low-oxygen environment of the mammalian gut, to reduce blood phenylalanine by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions. In some embodiments, the genetically engineered bacteria of the invention produce PAL under exogenous environmental conditions, such as the low-oxygen environment of the mammalian gut, and increase hippuric acid in the urine by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions. Certain unmodified bacteria will not have appreciable levels of phenylalanine processing to hippurate. In embodiments using genetically modified forms of these bacteria, PAL-mediated processing of phenylalanine will be appreciable under exogenous environmental conditions.

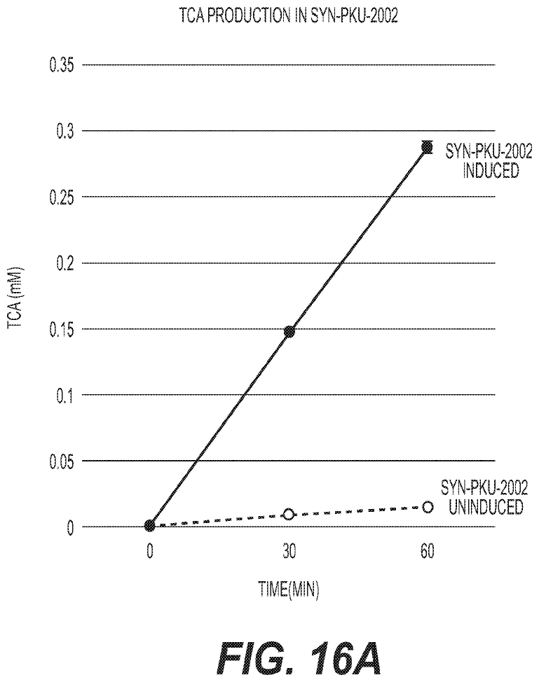

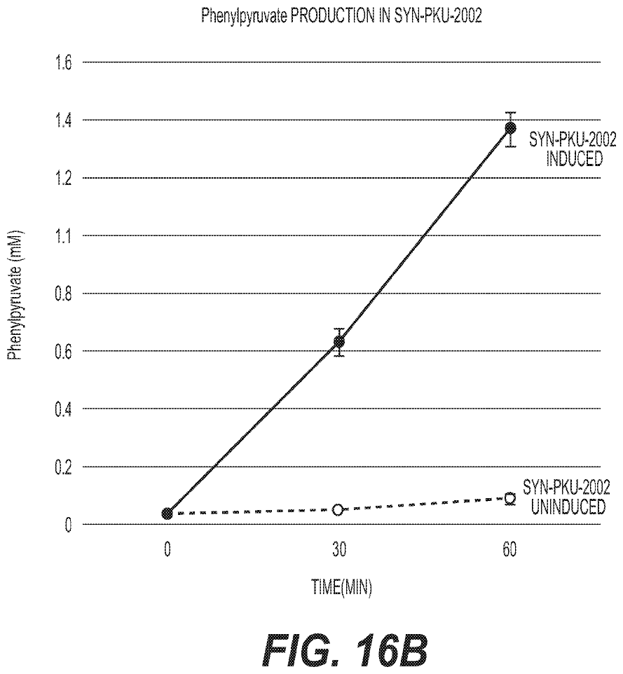

In some embodiments, the genetically engineered bacteria of the invention produce PAL under exogenous environmental conditions, such as under bacterial culture conditions in vitro, and increase transcinnamic acid in the media by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions. Phenylalanine may be measured by methods known in the art, e.g., blood sampling and mass spectrometry. In some embodiments, cinnamate is measured by methods known in the art to assess PAL activity. Cinnamate production is directly correlated with phenylalanine degradation, and in some embodiments, that cinnamate may be used as an alternative biomarker for strain activity (FIG. 16B). Cinnamate can be further degraded to hippuric acid by liver enzymes; both can be measured as described in Example 24-26. As shown herein, TCA is rapidly converted to hippuric acid in vivo, and hippuric acid subsequently accumulates in the urine. Therefore, hippurate, in blood and in particular in the urine, may be an even better biomarker for phenylalanine degradation in vivo. In some embodiments, PAL expression is measured by methods known in the art, e.g., measurement of blood phenylalanine levels. Hippuric acid may be measured according to methods described herein in the Examples, and methods known in the art.

In some embodiments, the genetically engineered bacteria of the invention produce LAAD, to reduce blood phenylalanine by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions. Certain unmodified bacteria will not have appreciable levels of phenylalanine processing. In embodiments using genetically modified forms of these bacteria, LAAD-mediated processing of phenylalanine will be appreciable under exogenous environmental conditions. Phenylalanine may be measured by methods known in the art, e.g., blood sampling and mass spectrometry. Pyruvic acid and phenylpyruvate, the LAAD generated degradation products can be measured using mass spectrometry as described in Examples 24-26, and can be used as an additional readout of LAAD activity.

In some embodiments, the genetically engineered bacteria of the invention produce more than one PME, e.g., PAL, PAH, and/or LAAD, under exogenous environmental conditions, such as in vivo or under bacterial culture conditions in vitro, and reduce blood phenylalanine and/or increase transcinnamic acid in the media by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions. In any of these embodiments, the bacteria may further comprise gene sequence(s) encoding one or more Phe transporter polypeptides.

In some embodiments, one or more PME(s), e.g., PAL, LAAD, and/or PAH, are expressed on a low-copy plasmid.

In some embodiments, the gene(s) encoding the one or more transporter(s) is located on a plasmid or in the chromosome and expression may be regulated by any of the promoters disclosed herein.

In other embodiments, the genetically engineered bacteria encode one or more transporter(s) which are directly or indirectly induced in vivo administration, e.g., are expressed under the control of an inducible promoter that is responsive conditions or to specific molecules or metabolites in the exogenous in vivo environment, e.g., the gut. In some embodiments, the promoter is induced by gut specific molecules, or low oxygen conditions. In some embodiments, the bacterial strains are administered in combination with a chemical and/or nutritional inducer.

In some embodiments, one or more PME(s), e.g., PAL, LAAD, and/or PAH, are expressed on a high-copy plasmid. In some embodiments, the high-copy plasmid may be useful for increasing the PME, e.g., PAL, LAAD, and/or PAH, expression, thereby increasing the metabolism of phenylalanine and reducing hyperphenylalaninemia. In some embodiments, a genetically engineered bacterium comprising a the PME, e.g., PAL, LAAD, and/or PAH, expressed on a high-copy plasmid does not increase phenylalanine metabolism or decrease phenylalanine levels as compared to a genetically engineered bacterium comprising the same PME, e.g., PAL, LAAD, and/or PAH, expressed on a low-copy plasmid in the absence of heterologous pheP and additional copies of a native pheP. Genetically engineered bacteria comprising the same the PME gene(s), e.g., PAL, LAAD, and/or PAH gene(s) on high and low copy plasmids were generated. For example, either PAL1 or PAL3 on a high-copy plasmid and a low-copy plasmid were generated, and each metabolized and reduced phenylalanine to similar levels (FIG. 15). Thus, in some embodiments, the rate-limiting step of phenylalanine metabolism is phenylalanine availability (see, e.g., FIG. 16). In these embodiments, it may be advantageous to increase phenylalanine transport into the cell, thereby enhancing phenylalanine metabolism. In conjunction with pheP, even low-copy PAL plasmids are capable of almost completely eliminating Phe from a test sample (see, e.g., FIG. 16A). Furthermore, in some embodiments, that incorporate pheP, there may be additional advantages to using a low-copy PAL-expressing plasmid in conjunction in order to enhance the stability of PAL expression while maintaining high phenylalanine metabolism, and to reduce negative selection pressure on the transformed bacterium. In alternate embodiments, the phenylalanine transporter is used in conjunction with the high-copy plasmid.

In some embodiments, a transporter may not increase phenylalanine degradation. For example, Proteus mirabilis LAAD is localized to the plasma membrane, with the enzymatic catalysis occurring in the periplasm. Phenylalanine can readily traverse the outer membrane without the need of a transporter. Therefore, in embodiments, in which the genetically engineered bacteria express LAAD, a transporter may not be needed or improve phenylalanine metabolism.

In some embodiments, the PME(s), e.g., PAL, LAAD, and/or PAH, gene(s) are expressed on a chromosome. In some embodiments, expression from the chromosome may be useful for increasing stability of expression of the PME. In some embodiments, the PME gene, e.g., PAL, LAAD, and/or PAH gene(s), is integrated into the bacterial chromosome at one or more integration sites in the genetically engineered bacteria. In some embodiments, the PME gene, e.g., PAL, LAAD, and/or PAH gene(s) is inserted into the bacterial genome at one or more of the following insertion sites in E. coli Nissle: malE/K, insB/I, araC/BAD, lacZ, agal/rsml, thyA, and malP/T. Any suitable insertion site may be used (see, e.g., FIG. 66 of WO2017087580, the contents of which are herein incorporated by reference in their entirety). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. In some embodiments, more than one copy, e.g., two, three, four, five, six, seven, eight, nine, ten or more copies of the PME gene, e.g., PAL, PAH, and/or LAAD is integrated into the bacterial chromosome at one or more integration sites in the genetically engineered bacteria. The more than one copy of a PME gene may be more than one copy of the same PME gene or more than one copy of different PME genes.

Exemplary constructs are shown in Tables 4-13 below. Table 4 shows the sequence of an exemplary construct comprising a gene encoding PheP and an FNR promoter sequence for chromosomal insertion (SEQ ID NO: 21B), with the pheP sequence underlined and the FNR promoter sequence bolded. Table 5 shows the sequence of an exemplary construct comprising a gene encoding PAL1 and an FNR promoter sequence on a high-copy plasmid (SEQ ID NO: 22B), with the PAL1 sequence underlined and the FNR promoter sequence bolded. Table 6 shows the sequence of an exemplary construct comprising a gene encoding PAL3 and an FNR promoter sequence on a high-copy plasmid (SEQ ID NO: 23B), with the PAL3 sequence underlined and the FNR promoter sequence bolded. Table 7 shows the sequence of an exemplary construct comprising a gene encoding PAL1 and a Tet promoter sequence on a high-copy plasmid (SEQ ID NO: 24B), with the PAL1 sequence underlined and the Tet promoter sequence bolded. Table 8 shows the sequence of an exemplary construct comprising a gene encoding PAL3 and a Tet promoter sequence on a high-copy plasmid (SEQ ID NO: 25B), with the PAL3 sequence underlined and the Tet promoter sequence bolded. Table 9 shows the sequence of an exemplary construct comprising a gene encoding PAL1 and an FNR promoter sequence on a low-copy plasmid (SEQ ID NO: 26B), with the PAL1 sequence underlined and the FNR promoter sequence bolded. Table 10 shows the sequence of an exemplary construct comprising a gene encoding PAL3 and an FNR promoter sequence on a low-copy plasmid (SEQ ID NO: 27B), with the PAL3 sequence underlined and the FNR promoter sequence bolded. Table 11 shows the sequence of an exemplary construct comprising a gene encoding PAL1 and a Tet promoter sequence on a low-copy plasmid (SEQ ID NO: 28B), with the PAL1 sequence underlined and the Tet promoter sequence bolded. Table 12 shows the sequence of an exemplary construct comprising a gene encoding PAL3 and a Tet promoter sequence on a low-copy plasmid (SEQ ID NO: 29B), with the PAL3 sequence underlined and the Tet promoter sequence bolded. Table 13 shows the sequence of an exemplary construct comprising a gene encoding PheP, a gene coding TetR, and a Tet promoter sequence for chromosomal insertion (SEQ ID NO: 30B), with the pheP sequence underlined, the TetR sequence [symbol] , and the FNR promoter sequence bolded.

TABLE 4
Nucleotide sequences of FNR promoter-PheP
construct (SEQ ID NO: 21B)

CTCTTGATCGTTATCAATTCCCACGCTGTTTCAGAGCGTTACCTTGCCCTT
AAACATTAGCAATGTCGATTTATCAGAGGGCCGACAGGCTCCCACAGGAGA
AAACCGATGAAAAACGCGTCAACCGTATCGGAAGATACTGCGTCGAATCAA
GAGCCGACGCTTCATCGCGGATTACATAACCGTCATATTCAACTGATTGCG
TTGGGTGGCGCAATTGGTACTGGTCTGTTTCTTGGCATTGGCCCGGCGATT
CAGATGGCGGGTCCGGCTGTATTGCTGGGCTACGGCGTCGCCGGGATCATC
GCTTTCCTGATTATGCGCCAGCTTGGCGAAATGGTGGTTGAGGAGCCGGTA
TCCGGTTCATTTGCCCACTTTGCCTATAAATACTGGGGACCGTTTGCGGGC
TTCCTCTCTGGCTGGAACTACTGGGTAATGTTCGTGCTGGTGGGAATGGCA
GAGCTGACCGCTGCGGGCATCTATATGCAGTACTGGTTCCCGGATGTTCCA
ACGTGGATTTGGGCTGCCGCCTTCTTTATTATCATCAACGCCGTTAACCTG
GTGAACGTGCGCTTATATGGCGAAACCGAGTTCTGGTTTGCGTTGATTAAA
GTGCTGGCAATCATCGGTATGATCGGCTTTGGCCTGTGGCTGCTGTTTTCT
GGTCACGGCGGCGAGAAAGCCAGTATCGACAACCTCTGGCGCTACGGTGGT
TTCTTCGCCACCGGCTGGAATGGGCTGATTTTGTCGCTGGCGGTAATTATG
TTCTCCTTCGGCGGTCTGGAGCTGATTGGGATTACTGCCGCTGAAGCGCGC
GATCCGGAAAAAAGCATTCCAAAAGCGGTAAATCAGGTGGTGTATCGCATC
CTGCTGTTTTACATCGGTTCACTGGTGGTTTTACTGGCGCTCTATCCGTGG
GTGGAAGTGAAATCCAACAGTAGCCCGTTTGTGATGATTTTCCATAATCTC
GACAGCAACGTGGTAGCTTCTGCGCTGAACTTCGTCATTCTGGTAGCATCG
CTGTCAGTGTATAACAGCGGGGTTTACTCTAACAGCCGCATGCTGTTTGGC
CTTTCTGTGCAGGGTAATGCGCCGAAGTTTTTGACTCGCGTCAGCCGTCGC
GGTGTGCCGATTAACTCGCTGATGCTTTCCGGAGCGATCACTTCGCTGGTG
GTGTTAATCAACTATCTGCTGCCGCAAAAAGCGTTTGGTCTGCTGATGGCG
CTGGTGGTAGCAACGCTGCTGTTGAACTGGATTATGATCTGTCTGGCGCAT
CTGCGTTTTCGTGCAGCGATGCGACGTCAGGGGCGTGAAACACAGTTTAAG
GCGCTGCTCTATCCGTTCGGCAACTATCTCTGCATTGCCTTCCTCGGCATG
ATTTTGCTGCTGATGTGCACGATGGATGATATGCGCTTGTCAGCGATCCTG
CTGCCGGTGTGGATTGTATTCCTGTTTATGGCATTTAAAACGCTGCGTCGG
AAATAA

TABLE 5
Nucleotide sequences of FNR promoter-PAL1 con-
struct, high-copy (SEQ ID NO: 22B)

CTCTTGATCGTTATCAATTCCCACGCTGTTTCAGAGCGTTACCTTGCCCTT
AAACATTAGCAATGTCGATTTATCAGAGGGCCGACAGGCTCCCACAGGAGA
AAACCGATGAAAACACTATCACAGGCCCAATCTAAAACTTCTTCACAGCAA
TTCAGCTTTACCGGGAACTCGTCTGCGAATGTAATTATCGGCAATCAAAAG
CTGACCATTAATGATGTAGCTCGCGTTGCCCGGAATGGCACTTTGGTGTCA
CTGACGAACAATACCGACATTCTGCAAGGTATTCAAGCTAGCTGCGATTAT
ATCAATAACGCCGTTGAATCTGGCGAGCCAATCTACGGGGTAACAAGCGGT
TTTGGTGGGATGGCGAACGTTGCCATTAGCCGTGAACAGGCGAGCGAACTT
CAGACCAACCTCGTTTGGTTCCTAAAGACAGGAGCTGGTAATAAGTTACCT
CTGGCTGACGTAAGAGCCGCGATGCTGCTTCGCGCTAATAGTCACATGCGC
GGCGCCAGTGGTATCCGTCTTGAGCTTATCAAGAGGATGGAAATCTTCCTC
AACGCGGGTGTCACACCATATGTTTATGAGTTTGGTAGTATCGGAGCCAGT
GGTGATCTTGTTCCCCTGAGTTATATTACGGGTTCATTGATTGGTTTAGAC
CCGTCCTTTAAAGTGGATTTTAACGGGAAAGAAATGGACGCCCCGACCGCT
TTACGACAGCTTAATCTGAGCCCACTTACTTTGCTCCCTAAAGAAGGTCTT
GCCATGATGAATGGCACCTCTGTGATGACTGGAATTGCCGCGAATTGTGTG
TATGACACGCAGATCCTAACGGCCATTGCCATGGGTGTTCACGCGTTGGAC
ATTCAAGCCCTGAATGGTACAAACCAGTCGTTTCATCCGTTTATCCATAAT
TCAAAACCCCATCCGGGACAGCTTTGGGCTGCTGATCAGATGATCTCACTC
CTGGCCAATAGTCAACTGGTTCGGGACGAGCTCGACGGCAAACATGATTAT
CGCGATCATGAGCTCATCCAGGACCGGTATTCACTTCGTTGTCTCCCACAA
TACCTGGGGCCTATCGTTGATGGTATATCTCAAATTGCGAAGCAAATTGAA
ATTGAGATCAATAGCGTAACCGACAACCCGCTTATCGATGTTGATAATCAG
GCCTCTTATCACGGTGGCAATTTTCTGGGCCAGTATGTTGGTATGGGGATG
GATCACCTGCGGTACTATATTGGGCTTCTGGCTAAACATCTTGATGTGCAG
ATTGCCTTATTAGCTTCACCAGAATTTTCAAATGGACTGCCGCCATCATTG
CTCGGTAACAGAGAAAGGAAAGTAAATATGGGCCTTAAGGGCCTTCAGATA
TGTGGTAACTCAATCATGCCCCTCCTGACCTTTTATGGGAACTCAATTGCT
GATCGTTTTCCGACACATGCTGAACAGTTTAACCAAAACATTAACTCACAG
GGCTATACATCCGCGACGTTAGCGCGTCGGTCCGTGGATATCTTCCAGAAT
TATGTTGCTATCGCTCTGATGTTCGGCGTACAGGCCGTTGATTTGCGCACT
TATAAAAAAACCGGTCACTACGATGCTCGGGCTTGCCTGTCGCCTGCCACC
GAGCGGCTTTATAGCGCCGTACGTCATGTTGTGGGTCAGAAACCGACGTCG
GACCGCCCCTATATTTGGAATGATAATGAACAAGGGCTGGATGAACACATC
GCCCGGATATCTGCCGATATTGCCGCCGGAGGTGTCATCGTCCAGGCGGTA
CAAGACATACTTCCTTGCCTGCATTAAGCTTGGCGTAATCATGGTCATAGC
TGTTTCCTGTGTGAAATTGTTATCCGCTCACAATTCCACACAACATACGAG
CCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAA
AGGCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACC
CGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTC
AGTGGAACGAAAACTCACGTTAAGGGATTTTGGTCATGAGATTATCAAAAA
GGATCTTCACCTAGATCCTTTTAAATTAAAAATGAAGTTTTAAATCAATCT
AAAGTATATATGAGTAAACTTGGTCTGACAGTTACCAATGCTTAATCAGTG
AGGCACCTATCTCAGCGATCTGTCTATTTCGTTCATCCATAGTTGCCTGAC
TCCCCGTCGTGTAGATAACTACGATACGGGAGGGCTTACCATCTGGCCCCA
GTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAA
GATGCTTTTCTGTGACTGGTGAGTACTCAACCAAGTCATTCTGAGAATAGT
GTATGCGGCGACCGAGTTGCTCTTGCCCGGCGTCAATACGGGATAATACCG
CGCCACATAGCAGAACTTTAAAAGTGCTCATCATTGGAAAACGTTCTTCGG
GGCGAAAACTCTCAAGGATCTTACCGCTGTTGAGATCCAGTTCGATGTAAC
CCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGG
CGACACGGAAATGTTGAATACTCATACTCTTCCTTTTTCAATATTATTGAA
GCATTTATCAGGGTTATTGTCTCATGAGCGGATACATATTTGAATGTATTT
AGAAAAATAAACAAATAGGGGTTCCGCGCACATTTCCCCGAAAAGTGCCAC
CTGACGTCTAAGAAACCATTATTATCATGACATTAACCTATAAAAATAGGC
GTATCACGAGGCCCTTTCGTCTCGCGCGTTTCGGTGATGACGGTGAAAACC
TCTGACACATGCAGCTCCCGGAGACGGTCACAGCTTGTCTGTAAGCGGATG
CCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACC
ATATGCGGTGTGAAATACCGCACAGATGCGTAAGGAGAAAATACCGCATCA
GGCGCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGC
GATTAAGTTGGGTAACGCCAGGGTTTTCCCAGTCACGACGTT

TABLE 6
Nucleotide sequences of FNR promoter-PAL3 con-
struct, high-copy (SEQ ID NO: 23B)

CTCTTGATCGTTATCAATTCCCACGCTGTTTCAGAGCGTTACCTTGCCCTT
AAACATTAGCAATGTCGATTTATCAGAGGGCCGACAGGCTCCCACAGGAGA
AAACCGATGAAAGCTAAAGATGTTCAGCCAACCATTATTATTAATAAAAAT
GGCCTTATCTCTTTGGAAGATATCTATGACATTGCGATAAAACAAAAAAAA
GTAGAAATATCAACGGAGATCACTGAACTTTTGACGCATGGTCGTGAAAAA
TTAGAGGAAAAATTAAATTCAGGAGAGGTTATATATGGAATCAATACAGGA
TTTGGAGGGAATGCCAATTTAGTTGTGCCATTTGAGAAAATCGCAGAGCAT
CAGCAAAATCTGTTAACTTTTCTTTCTGCTGGTACTGGGGACTATATGTCC
AAACCTTGTATTAAAGCGTCACAATTTACTATGTTACTTTCTGTTTGCAAA
GGTTGGTCTGCAACCAGACCAATTGTCGCTCAAGCAATTGTTGATCATATT
AATCATGACATTGTTCCTCTGGTTCCTCGCTATGGCTCAGTGGGTGCAAGC
GGTGATTTAATTCCTTTATCTTATATTGCACGAGCATTATGTGGTATCGGC
AAAGTTTATTATATGGGCGCAGAAATTGACGCTGCTGAAGCAATTAAACGT
GCAGGGTTGACACCATTATCGTTAAAAGCCAAAGAAGGTCTTGCTCTGATT
AACGGCACCCGGGTAATGTCAGGAATCAGTGCAATCACCGTCATTAAACTG
GAAAAACTATTTAAAGCCTCAATTTCTGCGATTGCCCTTGCTGTTGAAGCA
TTACTTGCATCTCATGAACATTATGATGCCCGGATTCAACAAGTAAAAAAT
CATCCTGGTCAAAACGCGGTGGCAAGTGCATTGCGTAATTTATTGGCAGGT
TCAACGCAGGTTAATCTATTATCTGGGGTTAAAGAACAAGCCAATAAAGCT
TGTCGTCATCAAGAAATTACCCAACTAAATGATACCTTACAGGAAGTTTAT
TCAATTCGCTGTGCACCACAAGTATTAGGTATAGTGCCAGAATCTTTAGCT
ACCGCTCGGAAAATATTGGAACGGGAAGTTATCTCAGCTAATGATAATCCA
TTGATAGATCCAGAAAATGGCGATGTTCTACACGGTGGAAATTTTATGGGG
CAATATGTCGCCCGAACAATGGATGCATTAAAACTGGATATTGCTTTAATT
GCCAATCATCTTCACGCCATTGTGGCTCTTATGATGGATAACCGTTTCTCT
CGTGGATTACCTAATTCACTGAGTCCGACACCCGGCATGTATCAAGGTTTT
AAAGGCGTCCAACTTTCTCAAACCGCTTTAGTTGCTGCAATTCGCCATGAT
TGTGCTGCATCAGGTATTCATACCCTCGCCACAGAACAATACAATCAAGAT
ATTGTCAGTTTAGGTCTGCATGCCGCTCAAGATGTTTTAGAGATGGAGCAG
AAATTACGCAATATTGTTTCAATGACAATTCTGGTAGTTTGTCAGGCCATT
CATCTTCGCGGCAATATTAGTGAAATTGCGCCTGAAACTGCTAAATTTTAC
CATGCAGTACGCGAAATCAGTTCTCCTTTGATCACTGATCGTGCGTTGGAT
GAAGATATAATCCGCATTGCGGATGCAATTATTAATGATCAACTTCCTCTG
CCAGAAATCATGCTGGAAGAATAAGCTTGGCGTAATCATGGTCATAGCTGT
TTCCTGTGTGAAATTGTTATCCGCTCACAATTCCACACAACATACGAGCCG
GAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTG
GGCGCTCTTCCGCTTCCTCGCTCACTGACTCGCTGCGCTCGGTCGTTCGGC
TGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGG
TAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGT
GGAACGAAAACTCACGTTAAGGGATTTTGGTCATGAGATTATCAAAAAGGA
TCTTCACCTAGATCCTTTTAAATTAAAAATGAAGTTTTAAATCAATCTAAA
GTATATATGAGTAAACTTGGTCTGACAGTTACCAATGCTTAATCAGTGAGG
CACCTATCTCAGCGATCTGTCTATTTCGTTCATCCATAGTTGCCTGACTCC
CCGTCGTGTAGATAACTACGATACGGGAGGGCTTACCATCTGGCCCCAGTG
CTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTCA
TGGTTATGGCAGCACTGCATAATTCTCTTACTGTCATGCCATCCGTAAGAT
GCTTTTCTGTGACTGGTGAGTACTCAACCAAGTCATTCTGAGAATAGTGTA
TGCGGCGACCGAGTTGCTCTTGCCCGGCGTCAATACGGGATAATACCGCGC
CACATAGCAGAACTTTAAAAGTGCTCATCATTGGAAAACGTTCTTCGGGGC
GAAAACTCTCAAGGATCTTACCGCTGTTGAGATCCAGTTCGATGTAACCCA
CTCGTGCACCCAACTGATCTTCAGCATCTTTTACTTTCACCAGCGTTTCTG
GGTGAGCAAAAACAGGAAGGCAAAATGCCGCAAAAAAGGGAATAAGGGCGA
CACGGAAATGTTGAATACTCATACTCTTCCTTTTTCAATATTATTGAAGCA
TTTATCAGGGTTATTGTCTCATGAGCGGATACATATTTGAATGTATTTAGA
AAAATAAACAAATAGGGGTTCCGCGCACATTTCCCCGAAAAGTGCCACCTG
ACGTCTAAGAAACCATTATTATCATGACATTAACCTATAAAAATAGGCGTA
TCACGAGGCCCTTTCGTCTCGCGCGTTTCGGTGATGACGGTGAAAACCTCT
GACACATGCAGCTCCCGGAGACGGTCACAGCTTGTCTGTAAGCGGATGCCG
GGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATA
TGCGGTGTGAAATACCGCACAGATGCGTAAGGAGAAAATACCGCATCAGGC
GCCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGAT
TAAGTTGGGTAACGCCAGGGTTTTCCCAGTCACGACGTT

TABLE 7
Nucleotide sequences of Tet promoter-PAL1 con-
struct, high-copy (SEQ ID NO: 24B)

CTCTAGAAATAATTTTGTTTAACTTTAAGAAGGAGATATACATATGAAAAC
ACTATCACAGGCCCAATCTAAAACTTCTTCACAGCAATTCAGCTTTACCGG
GAACTCGTCTGCGAATGTAATTATCGGCAATCAAAAGCTGACCATTAATGA
TGTAGCTCGCGTTGCCCGGAATGGCACTTTGGTGTCACTGACGAACAATAC
CGACATTCTGCAAGGTATTCAAGCTAGCTGCGATTATATCAATAACGCCGT
TGAATCTGGCGAGCCAATCTACGGGGTAACAAGCGGTTTTGGTGGGATGGC
GAACGTTGCCATTAGCCGTGAACAGGCGAGCGAACTTCAGACCAACCTCGT
TTGGTTCCTAAAGACAGGAGCTGGTAATAAGTTACCTCTGGCTGACGTAAG
AGCCGCGATGCTGCTTCGCGCTAATAGTCACATGCGCGGCGCCAGTGGTAT
CCGTCTTGAGCTTATCAAGAGGATGGAAATCTTCCTCAACGCGGGTGTCAC
ACCATATGTTTATGAGTTTGGTAGTATCGGAGCCAGTGGTGATCTTGTTCC
CCTGAGTTATATTACGGGTTCATTGATTGGTTTAGACCCGTCCTTTAAAGT
GGATTTTAACGGGAAAGAAATGGACGCCCCGACCGCTTTACGACAGCTTAA
TCTGAGCCCACTTACTTTGCTCCCTAAAGAAGGTCTTGCCATGATGAATGG
CACCTCTGTGATGACTGGAATTGCCGCGAATTGTGTGTATGACACGCAGAT
CCTAACGGCCATTGCCATGGGTGTTCACGCGTTGGACATTCAAGCCCTGAA
TGGTACAAACCAGTCGTTTCATCCGTTTATCCATAATTCAAAACCCCATCC
GGGACAGCTTTGGGCTGCTGATCAGATGATCTCACTCCTGGCCAATAGTCA
ACTGGTTCGGGACGAGCTCGACGGCAAACATGATTATCGCGATCATGAGCT
CATCCAGGACCGGTATTCACTTCGTTGTCTCCCACAATACCTGGGGCCTAT
CGTTGATGGTATATCTCAAATTGCGAAGCAAATTGAAATTGAGATCAATAG
CGTAACCGACAACCCGCTTATCGATGTTGATAATCAGGCCTCTTATCACGG
TGGCAATTTTCTGGGCCAGTATGTTGGTATGGGGATGGATCACCTGCGGTA
CTATATTGGGCTTCTGGCTAAACATCTTGATGTGCAGATTGCCTTATTAGC
TTCACCAGAATTTTCAAATGGACTGCCGCCATCATTGCTCGGTAACAGAGA
AAGGAAAGTAAATATGGGCCTTAAGGGCCTTCAGATATGTGGTAACTCAAT
CATGCCCCTCCTGACCTTTTATGGGAACTCAATTGCTGATCGTTTTCCGAC
ACATGCTGAACAGTTTAACCAAAACATTAACTCACAGGGCTATACATCCGC
GACGTTAGCGCGTCGGTCCGTGGATATCTTCCAGAATTATGTTGCTATCGC
TCTGATGTTCGGCGTACAGGCCGTTGATTTGCGCACTTATAAAAAAACCGG
TCACTACGATGCTCGGGCTTGCCTGTCGCCTGCCACCGAGCGGCTTTATAG
CGCCGTACGTCATGTTGTGGGTCAGAAACCGACGTCGGACCGCCCCTATAT
TTGGAATGATAATGAACAAGGGCTGGATGAACACATCGCCCGGATATCTGC
CGATATTGCCGCCGGAGGTGTCATCGTCCAGGCGGTACAAGACATACTTCC
TTGCCTGCATTAAGCTTGGCGTAATCATGGTCATAGCTGTTTCCTGTGTGA
AATTGTTATCCGCTCACAATTCCACACAACATACGAGCCGGAAGCATAAAG
TGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAA
TGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGAC
TTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAAC
TCACGTTAAGGGATTTTGGTCATGAGATTATCAAAAAGGATCTTCACCTAG
ATCCTTTTAAATTAAAAATGAAGTTTTAAATCAATCTAAAGTATATATGAG
TAAACTTGGTCTGACAGTTACCAATGCTTAATCAGTGAGGCACCTATCTCA
GCGATCTGTCTATTTCGTTCATCCATAGTTGCCTGACTCCCCGTCGTGTAG
ATAACTACGATACGGGAGGGCTTACCATCTGGCCCCAGTGCTGCAATGATA
CCGCGAGACCCACGCTCACCGGCTCCAGATTTATCAGCAATAAACCAGCCA
GCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTT
ACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTG
ACTGGTGAGTACTCAACCAAGTCATTCTGAGAATAGTGTATGCGGCGACCG
AGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCC
AACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGT
TGAATACTCATACTCTTCCTTTTTCAATATTATTGAAGCATTTATCAGGGT
TATTGTCTCATGAGCGGATACATATTTGAATGTATTTAGAAAAATAAACAA
ATAGGGGTTCCGCGCACATTTCCCCGAAAAGTGCCACCTGACGTCTAAGAA
ACCATTATTATCATGACATTAACCTATAAAAATAGGCGTATCACGAGGCCC
TTTCGTCTCGCGCGTTTCGGTGATGACGGTGAAAACCTCTGACACATGCAG
CTCCCGGAGACGGTCACAGCTTGTCTGTAAGCGGATGCCGGGAGCAGACAA
GCCCGTCAGGGCGCGTCAGCGGGTGTTGGCGGGTGTCGGGGCTGGCTTAAC
TATGCGGCATCAGAGCAGATTGTACTGAGAGTGCACCATATGCGGTGTGAA
ATACCGCACAGATGCGTAAGGAGAAAATACCGCATCAGGCGCCATTCGCCA
TTCAGGCTGCGCAACTGTTGGGAAGGGCGATCGGTGCGGGCCTCTTCGCTA
TTACGCCAGCTGGCGAAAGGGGGATGTGCTGCAAGGCGATTAAGTTGGGTA
ACGCCAGGGTTTTCCCAGTCACGACGTTGTAAAACGACGGCCAGTGAATTC
GTTAAGACCCACTTTCACATTTAAGTTGTTTTTCTAATCCGCATATGATCA
ATTCAAGGCCGAATAAGAAGGCTGGCTCTGCACCTTGGTGATCAAATAATT
CGATAGCTTGTCGTAATAATGGCGGCATACTATCAGTAGTAGGTGTTTCCC
TTTCTTCTTTAGCGACTTGATGCTCTTGATCTTCCAATACGCAACCTAAAG
TAAAATGCCCCACAGCGCTGAGTGCATATAATGCATTCTCTAGTGAAAAAC
CTTGTTGGCATAAAAAGGCTAATTGATTTTCGAGAGTTTCATACTGTTTTT
CTGTAGGCCGTGTACCTAAATGTACTTTTGCTCCATCGCGATGACTTAGTA
AAGCACATCTAAAACTTTTAGCGTTATTACGTAAAAAATCTTGCCAGCTTT
CCCCTTCTAAAGGGCAAAAGTGAGTATGGTGCCTATCTAACATCTCAATGG
CTAAGGCGTCGAGCAAAGCCCGCTTATTTTTTACATGCCAATACAATGTAG
GCTGCTCTACACCTAGCTTCTGGGCGAGTTTACGGGTTGTTAAACCTTCGA
TTCCGACCTCATTAAGCAGCTCTAATGCGCTGTTAATCACTTTACTTTTAT
CTAATCTAGACATCATTAATTCCTAATTTTTGTTGACACTCTATCATTGAT
AGAGTTATTTTACCACTCCCTATCAGTGATAGAGAAAAGTGAA

TABLE 8
Nucleotide sequences of Tet promoter-PAL3, high-
copy construct (SEQ ID NO: 25B)

CTCTAGAAATAATTTTGTTTAACTTTAAGAAGGAGATATACATATGAAAGC
TAAAGATGTTCAGCCAACCATTATTATTAATAAAAATGGCCTTATCTCTTT
GGAAGATATCTATGACATTGCGATAAAACAAAAAAAAGTAGAAATATCAAC
GGAGATCACTGAACTTTTGACGCATGGTCGTGAAAAATTAGAGGAAAAATT
AAATTCAGGAGAGGTTATATATGGAATCAATACAGGATTTGGAGGGAATGC
CAATTTAGTTGTGCCATTTGAGAAAATCGCAGAGCATCAGCAAAATCTGTT
AACTTTTCTTTCTGCTGGTACTGGGGACTATATGTCCAAACCTTGTATTAA
AGCGTCACAATTTACTATGTTACTTTCTGTTTGCAAAGGTTGGTCTGCAAC
CAGACCAATTGTCGCTCAAGCAATTGTTGATCATATTAATCATGACATTGT
TCCTCTGGTTCCTCGCTATGGCTCAGTGGGTGCAAGCGGTGATTTAATTCC
TTTATCTTATATTGCACGAGCATTATGTGGTATCGGCAAAGTTTATTATAT
GGGCGCAGAAATTGACGCTGCTGAAGCAATTAAACGTGCAGGGTTGACACC
ATTATCGTTAAAAGCCAAAGAAGGTCTTGCTCTGATTAACGGCACCCGGGT
AATGTCAGGAATCAGTGCAATCACCGTCATTAAACTGGAAAAACTATTTAA
AGCCTCAATTTCTGCGATTGCCCTTGCTGTTGAAGCATTACTTGCATCTCA
TGAACATTATGATGCCCGGATTCAACAAGTAAAAAATCATCCTGGTCAAAA
CGCGGTGGCAAGTGCATTGCGTAATTTATTGGCAGGTTCAACGCAGGTTAA
TCTATTATCTGGGGTTAAAGAACAAGCCAATAAAGCTTGTCGTCATCAAGA
AATTACCCAACTAAATGATACCTTACAGGAAGTTTATTCAATTCGCTGTGC
ACCACAAGTATTAGGTATAGTGCCAGAATCTTTAGCTACCGCTCGGAAAAT
ATTGGAACGGGAAGTTATCTCAGCTAATGATAATCCATTGATAGATCCAGA
AAATGGCGATGTTCTACACGGTGGAAATTTTATGGGGCAATATGTCGCCCG
AACAATGGATGCATTAAAACTGGATATTGCTTTAATTGCCAATCATCTTCA
CGCCATTGTGGCTCTTATGATGGATAACCGTTTCTCTCGTGGATTACCTAA
TTCACTGAGTCCGACACCCGGCATGTATCAAGGTTTTAAAGGCGTCCAACT
TTCTCAAACCGCTTTAGTTGCTGCAATTCGCCATGATTGTGCTGCATCAGG
TATTCATACCCTCGCCACAGAACAATACAATCAAGATATTGTCAGTTTAGG
TCTGCATGCCGCTCAAGATGTTTTAGAGATGGAGCAGAAATTACGCAATAT
TGTTTCAATGACAATTCTGGTAGTTTGTCAGGCCATTCATCTTCGCGGCAA
TATTAGTGAAATTGCGCCTGAAACTGCTAAATTTTACCATGCAGTACGCGA
AATCAGTTCTCCTTTGATCACTGATCGTGCGTTGGATGAAGATATAATCCG
CATTGCGGATGCAATTATTAATGATCAACTTCCTCTGCCAGAAATCATGCT
GGAAGAATAAGCTTGGCGTAATCATGGTCATAGCTGTTTCCTGTGTGAAAT
TGTTATCCGCTCACAATTCCACACAACATACGAGCCGGAAGCATAAAGTGT
AAAGCCTGGGGTGCCTAATGAGTGAGCTAACTCACATTAATTGCGTTGCGC
TCACTGCCCGCTTTCCAGTCGGGAAACCTGTCGTGCCAGCTGCATTAATGA
ATCGGCCAACGCGCGGGGAGAGGCGGTTTGCGTATTGGGCGCTCTTCCGCT
TCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTA
TCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCA
CGTTAAGGGATTTTGGTCATGAGATTATCAAAAAGGATCTTCACCTAGATC
CTTTTAAATTAAAAATGAAGTTTTAAATCAATCTAAAGTATATATGAGTAA
ACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATA
ACTACGATACGGGAGGGCTTACCATCTGGCCCCAGTGCTGCAATGATACCG
CGAGACCCACGCTCACCGGCTCCAGATTTATCAGCAATAAACCAGCCAGCC
GGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGT
TTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACT
GGTGAGTACTCAACCAAGTCATTCTGAGAATAGTGTATGCGGCGACCGAGT
TGCTCTTGCCCGGCGTCAATACGGGATAATACCGCGCCACATAGCAGAACT
TTAAAAGTGCTCATCATTGGAAAACGTTCTTCGGGGCGAAAACTCTCAAGG
ATCTTACCGCTGTTGAGATCCAGTTCGATGTAACCCACTCGTGCACCCAAC
TGATCTTCAGCATCTTTTACTTTCACCAGCGTTTCTGGGTGAGCAAAAACA
GGAAGGCAAAATGCCGCAAAAAAGGGAATAAGGGCGACACGGAAATGTTGA
ATACTCATACTCTTCCTTTTTCAATATTATTGAAGCATTTATCAGGGTTAT
TGTCTCATGAGCGGATACATATTTGAATGTATTTAGAAAAATAAACAAATA
GGGGTTCCGCGCACATTTCCCCGAAAAGTGCCACCTGACGTCTAAGAAACC
ATTATTATCATGACATTAACCTATAAAAATAGGCGTATCACGAGGCCCTTT
CGTCTCGCGCGTTTCGGTGATGACGGTGAAAACCTCTGACACATGCAGCTC
CCGGAGACGGTCACAGCTTGTCTGTAAGCGGATGCCGGGAGCAGACAAGCC
CGTCAGGGCGCGTCAGCGGGTGTTGGCGGGTGTCGGGGCTGGCTTAACTAT
GCGGCATCAGAGCAGATTGTACTGAGAGTGCACCATATGCGGTGTGAAATA
CCGCACAGATGCGTAAGGAGAAAATACCGCATCAGGCGCCATTCGCCATTC
AGGCTGCGCAACTGTTGGGAAGGGCGATCGGTGCGGGCCTCTTCGCTATTA
CGCCAGCTGGCGAAAGGGGGATGTGCTGCAAGGCGATTAAGTTGGGTAACG
CCAGGGTTTTCCCAGTCACGACGTTGTAAAACGACGGCCAGTGAATTCGTT
AAGACCCACTTTCACATTTAAGTTGTTTTTCTAATCCGCATATGATCAATT
CAAGGCCGAATAAGAAGGCTGGCTCTGCACCTTGGTGATCAAATAATTCGA
TAGCTTGTCGTAATAATGGCGGCATACTATCAGTAGTAGGTGTTTCCCTTT
CTTCTTTAGCGACTTGATGCTCTTGATCTTCCAATACGCAACCTAAAGTAA
AATGCCCCACAGCGCTGAGTGCATATAATGCATTCTCTAGTGAAAAACCTT
GTTGGCATAAAAAGGCTAATTGATTTTCGAGAGTTTCATACTGTTTTTCTG
TAGGCCGTGTACCTAAATGTACTTTTGCTCCATCGCGATGACTTAGTAAAG
CACATCTAAAACTTTTAGCGTTATTACGTAAAAAATCTTGCCAGCTTTCCC
CTTCTAAAGGGCAAAAGTGAGTATGGTGCCTATCTAACATCTCAATGGCTA
AGGCGTCGAGCAAAGCCCGCTTATTTTTTACATGCCAATACAATGTAGGCT
GCTCTACACCTAGCTTCTGGGCGAGTTTACGGGTTGTTAAACCTTCGATTC
CGACCTCATTAAGCAGCTCTAATGCGCTGTTAATCACTTTACTTTTATCTA
ATCTAGACATCATTAATTCCTAATTTTTGTTGACACTCTATCATTGATAGA
GTTATTTTACCACTCCCTATCAGTGATAGAGAAAAGTGAA

TABLE 9
Nucleotide sequences of FNR promoter-PAL1 con-
struct, low-copy (SEQ ID NO: 26B)

CTCTTGATCGTTATCAATTCCCACGCTGTTTCAGAGCGTTACCTTGCCCTT
AAACATTAGCAATGTCGATTTATCAGAGGGCCGACAGGCTCCCACAGGAGA
AAACCGATGAAAACACTATCACAGGCCCAATCTAAAACTTCTTCACAGCAA
TTCAGCTTTACCGGGAACTCGTCTGCGAATGTAATTATCGGCAATCAAAAG
CTGACCATTAATGATGTAGCTCGCGTTGCCCGGAATGGCACTTTGGTGTCA
CTGACGAACAATACCGACATTCTGCAAGGTATTCAAGCTAGCTGCGATTAT
ATCAATAACGCCGTTGAATCTGGCGAGCCAATCTACGGGGTAACAAGCGGT
TTTGGTGGGATGGCGAACGTTGCCATTAGCCGTGAACAGGCGAGCGAACTT
CAGACCAACCTCGTTTGGTTCCTAAAGACAGGAGCTGGTAATAAGTTACCT
CTGGCTGACGTAAGAGCCGCGATGCTGCTTCGCGCTAATAGTCACATGCGC
GGCGCCAGTGGTATCCGTCTTGAGCTTATCAAGAGGATGGAAATCTTCCTC
AACGCGGGTGTCACACCATATGTTTATGAGTTTGGTAGTATCGGAGCCAGT
GGTGATCTTGTTCCCCTGAGTTATATTACGGGTTCATTGATTGGTTTAGAC
CCGTCCTTTAAAGTGGATTTTAACGGGAAAGAAATGGACGCCCCGACCGCT
TTACGACAGCTTAATCTGAGCCCACTTACTTTGCTCCCTAAAGAAGGTCTT
GCCATGATGAATGGCACCTCTGTGATGACTGGAATTGCCGCGAATTGTGTG
TATGACACGCAGATCCTAACGGCCATTGCCATGGGTGTTCACGCGTTGGAC
ATTCAAGCCCTGAATGGTACAAACCAGTCGTTTCATCCGTTTATCCATAAT
TCAAAACCCCATCCGGGACAGCTTTGGGCTGCTGATCAGATGATCTCACTC
CTGGCCAATAGTCAACTGGTTCGGGACGAGCTCGACGGCAAACATGATTAT
CGCGATCATGAGCTCATCCAGGACCGGTATTCACTTCGTTGTCTCCCACAA
TACCTGGGGCCTATCGTTGATGGTATATCTCAAATTGCGAAGCAAATTGAA
ATTGAGATCAATAGCGTAACCGACAACCCGCTTATCGATGTTGATAATCAG
GCCTCTTATCACGGTGGCAATTTTCTGGGCCAGTATGTTGGTATGGGGATG
GATCACCTGCGGTACTATATTGGGCTTCTGGCTAAACATCTTGATGTGCAG
ATTGCCTTATTAGCTTCACCAGAATTTTCAAATGGACTGCCGCCATCATTG
CTCGGTAACAGAGAAAGGAAAGTAAATATGGGCCTTAAGGGCCTTCAGATA
TGTGGTAACTCAATCATGCCCCTCCTGACCTTTTATGGGAACTCAATTGCT
GATCGTTTTCCGACACATGCTGAACAGTTTAACCAAAACATTAACTCACAG
GGCTATACATCCGCGACGTTAGCGCGTCGGTCCGTGGATATCTTCCAGAAT
TATGTTGCTATCGCTCTGATGTTCGGCGTACAGGCCGTTGATTTGCGCACT
TATAAAAAAACCGGTCACTACGATGCTCGGGCTTGCCTGTCGCCTGCCACC
GAGCGGCTTTATAGCGCCGTACGTCATGTTGTGGGTCAGAAACCGACGTCG
GACCGCCCCTATATTTGGAATGATAATGAACAAGGGCTGGATGAACACATC
GCCCGGATATCTGCCGATATTGCCGCCGGAGGTGTCATCGTCCAGGCGGTA
CAAGACATACTTCCTTGCCTGCATTAAGCTTGGCGTAATCATGGTCATAGC
TGTTTCCTGTGTGAAATTGTTATCCGCTCACAATTCCACACAACATACGAG
CCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTC
GGCTGCGGCGAGCGGTATCAGCTCACTCAAAGGCGGTAGTACGGGTTTTGC
TGCCCGCAAACGGGCTGTTCTGGTGTTGCTAGTTTGTTATCAGAATCGCAG
ATCCGGCTTCAGGTTTGCCGGCTGAAAGCGCTATTTCTTCCAGAATTGCCA
TGATTTTTTCCCCACGGGAGGCGTCACTGGCTCCCGTGTTGTCGGCAGCTT
TGATTCGATAAGCAGCATCGCCTGTTTCAGGCTGTCTATGTGTGACTGTTG
AGCTGTAACAAGTTGTCTCAGGTGTTCAATTTCATGTTCTAGTTGCTTTGT
TTTACTGGTTTCACCTGTTCTATTAGGTGTTACATGCTGTTCATCTGTTAC
ATTGTCGATCTGTTCATGGTGAACAGCTTTAAATGCACCAAAAACTCGTAA
AAGCTCTGATGTATCTATCTTTTTTACACCGTTTTCATCTGTGCATATGGA
CAGTTTTCCCTTTGATATCTAACGGTGAACAGTTGTTCTACTTTTGTTTGT
TAGTCTTGATGCTTCACTGATAGATACAAGAGCCATAAGAACCTCAGATCC
TTCCGTATTTAGCCAGTATGTTCTCTAGTGTGGTTCGTTGTTTTTGCGTGA
GCCATGAGAACGAACCATTGAGATCATGCTTACTTTGCATGTCACTCAAAA
ATTTTGCCTCAAAACTGGTGAGCTGAATTTTTGCAGTTAAAGCATCGTGTA
GTGTTTTTCTTAGTCCGTTACGTAGGTAGGAATCTGATGTAATGGTTGTTG
GTATTTTGTCACCATTCATTTTTATCTGGTTGTTCTCAAGTTCGGTTACGA
GATCCATTTGTCTATCTAGTTCAACTTGGAAAATCAACGTATCAGTCGGGC
GGCCTCGCTTATCAACCACCAATTTCATATTGCTGTAAGTGTTTAAATCTT
TACTTATTGGTTTCAAAACCCATTGGTTAAGCCTTTTAAACTCATGGTAGT
TATTTTCAAGCATTAACATGAACTTAAATTCATCAAGGCTAATCTCTATAT
TTGCCTTGTGAGTTTTCTTTTGTGTTAGTTCTTTTAATAACCACTCATAAA
TCCTCATAGAGTATTTGTTTTCAAAAGACTTAACATGTTCCAGATTATATT
TTATGAATTTTTTTAACTGGAAAAGATAAGGCAATATCTCTTCACTAAAAA
CTAATTCTAATTTTTCGCTTGAGAACTTGGCATAGTTTGTCCACTGGAAAA
TCTCAAAGCCTTTAACCAAAGGATTCCTGATTTCCACAGTTCTCGTCATCA
GCTCTCTGGTTGCTTTAGCTAATACACCATAAGCATTTTCCCTACTGATGT
TCATCATCTGAGCGTATTGGTTATAAGTGAACGATACCGTCCGTTCTTTCC
TTGTAGGGTTTTCAATCGTGGGGTTGAGTAGTGCCACACAGCATAAAATTA
GCTTGGTTTCATGCTCCGTTAAGTCATAGCGACTAATCGCTAGTTCATTTG
CTTTGAAAACAACTAATTCAGACATACATCTCAATTGGTCTAGGTGATTTT
AATCACTATACCAATTGAGATGGGCTAGTCAATGATAATTACTAGTCCTTT
TCCTTTGAGTTGTGGGTATCTGTAAATTCTGCTAGACCTTTGCTGGAAAAC
TTGTAAATTCTGCTAGACCCTCTGTAAATTCCGCTAGACCTTTGTGTGTTT
TTTTTGTTTATATTCAAGTGGTTATAATTTATAGAATAAAGAAAGAATAAA
AAAAGATAAAAAGAATAGATCCCAGCCCTGTGTATAACTCACTACTTTAGT
CAGTTCCGCAGTATTACAAAAGGATGTCGCAAACGCTGTTTGCTCCTCTAC
AAAACAGACCTTAAAACCCTAAAGGCTTAAGTAGCACCCTCGCAAGCTCGG
GCAAATCGCTGAATATTCCTTTTGTCTCCGACCATCAGGCACCTGAGTCGC
TGTCTTTTTCGTGACATTCAGTTCGCTGCGCTCACGGCTCTGGCAGTGAAT
GGGGGTAAATGGCACTACAGGCGCCTTTTATGGATTCATGCAAGGAAACTA
CCCATAATACAAGAAAAGCCCGTCACGGGCTTCTCAGGGCGTTTTATGGCG
GGTCTGCTATGTGGTGCTATCTGACTTTTTGCTGTTCAGCAGTTCCTGCCC
TCTGATTTTCCAGTCTGACCACTTCGGATTATCCCGTGACAGGTCATTCAG
ACTGGCTAATGCACCCAGTAAGGCAGCGGTATCATCAACAGGCTTACCCGT
CTTACTGTCTTTTCTACGGGGTCTGACGCTCAGTGGAACGAAAACTCACGT
TAAGGGATTTTGGTCATGAGATTATCAAAAAGGATCTTCACCTAGATCCTT
TTAAATTAAAAATGAAGTTTTAAATCAATCTAAAGTATATATGAGTAAACT
TGGTCTGACAGTTACCAATGCTTAATCAGTGAGGCACCTATCTCAGCGATC
TGTCTATTTCGTTCATCCATAGTTGCCTGACTCCCCGTCGTGTAGATAACT
ACGATACGGGAGGGCTTACCATCTGGCCCCAGTGCTGCAATGATACCGCGA
GACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTTT
GGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTG
CATAATTCTCTTACTGTCATGCCATCCGTAAGATGCTTTTCTGTGACTGGT
GAGTACTCAACCAAGTCATTCTGAGAATAGTGTATGCGGCGACCGAGTTGC
TCTTGCCCGGCGTCAATACGGGATAATACCGCGCCACATAGCAGAACTTTA
AAAGTGCTCATCATTGGAAAACGTTCTTCGGGGCGAAAACTCTCAAGGATC
TTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATA
CTCATACTCTTCCTTTTTCAATATTATTGAAGCATTTATCAGGGTTATTGT
CTCATGAGCGGATACATATTTGAATGTATTTAGAAAAATAAACAAATAGGG
GTTCCGCGCACATTTCCCCGAAAAGTGCCACCTGACGTCTAAGAAACCATT
ATTATCATGACATTAACCTATAAAAATAGGCGTATCACGAGGCCCTTTCGT
CTCGCGCGTTTCGGTGATGACGGTGAAAACCTCTGACACATGCAGCTCCCG
GAGACGGTCACAGCTTGTCTGTAAGCGGATGCCGGGAGCAGACAAGCCCGT
CAGGGCGCGTCAGCGGGTGTTGGCGGGTGTCGGGGCTGGCTTAACTATGCG
GCATCAGAGCAGATTGTACTGAGAGTGCACCATATGCGGTGTGAAATACCG
CACAGATGCGTAAGGAGAAAATACCGCATCAGGCGCCATTCGCCATTCAGG
CTGCGCAACTGTTGGGAAGGGCGATCGGTGCGGGCCTCTTCGCTATTACGC
CAGCTGGCGAAAGGGGGATGTGCTGCAAGGCGATTAAGTTGGGTAACGCCA
GGGTTTTCCCAGTCACGACGTTGTAAAACGACGGCCAGTGAATTCG

TABLE 10
Nucleotide sequences of FNR promoter-PAL3 con-
struct, low-copy (SEQ ID NO: 27B)

CTCTTGATCGTTATCAATTCCCACGCTGTTTCAGAGCGTTACCTTGCCCTT
AAACATTAGCAATGTCGATTTATCAGAGGGCCGACAGGCTCCCACAGGAGA
AAACCGATGAAAGCTAAAGATGTTCAGCCAACCATTATTATTAATAAAAAT
GGCCTTATCTCTTTGGAAGATATCTATGACATTGCGATAAAACAAAAAAAA
GTAGAAATATCAACGGAGATCACTGAACTTTTGACGCATGGTCGTGAAAAA
TTAGAGGAAAAATTAAATTCAGGAGAGGTTATATATGGAATCAATACAGGA
TTTGGAGGGAATGCCAATTTAGTTGTGCCATTTGAGAAAATCGCAGAGCAT
CAGCAAAATCTGTTAACTTTTCTTTCTGCTGGTACTGGGGACTATATGTCC
AAACCTTGTATTAAAGCGTCACAATTTACTATGTTACTTTCTGTTTGCAAA
GGTTGGTCTGCAACCAGACCAATTGTCGCTCAAGCAATTGTTGATCATATT
AATCATGACATTGTTCCTCTGGTTCCTCGCTATGGCTCAGTGGGTGCAAGC
GGTGATTTAATTCCTTTATCTTATATTGCACGAGCATTATGTGGTATCGGC
AAAGTTTATTATATGGGCGCAGAAATTGACGCTGCTGAAGCAATTAAACGT
GCAGGGTTGACACCATTATCGTTAAAAGCCAAAGAAGGTCTTGCTCTGATT
AACGGCACCCGGGTAATGTCAGGAATCAGTGCAATCACCGTCATTAAACTG
GAAAAACTATTTAAAGCCTCAATTTCTGCGATTGCCCTTGCTGTTGAAGCA
TTACTTGCATCTCATGAACATTATGATGCCCGGATTCAACAAGTAAAAAAT
CATCCTGGTCAAAACGCGGTGGCAAGTGCATTGCGTAATTTATTGGCAGGT
TCAACGCAGGTTAATCTATTATCTGGGGTTAAAGAACAAGCCAATAAAGCT
TGTCGTCATCAAGAAATTACCCAACTAAATGATACCTTACAGGAAGTTTAT
TCAATTCGCTGTGCACCACAAGTATTAGGTATAGTGCCAGAATCTTTAGCT
ACCGCTCGGAAAATATTGGAACGGGAAGTTATCTCAGCTAATGATAATCCA
TTGATAGATCCAGAAAATGGCGATGTTCTACACGGTGGAAATTTTATGGGG
CAATATGTCGCCCGAACAATGGATGCATTAAAACTGGATATTGCTTTAATT
GCCAATCATCTTCACGCCATTGTGGCTCTTATGATGGATAACCGTTTCTCT
CGTGGATTACCTAATTCACTGAGTCCGACACCCGGCATGTATCAAGGTTTT
AAAGGCGTCCAACTTTCTCAAACCGCTTTAGTTGCTGCAATTCGCCATGAT
TGTGCTGCATCAGGTATTCATACCCTCGCCACAGAACAATACAATCAAGAT
ATTGTCAGTTTAGGTCTGCATGCCGCTCAAGATGTTTTAGAGATGGAGCAG
AAATTACGCAATATTGTTTCAATGACAATTCTGGTAGTTTGTCAGGCCATT
CATCTTCGCGGCAATATTAGTGAAATTGCGCCTGAAACTGCTAAATTTTAC
CATGCAGTACGCGAAATCAGTTCTCCTTTGATCACTGATCGTGCGTTGGAT
GAAGATATAATCCGCATTGCGGATGCAATTATTAATGATCAACTTCCTCTG
CCAGAAATCATGCTGGAAGAATAAGCTTGGCGTAATCATGGTCATAGCTGT
TTCCTGTGTGAAATTGTTATCCGCTCACAATTCCACACAACATACGAGCCG
GAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTG
GGCGCTCTTCCGCTTCCTCGCTCACTGACTCGCTGCGCTCGGTCGTTCGGC
TGCGGCGAGCGGTATCAGCTCACTCAAAGGCGGTAGTACGGGTTTTGCTGC
CCGCAAACGGGCTGTTCTGGTGTTGCTAGTTTGTTATCAGAATCGCAGATC
CGGCTTCAGGTTTGCCGGCTGAAAGCGCTATTTCTTCCAGAATTGCCATGA
TTTTTTCCCCACGGGAGGCGTCACTGGCTCCCGTGTTGTCGGCAGCTTTGA
TTCGATAAGCAGCATCGCCTGTTTCAGGCTGTCTATGTGTGACTGTTGAGC
TGTAACAAGTTGTCTCAGGTGTTCAATTTCATGTTCTAGTTGCTTTGTTTT
ACTGGTTTCACCTGTTCTATTAGGTGTTACATGCTGTTCATCTGTTACATT
GTCGATCTGTTCATGGTGAACAGCTTTAAATGCACCAAAAACTCGTAAAAG
CTCTGATGTATCTATCTTTTTTACACCGTTTTCATCTGTGCATATGGACAG
TTTTCCCTTTGATATCTAACGGTGAACAGTTGTTCTACTTTTGTTTGTTAG
TCTTGATGCTTCACTGATAGATACAAGAGCCATAAGAACCTCAGATCCTTC
CGTATTTAGCCAGTATGTTCTCTAGTGTGGTTCGTTGTTTTTGCGTGAGCC
ATGAGAACGAACCATTGAGATCATGCTTACTTTGCATGTCACTCAAAAATT
TTGCCTCAAAACTGGTGAGCTGAATTTTTGCAGTTAAAGCATCGTGTAGTG
TTTTTCTTAGTCCGTTACGTAGGTAGGAATCTGATGTAATGGTTGTTGGTA
TTTTGTCACCATTCATTTTTATCTGGTTGTTCTCAAGTTCGGTTACGAGAT
CCATTTGTCTATCTAGTTCAACTTGGAAAATCAACGTATCAGTCGGGCGGC
CTCGCTTATCAACCACCAATTTCATATTGCTGTAAGTGTTTAAATCTTTAC
TTATTGGTTTCAAAACCCATTGGTTAAGCCTTTTAAACTCATGGTAGTTAT
TTTCAAGCATTAACATGAACTTAAATTCATCAAGGCTAATCTCTATATTTG
CCTTGTGAGTTTTCTTTTGTGTTAGTTCTTTTAATAACCACTCATAAATCC
TCATAGAGTATTTGTTTTCAAAAGACTTAACATGTTCCAGATTATATTTTA
TGAATTTTTTTAACTGGAAAAGATAAGGCAATATCTCTTCACTAAAAACTA
ATTCTAATTTTTCGCTTGAGAACTTGGCATAGTTTGTCCACTGGAAAATCT
CAAAGCCTTTAACCAAAGGATTCCTGATTTCCACAGTTCTCGTCATCAGCT
CTCTGGTTGCTTTAGCTAATACACCATAAGCATTTTCCCTACTGATGTTCA
TCATCTGAGCGTATTGGTTATAAGTGAACGATACCGTCCGTTCTTTCCTTG
TAGGGTTTTCAATCGTGGGGTTGAGTAGTGCCACACAGCATAAAATTAGCT
TGGTTTCATGCTCCGTTAAGTCATAGCGACTAATCGCTAGTTCATTTGCTT
TGAAAACAACTAATTCAGACATACATCTCAATTGGTCTAGGTGATTTTAAT
CACTATACCAATTGAGATGGGCTAGTCAATGATAATTACTAGTCCTTTTCC
TTTGAGTTGTGGGTATCTGTAAATTCTGCTAGACCTTTGCTGGAAAACTTG
TAAATTCTGCTAGACCCTCTGTAAATTCCGCTAGACCTTTGTGTGTTTTTT
TTGTTTATATTCAAGTGGTTATAATTTATAGAATAAAGAAAGAATAAAAAA
AGATAAAAAGAATAGATCCCAGCCCTGTGTATAACTCACTACTTTAGTCAG
TTCCGCAGTATTACAAAAGGATGTCGCAAACGCTGTTTGCTCCTCTACAAA
ACAGACCTTAAAACCCTAAAGGCTTAAGTAGCACCCTCGCAAGCTCGGGCA
AATCGCTGAATATTCCTTTTGTCTCCGACCATCAGGCACCTGAGTCGCTGT
CTTTTTCGTGACATTCAGTTCGCTGCGCTCACGGCTCTGGCAGTGAATGGG
GGTAAATGGCACTACAGGCGCCTTTTATGGATTCATGCAAGGAAACTACCC
ATAATACAAGAAAAGCCCGTCACGGGCTTCTCAGGGCGTTTTATGGCGGGT
CTGCTATGTGGTGCTATCTGACTTTTTGCTGTTCAGCAGTTCCTGCCCTCT
GATTTTCCAGTCTGACCACTTCGGATTATCCCGTGACAGGTCATTCAGACT
GGCTAATGCACCCAGTAAGGCAGCGGTATCATCAACAGGCTTACCCGTCTT
ACTGTCTTTTCTACGGGGTCTGACGCTCAGTGGAACGAAAACTCACGTTAA
GGGATTTTGGTCATGAGATTATCAAAAAGGATCTTCACCTAGATCCTTTTA
AATTAAAAATGAAGTTTTAAATCAATCTAAAGTATATATGAGTAAACTTGG
TCTGACAGTTACCAATGCTTAATCAGTGAGGCACCTATCTCAGCGATCTGT
CTATTTCGTTCATCCATAGTTGCCTGACTCCCCGTCGTGTAGATAACTACG
ATACGGGAGGGCTTACCATCTGGCCCCAGTGCTGCAATGATACCGCGAGAC
CCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTC
AGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAG
TACTCAACCAAGTCATTCTGAGAATAGTGTATGCGGCGACCGAGTTGCTCT
TGCCCGGCGTCAATACGGGATAATACCGCGCCACATAGCAGAACTTTAAAA
GTGCTCATCATTGGAAAACGTTCTTCGGGGCGAAAACTCTCAAGGATCTTA
CCGCTGTTGAGATCCAGTTCGATGTAACCCACTCGTGCACCCAACTGATCT
TCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTC
ATACTCTTCCTTTTTCAATATTATTGAAGCATTTATCAGGGTTATTGTCTC
ATGAGCGGATACATATTTGAATGTATTTAGAAAAATAAACAAATAGGGGTT
CCGCGCACATTTCCCCGAAAAGTGCCACCTGACGTCTAAGAAACCATTATT
ATCATGACATTAACCTATAAAAATAGGCGTATCACGAGGCCCTTTCGTCTC
GCGCGTTTCGGTGATGACGGTGAAAACCTCTGACACATGCAGCTCCCGGAG
ACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCA
TCAGAGCAGATTGTACTGAGAGTGCACCATATGCGGTGTGAAATACCGCAC
AGATGCGTAAGGAGAAAATACCGCATCAGGCGCCATTCGCCATTCAGGCTG
CGCAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAGGG
TTTTCCCAGTCACGACGTTGTAAAACGACGGCCAGTGAATTCG

TABLE 11
Nucleotide sequences of Tet promoter-PAL1 con-
struct, low-copy (SEQ ID NO: 28B)

ACCACTCCCTATCAGTGATAGAGAAAAGTGAACTCTAGAAATAATTTTGTT
TAACTTTAAGAAGGAGATATACATATGAAAACACTATCACAGGCCCAATCT
AAAACTTCTTCACAGCAATTCAGCTTTACCGGGAACTCGTCTGCGAATGTA
ATTATCGGCAATCAAAAGCTGACCATTAATGATGTAGCTCGCGTTGCCCGG
AATGGCACTTTGGTGTCACTGACGAACAATACCGACATTCTGCAAGGTATT
CAAGCTAGCTGCGATTATATCAATAACGCCGTTGAATCTGGCGAGCCAATC
TACGGGGTAACAAGCGGTTTTGGTGGGATGGCGAACGTTGCCATTAGCCGT
GAACAGGCGAGCGAACTTCAGACCAACCTCGTTTGGTTCCTAAAGACAGGA
GCTGGTAATAAGTTACCTCTGGCTGACGTAAGAGCCGCGATGCTGCTTCGC
GCTAATAGTCACATGCGCGGCGCCAGTGGTATCCGTCTTGAGCTTATCAAG
AGGATGGAAATCTTCCTCAACGCGGGTGTCACACCATATGTTTATGAGTTT
GGTAGTATCGGAGCCAGTGGTGATCTTGTTCCCCTGAGTTATATTACGGGT
TCATTGATTGGTTTAGACCCGTCCTTTAAAGTGGATTTTAACGGGAAAGAA
ATGGACGCCCCGACCGCTTTACGACAGCTTAATCTGAGCCCACTTACTTTG
CTCCCTAAAGAAGGTCTTGCCATGATGAATGGCACCTCTGTGATGACTGGA
ATTGCCGCGAATTGTGTGTATGACACGCAGATCCTAACGGCCATTGCCATG
GGTGTTCACGCGTTGGACATTCAAGCCCTGAATGGTACAAACCAGTCGTTT
CATCCGTTTATCCATAATTCAAAACCCCATCCGGGACAGCTTTGGGCTGCT
GATCAGATGATCTCACTCCTGGCCAATAGTCAACTGGTTCGGGACGAGCTC
GACGGCAAACATGATTATCGCGATCATGAGCTCATCCAGGACCGGTATTCA
CTTCGTTGTCTCCCACAATACCTGGGGCCTATCGTTGATGGTATATCTCAA
ATTGCGAAGCAAATTGAAATTGAGATCAATAGCGTAACCGACAACCCGCTT
ATCGATGTTGATAATCAGGCCTCTTATCACGGTGGCAATTTTCTGGGCCAG
TATGTTGGTATGGGGATGGATCACCTGCGGTACTATATTGGGCTTCTGGCT
AAACATCTTGATGTGCAGATTGCCTTATTAGCTTCACCAGAATTTTCAAAT
GGACTGCCGCCATCATTGCTCGGTAACAGAGAAAGGAAAGTAAATATGGGC
CTTAAGGGCCTTCAGATATGTGGTAACTCAATCATGCCCCTCCTGACCTTT
TATGGGAACTCAATTGCTGATCGTTTTCCGACACATGCTGAACAGTTTAAC
CAAAACATTAACTCACAGGGCTATACATCCGCGACGTTAGCGCGTCGGTCC
GTGGATATCTTCCAGAATTATGTTGCTATCGCTCTGATGTTCGGCGTACAG
GCCGTTGATTTGCGCACTTATAAAAAAACCGGTCACTACGATGCTCGGGCT
TGCCTGTCGCCTGCCACCGAGCGGCTTTATAGCGCCGTACGTCATGTTGTG
GGTCAGAAACCGACGTCGGACCGCCCCTATATTTGGAATGATAATGAACAA
GGGCTGGATGAACACATCGCCCGGATATCTGCCGATATTGCCGCCGGAGGT
GTCATCGTCCAGGCGGTACAAGACATACTTCCTTGCCTGCATTAAGCTTGG
CGTAATCATGGTCATAGCTGTTTCCTGTGTGAAATTGTTATCCGCTCACAA
TTCCACACAACATACGAGCCGGAAGCATAAAGTGTAAAGCCTGGGGTGCCT
AATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAGG
CGGTAGTACGGGTTTTGCTGCCCGCAAACGGGCTGTTCTGGTGTTGCTAGT
TTGTTATCAGAATCGCAGATCCGGCTTCAGGTTTGCCGGCTGAAAGCGCTA
TTTCTTCCAGAATTGCCATGATTTTTTCCCCACGGGAGGCGTCACTGGCTC
CCGTGTTGTCGGCAGCTTTGATTCGATAAGCAGCATCGCCTGTTTCAGGCT
GTCTATGTGTGACTGTTGAGCTGTAACAAGTTGTCTCAGGTGTTCAATTTC
ATGTTCTAGTTGCTTTGTTTTACTGGTTTCACCTGTTCTATTAGGTGTTAC
ATGCTGTTCATCTGTTACATTGTCGATCTGTTCATGGTGAACAGCTTTAAA
TGCACCAAAAACTCGTAAAAGCTCTGATGTATCTATCTTTTTTACACCGTT
TTCATCTGTGCATATGGACAGTTTTCCCTTTGATATCTAACGGTGAACAGT
TGTTCTACTTTTGTTTGTTAGTCTTGATGCTTCACTGATAGATACAAGAGC
CATAAGAACCTCAGATCCTTCCGTATTTAGCCAGTATGTTCTCTAGTGTGG
TTCGTTGTTTTTGCGTGAGCCATGAGAACGAACCATTGAGATCATGCTTAC
TTTGCATGTCACTCAAAAATTTTGCCTCAAAACTGGTGAGCTGAATTTTTG
CAGTTAAAGCATCGTGTAGTGTTTTTCTTAGTCCGTTACGTAGGTAGGAAT
CTGATGTAATGGTTGTTGGTATTTTGTCACCATTCATTTTTATCTGGTTGT
TCTCAAGTTCGGTTACGAGATCCATTTGTCTATCTAGTTCAACTTGGAAAA
TCAACGTATCAGTCGGGCGGCCTCGCTTATCAACCACCAATTTCATATTGC
TGTAAGTGTTTAAATCTTTACTTATTGGTTTCAAAACCCATTGGTTAAGCC
TTTTAAACTCATGGTAGTTATTTTCAAGCATTAACATGAACTTAAATTCAT
CAAGGCTAATCTCTATATTTGCCTTGTGAGTTTTCTTTTGTGTTAGTTCTT
TTAATAACCACTCATAAATCCTCATAGAGTATTTGTTTTCAAAAGACTTAA
CATGTTCCAGATTATATTTTATGAATTTTTTTAACTGGAAAAGATAAGGCA
ATATCTCTTCACTAAAAACTAATTCTAATTTTTCGCTTGAGAACTTGGCAT
AGTTTGTCCACTGGAAAATCTCAAAGCCTTTAACCAAAGGATTCCTGATTT
CCACAGTTCTCGTCATCAGCTCTCTGGTTGCTTTAGCTAATACACCATAAG
CATTTTCCCTACTGATGTTCATCATCTGAGCGTATTGGTTATAAGTGAACG
ATACCGTCCGTTCTTTCCTTGTAGGGTTTTCAATCGTGGGGTTGAGTAGTG
CCACACAGCATAAAATTAGCTTGGTTTCATGCTCCGTTAAGTCATAGCGAC
TAATCGCTAGTTCATTTGCTTTGAAAACAACTAATTCAGACATACATCTCA
ATTGGTCTAGGTGATTTTAATCACTATACCAATTGAGATGGGCTAGTCAAT
GATAATTACTAGTCCTTTTCCTTTGAGTTGTGGGTATCTGTAAATTCTGCT
AGACCTTTGCTGGAAAACTTGTAAATTCTGCTAGACCCTCTGTAAATTCCG
CTAGACCTTTGTGTGTTTTTTTTGTTTATATTCAAGTGGTTATAATTTATA
GAATAAAGAAAGAATAAAAAAAGATAAAAAGAATAGATCCCAGCCCTGTGT
ATAACTCACTACTTTAGTCAGTTCCGCAGTATTACAAAAGGATGTCGCAAA
CGCTGTTTGCTCCTCTACAAAACAGACCTTAAAACCCTAAAGGCTTAAGTA
GCACCCTCGCAAGCTCGGGCAAATCGCTGAATATTCCTTTTGTCTCCGACC
ATCAGGCACCTGAGTCGCTGTCTTTTTCGTGACATTCAGTTCGCTGCGCTC
ACGGCTCTGGCAGTGAATGGGGGTAAATGGCACTACAGGCGCCTTTTATGG
ATTCATGCAAGGAAACTACCCATAATACAAGAAAAGCCCGTCACGGGCTTC
TCAGGGCGTTTTATGGCGGGTCTGCTATGTGGTGCTATCTGACTTTTTGCT
GTTCAGCAGTTCCTGCCCTCTGATTTTCCAGTCTGACCACTTCGGATTATC
CCGTGACAGGTCATTCAGACTGGCTAATGCACCCAGTAAGGCAGCGGTATC
ATCAACAGGCTTACCCGTCTTACTGTCTTTTCTACGGGGTCTGACGCTCAG
TGGAACGAAAACTCACGTTAAGGGATTTTGGTCATGAGATTATCAAAAAGG
ATCTTCACCTAGATCCTTTTAAATTAAAAATGAAGTTTTAAATCAATCTAA
AGTATATATGAGTAAACTTGGTCTGACAGTTACCAATGCTTAATCAGTGAG
GCACCTATCTCAGCGATCTGTCTATTTCGTTCATCCATAGTTGCCTGACTC
CCCGTCGTGTAGATAACTACGATACGGGAGGGCTTACCATCTGGCCCCAGT
GCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGA
TGCTTTTCTGTGACTGGTGAGTACTCAACCAAGTCATTCTGAGAATAGTGT
ATGCGGCGACCGAGTTGCTCTTGCCCGGCGTCAATACGGGATAATACCGCG
CCACATAGCAGAACTTTAAAAGTGCTCATCATTGGAAAACGTTCTTCGGGG
CGAAAACTCTCAAGGATCTTACCGCTGTTGAGATCCAGTTCGATGTAACCC
ACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCG
ACACGGAAATGTTGAATACTCATACTCTTCCTTTTTCAATATTATTGAAGC
ATTTATCAGGGTTATTGTCTCATGAGCGGATACATATTTGAATGTATTTAG
AAAAATAAACAAATAGGGGTTCCGCGCACATTTCCCCGAAAAGTGCCACCT
GACGTCTAAGAAACCATTATTATCATGACATTAACCTATAAAAATAGGCGT
ATCACGAGGCCCTTTCGTCTCGCGCGTTTCGGTGATGACGGTGAAAACCTC
TGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGG
GGCTGGCTTAACTATGCGGCATCAGAGCAGATTGTACTGAGAGTGCACCAT
ATGCGGTGTGAAATACCGCACAGATGCGTAAGGAGAAAATACCGCATCAGG
CGCCATTCGCCATTCAGGCTGCGCAACTGTTGGGAAGGGCGATCGGTGCGG
GCCTCTTCGCTATTACGCCAGCTGGCGAAAGGGGGATGTGCTGCAAGGCGA
TTAAGTTGGGTAACGCCAGGGTTTTCCCAGTCACGACGTTGTAAAACGACG
GCCAGTGAATTCGTTAAGACCCACTTTCACATTTAAGTTGTTTTTCTAATC
CGCATATGATCAATTCAAGGCCGAATAAGAAGGCTGGCTCTGCACCTTGGT
GATCAAATAATTCGATAGCTTGTCGTAATAATGGCGGCATACTATCAGTAG
TAGGTGTTTCCCTTTCTTCTTTAGCGACTTGATGCTCTTGATCTTCCAATA
CGCAACCTAAAGTAAAATGCCCCACAGCGCTGAGTGCATATAATGCATTCT
CTAGTGAAAAACCTTGTTGGCATAAAAAGGCTAATTGATTTTCGAGAGTTT
CATACTGTTTTTCTGTAGGCCGTGTACCTAAATGTACTTTTGCTCCATCGC
GATGACTTAGTAAAGCACATCTAAAACTTTTAGCGTTATTACGTAAAAAAT
CTTGCCAGCTTTCCCCTTCTAAAGGGCAAAAGTGAGTATGGTGCCTATCTA
ACATCTCAATGGCTAAGGCGTCGAGCAAAGCCCGCTTATTTTTTACATGCC
AATACAATGTAGGCTGCTCTACACCTAGCTTCTGGGCGAGTTTACGGGTTG
TTAAACCTTCGATTCCGACCTCATTAAGCAGCTCTAATGCGCTGTTAATCA
CTTTACTTTTATCTAATCTAGACATCATTAATTCCTAATTTTTGTTGACAC
TCTATCATTGATAGAGTTATTTT

TABLE 12
Nucleotide sequences of Tet promoter-PAL3 con-
struct, low-copy (SEQ ID NO: 29B)

ACCACTCCCTATCAGTGATAGAGAAAAGTGAACTCTAGAAATAATTTTGTT
TAACTTTAAGAAGGAGATATACATATGAAAGCTAAAGATGTTCAGCCAACC
ATTATTATTAATAAAAATGGCCTTATCTCTTTGGAAGATATCTATGACATT
GCGATAAAACAAAAAAAAGTAGAAATATCAACGGAGATCACTGAACTTTTG
ACGCATGGTCGTGAAAAATTAGAGGAAAAATTAAATTCAGGAGAGGTTATA
TATGGAATCAATACAGGATTTGGAGGGAATGCCAATTTAGTTGTGCCATTT
GAGAAAATCGCAGAGCATCAGCAAAATCTGTTAACTTTTCTTTCTGCTGGT
ACTGGGGACTATATGTCCAAACCTTGTATTAAAGCGTCACAATTTACTATG
TTACTTTCTGTTTGCAAAGGTTGGTCTGCAACCAGACCAATTGTCGCTCAA
GCAATTGTTGATCATATTAATCATGACATTGTTCCTCTGGTTCCTCGCTAT
GGCTCAGTGGGTGCAAGCGGTGATTTAATTCCTTTATCTTATATTGCACGA
GCATTATGTGGTATCGGCAAAGTTTATTATATGGGCGCAGAAATTGACGCT
GCTGAAGCAATTAAACGTGCAGGGTTGACACCATTATCGTTAAAAGCCAAA
GAAGGTCTTGCTCTGATTAACGGCACCCGGGTAATGTCAGGAATCAGTGCA
ATCACCGTCATTAAACTGGAAAAACTATTTAAAGCCTCAATTTCTGCGATT
GCCCTTGCTGTTGAAGCATTACTTGCATCTCATGAACATTATGATGCCCGG
ATTCAACAAGTAAAAAATCATCCTGGTCAAAACGCGGTGGCAAGTGCATTG
CGTAATTTATTGGCAGGTTCAACGCAGGTTAATCTATTATCTGGGGTTAAA
GAACAAGCCAATAAAGCTTGTCGTCATCAAGAAATTACCCAACTAAATGAT
ACCTTACAGGAAGTTTATTCAATTCGCTGTGCACCACAAGTATTAGGTATA
GTGCCAGAATCTTTAGCTACCGCTCGGAAAATATTGGAACGGGAAGTTATC
TCAGCTAATGATAATCCATTGATAGATCCAGAAAATGGCGATGTTCTACAC
GGTGGAAATTTTATGGGGCAATATGTCGCCCGAACAATGGATGCATTAAAA
CTGGATATTGCTTTAATTGCCAATCATCTTCACGCCATTGTGGCTCTTATG
ATGGATAACCGTTTCTCTCGTGGATTACCTAATTCACTGAGTCCGACACCC
GGCATGTATCAAGGTTTTAAAGGCGTCCAACTTTCTCAAACCGCTTTAGTT
GCTGCAATTCGCCATGATTGTGCTGCATCAGGTATTCATACCCTCGCCACA
GAACAATACAATCAAGATATTGTCAGTTTAGGTCTGCATGCCGCTCAAGAT
GTTTTAGAGATGGAGCAGAAATTACGCAATATTGTTTCAATGACAATTCTG
GTAGTTTGTCAGGCCATTCATCTTCGCGGCAATATTAGTGAAATTGCGCCT
GAAACTGCTAAATTTTACCATGCAGTACGCGAAATCAGTTCTCCTTTGATC
ACTGATCGTGCGTTGGATGAAGATATAATCCGCATTGCGGATGCAATTATT
AATGATCAACTTCCTCTGCCAGAAATCATGCTGGAAGAATAAGCTTGGCGT
AATCATGGTCATAGCTGTTTCCTGTGTGAAATTGTTATCCGCTCACAATTC
CACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGT
CGGGAAACCTGTCGTGCCAGCTGCATTAATGAATCGGCCAACGCGCGGGGA
GAGGCGGTTTGCGTATTGGGCGCTCTTCCGCTTCCTCGCTCACTGACTCGC
TGCGCTCGGTCGTTCGGCTGCGGCGAGCGGTATCAGCTCACTCAAAGGCGG
TAGTACGGGTTTTGCTGCCCGCAAACGGGCTGTTCTGGTGTTGCTAGTTTG
TTATCAGAATCGCAGATCCGGCTTCAGGTTTGCCGGCTGAAAGCGCTATTT
CTTCCAGAATTGCCATGATTTTTTCCCCACGGGAGGCGTCACTGGCTCCCG
TGTTGTCGGCAGCTTTGATTCGATAAGCAGCATCGCCTGTTTCAGGCTGTC
TATGTGTGACTGTTGAGCTGTAACAAGTTGTCTCAGGTGTTCAATTTCATG
TTCTAGTTGCTTTGTTTTACTGGTTTCACCTGTTCTATTAGGTGTTACATG
CTGTTCATCTGTTACATTGTCGATCTGTTCATGGTGAACAGCTTTAAATGC
ACCAAAAACTCGTAAAAGCTCTGATGTATCTATCTTTTTTACACCGTTTTC
ATCTGTGCATATGGACAGTTTTCCCTTTGATATCTAACGGTGAACAGTTGT
TCTACTTTTGTTTGTTAGTCTTGATGCTTCACTGATAGATACAAGAGCCAT
AAGAACCTCAGATCCTTCCGTATTTAGCCAGTATGTTCTCTAGTGTGGTTC
GTTGTTTTTGCGTGAGCCATGAGAACGAACCATTGAGATCATGCTTACTTT
GCATGTCACTCAAAAATTTTGCCTCAAAACTGGTGAGCTGAATTTTTGCAG
TTAAAGCATCGTGTAGTGTTTTTCTTAGTCCGTTACGTAGGTAGGAATCTG
ATGTAATGGTTGTTGGTATTTTGTCACCATTCATTTTTATCTGGTTGTTCT
CAAGTTCGGTTACGAGATCCATTTGTCTATCTAGTTCAACTTGGAAAATCA
ACGTATCAGTCGGGCGGCCTCGCTTATCAACCACCAATTTCATATTGCTGT
AAGTGTTTAAATCTTTACTTATTGGTTTCAAAACCCATTGGTTAAGCCTTT
TAAACTCATGGTAGTTATTTTCAAGCATTAACATGAACTTAAATTCATCAA
GGCTAATCTCTATATTTGCCTTGTGAGTTTTCTTTTGTGTTAGTTCTTTTA
ATAACCACTCATAAATCCTCATAGAGTATTTGTTTTCAAAAGACTTAACAT
GTTCCAGATTATATTTTATGAATTTTTTTAACTGGAAAAGATAAGGCAATA
TCTCTTCACTAAAAACTAATTCTAATTTTTCGCTTGAGAACTTGGCATAGT
TTGTCCACTGGAAAATCTCAAAGCCTTTAACCAAAGGATTCCTGATTTCCA
CAGTTCTCGTCATCAGCTCTCTGGTTGCTTTAGCTAATACACCATAAGCAT
TTTCCCTACTGATGTTCATCATCTGAGCGTATTGGTTATAAGTGAACGATA
CCGTCCGTTCTTTCCTTGTAGGGTTTTCAATCGTGGGGTTGAGTAGTGCCA
CACAGCATAAAATTAGCTTGGTTTCATGCTCCGTTAAGTCATAGCGACTAA
TCGCTAGTTCATTTGCTTTGAAAACAACTAATTCAGACATACATCTCAATT
GGTCTAGGTGATTTTAATCACTATACCAATTGAGATGGGCTAGTCAATGAT
AATTACTAGTCCTTTTCCTTTGAGTTGTGGGTATCTGTAAATTCTGCTAGA
CCTTTGCTGGAAAACTTGTAAATTCTGCTAGACCCTCTGTAAATTCCGCTA
GACCTTTGTGTGTTTTTTTTGTTTATATTCAAGTGGTTATAATTTATAGAA
TAAAGAAAGAATAAAAAAAGATAAAAAGAATAGATCCCAGCCCTGTGTATA
ACTCACTACTTTAGTCAGTTCCGCAGTATTACAAAAGGATGTCGCAAACGC
TGTTTGCTCCTCTACAAAACAGACCTTAAAACCCTAAAGGCTTAAGTAGCA
CCCTCGCAAGCTCGGGCAAATCGCTGAATATTCCTTTTGTCTCCGACCATC
AGGCACCTGAGTCGCTGTCTTTTTCGTGACATTCAGTTCGCTGCGCTCACG
GCTCTGGCAGTGAATGGGGGTAAATGGCACTACAGGCGCCTTTTATGGATT
CATGCAAGGAAACTACCCATAATACAAGAAAAGCCCGTCACGGGCTTCTCA
GGGCGTTTTATGGCGGGTCTGCTATGTGGTGCTATCTGACTTTTTGCTGTT
CAGCAGTTCCTGCCCTCTGATTTTCCAGTCTGACCACTTCGGATTATCCCG
TGACAGGTCATTCAGACTGGCTAATGCACCCAGTAAGGCAGCGGTATCATC
AACAGGCTTACCCGTCTTACTGTCTTTTCTACGGGGTCTGACGCTCAGTGG
AACGAAAACTCACGTTAAGGGATTTTGGTCATGAGATTATCAAAAAGGATC
TTCACCTAGATCCTTTTAAATTAAAAATGAAGTTTTAAATCAATCTAAAGT
ATATATGAGTAAACTTGGTCTGACAGTTACCAATGCTTAATCAGTGAGGCA
CCTATCTCAGCGATCTGTCTATTTCGTTCATCCATAGTTGCCTGACTCCCC
GTCGTGTAGATAACTACGATACGGGAGGGCTTACCATCTGGCCCCAGTGCT
GCAATGATACCGCGAGACCCACGCTCACCGGCTCCAGATTTATCAGCAATA
AACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTCG
CCAGTTAATAGTTTGCGCAACGTTGTTGCCATTGCTACAGGCATCGTGGTG
TCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATG
GTTATGGCAGCACTGCATAATTCTCTTACTGTCATGCCATCCGTAAGATGC
TTTTCTGTGACTGGTGAGTACTCAACCAAGTCATTCTGAGAATAGTGTATG
CGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGA
AAACTCTCAAGGATCTTACCGCTGTTGAGATCCAGTTCGATGTAACCCACT
CGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACA
CGGAAATGTTGAATACTCATACTCTTCCTTTTTCAATATTATTGAAGCATT
TATCAGGGTTATTGTCTCATGAGCGGATACATATTTGAATGTATTTAGAAA
AATAAACAAATAGGGGTTCCGCGCACATTTCCCCGAAAAGTGCCACCTGAC
GTCTAAGAAACCATTATTATCATGACATTAACCTATAAAAATAGGCGTATC
ACGAGGCCCTTTCGTCTCGCGCGTTTCGGTGATGACGGTGAAAACCTCTGA
CACATGCAGCTCCCGGAGACGGTCACAGCTTGTCTGTAAGCGGATGCCGGG
AGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATG
CGGTGTGAAATACCGCACAGATGCGTAAGGAGAAAATACCGCATCAGGCGC
CATTCGCCATTCAGGCTGCGCAACTGTTGGGAAGGGCGATCGGTGCGGGCC
TCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCC
AGTGAATTCGTTAAGACCCACTTTCACATTTAAGTTGTTTTTCTAATCCGC
ATATGATCAATTCAAGGCCGAATAAGAAGGCTGGCTCTGCACCTTGGTGAT
CAAATAATTCGATAGCTTGTCGTAATAATGGCGGCATACTATCAGTAGTAG
GTGTTTCCCTTTCTTCTTTAGCGACTTGATGCTCTTGATCTTCCAATACGC
AACCTAAAGTAAAATGCCCCACAGCGCTGAGTGCATATAATGCATTCTCTA
GTGAAAAACCTTGTTGGCATAAAAAGGCTAATTGATTTTCGAGAGTTTCAT
ACTGTTTTTCTGTAGGCCGTGTACCTAAATGTACTTTTGCTCCATCGCGAT
GACTTAGTAAAGCACATCTAAAACTTTTAGCGTTATTACGTAAAAAATCTT
GCCAGCTTTCCCCTTCTAAAGGGCAAAAGTGAGTATGGTGCCTATCTAACA
TCTCAATGGCTAAGGCGTCGAGCAAAGCCCGCTTATTTTTTACATGCCAAT
ACAATGTAGGCTGCTCTACACCTAGCTTCTGGGCGAGTTTACGGGTTGTTA
AACCTTCGATTCCGACCTCATTAAGCAGCTCTAATGCGCTGTTAATCACTT
TACTTTTATCTAATCTAGACATCATTAATTCCTAATTTTTGTTGACACTCT
ATCATTGATAGAGTTATTTT

TABLE 13
Nucleotide sequences of TetR-PheP construct, low-
copy (SEQ ID NO: 30B)

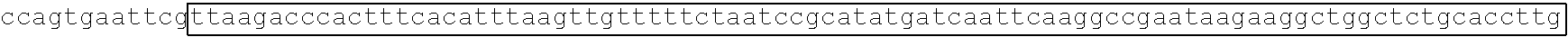

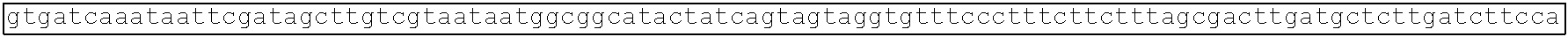

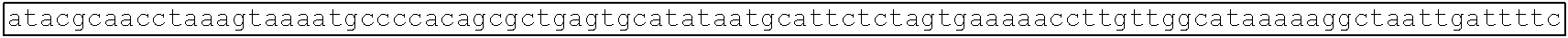

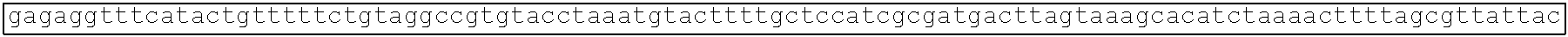

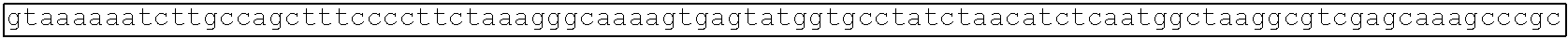

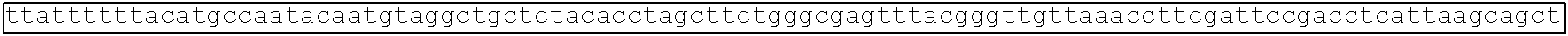

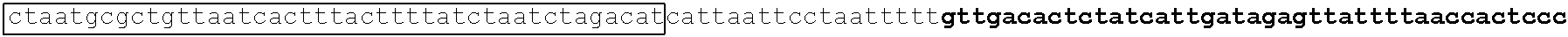

tatcagtgatagagaaaagtgaactctagaaataattttgtttaactttaagaaggagatatacatATGAAAAACGCGTCAACC
GTATCGGAAGATACTGCGTCGAATCAAGAGCCGACGCTTCATCGCGGATTACATAACCGTCATATTCAACTGA
TTGCGTTGGGTGGCGCAATTGGTACTGGTCTGTTTCTTGGCATTGGCCCGGCGATTCAGATGGCGGGTCCGGCTGTATTGCTGGG
CTACGGCGTCGCCGGGATCATCGCTTTCCTGATTATGCGCCAGCTTGGCGAAATGGTGGTTGAGG
AGCCGGTATCCGGTTCATTTGCCCACTTTGCCTATAAATACTGGGGACCGTTTGCGGGCTTCCTCTCTGGCTGGAACTACTGGG
TAATGTTCGTGCTGGTGGGAATGGCAGAGCTGACCGCTGCGGGCATCTATATGCAGTACTGGTTCC
CGGATGTTCCAACGTGGATTTGGGCTGCCGCCTTCTTTATTATCATCAACGCCGTTAACCTGGTGAACGTGCGCTTA
TATGGCGAAACCGAGTTCTGGTTTGCGTTGATTAAAGTGCTGGCAATCATCGGTATGATCGGCTTTGGCCTGT
GGCTGCTGTTTTCTGGTCACGGCGGCGAGAAAGCCAGTATCGACAACCTCTGGCGCTACGGTGGTTTCTTCGC
CACCGGCTGGAATGGGCTGATTTTGTCGCTGGCGGTAATTATGTTCTCCTTCGGCGGTCTGGAGCTGATTGGGATTA
CTGCCGCTGAAGCGCGCGATCCGGAAAAAAGCATTCCAAAAGCGGTAAATCAGGTGGTGTATCGCATCCTGCTGTTTTACAT
CGGTTCACTGGTGGTTTTACTGGCGCTCTATCCGTGGGTGGAAGTGAAATCCAACAGTAGCCCGTTTG
TGATGATTTTCCATAATCTCGACAGCAACGTGGTAGCTTCTGCGCTGAACTTCGTCATTCTGGTAGCATCGCTGTCAGTG
TATAACAGCGGGGTTTACTCTAACAGCCGCATGCTGTTTGGCCTTTCTGTGCAGGGTAATGCGCCGAAGT
TTTTGACTCGCGTCAGCCGTCGCGGTGTGCCGATTAACTCGCTGATGCTTTCCGGAGCGATCACTTCGCTGGTGGTGTTAATC
AACTATCTGCTGCCGCAAAAAGCGTTTGGTCTGCTGATGGCGCTGGTGGTAGCAACGCTGCTGTTGA
ACTGGATTATGATCTGTCTGGCGCATCTGCGTTTTCGTGCAGCGATGCGACGTCAGGGGCGTGAAACACAGTTTA
AGGCGCTGCTCTATCCGTTCGGCAACTATCTCTGCATTGCCTTCCTCGGCATGATTTTGCTGCTGATGTGCACGA
TGGATGATATGCGCTTGTCAGCGATCCTGCTGCCGGTGTGGATTGTATTCCTGTTTATGGCATTTAAAACGCTGCGTCGGAAATAA

In some embodiments, the genetically engineered bacteria contain gene sequence(s) comprising one or more sequence(s) of any of SEQ ID Nos: 21B-30B. In some embodiments, the genetically engineered bacteria contain gene sequence(s) comprising one or more sequence(s) having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID Nos: 21B-30B.

In any of the embodiments described herein, the genetically engineered bacteria comprising one or more genes encoding one or more PME(s) further comprise one or more endogenous bacteriophage genomes. In some embodiments, the bacteriophage has been mutated in one or more genes within the bacteriophage genome. Such mutations include deletions, insertions, substitutions and inversions and may be located in or encompass one or more bacteriophage genes.

In one embodiment, E. coli Nissle is used as a starting point, parental strain or “chassis” for the genetically engineered bacteria comprising one or more PME(s). In one embodiment, the bacteriophage which is modified is a phage which is endogenous to E. coli Nissle in its natural state.

In some embodiments, the genetically engineered bacteria comprise one or more E. coli. Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In some of the preceding embodiments, the mutation is a deletion. In some embodiments, the genetically engineered bacteria comprise one or more deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one embodiment, the genetically engineered bacteria comprise a complete or partial deletion of one or more ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Oxygen Consuming Enzymes

LAAD catalytic activity is dependent on oxygen, and therefore may not be active in anaerobic and/or low oxygen environments in the intestine, e.g., the colon. Oxygen is present in more proximal compartments of the GI tract.

The oxygen tension as measured in healthy mice is shown in Table 17A. He et al., Proc Natl Acad Sci USA. 1999 Apr. 13; 96(8):4586-91; “Noninvasive measurement of anatomic structure and intraluminal oxygenation in the gastrointestinal tract of living mice with spatial and spectral EPR imaging”, the contents of which is herein incorporated by reference in its entirety. A marked oxygen gradient from the proximal to the distal GI tract. As noted by He, the observed oxygen gradient seen along the GI tract can be explained by a combination of processes. Without wishing to be bound by theory, food, when swallowed, is initially equilibrated with the oxygen tension of ambient room air. On passage to the stomach and later the small intestine, the oxygen levels may fall as oxygen diffuses across the mucosal membrane. A gradual process of equilibration with the capillary levels of oxygen (i.e., 5-10 torr; ref. 9) may occur. On passage to the colon, with its heavy bacterial colonization, further decreases in oxygenation occur. Finally, the lumen of the distal colon displays marked hypoxia, as expected, based on the abundance of anaerobic bacteria at this site.

TABLE 17A
Oxygen Tension in Gastrointestinal Tract Compartments

| Compartment | Oxygen Tension |
|---|---|
| Ambient Air | 159 Torr |
| stomach | ~60 torr |
| duodenum and first | (~30 torr); ~20% oxygen in ambient air |
| part of jejunum | |
| Ileum | (~10 torr); ~6% oxygen in ambient air |
| colon | (<2 torr) |

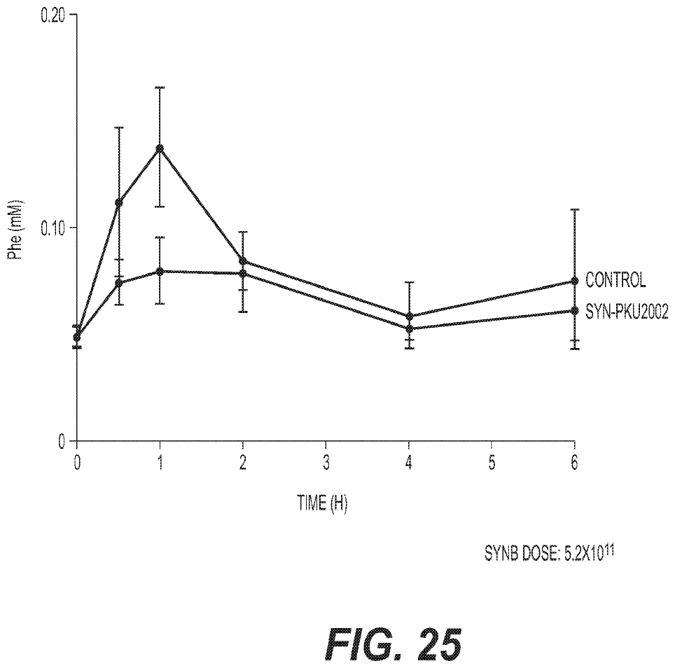

As shown in FIG. 25B, LAAD activity is retained in microaerobic conditions, albeit at lower levels than under aerobic conditions (FIG. 25A and FIG. 25B). LAAD therefore may be active in the more proximal areas of the intestine, such as stomach, duodenum, jejunum, and ileum. It is contemplated as part of this disclosure that LAAD expressed by the genetically engineered bacteria may advantageously be active in a different compartment than PAL, which may be expressed in the colon if under the control of an FNR promoter. In one embodiment, the genetically engineered bacteria express two enzymes, which have different oxygen requirements and/or are induced under different oxygen conditions, such that an PME is expressed and active throughout the entire gastrointestinal system. For example, the first enzyme, e.g., LAAD, which is dependent on the presence of oxygen, is expressed in one or more of stomach, duodenum and ileum under the control of a constitutive or inducible promoter (such as ParaBAD), and the second enzyme, e.g., PAL, is expressed in the colon under the control of an FNR promoter. In some embodiments, PAL is expressed under the conditions found in the small intestine, e.g. under the control of an FNR promoter, constitutive promoter, or a different inducible promoter described herein. In some embodiments, PAL and/or LAAD are pre-induced prior to in vivo administration, and are expressed and active in the proximal part of the intestine. In some embodiments, PAL and/or LAAD are pre-induced (aerobically or anaerobically, or with or without chemical and/or nutritional inducer, as described herein) prior to in vivo administration, and are expressed and active in the distal part of the intestine.

Several strategies can be employed to further increase LAAD activity under oxygen limiting conditions. For example, the activity of other enzymes that consume large amounts of oxygen can be reduced or extinguished. One such enzyme is NADH dehydrogenase. E. coli has two NADH dehydrogenases; nuo and ndh2, and is has been shown that knock out of both of these enzymes reduces oxygen consumption by 80%. In some embodiments, additional measures are taken to conserve limiting oxygen, i.e., to allow LAAD to function under lower exogenous oxygen conditions in the genetically engineered bacteria expressing LAAD. In some embodiments, the genetically engineered bacteria further comprise a mutation in one or more genes involved in oxygen consumption. In some embodiments, one or both E. coli NADH dehydrogenases are knocked out. In some embodiments, the knocked out NADH dehydrogenase is nuo. In some embodiments, the knocked out NADH dehydrogenase is ndh2. In some embodiments nuo and ndh2 are knocked out. Other enzymes involved in E. coli oxygen metabolism may also be knocked out, including enzymes in the respiratory chain, such as cydB (a subunit of high affinity terminal oxidase), cydD (an enzyme required to make cytochrome D), and cyoABC (subunits of low affinity cytochrome oxidase). In some embodiments, the genetically engineered bacteria harbor a knock out mutation/deletion in one more genes selected from cydB, cydD, and cyoABC.

In one embodiment, the one or more PME encoded by the genetically engineered bacteria are expressed and show activity in the stomach. In one embodiment, the one or more PME encoded by the genetically engineered bacteria are expressed and show activity in the duodenum. In one embodiment, the one or more PME encoded by the genetically engineered bacteria are expressed and show activity in the jejunum. In one embodiment, the one or more PME encoded by the genetically engineered bacteria are expressed and show activity in the ileum. In one embodiment, the one or more PME encoded by the genetically engineered bacteria are expressed and show activity in the colon.

### Phenylalanine Transport

In some embodiments wherein the bacterium comprises a gene encoding a PME, the bacterium may further comprise a gene encoding a phenylalanine transporter. Phenylalanine transporters may be expressed or modified in the genetically engineered bacteria of the invention in order to enhance phenylalanine transport into the cell.

PheP is a membrane transport protein that is capable of transporting phenylalanine into bacterial cells (see, e.g., Pi et al., 1991). In some embodiments, the native pheP gene in the genetically modified bacteria of the invention is not modified. In some embodiments, the genetically engineered bacteria of the invention comprise multiple copies of the native pheP gene. In some embodiments, the genetically engineered bacteria of the invention comprise multiple copies of a non-native pheP gene. In some embodiments, the genetically engineered bacteria of the invention comprise a pheP gene that is controlled by its native promoter, an inducible promoter, a promoter that is stronger than the native promoter, e.g., the GlnRS promoter or the P(Bla) promoter, or a constitutive promoter. In some embodiments, expression of the pheP gene is controlled by a different promoter than the promoter that controls expression of the gene encoding the phenylalanine-metabolizing enzyme and/or the transcriptional regulator. In some embodiments, expression of the pheP gene is controlled by the same promoter that controls expression of the phenylalanine-metabolizing enzyme and/or the transcriptional regulator. In some embodiments, the pheP gene and the phenylalanine-metabolizing enzyme and/or the transcriptional regulator are divergently transcribed from a promoter region. In some embodiments, expression of each of the genes encoding PheP, the phenylalanine-metabolizing enzyme, and the transcriptional regulator is controlled by a different promoter. In some embodiments, expression of the genes encoding PheP, the phenylalanine-metabolizing enzyme, and the transcriptional regulator is controlled by the same promoter.

In some embodiments, the native pheP gene in the genetically modified bacteria is not modified, and one or more additional copies of the native pheP gene are inserted into the genome under the control of the same inducible promoter that controls expression of PAL, e.g., the FNR promoter, or a different inducible promoter than the one that controls expression of PAL, or a constitutive promoter. In alternate embodiments, the native pheP gene is not modified, and a copy of a non-native pheP gene from a different bacterial species is inserted into the genome under the control of the same inducible promoter that controls expression of PAL, e.g., the FNR promoter, or a different inducible promoter than the one that controls expression of PAL, or a constitutive promoter.

In some embodiments, the native pheP gene in the genetically modified bacteria is not modified, and one or more additional copies of the native pheP gene are present in the bacteria on a plasmid and under the control of the same inducible promoter that controls expression of PAL, e.g., the FNR promoter, or a different inducible promoter than the one that controls expression of the PME, or a constitutive promoter. In alternate embodiments, the native pheP gene is not modified, and a copy of a non-native pheP gene from a different bacterial species is present in the bacteria on a plasmid and under the control of the same inducible promoter that controls expression of PAL, e.g., the FNR promoter, or a different inducible promoter than the one that controls expression of PAL, or a constitutive promoter.

In some embodiments, the native pheP gene is mutagenized, mutants exhibiting increased phenylalanine transport are selected, and the mutagenized pheP. The phenylalanine transporter modifications described herein may be present on a plasmid or chromosome.

In some embodiments, the genetically engineered bacterium is E. coli Nissle, and the native pheP gene in E. coli Nissle is not modified; one or more additional copies the native E. coli Nissle pheP genes are inserted into the E. coli Nissle genome under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium is inserted into the E. coli Nissle genome under the control of the same inducible promoter that controls expression of PAL, e.g., the FNR promoter, or a different inducible promoter than the one that controls expression of PAL, or a constitutive promoter. In some embodiments, the genetically engineered bacterium is E. coli Nissle, and the native pheP gene in E. coli Nissle is not modified; one or more additional copies the native E. coli Nissle pheP genes are present in the bacterium on a plasmid and under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium, are present in the bacterium on a plasmid and under the control of the same inducible promoter that controls expression of PAL, e.g., the FNR promoter, or a different inducible promoter than the one that controls expression of PAL, or a constitutive promoter.

In other embodiments, the gene(s) encoding the one or more Phe transporter(s) may be located on a plasmid or in the chromosome and the gene expression may be regulated by any of the promoters disclosed herein, which may be the same or different from the promoters regulating the PME gene(s).

It has been reported that Escherichia coli has five distinct transport systems (AroP, Mtr, PheP, TnaB, and TyrP) for the accumulation of aromatic amino acids. A general amino acid permease, encoded by the aroP gene, transports three aromatic amino acids, including phenylalanine, with high affinity, and is thought, together with PheP, responsible for the lion share of phenylalanine import. Additionally, a low level of accumulation of phenylalanine was observed in an aromatic amino acid transporter-deficient E. coli strain (AaroP ApheP Amtr Atna AtyrP), and was traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF (Koyanagi et al., and references therein; Identification of the LIV-I/LS System as the Third Phenylalanine Transporter in Escherichia coli K-12).

In some embodiments, the genetically engineered bacteria comprise an aroP gene. In some embodiments, the genetically engineered bacterium is E. coli Nissle, and the native aroP gene in E. coli Nissle is not modified; one or more additional copies of the native E. coli Nissle aroP genes are present in the bacterium on a plasmid or in the chromosome and under the control of the same inducible promoter that controls expression of the PME, e.g., the FNR promoter, or the araBAD promoter, a different inducible promoter than the one that controls expression of the PME, or a constitutive promoter. In an alternate embodiment, the native aroP gene in E. coli Nissle is not modified, and a copy of a non-native aroP gene from a different bacterium, are present in the bacterium on a plasmid or in the chromosome and under the control of the same inducible promoter that controls expression of the PME, e.g., the FNR promoter or the AraBAD promoter, or a different inducible promoter than the one that controls expression of the PME, or a constitutive promoter.

In other embodiments, the genetically engineered bacteria comprise AroP and PheP, under the control of the same or different inducible or constitutive promoters.

In some embodiments, the pheP gene is expressed on a chromosome. In some embodiments, expression from the chromosome may be useful for increasing stability of expression of pheP. In some embodiments, the pheP gene is integrated into the bacterial chromosome at one or more integration sites in the genetically engineered bacteria. In some embodiments, the pheP gene is inserted into the bacterial genome at one or more of the following insertion sites in E. coli Nissle: malE/K, insB/I, araC/BAD, lacZ, agal/rsml, thyA, and malP/T. Any suitable insertion site may be used (see, e.g., FIG. 66 of WO2017087580, the contents of which are herein incorporated by reference in their entirety). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon.

In some embodiments, the genetically engineered bacteria encode one or more Phe transporter(s) which are directly or indirectly pre-induced prior to in vivo administration, e.g., are expressed under the control of an inducible promoter that is responsive to specific molecules or metabolites in the provided in the culture of the bacterium in a flask, fermenter, or other culture vessel, during production of the strain prior to in vivo administration.

In other embodiments, the genetically engineered bacteria encode one or more Phe transporter(s) which are directly or indirectly induced in vivo administration, e.g., are expressed under the control of an inducible promoter that is responsive conditions or to specific molecules or metabolites in the exogenous in vivo environment, e.g., the gut. In some embodiments, the promoter is induced by gut specific molecules, or low oxygen conditions. In some embodiments, the bacterial strains are administered in combination with a chemical and/or nutritional inducer.

In some embodiments, the genetically engineered bacterium comprises multiple mechanisms of action and/or one or more auxotrophies. In certain embodiments, the bacteria are genetically engineered to comprise five copies of PAL under the control of an oxygen level-dependent promoter (e.g., P_{fnrS}-PAL3) inserted at different integration sites on the chromosome (e.g., malE/K, yicS/nepI, malP/T, agaI/rsmI, and cea), and one copy of a phenylalanine transporter gene under the control of an oxygen level-dependent promoter (e.g., P_{fnrS}-pheP) inserted at a different integration site on the chromosome (e.g., lacZ). In a more specific aspect, the bacteria are genetically engineered to further include a kanamycin resistance gene, and a thyA auxotrophy, in which the thyA gene is deleted and/or replaced with an unrelated gene.

In any of the embodiments described herein, the genetically engineered bacteria comprising one or more genes encoding one or more phenylalanine transporters further comprise one or more endogenous bacteriophages. In some embodiments, the bacteriophage(s) have been mutated in one or more genes within the bacteriophage genome. Such mutations include deletions, insertions, substitutions and inversions and are located in or encompass one or more bacteriophage genes.

In some embodiments, expression of the pheP gene is controlled by a different promoter than the promoter that controls expression of the gene encoding the phenylalanine-metabolizing enzyme and/or the transcriptional regulator. In some embodiments, expression of the pheP gene is controlled by the same promoter that controls expression of the phenylalanine-metabolizing enzyme and/or the transcriptional regulator. In some embodiments, the pheP gene and the phenylalanine-metabolizing enzyme and/or the transcriptional regulator are divergently transcribed from a promoter region. In some embodiments, expression of each of the genes encoding PheP, the phenylalanine-metabolizing enzyme, and the transcriptional regulator is controlled by a different promoter. In some embodiments, expression of the genes encoding PheP, the phenylalanine-metabolizing enzyme, and the transcriptional regulator is controlled by the same promoter.

In some embodiments, the native pheP gene in the genetically modified bacteria is not modified, and one or more additional copies of the native pheP gene are inserted into the genome under the control of the same inducible promoter that controls expression of PAL, e.g., the FNR promoter, or a different inducible promoter than the one that controls expression of PAL, or a constitutive promoter. In alternate embodiments, the native pheP gene is not modified, and a copy of a non-native pheP gene from a different bacterial species is inserted into the genome under the control of the same inducible promoter that controls expression of PAL, e.g., the FNR promoter, or a different inducible promoter than the one that controls expression of PAL, or a constitutive promoter.

In some embodiments, the native pheP gene in the genetically modified bacteria is not modified, and one or more additional copies of the native pheP gene are present in the bacteria on a plasmid and under the control of the same inducible promoter that controls expression of PAL, e.g., the FNR promoter, or a different inducible promoter than the one that controls expression of the PME, or a constitutive promoter. In alternate embodiments, the native pheP gene is not modified, and a copy of a non-native pheP gene from a different bacterial species is present in the bacteria on a plasmid and under the control of the same inducible promoter that controls expression of PAL, e.g., the FNR promoter, or a different inducible promoter than the one that controls expression of PAL, or a constitutive promoter.

In some embodiments, the native pheP gene is mutagenized, mutants exhibiting increased phenylalanine transport are selected, and the mutagenized pheP The phenylalanine transporter modifications described herein may be present on a plasmid or chromosome.

In some embodiments, the genetically engineered bacterium is E. coli Nissle, and the native pheP gene in E. coli Nissle is not modified; one or more additional copies the native E. coli Nissle pheP genes are inserted into the E. coli Nissle genome under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium is inserted into the E. coli Nissle genome under the control of the same inducible promoter that controls expression of PAL, e.g., the FNR promoter, or a different inducible promoter than the one that controls expression of PAL, or a constitutive promoter. In some embodiments, the genetically engineered bacterium is E. coli Nissle, and the native pheP gene in E. coli Nissle is not modified; one or more additional copies the native E. coli Nissle pheP genes are present in the bacterium on a plasmid and under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium, are present in the bacterium on a plasmid and under the control of the same inducible promoter that controls expression of PAL, e.g., the FNR promoter, or a different inducible promoter than the one that controls expression of PAL, or a constitutive promoter.

In other embodiments, the gene(s) encoding the one or more Phe transporter(s) may be located on a plasmid or in the chromosome and the gene expression may be regulated by any of the promoters disclosed herein, which may be the same or different from the promoters regulating the PME gene(s).

It has been reported that Escherichia coli has five distinct transport systems (AroP, Mtr, PheP, TnaB, and TyrP) for the accumulation of aromatic amino acids. A general amino acid permease, encoded by the aroP gene, transports three aromatic amino acids, including phenylalanine, with high affinity, and is thought, together with PheP, responsible for the lion share of phenylalanine import. Additionally, a low level of accumulation of phenylalanine was observed in an aromatic amino acid transporter-deficient E. coli strain (ΔaroP ΔpheP Δmtr Δtna ΔtyrP), and was traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF (Koyanagi et al., and references therein; Identification of the LIV-I/LS System as the Third Phenylalanine Transporter in Escherichia coli K-12).

In some embodiments, the genetically engineered bacteria comprise an aroP gene. In some embodiments, the genetically engineered bacterium is E. coli Nissle, and the native aroP gene in E. coli Nissle is not modified; one or more additional copies of the native E. coli Nissle aroP genes are present in the bacterium on a plasmid or in the chromosome and under the control of the same inducible promoter that controls expression of the PME, e.g., the FNR promoter, or the araBAD promoter, a different inducible promoter than the one that controls expression of the PME, or a constitutive promoter. In an alternate embodiment, the native aroP gene in E. coli Nissle is not modified, and a copy of a non-native aroP gene from a different bacterium, are present in the bacterium on a plasmid or in the chromosome and under the control of the same inducible promoter that controls expression of the PME, e.g., the FNR promoter or the AraBAD promoter, or a different inducible promoter than the one that controls expression of the PME, or a constitutive promoter.

In other embodiments, the genetically engineered bacteria comprise AroP and PheP, under the control of the same or different inducible or constitutive promoters.

In some embodiments, the pheP gene is expressed on a chromosome. In some embodiments, expression from the chromosome may be useful for increasing stability of expression of pheP. In some embodiments, the pheP gene is integrated into the bacterial chromosome at one or more integration sites in the genetically engineered bacteria. In some embodiments, the pheP gene is inserted into the bacterial genome at one or more of the following insertion sites in E. coli Nissle: malE/K, insB/I, araC/BAD, lacZ, agal/rsml, thyA, and malP/T. Any suitable insertion site may be used (see, e.g., FIG. 66 of WO2017087580, the contents of which are herein incorporated by reference in their entirety). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon.

In some embodiments, the genetically engineered bacteria encode one or more Phe transporter(s) which are directly or indirectly pre-induced prior to in vivo administration, e.g., are expressed under the control of an inducible promoter that is responsive to specific molecules or metabolites in the provided in the culture of the bacterium in a flask, fermenter, or other culture vessel, during production of the strain prior to in vivo administration.

In other embodiments, the genetically engineered bacteria encode one or more Phe transporter(s) which are directly or indirectly induced in vivo administration, e.g., are expressed under the control of an inducible promoter that is responsive conditions or to specific molecules or metabolites in the exogenous in vivo environment, e.g., the gut. In some embodiments, the promoter is induced by gut specific molecules, or low oxygen conditions. In some embodiments, the bacterial strains are administered in combination with a chemical and/or nutritional inducer.

In some embodiments, the genetically engineered bacterium comprises multiple mechanisms of action and/or one or more auxotrophies. In certain embodiments, the bacteria are genetically engineered to comprise five copies of PAL under the control of an oxygen level-dependent promoter (e.g., P_{fnrS}-PAL3) inserted at different integration sites on the chromosome (e.g., malE/K, yicS/nepI, malP/T, agaI/rsmI, and cea), and one copy of a phenylalanine transporter gene under the control of an oxygen level-dependent promoter (e.g., P_{fnrS}-pheP) inserted at a different integration site on the chromosome (e.g., lacZ). In a more specific aspect, the bacteria are genetically engineered to further include a kanamycin resistance gene, and a thyA auxotrophy, in which the thyA gene is deleted and/or replaced with an unrelated gene.

In any of the embodiments described herein, the genetically engineered bacteria comprising one or more genes encoding one or more phenylalanine transporters further comprise one or more endogenous bacteriophages. In some embodiments, the bacteriophage(s) have been mutated in one or more genes within the bacteriophage genome. Such mutations include deletions, insertions, substitutions and inversions and are located in or encompass one or more bacteriophage genes.

In one embodiment, E. coli Nissle is used as a starting point, parental strain or “chassis” for the genetically engineered bacteria. In one embodiment, the bacteriophage which is modified is a phage which is endogenous to E. coli Nissle in its natural state.

In some embodiments, the genetically engineered bacteria comprise one or more E. coli Nissle bacteriophage, e.g., Phage 1, Phage2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In some embodiments, the mutation is a deletion. In some embodiments, the genetically engineered bacteria comprise one or more deletions

### Multiple Mechanisms of Action

In some embodiments of the disclosure, modifications to the genome of EcN have been made to enhance phenylalanine degradation under the low oxygen conditions found in the gut while augmenting biologic containment through diaminopimelate auxotrophy. These genetically engineered bacteria further comprise one or more modifications to endogenous EcN phage 3.

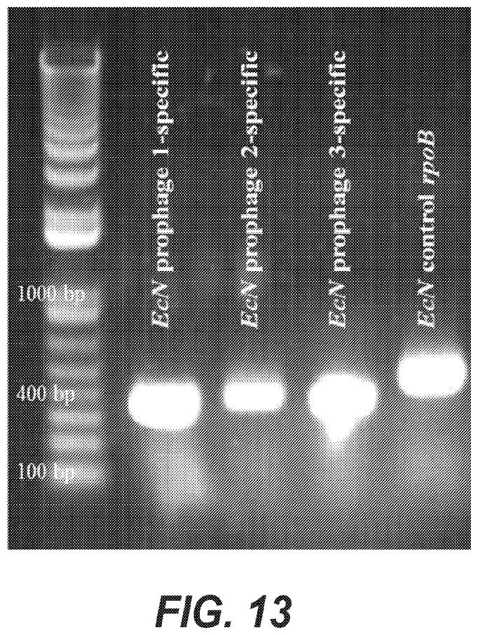

In some embodiments, the bacteria are genetically engineered to include multiple mechanisms of action (MoAs), e.g., circuits producing multiple copies of the same product (e.g., to enhance copy number) or circuits performing multiple different functions. Examples of insertion sites include, but are not limited to, malE/K, yicS/nepI, insB/I, araC/BAD, lacZ, agal/rsml, thyA, malP/T, dapA, and cea, and others shown in FIG. 66 of WO2017087580, the contents of which are herein incorporated by reference in their entirety. For example, the genetically engineered bacteria may include four copies of a payload inserted at four different insertion sites, e.g., malE/K, insB/I, araC/BAD, and lacZ. The genetically engineered bacteria may also include four copies of the same or different payload inserted at four different insertion sites, e.g., malE/K, yicS/nepI, agaI/rsmI, and cea, and one copy a third same or different gene inserted at a different insertion site, e.g., lacZ (FIG. 13B of WO2017087580, the contents of which are herein incorporated by reference in their entirety). Alternatively, the genetically engineered bacteria may include three copies of a payload inserted at three different insertion sites, e.g., malE/K, insB/I, and lacZ.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut or the tumor microenvironment, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

In some embodiments, under conditions where the gene sequence(s) for producing the payload(s), are expressed, the bacterium produces an effector or metabolizes a substrate at levels at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold greater as compared to unmodified bacteria of the same subtype under the same conditions.

In any of these embodiments, the genetically engineered bacteria further contain one or more mutations or modifications to an endogenous phage genome. In some embodiments, the mutations are deletion, insertion, substitution or inversions within the phage genome. IN some embodiments, the mutations are deletions. In some embodiments, the deletions comprise one or more phage genes. In some embodiments, phage genes are partially deleted. In some embodiments, the mutations are insertions. In some embodiments, the insertion comprises an antibiotic cassette as described herein. IN some embodiments, one or more genes are substituted. In some embodiments, the substitution comprises an antibiotic cassette. In some embodiments, one or more phage genes are inverted. In some embodiments parts or one or more phage genes are inverted.

In one embodiment, the E. coli Nissle bacteria described herein comprise one or more modifications or mutations, e.g., deletion, insertion, substitution or inversion, within the E. coli Nissle Phage 3 genome. In some embodiments, the mutation is an insertion. In some embodiments, the insertion comprises an antibiotic cassette as described herein. In some embodiments, the mutation is a deletion. In any of the embodiments described herein, the deletions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

Induction of PMEs and/or Phe Transporters During Strain Culture

For induction and preinduction of PME's and/or Phe Transporters described herein, protocols and strategies were employed as described in PCT/WO2017/087580 A1, the entire contents of which are expressly incorporated herein by reference in its entirety.

In any of the embodiments described herein, the genetically engineered bacteria comprise one or more PMEs and/or one or more transporters which are induced under manufacturing conditions (e.g., aerobic, anaerobic, low-oxygen, or microaerobic) comprise one or more endogenous bacteriophage genomes. In some embodiments, the bacteriophage(s) have been mutated in one or more genes within the bacteriophage genome. Such mutations include deletions, insertions, substitutions and inversions and are located in or encompass one or more bacteriophage genes.

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Measurement of Pre-Induction

In some embodiments, such culture conditions, in which expression of the PME(s) and or PheP are induced result in the reduction of phenylalanine in the culture by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions, or as compared to the baseline levels. In some embodiments, such culture conditions, in which expression of the PME(s) and or PheP are induced result in the production of transcinnamic acid (TCA) in the culture by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions, or as compared to the baseline levels.

In some embodiments, cinnamate accumulation in the bacterial cultures is measured by methods known in the art and described herein. Cinnamate production is directly correlated with phenylalanine degradation, and in some embodiments, cinnamate may be used as an indicator for strain activity during strain growth, production and manufacture. Measurement of a reduction in phenylalanine and or the production of TCA therefore may be used to measure and monitor, and fine tune the induction of a therapeutic strain prior to administration in vivo.

### Phage 3

PAL inserted at four different insertion sites, e.g., malE/K, insB/I, araC/BAD, and lacZ. The genetically engineered bacteria may also include four copies of PAL inserted at four different insertion sites, e.g., malE/K, yicS/nepI, agaI/rsmI, and cea, and one copy of a phenylalanine transporter gene inserted at a different insertion site, e.g., lacZ (FIG. 13B). Alternatively, the genetically engineered bacteria may include three copies of PAL inserted at three different insertion sites, e.g., malE/K, insB/I, and lacZ, and three copies of a phenylalanine transporter gene inserted at three different insertion sites, e.g., dapA, cea, and araC/BAD. In any of these embodiments, the genetically engineered bacteria further contain one or more mutations or modifications to EcN endogenous Phage 3.

In some embodiments, the genetically engineered bacteria comprise one or more gene sequence(s) encoding one or more of (1) PAL, PAH, and/or LAAD for degradation of phenylalanine, in wild type or in a mutated form (for increased stability or metabolic activity) (2) transporter PheP and/or AroP for uptake of phenylalanine, in wild type or in mutated form (for increased stability or metabolic activity) (3) PAL, PAH, LAAD, and/or PheP for secretion and extracellular phenylalanine degradation, (4) components of secretion machinery, as described herein (5) Auxotrophy, e.g., deltaThyA and/or deltadapA (6) antibiotic resistance, including but not limited to, kanamycin or chloramphenicol resistance (7) mutations/deletions in genes involved in oxygen metabolism, as described herein and (8) mutations/deletions in genes of the endogenous Nissle phenylalanine synthesis pathway (e.g., delta PheA for Phe auxotrophy) (9) one or more biosafety systems constructs and/or kill switches (10) one or more other regulatory factors, e.g., FNRS24Y (11) one or more modifications or mutations, e.g., deletion, insertion, substitution or inversion, within the E. coli Nissle Phage 3 genome.

In any of the embodiments described herein, the genetically engineered bacteria for the consumption of phenylalanine further comprise one or more endogenous bacteriophage genomes. In some embodiments, the bacteriophage(s) have been mutated in one or more genes within the bacteriophage genome. Such mutations include deletions, insertions, substitutions and inversions and are located in or encompass one or more bacteriophage genes.

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In some embodiments, the mutation is a deletion. In some embodiments, the genetically engineered bacteria comprise one or more deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, under conditions where the gene sequence(s) for producing the payload(s), e.g., the PME(s), Phe tranporter(s), and/or transcriptional regulator(s) are expressed, the genetically engineered bacteria of the disclosure both degrade phenylalanine and generate TCA at levels at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold greater as compared to unmodified bacteria of the same subtype under the same conditions.

In some embodiments, under conditions where the gene sequence(s) for producing the payload(s), e.g., the PME(s), Phe tranporter(s), and/or transcriptional regulator(s) are expressed, the genetically engineered bacteria of the disclosure both degrade phenylalanine and generate hippurate at levels at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold greater as compared to unmodified bacteria of the same subtype under the same conditions.

In some embodiments, the gene sequence(s) encoding the PME(s) and/or Phe transporters, e.g., PheP, and/or other regulatory proteins, e.g., FNRS24Y_are expressed under the control of a constitutive promoter. In another embodiment, the gene sequence(s) encoding the one or more PME(s) and/or Phe transporters, e.g., PheP, and/or other regulatory proteins, e.g., FNRS24Y are expressed under the control of an inducible promoter. In some embodiments, the gene sequence(s) encoding the one or more PME(s) and/or Phe transporters, e.g., PheP, and/or other regulatory proteins, e.g., FNRS24Y, are expressed under the control of a promoter that is directly or indirectly induced by exogenous environmental conditions. In one embodiment, the gene sequence(s) encoding the one or more PME(s) and/or Phe transporters, e.g., PheP, and/or other regulatory proteins, e.g., FNRS24Y, are expressed under the control of a promoter that is directly or indirectly induced by low-oxygen or anaerobic conditions, wherein expression of the gene sequence(s) encoding the PME(s) and/or Phe transporters, e.g., PheP, and/or other regulatory proteins, e.g., FNRS24Y, are activated under low-oxygen or anaerobic environments, such as the environment of the mammalian gut. Exemplary inducible promoters described herein include oxygen level-dependent promoters (e.g., FNR-inducible promoter) arabinose, tetracycline, IPTG, rhamnose, and other chemical and/or nutritional inducers. In some embodiments, such inducible promoters described herein, are induced under in vitro culture conditions, as strains are prepared prior to in vivo administration, as described herein. Examples of inducible promoters include, but are not limited to, an FNR responsive promoter, a P_{araBAD} promoter, and a P_{TetR} promoter, Plac promoter, the rhaP BAD (rhamnose) promoter, each of which are described in more detail herein. Inducible promoters are described in more detail infra.

The at least one gene encoding the one or more PME(s) and/or Phe transporters, e.g., PheP, and/or other regulatory proteins, e.g., FNRS24Y, may be present on a plasmid or chromosome in the bacterial cell. In one embodiment, the gene sequence(s) encoding the one or more PME(s) and/or Phe transporters, e.g., PheP, and/or other regulatory proteins, e.g., FNRS24Y, are located on a plasmid in the bacterial cell. In another embodiment, the gene sequence(s) encoding the one or more PME(s) and/or Phe transporters, e.g., PheP, and/or other regulatory proteins, e.g., FNRS24Y, are located in the chromosome of the bacterial cell. In yet another embodiment, a native copy of the gene sequence(s) encoding the one or more PME(s) and/or Phe transporters, e.g., PheP, and/or other regulatory proteins, e.g., FNRS24Y, are located in the chromosome of the bacterial cell, and one or more gene(s) encoding one or more PME(s) and/or Phe transporters, e.g., PheP, and/or other regulatory proteins, e.g., FNRS24Y, are located on a plasmid in the bacterial cell. In yet another embodiment, a native copy of the gene sequence(s) encoding the one or more PME(s) and/or Phe transporters, e.g., PheP, and/or other regulatory proteins, e.g., FNRS24Y, are located on a plasmid in the bacterial cell, and at least one gene encoding the at least one or more PME(s) and/or Phe transporters, e.g., PheP, and/or other regulatory proteins, e.g., FNRS24Y, from a different species of bacteria are located on a plasmid in the bacterial cell. In yet another embodiment, a native copy of the gene sequence(s) encoding the one or more PME(s) and/or Phe transporters, e.g., PheP, and/or other regulatory proteins, e.g., FNRS24Y, are located in the chromosome of the bacterial cell, and the one or more gene(s) encoding the one or more PME(s) and/or Phe transporters, e.g., PheP, and/or other regulatory proteins, e.g., FNRS24Y, from a different species of bacteria are located in the chromosome of the bacterial cell. In some embodiments, the gene sequence(s) encoding the one or more PME(s) and/or Phe transporters, e.g., PheP, and/or other regulatory proteins, e.g., FNRS24Y, are expressed on a low-copy plasmid. In some embodiments, the gene sequence(s) encoding the one or more PME(s) and/or Phe transporters, e.g., PheP, and/or other regulatory proteins, e.g., FNRS24Y, are expressed on a high-copy plasmid. In some embodiments, the high-copy plasmid may be useful for increasing expression of the at least one PME(s) and/or Phe transporters, e.g., PheP, and/or other regulatory proteins, e.g., FNRS24Y. In some embodiments, the genetically engineered bacteria described above further comprise one or more of the modifications, mutations, and/or deletions in endogenous genes described herein. In any of the embodiments described herein, the genetically engineered bacteria comprise one or more endogenous bacteriophage genomes. In some embodiments, the bacteriophage(s) have been mutated in one or more genes within the bacteriophage genome. Such mutations include deletions, insertions, substitutions and inversions and may be located in or encompass one or more bacteriophage genes.

In some embodiments, the genetically engineered bacteria comprise one or more E. coli Nissle bacteriophage, e.g., Phage 1, Phage2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In some embodiments, the mutation is a deletion. In some embodiments, the genetically engineered bacteria comprise one or more deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In one embodiment, the genetically engineered bacterial strain comprises three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of the mutated FNR transcription factor FNRS24Y The strain further comprises one copy of LAAD knocked into the arabinose operon with expression driven by the native Para promoter (Para::FNRS24Y, e.g., SEQ ID NO: 64 of WO2017087580, the contents of which are herein incorporated by reference in their entirety). In one embodiment, the genetically engineered bacterial strain is SYN-PKU707.

In one embodiment, the genetically engineered bacterial strain comprises three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of the mutated FNR transcription factor FNRS24Y knocked into the arabinose operon with expression driven by the native Para promoter (Para::FNRS24Y e.g., SEQ ID NO: 64 of WO2017087580, the contents of which are herein incorporated by reference in their entirety). The genome is further engineered to include a dapA auxotrophy, in which the dapA gene is deleted. In one embodiment, the genetically engineered bacterial strain is SYN-PKU712.

In one embodiment, the genetically engineered bacterial strain comprises a bacterial chromosome with three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of the mutated FNR transcription factor FNRS24Y knocked into the arabinose operon with expression driven by the native Para promoter and one copy of LAAD inserted at the same insertion site (Para::FNRS24Y-LAAD, e.g., SEQ ID NO: 73 of WO2017087580, the contents of which are herein incorporated by reference in their entirety), which is transcribed as a bicistronic message from the endogenous arabinose promoter. The genome is further engineered to include a dapA auxotrophy, in which the dapA gene is deleted. In one embodiment, the genetically engineered bacterial strain is SYN-PKU708.

In one embodiment, the genetically engineered bacterial strain comprises a bacterial chromosome with three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of the mutated FNR transcription factor FNRS24Y knocked into the arabinose operon with expression driven by the native Para promoter and one copy of LAAD inserted at the same insertion site (Para::FNRS24Y-LAAD, e.g., SEQ ID NO: 73 of WO2017087580, the contents of which are herein incorporated by reference in their entirety), which is transcribed as a bicistronic message from the endogenous arabinose promoter. In one embodiment, the genetically engineered bacterial strain is SYN-PKU711.

In one embodiment, the genetically engineered bacterial strain comprises a bacterial chromosome comprising three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of LAAD knocked into the arabinose operon with expression driven by the native Para promoter (Para::LAAD, e.g., SEQ ID NO: 40 of WO2017087580, the contents of which are herein incorporated by reference in their entirety). The genome is further engineered to include a dapA auxotrophy, in which the dapA gene is deleted. In one embodiment, the genetically engineered bacterial strain is SYN-PKU709.

In one embodiment, the genetically engineered bacterial strain comprises a bacterial chromosome comprising three chromosomal insertions of FNR driven PAL3 (3×fnrS-PAL (malP/T, yicS/nepI, malE/K), e.g., SEQ ID NO: 38 of WO2017087580, the contents of which are herein incorporated by reference in their entirety) and two copies of FNR driven pheP (2×fnrS-pheP (lacZ, agaI/rsmI), e.g., SEQ ID NO: 62 of WO2017087580, the contents of which are herein incorporated by reference in their entirety). The strain further comprises one copy of the LAAD knocked into the arabinose operon with expression driven by the native Para promoter (Para::LAAD, e.g., SEQ ID NO: 40 of WO2017087580, the contents of which are herein incorporated by reference in their entirety). SYN-PKU710 further comprises two copies of IPTG inducible PAL3 (2×LacIPAL, exo/cea and rhtC/rhtB, e.g., SEQ ID NO: 74 of WO2017087580, the contents of which are herein incorporated by reference in their entirety), a dapA auxotrophy and is cured of all antibiotic resistances. In one embodiment, the genetically engineered bacterial strain is SYN-PKU710.

In any of these embodiments, the any of the genetically engineered described herein and depicted in FIG. 47 of WO2017087580, the contents of which are herein incorporated by reference in their entirety further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted.

In one embodiment, the genetically engineered bacteria comprise two additional copies of PheP (in addition to the wild type gene). This provides redundancy, in case one of the PheP genes acquires a mutation. In one embodiment, the PheP genes are inserted at lacZ and agal/rsml. In one embodiment, the two copies of PheP are under the control of the P_{fnrS} promoter. In one embodiment, the genetically engineered bacteria comprise three copies of PAL3. In one embodiment, the genetically engineered bacteria comprise three copies of PAL3, inserted at malEK, malPT, yicS/nepl. In one embodiment, the expression of the three copies of PAL3 is under the control of the P_{fnrS} promoter. In one embodiment, the genetically engineered bacteria comprise one or more copies of LAAD. In one embodiment, the genetically engineered bacteria comprise one copy of LAAD, inserted in the arabinose operon. In one embodiment, LAAD is under the control of the endogenous ParaBAD promoter. In one embodiment, the genetically engineered bacteria comprise an auxotrophy, e.g., deltaThyA. In one embodiment, the genetically engineered bacteria comprise an antibiotic resistance. In one embodiment, the genetically engineered bacteria comprise an antibiotic resistance and an auxotrophy, e.g., deltaThyA. In one embodiment, the genetically engineered bacteria do not comprise an auxotrophy, e.g., deltaThyA. In one embodiment, the genetically engineered bacteria do not comprise an antibiotic resistance. In one embodiment, the genetically engineered bacteria comprise neither an antibiotic resistance nor an auxotrophy, e.g., deltaThyA.

In one embodiment, the genetically engineered bacteria comprise three copies of PAL, e.g., PAL3, 2 copies of PheP (in addition to the endogenous PheP), and one copy of LAAD. In one embodiment, the genetically engineered bacteria comprise three copies of PAL, e.g., PAL3, 2 copies of PheP (in addition to the endogenous PheP), and one copy of LAAD, and an auxotrophy, e.g., delta ThyA. In one embodiment, the genetically engineered bacteria comprise three copies of PAL, 2 copies of PheP (in addition to the endogenous PheP), and one copy of LAAD, and an antibiotic resistance gene. In one embodiment, the genetically engineered bacteria comprise three copies of PAL, 2 copies of PheP (in addition to the endogenous PheP), and one copy of LAAD, and an antibiotic resistance gene and an auxotrophy, e.g., delta ThyA.

In one embodiment, the genetically engineered bacteria comprise three copies of PAL (each under control of a P_{fnrS} promoter), 2 copies of PheP (each under control of a P_{fnrS} promoter), and one copy of LAAD (under the control of the endogenous ParaBAD promoter). In one embodiment, the genetically engineered bacteria comprise three copies of PAL (each under control of a P_{fnrS} promoter), 2 copies of PheP (each under control of a P_{fnrS} promoter), and one copy of LAAD (under the control of the endogenous ParaBAD promoter), and an antibiotic resistance. In one embodiment, the genetically engineered bacteria comprise three copies of PAL (each under control of a P_{fnrS} promoter), 2 copies of PheP (each under control of a P_{fnrS} promoter), and one copy of LAAD (under the control of the endogenous ParaBAD promoter), and an auxotrophy, e.g., delta dapA. In one embodiment, the genetically engineered bacteria comprise three copies of PAL (each under control of a P_{fnrS} promoter), 2 copies of PheP (each under control of a P_{fnrS} promoter), and one copy of LAAD (under the control of the endogenous ParaBAD promoter), and an antibiotic resistance and an auxotrophy, e.g., deltadapA.

In one embodiment, the genetically engineered bacteria comprise three copies of PAL (each under control of a P_{fnrS} promoter and inserted at the malEK, malPT, and yicS/nepl sites), 2 copies of PheP (each under control of a P_{fnrS} promoter and inserted at the LacZ and agal/rsml sites), and one copy of LAAD (under the control of the endogenous ParaBAD promoter, and inserted in the endogenous arabinose operon). In one embodiment, the genetically engineered bacteria comprise three copies of PAL (each under control of a P_{fnrS} promoter and inserted at the malEK, malPT, and yicS/nepl sites), 2 copies of PheP (each under control of a P_{fnrS} promoter and inserted at the LacZ and agal/rsml sites), and one copy of LAAD (under the control of the endogenous ParaBAD promoter, and inserted in the endogenous arabinose operon), and further comprise an antibiotic resistance. In one embodiment, the genetically engineered bacteria comprise three copies of PAL (each under control of a P_{fnrS} promoter and inserted at the malEK, malPT, and yicS/nepl sites), 2 copies of PheP (each under control of a P_{fnrS} promoter and inserted at the LacZ and agal/rsml sites), and one copy of LAAD (under the control of the endogenous ParaBAD promoter, and inserted in the endogenous arabinose operon) and further comprise an auxotrophy, e.g., deltadapA. In one embodiment, the genetically engineered bacteria comprise three copies of PAL (each under control of a P_{fnrS} promoter and inserted at the malEK, malPT, and yicS/nepl sites), 2 copies of PheP (each under control of a P_{fnrS} promoter and inserted at the LacZ and agal/rsml sites), and one copy of LAAD (under the control of the endogenous ParaBAD promoter, and inserted in the endogenous arabinose operon), and further comprise an antibiotic resistance and an auxotrophy, e.g., deltadapA.

In one embodiment, the genetically engineered bacteria are SYN-PKU705. In one embodiment, SYN-PKU705 further comprises an antibiotic resistance. In one embodiment, SYN-PKU705 further comprises an auxotrophy, e.g., deltaThyA. In one embodiment, SYN-PKU705 further comprises an antibiotic resistance and auxotrophy, e.g., deltaThyA. In any of these embodiments, the any of the genetically engineered described in the preceeding paragraphs further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1001.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the dapA locus on the bacterial chromosome (low copy RBS;dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO:98 of WO2017087580, the contents of which are herein incorporated by reference in their entirety). In one embodiment, the strain is SYN-PKU1002.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1003.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1004.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1005

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1006.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1007.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1008.

In one embodiment, the genetically engineered bacterium a construct shown in FIG. 61C of WO2017087580, the contents of which are herein incorporated by reference in their entirety knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid shown in FIG. 61A, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1009.

In one embodiment, the genetically engineered bacterium a construct shown in FIG. 61C of WO2017087580, the contents of which are herein incorporated by reference in their entirety knocked into the dapA locus on the bacterial chromosome (low copy RBS;dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1010.

In one embodiment, the genetically engineered bacterium comprises a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1011.

In one embodiment, the genetically engineered bacterium a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1012.

In one embodiment, the genetically engineered bacterium comprises a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1013.

In one embodiment, the genetically engineered bacterium comprises a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65D of WO2017087580, the contents of which are herein incorporated by reference in their entirety (lacI-Ptac-PAL-PAL, e.g., SEQ ID NO: 98 of WO2017087580, the contents of which are herein incorporated by reference in their entirety). In one embodiment, the strain is SYN-PKU1014.

In one embodiment, the genetically engineered bacterium comprises a construct shown in FIG. 61D of WO2017087580, the contents of which are herein incorporated by reference in their entirety knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65B of WO2017087580, the contents of which are herein incorporated by reference in their entirety (LacI Fnrs-Ptac-PAL-PAL, e.g., SEQ ID NO: 97 of WO2017087580, the contents of which are herein incorporated by reference in their entirety). In one embodiment, the strain is SYN-PKU1015.

In one embodiment, the genetically engineered bacterium comprises a construct shown in FIG. 61D of WO2017087580, the contents of which are herein incorporated by reference in their entirety knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65D of WO2017087580, the contents of which are herein incorporated by reference in their entirety (lac-Ptac-PAL-PAL, e.g., SEQ ID NO: 98 of WO2017087580, the contents of which are herein incorporated by reference in their entirety). In one embodiment, the strain is SYN-PKU1016.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65A of WO2017087580, the contents of which are herein incorporated by reference in their entirety (Lac Fnrs-Ptac-PAL-PAL-PheP, e.g., SEQ ID NO: 95 of WO2017087580, the contents of which are herein incorporated by reference in their entirety). In one embodiment, the strain is SYN-PKU1017.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the dapA locus on the bacterial chromosome (low copy RBS;dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65C of WO2017087580, the contents of which are herein incorporated by reference in their entirety (Lac Fnrs-Ptac-PAL-PAL-PheP, e.g., SEQ ID NO: 96 of WO2017087580, the contents of which are herein incorporated by reference in their entirety). In one embodiment, the strain is SYN-PKU1018.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1019.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1020.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1021.

In one embodiment, the genetically engineered bacterium comprises two chromosomal copies of pheP (lacZ::PfnrS-pheP, agaI/rsmI::PfnrS-pheP) and a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1022.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1023.

In one embodiment, the genetically engineered bacterium comprises two chromosomal copies of pheP (lacZ::PfnrS-pheP, agaI/rsmI::PfnrS-pheP) and a construct shown in FIG. 61D of WO2017087580, the contents of which are herein incorporated by reference in their entirety knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1024.

In one embodiment, the genetically engineered bacterium a construct shown in FIG. 61C of WO2017087580, the contents of which are herein incorporated by reference in their entirety knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid shown in FIG. 61A, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1025.

In one embodiment, the genetically engineered bacterium a construct shown in FIG. 61C of WO2017087580, the contents of which are herein incorporated by reference in their entirety knocked into the dapA locus on the bacterial chromosome (low copy RBS;dapA::constitutive prom1 (BBA_J26100)-Pi(R6K)-constitutive promoter 2(P1)-Kis antitoxin). The strain further comprises a plasmid shown in FIG. 61A of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1026.

In one embodiment, the genetically engineered bacterium comprises a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1027.

In one embodiment, the genetically engineered bacterium a construct shown in FIG. 61D of WO2017087580, the contents of which are herein incorporated by reference in their entirety knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid shown in FIG. 61A, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1028.

In one embodiment, the genetically engineered bacterium comprises a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1029.

In one embodiment, the genetically engineered bacterium comprises a construct shown in FIG. 61C of WO2017087580, the contents of which are herein incorporated by reference in their entirety knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1030.

In one embodiment, the genetically engineered bacterium comprises a construct shown in FIG. 61D of WO2017087580, the contents of which are herein incorporated by reference in their entirety knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65A of WO2017087580, the contents of which are herein incorporated by reference in their entirety (LacI Fnrs-Ptac-PAL-PAL-PheP, e.g., SEQ ID NO: 95 of WO2017087580, the contents of which are herein incorporated by reference in their entirety). In one embodiment, the strain is SYN-PKU1032.

In one embodiment, the genetically engineered bacterium comprises a construct shown in FIG. 61D of WO2017087580, the contents of which are herein incorporated by reference in their entirety knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid shown in FIG. 61B of WO2017087580, the contents of which are herein incorporated by reference in their entirety, except that the bla gene is replaced with the construct of FIG. 65C of WO2017087580, the contents of which are herein incorporated by reference in their entirety (LacI Fnrs-Ptac-PAL-PAL-PheP, e.g., SEQ ID NO: 96 of WO2017087580, the contents of which are herein incorporated by reference in their entirety). In one embodiment, the strain is SYN-PKU1032. In any of these embodiments, the any of the genetically engineered described herein comprising an AIPS system further comprise a bacteriophage genome described herein, which further comprises one or more mutations described herein.

In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted.

In any of the preceeding embodiments, the bacteria described herein comprise one or more modifications or mutations, e.g., deletion, insertion, substitution or inversion, within the E. coli Nissle Phage 3 genome. In some embodiments, the mutation is an insertion. In some embodiments, the insertion comprises an antibiotic cassette as described herein. In some embodiments, the mutation is a deletion. In any of the embodiments described herein, the deletions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In one embodiment, the genetically engineered bacteria comprise one or more copies of PAL3, (e.g., under the control of a Pfnr promoter) and one or more copies of PAL1 (e.g. under the control of a Pfnr promoter). In one embodiment, the genetically engineered bacteria comprise one or more copies of PAL3, (e.g., under the control of a Pfnr promoter) and one or more copies of PAL1 (e.g. under the control of a Pfnr promoter); and further comprises one or more copies of a phenylalanine transporter (e.g., PheP and/or AroP, e.g., under the control of a Pfnr promoter). In one embodiment, the genetically engineered bacteria comprise one or more copies of PAL3, (e.g., under the control of a Pfnr promoter) and one or more copies of LAAD (e.g., under the control of the ParaBAD promoter). In one embodiment, the genetically engineered bacteria comprise one or more copies of PAL3, (e.g., under the control of a Pfnr promoter) and one or more copies of LAAD (e.g., under the control of the ParaBAD promoter); and are derived from E. coli Nissle and further comprises one or more copies of a phenylalanine transporter (e.g., PheP and/or AroP, e.g., under the control of a Pfnr promoter). In one embodiment, the genetically engineered bacteria comprise one or more copies of PAL3, (e.g., under the control of a Pfnr promoter) and one or more copies of PAH. In one embodiment, the genetically engineered bacteria comprise one or more copies of PAL3, (e.g., under the control of a Pfnr promoter) and one or more copies of PAH; and further comprises one or more copies of a phenylalanine transporter (e.g., PheP and/or AroP, e.g., under the control of a Pfnr promoter). In one embodiment, the genetically engineered bacteria comprise one or more copies of PAL1, (e.g., under the control of a Pfnr promoter) and one or more copies of LAAD (e.g., under the control of the ParaBAD promoter). In one embodiment, the genetically engineered bacteria comprise one or more copies of PAL1, (e.g., under the control of a Pfnr promoter) and one or more copies of LAAD (e.g., under the control of the ParaBAD promoter); and further comprises one or more copies of a phenylalanine transporter (e.g., PheP and/or AroP, e.g., under the control of a Pfnr promoter). In one embodiment, the genetically engineered bacteria comprise one or more copies of PAL1 (e.g., under the control of a Pfnr promoter) and one or more copies of PAH. In one embodiment, the genetically engineered bacteria comprise one or more copies of PAL1 (e.g., under the control of a Pfnr promoter) and one or more copies of PAH; and further comprises one or more copies of a phenylalanine transporter (e.g., PheP and/or AroP, e.g., under the control of a Pfnr promoter). In one embodiment, the genetically engineered bacteria comprise one or more copies of PAH and one or more copies of LAAD (e.g., under the control of the ParaBAD promoter). In one embodiment, the genetically engineered bacteria comprise one or more copies of PAH and one or more copies of LAAD (e.g., under the control of the ParaBAD promoter); and further comprises one or more copies of a phenylalanine transporter (e.g., PheP and/or AroP, e.g., under the control of a Pfnr promoter). PMEs and transporters may be integrated into any of the insertion sites described herein. In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise one or more copies of PAL3, e.g. (e.g., under the control of a Pfnr promoter), one or more copies of LAAD (e.g., under the control of the ParaBAD promoter), and one or more copies of PAH. In one embodiment, the genetically engineered bacteria comprise one or more copies of PAL3, e.g. (e.g., under the control of a Pfnr promoter), one or more copies of LAAD (e.g., under the control of the ParaBAD promoter), and one or more copies of PAH; and further comprise one or more copies of a phenylalanine transporter (e.g., PheP and/or AroP, e.g., under the control of a Pfnr promoter). In one embodiment, the genetically engineered bacteria comprise one or more copies of PAL3, e.g. (e.g., under the control of a Pfnr promoter), one or more copies of LAAD (e.g., under the control of the ParaBAD promoter), and one or more copies of PAL1 (e.g., under the control of a Pfnr promoter). In one embodiment, the genetically engineered bacteria comprise one or more copies of PAL3, e.g. (e.g., under the control of a Pfnr promoter), one or more copies of LAAD (e.g., under the control of the ParaBAD promoter), and one or more copies of PAL1 (e.g., under the control of a Pfnr promoter); and further comprise one or more copies of a phenylalanine transporter (e.g., PheP and/or AroP, e.g., under the control of a Pfnr promoter). In one embodiment, the genetically engineered bacteria comprise one or more copies of PAL3, e.g. (e.g., under the control of a Pfnr promoter), one or more copies of PAL1 (e.g., under the control of a Pfnr promoter), and one or more copies of PAH. In one embodiment, the genetically engineered bacteria comprise one or more copies of PAL3, e.g. (e.g., under the control of a Pfnr promoter), one or more copies of PAL1 (e.g., under the control of a Pfnr promoter), and one or more copies of PAH; and further comprise one or more copies of a phenylalanine transporter (e.g., PheP and/or AroP, e.g., under the control of a Pfnr promoter). In one embodiment, the genetically engineered bacteria comprise one or more copies of LAAD (e.g., under the control of the ParaBAD promoter), one or more copies of PAH, and one or more copies of PAL1 (e.g., under the control of an Pfnr promoter). In one embodiment, the genetically engineered bacteria comprise one or more copies of LAAD (e.g., under the control of the ParaBAD promoter), one or more copies of PAH, and one or more copies of PAL1 (e.g., under the control of an Pfnr promoter); and further comprise one or more copies of a phenylalanine transporter (e.g., PheP and/or AroP, e.g., under the control of a Pfnr promoter). PMEs and/or transporters may be integrated into any of the insertion sites described herein. Alternatively, PMEs and/or transporters may be comprised on low or high copy plasmids. PMEs and/or transporters may be integrated into any of the insertion sites described herein in combination with PMEs and/or transporters that are comprised on low or high copy plasmids. In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise one or more copies of PAL3, e.g. (e.g., under the control of a Pfnr promoter), one or more copies of PAL1, e.g. (e.g., under the control of a Pfnr promoter), one or more copies of LAAD (e.g., under the control of the ParaBAD promoter), and one or more copies of PAH. In one embodiment, the genetically engineered bacteria comprise one or more copies of PAL3, e.g. (e.g., under the control of a Pfnr promoter), one or more copies of PAL1, e.g. (e.g., under the control of a Pfnr promoter), one or more copies of LAAD (e.g., under the control of the ParaBAD promoter), and one or more copies of PAH; and further comprise one or more copies of a phenylalanine transporter (e.g., PheP and/or AroP, e.g., under the control of a Pfnr promoter). PMEs and transporters may be integrated into any of the insertion sites described herein. Alternatively, PMEs and/or transporters may be comprised on low or high copy plasmids. In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise one copy of PAL, (e.g., PAL1 or PAL3, e.g., under the control of a Pfnr promoter) one copy of PheP (e.g., under the control of a Pfnr promoter), and one copy of LAAD (e.g., under the control of the ParaBAD promoter). In one embodiment, the genetically engineered bacteria comprise one copy of PAL, (e.g., PAL1 or PAL3, e.g., under the control of a Pfnr promoter) two copies of PheP (e.g., under the control of a Pfnr promoter), and one copy of LAAD (e.g., under the control of the ParaBAD promoter). In one embodiment, the genetically engineered bacteria comprise one copy of PAL, (e.g., PAL1 or PAL3, e.g., under the control of a Pfnr promoter) one copy of PheP (e.g., under the control of a Pfnr promoter), and two copies of LAAD (e.g., under the control of the ParaBAD promoter). In one embodiment, the genetically engineered bacteria comprise one copy of PAL, (e.g., PAL1 or PAL3, e.g., under the control of a Pfnr promoter) two copies of PheP (e.g., under the control of a Pfnr promoter), and two copies of LAAD (e.g., under the control of the ParaBAD promoter). PMEs and transporters may be integrated into any of the insertion sites described herein. Alternatively, located PMEs and/or transporters may be comprised on low or high copy plasmids. In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise two copies of PAL (e.g., PAL1 or PAL3, e.g., under the control of a Pfnr promoter) one copy of PheP (e.g., under the control of a Pfnr promoter), and one copy of LAAD (e.g., under the control of the ParaBAD promoter). In one embodiment, the genetically engineered bacteria comprise two copies of PAL, (e.g., PAL1 or PAL3, e.g., under the control of a Pfnr promoter) two copies of PheP (e.g., under the control of a Pfnr promoter), and one copy of LAAD (e.g., under the control of the ParaBAD promoter). In one embodiment, the genetically engineered bacteria comprise two copies of PAL, (e.g., PAL1 or PAL3, e.g., under the control of a Pfnr promoter) one copy of PheP (e.g., under the control of a Pfnr promoter), and two copies of LAAD (e.g., under the control of the ParaBAD promoter). In one embodiment, the genetically engineered bacteria comprise two copies of PAL, (e.g., PAL1 or PAL3, e.g., under the control of a Pfnr promoter) two copies of PheP (e.g., under the control of a Pfnr promoter), and two copies of LAAD (e.g., under the control of the ParaBAD promoter). In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise three copies of PAL, (e.g., PAL1 or PAL3, e.g., under the control of a Pfnr promoter) one copy of PheP (e.g., under the control of a Pfnr promoter), and one copy of LAAD (e.g., under the control of the ParaBAD promoter). In one embodiment, the genetically engineered bacteria comprise three copies of PAL, (e.g., PAL1 or PAL3, e.g., under the control of a Pfnr promoter) two copies of PheP (e.g., under the control of a Pfnr promoter), and one copy of LAAD (e.g., under the control of the ParaBAD promoter). In one embodiment, the genetically engineered bacteria comprise three copies of PAL, (e.g., PAL1 or PAL3, e.g., under the control of a Pfnr promoter) one copy of PheP (e.g., under the control of a Pfnr promoter), and two copies of LAAD (e.g., under the control of the ParaBAD promoter). In one embodiment, the genetically engineered bacteria comprise three copies of PAL, (e.g., PAL1 or PAL3, e.g., under the control of a Pfnr promoter) two copies of PheP (e.g., under the control of a Pfnr promoter), and two copies of LAAD (e.g., under the control of the ParaBAD promoter). In one embodiment, the genetically engineered bacteria comprise three copies of PAL, (e.g., PAL1 or PAL3, e.g., under the control of a Pfnr promoter), three copies of PheP (e.g., under the control of a Pfnr promoter), and two copies of LAAD (e.g., under the control of the ParaBAD promoter). In one embodiment, the genetically engineered bacteria comprise three copies of PAL, (e.g., PAL1 or PAL3, e.g., under the control of a Pfnr promoter), three copies of PheP (e.g., under the control of a Pfnr promoter), and one copy of LAAD (e.g., under the control of the ParaBAD promoter). In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise four copies of PAL, (e.g., PAL1 or PAL3, e.g., under the control of a Pfnr promoter) one copy of PheP (e.g., under the control of a Pfnr promoter), and one copy of LAAD (e.g., under the control of the ParaBAD promoter). In one embodiment, the genetically engineered bacteria comprise four copies of PAL, (e.g., PAL1 or PAL3, e.g., under the control of a Pfnr promoter) two copies of PheP (e.g., under the control of a Pfnr promoter), and one copy of LAAD (e.g., under the control of the ParaBAD promoter). In one embodiment, the genetically engineered bacteria comprise four copies of PAL, (e.g., PAL1 or PAL3, e.g., under the control of a Pfnr promoter) one copy of PheP (e.g., under the control of a Pfnr promoter), and two copies of LAAD (e.g., under the control of the ParaBAD promoter). In one embodiment, the genetically engineered bacteria comprise four copies of PAL, (e.g., PAL1 or PAL3, e.g., under the control of a Pfnr promoter) two copies of PheP (e.g., under the control of a Pfnr promoter), and two copies of LAAD (e.g., under the control of the ParaBAD promoter). In any of these embodiments, the any of the genetically engineered described in the preceding paragraph further comprise a bacteriophage genome described herein, which further comprises one or more mutations described herein. In any of these embodiments, the genetically engineered bacteria are derived from E. coli Nissle and further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted. In one embodiment, the genetically engineered bacteria comprise five copies of PAL, (e.g., PAL1 or PAL3, e.g., under the control of a Pfnr promoter) one copy of PheP (e.g., under the control of a Pfnr promoter), and one copy of LAAD (e.g., under the control of the ParaBAD promoter). In one embodiment, the genetically engineered bacteria comprise five copies of PAL, (e.g., PAL1 or PAL3, e.g., under the control of a Pfnr promoter) two copies of PheP (e.g., under the control of a Pfnr promoter), and one copy of LAAD (e.g., under the control of the ParaBAD promoter). In one embodiment, the genetically engineered bacteria comprise five copies of PAL, (e.g., PAL1 or PAL3, e.g., under the control of a Pfnr promoter) one copy of PheP (e.g., under the control of a Pfnr promoter), and two copies of LAAD (e.g., under the control of the ParaBAD promoter). In one embodiment, the genetically engineered bacteria comprise five copies of PAL, (e.g., PAL1 or PAL3, e.g., under the control of a Pfnr promoter) two copies of PheP (e.g., under the control of a Pfnr promoter), and two copies of LAAD (e.g., under the control of the ParaBAD promoter). In any of these embodiments, the genetically engineered bacteria further comprise a bacteriophage genome described herein, which further comprises one or more mutations described herein. In a non-limiting example, the phage genome is Phage 3 and one or more genes are partially deleted. In a non-limiting example, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170 are partially or completely deleted.

In any of these embodiments, the bacteria described herein comprise one or more modifications or mutations, e.g., deletion, insertion, substitution or inversion, within the E. coli Nissle Phage 3 genome. In some embodiments, the mutation is an insertion. In some embodiments, the insertion comprises an antibiotic cassette as described herein. In some embodiments, the mutation is a deletion. In any of the embodiments described herein, the deletions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise one or more of the following elements:
a) One or more additional copies of an endogenous Nissle gene encoding a high affinity phenylalanine transporter (PheP);
b) One more copies of a gene encoding phenylalanine ammonia lyase (PAL) derived from Photorhabdus;
c) One or more gene encoding L-amino acid deaminase (LAAD) derived from Proteus mirabilis;
d) One or more deletion of endogenous genes to generate an auxotrophy, e.g., dapA or ThyA;
e) Antibiotic resistance; and
f) One or more modifications or mutations, e.g., deletion, insertion, substitution or inversion, within the E. coli Nissle Phage 3 genome

In any of the preceding embodiments, the mutation in Phage 3 is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise one or more of the following elements:
a) One or more additional copies of an endogenous Nissle gene encoding a high affinity phenylalanine transporter (PheP);
b) One more copies of a gene encoding phenylalanine ammonia lyase (PAL) derived from Photorhabdus;
c) One or more gene encoding L-amino acid deaminase (LAAD) derived from Proteus mirabilis;
d) One or more deletion of endogenous genes to generate an auxotrophy, e.g., dapA or ThyA; and
e) One or more modifications or mutations, e.g., deletion, insertion, substitution or inversion, within the E. coli Nissle Phage 3 genome.

In any of the preceding embodiments, the mutation in Phage 3 is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise the following elements:
a) One or more additional copies of an endogenous Nissle gene encoding a high affinity phenylalanine transporter (PheP);
b) One more copies of a gene encoding phenylalanine ammonia lyase (PAL) derived from Photorhabdus;
c) One or more gene encoding L-amino acid deaminase (LAAD) derived from Proteus mirabilis;
d) One or more deletion of endogenous genes to generate an auxotrophy, e.g., dapA or ThyA; and
e) One or more modifications or mutations, e.g., deletion, insertion, substitution or inversion, within the E. coli Nissle Phage 3 genome.

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise one or more of the following elements:
a) One or more additional copies of an endogenous Nissle gene encoding a high affinity phenylalanine transporter (PheP) under the regulatory control of an promoter inducible under exogenous environmental conditions;
b) One more copies of a gene encoding phenylalanine ammonia lyase (PAL) derived from Photorhabdus luminescens inducible under exogenous environmental conditions;
c) One or more copies of the gene encoding PAL under the regulatory control of a promoter inducible by a chemical or nutritional inducer;
d) One or more gene encoding L-amino acid deaminase (LAAD) derived from Proteus mirabilis under the regulatory control of a promoter inducible by a chemical inducer, which is the same or different from the chemical inducer that induces expression of PAL;
e) One or more deletion of endogenous genes to generate an auxotrophy, e.g., dapA or ThyA; and
f) One or more modifications or mutations, e.g., deletion, insertion, substitution or inversion, within the E. coli Nissle Phage 3 genome

In any of the preceding embodiments, the mutation in Phage 3 is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise the following elements:
a) One or more additional copies of an endogenous Nissle gene encoding a high affinity phenylalanine transporter (PheP) under the regulatory control of an promoter inducible under exogenous environmental conditions;
b) One more copies of a gene encoding phenylalanine ammonia lyase (PAL) derived from Photorhabdus luminescens inducible under exogenous environmental conditions;
c) One or more copies of the gene encoding PAL under the regulatory control of a promoter inducible by a chemical or nutritional inducer;
d) One or more gene encoding L-amino acid deaminase (LAAD) derived from Proteus mirabilis under the regulatory control of a promoter inducible by a chemical inducer, which is the same or different from the chemical inducer that induces expression of PAL;
e) One or more deletion of endogenous genes to generate an auxotrophy, e.g., dapA or ThyA; and
f) One or more modifications or mutations, e.g., deletion, insertion, substitution or inversion, within the E. coli Nissle Phage 3 genome

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise the following elements:
a) Two additional copies of an endogenous Nissle gene encoding a high affinity phenylalanine transporter (PheP) under the regulatory control of an promoter inducible under exogenous environmental conditions;
b) Three copies of a gene encoding phenylalanine ammonia lyase (PAL) derived from Photorhabdus luminescens inducible under exogenous environmental conditions;
c) Two copies of the gene encoding PAL under the regulatory control of a promoter inducible by a chemical or nutritional inducer;
d) One copy of the gene encoding L-amino acid deaminase (LAAD) derived from Proteus mirabilis under the regulatory control of a promoter inducible by a chemical inducer, which is the same or different from the chemical inducer that induces expression of PAL;
e) One or more deletion of endogenous dapA to generate an auxotrophy; and
f) one or more modifications or mutations, e.g., deletion, insertion, substitution or inversion, within the E. coli Nissle Phage 3 genome

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacteria comprise one or more of the following elements:
a) One or more additional copies of an endogenous Nissle gene encoding a high affinity phenylalanine transporter (PheP) under the regulatory control of an anaerobic-inducible promoter (PfnrS) and the anaerobic-responsive transcriptional activator FNR;
b) One more copies of a gene encoding phenylalanine ammonia lyase (PAL) derived from Photorhabdus luminescens under the regulatory control of PfnrS and FNR;
c) One or more copies of the gene encoding PAL under the regulatory control of a synthetic promoter (Ptac) and the lactose-responsive transcriptional repressor LacI;
d) One or more gene encoding L-amino acid deaminase (LAAD) derived from Proteus mirabilis under the regulatory control of the arabinose-inducible promoter (PBAD) and the arabinose-responsive transcriptional activator AraC;
c) One or more deletion of endogenous genes to generate an auxotrophy, e.g., dapA or ThyA; and
f) one or more modifications or mutations, e.g., deletion, insertion, substitution or inversion, within the E. coli Nissle Phage 3 genome.

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In some embodiments, the genetically engineered bacterium comprises the following elements:
a) One or more additional copies of an endogenous Nissle gene encoding a high affinity phenylalanine transporter (PheP) under the regulatory control of an anaerobic-inducible promoter (PfnrS) and the anaerobic-responsive transcriptional activator FNR;
b) One more copies of a gene encoding phenylalanine ammonia lyase (PAL) derived from Photorhabdus luminescens under the regulatory control of PfnrS and FNR;
c) One or more copies of the gene encoding PAL under the regulatory control of a synthetic promoter (Ptac) and the lactose-responsive transcriptional repressor LacI;
d) One or more gene encoding L-amino acid deaminase (LAAD) derived from Proteus mirabilis under the regulatory control of the arabinose-inducible promoter (PBAD) and the arabinose-responsive transcriptional activator AraC;
e) One or more deletion of endogenous genes to generate an auxotrophy, e.g., dapA or ThyA; and
f) one or more modifications or mutations, e.g., deletion, insertion, substitution or inversion, within the E. coli Nissle Phage 3 genome

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In one specific embodiment, the genetically engineered bacteria comprise each of the following elements:
a) two additional copies of the endogenous Nissle gene encoding the high affinity phenylalanine transporter (PheP) under the regulatory control of an anaerobic-inducible promoter (PfnrS) and the anaerobic-responsive transcriptional activator FNR, inserted into the chromosome at the lacZ and the agal/rsml loci;
b) three copies of a gene encoding phenylalanine ammonia lyase (PAL) derived from Photorhabdus luminescens under the regulatory control of PfnrS and FNR, inserted into the chromosome at the malEK, malPT, and yicS/nepI loci;
c) two additional copies of the gene encoding PAL under the regulatory control of a synthetic promoter (Plac) and the lactose-responsive transcriptional repressor LacI, inserted at the exo/cea and the rhtC/rhtB loci;
d)
e) one copy of a gene encoding L-amino acid deaminase (LAAD) derived from Proteus mirabilis under the regulatory control of the arabinose-inducible promoter (PBAD) and the arabinose-responsive transcriptional activator AraC, with LAAD under the native arabinose promoter;
f) a deletion of the dapA gene that encodes 4-hydroxy-tetrahydropicolinate synthase to create a diaminopimelate auxotroph; and One or more modifications or mutations, e.g., deletion, insertion, substitution or inversion, within the E. coli Nissle Phage 3 genome

In some of the preceding embodiments, the mutation is an insertion. In some embodiments, the insertion comprises an antibiotic cassette. In some of the preceding embodiments, the mutation is a deletion. In any one the of the preceding embodiments, the deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

In one specific embodiment, the genetically engineered bacteria comprise each of the following elements:
a) Two additional copies of the endogenous Nissle gene encoding the high affinity phenylalanine transporter (PheP) under the regulatory control of an anaerobic-inducible promoter (PfnrS) and the anaerobic-responsive transcriptional activator FNR, inserted into the chromosome at the lacZ and the agal/rsml loci;
b) Three copies of a gene encoding phenylalanine ammonia lyase (PAL) derived from Photorhabdus luminescens under the regulatory control of PfnrS and FNR, inserted into the chromosome at the malEK, malPT, and yicS/nepI loci;
c) Two additional copies of the gene encoding PAL under the regulatory control of a synthetic promoter (Plac) and the lactose-responsive transcriptional repressor LacI, inserted at the exo/cea and the rhtC/rhtB loci;
d) One copy of a gene encoding L-amino acid deaminase (LAAD) derived from Proteus mirabilis under the regulatory control of the arabinose-inducible promoter (PBAD) and the arabinose-responsive transcriptional activator AraC, with LAAD under the native arabinose promoter;
e) A deletion of the dapA gene that encodes 4-hydroxy-tetrahydropicolinate synthase to create a diaminopimelate auxotroph; and
f) A deletion of a region within the E. coli Nissle Phage 3 genome, wherein deleted region comprises ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, and partially ECOLIN_10165.

In one specific embodiment, the genetically engineered bacteria comprise each of the following elements:
a) two additional copies of the endogenous Nissle gene encoding the high affinity phenylalanine transporter (PheP) under the regulatory control of an anaerobic-inducible promoter (PfnrS) and the anaerobic-responsive transcriptional activator FNR, inserted into the chromosome at the lacZ and the agal/rsml loci;
b) three copies of a gene encoding phenylalanine ammonia lyase (PAL) derived from Photorhabdus luminescens under the regulatory control of PfnrS and FNR, inserted into the chromosome at the malEK, malPT, and yicS/nepI loci;
c) two additional copies of the gene encoding PAL under the regulatory control of a synthetic promoter (Plac) and the lactose-responsive transcriptional repressor LacI, inserted at the exo/cea and the rhtC/rhtB loci;
d) one copy of a gene encoding L-amino acid deaminase (LAAD) derived from Proteus mirabilis under the regulatory control of the arabinose-inducible promoter (PBAD) and the arabinose-responsive transcriptional activator AraC, with LAAD under the native arabinose promoter;
e) a deletion of the dapA gene that encodes 4-hydroxy-tetrahydropicolinate synthase to create a diaminopimelate auxotroph; and
g) a deletion of a region within the E. coli Nissle Phage 3 genome, wherein deleted region consists of SEQ ID NO: 130.
   In one embodiment, the genetically engineered bacterium is E. coli Nissle. In some embodiments, the strain comprises the same modifications as SYN-PKU-2002. In some embodiments, the strain is SYN-PKU-2002.

Table 14 contains non-limiting examples of the genetically engineered bacteria of the disclosure. In certain embodiments, the genetically engineered bacteria of Table 14 further contain a PME for secretion.

TABLE 14
Non-limiting Examples of Embodiments of the Disclosure

| Strain Name | Genotype |
|---|---|
| SYN-PKU101 | Low copy pSC101-Ptet::PAL1, ampicillin resistant |
| SYN-PKU102 | High copy pColE1-Ptet::PAL1, ampicillin resistant, |
| SYN-PKU201 | Low copy pSC101-Ptet::PAL3, ampicillin resistant |
| SYN-PKU202 | High copy pColE1-Ptet::PAL3, ampicillin resistant, |
| SYN-PKU203 | lacZ::Ptet-pheP::cam |
| SYN-PKU401 | Low copy pSC101-Ptet::PAL1, ampicillin resistant, |
| | chromosomal lacZ::Ptet-pheP::cam |
| SYN-PKU402 | High copy pColE1-Ptet::PAL1, ampicillin resistant, |
| | chromosomal lacZ::Ptet-pheP::cam |
| SYN-PKU302 | Low Copy pSC101-Ptet::PAL3, ampicillin resistant; |
| | chromosomal lacZ::Ptet-pheP::cam |
| SYN-PKU303 | High copy pColE1-Ptet::PAL3, ampicillin resistant, |
| | chromosomal lacZ::Ptet-pheP::cam |
| SYN-PKU304 | Low Copy pSC101-PfnrS-PAL3, ampicillin resistant; |
| | chromosomal lacZ::PfnrS-pheP::cam |
| SYN-PKU305 | Low Copy pSC101-PfnrS-PAL3, kanamycin resistant; |
| | chromosomal lacZ::PfnrS-pheP::cam |
| SYN-PKU306 | Low Copy pSC101-PfnrS-PAL3, kanamycin resistant; |
| | thyA |
| SYN-PKU307 | Low Copy pSC101-PfnrS-PAL3, ampicillin resistant; |
| SYN-PKU308 | Low Copy pSC101-PfnrS-PAL3, kanamycin resistant; |
| SYN-PKU401 | High Copy pUC57-Ptet-LAAD; kanamycin resistant |
| SYN-PKU501 | malPT:: PfnrS-PAL3::kan |
| SYN-PKU502 | malPT:: PfnrS-PAL3::kan; bicistronic lacZ:: PfnrS- |
| | PAL3-pheP::cam |
| SYN-PKU503 | malEK::PfnrS-PAL3::cam |
| SYN-PKU504 | agaI/rsmI::PfnrS-PAL3 |
| SYN-PKU505 | cea::PfnrS-PAL3 |
| SYN-PKU506 | malEK::PfnrS-PAL3; agaI/rsmI::PfnrS-PAL3; |
| | cea::PfnrS-PAL3 |
| SYN-PKU507 | malEK:: PfnrS-PAL3; agaI/rsmI::PfnrS-PAL3; |
| | cea::PfnrS-PAL3; lacZ::PfnrS-pheP::cam |
| SYN-PKU508 | malEK::PfnrS-PAL3; pheA auxotroph |
| SYN-PKU509 | malEK::PfnrS-PAL3; agaI/rsmI::PfnrS-PAL3; |
| | cea::PfnrS-PAL3; lacZ::PfnrS-pheP::cam |
| SYN-PKU601 | malPT::PfnrS-INT5::kan, rrnBUP -[PAL3]; |
| | lacZ::PfnrS-pheP::cam (recombinase based strain) |
| SYN-PKU510 | malEK::PfnrS-PAL3; agaI/rsmI::PfnrS-PAL3; |
| | cea::PfnrS-PAL3; |
| SYN-PKU511 | malEK:: PfnrS-PAL3; agaI/rsmI::PfnrS-PAL3; |
| | cea::PfnrS-PAL3; yicS/nepI::PfnrS-PAL3::kan; |
| | malPT::PfnrS-PAL3; lacZ::PfnrS-pheP; ΔthyA |
| SYN-PKU204 | lacZ::PfnrS-pheP::cam |
| SYN-PKU512 | malEK::PfnrS-PAL3; agaI/rsmI::PfnrS-PAL3; |
| | cea::PfnrS-PAL3; malPT::PfnrS-PAL3; lacZ::PfnrS- |
| | pheP::cam; ΔthyA |
| SYN-PKU513 | malEK:: PfnrS-PAL3; agaI/rsmI::PfnrS-PAL3; |
| | cea::PfnrS-PAL3; lacZ::PfnrS-pheP; ΔthyA |
| SYN-PKU514 | malEK:: PfnrS-PAL3; agaI/rsmI:PfnrS-PAL3; |
| | cea::PfnrS-PAL3; malPT::PfnrS-PAL3; ΔthyA |
| SYN-PKU515 | malEK:: PfnrS-PAL3; agaI/rsmI::PfnrS-PAL3; |
| | cea::PfnrS-PAL3; ΔthyA |
| SYN-PKU516 | agaI/rsmI::PfnrS-PAL3::kan |
| SYN-PKU517 | malEK:: PfnrS-PAL3::cam; malPT::PfnrS- |
| | PAL3::kan; lacZ::PfnrS-pheP; ΔthyA |
| SYN-PKU518 | malEK-PfnrS-PAL3::cam; PfnrS::pheP::kan |
| SYN-PKU519 | ParaBC-PAL3::cam; PfnrS-pheP::kan |
| SYN-PKU520 | agaI/rsmI:PfnrS-PAL3::kan; PfnrS-PheP::cam |
| SYN-PKU801 | ΔargR; thyA::cam |
| SYN-PKU701 | ParaBC-LAAD::cam; malEK-PfnrS-PAL3; |
| | malPT::PfnrS-PAL3::kan; PfnrS-pheP |
| SYN-PKU521 | yicS/nepI::PfnrS-PAL3::kan; lacZ::PfnrS-pheP::cam |
| SYN-PKU522 | cea::PfnrS-PAL3::kan; lacZ::PfnrS-pheP::cam |
| SYN-PKU523 | malPT::PfnrS-PAL3::kan; lacZ::PfnrS-pheP::cam |
| SYN-PKU524 | malEK:: PfnrS-PAL3; malPT::PfnrS-PAL3; |
| | lacZ::PfnrS-pheP |
| SYN-PKU702 | malEK:: PfnrS-PAL3; lacZ::PfnrS-pheP; Para::LAAD |
| SYN-PKU703 | malEK:: PfnrS-PAL3; malPT::PfnrS-PAL3; |
| | lacZ::PfnrS-pheP; agaI/rsmI:PfnrS::pheP; |
| | Para::LAAD |
| SYN-PKU704 | malEK:: PfnrS-PAL3; malPT::PfnrS-PAL3; |
| | yicS/nepI::PfnrS-PAL3; lacZ::PfnrS-pheP; |
| | Para::LAAD |
| SYN-PKU705 | malEK:: PfnrS-PAL3; malPT::PfnrS-PAL3; |
| | yicS/nepI::PfnrS-PAL3::kan; lacZ::PfnrS-pheP; |
| | agaI/rsmI::PfnrS::pheP Para::LAAD |
| SYN-PKU602 | malEK:: PT7::PAL3; Para::INT5::cam |
| | (recombinase); lacZ::PfnrS-pheP; |
| | malPT::Pconstitutive::T7 polymerase (unflipped); |
| SYN-PKU901 | Nissle with streptomycin resistance |
| SYN001 | WT Nissle |
| SYN-902 | WT nissle that has the pKD46 plasmid |
| SYN-903 | WT nissle that has the pKD46 plasmid and with phage |
| | knockout |
| SYN-PKU713 | LacZ::PfnrS-PAL3::pheP |
| SYN-PKU706 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::LAAD, ΔdapA::cm |
| SYN-PKU707 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::FNRS24Y::cm |
| SYN-PKU708 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::FNRS24Y-LAAD; |
| | ΔdapA |
| SYN-PKU-709 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para-LAAD; ΔdapA |
| SYN-PKU-710 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::LAAD; exo/cea:: |
| | LacIPAL3; rhtC/rhtB::LacIPAL3; ΔdapA |
| SYN-PKU711 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::FNRS24Y-LAAD; |
| SYN-PKU-712 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::FNRS24Y; ΔDapA |
| SYN-PKU-713 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::LAAD; exo/cea:: |
| | LacIPAL3:cm; rhtC/rhtB::LacIPAL3:kn; ΔdapA |
| | (chloramphenicol and kanamycin resistance) |
| SYN-PKU-2002 | malEK:: PfnrS-PAL3; malPT::PfnrS-PAL3; |
| | yicS/nepI:: PfnrS-PAL3; lacZ::PfnrS-pheP, |
| | agaI/rsmI::PfnrS-pheP; Para::LAAD; exo/cea:: |
| | LacIPAL3; rhtC/rhtB::LacIPAL3; ΔdapA ; phage free |
| | (deletion of SEQ ID NO: 130) |
| SYN-PKU-2001 | Parental strain of SYN2002 with insertion of phage3 |
| | KO frag::cm and cured of pKD46 (not cured of |
| | chloramphenicol resistance); malEK:: PfnrS-PAL3; |
| | malPT::PfnrS-PAL3; yicS/nepI:: PfnrS-PAL3; |
| | lacZ::PfnrS-pheP, agaI/rsmI::PfnrS-pheP; |
| | Para::LAAD; exo/cea:: LacIPAL3; |
| | rhtC/rhtB::LacIPAL3; ΔdapA) with insertion of |
| | phage3 KO frag::cm and cured of pKD46 (not cured |
| | of chloramphenicol resistance) |
| SYN3282 | E. coli Nissle 1917, lacZ::P_{fnrS}-PheP, ΔlacZ, |
| | malEK::P_{fnrs}-PAL, araBC::P_{BAD}-LAAD, malPT::P_{fnrS}- |
| | PAL, yicS/nepI::P_{fnrS}-PAL, agaI/rsmI::P_{fnrS}-PheP, |
| | exo/cea::P_{tac}-PAL, rhtBC::P_{tac}-PAL, ΔΦ, |
| | chloramphenicol resistant |
| | Note: DAP prototrophic strain genetically engineered |
| | to encode Phe-degrading activity |
| SYN766 | E. coli Nissle 1917, ΔdapA, chloramphenicol resistant. |

In any of the embodiments, described herein, in which the genetically engineered organism, e.g., engineered bacteria or engineered OV, produces a protein, polypeptide, peptide, or other anti-cancer, gut barrier enhancer, anti-inflammatory, neuromodulatory, satiety effector, DNA, RNA, small molecule or other molecule intended to be secreted from the microorganism, the engineered microorganism may comprise a secretion mechanism and corresponding gene sequence(s) encoding the secretion system.

| | |
|---|---|
| SYN-PKU-1035 | SYN-PKU-1033 cured entirely |
| SYN-PKU-1036 | SYN-PKU-1034 cured entirely |

In one embodiment, the genetically engineered bacteria comprise one or more PMEs for metabolizing phenylalanine in combination with one or more PMEs for secretion. In one embodiment, the genetically engineered bacteria comprise one or more PMEs for metabolizing phenylalanine and a phenylalanine transporter in combination with one or more PMEs for secretion. In one embodiment, the genetically engineered bacteria comprise one or more PMEs for metabolizing phenylalanine and a phenylalanine transporter in combination with one or more PMEs for secretion, and also include an auxotrophy and/or an antibiotic resistance. Secretion systems described herein are utilized to secrete the PMEs in the genetically engineered bacteria with multiple mechanisms of action.

In any of these embodiments, the bacteria described herein comprise one or more modifications or mutations, e.g., deletion, insertion, substitution or inversion, within the E. coli Nissle Phage 3 genome. In some embodiments, the mutation is an insertion. In some embodiments, the mutation is a deletion. In any of the embodiments described herein, the deletions encompass (completely or partially) or are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the deletion is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281.

### Secretion

In any of the embodiments described herein, in which the genetically engineered microorganism produces a protein, polypeptide, peptide, or other anti-cancer, immune modulatory, DNA, RNA, small molecule or other molecule intended to be secreted from the microorganism, the engineered microorganism may comprise a secretion mechanism and corresponding gene sequence(s) encoding the secretion system.

In some embodiments, the genetically engineered bacteria further comprise a native secretion mechanism or non-native secretion mechanism that is capable of secreting the anti-cancer molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

Suitable secretion systems for secretion of heterologous polypeptides, e.g., effector molecules, from gram negative and gram positive bacteria are described in pending, co-owned International Patent Applications PCT/US2016/34200, filed May 25, 2016, PCT/US2017/013072, filed Jan. 11, 2017, PCT/US2017/016603, filed Feb. 3, 2017, PCT/US2017/016609, filed Feb. 4, 2016, PCT/US2017/017563, filed Feb. 10, 2017, PCT/US2017/017552, filed Feb. 10, 2017, PCT/US2016/044922, filed Jul. 29, 2016, PCT/US2016/049781, filed Aug. 31, 2016, PCT/US2016/37098, filed Jun. 10, 2016, PCT/US2016/069052, filed Dec. 28, 2016, PCT/US2016/32562, filed May 13, 2016, PCT/US2016/062369, filed Nov. 16, 2016, and PCT/US2017/013072, the contents of which are herein incorporated by reference in their entireties.

### Surface Display

In some embodiments, the genetically engineered bacteria and/or microorganisms encode one or more gene(s) and/or gene cassette(s) encoding a polypeptide of interest described herein which is anchored or displayed on the surface of the bacteria and/or microorganisms. Examples of the payload molecules which are displayed or anchored to the bacteria and/or microorganism, are any of the payload molecules or other effectors described herein, and include but are not limited to enzymes (e.g., PME(s) or kynureninase), antibodies, e.g., scFv fragments, and tumor-specific antigens or neoantigens.

Suitable systems for surface display of heterologous polypeptides, e.g., effector molecules, on the surface of gram negative and gram positive bacteria are described in International Patent Application PCT/US2017/013072, filed Jan. 11, 2017, published as WO2017/123675, the contents of which is herein incorporated by reference in its entirety

### The Essential Genes and Auxotrophs

As used herein, the term “essential gene” refers to a gene that is necessary for cell growth and/or survival. Bacterial essential genes are well known to one of ordinary skill in the art, and can be identified by directed deletion of genes and/or random mutagenesis and screening (see, e.g., Zhang and Lin, “DEG 5.0, a database of essential genes in both prokaryotes and eukaryotes,” Nucl Acids Res, 2009; 37:D455-D458 and Gerdes et al., “Essential genes on metabolic maps,” Curr Opin Biotechnol, 2006; 17(5):448-456, the entire contents of each of which are expressly incorporated herein by reference).

An “essential gene” may be dependent on the circumstances and environment in which an organism lives. For example, a mutation of, modification of, or excision of an essential gene may result in the genetically engineered bacteria of the disclosure becoming an auxotroph. An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is a DNA synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, ginA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thiI, as long as the corresponding wild-type gene product is not produced in the bacteria. Table 18A lists exemplary bacterial genes which may be disrupted or deleted to produce an auxotrophic strain. These include, but are not limited to, genes required for oligonucleotide synthesis, amino acid synthesis, and cell wall synthesis.

TABLE 18A
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 19A shows the survival of various amino acid auxotrophs in the mouse gut, as detected 24 hrs and 48 hrs post-gavage. These auxotrophs were generated using BW25113, a non-Nissle strain of E. coli.

TABLE 19A
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/Leucine | Present | Present | Absent |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thymidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro, or in the presence of high DAP levels found naturally in the human gut in vivo. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro, or in the presence of high uracil levels found naturally in the human gut in vivo. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria of the invention comprise a deletion or mutation in two or more genes required for cell survival and/or growth.

Other examples of essential genes include, but are not limited to, yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, lgt, fbaA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, ispA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, injB, nusA, ftsH, obgE, rpmA, rplU, ispB, murA, yrbB, yrbK, yhbN, rpsl, rplm, degS, mreD, mreC, mreB, accB, accC, yrdC, def, fint, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fidA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, oE, purB, ymfK, minE, mind, pth, rsA, ispE, lolB, hemA, prfA, prmC, kdsA, topA, ribA, fabL, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson, “Synthetic Auxotrophs with Ligand-Dependent Essential Genes for a BL21 (DE3) Biosafety Strain,” ACS Synth Biol 2015; 4(12):1279-1286, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG, and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A, and F40G. In some embodiments, the essential gene is tyrS comprising the mutations L36V, C38A, and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I, and L6G. In some embodiments, the essential gene is adk comprising the mutations I4L, L51, and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid, or L-histidine methyl ester. Bacterial cells comprising mutations in dnaN (H191N, R240C, I317S, F319V, L340T, V347I, and S345C) are complemented by benzothiazole, indole, or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system shown in FIGS. 85-86 of WO2017087580.

In some embodiments, the genetically engineered bacterium of the disclosure is an auxotroph and also comprises kill switch circuitry, such as any of the kill switch components and systems described herein. For example, the genetically engineered bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synth Biol, 2015; 4(3):307-316, the entire contents of which are expressly incorporated herein by reference). In some embodiments, the genetically engineered bacterium of the disclosure is an auxotroph and also comprises kill switch circuitry, such as any of the kill switch components and systems described herein, as well as another biosecurity system, such a conditional origin of replication (Wright et al., 2015). In one embodiment, a genetically engineered bacterium, comprises one or more AIPS constructs integrated into the bacterial chromosome in combination with one or more biosafety plasmid(s). In some embodiments, the plasmid comprises a conditional origin of replication (COR), for which the plasmid replication initiator protein is provided in trans, i.e., is encoded by the chromosomally integrated biosafety construct. In some embodiments, the chromosomally integrated construct is further introduced into the host such that an auxotrophy results (e.g., dapA or thyA auxotrophy), which in turn is complemented by a gene product expressed from the biosafety plasmid construct. In some embodiments, the biosafety plasmid further encodes a broad-spectrum toxin (e.g., Kis), while the integrated biosafety construct encodes an anti-toxin (e.g., anti-Kis), permitting propagation of the plasmid in the bacterial cell containing both constructs. Without wishing to be bound by theory, this mechanism functions to select against plasmid spread by making the plasmid DNA itself disadvantageous to maintain by a wild-type bacterium. A non-limiting example of such a biosafety system is shown in FIG. 61A, FIG. 61B, FIG. 61C, and FIG. 61D of WO2017087580, the contents of which are herein incorporated by reference in their entirety.

In some embodiments, the genetically engineered bacteria comprise a chromosomally inserted biosafety construct nucleic acid sequence (to be combined with a plasmid based biosafety construct) that is at least about 80%, at least about 85%, at least about 90%, at least about 95%, or at least about 99% homologous to the DNA sequence of SEQ ID NO: 81, 82, 83, 84, 85 of WO2017087580, the contents of which are herein incorporated by reference in their entirety, or a functional fragment thereof. In some embodiments, the genetically engineered bacteria comprise a chromosomally inserted biosafety construct nucleic acid sequence (to be combined with a plasmid based biosafety construct) encoding a polypeptide sequence that is at least about 80%, at least about 85%, at least about 90%, at least about 95%, or at least about 99% homologous to the polypeptide sequence of SEQ ID NO: 86, 87, 88 of WO2017087580, the contents of which are herein incorporated by reference in their entirety, or a functional fragment thereof.

In some embodiments, the genetically engineered bacteria comprise a chromosome based biosafety construct nucleic acid sequence (to be combined with a plasmid based biosafety construct) that is at least about 80%, at least about 85%, at least about 90%, at least about 95%, or at least about 99% homologous to the DNA sequence of SEQ ID NO: 89, 90, 91, 92, 93, 94 of WO2017087580, the contents of which are herein incorporated by reference in their entirety or a functional fragment thereof. In some embodiments, the genetically engineered bacteria comprise a chromosome based biosafety construct nucleic acid sequence (to be combined with a plasmid based biosafety construct) encoding a polypeptide sequence that is at least about 80%, at least about 85%, at least about 90%, at least about 95%, or at least about 99% homologous to the polypeptide sequence encoded by the DNA sequence of SEQ ID NO: 89, 90, 91, 92, 93, 94 of WO2017087580, the contents of which are herein incorporated by reference in their entirety or a functional fragment thereof.

In some embodiments, the genetically engineered bacteria comprise a plasmid based biosafety construct payload nucleic acid sequence at least about 80%, at least about 85%, at least about 90%, at least about 95%, or at least about 99% homologous to the DNA sequence of SEQ ID NO: 36, 37, 74, 95, 96, 98, 99, 100, 113 of WO2017087580, the contents of which are herein incorporated by reference in their entirety or a functional fragment thereof. In some embodiments, the genetically engineered bacteria comprise a plasmid based biosafety construct payload nucleic acid sequence encoding a polypeptide which is at least about 80%, at least about 85%, at least about 90%, at least about 95%, or at least about 99% homologous to the polypeptide encoded by the DNA sequence of SEQ ID NO: 36, 37, 74, 95, 96, 98, 99, 100, 113 of WO2017087580, the contents of which are herein incorporated by reference in their entirety or a functional fragment thereof. In some embodiments, the plasmid based construct comprises one or more copies of PAL. In some embodiments, the plasmid based construct comprises one or more copies of PheP. In some embodiments, the plasmid based construct comprises one or more copies of LAAD. In some embodiments, the plasmid based construct comprises one or more copies of PAL and one or more copies of PheP. In some embodiments, the plasmid based construct comprises one or more copies of PAL and one or more copies of LAAD. In some embodiments, the plasmid based construct comprises one or more copies of LAAD and one or more copies of PheP. In some embodiments, the plasmid based construct comprises one or more copies of PAL and one or more copies of PheP and one or more copies of LAAD. In some embodiments, the phenylalanine catabolizing plasmid payload(s) (i.e., PAL, PheP, and/or LAAD) are under the control of one or more constitutive or inducible promoter(s) as described herein (e.g., low oxygen, arabinose, IPTG inducible, or a combination thereof). In some embodiments, the promoter is useful for pre-induction. In some embodiments, the promoter is useful for in vivo activation. In some embodiments, the promoter is useful for pre-induction and in vivo activity. In some embodiments, the construct comprises two or more promoters, some of which are useful for pre0induction, and some of which are useful for in vivo activity.

In some embodiments, the genetically engineered bacteria comprise a plasmid based biosafety construct nucleic acid sequence (to be combined with a chromosome based biosafety construct), e.g., that comprises a payload construct for the catabolism of phenylalanine. In some embodiments, the plasmid based construct comprises one or more copies of PAL. In some embodiments, the plasmid based construct comprises one or more copies of PheP. In some embodiments, the plasmid based construct comprises one or more copies of LAAD. In some embodiments, the plasmid based construct comprises one or more copies of PAL and one or more copies of PheP. In some embodiments, the plasmid based construct comprises one or more copies of PAL and one or more copies of LAAD. In some embodiments, the plasmid based construct comprises one or more copies of LAAD and one or more copies of PheP. In some embodiments, the plasmid based construct comprises one or more copies of PAL and one or more copies of PheP and one or more copies of LAAD. In some embodiments, the phenylalanine catabolizing plasmid payload(s) (i.e., PAL, PheP, and/or LAAD) are under the control of one or more constitutive or inducible promoter(s) as described herein (e.g., low oxygen, arabinose, IPTG inducible, or a combination thereof). In some embodiments, the promoter is useful for pre-induction. In some embodiments, the promoter is useful for in vivo activation. In some embodiments, the promoter is useful for pre-induction and in vivo activity. In some embodiments, the construct comprises two or more promoters, some of which are useful for pre0induction, and some of which are useful for in vivo activity.

In any of these embodiments, the genetically engineered bacteria comprising an auxotrophy contain one or more mutations or modifications to an endogenous phage genome. In some embodiments, the modifications to the endogenous phage genome are one or more deletion(s), insertion(s), substitution(s) or inversions(s) or combinations thereof within the phage genome. In some embodiments, the mutations are deletions. In some embodiments, the deletions comprise one or more phage genes. In some embodiments, phage genes are partially deleted. In some embodiments, the mutations are insertions. In some embodiments, the insertion comprises an antibiotic cassette as described herein. In some embodiments, one or more genes are substituted. In some embodiments, the substitution comprises an antibiotic cassette. In some embodiments, one or more phage genes are inverted. In some embodiments parts of one or more phage genes are inverted.

In some embodiments, the genetically engineered bacteria are derived from E. coli Nissle and comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the insertion comprises an antibiotic cassette. In some of the preceding embodiments, the mutation is a deletion. In some embodiments, the genetically engineered bacteria comprise one or more deletions are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 130 is deleted from the Phage 3 genome. In one embodiment, a sequence comprising SEQ ID NO: 130 is deleted from the Phage 3 genome. In one embodiment, the genetically engineered bacteria comprise modified phage genome sequence comprising SEQ ID NO: 281. In one embodiment, the genetically engineered bacteria comprise modified phage genome sequence consisting of SEQ ID NO: 281. In some embodiments, the genetically engineered bacteria further comprise one or more circuits comprising one or more gene(s) encoding one or more effector molecules. In some embodiments, the genetically engineered bacteria are capable of expressing any one or more of the circuits in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut or the tumor microenvironment, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

The addition of a Phe-auxotrophy may also have utility for increasing the rate of phenylalanine degradation. For example, the deletion of the pheA gene confers phenylalanine auxotrophy. By turning off endogenous bacterial phenylalanine production, this may drive increased uptake from the environment and also result in increased degradation of phenylalanine taken up from the environment.

### Genetic Regulatory Circuits

In some embodiments, the genetically engineered bacteria comprise multi-layered genetic regulatory circuits for expressing the constructs described herein. Suitable multi-layered genetic regulatory circuits are described in International Patent Application PCT/US2016/39434, filed on Jun. 24, 2016, published as WO2016/210378, the contents of which is herein incorporated by reference in its entirety. The genetic regulatory circuits are useful to screen for mutant bacteria that produce an anti-cancer molecule or rescue an auxotroph. In certain embodiments, the invention provides methods for selecting genetically engineered bacteria that produce one or more genes of interest.

### Host-Plasmid Mutual Dependency

In some embodiments, the genetically engineered bacteria of the invention also comprise a plasmid that has been modified to create a host-plasmid mutual dependency. In certain embodiments, the mutually dependent host-plasmid platform is an antibiotic independent plasmid system (AIPS) (Wright et al., 2015). These and other systems and platforms are described in International Patent Application PCT/US2017/013072, filed Jan. 11, 2017, published as WO2017/123675, the contents of which is herein incorporated by reference in its entirety. Kill Switch

In some embodiments, the genetically engineered bacteria of the invention also comprise a kill switch. The kill switch is intended to actively kill genetically engineered bacteria in response to external stimuli. As opposed to an auxotrophic mutation where bacteria die because they lack an essential nutrient for survival, the kill switch is triggered by a particular factor in the environment that induces the production of toxic molecules within the microbe that cause cell death. Suitable kill switches are described in PCT/US2016/039427, filed Jun. 24, 2016 and published as WO2016210373, the contents of which is herein incorporated by reference in its entirety.

### Pharmaceutical Compositions and Formulations

Pharmaceutical compositions comprising the genetically engineered bacteria of the invention may be used to treat, manage, ameliorate, and/or prevent diseases associated with hyperphenylalaninemia, e.g., PKU. Pharmaceutical compositions of the invention comprising one or more genetically engineered bacteria, alone or in combination with prophylactic agents, therapeutic agents, and/or and pharmaceutically acceptable carriers are provided. In certain embodiments, the pharmaceutical composition comprises one species, strain, or subtype of bacteria that are engineered to comprise the genetic modifications described herein. In alternate embodiments, the pharmaceutical composition comprises two or more species, strains, and/or subtypes of bacteria that are each engineered to comprise the genetic modifications described herein.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered bacteria described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release). Suitable dosage amounts for the genetically engineered bacteria may range from about 10⁵ to 10¹² bacteria, e.g., approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹¹ bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In one embodiment, the pharmaceutical composition is administered after the subject eats a meal.

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered bacteria disclosed herein may be administered topically and formulated in the form of an ointment, cream, transdermal patch, lotion, gel, shampoo, spray, aerosol, solution, emulsion, or other form well-known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered bacteria disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered bacteria are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered bacteria described herein.

In one embodiment, the genetically engineered bacteria of the disclosure may be formulated in a composition suitable for administration to pediatric subjects. As is well known in the art, children differ from adults in many aspects, including different rates of gastric emptying, pH, gastrointestinal permeability, etc. (Ivanovska et al., 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered bacteria may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. See, e.g., US 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered bacteria described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered bacteria may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of therapies of the present disclosure (see, e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

### Methods of Treatment

Another aspect of the disclosure provides methods of treating a disease comprising administering to a subject in need thereof a composition comprising an engineered bacteria disclosed herein. In some embodiments, the disclosure provides a method for treating a disease comprising administering to a subject in need thereof a composition comprising an engineered bacteria comprising a modification in an endogenous or native phage genome.

Another aspect of the disclosure provides methods of treating a disease associated with hyperphenylalaninemia or symptom(s) associated with hyperphenylalaninemia. In some embodiments, the disclosure provides a method for treating a disease associated with hyperphenylalaninemia or symptom(s) associated with hyperphenylalaninemia comprising administering to a subject in need thereof a composition comprising an engineered bacteria disclosed herein. In some embodiments, the disclosure provides a method for treating a disease associated with hyperphenylalaninemia or symptom(s) associated with hyperphenylalaninemia comprising administering to a subject in need thereof a composition comprising an engineered bacteria comprising gene sequence encoding one or PMEs, e.g., PAH and/or PAH, and/or LAAD. In some embodiments, the engineered bacteria may comprise a modification in an endogenous or native phage genome. In some embodiments, the genetically engineered bacteria further comprise one or more circuits comprising one or more gene(s) encoding one or more effector molecules (e.g. one or more PMEs, e.g., PAH and/or PAH, and/or LAAD and/or gene sequence encoding one or more Phe transporters). In some embodiments, the genetically engineered bacteria are capable of expressing any one or more of the circuits in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut or the tumor microenvironment, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits. In any of these embodiments, the modifications to the endogenous phage genome are one or more deletion(s), insertion(s), substitution(s) or inversions(s) or combinations thereof within the phage genome. In some embodiments, the mutations are deletions. In some embodiments, the deletions comprise one or more phage genes. In some embodiments, phage genes are partially deleted. In some embodiments, the mutations are insertions. In some embodiments, the insertion comprises an antibiotic cassette as described herein. IN some embodiments, one or more genes are substituted. In some embodiments, the substitution comprises an antibiotic cassette. In some embodiments, one or more phage genes are inverted. In some embodiments parts of one or more phage genes are inverted.

In some embodiments, the disclosure provides a method for treating a disease associated with hyperphenylalaninemia or symptom(s) associated with hyperphenylalaninemia comprising administering to a subject in need thereof a composition comprising an engineered bacteria comprising gene sequence encoding one or more PMEs, e.g., PAH and/or PAH, and/or LAAD and optionally gene sequence encoding one or more Phe transporters, wherein the gene sequence(s) encoding the one or more PMES are under the control of an inducible promoter, and the gene sequence encoding the one or more Phe transporters are under the control of an inducible promoter, such as any of the inducible promoters disclosed herein. The gene sequence(s) may be under the control of the same or different inducible promoters. In some embodiments, one or more of the gene sequence encoding the one or more PMEs, e.g., PAH and/or PAH, and/or LAAD are under the control of constitutive promoter. In some embodiments, one or more of the gene sequence encoding the one or more Phe transporters are under the control of constitutive promoter. In other embodiments, the bacteria may comprise one or more of the following: one or more auxotrophies, one or more kill-switches, gene guard components, and/or antibiotic resistance. In some embodiments, the insertion comprises an antibiotic cassette as described herein.

In some embodiments, the disease is selected from the group consisting of: classical or typical phenylketonuria, atypical phenylketonuria, permanent mild hyperphenylalaninemia, nonphenylketonuric hyperphenylalaninemia, phenylalanine hydroxylase deficiency, cofactor deficiency, dihydropteridine reductase deficiency, tetrahydropterin synthase deficiency, autoimmune disorders, cancer, tumors, metabolic disease (e.g., type 2 diabetes, obesity, hepatic encephalopathy, non-alcoholic fatty liver diease, and associated or related disorders), Segawa's disease, and rare disorders of metabolism. Non-limiting examples of such rare disorder of metabolism include maple sirup urine disease, isovereric acidemia, methylmalonic acidemia, propionic acidemia, hyperoxalurea, phenylketonuria, and hyperammonemia). In some embodiments, hyperphenylalaninemia is secondary to other conditions, e.g., liver diseases. In some embodiments, the invention provides methods for reducing, ameliorating, or eliminating one or more symptom(s) associated with these diseases, including but not limited to neurological deficits, mental retardation, encephalopathy, epilepsy, eczema, reduced growth, microcephaly, tremor, limb spasticity, and/or hypopigmentation. In some embodiments, the subject to be treated is a human patient.

In certain embodiments, the genetically engineered bacteria are capable of metabolizing a meabolite in the diet in order to treat a disease or disorder associated with accumulation of the metabolite (e.g., hyperphenylalaninemia, e.g., PKU). In some embodiments, the genetically engineered bacteria are delivered simultaneously with dietary protein. In other embodiments, the genetically engineered bacteria are not delivered simultaneously with dietary protein. Studies have shown that pancreatic and other glandular secretions into the intestine contain high levels of proteins, enzymes, and polypeptides, and that the amino acids produced as a result of their catabolism are reabsorbed back into the blood in a process known as “enterorecirculation” (Chang, 2007; Sarkissian et al., 1999). Thus, high intestinal levels of phenylalanine may be partially independent of food intake, and are available for breakdown by PAL. In some embodiments, the genetically engineered bacteria and dietary protein are delivered after a period of fasting or phenylalanine-restricted dieting. In these embodiments, a patient suffering from hyperphenylalaninemia may be able to resume a substantially normal diet, or a diet that is less restrictive than a phenylalanine-free diet. In some embodiments, the genetically engineered bacteria may be capable of metabolizing phenylalanine from additional sources, e.g., the blood, in order to treat a disease associated with hyperphenylalaninemia, e.g., PKU. In these embodiments, the genetically engineered bacteria need not be delivered simultaneously with dietary protein, and a phenylalanine gradient is generated, e.g., from blood to gut, and the genetically engineered bacteria metabolize phenylalanine and reduce phenylalaninemia.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria of the invention are administered orally, e.g., in a liquid suspension. In some embodiments, the genetically engineered bacteria of the invention are lyophilized in a gel cap and administered orally. In some embodiments, the genetically engineered bacteria of the invention are administered via a feeding tube or gastric shunt. In some embodiments, the genetically engineered bacteria of the invention are administered rectally, e.g., by enema. In some embodiments, the genetically engineered bacteria of the invention are administered topically, intraintestinally, intrajejunally, intraduodenally, intraileally, and/or intracolically.

In certain embodiments, the pharmaceutical composition described herein is administered to reduce levels of a certain metabolite or other type of biomarker molecule or molecule associated with or causative of the disorder in a subject. In some embodiments, the methods of the present disclosure reduce the phenylalanine levels in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the phenylalanine level in a subject before and after administration of the pharmaceutical composition. In some embodiments, the method of treating or ameliorating hyperphenylalaninemia allows one or more symptoms of the condition or disorder to improve by at least about 10%, 20%, 30%, 40%, 50%, 60%, 70%, 80%, 90%, 95%, or more.

Before, during, and after the administration of the pharmaceutical composition, levels of a certain metabolite or other type of biomarker molecule or molecule associated with or causative of the disorder (e.g. phenylalanine) in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the invention to reduce levels of a certain metabolite (e.g. phenylalanine) or other type of biomarker molecule or molecule associated with or causative of the disorder. In some embodiments, the methods may include administration of the compositions of the invention to reduce the metabolite or other type of molecule to undetectable levels in a subject. In some embodiments, the methods may include administration of the compositions of the invention to reduce concentrations of the metabolite or other type of molecule to undetectable levels, or to less than about 1%, 2%, 5%, 10%, 20%, 25%, 30%, 40%, 50%, 60%, 70%, 75%, or 80% of the subject's levels prior to treatment.

Levels of a metabolite or other type of molecule that accumulates in response to the administration of the genetically engineered bacteria upon induction of the circuitry in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods described herein may include administration of the compositions of the invention to reduce levels of a certain metabolite or other type of biomarker molecule or molecule associated with or causative of the disorder and resulting in increased levels of a metabolite or other type of molecule which accumulates as a result of the administration of the genetically engineered bacteria. In some embodiments, the methods may include administration of the compositions of the invention to reduce one metabolite or other type of molecule to undetectable levels in a subject, and concurrently and proportionately increase levels of another metabolite or other type of molecule. In some embodiments, the methods may include administration of the compositions of the invention, leading to an increase concentrations of a metabolite or other type of molecule to more than about 1%, 2%, 5%, 10%, 20%, 25%, 30%, 40%, 50%, 60%, 70%, 75%, 80%, 90%, 95%, or up to 99% or up to 100% of the subject's levels of the metabolite prior to treatment. Such increases may be measured for example in the urine, the blood, the feces, or in a tumor.

In some embodiments, the activity of genetically engineered bacteria expressing PAL (e.g., phenylalanine degrading activity) can be detected in the urine of a mammalian subject, e.g., an animal model or a human, by measuring the amounts of hippurate produced and the rate of its accumulation. Hippurate is a PAL specific breakdown product, and is normally present in human urine at low concentrations. It is the end product of metabolism of phenylalanine via the PAL pathway. Phenylalanine ammonia lyase mediates the conversion of phenylalanine to cinnamate. When cinnamate is produced in the gut, is absorbed and quickly converted to hippurate in the liver and excreted in the liver (Hoskins JA and Gray Phenylalanine ammonia lyase in the management of phenylketonuria: the relationship between ingested cinnamate and urinary hippurate in humans. J Res Commun Chem Pathol Pharmacol. 1982 February; 35(2):275-82). Phenylalanine is converted to hippurate in a 1:1 ratio, i.e., 1 mole of Phe is converted into 1 mol of hippurate. Thus, changes in urinary hippurate levels can be used as a non-invasive measure of the effect of therapies that utilize this mechanism.

Hippuric acid thus has the potential to function as a biomarker allowing monitoring of dietary adherence and treatment effect in patients receiving PAL-based regimens. It can be used as an adjunct to measurement of blood Phe levels in the management of patients and because it is a urinary biomarker, it can have advantages particularly in children to adjust protein intake—which can be challenging as needs vary based on growth.

In some embodiments, the methods of the present disclosure increase the hippurate levels in the urine of a subject by at least about 10%, 20%, 25%, 30%, 40%, 50%, 60%, 70%, 75%, 80%, 85%, 90%, 95%, 96%, 97%, 98%, 99% or more as compared to levels in an untreated or control subject. In some embodiments, the increase is measured by comparing the hippurate level in a subject before and after administration of the pharmaceutical composition of the disclosure.

In this section, the term “PAL-based drug” refers to any drug, polypeptide, biologic, or treatment regimen that has PAL activity, for example, PEG-PAL, Kuvan, a composition comprising a bacteria of the present disclosure, e.g., bacteria encoding PAL and optionally PheP transporter. In some embodiments, the disclosure provides a method for measuring PAL activity in vivo by administering to a subject, e.g., a mammalian subject, a PAL-based drug and measuring the amount of hippurate produced in the subject as a measure of PAL activity. In some embodiments, the disclosure provides a method for monitoring the therapeutic activity of a PAL-based drug by administering to a subject, e.g., a mammalian subject, the PAL-based drug and measuring the amount of hippurate produced in the subject as a measure of PAL therapeutic activity. In some embodiments, the disclosure provides a method for adjusting the dosage of a PAL-based drug by administering to a subject, e.g., a mammalian subject, the PAL-based drug, measuring the amount of hippurate produced in the subject to determine PAL activity, and adjusting (e.g., increasing or decreasing) the dosage of the drug to increase or decrease the PAL activity in the subject. In some embodiments, the disclosure provides a method for adjusting the protein intake and/or diet of a subject having hyperphenylalanemia comprising administering to the subject a PAL-based drug, measuring the amount of hippurate produced in the subject, and adjusting (e.g., increasing or decreasing) the protein intake or otherwise adjusting the diet of the subject to increase or decrease the PAL activity in the subject. In some embodiments, the disclosure provides a method for confirming adherence to a protein intake and/or diet regimen of a subject having hyperphenylalanemia comprising administering to the subject a PAL-based drug, measuring the amount of hippurate produced in the subject, and measuring PAL activity in the subject.

In some embodiments of the methods disclosed herein, both blood phenylalanine levels and urine hippurate levels are monitored in a subject. In some embodiments, blood phenylalanine and hippurate in the urine are measured at multiple time points, to determine the rate of phenylalanine breakdown. In some embodiments, hippurate levels in the urine are used evaluate PAL activity or strain activity in animal models.

In some embodiments, hippuric acid measurements in the urine, alone or in combination with blood phenylalanine measurements, are used to the strain prove mechanism of action. In some embodiments, hippuric acid measurements in the urine, alone or in combination with blood phenylalanine measurements, are used as a tool to differentiate between PAL and LAAD activity in a strain, and allow to determine the contribution of each enzyme to the overall strain activity.

In some embodiments, hippuric acid measurements in the urine, alone or in combination with blood phenylalanine measurements, are used evaluate safety in animal models and human subjects. In some embodiments, hippuric acid measurements in the urine, alone or in combination with blood phenylalanine measurements, are used in the evaluation of dose-response and optimal regimen for the desired pharmacologic effect and safety. In some embodiments, hippuric acid measurements in the urine, alone or in combination with blood phenylalanine measurements, are used as surrogate endpoint for efficacy and/or toxicity. In some embodiments, hippuric acid measurements in the urine, alone or in combination with blood phenylalanine measurements, are used to predict patients' response to a regimen comprising a therapeutic strain. In some embodiments, hippuric acid measurements in the urine, alone or in combination with blood phenylalanine measurements, are used for the identification of certain patient populations that are more likely to respond to the drug therapy. In some embodiments, hippuric acid measurements in the urine, alone or in combination with blood phenylalanine measurements, are used to avoid specific adverse events. In some embodiments, hippuric acid measurements in the urine, alone or in combination with blood phenylalanine measurements, are useful for patient selection.

In some embodiments, hippuric acid measurements in the urine, alone or in combination with blood phenylalanine measurements, are used as one method for adjusting protein intake/diet of PKU patient on a regimen which includes the administration of a therapeutic PKU strain expressing PAL.

In some embodiments, measurement of urine levels of hippuric acid, alone or in combination with blood phenylalanine measurements, is used to measure and/or monitor the activity of recombinant PAL. In some embodiments, measurement of urine levels of hippuric acid is used to measure and/or monitor the activity of recombinant pegylated PAL (Peg-PAL). In some embodiments, measurement of urine levels of hippuric acid, alone or in combination with blood phenylalanine measurements, is used to measure and/or monitor the activity of recombinant PAL administered in combination with a therapeutic strain as described herein.

In some embodiments, hippuric acid measurements in the urine, alone or in combination with blood phenylalanine measurements, are used in combination with other biomarkers, e.g., clinical safety biomarkers. In some embodiments, measurement of increases and/or decreases in certain metabolites or other types of molecules are used thein combination with measurements of other biomarkers, e.g., clinical safety biomarkers. Non-limiting examples of such safety markers include physical examination, vital signs, and electrocardiogram (ECG). Other non-limiting examples include liver safety tests known in the art, e.g., serum aspartate transaminase (AST), alanine transaminase (ALT), alkaline phosphatase (ALP), gamma-glutamyl transferase (GGT), and bilirubin. Such biosafety markers also include renal safety tests, e.g., those known in the art, e.g., blood urea nitrogen (BUN), serum creatinine, glomerular filtration rate (GFR), creatinine clearance, serum electrolytes (sodium, potassium, chloride, and bicarbonate), and complete urine analysis (color, pH, specific gravity, glucose, proteins, ketone bodies, and microscopic exam for blood, leukocytes, casts), as well as Cystatin-c, β 2-microglobulin, uric acid, clusterin, N-acetyl-beta-dglucosaminidase, neutrophil gelatinase-associated lipocalin (NGAL), N-acetyl-β-dglucosaminidase (NAG), and kidney injury molecule-1 (KIM-1). Other non-limiting examples include Hematology safety biomarkers known in the art, e.g., Complete blood count, total hemoglobin, hematocrit, red cell count, mean red cell volume, mean cell hemoglobin, red cell distribution width %, mean cell hemoglobin concentration, total white cell count, differential white cell count (Neutrophils, lymphocytes, basophils, esinophils, and monocytes), and platelets. Other no-liming examples include bone safety markers known in the art, e.g., Serum calcium and inorganic phosphates. Other non-limiting examples include basic metabolic safety biomarkers known in the art, e.g., blood glucose, triglycerides (TG), total cholesterol, low density lipoprotein cholesterol (LDLc), and high density lipoprotein cholesterol (HDL-c). Other specific safety biomarkers known in the art include, e.g., serum immunoglobulin levels, C-reactive protein (CRP), fibrinogen, thyroid stimulating hormone (TSH), thyroxine, testosterone, insulin, lactate dehydrogenase (LDH), creatine kinase (CK) and its isoenzymes, cardiac troponin (cTn), and methemoglobin.

In some embodiments, the activity of genetically engineered bacteria expressing LAAD can be specifically detected in the feces and differentiated from other E. coli strains. A Phenylalanine Deaminase Test “Phenylalanine Agar Slant” can be used for this purpose. Phenylalanine agar used to determine whether the microbe can use phenylalanine and convert it to phenyl pyruvate. When the test chemicals are added to the tube containing the sample on the phenylalanine agar, phenylpyruvate is converted to a green compound, indicating a positive test. Wild type E. coli does not produce phenylpyruvate, since they do not encode an enzyme, which can produce phenylpyruvate from phenylalanine, allowing differentiation from other E. coli strains. The genetically engineered bacteria can be differentiated from other bacterial species which are able to produce phenylpyruvate by PCR-based tests known in the art. For example, species specific sequences can be amplified. For example, universal PCR that amplifies conserved regions in various bacteria is ideal to detect any pathogen in screening of specimens. For this purpose, the conserved region of the 16S rRNA gene can be used as a target gene for the universal PCR; the 16S rRNA gene contains species-specific regions by which a large number of bacterial species can be differentiated.

In some embodiments, the Phenylalanine Deaminase Test can be used to detect the genetically engineered bacteria in a feces sample. In some embodiments, PCR-based tests can be conducted to differentiate the genetically engineered bacteria from other bacterial species.

In some embodiments, quantitative PCR (qPCR) is used to amplify, detect, and/or quantify mRNA expression levels of the gene, gene(s), or gene cassettes for producing the payloads, e.g., PME(s) and/or PheP. Primers may be designed and used to detect mRNA in a sample according to methods known in the art. In some embodiments, a fluorophore is added to a sample reaction mixture that may contain payload RNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the payload(s).

In some embodiments, quantitative PCR (qPCR) is used to amplify, detect, and/or quantify mRNA expression levels of the payload(s). Primers may be designed and used to detect mRNA in a sample according to methods known in the art. In some embodiments, a fluorophore is added to a sample reaction mixture that may contain payload, payloads, e.g., PME(s) and/or PheP and/or FNRS24Y, mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the payload(s) e.g., PME(s) and/or PheP and/or FNRS24Y.

In certain embodiments, the genetically engineered bacteria are E. coli Nissle in which endogenous or native E. coli Nissle Phage 3 has been modified as described herein. In some embodiments, Phage 3 can no longer undergo the lytic cycle due to the modification. In some embodiments, the lytic cycle is reduced or less frequent due to the modification. The genetically engineered bacteria may be destroyed, e.g., by defense factors in the gut or blood serum (Sonnenborn et al., 2009) or by activation of a kill switch, several hours or days after administration. Thus, the pharmaceutical composition comprising the genetically engineered bacteria may be re-administered at a therapeutically effective dose and frequency. Length of Nissle residence in vivo in mice is shown in FIG. 68 of WO2017087580, the contents of which are herein incorporated by reference in their entirety. In alternate embodiments, the genetically engineered bacteria are not destroyed within hours or days after administration and may propagate and colonize the gut.

The methods of the invention may comprise administration of the pharmaceutical composition alone or in combination with one or more additional therapeutic agents. In some embodiments for the treatment of hyperphenylalaninemia, the pharmaceutical composition may be administered in conjunction with the cofactor tetrahydrobiopterin (e.g., Kuvan/sapropterin), large neutral amino acids (e.g., tyrosine, tryptophan), glycomacropeptides, a probiotic (e.g., VSL3), an enzyme (e.g., pegylated-PAL), and/or other agents used in the treatment of phenylketonuria (Al Hafid and Christodoulou, 2015). See, e.g., WO2017087580 A1, the entire contents of which are incorporated by reference in its entirety.

An important consideration in the selection of the one or more additional therapeutic agents is that the agent(s) should be compatible with the genetically engineered bacteria of the invention, e.g., the agent(s) must not interfere with or kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-phenylalanine diet. The dosage of the pharmaceutical composition and the frequency of administration may be selected based on the severity of the symptoms and the progression of the disease. The appropriate therapeutically effective dose and/or frequency of administration can be selected by a treating clinician. The methods of the invention also include kits comprising the pharmaceutical composition described herein. The kit can include one or more other elements including, but not limited to: instructions for use; other reagents, e.g., a label, an additional therapeutic agent; devices or materials for measuring levels of metabolites or other types of molecules associated with the disorder, in a subject; devices or other materials for preparing the pharmaceutical composition of the invention for administration; and devices or other materials for administration to a subject. Instructions for use can include guidance for therapeutic application, such as suggested dosages and/or modes of administration, e.g., in a patient with the disorder. The kit can further contain at least one additional therapeutic agent, and/or one or more additional genetically engineered bacterial strains of the invention, formulated as appropriate, in one or more separate pharmaceutical preparations.

In some embodiments, the kit is used for administration of the pharmaceutical composition to a subject. In some embodiments, the kit is used for administration of the pharmaceutical composition, alone or in combination with one or more additional therapeutic agents, to a subject. In some embodiments, the kit is used for measuring levels of metabolites or other types of molecules in a subject before, during, or after administration of the pharmaceutical composition to the subject. In certain embodiments, the kit is used for administration and/or re-administration of the pharmaceutical composition, alone or in combination with one or more additional therapeutic agents, when levels of metabolites or other types of molecules are increased or abnormally high. In some embodiments involving hyperphenylalaninemia, a diagnostic signal of hyperphenylalaninemia is a blood phenylalanine level of at least 2 mg/dL, at least 4 mg/dL, at least 6 mg/dL, at least 8 mg/dL, at least 10 mg/dL, at least 12 mg/dL, at least 14 mg/dL, at least 16 mg/dL, at least 18 mg/dL, at least 20 mg/dL, or at least 25 mg/dL.

In some embodiments, the genetically engineered bacteria achieve a target degradation rate of about 0.15 to about 8.01 μmol/10⁹ CFUs/hr. In some embodiments, the genetically engineered bacteria achieve a target degradation rate of about 0.15 to about 2 μmol/10⁹ CFUs/hr. In some embodiments, the genetically engineered bacteria achieve a target degradation rate of about 0.6 to about 8.01 μmol/10⁹ CFUs/hr. In some embodiments, the genetically engineered bacteria achieve a target degradation rate of about 0.2 to about 2.67 μmol/10⁹ CFUs/hr.

In some embodiments, the genetically engineered bacteria achieve a target degradation rate of about 0.15 to about 0.6 μmol/10⁹ CFUs/hr. In some embodiments, the genetically engineered bacteria achieve a target degradation rate of about 0.22 to about 0.9 μmol/10⁹ CFUs/hr. In some embodiments, the genetically engineered bacteria achieve a target degradation rate of about 0.3 to about 1.21 μmol/10⁹ CFUs/hr. In some embodiments, the genetically engineered bacteria achieve a target degradation rate of about 0.54 to about 2.16 μmol/10⁹ CFUs/hr. In some embodiments, the genetically engineered bacteria achieve a target degradation rate of about 1.13 to about 4.53 mol/10⁹ CFUs/hr. In some embodiments, the genetically engineered bacteria achieve a target degradation rate of about 1.84 to about 7.38 μmol/10⁹ CFUs/hr. In some embodiments, the genetically engineered bacteria achieve a target degradation rate of about 1.61 to about 6.43 mol/10⁹ CFUs/hr. In some embodiments, the genetically engineered bacteria achieve a target degradation rate of about 2 to about 8.01 μmol/10⁹ CFUs/hr.

In some embodiments, the genetically engineered bacteria achieve a target degradation rate of about 0.1 to about 1 μmol/10⁹ CFUs/hr. In some embodiments, the genetically engineered bacteria achieve a target degradation rate of about 1 to about 2 μmol/10⁹ CFUs/hr. In some embodiments, the genetically engineered bacteria achieve a target degradation rate of about 2 to about 3 μmol/10⁹ CFUs/hr. In some embodiments, the genetically engineered bacteria achieve a target degradation rate of about 3 to about 4 μmol/10⁹ CFUs/hr. In some embodiments, the genetically engineered bacteria achieve a target degradation rate of about 4 to about 5 μmol/10⁹ CFUs/hr. In some embodiments, the genetically engineered bacteria achieve a target degradation rate of about 5 to about 6 μmol/10⁹ CFUs/hr. In some embodiments, the genetically engineered bacteria achieve a target degradation rate of about 6 to about 7 μmol/10⁹ CFUs/hr. In some embodiments, the genetically engineered bacteria achieve a target degradation rate of about 7 to about 8 mol/10⁹ CFUs/hr.

In some embodiments, the genetically engineered bacteria achieve a target reduction rate of less than 0.15 μmol/10⁹ CFUs/hr. In some embodiments, the genetically engineered bacteria achieve a target degradation rate of greater than 8.01 μmol/10⁹ CFUs/hr.

In some embodiments, the genetically engineered bacteria achieve a target reduction of between about 178 mg and 2382 mg. In some embodiments, the genetically engineered bacteria achieve a target reduction of 1.08 mmol to 14.42 mmol. In some embodiments, the reduction is less than 1.08 mmol. In some embodiments, the reduction is greater than 14.42 mmol.

In some embodiments, target reduction and target degradation rates are based on classical PKU phenylalanine levels. In some embodiments, the target reduction and target degradation rates are based on phenylalanine levels observed in mild PKU. In some embodiments, target reduction and target degradation rates are based on phenylalanine levels observed in mild hyperphenylalaninemia.

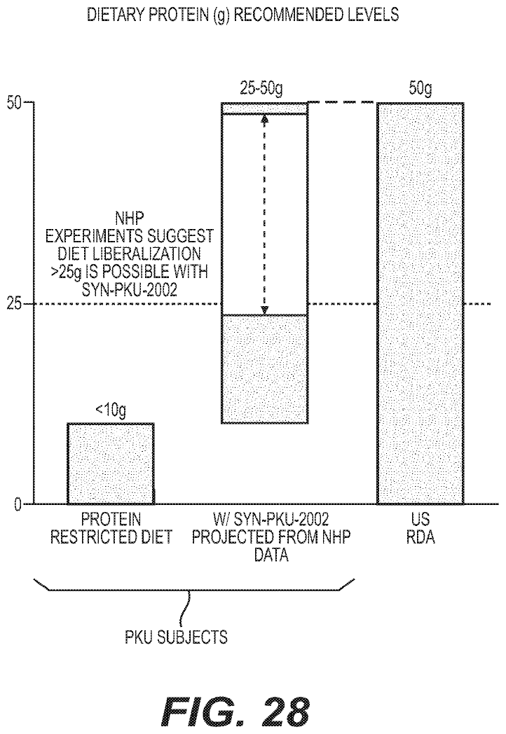

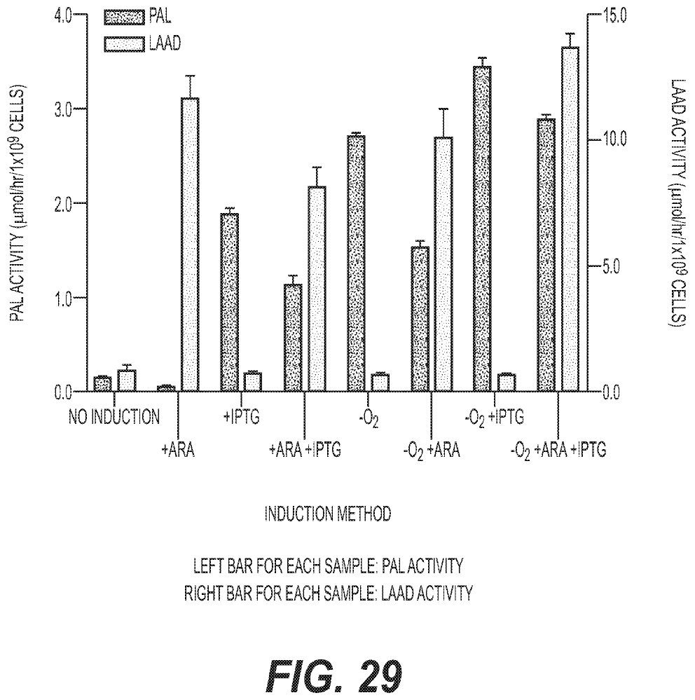

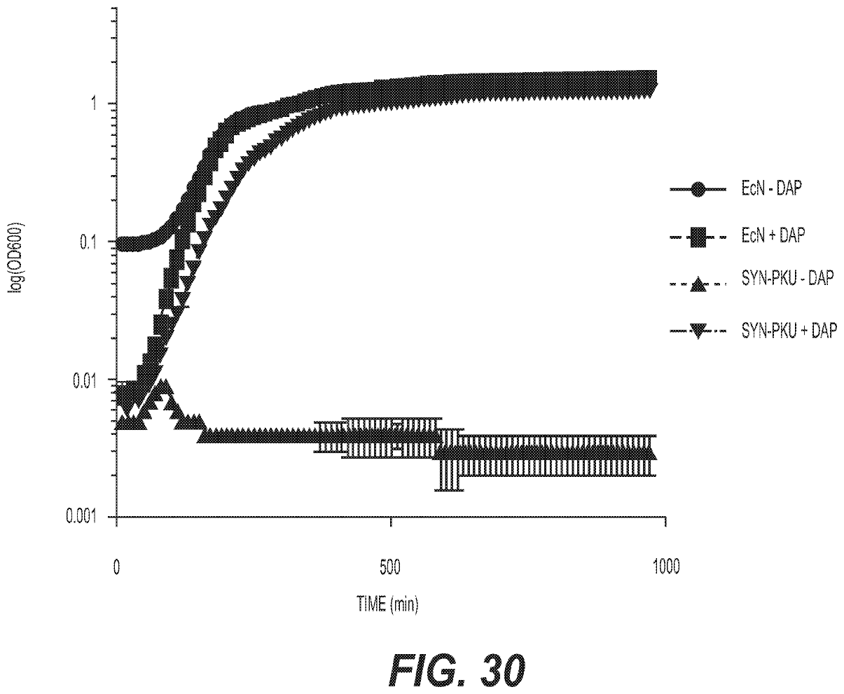

In some embodiments, administration of the genetically engineered bacteria allow for diet liberalization (partial or complete) (see FIG. 28).

### Treatment In Vivo

The genetically engineered bacteria of the invention may be evaluated in vivo, e.g., in an animal model. Any suitable animal model of the disease or condition may be used. In some embodiments, the animal model is a mouse model. See e.g. WO2017/087580 A1, the entire contents of which are incorporated by reference in its entirety.

In some embodiments, pharmacokinetics and pharmacodynamic studies may be conducted in non-human primates to determine any potential toxicities arising from administration of the genetically engineered bacteria. Non-limiting examples of such studies are described in Examples 30 and 31.

### Screening Methods

In some embodiments, of the disclosure a genetically engineered strain may be improved upon by using screening and selection methods, e.g., to increase activity of an effector (e.g. to increase PME enzymatic activity) or to increase the ability of a strain to take up a metabolite (e.g. increased ability to take up phenylalanine). In some embodiments, the screen serves to generate a bacterial strain with improved effector activity. In some embodiments, the screen serves to generate a bacterial strain which has improved ability to take up a metabolite. In some embodiments, the screen may identify a bacterial strain with both improved effector activity and enhanced substrate import. Non-limiting examples of methods of screening which can be used are described herein.

Generation of Bacterial Strains with Enhance Ability to Transport Biomolecules

In some embodiments, the ALE method can be used to identify genetically engineered bacteria with improved phenylalanine uptake.

### Specific Screen to Improve PME Activity

Screens using genetic selection are conducted to improve phenylalanine consumption in the genetically engineered bacteria. Toxic phenylalanine analogs exert their mechanism of action (MOA) by being incorporated into cellular protein, causing cell death. These compounds, such as paralog p-fluoro-DL-phenylalanine and ortholog o-fluoro-DL-phenylalanine have utility in an untargeted approach to select PAL enzymes with increased activity. Assuming that these toxic compounds can be metabolized by PAL into a non-toxic metabolite, rather than being incorporated into cellular protein, genetically engineered bacteria which have improved phenylalanine degradation activity can tolerate higher levels of these compounds and can be screened for and selected on this basis.

### REFERENCES

Al Hafid N, Christodoulou J. Phenylketonuria: a review of current and future treatments. Transl Pediatr. 2015 October; 4(4):304-317. PMID: 26835392;
Altenhoefer et al. The probiotic Escherichia coli strain Nissle 1917 interferes with invasion of human intestinal epithelial cells by different enteroinvasive bacterial pathogens. FEMS Immunol Med Microbiol. 2004 Apr. 9; 40(3):223-229. PMID: 15039098;
Andersen et al. Uracil uptake in Escherichia coli K-12: isolation of uraA mutants and cloning of the gene. J Bacteriol. 1995 April; 177(8):2008-2013. PMID: 7721693;
Arai et al. Expression of the nir and nor genes for denitrification of Pseudomonas aeruginosa requires a novel CRP/FNR-related transcriptional regulator, DNR, in addition to ANR. FEBS Lett. 1995 Aug. 28; 371(1):73-76. PMID: 7664887;
Arthur et al. Intestinal inflammation targets cancer-inducing activity of the microbiota. Science. 2012 Oct. 5; 338(6103):120-123. PMID: 22903521;
Callura et al. Tracking, Tuning and terminating microbial physiology using synthetic riboregulators. Proc Natl Acad Sci USA. 2010; 27(36):15898-15903. PMID: 20713708;
Castiglione et al. The transcription factor DNR from Pseudomonas aeruginosa specifically requires nitric oxide and haem for the activation of a target promoter in Escherichia coli. Microbiology. 2009 September; 155(Pt 9):2838-2844. PMID: 19477902;
Chang, ed. (2007) “Use of Enzyme Artificial Cells for Genetic Enzyme Defects.” In Artificial Cells: Biotechnology, Nanomedicine, Regenerative Medicine, Blood Substitutes, Bioencapsulation, and Cell/Stem Cell Therapy. World Scientific Publishing, pp. 147-159;
Clarkson et al. Diaminopimelic acid and lysine auxotrophs of Pseudomonas aeruginosa 8602. J Gen Microbiol. 1971 May; 66(2):161-169. PMID: 4999073;
Cuevas-Ramos et al. Escherichia coli induces DNA damage in vivo and triggers genomic instability in mammalian cells. Proc Natl Acad Sci USA. 2010 Jun. 22; 107(25):11537-11542. PMID: 20534522;
Danino et al. Programmable probiotics for detection of cancer in urine. Sci Transl Med. 2015 May 27; 7(289):289ra84. PMID: 26019220;
Deutscher. The mechanisms of carbon catabolite repression in bacteria. Curr Opin Microbiol. 2008 April; 11(2):87-93. PMID: 18359269;
Dinleyici et al. Saccharomyces boulardii CNCM 1-745 in different clinical conditions. Expert Opin Biol Ther. 2014 November; 14(11):1593-1609. PMID: 24995675;
Dobbelaere et al. Evaluation of nutritional status and pathophysiology of growth retardation in patients with phenylketonuria. J Inherit Metab Dis. 2003; 26(1):1-11. PMID: 12872834;
Eiglmeier et al. Molecular genetic analysis of FNR-dependent promoters. Mol Microbiol. 1989 July; 3(7):869-878. PMID: 2677602;
Estrem et al. Identification of an UP element consensus sequence for bacterial promoters. Proc Natl Acad Sci USA. 1998 Aug. 18; 95(17):9761-9766. PMID: 9707549;
Galimand et al. Positive FNR-like control of anaerobic arginine degradation and nitrate respiration in Pseudomonas aeruginosa. J Bacteriol. 1991 March; 173(5):1598-1606. PMID: 1900277;
Gardner et al. Construction of a genetic toggle switch in Escherichia coli. Nature. 2000; 403:339-342. PMID: 10659857;
Gerdes et al. Essential genes on metabolic maps. Curr Opin Biotechnol. 2006 October; 17(5):448-456. PMID: 16978855;
Gilbert et al. Molecular cloning of the phenylalanine ammonia lyase gene from Rhodosporidium toruloides in Escherichia coli K-12. J Bacteriol. 1985 January; 161(1):314-320. PMID: 2981805;
Görke B, Stulke J. Carbon catabolite repression in bacteria: many ways to make the most out of nutrients. Nat Rev Microbiol. 2008 August; 6(8):613-624. PMID: 18628769;
Hasegawa et al. Activation of a consensus FNR-dependent promoter by DNR of Pseudomonas aeruginosa in response to nitrite. FEMS Microbiol Lett. 1998 Sep. 15; 166(2):213-217. PMID: 9770276;
Hoeks et al. Adult issues in phenylketonuria. Neth J Med. 2009 January; 67(1):2-7. PMID: 19155540;
Hoeren et al. Sequence and expression of the gene encoding the respiratory nitrous-oxide reductase from Paracoccus denitrificans. Eur J Biochem. 1993 Nov. 15; 218(1):49-57. PMID: 8243476;
Hosseini et al. Propionate as a health-promoting microbial metabolite in the human gut. Nutr Rev. 2011 May; 69(5):245-258. PMID: 21521227;
Isabella et al. Deep sequencing-based analysis of the anaerobic stimulon in Neisseria gonorrhoeae. BMC Genomics. 2011 Jan. 20; 12:51. PMID: 21251255;
Ivanovska et al. Pediatric drug formulations: a review of challenges and progress. Pediatrics. 2014 August; 134(2):361-372. PMID: 25022739; Kobe et al. Regulation and crystallization of phosphorylated and dephosphorylated forms of truncated dimeric phenylalanine hydroxylase. Protein Sci. 1997 June; 6(6):1352-1357. PMID: 9194198;
Kwok et al. Nucleotide sequence of a full-length complementary DNA clone and amino acid sequence of human phenylalanine hydroxylase. Biochemistry 1985 Jan. 29; 24(3):556-561. PMID: 2986678;
Leonard J V (2006). Disorders of the urea cycle and related enzymes. Inborn Metabolic Diseases, 4^{th} ed (pp. 263-272). Springer Medizin Verlag Heidelberg;
Longo et al. Phase 1 Trial of Subcutaneous rAvPAL-PEG in Subjects with Phenylketonuria. Lancet. 2014 Jul. 5; 384(9937):37-44;
Lopez G, Anderson J C. Synthetic Auxotrophs with Ligand-Dependent Essential Genes for a BL21(DE3) Biosafety Strain. ACS Synth Biol. 2015 Dec. 18; 4(12):1279-1286. PMID: 26072987;
Macleod et al. Nutritional Management of Phenylketonuria. Ann Nestle Eng. 2010 June; 68(2):58-69. PMID: 22475869;
Meadow et al. Biosynthesis of diaminopimelic acid and lysine in Escherichia coli. Biochem J. 1959 July; 72(3):396-400. PMID: 16748796;
Miller (1972) Experiments in Molecular Genetics. Cold Spring Harbor Laboratory Press, Cold Spring Harbor, NY;
Moffitt et al. Discovery of two cyanobacterial phenylalanine ammonia lyases: kinetic and structural characterization. Biochemistry. 2007 Jan. 30; 46(4):1004-1012. PMID: 17240984;
Moore et al. Regulation of FNR dimerization by subunit charge repulsion. J Biol Chem. 2006 Nov. 3; 281(44):33268-33275. PMID: 16959764;
Nougayrede et al. Escherichia coli induces DNA double-strand breaks in eukaryotic cells. Science. 2006 Aug. 11; 313(5788):848-51. PMID: 16902142;
Olier et al. Genotoxicity of Escherichia coli Nissle 1917 strain cannot be dissociated from its probiotic activity. Gut Microbes. 2012 November-December; 3(6):501-509. PMID: 22895085;
Pi et al. Cloning and sequencing of the pheP gene, which encodes the phenylalanine-specific transport system of Escherichia coli. J Bacteriol. 1991 June; 173(12):3622-3629. PMID: 1711024;
Pi et al. Topology of the phenylalanine-specific permease of Escherichia coli. J Bacteriol. 1996 May; 178(9):2650-2655. PMID: 8626334;
Pi et al. Functional consequences of changing proline residues in the phenylalanine-specific permease of Escherichia coli. J Bacteriol. 1998 November; 180(21):5515-5519. PMID: 9791098;
Purcell et al. Towards a whole-cell modeling approach for synthetic biology. Chaos. 2013 June; 23(2):025112. PMID: 23822510;
Ray et al. The effects of mutation of the anr gene on the aerobic respiratory chain of Pseudomonas aeruginosa. FEMS Microbiol Lett. 1997 Nov. 15; 156(2):227-232. PMID: 9513270;
Reister et al. Complete genome sequence of the Gram-negative probiotic Escherichia coli strain Nissle 1917. J Biotechnol. 2014 Oct. 10; 187:106-107. PMID: 25093936;
Rembacken et al. Non-pathogenic Escherichia coli versus mesalazine for the treatment of ulcerative colitis: a randomised trial. Lancet. 1999 Aug. 21; 354(9179):635-639. PMID: 10466665;
Remington's Pharmaceutical Sciences (2012), 22^{nd} ed. Mack Publishing Co, Easton, PA.
Salmon et al. Global gene expression profiling in Escherichia coli K12. The effects of oxygen availability and FNR. J Biol Chem. 2003 Aug. 8; 278(32):29837-29855. PMID: 12754220;
Sarkissian et al. A different approach to treatment of phenylketonuria: phenylalanine degradation with recombinant phenylalanine ammonia lyase. Proc Natl Acad Sci USA. 1999 Mar. 2; 96(5):2339-2344. PMID: 10051643;
Sat et al. The Escherichia coli mazEF suicide module mediates thymineless death. J Bacteriol. 2003 March; 185(6):1803-1807. PMID: 12618443;
Sawers. Identification and molecular characterization of a transcriptional regulator from Pseudomonas aeruginosa PAO1 exhibiting structural and functional similarity to the FNR protein of Escherichia coli. Mol Microbiol. 1991 June; 5(6):1469-1481. PMID: 1787797;
Schultz. Clinical use of E. coli Nissle 1917 in inflammatory bowel disease. Inflamm Bowel Dis. 2008 July; 14(7):1012-1018. PMID: 18240278;
Sonnenborn et al. The non-pathogenic Escherichia coli strain Nissle 1917—features of a versatile probiotic. Microbial Ecology in Health and Disease. 2009; 21:122-158;
Trunk et al. Anaerobic adaptation in Pseudomonas aeruginosa: definition of the Anr and Dnr regulons. Environ Microbiol. 2010 June; 12(6):1719-1733. PMID: 20553552;
Ukena et al. Probiotic Escherichia coli Nissle 1917 inhibits leaky gut by enhancing mucosal integrity. PLoS One. 2007 Dec. 12; 2(12):e1308. PMID: 18074031;
Unden et al. Alternative respiratory pathways of Escherichia coli: energetics and transcriptional regulation in response to electron acceptors. Biochim Biophys Acta. 1997 Jul. 4; 1320(3):217-234. PMID: 9230919;
Vockley et al. Phenylalanine hydroxylase deficiency: diagnosis and management guideline. Genet Med. 2014 February; 16(2):188-200. PMID: 24385074;
Wanner et al. The phenylalanine ammonia-lyase gene family in Arabidopsis thaliana. Plant Mol Biol. 1995 January; 27(2):327-338. PMID: 7888622;
Williams et al. The gene stlA encodes a phenylalanine ammonia-lyase that is involved in the production of a stilbene antibiotic in Photorhabdus luminescens TTO1. Microbiology. 2005 August; 151(Pt 8):2543-2550. PMID: 16079333.
Winteler et al. The homologous regulators ANR of Pseudomonas aeruginosa and FNR of Escherichia coli have overlapping but distinct specificities for anaerobically inducible promoters. Microbiology. 1996 March; 142 (Pt 3):685-693. PMID: 8868444;
Wright et al. GeneGuard: A Modular Plasmid System Designed for Biosafety. ACS Synth Biol. 2015 Mar. 20; 4(3):307-316. PMID: 24847673;
Wu et al. Direct regulation of the natural competence regulator gene tfoX by cyclic AMP (cAMP) and cAMP receptor protein in Vibrios. Sci Rep. 2015 Oct. 7; 5:14921. PMID: 26442598;
Xiang L, Moore B S. Biochemical characterization of a prokaryotic phenylalanine ammonia lyase. J Bacteriol. 2005 June; 187(12):4286-4289. PMID: 15937191;
Zhang R, Lin Y. DEG 5.0, a database of essential genes in both prokaryotes and eukaryotes. Nucleic Acids Res. 2009 January; 37(Database issue):D455-D458. PMID: 18974178;
Zimmermann et al. Anaerobic growth and cyanide synthesis of Pseudomonas aeruginosa depend on anr, a regulatory gene homologous with fnr of Escherichia coli. Mol Microbiol. 1991 June; 5(6):1483-1490. PMID: 1787798.

### EXAMPLES

### Example 3. Metabolite Measurements

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Phenylalanine Quantification (Dansyl-Chloride Derivatization)

For in vitro and in vivo assays described herein, which assess the ability of the genetically engineered bacteria to degrade phenylalanine and which require quantification of phenylalanine levels in the sample, a dansyl-chloride derivatization protocol was employed as described in co-owned, pending International Patent Applications PCT/US2016/032562, filed May 13, 2016 and PCT/US2016/062369, the contents of each of which is herein incorporated by reference in its entirety.

### Trans-Cinnamic Acid Quantification (Trifluoroethylamine Derivatization)

For in vitro and in vivo assays described herein, which assess the ability of the genetically engineered bacteria to degrade phenylalanine and which require quantification of Trans-cinnamic acid levels in the sample, a trifluoroethylamine derivatization protocol was employed as as described in co-owned, pending PCT/US2016/032562, filed May 13, 2016 and PCT/US2016/062369, the contents of each of which is herein incorporated by reference in its entirety.

### Phenylalanine, Trans-Cinnamic Acid, Phenylacetic Acid, Phenylpyruvic Acid, Phenyllactic Acid, Hippuric Acid and Benzoic Acid Quantification (2-Hydrazinoquinoline Derivatization)

For in vitro and in vivo assays described herein, which assess the ability of the genetically engineered bacteria to degrade phenylalanine and which require quantification of phenylalanine, trans-cinnamic acid, phenylacetic acid, phenylpyruvic acid, phenyllactic acid, hippuric acid, and benzoic acid levels in the sample, a 2-Hydrazinoquinoline derivatization protocol was employed as as described in co-owned, pending International Patent Applications PCT/US2016/032562, filed May 13, 2016 and PCT/US2016/062369, the contents of each of which is herein incorporated by reference in its entirety.

### Hippuric Acid, Trans-Cinnamic Acid, Phenylalanine, and Phenylpyruvate Quantification in Plasma and Urine by LC-MS/MS

Hippuric acid, Trans-cinnamic acid, Phenylalanine, and Phenylpyruvate Quantification in Plasma and Urine by LC-MS/MS were measured as described in in co-owned, pending International Patent Applications PCT/US2016/032562, filed May 13, 2016 and PCT/US2016/062369, the contents of each of which is herein incorporated by reference in its entirety.

Examples 1-55 of PCT/US2016/062369, filed Nov. 16, 2016, the contents of each of which is herein incorporated by reference in its entirety describe the construction and activity of various Phe consuming strains.

### Example 27. In Vitro Activity of SYN-PKU-710

SYN-PKU-710 is an engineered bacterium derived from Escherichia coli Nissle 1917 (EcN) (ref) that has been designed to treat hyperphenylalaninemia by consuming and converting phenylalanine to the non-toxic metabolites trans-cinnamate (TCA) and phenylpyruvate (PP). It works by intercepting and degrading phenylalanine found in the intestine, which decreases the flux of phenylalanine into the blood. SYN-PKU-710 was created by a series of genetic manipulations that allowed for the degradation of phenylalanine in the microaerobic (low oxygen) environment of the human gut. The following modifications to the genome of EcN have been made to enhance phenylalanine degradation under the low oxygen conditions found in the gut while augmenting biologic containment through diaminopimelate auxotrophy:
a. Insertion of two additional copies of an endogenous Nissle gene encoding a high affinity phenylalanine transporter (PheP) under the regulatory control of an anaerobic-inducible promoter (P_{fnrS}) and the anaerobic-responsive transcriptional activator FNR
b. Insertion of three copies of a gene encoding phenylalanine ammonia lyase (PAL) derived from Photorhabdus luminescens under the regulatory control of P_{fnrS} and FNR
c. Insertion of two additional copies of the gene encoding PAL under the regulatory control of a synthetic promoter (Plac) and the lactose-responsive transcriptional repressor LacI
d. Insertion of the gene encoding L-amino acid deaminase (LAAD) derived from Proteus mirabilis under the regulatory control of the arabinose-inducible promoter (PBAD) and the arabinose-responsive transcriptional activator AraC
e. Deletion of the dapA gene that encodes 4-hydroxy-tetrahydropicolinate synthase to create a diaminopimelate auxotroph

SYN-PKU-710 was derived from EcN in a series of genetic manipulations designed to allow degradation of phenylalanine in the microaerobic (low oxygen) environment of the human gut. Genomic insertions of two additional copies of the endogenous gene encoding PheP, a high affinity phenylalanine transporter, were made within the EcN chromosome. This transporter allows the uptake of environmental phenylalanine into the bacterial cell. In addition, three copies of a gene encoding phenylalanine ammonia lyase (PAL), derived from the organism Photorhabdus luminescens, were chromosomally integrated. PAL catalyzes the conversion of phenylalanine into the non-toxic product trans-cinnamate. The genes encoding PheP and PAL were placed under the regulatory control of an anaerobic-inducible promoter (PfnrS) and the anaerobic-responsive transcriptional activator FNR. These regulatory components ensure that phenylalanine degradation machinery are produced in the anoxic environment of the human gut. Additional copies of the gene encoding PAL were inserted into the Nissle chromosome under the control of a synthetic promoter (Plac) and the LacI transcriptional repressor. These copies may be induced for expression when lactose or a lactose analog is used to alleviate transcriptional repression, and are envisioned to be utilized for the induction of phenylalanine degradation activity during the production of drug material. In this manner, initial strain potency from LacI-mediated induction will allow for immediate activity upon SYN-PKU-710 administration, while PfnrS-mediated induction will allow for de novo potency induction post-administration. A second pathway of phenylalanine degradation in SYN-PKU-710 was introduced through the chromosomal insertion of the gene encoding L-amino acid deaminase (LAAD), derived from the organism Proteus mirabilis, which converts phenylalanine into phenylpyruvate in the presence of oxygen. This gene was inserted downstream of the endogenous Nissle araC gene in a manner that allows the expression of LAAD to be induced by AraC in response to arabinose during production of drug material. The activity of PAL and LAAD have shown to be additive. The inclusion of LAAD is a mechanism to capitalize on the available oxygen expected to be found in greater abundance in the proximal GI tract.

SYN-PKU-710 was also modified with a deletion of the diaminopimelate (dapA) gene that encodes 4-hydroxy-tetrahydropicolinate synthase, which is essential for bacterial growth. This deletion renders SYN-PKU-710 unable to synthesize diaminopimelate, thereby preventing the proper formation of bacterial cell wall unless the strain is supplemented with diaminopimelate exogenously. For external manufacturing purposes, SYN-PKU-710 was also modified with a deletion of a portion of its endogenous prophage (Φ) which removes its ability to express phage particles. The strain comprising the deletion of a portion of its endogenous prophage is referred to herein as SYN-PKU-2002.

Sequences and additional details relating toe SYN-PKU-710 are described in International Patent Applications PCT/US2016/032562, filed—May 13, 2016 and PCT/US2016/062369, filed Nov. 16, 2016, the contents of each of which are herein incorporated by reference in their entireties.

To measure in vitro activity, overnight cultures were diluted 1:100 in LB and grown with shaking (250 rpm) at 37° C. After 1.5 hrs of growth, cultures were placed in a Coy anaerobic chamber supplying 90% N₂, 5% CO₂, 5% H₂. After 4 hrs of induction, bacteria were pelleted, washed in PBS, and resuspended in assay buffer (M9 minimal media with 0.5% glucose, 8.4% sodium bicarbonate, and 4 mM Phe). Rates of phenylalanine degradation (i.e., disappearance from the assay solution) or cinnamate accumulation from 30 to 90 min were normalized to 1e9 cells. Table 59 shows the normalized rates for all strains and describes genotypes and the activities of non-limiting examples of engineered plasmid-bearing strains and engineered strains comprising chromosomal insertions.

TABLE 59
Genotype and Activity of engineered plasmid-bearing strains
and engineered strains comprising chromosomal insertions.

| | | | |
|---|---|---|---|
| | | PAL Activity | LAAD activity |
| Strain Name | Genotype | (umol/hr./10\hat{ }9 cells) | (umol/hr./10\hat{ }9 cells) |

Plasmid-based strains

| | | | |
|---|---|---|---|
| SYN-PKU101 | Low copy pSC101-Ptet::PAL1, ampicillin resistant | ND | NA |
| SYN-PKU102 | High copy pColE1-Ptet::PAL1, ampicillin resistant, | ND | NA |
| SYN-PKU201 | Low copy pSC101-Ptet::PAL3, ampicillin resistant | ND | NA |
| SYN-PKU202 | High copy pColE1-Ptet::PAL3, ampicillin resistant, | ND | NA |
| SYN-PKU203 | lacZ::Ptet-pheP::cam | 0 | NA |
| SYN-PKU401 | Low copy pSC101-Ptet::PAL1, ampicillin resistant, | 1.1 | NA |
| | chromosomal lacZ::Ptet-pheP::cam | | |
| SYN-PKU402 | High copy pColE1-Ptet::PAL1, ampicillin resistant, | 0.8 | NA |
| | chromosomal lacZ::Ptet-pheP::cam | | |
| SYN-PKU302 | Low Copy pSC101-Ptet::PAL3, ampicillin resistant; | 2.2 | NA |
| | chromosomal lacZ::Ptet-pheP::cam | | |
| SYN-PKU303 | High copy pColE1-Ptet::PAL3, ampicillin resistant, | 7.1 | NA |
| | chromosomal lacZ::Ptet-pheP::cam | | |
| SYN-PKU304 | Low Copy pSC101-PfnrS-PAL3, ampicillin resistant; | 3 | NA |
| | chromosomal lacZ::PfnrS-pheP::cam | | |
| SYN-PKU305 | Low Copy pSC101-PfnrS-PAL3, kanamycin resistant; | 3 | NA |
| | chromosomal lacZ::PfnrS-pheP::cam | | |
| SYN-PKU306 | Low Copy pSC101-PfnrS-PAL3, kanamycin resistant; | 0.3 | NA |
| | thyA | | |
| SYN-PKU307 | Low Copy pSC101-PfnrS-PAL3, ampicillin resistant; | 0.3 | NA |
| SYN-PKU308 | Low Copy pSC101-PfnrS-PAL3, kanamycin resistant; | 0.3 | NA |
| SYN-PKU401 | High Copy pUC57-Ptet::LAAD; kanamycin resistant | NA | 50 (⁺O₂), |
| | | | 0 (⁻O₂) |

Integrated strains

| | | | |
|---|---|---|---|
| SYN-PKU501 | malPT:: PfnrS-PAL3::kan | 0.3 | NA |
| SYN-PKU502 | malPT:: PfnrS-PAL3::kan; bicistronic lacZ:: | ND | NA |
| | PfnrS-PAL3-pheP::cam | | |
| SYN-PKU503 | malEK::PfnrS-PAL3::cam | 0.3 | NA |
| SYN-PKU504 | agaI/rsmI::PfnrS-PAL3 | 0.3 | NA |
| SYN-PKU505 | cea::PfnrS-PAL3 | 0.3 | NA |
| SYN-PKU506 | malEK::PfnrS-PAL3::agaI/rsmI::PfnrS-PAL3; | 0.7 | NA |
| | cea::PfnrS-PAL3 | | |
| SYN-PKU507 | malEK:: PfnrS-PAL3; agaI/rsmI::PfnrS-PAL3; | 5.2 | NA |
| | cea::PfnrS-PAL3; lacZ::PfnrS-pheP::cam | | |
| SYN-PKU508 | malEK::PfnrS-PAL3; pheA auxotroph | 0.4 | NA |
| SYN-PKU509 | malEK::PfnrS-PAL3; agaI/rsmI::PfnrS-PAL3; | 4.9 | NA |
| | cea::PfnrS-PAL3; lacZ::PfnrS-pheP::cam | | |
| SYN-PKU601 | malPT::PfrnS::INT5::kan, rrnBUP -[PAL3]; | 0.9 | NA |
| | lacZ::PfnrS-pheP::cam (recombinase based strain) | | |
| SYN-PKU510 | malEK::PfnrS-PAL3; agaI/rsmI::PfnrS-PAL3; | 0.6 | NA |
| | cea::PfnrS-PAL3; | | |
| SYN-PKU511 | malEK:: PfnrS-PAL3; agaI/rsmI::PfnrS-PAL3; | 7.7 | NA |
| | cea::PfnrS-PAL3; yicS/nepI::PfnrS- | | |
| | PAL3::kan; malPT::PfnrS-PAL3; | | |
| | lacZ::PfnrS-pheP; ΔthyA | | |
| SYN-PKU204 | lacZ::PfnrS -pheP::cam | ND | NA |
| SYN-PKU512 | malEK::PfnrS-PAL3; agaI/rsmI::PfnrS- | 6.7 | NA |
| | PAL3; cea::PfnrS-PAL3; malPT::PfnrS- | | |
| | PAL3:: lacZ::PfnrS-pheP::cam; ΔthyA | | |
| SYN-PKU513 | malEK:: PfnrS-PAL3; agaI/rsmI::PfnrS- | 4.9 | NA |
| | PAL3; cea::PfnrS-PAL3; lacZ::PfnrS-pheP; | | |
| | ΔthyA | | |
| SYN-PKU514 | malEK:: PfnrS-PAL3; agaI/rsmI::PfnrS- | 0.8 | NA |
| | PAL3; cea::PfnrS-PAL3; malPT::PfnrS- | | |
| | PAL3:: ΔthyA | | |
| SYN-PKU515 | malEK:: PfnrS-PAL3; agaI/rsmI::PfnrS- | 0.7 | NA |
| | PAL3; cea::PfnrS-PAL3; ΔthyA | | |
| SYN-PKU516 | agaI/rsmI::PfnrS-PAL3::kan | 0.3 | NA |
| SYN-PKU517 | malEK:: PfnrS-PAL3::cam; malPT::PfnrS- | 2.9 | NA |
| | PAL3::kan, lacZ::PfnrS-pheP; ΔthyA | | |
| SYN-PKU518 | malEK-PfnrS-PAL3::cam; PfnrS::pheP::kan | 1.7 | NA |
| SYN-PKU519 | ParaBC-PAL3::cam; PfnrS-pheP::kan | 1.3 | NA |
| SYN-PKU520 | agaI/rsmI::PfnrS-PAL3::kan; PfnrS- | 2.0 | NA |
| | PheP::cam | | |
| SYN-PKU801 | ΔargR; thyA::cam | ND | NA |
| SYN-PKU701 | ParaBC-LAAD::cam; malEK-PfnrS-PAL3; | 2.7 | 28 (⁺O₂), |
| | malPT::PfnrS-PAL3::kan; PfnrS-pheP | | 0 (⁻O₂) |
| SYN-PKU521 | yicS/nepI::PfrnS-PAL3::kan; lacZ::PfnrS- | 2.4 | NA |
| | pheP::cam | | |
| SYN-PKU522 | cea::PfnrS-PAL3::kan; lacZ::PfnrS- | ND | NA |
| | pheP::cam | | |
| SYN-PKU523 | malPT::PfrnS-PAL3::kan; lacZ::PfnrS- | 0.5 | NA |
| | pheP::cam | | |
| SYN-PKU524 | malEK:: PfnrS-PAL3; malPT::PfnrS-PAL3; | 2.9 | NA |
| | lacZ::PfnrS-pheP | | |
| SYN-PKU702 | malEK:: PfnrS-PAL3; lacZ::PfnrS-pheP; | 1.5 | ND |
| | Para::LAAD | | |
| SYN-PKU703 | malEK:: PfnrS-PAL3; malPT::PfnrS-PAL3; | 3.1 | ND |
| | lacZ::PfnrS-pheP; agaI/rsmI::PfnrS::pheP; | | |
| | Para::LAAD | | |
| SYN-PKU704 | malEK:: PfnrS-PAL3; malPT::PfnrS-PAL3; | 3.5 | ND |
| | yicS/nepI::PfnrS-PAL3; lacZ::PfnrS-pheP; | | |
| | Para::LAAD | | |
| SYN-PKU705 | malEK:: PfnrS-PAL3; malPT::PfnrS-PAL3; | 3.7 | ND |
| | yicS/nepI::PfnrS-PAL3::kan; lacZ::PfnrS- | | |
| | pheP; agaI/rsmI::PfnrS::pheP Para::LAAD | | |
| SYN-PKU602 | malEK:: PT7::PAL3; Para::INT5::cam | 2.4 | NA |
| | (recombinase); lacZ::PfnrS-pheP; | | |
| | malPT::Pconstitutive::T7 polymerase | | |
| | (unflipped); | | |
| SYN-PKU901 | Nissle with streptomycin resistance | NA | NA |
| SYN-PKU713 | LacZ::PfnrS-PAL3::pheP | NA | NA |
| SYN-PKU706 | malEK::PfnrS-PAL3; malPT::PfnrS-PAL3; | ND | ND |
| | yicS/nepI:: PfnrS-PAL3; lacZ::PfnrS-pheP, | | |
| | agaI/rsmI::PfnrS-pheP; Para::LAAD, | | |
| | ΔthyA::cm | | |
| SYN-PKU707 | malEK:: PfnrS-PAL3; malPT::PfnrS-PAL3; | 4.0 | NA |
| | yicS/nepI:: PfnrS-PAL3; lacZ::PfnrS-pheP, | | |
| | agaI/rsmI::PfnrS-pheP; Para::FNRS24Y::cm | | |
| SYN-PKU708 | malEK:: PfnrS-PAL3; malPT::PfnrS-PAL3; | 4.0 | 44 (⁺O₂), |
| | yicS/nepI:: PfnrS-PAL3; lacZ::PfnrS-pheP, | | 0 (⁻O₂) |
| | agaI/rsmI::PfnrS-pheP; Para::FNRS24Y- | | |
| | LAAD; ΔdapA | | |
| SYN-PKU-709 | malEK:: PfnrS-PAL3; malPT::PfnrS-PAL3; | ND | NA |
| | yicS/nepI:: PfnrS-PAL3; lacZ::PfnrS-pheP, | | |
| | agaI/rsmI::PfnrS-pheP; Para-LAAD; ΔdapA | | |
| SYN-PKU-710 | malEK:: PfnrS-PAL3; malPT::PfnrS-PAL3; | 4.4 | 40 (⁺O₂), |
| | yicS/nepI:: PfnrS-PAL3; lacZ::PfnrS-pheP, | | 0 (⁻O₂) |
| | agaI/rsmI::PfnrS-pheP; Para::LAAD; | | |
| | exo/cea:: LacIPAL3; rhtC/rhtB::LacIPAL3; | | |
| | ΔdapA | | |
| SYN-PKU711 | malEK:: PfnrS-PAL3; malPT::PfnrS-PAL3; | ND | ND |
| | yicS/nepI:: PfnrS-PAL3; lacZ::PfnrS-pheP, | | |
| | agaI/rsmI::PfnrS-pheP; Para::FNRS24Y- | | |
| | LAAD; | | |
| SYN-PKU-712 | malEK:: PfnrS-PAL3; malPT::PfnrS-PAL3; | ND | ND |
| | yicS/nepI:: PfnrS-PAL3; lacZ::PfnrS-pheP, | | |
| | agaI/rsmI::PfnrS-pheP; Para::FNRS24Y; | | |
| | ΔDapA | | |
| SYN-PKU-714 | lacZ::PfnrS-pheP, agaI/rsmI::PfnrS-pheP | ND | ND |

| | | SEQ |
|---|---|---|
| | | ID |
| Description | Sequence | NO |
| FNRS24Y (bold) | TTATTCACAACCTGCCCTAAACTCGCTCGGACTCGCCCCGGTGCATT | 64 |
| driven by the | TTTTAAATACTCGCGAGAAATAGAGTTGATCGTCAAAACCGACATTG | |
| arabinose | CGACCGACGGTGGCGATAGGCATCCGGGTGGTGCTCAAAAGCAGC | |
| inducible | TTCGCCTGACTGATGCGCTGGTCCTCGCGCCAGCTTAATACGCTAAT | |
| promoter | CCCTAACTGCTGGCGGAACAAATGCGACAGACGCGACGGCGACAG | |
| (underlined) and | GCAGACATGCTGTGCGACGCTGGCGATATCAAAATTACTGTCTGCC | |
| araC in reverse | AGGTGATCGCTGATGTACTGACAAGCCTCGCGTACCCGATTATCCAT | |
| direction (italic). | CGGTGGATGGAGCGACTCGTTAATCGCTTCCATGCGCCGCAGTAAC | |
| RBS is | AATTGCTCAAGCAGATTTATCGCCAGCAATTCCGAATAGCGCCCTTC | |
| underlined and | CCCTTGTCCGGCATTAATGATTTGCCCAAACAGGTCGCTGAAATGCG | |
| bolded (see FIG. | GCTGGTGCGCTTCATCCGGGCGAAAGAAACCGGTATTGGCAAATAT | |
| 48A of | CGACGGCCAGTTAAGCCATTCATGCCAGTAGGCGCGCGGACGAAA | |
| WO2017087580) | GTAAACCCACTGGTGATACCATTCGTGAGCCTCCGGATGACGACCG | |
| | TAGTGATGAATCTCTCCAGGCGGGAACAGCAAAATATCACCCGGTC | |
| | GGCAGACAAATTCTCGTCCCTGATTTTTCACCACCCCCTGACCGCGA | |
| | ATGGTGAGATTGAGAATATAACCTTTCATTCCCAGCGGTCGGTCGAT | |
| | AAAAAAATCGAGATAACCGTTGGCCTCAATCGGCGTTAAACCCGCCA | |
| | CCAGATGGGCGTTAAACGAGTATCCCGGCAGCAGGGGATCATTTTG | |
| | CGCTTCAGCCATACTTTTCATACTCCCGCCATTCAGAGAAGAAACCA | |
| | ATTGTCCATATTGCATCAGACATTGCCGTCACTGCGTCTTTTACTG | |
| | GCTCTTCTCGCTAACCCAACCGGTAACCCCGCTTATTAAAAGCA | |
| | TTCTGTAACAAAGCGGGACCAAAGCCATGACAAAAACGCGTAA | |
| | CAAAAGTGTCTATAATCACGGCAGAAAAGTCCACATTGATTATT | |
| | TGCACGGCGTCACACTTTGCTATGCCATAGCATTTTTATCCATA | |
| | AGATTAGCGGATCCAGCCTGACGCTTTTTTTCGCAACTCTCTAC | |
| | TGTTTCTCCATACCTCTAGAAATAATTTTGTTTAACTTTAAGA | |
| | AGGAGATATACATATGATCCCGGAAAAGCGAATTATACGGC | |
| | GCATTCAGTCTGGCGGTTGTGCTATCCATTGCCAGGATTGC | |
| | TATATCAGCCAGCTTTGCATCCCGTTCACACTCAACGAACAT | |
| | GAGCTTGATCAGCTTGATAATATCATTGAGCGGAAGAAGCC | |
| | TATTCAGAAAGGCCAGACGCTGTTTAAGGCTGGAGATGAAC | |
| | TTAAATCGCTTTATGCCATCCGCTCCGGTACGATTAAAAGTT | |
| | ATACCATCACTGAGCAAGGCGACGAGCAAATCACTGGTTTC | |
| | CATTTAGCAGGCGATCTGGTGGGATTTGATGCCATCGGCAG | |
| | CGGTCATCACCCGAGTTTCGCGCAGGCGCTGGAAACCTCGA | |
| | TGGTATGTGAAATCCCGTTCGAAACGCTGGACGATTTGTCT | |
| | GGTAAAATGCCGAATCTGCGTCAGCAGATGATGCGTCTGAT | |
| | GAGCGGTGAAATCAAAGGCGATCAGGACATGATCCTGCTGT | |
| | TGTCGAAGAAAAATGCCGAGGAACGTCTGGCTGCATTCATC | |
| | TACAACCTGTCCCGTCGTTTTGCCCAACGCGGCTTCTCCCCT | |
| | CGTGAATTCCGCCTGACGATGACTCGTGGTGATATCGGTAA | |
| | CTATCTGGGCCTGACGGTTGAAACCATCAGCCGTCTGCTGG | |
| | GTCGCTTCCAGAAAAGCGGTATGCTGGCAGTCAAAGGTAAA | |
| | TACATCACTATCGAAAATAACGATGCGCTGGCCCAGCTTGC | |
| | TGGTCATACGCGTAACGTTGCCTGA | |
| FNRS24Y | ATGATCCCGGAAAAGCGAATTATACGGCGCATTCAGTCTGGCG | 65 |
| | GTTGTGCTATCCATTGCCAGGATTGCTATATCAGCCAGCTTTGC | |
| | ATCCCGTTCACACTCAACGAACATGAGCTTGATCAGCTTGATAA | |
| | TATCATTGAGCGGAAGAAGCCTATTCAGAAAGGCCAGACGCTG | |
| | TTTAAGGCTGGAGATGAACTTAAATCGCTTTATGCCATCCGCTC | |
| | CGGTACGATTAAAAGTTATACCATCACTGAGCAAGGCGACGAG | |
| | CAAATCACTGGTTTCCATTTAGCAGGCGATCTGGTGGGATTTGA | |
| | TGCCATCGGCAGCGGTCATCACCCGAGTTTCGCGCAGGCGCTGG | |
| | AAACCTCGATGGTATGTGAAATCCCGTTCGAAACGCTGGACGA | |
| | TTTGTCTGGTAAAATGCCGAATCTGCGTCAGCAGATGATGCGTC | |
| | TGATGAGCGGTGAAATCAAAGGCGATCAGGACATGATCCTGCT | |
| | GTTGTCGAAGAAAAATGCCGAGGAACGTCTGGCTGCATTCATCT | |
| | ACAACCTGTCCCGTCGTTTTGCCCAACGCGGCTTCTCCCCTCGT | |
| | GAATTCCGCCTGACGATGACTCGTGGTGATATCGGTAACTATCT | |
| | GGGCCTGACGGTTGAAACCATCAGCCGTCTGCTGGGTCGCTTCC | |
| | AGAAAAGCGGTATGCTGGCAGTCAAAGGTAAATACATCACTAT | |
| | CGAAAATAACGATGCGCTGGCCCAGCTTGCTGGTCATACGCGT | |
| | AACGTTGCCTGA | |
| AraC (reverse | TTATTCACAACCTGCCCTAAACTCGCTCGGACTCGCCCCGGTGC | 66 |
| orientation) | ATTTTTTAAATACTCGCGAGAAATAGAGTTGATCGTCAAAACCG | |
| | ACATTGCGACCGACGGTGGCGATAGGCATCCGGGTGGTGCTCA | |
| | AAAGCAGCTTCGCCTGACTGATGCGCTGGTCCTCGCGCCAGCTT | |
| | AATACGCTAATCCCTAACTGCTGGCGGAACAAATGCGACAGAC | |
| | GCGACGGCGACAGGCAGACATGCTGTGCGACGCTGGCGATATC | |
| | AAAATTACTGTCTGCCAGGTGATCGCTGATGTACTGACAAGCCT | |
| | CGCGTACCCGATTATCCATCGGTGGATGGAGCGACTCGTTAATC | |
| | GCTTCCATGCGCCGCAGTAACAATTGCTCAAGCAGATTTATCGC | |
| | CAGCAATTCCGAATAGCGCCCTTCCCCTTGTCCGGCATTAATGA | |
| | TTTGCCCAAACAGGTCGCTGAAATGCGGCTGGTGCGCTTCATCC | |
| | GGGCGAAAGAAACCGGTATTGGCAAATATCGACGGCCAGTTAA | |
| | GCCATTCATGCCAGTAGGCGCGCGGACGAAAGTAAACCCACTG | |
| | GTGATACCATTCGTGAGCCTCCGGATGACGACCGTAGTGATGA | |
| | ATCTCTCCAGGCGGGAACAGCAAAATATCACCCGGTCGGCAGA | |
| | CAAATTCTCGTCCCTGATTTTTCACCACCCCCTGACCGCGAATG | |
| | GTGAGATTGAGAATATAACCTTTCATTCCCAGCGGTCGGTCGAT | |
| | AAAAAAATCGAGATAACCGTTGGCCTCAATCGGCGTTAAACCC | |
| | GCCACCAGATGGGCGTTAAACGAGTATCCCGGCAGCAGGGGAT | |
| | CATTTTGCGCTTCAGCCATACTTTTCATACTCCCGCCATTCAGAG | |
| | AAGAAACCAATTGTCCATATTGCAT | |
| Promoter region | CAGACATTGCCGTCACTGCGTCTTTTACTGGCTCTTCTCGCTAAC | 67 |
| | CCAACCGGTAACCCCGCTTATTAAAAGCATTCTGTAACAAAGCG | |
| | GGACCAAAGCCATGACAAAAACGCGTAACAAAAGTGTCTATAA | |
| | TCACGGCAGAAAAGTCCACATTGATTATTTGCACGGCGTCACAC | |
| | TTTGCTATGCCATAGCATTTTTATCCATAAGATTAGCGGATCCA | |
| | GCCTGACGCTTTTTTTCGCAACTCTCTACTGTTTCTCCATACCTC | |
| | TAGAAATAATTTTGTTTAACTTTAAGAAGGAGATATACAT | |
| RBS and Leader | CTCTAGAAATAATTTTGTTTAACTTTAAGAAGGAGATATACAT | 68 |
| Region | | |
| FNRS24Y | MIPEKRIIRRIQSGGCAIHCQDCYISQLCIPFTLNEHELDQLDNIIERK | 69 |
| Polypeptide | KPIQKGQTLFKAGDELKSLYAIRSGTIKSYTITEQGDEQITGFHLAG | |
| | DLVGFDAIGSGHHPSFAQALETSMVCEIPFETLDDLSGKMPNLRQQ | |
| | MMRLMSGEIKGDQDMILLLSKKNAEERLAAFIYNLSRRFAQRGFS | |
| | PREFRLTMTRGDIGNYLGLTVETISRLLGRFQKSGMLAVKGKYITIE | |
| | NNDALAQLAGHTRNVA | |
| AraC polypeptide | MQYGQLVSSLNGGSMKSMAEAQNDPLLPGYSFNAHLVAGLTPIE | 70 |
| | ANGYLDFFIDRPLGMKGYILNLTIRGQGVVKNQGREFVCRPGDILL | |
| | FPPGEIHHYGRHPEAHEWYHQWVYFRPRAYWHEWLNWPSIFANT | |
| | GFFRPDEAHQPHFSDLFGQIINAGQGEGRYSELLAINLLEQLLLRRM | |
| | EAINESLHPPMDNRVREACQYISDHLADSNFDIASVAQHVCLSPSR | |
| | LSHLFRQQLGISVLSWREDQRISQAKLLLSTTRMPIATVGRNVGFD | |
| | DQLYFSRVFKKCTGASPSEFRAGCE* | |
| Wild Type FNR | ATGATCCCGGAAAAGCGAATTATACGGCGCATTCAGTCTGGCG | 71 |
| | GTTGTGCTATCCATTGCCAGGATTGCACGATCAGCCAGCTTTGC | |
| | ATCCCGTTCACACTCAACGAACATGAGCTTGATCAGCTTGATAA | |
| | TATCATTGAGCGGAAGAAGCCTATTCAGAAAGGCCAGACGCTG | |
| | TTTAAGGCTGGAGATGAACTTAAATCGCTTTATGCCATCCGCTC | |
| | CGGTACGATTAAAAGTTATACCATCACTGAGCAAGGCGACGAG | |
| | CAAATCACTGGTTTCCATTTAGCAGGCGATCTGGTGGGATTTGA | |
| | TGCCATCGGCAGCGGTCATCACCCGAGTTTCGCGCAGGCGCTGG | |
| | AAACCTCGATGGTATGTGAAATCCCGTTCGAAACGCTGGACGA | |
| | TTTGTCTGGTAAAATGCCGAATCTGCGTCAGCAGATGATGCGTC | |
| | TGATGAGCGGTGAAATCAAAGGCGATCAGGACATGATCCTGCT | |
| | GTTGTCGAAGAAAAATGCCGAGGAACGTCTGGCTGCATTCATCT | |
| | ACAACCTGTCCCGTCGTTTTGCCCAACGCGGCTTCTCCCCTCGT | |
| | GAATTCCGCCTGACGATGACTCGTGGTGATATCGGTAACTATCT | |
| | GGGCCTGACGGTTGAAACCATCAGCCGTCTGCTGGGTCGCTTCC | |
| | AGAAAAGCGGTATGCTGGCAGTCAAAGGTAAATACATCACTAT | |
| | CGAAAATAACGATGCGCTGGCCCAGCTTGCTGGTCATACGCGT | |
| | AACGTTGCCTGA | |
| Wild Type FNR | MIPEKRIIRRIQSGGCAIHCQDCSISQLCIPFTLNEHELDQLDNIIERK | 72 |
| polypeptide | KPIQKGQTLFKAGDELKSLYAIRSGTIKSYTITEQGDEQITGFHLAG | |
| | DLVGFDAIGSGHHPSFAQALETSMVCEIPFETLDDLSGKMPNLRQQ | |
| | MMRLMSGEIKGDQDMILLLSKKNAEERLAAFIYNLSRRFAQRGFS | |
| | PREFRLTMTRGDIGNYLGLTVETISRLLGRFQKSGMLAVKGKYITIE | |
| | NNDALAQLAGHTRNVA | |

TABLE 77
Biosafety System Constructs and Sequence Components

| | | SEQ ID |
|---|---|---|
| Description | Sequence | NO |
| Biosafety Plasmid | ACTCTTCCTTTTTCAATATTATTGAAGCATTTATCAGGGT | 81 |
| System Component- | TATTGTCTCATGAGCGGATACATATTTGAATGTATTTAG | |
| dap A | AAAAATAAACAAATAGGGGAATTAAAAAAAAGCCCGCT | |
| Biosafety Plasmid | CATTAGGCGGGCTACTACCTAGGCCGCGGCCGCGCGAAT | |
| System Vector | TCGAGCTCGGTACCCGGGGATCCTCTAGAGTCGACCTGC | |
| sequences, | AGGCATGCAAGCTTGCGGCCGCGTCGTGACTGGGAAAA | |
| comprising dapA, | CCCTGGCGACTAGTCTTGGACTCCTGTTGATAGATCCAG | |
| Kid Toxin and R6K | TAATGACCTCAGAACTCCATCTGGATTTGTTCAGAACGC | |
| minimal ori, and | TCGGTTGCCGCCGGGCGTTTTTTATTGGTGAGAATCCAG | |
| promoter elements | GGGTCCCCAATAATTACGATTTAAATCACAGCAAACACC | |
| driving expression | ACGTCGGCCCTATCAGCTGCGTGCTTTCTATGAGTCGTTG | |
| of these | CTGCATAACTTGACAATTAACATCCGGCTCGTAGGGTTT | |
| components, as | GTGGAGGGCCCAAGTTCACTTAAAAAGGAGATCAACAA | |
| shown in FIG. 61A | TGAAAGCAATTTTCGTACTGAAACATCTTAATCATGCTG | |
| of WO2017087580, | GGGAGGGTTTCTAATGTTCACGGGAAGTATTGTCGCGAT | |
| | TGTTACTCCGATGGATGAAAAAGGTAATGTCTGTCGGGC | |
| | TAGCTTGAAAAAACTGATTGATTATCATGTCGCCAGCGG | |
| | TACTTCGGCGATCGTTTCTGTTGGCACCACTGGCGAGTC | |
| | CGCTACCTTAAATCATGACGAACATGCTGATGTGGTGAT | |
| | GATGACGCTGGATCTGGCTGATGGGCGCATTCCGGTAAT | |
| | TGCCGGGACCGGCGCTAACGCTACTGCGGAAGCCATTAG | |
| | CCTGACGCAGCGCTTCAATGACAGTGGTATCGTCGGCTG | |
| | CCTGACGGTAACCCCTTACTACAATCGTCCGTCGCAAGA | |
| | AGGTTTGTATCAGCATTTCAAAGCCATCGCTGAGCATAC | |
| | TGACCTGCCGCAAATTCTGTATAATGTGCCGTCCCGTAC | |
| | TGGCTGCGATCTGCTCCCGGAAACGGTGGGCCGTCTGGC | |
| | GAAAGTAAAAAATATTATCGGAATCAAAGAGGCAACAG | |
| | GGAACTTAACGCGTGTAAACCAGATCAAAGAGCTGGTTT | |
| | CAGATGATTTTGTTCTGCTGAGCGGCGATGATGCGAGCG | |
| | CGCTGGACTTCATGCAATTGGGCGGTCATGGGGTTATTT | |
| | CCGTTACGGCTAACGTCGCAGCGCGTGATATGGCCCAGA | |
| | TGTGCAAACTGGCAGCAGAAGGGCATTTTGCCGAGGCA | |
| | CGCGTTATTAATCAGCGTCTGATGCCATTACACAACAAA | |
| | CTATTTGTCGAACCCAATCCAATCCCGGTGAAATGGGCA | |
| | TGTAAGGAACTGGGTCTTGTGGCGACCGATACGCTGCGC | |
| | CTGCCAATGACACCAATCACCGACAGTGGCCGTGAGAC | |
| | GGTCAGAGCGGCGCTTAAACATGCCGGTTTGCTGTAAGA | |
| | CTTTTGTCAGGTTCCTACTGTGACGACTACCACCGATAG | |
| | ACTGGAGTGTTGCTGCGAAAAAACCCCGCCGAAGCGGG | |
| | GTTTTTTGCGAGAAGTCACCACGATTGTGCTTTACACGG | |
| | AGTAGTCGGCAGTTCCTTAAGTCAGAATAGTGGACAGGC | |
| | GGCCAAGAACTTCGTTCATGATAGTCTCCGGAACCCGTT | |
| | CGAGTCGTTTTCCGCCCCGTGCTTTCATATCAATTGTCCG | |
| | GGGTTGATCGCAACGTACAACACCTGTGGTACGTATGCC | |
| | AACACCATCCAACGACACCGCAAAGCCGGCAGTGCGGG | |
| | CAAAATTGCCTCCGCTGGTTACGGGCACAACAACAGGCA | |
| | GGCGGGTCACGCGATTAAAGGCCGCCGGTGTGACAATC | |
| | AGCACCGGCCGCGTTCCCTGCTGCTCATGACCTGCGGTA | |
| | GGATCAAGCGAGACAAGCCAGATTTCCCCTCTTTCCATC | |
| | TAGTATAACTATTGTTTCTCTAGTAACATTTATTGTACAA | |
| | CACGAGCCCATTTTTGTCAAATAAATTTTAAATTATATCA | |
| | ACGTTAATAAGACGTTGTCAATAAAATTATTTTGACAAA | |
| | ATTGGCCGGCCGGCGCGCCGATCTGAAGATCAGCAGTTC | |
| | AACCTGTTGATAGTACGTACTAAGCTCTCATGTTTCACGT | |
| | ACTAAGCTCTCATGTTTAACGTACTAAGCTCTCATGTTTA | |
| | ACGAACTAAACCCTCATGGCTAACGTACTAAGCTCTCAT | |
| | GGCTAACGTACTAAGCTCTCATGTTTCACGTACTAAGCT | |
| | CTCATGTTTGAACAATAAAATTAATATAAATCAGCAACT | |
| | TAAATAGCCTCTAAGGTTTTAAGTTTTATAAGAAAAAAA | |
| | AGAATATATAAGGCTTTTAAAGCCTTTAAGGTTTAACGG | |
| | TTGTGGACAACAAGCCAGGGATGTAACGCACTGAGAAG | |
| | CCCTTAGAGCCTCTCAAAGCAATTTTGAGTGACACAGGA | |
| | ACACTTAACGGCTGACATGGGGCGCGCCCAGCTGTCTAG | |
| | GGCGGCGGATTTGTCCTACTCAGGAGAGCGTTCACCGAC | |
| | AAACAACAGATAAAACGAAAGGCCCAGTCTTTCGACTG | |
| | AGCCTTTCGTTTTATTTGATGCCT | |
| Biosafety Plasmid | ACTCTTCCTTTTTCAATATTATTGAAGCATTTATCAGGGT | 82 |
| System Component- | TATTGTCTCATGAGCGGATACATATTTGAATGTATTTAG | |
| ThyA | AAAAATAAACAAATAGGGGAATTAAAAAAAAGCCCGCT | |
| Biosafety Plasmid | CATTAGGCGGGCTACTACCTAGGCCGCGGCCGCGCGAAT | |
| System Vector | TCGAGCTCGGTACCCGGGGATCCTCTAGAGTCGACCTGC | |
| sequences, | AGGCATGCAAGCTTGCGGCCGCGTCGTGACTGGGAAAA | |
| comprising ThyA, | CCCTGGCGACTAGTCTTGGACTCCTGTTGATAGATCCAG | |
| Kid Toxin and R6K | TAATGACCTCAGAACTCCATCTGGATTTGTTCAGAACGC | |
| minimal ori, and | TCGGTTGCCGCCGGGCGTTTTTTATTGGTGAGAATCCAG | |
| promoter elements | GGGTCCCCAATAATTACGATTTAAATCACAGCAAACACC | |
| driving expression | ACGTCGGCCCTATCAGCTGCGTGCTTTCTATGAGTCGTTG | |
| of these | CTGCATAACTTGACAATTAATCATCCGGCTCGTAGGGTT | |
| components, as | TGTGGAGGGCCCAAGTTCACTTAAAAAGGAGATCAACA | |
| shown in FIG. 61B | ATGAAAGCAATTTTCGTACTGAAACATCTTAATCATGCT | |
| of WO2017087580 | GGGGAGGGTTTCTAATGAAACAGTATTTAGAACTGATGC | |
| | AAAAAGTGCTCGACGAAGGCACACAGAAAAACGACCGT | |
| | ACCGGAACCGGAACGCTTTCCATTTTTGGTCATCAGATG | |
| | CGTTTTAACCTGCAAGATGGATTCCCGCTGGTGACAACT | |
| | AAACGTTGCCACCTGCGTTCCATCATCCATGAACTGCTG | |
| | TGGTTTCTTCAGGGCGACACTAACATTGCTTATCTACAC | |
| | GAAAACAATGTCACCATCTGGGACGAATGGGCCGATGA | |
| | AAACGGCGACCTCGGGCCAGTGTATGGTAAACAGTGGC | |
| | GTGCCTGGCCAACGCCAGATGGTCGTCATATTGACCAGA | |
| | TCACTACGGTACTGAACCAGCTGAAAAACGACCCGGATT | |
| | CGCGCCGCATTATTGTTTCAGCGTGGAACGTAGGCGAAC | |
| | TGGATAAAATGGCGCTGGCACCGTGCCATGCATTCTTCC | |
| | AGTTCTATGTGGCAGACGGCAAACTCTCTTGCCAGCTTT | |
| | ATCAGCGCTCCTGTGACGTCTTCCTCGGCCTGCCGTTCAA | |
| | CATTGCCAGCTACGCGTTATTGGTGCATATGATGGCGCA | |
| | GCAGTGCGATCTGGAAGTGGGTGATTTTGTCTGGACCGG | |
| | TGGCGACACGCATCTGTACAGCAACCATATGGATCAAAC | |
| | TCATCTGCAATTAAGCCGCGAACCGCGTCCGCTGCCGAA | |
| | GTTGATTATCAAACGTAAACCCGAATCCATCTTCGACTA | |
| | CCGTTTCGAAGACTTTGAGATTGAAGGCTACGATCCGCA | |
| | TCCGGGCATTAAAGCGCCGGTGGCTATCTAAGACTTTTG | |
| | TCAGGTTCCTACTGTGACGACTACCACCGATAGACTGGA | |
| | GTGTTGCTGCGAAAAAACCCCGCCGAAGCGGGGTTTTTT | |
| | GCGAGAAGTCACCACGATTGTGCTTTACACGGAGTAGTC | |
| | GGCAGTTCCTTAAGTCAGAATAGTGGACAGGCGGCCAA | |
| | GAACTTCGTTCATGATAGTCTCCGGAACCCGTTCGAGTC | |
| | GTTTTCCGCCCCGTGCTTTCATATCAATTGTCCGGGGTTG | |
| | ATCGCAACGTACAACACCTGTGGTACGTATGCCAACACC | |
| | ATCCAACGACACCGCAAAGCCGGCAGTGCGGGCAAAAT | |
| | TGCCTCCGCTGGTTACGGGCACAACAACAGGCAGGCGG | |
| | GTCACGCGATTAAAGGCCGCCGGTGTGACAATCAGCACC | |
| | GGCCGCGTTCCCTGCTGCTCATGACCTGCGGTAGGATCA | |
| | AGCGAGACAAGCCAGATTTCCCCTCTTTCCATCTAGTAT | |
| | AACTATTGTTTCTCTAGTAACATTTATTGTACAACACGAG | |
| | CCCATTTTTGTCAAATAAATTTTAAATTATATCAACGTTA | |
| | ATAAGACGTTGTCAATAAAATTATTTTGACAAAATTGGC | |
| | CGGCCGGCGCGCCGATCTGAAGATCAGCAGTTCAACCTG | |
| | TTGATAGTACGTACTAAGCTCTCATGTTTCACGTACTAA | |
| | GCTCTCATGTTTAACGTACTAAGCTCTCATGTTTAACGAA | |
| | CTAAACCCTCATGGCTAACGTACTAAGCTCTCATGGCTA | |
| | ACGTACTAAGCTCTCATGTTTCACGTACTAAGCTCTCATG | |
| | TTTGAACAATAAAATTAATATAAATCAGCAACTTAAATA | |
| | GCCTCTAAGGTTTTAAGTTTTATAAGAAAAAAAAGAATA | |
| | TATAAGGCTTTTAAAGCCTTTAAGGTTTAACGGTTGTGG | |
| | ACAACAAGCCAGGGATGTAACGCACTGAGAAGCCCTTA | |
| | GAGCCTCTCAAAGCAATTTTGAGTGACACAGGAACACTT | |
| | AACGGCTGACATGGGGCGCGCCCAGCTGTCTAGGGCGG | |
| | CGGATTTGTCCTACTCAGGAGAGCGTTCACCGACAAACA | |
| | ACAGATAAAACGAAAGGCCCAGTCTTTCGACTGAGCCTT | |
| | TCGTTTTATTTGATGCCT | |
| Kid toxin (reverse | TTAAGTCAGAATAGTGGACAGGCGGCCAAGAACTTCGTT | 83 |
| orientation) | CATGATAGTCTCCGGAACCCGTTCGAGTCGTTTTCCGCC | |
| | CCGTGCTTTCATATCAATTGTCCGGGGTTGATCGCAACG | |
| | TACAACACCTGTGGTACGTATGCCAACACCATCCAACGA | |
| | CACCGCAAAGCCGGCAGTGCGGGCAAAATTGCCTCCGCT | |
| | GGTTACGGGCACAACAACAGGCAGGCGGGTCACGCGAT | |
| | TAAAGGCCGCCGGTGTGACAATCAGCACCGGCCGCGTTC | |
| | CCTGCTGCTCATGACCTGCGGTAGGATCAAGCGAGACAA | |
| | GCCAGATTTCCCCTCTTTCCAT | |
| dapA | ATGTTCACGGGAAGTATTGTCGCGATTGTTACTCCGATG | 84 |
| | GATGAAAAAGGTAATGTCTGTCGGGCTAGCTTGAAAAA | |
| | ACTGATTGATTATCATGTCGCCAGCGGTACTTCGGCGAT | |
| | CGTTTCTGTTGGCACCACTGGCGAGTCCGCTACCTTAAA | |
| | TCATGACGAACATGCTGATGTGGTGATGATGACGCTGGA | |
| | TCTGGCTGATGGGCGCATTCCGGTAATTGCCGGGACCGG | |
| | CGCTAACGCTACTGCGGAAGCCATTAGCCTGACGCAGCG | |
| | CTTCAATGACAGTGGTATCGTCGGCTGCCTGACGGTAAC | |
| | CCCTTACTACAATCGTCCGTCGCAAGAAGGTTTGTATCA | |
| | GCATTTCAAAGCCATCGCTGAGCATACTGACCTGCCGCA | |
| | AATTCTGTATAATGTGCCGTCCCGTACTGGCTGCGATCT | |
| | GCTCCCGGAAACGGTGGGCCGTCTGGCGAAAGTAAAAA | |
| | ATATTATCGGAATCAAAGAGGCAACAGGGAACTTAACG | |
| | CGTGTAAACCAGATCAAAGAGCTGGTTTCAGATGATTTT | |
| | GTTCTGCTGAGCGGCGATGATGCGAGCGCGCTGGACTTC | |
| | ATGCAATTGGGCGGTCATGGGGTTATTTCCGTTACGGCT | |
| | AACGTCGCAGCGCGTGATATGGCCCAGATGTGCAAACTG | |
| | GCAGCAGAAGGGCATTTTGCCGAGGCACGCGTTATTAAT | |
| | CAGCGTCTGATGCCATTACACAACAAACTATTTGTCGAA | |
| | CCCAATCCAATCCCGGTGAAATGGGCATGTAAGGAACTG | |
| | GGTCTTGTGGCGACCGATACGCTGCGCCTGCCAATGACA | |
| | CCAATCACCGACAGTGGCCGTGAGACGGTCAGAGCGGC | |
| | GCTTAAACATGCCGGTTTGCTGTAA | |
| thyA | ATGAAACAGTATTTAGAACTGATGCAAAAAGTGCTCGAC | 85 |
| | GAAGGCACACAGAAAAACGACCGTACCGGAACCGGAAC | |
| | GCTTTCCATTTTTGGTCATCAGATGCGTTTTAACCTGCAA | |
| | GATGGATTCCCGCTGGTGACAACTAAACGTTGCCACCTG | |
| | CGTTCCATCATCCATGAACTGCTGTGGTTTCTTCAGGGCG | |
| | ACACTAACATTGCTTATCTACACGAAAACAATGTCACCA | |
| | TCTGGGACGAATGGGCCGATGAAAACGGCGACCTCGGG | |
| | CCAGTGTATGGTAAACAGTGGCGTGCCTGGCCAACGCCA | |
| | GATGGTCGTCATATTGACCAGATCACTACGGTACTGAAC | |
| | CAGCTGAAAAACGACCCGGATTCGCGCCGCATTATTGTT | |
| | TCAGCGTGGAACGTAGGCGAACTGGATAAAATGGCGCT | |
| | GGCACCGTGCCATGCATTCTTCCAGTTCTATGTGGCAGA | |
| | CGGCAAACTCTCTTGCCAGCTTTATCAGCGCTCCTGTGA | |
| | CGTCTTCCTCGGCCTGCCGTTCAACATTGCCAGCTACGC | |
| | GTTATTGGTGCATATGATGGCGCAGCAGTGCGATCTGGA | |
| | AGTGGGTGATTTTGTCTGGACCGGTGGCGACACGCATCT | |
| | GTACAGCAACCATATGGATCAAACTCATCTGCAATTAAG | |
| | CCGCGAACCGCGTCCGCTGCCGAAGTTGATTATCAAACG | |
| | TAAACCCGAATCCATCTTCGACTACCGTTTCGAAGACTT | |
| | TGAGATTGAAGGCTACGATCCGCATCCGGGCATTAAAGC | |
| | GCCGGTGGCTATCTAA | |
| Kid toxin | MERGEIWLVSLDPTAGHEQQGTRPVLIVTPAAFNRVTRLPV | 86 |
| polypeptide | VVPVTSGGNFARTAGFAVSLDGVGIRTTGVVRCDQPRTID | |
| | MKARGGKRLERVPETIMNEVLGRLSTILT* | |
| dapA polypeptide | MFTGSIVAIVTPMDEKGNVCRASLKKLIDYHVASGTSAIVS | 87 |
| | VGTTGESATLNHDEHADVVMMTLDLADGRIPVIAGTGAN | |
| | ATAEAISLTQRFNDSGIVGCLTVTPYYNRPSQEGLYQHFKAI | |
| | AEHTDLPQILYNVPSRTGCDLLPETVGRLAKVKNIIGIKEAT | |
| | GNLTRVNQIKELVSDDFVLLSGDDASALDFMQLGGHGVIS | |
| | VTANVAARDMAQMCKLAAEGHFAEARVINQRLMPLHNK | |
| | LFVEPNPIPVKWACKELGLVATDTLRLPMTPITDSGRETVR | |
| | AALKHAGLL | |
| ThyA polypeptide | MKQYLELMQKVLDEGTQKNDRTGTGTLSIFGHQMRFNLQ | 88 |
| | DGFPLVTTKRCHLRSIIHELLWFLQGDTNIAYLHENNVTIW | |
| | DEWADENGDLGPVYGKQWRAWPTPDGRHIDQITTVLNQL | |
| | KNDPDSRRIIVSAWNVGELDKMALAPCHAFFQFYVADGKL | |
| | SCQLYQRSCDVFLGLPFNIASYALLVHMMAQQCDLEVGDF | |
| | VWTGGDTHLYSNHMDQTHLQLSREPRPLPKLIIKRKPESIF | |
| | DYRFEDFEIEGYDPHPGIKAPVAI* | |

### I. Example 55. Prophage Bioinformatic Analysis of Escherichia coli Nissle 1917 and Its Engineered Derivatives

### A. Synopsis

Objective: Routine testing procedures identified bacteriophage production from Escherichia coli Nissle 1917 (E. coli Nissle; E. coli Nissle) and related engineered derivatives. In an effort to determine the source of the bacteriophage, a collaborative bioinformatics assessment of the genomes of E. coli Nissle, and engineered derivatives was conducted to analyze genomic sequences of the strains for evidence of prophages, to assess any identified prophage elements for the likelihood of producing functional phage, to compare any functional phage elements with other known phage identified among bacterial genomic sequences, and to evaluate the frequency with which prophage elements are found in other sequenced Escherichia coli (E. coli) genomes.

### Experimental Procedures

### 1. Bioanalytical Methods

a. Phage Prediction

The phage prediction software (PHAST) (Zhou, et al., “PHAST: A Fast Phage Search Tool” Nucl. Acids Res. (2011) 39(suppl 2): W347-W352) was used to search for prophage within the published and publicly available E. coli Nissle genome (Genbank accession NZ_CP007799.1) (Reister et al., Complete genome sequence of the gram-negative probiotic Escherichia coli strain Nissle 1917; J Biotechnol. 2014 Oct. 10; 187:106-7), as well as within SYN001—E. coli Nissle 1917, a research cell bank produced from a single expansion of E. coli Nissle derived from DSM 6601, lot #Jul91 (DSMZ, Braunschweig, Germany), and the genome of the genetically engineered strains.

b. DNA Sequencing and Assembly

The genome sequences of the genetically engineered strains were generated by an external resource (GENEWIZ, Boston MA) using an Illumina MiSeq DNA sequencer. To examine the sequences for phage sequences, it was first necessary to assemble the genomic sequencing reads using genome assembler software (SPAdes) version 3.9.1, using default parameters (Nurk et al., Assembling single-cell genomes and mini-metagenomes from chimeric MDA products; J Comput Biol. 2013 October; 20(10):714-37). Scaffolds with lengths less than 500 nucleotides were discarded.

c. Identification of Potential Sequence Regions Specific to a Genetically Engineered Strain

In order to identify any sequences which could be potentially present in the engineered strains but not present in the original E. coli Nissle strain, the raw read data were aligned to each of three reference genomes using software for mapping low-divergent sequences against a large reference genome (BWA MEM) (Li and Durbin, Fast and accurate short read alignment with Burrows-Wheeler transform; Bioinformatics. 2009 Jul. 15; 25(14):1754-60). The three reference genomes were the published sequences and publicly available E. coli Nissle genome (Genbank accession NZ_CP007799.1) (Reister et al., Complete genome sequence of the gram-negative probiotic Escherichia coli strain Nissle 1917; J Biotechnol. 2014 Oct. 10; 187:106-7), the SYN001 (wild type E. coli Nissle) version of the E. coli Nissle genome that was sequenced by Genewiz, and the expected sequence for the genome of engineered strain which was based on the sequence of SYN001 along with changes resulting from specific engineering steps used to create the engineered strain. To focus on those sequences that did not correspond to the expected sequence of the engineered strain, the reads were aligned to each reference, and those that aligned to each reference separately according to the software Samtools (Li et al., The Sequence Alignment/Map format and SAMtools; Bioinformatics. 2009 Aug. 15; 25(16):2078-9) were discarded, and the remaining reads that did not align to each reference genome were further analyzed. The same process was used to identify unique sequences in the engineered strains.

The reads that did not map to each of the three references were assembled using the genome assembler software SPAdes version 3.9.1, with default parameters (Nurk et al., Assembling single-cell genomes and mini-metagenomes from chimeric MDA products; J Comput Biol. 2013 October; 20(10):714-37). These assembled scaffolds were used to check for the presence of phage-related sequences by comparing them against the nonredundant database in the National Center for Biotechnology Information (NCBI) using the Blast tool (Altschul et al., Basic local alignment search tool; J Mol Biol. 1990 Oct. 5; 215(3):403-10).

To verify these results, a whole-genome alignment was performed between the entire assembly of the genetically engineered strain and the publicly available CP007799.1 reference genome using genome sequence alignment software (MUMmer) (Delcher et al., Fast algorithms for large-scale genome alignment and comparison; Nucleic Acids Res. 2002 Jun. 1; 30(11):2478-83). These alignments identified unique sequences from the new assembly that did not map to the reference assembly. The unique sequences were again compared against the non-redundant database in NCBI using the Blast tool (Altschul et al., Basic local alignment search tool; J Mol Biol. 1990 Oct. 5; 215(3):403-10) to check for the presence of phage-related sequence. The approach resulted in the identification of three potential phages.

d. Search for Matches to Phage 3 in Other Genomes

To further refine the size of the Phage 3 sequence identified by the PHAST bioinformatic tool, the core region of the Phage 3 sequence was determined by aligning the 59 kilobase (kb) region of Phage 3 in E. coli Nissle to the closely-related genome of E. coli BW25113, which does not contain Phage 3. Regions at the left- and right-hand ends of the 59 kb Phage 3 sequence from E. coli Nissle matched the genomic organization of genes in E. coli BW25113 which likely corresponded to host chromosomal (non-phage) sequences; however a 43 kb region in the middle of the Phage 3 sequence from E. coli Nissle was not present in E. coli BW25113 and appeared as an insertion between two host chromosomal genes present in E. coli BW25113. Therefore, by this alignment, a 43 kb core Phage 3 region was observed which was unique to E. coli Nissle and likely contains the true limits of Phage 3. This 43 kb core Phage 3 sequence was then compared for alignment against a comprehensive set of 5691 E. coli and Shigella assemblies downloaded from NCBI using the MUMmer alignment software DElcher et al., Fast algorithms for large-scale genome alignment and comparison; Nucleic Acids Res. 2002 Jun. 1; 30(11):2478-83). To identify instances of Phage 3 outside of E. coli, the entire 43 kb core Phage 3 region was compared against the nonredundant NCBI database using the Blast tool (Altschul et al., Basic local alignment search tool; J Mol Biol. 1990 Oct. 5; 215(3):403-10).

To determine whether partial hits to Phage 3 in other genomes were part of larger phage elements, the scaffolds with partial matches to Phage 3 were extracted from the other genomes and PHAST (Zhou, et al., “PHAST: A Fast Phage Search Tool” Nucl. Acids Res. (2011) 39(suppl 2): W347-W352) was used to predict the presence of prophage within these regions.

e. Large-Scale Phage Prediction Across E. coli

To assess the frequency with which prophages of any type are found among sequenced E. coli strains, a newly published, more efficient version of PHAST (PHASTER) Arndt D., et al., PHASTER: a better, faster version of the PHAST phage search tool. Nucleic Acids Res. 44, W16-W21(2016)) was used to search for the presence of any prophage elements within a large set of E. coli genomes. Because the accuracy of phage prediction algorithms is dependent on the use of high-quality genomes, a set of 287 high-quality reference sequence (Refseq) E. coli genomes was used for this search.

### B. Results

1. Phage Content of E. coli Nissle

Three high-confidence, predicted prophage sequences were found within each version of the E. coli Nissle genome, referred herein to as Phage 1, Phage 2, and Phage 3 (FIG. 1). All three of these high-scoring phage were designated by PHAST as “intact” phage, indicating that they contain all of the major components of a phage (FIG. 1). The longest predicted phage in E. coli Nissle (Phage 3) contains a total of 68 proteins, and includes a phage tail, head, portal, terminase, lysin, capsid, and integrase, all of which appear to be intact. Phage 2 contains a total of 69 proteins, and includes phage transposase, lysis, terminase, head, portal, capsid, and tail proteins. Closer inspection of Phage 2 revealed that the int/xis gene pair have been disrupted by a mobile genetic element, and that the cI repressor has been fragmented into separate DNA-binding and sensing peptides, which would be expected to prevent induction of this phage. The shortest of the intact phages predicted in E. coli Nissle, Phage 1, contains a total of 32 proteins, and includes lysis and transposase functionality. However, the absence of many structural genes within the putative prophage element termed Phage 1 calls into question its potential to release viable phage particles. Features of Phage 1-3 are summarized in Table 84.

TABLE 84
i. Summary of Putative Phage Features

| Feature | Phage 1 | Phage 2 | Phage 3 |
|---|---|---|---|
| Nissle coordinates | 241563-260441 | 1325883-1378287 | 2023188-2082243 |
| Phast score | 110 | 150 | 150 |
| Intact designation | “intact” | “intact” | “intact” |
| (score greater or | | | |
| equal 90) | | | |
| Length | 18.8 kb, | 52.4 kb | 59 kb |
| Top hit | PHAGE_Stx2_c_1717_NC_011357 | PHAGE_Entero_lambda_NC_001416 | PH AGE_Entero_c_1_NC_019706 |
| Encoded essential | Contains a lysin and a | Contains transposase, | Contains tail, head, |
| phage genes | transposase | lysis, terminase, head, | portal, terminase, |
| | | portal, capsid, tail | lysin, capsid, |
| | | | integrase |
| Total number of | 32 | 69 | 68 |
| Proteins | | | |
| Number of proteins | 18 | 52 | 59 |
| classified as phage | | | |
| Number of proteins | 12 | 12 | 8 |
| which are | | | |
| “hypothetical” | | | |

2. Engineered Strain has the Same Phage Content as E. coli Nissle

Within the assembled genome of the engineered strain, PHAST predicted the same phage content as had been predicted in the original E. coli Nissle genome. All three of the phage that were identified as “intact” phage within E. coli Nissle were also identified within the genome of the engineered strain (Phages 1-3).

### 3. Engineered Strain Contains No Additional Prophage Elements

The vast majority of the raw sequencing reads of the engineered strain mapped to the reference genomes, and there were very few sequences that were unique to the engineered strain genome. There was no evidence for phage sequence within the small amount of unique sequence. When assembled, these unique sequences resulted in only 3 scaffolds, each with length <2 kb, and no similarity to known phage sequence. This result was confirmed by performing a whole-genome alignment of the complete engineered strain assembly to the E. coli Nissle reference genome, and identifying regions within the engineered strain which did not map to the reference. Using this procedure, two of the same short sequences were identified. None of these short sequences had any similarity to any known phage sequence. These very short sequences unique to the engineered strain likely represent spurious DNA in the samples, and not phage.

4. Phage 3 is Specific to E. coli Nissle

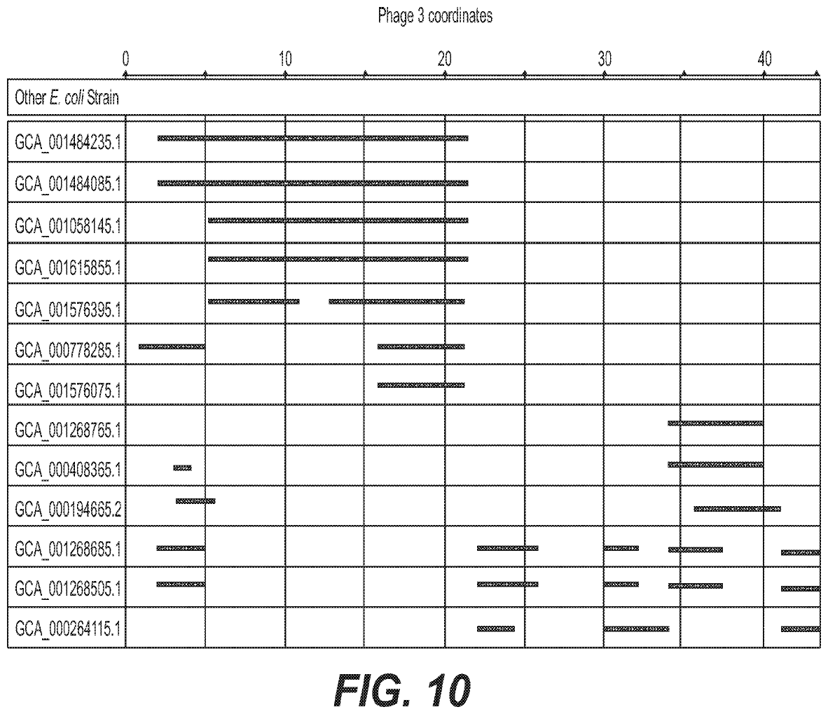

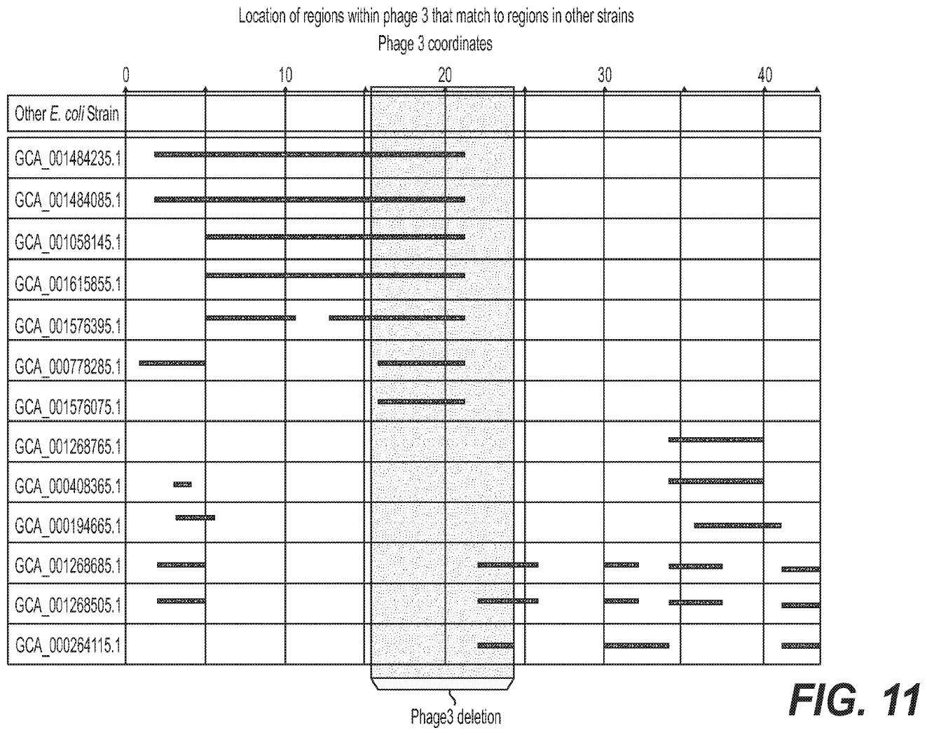

Laboratory studies have determined that the plaque-forming phage particles released by E. coli Nissle, and the engineered strain were exclusively derived from Phage 3 (SYN-17.002). Therefore, an assessment of the presence of Phage 3 (as a prophage) in other E. coli genomes was conducted. Within an extensive data set of 5691 E. coli and Shigella genome assemblies downloaded from NCBI, full-length Phage 3 was only found in E. coli Nissle. There were only two full-length, 43 kb matches covering the entire length of Phage 3 with 100% identity, and both of these corresponded to different versions of the E. coli Nissle genome (GCA_000333215.1 and GCA_000714595.1). There were five additional long partial matches (14-20 kb, with 97-99.6% identity), and four other shorter partial matches (5-10 kb, 95-98% identity) in other E. coli genomes within the data set (FIG. 10). Several other shorter fragments, split between multiple scaffolds of the same assembly, were also identified. The partial matches map to different regions of Phage 3 and are displayed in FIG. 10.

The scaffolds containing these partial matches in other genomes were extracted and used to determine that each partial match to Phage 3 comprises a fragment of a longer prophage within the other genome that had no other similarity to Phage 3. These regions were always part of a longer, high-scoring, “intact” phage prediction in the other genomes. These phage outside of E. coli Nissle corresponded to “hybrid” phage, where part of the sequence matches to Phage 3, and the rest corresponds to phage sequence which are different from Phage 3. These results indicate that the closest related phage present in E. coli share up to approximately half of their genome with the Phage 3 DNA sequence. Outside of E. coli, several partial matches were identified within more distantly related Enterobacter, 10-18 kb long, with 96-97% identity.

5. Distribution of Predicted Phage Across E. coli

Phage are very common in E. coli. Almost all of the E. coli genomes in the Refseq database contained at least one “intact”, high-scoring phage as assessed using the PHASTER phage prediction software tool, and some have up to 20 (FIG. 12). The average number of predicted phage in an E. coli genome is 6.4, which is substantially higher than the number predicted for E. coli Nissle by PHASTER. Using this newer phage prediction tool, only the two longer phage are identified in E. coli Nissle (those referred to herein as Phage 2 and Phage 3). Therefore, E. coli Nissle has substantially fewer predicted phage than most other E. coli.

### C. Conclusions

Conclusions of the bioinformatics analyses are as follows: First, E. coli Nissle and the engineered derivatives contain three candidate prophage elements, with two of the three (Phage 2 and Phage 3) containing most genetic features characteristic of intact phage genomes. Additionally, Phage 3 is unique to E. coli Nissle among a collection of almost 6000 sequenced E. coli genomes, although related sequences limited to short regions of homology with other putative prophage elements are found in a small number of genomes. Fourth, prophages are very common among E. coli strains, with E. coli Nissle containing a relatively small number as compared to the average found in a well-characterized set of sequenced E. coli genomes. These data support the conclusion that prophage presence in an engineered strain of E. coli Nissle is a consequence of the natural state of this species and the prophage features of such the engineered strains analyzed were consistent with the progenitor strain, E. coli Nissle.

### Example 56. General Protocol for the Detection and Characterization of Bacteriophage from E. coli Nissle and Genetically Engineered Derivatives

Escherichia coli Nissle 1917 (E. coli Nissle) and engineered derivatives test positive for a low level presence of phage 3 in a validated bacteriophage plaque assay. Bacteriophage plaque assays were conducted to determine presence and levels of bacteriophage. In brief, supernatants from cultures of test bacteria that were grown overnight were mixed with a phage-sensitive indicator strain and plated in soft agar to detect the formation of plaques, indicative of the presence of bacteriophage. Polymerase chain reaction (PCR) primers were designed to detect the three different endogenous prophages identified in the bioinformatics analyses, and were used to assess plaques for the presence of phage-specific DNA.

### D. Experimental Procedures

1. Phage Testing Protocol: Plaque Assay of Bacterial Virus from Escherichia coli Using Mitomycin C Induction Data Analysis

The cell lines were analyzed for the production of phage using the mitomycin C phage induction procedure (Method STM-V-708, Plaque Assay of Bacterial Virus from Escherichia coli (E. coli). Using Mitomycin C Induction, as described in Sinsheimer R L. Purification and Properties of Bacteriophage X174. J. Mol. Biol. 1959; 1:37-42, and Clowes, R C and Hayes, W. Experiments in microbial genetics. John Wiley & Sons, NY. 1968, the contents of each of which is herein incorporated by reference in its entireties). Briefly, sample (with thymidine supplemented media to support cell expansion, as appropriate) and control cells were grown overnight. A portion of the sample, positive control (E. coli, EMG 2: K (lambda), ATCC 23716, or equivalent) and negative control (E. coli, ATCC 13706, or equivalent) were removed and centrifuged, and each supernatant examined in a plaque assay for the presence of bacteriophage. Mitomycin C, at a final concentration of 2 μg/mL, was added to the remaining sample, positive and negative bacterial cultures. The cultures were then placed at 37±2° C. and shaken at 300-400 RPM until lysis occurred in the positive control (−4.5 hours). Each culture was treated with chloroform, centrifuged, and a 0.1 mL aliquot of the supernatant was examined for the presence of bacteriophage. To accomplish this, supernatants were mixed with phage-sensitive E. coli strain ATCC 13706, mixed with 0.7% agarose solution, and plated as a lawn atop lysogeny broth (LB) agar. The test was considered valid if plaques were present in the positive control and no plaques were present in the negative control.

### 2. PCR Detection of Specific Bacteriophage

a. Selection of Phage-Specific PCR Primers

Oligonucleotide polymerase chain reaction (PCR) primers were designed with specificity to each of the three putative prophage regions of E. coli Nissle, and ordered from Integrated DNA Technologies (IDT, Skokie, IL). The primers were selected after careful examination of the Nissle genome, and designed to bind completely within unique regions predicted to encode phage-specific proteins (Table 85).

TABLE 85
Phage-specific and Control Primers for
E. coli Nissle

| Primer | | Predicted | |
|---|---|---|---|
| Descrip- | | Fragment | SEQ ID |
| tion: | Primer Sequence: | Size: | NO |
| Putative | 5′-agtgcctgtacca | 366 | SEQ ID |
| Prophage 1 | gacgttc-3′ | bp | NO: 118 |
| Forward | | | |
| Putative | 5′-agaaatgacaacc | | SEQ ID |
| Prophage 1 | agagagc-3′ | | NO: 119 |
| Reverse | | | |
| Putative | 5′-ttgagtttaatat | 379 | SEQ ID |
| Prophage 2 | ggcagaac-3′ | bp | NO: 120 |
| Forward | | | |
| Putative | 5′-aaatgatcatcgc | | SEQ ID |
| Prophage 2 | gtcatc-3′ | | NO: 121 |
| Reverse | | | |
| Putative | 5′-gcatcaatcagtg | 349 | SEQ ID |
| Prophage 3 | attggc-3′ | bp | NO: 122 |
| Forward | | | |
| Putative | 5′-acgtctgaatata | | SEQ ID |
| Prophage 3 | cgggctg-3′ | | NO: 123 |
| Reverse | | | |
| Control rpoB | 5′-tccagcttgactc | 424 | SEQ ID |
| Forward | gtttcag-3′ | bp | NO: 124 |
| Control rpoB | 5′-agcaccttacccg | | SEQ ID |
| Reverse | aagagt-3′ | | NO: 125 |

Abbreviations: bp = basepair; E. coli Nissle = Escherichia coli Nissle 1917; rpoB = β subunit of bacterial RNA polymerases

An additional pair of oligonucleotide PCR primers was designed that specifically bound to rpoB, an essential bacterial-specific gene found within the E. coli Nissle genome and related strains but located outside of the three phage elements. The primer set that bound to rpoB served as a positive control for both the quality of the genomic DNA preparations, and the effectiveness of the PCR protocol. Additionally, phage DNA should not contain the rpoB gene, therefore these primers also served as a control for the purity of the phage-plaque picking technique. It was determined that while 25 PCR reaction cycles was sufficient to produce a strong band with a specific Phage PCR primer pair for plaques produced by that corresponding phage, only a weak or often no band, was observed with the rpoB primers using the same plaque DNA with 25 cycles (data not shown). For this reason, PCR analysis of plaque samples was conducted for 25 cycles.

b. PCR Reaction Conditions and Confirmation of Primer Specificity

Using the oligonucleotide primers described supra, PCR was performed against E. coli Nissle genomic DNA (gDNA) to determine whether the expected PCR products for Phage 1, 2, and 3 as well as the rpoB host genomic control were produced. The phage-negative strain ATCC 13706 served as a negative control for the PCR reaction. To prepare gDNA template for the PCR reaction, E. coli Nissle was grown in LB medium at 37±2° C. and shaken at 250 rotations per minute (rpm) overnight. One hundred (100) p L of stationary phase culture was added to a 1.5 mL microcentrifuge tube and spun at >20,000×g (15,000 rpm) in a microcentrifuge for 30 seconds. The supernatant was removed and the cell pellet was resuspended in 100 μL of sterile water. This 100 μL suspension was moved to a 0.2 mL thin wall tube and heated at 98° C. in an Eppendorf Mastercycler Pro thermocycler for 10 minutes. The resulting solution contained gDNA that was suitable for a PCR reaction. Polymerase chain reaction was performed using MyTaq™ Red Mix (Bioline) as the source of DNA nucleotides and polymerase to support DNA amplification, with E. coli Nissle gDNA as the DNA template, and mixed according to the conditions in Table 86. Polymerase chain reactions were performed in an Eppendorf Mastercycler Pro thermocycler as described in Table 87. Upon completion of PCR, 5 μL of reactions or DNA standard (1 kB⁺ ladder, Invitrogen) were loaded onto a 0.8% agarose gel for separation by electrophoresis and visualization using a Syngene ultra violet (UV) transilluminator.

TABLE 86
PCR Preparation to Amplify Putative Prophage
Regions from E. coli Nissle

| | Per Reaction | Final | |
|---|---|---|---|
| Reagent | (μL) | Concentration | |
| Nuclease-free sterile water | 23 | N/A | |
| Forward primer (100 μM) | 0.5 | 1 | μM |
| Reverse primer (100 μM) | 0.5 | 1 | μM |
| gDNA template | 1 | N/A | |
| 2X MyTaq ™ Red Mix | 25 | 0.2 | U/μL |

Abbreviation:
E. coli Nissle = Escherichia coli;
gDNA = genomic deoxyribonucleic acid;
N/A = not applicable;
PCR = polymerase chain reaction;
U = unit;
μL = microliter;
μM = micromolar

TABLE 87
PCR Reaction Cycle for Amplifying Prophage
Regions from E. coli Nissle

| Stage | Temperature (° C.) | Time (min:sec) |
|---|---|---|
| 1 | 95 | 1:00 |
| 2 | 95 | 0:15 |
| | 58 | 0:15 |
| | 72 | 0:10 |
| 3 | Repeat Stage 2: 30× | NA |
| 4 | 72 | 1:00 |
| 5 | 4 | Infinity |

Abbreviation:
° C. = degrees Celsius;
E. coli Nissle = Escherichia coli;
min = minute;
PCR = polymerase chain reaction;
sec = second(s);
NA = not applicable

### 3. Testing of Individual Plaques for Specific Phages

Bacteriophage titer assays were performed using a validated method. PCR analysis was conducted on supernatants of resulting phage titer plates that contained enumerable bacteriophage plaques (when applicable) derived from E. coli Nissle, or an engineered strain. The identity of the phage that generated the plaques was determined by PCR. Agar plugs of individual plaques were removed from the culture plates with 2 μL pipet tips. Each plug was resuspended in 0.5 mL of sterile distilled water. The plug resuspensions were vortexed for 15 seconds, then 1 μL was added to PCR reactions as the DNA template. The phage-specific PCR reactions were performed with MyTaq™ Red Mix (Bioline) as the source of DNA nucleotides and polymerase to support DNA amplification, with the plug resuspensions as the DNA template, and mixed according to the conditions in Table 5. Each set of primers from Table 85, supra was used in a separate PCR reaction to identify each of the three putative phage, with E. coli Nissle and ATCC 13706 gDNAs serving as positive and negative controls. The PCR reactions were performed according to the conditions in Table 89, and were performed for a limiting number of cycles (25 cycles) to prevent the amplification of any residual host chromosomal DNA present in the plaques. Upon completion of PCR, 5 μL of reactions or DNA standard (1 kB+ ladder, Invitrogen) were loaded onto a 0.8% agarose gel for separation by electrophoresis and visualization using a Syngene UV transilluminator.

TABLE 88
PCR Reaction Conditions to Amplify E. coli
Nissle Specific Phage from Plaque Plugs

| | Per Reaction | Final | |
|---|---|---|---|
| Reagent | (μL) | Concentration | |
| Nuclease-free sterile water | 23 | N/A | |
| Forward primer (100 μM) | 0.5 | 1 | μM |
| Reverse primer (100 μM) | 0.5 | 1 | μM |
| plaque plug or gDNA template (control) | 1 | N/A | |
| 2X MyTaq ™ Red Mix | 25 | 0.2 | U/gL |

Abbreviation:
E. coli Nissle = Escherichia coli;
gDNA = genomic deoxyribonucleic acid;
N/A = not applicable;
PCR = polymerase chain reaction;
rxn = reaction;
U = unit;
μL = microliter;
μM = micromolar

TABLE 89
PCR Reaction Cycle for Amplifying
Prophage Regions from Plaque Plugs

| | Temperature | Time |
|---|---|---|
| Stage | (° C.) | (min:sec) |
| 1 | 95 | 5:00 |
| 2 | 95 | 0:15 |
| | 58 | 0:15 |
| | 72 | 0:10 |
| 3 | Repeat Stage 2: 24× | NA |
| 4 | 72 | 1:00 |
| 5 | 4 | Infinity |

Abbreviation:
° C. = degrees Celsius;
min = minute(s);
PCR = polymerase chain reaction;
sec = second(s);
NA = not applicable

### Example 57. Testing of E. coli Nissle and Mutaflor for Presence of Phage

### 4. Summary of Phage Testing Data

Independent analyses to detect the presence of phage in several samples was conducted. The analyses showed that the Cell bank sample of E. coli Nissle from the German Collection of Microorganisms and Cell Culture (referred to herein as SYN001), and capsules of Mutaflor which contain E. coli Nissle, variably express a low level of phage under the uninduced and induced (mitomycin C treatment) conditions (Table 90).

Interestingly, E. coli Nissle strains are not always inducible as seen by the CTM lot demonstrating no phage in either condition. Additionally, even when inducible E. coli Nissle cells (Mutaflor and SYN001) express detectable phage, the levels are similar to the maximum levels seen under uninduced or induced conditions for other E. coli Nissle strains. In contrast, a positive control strain, ATCC 23716,² which is an E. coli K-12 strain that contains a bacteriophage lambda prophage, produced 5-6 orders of magnitude more phage than E. coli Nissle or its derivatives following induction. This demonstrates an obvious lack of inducibility of E. coli Nissle strains, for reasons that are not yet understood.

TABLE 90
Summary of Phage Plaque Assay Testing Results

Phage
Titer

| | Uninduced | Induced |
|---|---|---|
| Strain | (pfu/mL) | (pfu/mL) |
| E. coli Nissle - Mutaflor capsule | 0 | 2.5 × 10⁴ |
| E. coli Nissle - SYN001 | 0 | 2.0 × 10⁴ |
| ATCC 23716 (positive control) | 3.0 × 10⁴ | 3.2 × 10⁹ |

Abbreviations: CTM = clinical trial material; E. coli Nissle = Escherichia coli Nissle 1917; MCB = master cell bank; pfu = plaque forming unit; SYN001 = Escherichia coli Nissle 1917 from the German Collection of Microorganisms and Cell Cultures. Note that most strains were evaluated in separate experiments, and that the ATCC 23716 positive control strain was run as a control in each assay. The data above for ATCC23716 are the average of positive control strain for these assays.

5. Verification of Phage-Specific Primers Against E. coli Nissle Genomic DNA

To verify that the PCR analysis method could identify plaque-producing phage, PCR analysis was performed against E. coli Nissle gDNA using the primers specific for either Prophage 1, 2, 3, or the host rpoB gene (Table 85). Although not shown here, a similar analysis with the same primers against the indicator strain ATCC 13706 gave no band for the Phage primers and faint band for rpoB, consistent with this strain lacking the three E. coli Nissle prophages but containing an rpoB gene with some sequence similarity to the E. coli Nissle gene. Each primer set produced a single DNA product of the correct size as judged by agarose gel electrophoresis, which is displayed in FIG. 13. Based on this result, these primer pairs were used to assess phage DNA from individual plaques from the analyses summarized in Table 90 for the presence of the different phages.

### 6. Analysis of Individual Plaques for the Presence of Phages 1, 2, and 3

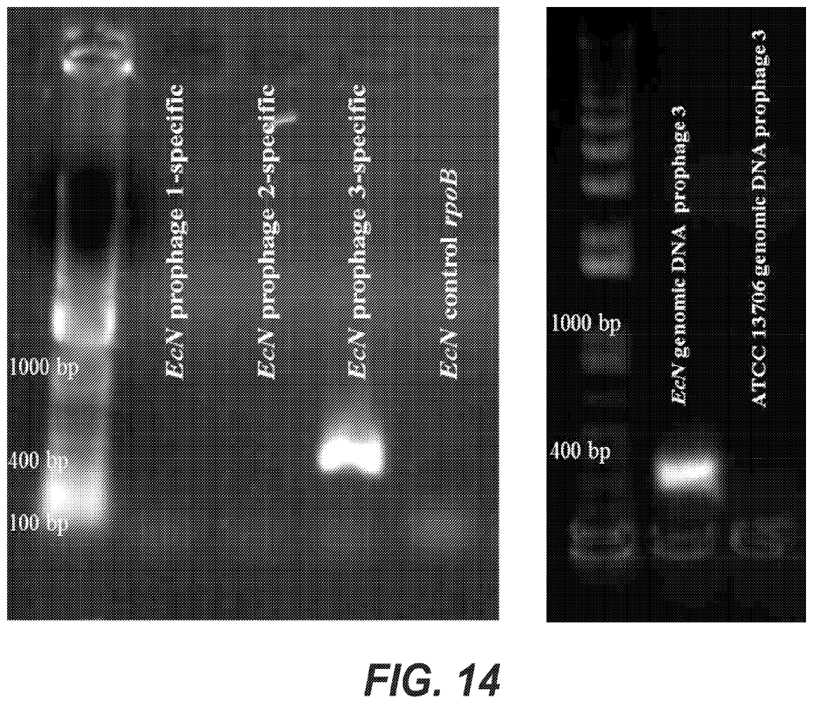

Plates with enumerable plaques from the plaque assays were analyzed by PCR analysis. Polymerase chain reaction analysis was performed against ten plaques from plates with enumerable plaques using the primers specific for either prophage 1, 2, 3, or the host rpoB gene to determine the identity of the phage that created the plaques. In some cases, plates with clearly enumerable plaques contained fewer than ten plaques, in which case all plaques were used. However, at least 6 plaques were tested for every analysis. In every PCR reaction that was run, only the primers specific for prophage 3 produced a PCR product, and this was true in all cases for each test strain and batch listed in Table 90. A representative gel analysis of this data is displayed in FIG. 14; the left panel of FIG. 14 shows a representative set of PCR reactions visualized for an individual plaque plug taken from an assay of E. coli Nissle-Mutaflor. The right panel shows a control experiment using gDNA from either E. coli Nissle or ATCC13706, the phage-negative indicator strain used in the CRL phage assay, and which does not contain a resident prophage 3. This latter finding confirms that the positive results observed in the phage plaque PCR analysis is not the result of a cross-reaction with sequences in the ATCC13706 plating strain.

### E. Conclusions

In conclusion, these data demonstrate that an expressible prophage element resides in the E. coli Nissle and Mutaflor strains evaluated. This indicates that an expressible prophage is present in E. coli Nissle. In the uninduced state, E. coli Nissle and Mutaflor produce no or low-level detectable plaques. The levels of phage produced in all cases was 5-6 orders of magnitude lower than for the positive control strain ATCC23716. Based on bioinformatics analyses of these bacterial genomes, PCR assays were developed to determine if any of the three endogenous prophage elements identified in these strains were the source of the active phage particles. The results show that the plaques derived from E. coli Nissle result from only one of the three prophage elements identified bioinformatically, referred to herein as Phage 3, as this prophage genome was uniquely amplified from plaques formed on the phage-sensitive E. coli strain ATCC 13706 (which does not natively contain the Phage 3 sequence). Taken together, these data strongly support the conclusions that Phage 3 is the responsible agent for positive phage test results, and that there appear to be no observable differences in phage production among these strains. This information also establishes that the phage produced by these strains is the result of a prophage element native to E. coli Nissle, including commercial Mutaflor capsules.

### 1. Summary of Results and Conclusions:

In conclusion, these data demonstrate that an expressible prophage element is endogenous to E. coli Nissle. The frequencies with which phage particles were produced by the tested strains, as determined by the validated plaque assay, were quantitatively at a similar low level, indicating that a generally expressible prophage is present in E. coli Nissle. Based on bioinformatics analyses of these bacterial genomes (Example 55), PCR assays were developed to determine if any of the three endogenous prophage elements identified in E. coli Nissle were the source of the plaque-forming phage particles. The results show that the plaques, when expressed from E. coli Nissle result from only one of the three prophage elements identified bioinformatically, prophage 3. This prophage genome was uniquely amplified by PCR from plaques formed on the phage-sensitive Escherichia coli (E. coli) strain ATCC 13706 (which does not natively contain the prophage 3 sequence). Taken together, these data strongly support the conclusions that prophage 3 is the responsible agent for positive phage test results, and that there appear to be no observable differences in phage production among these strains. This information also establishes that the phage produced by these strains is the result of a prophage element native to E. coli Nissle, including commercial Mutaflor.

### Example 58. Phage Testing of SYN001, SYN-PKU-710, SYN-PKU1033, and SYN-PKU1034

In this study, plaque assays on E. coli Nissle, and engineered strains comprising phenylalanine consuming circuitry, SYN-PKU-710, SYN-PKU1033 and SYN-PKU1034, were conducted to determine levels of phage produced by each strain. Table 91 provides descriptions of the strains used in this study.

TABLE 91
Strain Descriptions

| Stain Designation | Strain Description |
|---|---|
| SYN-PKU-710 | malEK:: PfnrS-PAL3; malPT::PfnrS-PAL3; yicS/nepI:: |
| | PfnrS-PAL3; lacZ::PfnrS-pheP, agaI/rsmI::PfnrS-pheP; |
| | Para::LAAD; exo/cea:: LacIPAL3; rhtC/rhtB::LacIPAL3; |
| | ΔdapA. |
| SYN-PKU1033 | Phenylalanine consuming strain which has an Antibiotic |
| | Independent Plasmid System (AIPS) comprising the |
| | plasmid and genomic components shown in FIG. 61 of |
| | WO2017087580, the contents of which are herein |
| | incorporated by reference in their entirety. The plasmid |
| | comprising the toxin is medium copy, and in lieu of the |
| | bla gene, the plasmid contains pLac pFNR PAL and PheP |
| | (IPTG and low oxygen inducible PAL and PheP in |
| | tandem). The antitoxin is integrated into dapA gene, |
| | causing a dapA auxotrophy. The strain further contains |
| | LAAD integrated into the genomic Ara locus and also has |
| | a thyA auxotrophy (Para::LAAD; ΔthyA; |
| | dapA::antitoxin); Note: SYN-PKU1034 has a weaker RBS |
| | upstream of the pheP portion of the PAL-pheP gene |
| | cassette in the AIP plasmid compared to SYN-PKU1033 |
| SYN-PKU1034 | phenylalanine consuming strain which has an AIPS |
| | comprising the plasmid and genomic components shown |
| | in FIG. 61 of WO2017087580, the contents of which are |
| | herein incorporated by reference in their entirety. The |
| | plasmid comprising the toxin is medium copy, and in lieu |
| | of the bla gene, the plasmid contains pLac pFNR PAL and |
| | PheP (IPTG and low oxygen inducible PAL and PheP in |
| | tandem). The antitoxin is integrated into dapA gene, |
| | causing a dapA auxotrophy. The strain further contains |
| | LAAD integrated into the genomic Ara locus and also has |
| | a thyA auxotrophy (Para::LAAD; ΔthyA; |
| | dapA::antitoxin); Note: SYN-PKU1034 has a weaker RBS |
| | upstream of the pheP portion of the PAL-pheP gene |
| | cassette in the AIP plasmid compared to SYN-PKU1033 |

Each strain was tested with and without mitomycin C in log phase, and without mitomycin C from stationary overnights. CFU counts were determined before supernatants were processed for the plaque assay, in order to determine the amount of plaques produced per cell.

The following test strains were grown overnight in LB (with DAP 100 ug/mL where appropriate), 3 mL in a 14 mL culture tube shaking at 250 rpm at 37 C: SYN01 (Nissle); ATC13706 (negative control); SYN-PKU-710; SYN-PKU1033, and SYN-PKU1034. An additional 20 mL culture of ATCC13706 was grown in LB in a 125 mL baffled flask (37 C, 250 rpm) for use as the sensitive plaque indicator strain. These overnight cultures were used to inoculate 10 mL cultures in LB at a 1:100 dilution (DAP 100 ug/mL where appropriate) in 125 mL baffled flasks in duplicate. For each strain, one flask contained 2 ug/mL of mitomycin C and the other culture was used as a log phase uninduced control. All flasks were grown at 37 C shaking (250 rpm) for 4.5 hours. Next, cultures and stationary uninduced overnight cultures were diluted 10-fold in PBS in a 96-well plate and plated for determination of cell counts. Ten ul of spot dilutions spanning the 10\hat{ }−3 to the 10\hat{ }−8 dilutions were plated per plate (LB plates—DAP 100 ug/mL where appropriate), in duplicate, for each strain.

After counting the cells, 1 mL from each culture (3 cultures per strain) was transferred into 1.5 mL microcentrifuge tubes and 50 μL of chloroform was added. Tubes were vortexed for 15-30 seconds, cells were spun down in a microcentrifuge for 2 minutes at maximum speed.

Supernatants were diluted in 96-well plates containing 180 μL of LB per well. 200 μL of neat supernatant for each strain was added to a first well, and 10-fold dilutions were performed with a multichannel pipette. To prepare sensitive control strain, 10 mL ATCC13706 were spun down at 4000×g in a 15 mL falcon tube, supernatant was decanted, and cells were resuspended in an equal volume of 10 mM magnesium sulfate.

Fourteen mL culture tubes containing 100 ul of ATCC13706 cell suspension were prepared for the appropriate strains and dilution of supernatants. Neat supernatant and supernatant dilutions were added to the tubes and the cell/supernatant mixture was incubated for a minimum of 5 min.

After incubation, 3 mL of liquid top agar composed of 7 g/L agar in LB media lacking yeast extract was added to tubes and the mixture was immediately poured evenly onto appropriately labelled LB plate. After plates dried, they were moved the 37C static incubator, inverted, and incubated overnight. Plaque counts are shown in Table 92.

TABLE 92
Plaque Counts

| | supernatant dilution | | | | | |
|---|---|---|---|---|---|---|
| | 0 | −1 | −2 | −3 | −4 | |
| | final dilution (for calculating pfu/mL) | | | | | cfu/mL |
| | −1 | −2 | −3 | −4 | −5 | culture |
| ATCC13706 uninduced | N | N | | | | 3.1 × 10\hat{ }9 |
| stationary | | | | | | |
| ATCC13706 uninduced log | N | N | | | | 3.2 × 10\hat{ }9 |
| ATCC13706 + MC log | N | N | | | | 2.4 × 10\hat{ }8 |
| SYN001 uninduced stationary | 4 | 1 | N | | | 2.4 × 10\hat{ }9 |
| SYN001 uninduced log | 16 | N | N | | | 4.7 × 10\hat{ }9 |
| SYN001 + MC log | TMTC | 46 | 1 | | | 0 |
| SYN-PKU-710 uninduced stationary | TMTC | 39 | 4 | N | N | 1.45 × 10\hat{ }9 |
| SYN-PKU-710 uninduced log | TMTC | 118 | 13 | N | N | 3.0 × 10\hat{ }9 |
| SYN-PKU-710 + MC log | TMTC | TMTC | 261 | 34 | 3 | 0 |
| SYN-PKU1033 uninduced | 27 | 2 | N | N | N | 7.5 × 10\hat{ }8 |
| stationary | | | | | | |
| SYN-PKU1033 uninduced log | 4 | N | N | N | N | 7.4 × 10\hat{ }8 |
| SYN-PKU1033 + MC log | 30 | 4 | 1 | N | N | 0 |
| SYN-PKU1034 uninduced | 6 | 1 | N | N | N | 1.2 × 10\hat{ }9 |
| stationary | | | | | | |
| SYN-PKU1034 uninduced log | 6 | 1 | N | N | N | 5.5 × 10\hat{ }8 |
| SYN-PKU1034 + MC log | 31 | 3 | N | N | N | 0 |

TMTC = too many to count;
N = none

Colony counting showed that 2 ug/mL of mitomycin C was completely lethal for all Nissle strains but not for ATCC13706, in which 2 ug/mL killed approximately 90% of cells.

PCR analysis was conducted to confirm that phage 3 DNA was present using primers specific to Phage 3 (not shown).

### Example 59. Deactivating Phage Production from E. coli Nissle Using Chromosomal Insertion of an Antibiotic Cassette

Bioinformatic approaches helped identify 3 regions of the genome putatively containing active phage (see Example 55). Using an in-house developed PCR method, it was shown that the active phage originated from a genomic locus between bases 2,035,867 and 2,079,177 of the E. coli Nissle genome.

### F. Knock-Out Primer Design

To inactivate the phage, lambda red recombineering was used to make a 9,687 base pair deletion. Lambda red recombineering is a procedure using recombination enzymes from a bacteriophage lambda to insert a piece of custom DNA into the chromosome of E. coli directed by flanking homology. First, primers were designed and synthesized to amplify a chloramphenicol acetyltransferase (CAT) gene flanked by flippase recognition sites (FRT) from the plasmid pKD3 (Table 93). When introduced into Nissle, this cassette provides resistance to the antibiotic chloramphenicol. In addition, these primers contain 60 base pairs of homology to the genome which directs the antibiotic cassette into the Phage loci. The Phage3 KO FWD and Phage3 KO REV primers were used to PCR amplify a 1178 base pair linear DNA fragment, which was PCR purified. The resulting DNA template was used in recombineering.

TABLE 93
Primers used in the inactivation of Phage3

| Primer | | SEQ ID |
|---|---|---|
| Name | Sequence | NO |
| Phage3 KO | TGGAGGCTTTAAGAAATACCTCGATGTG | SEQ ID |
| FWD | AACAACCGCCTGCCACGAATCTTCGTCA | NO: 126 |
| | AGCGATTACACGTCTTGAGCGAT | |
| Phage3 KO | GATAATGGTGAGATTATCCCCGGTTATA | SEQ ID |
| REV | CCGGACTTATCGCCTATTCAGAATCACT | NO: 127 |
| | GGATCTGACATGGGAATTAGCCA | |

Bold = Homology to Nissle genome; Italicized = Homology to pKD3

### G. Introduction of Lambda Red System

To prepare a strain of Nissle for deletion of phage, first the lambda red system was introduced, by transforming a pKD46 plasmid into the E. coli Nissle host strain. E. coli Nissle cells were grown overnight in LB media. The overnight culture was diluted 1:100 in 5 mL of LB media and grown until it reached an OD₆₀₀ of 0.4-0.6. All tubes, solutions, and cuvettes were pre-chilled to 4° C. The E. coli cells were centrifuged at 2,000 rpm for 5 min. at 4° C., the supernatant was removed, and the cells were resuspended in 1 mL of 4° C. water. The E. coli cells were centrifuged at 2,000 rpm for 5 min. at 4° C., the supernatant was removed, and the cells were resuspended in 0.5 mL of 4° C. water. The E. coli cells were centrifuged at 2,000 rpm for 5 min. at 4° C., the supernatant is removed, and the cells are resuspended in 0.1 mL of 4° C. water. The electroporator was set to 2.5 kV. One ng of pKD46 plasmid DNA was added to the E. coli cells, mixed by pipetting, and pipetted into a sterile, chilled cuvette. The dry cuvette was placed into the sample chamber, and the electric pulse was applied. One mL of room-temperature SOC media was immediately added, and the mixture was transferred to a culture tube and incubated at 30° C. for 1 hr. The cells were spread out on a selective media plate and incubated overnight at 30° C.

### H. Lambda Red Inactivation of Phage in Nissle

The recombineering construct was transformed into E. coli Nissle comprising pKD46 to delete the phage sequence. All tubes, solutions, and cuvettes were pre-chilled to 4° C. An overnight culture was diluted 1:100 in 5 mL of LB media containing carbenicillin and grown until it reached an OD600 of 0.1. Next, 0.05 mL of 100× L-arabinose stock solution was added to induce pKD46 lambda red expression. The culture was grown until it reached an OD600 of 0.4-0.6. The E. coli cells were then centrifuged at 2,000 rpm for 5 min. at 4° C., the supernatant was removed, and the cells were resuspended in 1 mL of 4° C. water. The E. coli cells were centrifuged at 2,000 rpm for 5 min. at 4° C., the supernatant was removed, and the cells were resuspended in 0.5 mL of 4° C. water. The E. coli were centrifuged at 2,000 rpm for 5 min. at 4° C., the supernatant was removed, and the cells were resuspended in 0.1 mL of 4° C. water. The electroporator was set to 2.5 kV, and 0.5 μg of the recombineering construct was added to the cells, mixed by pipetting, and pipetted into a sterile, chilled cuvette. The dry cuvette was placed into the sample chamber, and the electric pulse was applied. 1 mL of room-temperature SOC media was immediately added, and the mixture was transferred to a culture tube and incubated at 37° C. for 1 hr. The cells were spread out on an LB plate containing 35 μg/mL chloramphenicol and incubated overnight.

### L. Verification of Mutation

The presence of the mutation was verified by colony PCR. Colonies were picked with a pipette tip and resuspended in 20 μl of cold ddH2O by pipetting up and down. Three μl of the suspension was pipetted onto an index plate with appropriate antibiotic for later use. The index plate was grown at 37° C. overnight. A PCR master mix was made using 5 μl of 10×PCR buffer, 0.6 μl of 10 mM dNTPs, 0.4 μl of 50 mM Mg₂SO₄, 6.0 μl of 10× enhancer, and 3.0 μl of ddH2O (15 μl of master mix per PCR reaction). A 10 μM primer mix was made by mixing 2 μL of a primer unique to the CAT gene (100 μM stock) or genomic sequence neighboring the inserted CAT gene (100 μM stock) into 16 μL of ddH2O. Sequence of primers used is shown in Table 94. For each 20 μl reaction, 15 μL of the PCR master mix, 2.0 μL of the colony suspension (template), 2.0 μL of the primer mix, and 1.0 μL of Pfx Platinum DNA Pol were mixed in a PCR tube. The PCR thermocycler was programmed as follows, with steps 2-4 repeating 34 times: 1) 94° C. at 5:00 min., 2) 94° C. at 0:15 min., 3) 55° C. at 0:30 min., 4) 68° C. at 2:00 min., 5) 68° C. at 7:00 min., and then cooled to 4° C. The PCR products were analyzed by gel electrophoresis using 10 L of each amplicon and 2.5 μL 5× dye. The PCR product only forms if the CAT gene has inserted into the genome (thereby deleting and inactivating the Phage).

TABLE 94
Primers for Verifying Deletion of Phage3

| | | SEQ ID |
|---|---|---|
| Primer Name | Sequence | NO |
| Phage3 KO verify | TGGAGGCTTTAAGAAATACC | SEQ ID |
| FWD | | NO: 128 |
| Phage3 KO verify | GCTGGCGATTCAGGTTCATC | SEQ ID |
| REV | | NO: 129 |

J. Removal of Antibiotic Resistances from Mutant

The antibiotic resistance gene was removed with the plasmid pCP20. Plasmid pCP20 is a temperature-sensitive plasmid that expresses the Flippase recombinase that will recombine the FRT-sites thereby removing the CAT gene. The strain with deleted phage sequence was grown in LB media containing antibiotics at 37° C. until it reached an OD600 of 0.4-0.6. All tubes, solutions, and cuvettes were pre-chilled to 4° C. The cells were centrifuged at 2,000 rpm for 5 min. at 4° C., the supernatant was removed, and the cells were resuspended in 1 mL of 4° C. water. Cells were centrifuged at 2,000 rpm for 5 min. at 4° C., the supernatant was removed, and the cells were resuspended in 0.5 mL of 4° C. water. Cells were centrifuged at 2,000 rpm for 5 min. at 4° C., the supernatant was removed, and the cells were resuspended in 0.1 mL of 4° C. water. The electroporator was set to 2.5 kV, and 1 ng of pCP20 plasmid DNA was added to the cells, mixed by pipetting, and pipetted into a sterile, chilled cuvette. The dry cuvette was placed into the sample chamber, and the electric pulse was applied. One mL of room-temperature SOC media was immediately added, and the mixture was transferred to a culture tube and incubated at 30° C. for 1-3 hrs. Next, 200 μL of cells were spread on carbenicillin plates, 200 μL of cells were spread on chloramphenicol plates, and both were grown at 37° C. overnight. The carbenicillin plate contain cells with pCP20. The cells were incubated overnight, and colonies that did not grow to a sufficient OD600 overnight were further incubated for an additional 24 hrs. The chloramphenicol plate provides an indication of how many cells survived the electroporation. Transformants from the carbenicillin plate were purified non-selectively at 43° C. and allowed to grow overnight.

The purified transformants were tested for sensitivity to carbenicillin and chloramphenicol. A colony from the plate grown at 43° C. was picked and resuspended in 10 μL of LB media. Three μL of the cell suspension was pipetted onto each of three plates: 1) an LB plate with chloramphenicol incubated at 37° C., which tests for the presence or absence of the CAT gene in the genome of the host strain; 2) an LB plate with carbenicillin incubated at 30° C., which tests for the presence or absence of the beta-lactamase (CarbR) gene from the pCP20 plasmid; and 3) an LB plate without antibiotic incubated at 37° C. If no growth was observed on the chloramphenicol or carbenicillin plates for a colony, then both the CAT gene and the pCP20 plasmid were lost, and the colony was saved for further analysis. The saved colonies were restreaked onto an LB plate to obtain single colonies and grown overnight at 37° C. The deletion of the phage sequence was confirmed by sequencing the phage loci region of the genome and, more importantly, by phenotypically verifying the absence of plaque formation (essentially following the protocol as described in Examples 57 or 58, and as described for example in Example 60).

Table 95. lists the portion of the E. coli Nissle Phage 3 removed for inactivation of the Phage 3.

TABLE 95
Sequence removed from Phage 3

SEQ ID NO: 130
aatcgccggtgtactccgcgtcagaaaggtatacagccacggcagggagatcctgctcttcaagaaaaacagggcgcccgtcaaaccag
gtgaccgtgtcggtgatctcggctttcagtttggccagaatggctgcacgaattgcgctgtgtctgttcatcgcttcaggtggatcctcagttggt
ttttcagggctgcggaaagttctttgggcatatcgctttcaataaggcgctttgaaatagcggtgaaggccacggtgagcggtgtctcaagag
gaactttgaccacatcaatcggataacgggcctgacctacgcgccgcatgacctgccagcgcccgttcgcaagctgttggataaaagcgtta
cgaaaggtatagggcccgattttaaggacgctgcccgctccgtttctggccccttttttacgcgagagcctgacgcgcgccgtgccgagcttt
atcgcaggaagattaccgcggttgatttttatcgacgcgaccgggcgatcgtgacgggccttgcgcagacgggaacgctggcggaccaga
cgaaccggaagcccctttttccggttatcatcaactgttgcttctttcgctacagctttgctcccctggcttatcgttcttctggccaccctgttaagt
gcttttgcggttgcctcaggaacgattaaccggctgaggctgttcaggttctgaatagccctttccagtcctttcacagacatagcgcctcctca
ttcgagatggatgcggggttttccgttgaacatgtcatagcgggtaacgatcaggttcttaccgtcgtagtcgacgctgtcgtttcggcgtggct
ggtaaagctcagagaaaaccaccagcgaagtacctgttcccgacaatggccccatttcctcgagttgctcggcgggaacaacgtcatagct
gctgccattgatgatcgctgtctttcccatcttttttatagtggccgcgtccatgcgcgccgccatccggtcaaaggagttaggcattgatcttaa
cttcaacaacggtggtgtttgcccctgcatcttcccaggcgatgcccgcggcaacggcgtccgtttcttcgatcgtgattttgccgtccttcaga
tacacctgcgccccggcagtaaccgcatctgcggatacttttggcaggaggaaaacaccctcagtaaaaccgtccccggtatcgccagccg
ggatatcggtaattgccaccgcgataagttttccaacaacaaccgggtcgccgctgtgaacatcggttgcaccactgtttaccagagggatcg
ttttcccgtcctgcgcatagttcttagccataacttctccattcagcccctttcgaggctggtttcaggtataaaaaaagcccttacgggcgtctgt
ttgtcaggactgttttttactgaccagaggatttggtcatgccgcgatagtccagcggcgccacgccagcatcaatacgcactttcgtggcgat
accatcagtggtgaagccttcctgctgatcgatgtatggcgtgtcgacgccgttgagataagcgacctcaatggtgtcggtgcccttcgcggc
agccagataccaggctttcgcatcagcttcatccagacgtggttcggcaatgacttctgcaaagttctggatagggttaacgatcccggcattg
atgtctgcacctttaacactggccgacttgatggtctgatttgccagagtttccagggcgacgggcaccagcatgtaggccggacggatattc
agggttcgctccccctccttctgcagacgcatcagcttgcgcgattcgtccaggctggccacagaaattgcacccgagctcaggttcttgtga
tcggcatggaacagcgcctttccgtctgagagtttcgggtttttggtcagaatggcgtaaaccagatcgccaatcgttgctttcgccgcgcgcc
ccatcttcatcggtacgtcggtaagctggttcagatcgtcgttgatgatcgcctggcgagttactgagaagatttcaccatacgtggcaagcgc
gatggtttcgcctttgtcactggtagtgatgtacttgtactcagccccttcgcgaacctgtcgcagagaagggaacccacccataccgacacg
atgcgccgttttgaagtccgacagctggccttttttggtccactgctcgaaggtttcctgcgcctcgtcccagccctgaatcagcgctttgttcgc
aacatcaagcagaatgttgccaaagtcagaggtgctgtgggtcagcgccaggccaaccatctgcatcgggttgtagctggccacgccgata
cctttttctgtcagggccatacgcgcatactcgcgcagcgtcataccgttataaacgttatcccgctcctgaccttcgaacccggcacgcgcca
tcagtgcctggcgaataccatccgcgacgaagttaccgttgcccgcatgaatatgcggctgagtggttttattggacggcgtggccgttttacc
gagttctgccagcagcaaatctttcgccttatcgacggagcaatcagggtcggccacacactgattctgcagttccatgtgcttattaccgaac
atggcaaagagatcgccgatagcgttaacacgggttttctgctcagccaacacctgcgcgcggatcgcattttcatccggtgccgggtctgtt
tttgcctgcggtgcctgaggctgggtaataaccgggtcacgctgggtagtgttgcgcggcggggtgatcatgttgcgaatgctttttggcatttt
ttcaaattcctcaatacgttttgaatgaatacaggccatagcctgaagggatggtgtcacctggtcggcaaaacccagttcaaggcactcgctg
ccgttcatccaggtttcgtcctccagcattaccgcaatttcttcggtggattttccggttttctgtgcataagccgggataagaacggattcaacct
tgtcgagaagatccgcatagtcgcgcatatcgctcgcgtcaccaccagcaaacccccagggcttatggatcatcatcatcgtgttttcaggca
tgatgaccggattgcctaccatcgcaatcaccgaggccatggaggccgccagaccgtcgatatgtacggtaatcgccgcgccgtggtgctt
cagcgcgttataaatagcaattccgtcgaagacatcaccaccgggcgagttgatataaaggttgatgtgggtgacgtccccaagtgcccgga
gatcattgacgaactgtttcgccgttacgccccagtacccgatttcgtcataaataaaaatgtcggcctcactgttattgctggcctgcatgcgg
aaccacgaattactttttgcgctggctttcggacggtggcgcgcccggttctttggcttcggcactggtgcctcctttatcattggcggggtcgg
tgtcaaacaccaggccctgttcacggttctcgtcaacctcagctttacggcgtgacttaacatcatccgggttgcgaccgctggcacgtatcca
gtcggattcagtagcagcaccgccgcggatctgcgttttccaggcattcgcttctttaacgggatcaatccacggcataacgggccccgaata
aaccgcgttataaagcgagtccatatcaatgcctctcggcagcttgatttctccggcagcaatagccatcttgagccaggctcggtacatggg
ccgggtcactgaaccgatgaaccagtcctgaagaatcagatagccgtcggttgactcgacaagctcctgccgctgggcactgtacgttccgt
tgtagtttctggatgtgctggaaaagctgaggcgactgccggcggacacggcacgcagctgtccgttacgaaaagattcgaggttagggttc
gggcgatcggatttaatcatcccgatttcttccccggcctgcagttcgtcatagagcataccgggctgaatcatcagctcgcggtcatcgctgc
ttgaatcagaatcgaagctctgtccgtcgccttttttgatatacatgccgagtgccgcagcaattctggcagcagtaagctccgagtcctcgtat
tctttcagcgcgctcagacgcatcagaacaccagacaaaagagacgttccgcgggtctggtgcaggcgtcgggtgaatttgagatgcagca
tgttctctgcatctatctctttggtatcaaactgacgcccggatactggcaggcttttatagacctgatattttttcgggcgtccccagttatcgaca
aaaacgccctgattgagctgggtggcagcatcgctgttcatcggcacaaagtccggctccagcgcttccagccagaacggcacgccagca
accggctgaagaccatttccggtaccgcgaaccagctgagcaaatacctcaccgtcccggagccacgttcgcagcatcagccgctccagc
attgggcgggtaaactgggttgtgacatctggccttacggaccattcgccccactttcggcggatatcagtggccagctttttagcgatcttccc
gttactcagcatcggatgcggttcaactatgatgcccttcgcacccaccaccctttcttccagcttgtcgaaaacgccgatcaccagatcgtgg
ttgttatccagccagcgcgcctgctgcctcagcgaaaccgcccccatctggctgagctgatcggctgaacgattttccttctgggctttgtggg
tacgcgtttgctttaccgcctcatacgctttaataactgcgcgggcacgcaggcgtgaggctttccagcctggtgaaaacaggccaatcgcat
catctaaaaaactcatccaaacctcgccagcctgtagccgggtcgcccacggcgtttgttattgagcgttgccagtcgtcgctcccattcctga
cggccttttctgatttccgacaggttttcgagcgtcatctgctgcccgttgaaagtgattgatttcccctccagaacagacagctcggctgcagc
atagcggtcgatcatgttttgaatatctgctggattcacacccaacctcctgacgaagaccacggattagcctgctcggttacgggcttctcac
gttttggttttggtttagatttcggcgcaggcggcggggatggcatttcgccagcttccgtctgcgtgtcctcgatccacgtttcccgccgtgcc
cactcaggagctgacggccatttgattttttcgtaaccactaaggatggcgagcgcgtcggcataaacgagcaggtcaaatgcttcgtttgcg
ccccggccgggcttactccatttcccttcattcgagcgttcctcatacgtcagttcgtcatagaaccagctgcccagccaggcggggaaatgc
acatagccagggccgggtgaatcacgccacagcgcattattcacccggtctttaagggcatcggtctggagaagataaagaggcacatcac
cagtcgcctgtgcgcggcgcgttgatctgcccgtgttgtcgggaaacgttcgctggataagtttgctgcgcctgacgctgtcccctttgaaga
gatagatacgcttacccagcccctcacggcgacatctgcgccagaacttgtaggcattatccgtcacgccatcttcgccccctgagtccacg
gccatcgacatcagccgcatgccctttgatgggtcagctgcgagcggccacgttttatcaaagacgtcagtgagtaaaagatcccagtcctc
cggatagctcgccggatccacctgaatgctttcaccgttgccgtcgcagcgcagcgaatgccggatgttgtaacggtcaactatccagcgct
cacccatacttccataacccgtaatctgcacaacaaagcgccggttgcgcccggcctgcacgtccacggtcgcagtgagaaactgcacgc
cgttcggtaccgaacgttttgggacgtcttcggcacgctgctcgagcaattcacttttacgctgctccatgctggctcgcggcaaatagggcct
gccgaaatcggtgttgatcaccgtcttcagggtttcttcgctgcgcgtggattcatattcctgctcggcggtcagaaacttataaataagctgcg
cccaggtctggtaagcagctgccggaccttccatccagaaggaggcaatacgggaacgacggccatcaccgctaaccaggcctttcctgt
cgatggtttgcccgtcccggagccagacacatttcatgttaagcgcacgcttcatgtccggtgtgatcctgcctttacaggcagggcactgaa
gaaacgccgcttcgctggcaagcacaggatcgctgctgtcgcggtatccggtcatattgtccatttccggctggaaatattcgccgcaatgcg
ggcatggccagtaaagacgacggcggtcaccacggttatagagcgataaaattccggtggtcggaggggcttcatggggcgtggagcgc
cgccattttgtgtctctgatatccctcccgggcgagctctcaaccagcgtcatcccggaggacatgaatgtcgtggttcgtttcgatgccagtg
aaaaagcatccccctccccgtcgatatcttccggaaagcggtcataatccgtcagcgccacacttttatagtccgaggacgacatgatattga
cggatggccagcccagcttcagatagttaccggcgcggaatgtacggtcgtagacgttgttatcgttacgtcttgggcttagccgggttttaac
ttcagggctacagcgaaaagtacggtccaggcgttttttggaatgctcgcgcgctttttcctcagatacctgaattacaagcatatctgccggat
cgcagacaatgttataaacgatccagccgtcaatcagcccgatggttttacccgttcgcgctgggcccacaaacacaaccgcatcgtattcac
gcgatgccagacagttcatcggctcaatcacatagggtgccagatccggatcccacggaactgagtttcccgcccccattggcacgcgcat
ataagtactgaccgcatcggccaccggcatacgacgcggggctcgtaaaataccggaaacatcgcggcggatgtccctggcggatgccc
gctttgccatcagtcctcctcaggctgctcctcctcttttccagcgtcctgcaccttctccgccatctggtcgcgcagatcatcgataacgctttg
cacacgaactaccgcagcaggcgttaaagcacagtcgcgctcgagcacatccgggagggtttcaagtaccatgacgacggctttcgccat
caatgagaattctcgcgccacttcatctgcgggtattaactgccccgtatcctgttcgaacttcagcctctcgttctctgctttccagtgggacag
cctgtcagaagggggcatatcgtcgatgttggccgaaacggtagggatcatcagttcggtcagaatgtcggtcaccagatagagctttaactt
gctattgctgcctggagcaggttcaacatttttcagtctcgcggcaaccgtctgacggtgtacgccggttatccctgccagctggttgatattga
gttttaaagtggcaatttcctggtccatgatggtgaacactttttgaacgattcgacatgttgcgaaaatggcctctaattaaatcaaagacctgc
gcacatgatgatgatgaccctggatccgaaaaactagccgtttcccgcgagcacgccgccccgtggcagggtccccctccgggagtacctt
ttgataataattatcaattgcacactatcgacggcactgctgccagataacaccaccggggaaacattccatcatgatggccgtgcggacata
ggaagccagttcatccatcgctttcttgtctgctgccatttgctttgtgacatccagcgccgcacattcagcagcgtttttcagcgcgttttcgatc
aacgtttcaatgttggtatcaacaccaggtttaactttgaacttatcggcactgacggttaccttgttctgcgctggctcatcacgctggatacca
aggctgatgttgtagatattggtcaccggctgaggtgtttcgattgccgctgcgtggatagcaccatttgcgatagcggcgtccttgatgaatg
acactccattgcgaataagttcgaaggagacggtgtcacgaatgcgctggtccagctcgtcgattgccttttgtgcagcagaggtatcaatctc
aacgccaagcgtcatcgaagcgcaatattgctgctcaccaaaacgcgtattgaccaggtgttcaacggcaaatttctgcccttctgatgtcag
aaaggtaaagtgattttctttctggtattcagttgctgtgtgtctggtttcagcaaaaccaagctcgcgcaattcggctgtgccagatttagaagg
cagatcaccagacagcaacgcgccacggaaaaacagcgcataaagcacttcattagcagcgccagatagcgtaatgattttgttactcatgg
aatatttccttttaggcgtgagcctgtcgcacggcaatgccgcccgagaggtaaacgcaacctaacggcatcacccaggctcactactgaaa
gactctctttgatgtgcgcgtgcgatgcgcgtagaagactgatttatcaacctgtctttatatcaggattcattacctgactatttgtgggtaaagtt
cgtagtgcgctgatcgtgcaaaatgattttagttgggaacagttcgcaactctgtcccataaaaatcagcatattcccatctatcccatatccagc
gcattgaccatcgggatactgaagggagattccatcatctcttagaaagatcaccatctcttttgtttcaatttgcatatagctacctggaggattt
atgaatgcaaggattttcatggactattaccatgagattgattttccatctttattcgcgagagcagtggaaagcgatgacgatgtgggtactaca
ttgcgcattcacctactttgtgagcgcatggtcgaagcatggatatgcgcatgctgtgactgccaagatctctttggaagagataaaaacaaac
ttttaatcgaatgtaatactaaaatatccatggcgggaaacctgggaatccccccggaacttatgaaatcacttaaaaccatcaactcaatgcgt
aatgaccttgcacacaatccatcaatacaaagcattgctgattcaaggatccagagcctgaaggatactctgactgaatactttaaacagcatc
caacggaacccagcatggaagaatcaaaactgggtatttttaacgccgagaatcaattaaccgaagaagtttccttagatagtgacagttcaa
aaaacagacttaagttaatcttgctgttcagcaagttaatgcaggcgttaatgcaattagttgcagctaatcataatgggcgctgggataaccaa
tttagccaattcgtttaccatgtgaccatgaacgcaacaaagagataaatccaagcccgttttgtacgggctgttgcattatcacaggcactcag
tgaatgcctgctgtaatgccgctagtcgtcgagttgcaacacaccgtg

Table E. lists the Phage 3 genes that were inactivated by the deletion.

TABLE E
Phage 3 Genes inactivated by the deletion

| | | | |
|---|---|---|---|
| ECOLIN_10110 | 1 . . . 160 | Minor tail | GI: |
| | | protein U | 660512026 |
| ECOLIN_10115 | 157 . . . 729 | tail protein | GI: |
| | | | 660512027 |
| ECOLIN_10120 | 745 . . . 987 | DNA | GI: |
| | | breaking- | 660512028 |
| | | rejoining | |
| | | protein | |
| ECOLIN_10125 | 1013 . . . 1339 | hypothetical | GI: |
| | | protein | 660512029 |
| ECOLIN_10130 | 1422 . . . 3368 | peptidase | GI: |
| | | S14 | 660512030 |
| ECOLIN_10135 | 3382 . . . 4881 | capsid | GI: |
| | | protein | 660512031 |
| ECOLIN_10140 | 4878 . . . 5093 | hypothetical | GI: |
| | | protein | 660512032 |
| ECOLIN_10145 | 5090 . . . 7192 | DNA | GI: |
| | | packaging | 660512033 |
| | | protein | |
| ECOLIN_10150 | 7192 . . . 7680 | terminase | GI: |
| | | | 660512034 |
| ECOLIN_10160 | 7864 . . . 8592 | hypothetical | GI: |
| | | protein | 660512035 |
| ECOLIN_10165 | 8767 . . . 8997 | hypothetical | GI: |
| | | protein | 660512036 |
| ECOLIN_10170 | 8996 . . . 9592 | hypothetical | GI: |
| | | protein | 660512037 |
| ECOLIN_10175 | 9661 . . . 9687 | hypothetical | GI: |
| | | protein | 660512038 |

Table F. shows the sequence of Phage 3 comprising the deletion.

TABLE F
Phage 3 sequence with Deletion

SEQ ID NO: 281
aggcctctcctcgcgagaggcattttttatttgatgggataaagatctttgcgcttatacggttggatttcgcccggtttgcgagttttcagcaatttta
atatccaggtgtattgttctggtcgcggaccaacaaaaatctcgacttcttcattcatccgccgcgcaatcgtatgatcatccgcctctaacagatca
tccatcggtgggcgcacctgaatcgtcagacgatgcgtcttgccatcataaatcggaaatagcggtacaacgcgcgcacggcacactttcatca
aacgaccaatcgcgggcaacgtcgctttataggtggcaaagaaatcaacaaattcgctgtgttctgggccatgatcctgatcgggtaaataatat
ccccagtaaccctgacgtaccgactggatgaatggtttaataccatcatttctcgcatgcagacgaccaccaaagcgacggcgcaccgtgttcc
agacataatcaaaaaccgggttgccctgattatggaacatcgctgccattttctgcccttgcgaggccatcagcatggcaggaatatcgacggcc
caaccgtgcggcaccagaaaaatcactttctcgttattacgtcgtatctcttcgatgatctccagcccttgccagtcaacgcgcggctgaattttctc
cggcccgcgtattgccaactcagccatcattaccatcgcttgcggcgcggtggcaaacatctcatctacaatcgcttcgcgttcagcttcactacg
ttctggaaagcagagcgacagattgattaacgcacgacggcgtgagctttttcccagtcgtccggcaaaacgtcccagccgtgccagaatggg
atcacggaactttggcggcgttaaagcgatacccgccatcgctgctacgcccagccatgctccccagtagcgcgggtggcgaaaggatttatc
aaactcaggaatgtattcgctattattttttttcgtttccatgcttttccagtttcggataaggcaaaaatcaatctggtgatagtgtagcggcgcaactt
gccccgcaccaaataaaaaagccggtactgactgcgtaccggctgcgaatggatgttaattaatcaaaccgtagctgcggcacaatctctttgg
cctgtgccaggaattcgcgacgatcggagccggtcagcccttcggtacgcggcagttttgccgtcagcgggtttacggcctgctggtttatccat
acttcatagtgcagatgcggcccggttgaacgtccggtattaccggaaagcgcgatacggtcgccacgtttcaccttctgtcccggtttcaccag
gatcttgcgcaagtgcatataacgcgtggtgtagctgcgaccatgacgaatagccacataataacctgctgcgccactacgtttggcaaccacc
acttcaccgtcacccactgaaagcactggcgtaccttgtggcatggcaaaatcaacacctctgtgtggcgcaacgcgaccggtcaccggatta
gtacgacgcgggttaaagttagatgagatacggaactgtttcgccgtcgggaatcgcaagaatcctttcgccagaccagtaccgttacgatcgta
gaatttgccatcttcagcgcggattgcgtaataatctttaccttctgaacgcaaacgtacgcccagcagctggctttgctcacgtttaccatcaagc
atttctcgtgacattaacaccgcaaattcatcgccttttttcagtttgcggaaatccatttgccactgcatggctttaatcactgcgctcacttcggcgc
tggttaaaccggcgtttctggcgctggcaacaaagcttcccccgacggtacctttcagcagattgttgacccactctccttgctgcatttcgctggt
cattttaaaaccgttagcggcagtacggtcataggttcgggtttcacgacgagacacttcccaggtgaggcgctgcagttcgccgtccgcggtta
atgtccaggagagttgttgaccgattttcaggttacgcaattctttgtcggcagcagccagttgggtgatatcacccatatcaataccatactgattg
agaatgctgcttagcgtatcgccagtggaaacaacatattcatgcacgcccgcttcaccggcgattttgtcatccagttcgtcctggggaatggct
tcatcttcttgtgcagcttgatcaatcggctcactggcttcaggtaagagcgaacgaatttcgttctgttccagctcaatggttttgacaattggcgtg
gcatcgcggtgataaacatagggccgccagacagcgacggccagagtaagaacggtgagcgaccccaacataacgcggtgtggtcgcggt
aaattattaaacgccagggcgacagagcgggctatctgttgcacgtaatcacttcctcattaatctcctttcaggcagctcgcatactggttggcta
attgattcaggaattctgaatagcttgttttacccagtttgatattcgtccccaggggatccaacgttcccatacgaacggatgtccctcgtgcgacg
ctctcaacgaccgctggcctgaactgtggctcagcaaaaacgcaggttgctttttgctcaaccaactgtgttcttatttcatgtaaacgctgcgcgc
caggttgaatctcagggttaacggtaaaatgaccaagcggtgtcagtccgaactgtttttcgaaatagccgtaagcatcgtgaaaaacgaaataa
cctttccccttgagcggcgcgagctcgttaccaacctgcttttcggttgaggctaattgtgcctcaaaatccttcaggttggcgtcaagtttggctcg
actttgcggcataagttccactaattttccatggattgcaaccgctgtagcccgcgctatctctggggaaagccaaagatgcatgttgaaatcgcc
gtgatggtgatcttcgtcacttttttccgcgtggtcgtgatcatcatcatcgccgtgaatacttttcatcagcagcggtttcacattctctagctgcgca
atcgttacctgtttcgcttcaggtaatttacttaccggtttttgcatgaacgcttccatctccgggccaacccaaacgactaagtccgcgttctgtaag
cgttttacatctgatggacgcagtgaataatcatgttctgaagccccgtcaggtagtaaaacctccgtttctgttaccccatcagcaatggcagaag
cgatgaacccaacgggtttaagcgaagcgacaacggcagcatctgcggcctgtgttgcaccgccccagagagcggcggataatgctgcgaa
aagaagcgtttttttatgtaacataatgcgaccaatcatcgtaatgaatatgagaagtgtgatattataacatttcatgactactgcaagactaaaatta
acatgacaagtctggtttccctggaaaatgtctcggtttcttttggccaacgccgcgtcctctctgatgtgtcgctggaacttaaacctggaaaaatt
ttgactttacttgggccaaacggcgcaggtaagtcgacactggtacgggtagtgctcgggctggtaacacccgatgaaggggttatcaagcgc
aacggaaaactgcgcatcggctatgtaccgcagaagctgtatctcgacaccacgttgccactgaccgtaaaccgttttttacgcttacgccctgg
cacacataaagaagatattttgcctgcactgaaacgtgtccaggccgggcatctgattaacgcaccgatgcaaaagctctcgggtggcgaaac
gcagcgtgtactgttagcgcgagcattgttaaatcgaccgcaattattagtgctggatgaacccactcagggcgtggatgtgaatggtcaggtgg
cgttatatgaccttattgaccaactgcgtcgcgaactggattgtggcgttttaatggtatctcacgatctgcatctggtaatggcaaaaaccgatgaa
gtgctttgcctgaatcaccacatttgttgttccggcacaccggaagttgtttccctgcatccggagtttatttctatgtttggtcctcgtggtgctgaac
aactgggtatctatcgccatcatcataatcatcgtcacgatttacagggacgaattgttttgcgtcggggaaatgatcgctcatgattgaattattattt
cccggttggttagccgggatcatgctcgcctgtgccgcgggtccgctgggttcgtttgtagtctggcgtcgtatgtcttatttcggtgatacgctgg
ctcatgcctcattacttggcgtcgcgtttggtttgttgctggacgtgaatccattctatgcggtgattgccgttacgctgctgctggcgggcggtctg
gtatggctggagaagcgtccacagctggcgatcgacacgttattagggattatggcgcacagtgccctgtcgctgggcctggtggtcgttagtc
tgatgtctaatattcgtgttgatttgatggcttacctgttcggtgatttactggcagtgacgccagaagatctcatctctattgcgattggcgtggtcat
cgtggtggctattttgttctggcaatggcgcaatttgctgtcgatgacgattagcccggatctggcgtttgttgatggtgtgaaattacagcgcgtga
aattgttgttgatgctggtgacggcattgacgattggtgtagcgatgaaattcgtcggcgcgttgattattacttcactgctgattattcctgctgctac
tgcacgtcgctttgcccgcacgccggaacagatggctggtgtcgctgttttggtggggatggtggcagtgactggcggtttaaccttttccgcatt
ttacgatacacctgcaggcccgtcggtggtgctatgcgcggcactgttatttattatcagtatgatgaaaaagcaggccagctaatctgtcgctga
acacatttgtcggatgcggcgcgagcgccttatcccacctgcggttcgctatctctggtaggcctgataagacgcgaacagcgtcgcatcaggc
acactgccagtgtcggatgcggctcgagcgaccaatccgacttacggcatttctggcggcgtgatgccgaagtggttccacgcccgcactgtc
gccatacgcccgcgcggtgtacgctgcaaaaagccttgctgaatcaaataaggttccagtacatcctcaatggtttcacgttcttcgccaatggct
gccgccaggttatccagacctaccggcccaccaaagaacttatcgattaccgccagcaacaatttgcggtccatataatcgaaaccttcagcatc
gacattcaacatatccagcgcctgagcagcgatatctgccgagatggtgccatcgtgcttcacttcagcgaaatcacgcactcgacgcagcaga
cggttggcaatacgtggcgtaccgcgcgcacgacgagcaacttccagcgcgccgtcatcactcatctcaagccccataaagcgtgcgctgcg
actgacgatatattgcagatccggcacctgataaaactccagacgttgcacaataccaaaacgatcgcgcaacggtgatgtcagcgaacctgc
gcgcgtggttgcaccaatcagggtaaacggcggcaaatcaattttaatggagcgtgccgccggaccttcaccaatcatgatatccagttggtaat
cttccattgccggatacaacacctcttccaccactggtgaaagacggtggatctcatcaataaacagtacatcgtgtggttcaaggttagtgagca
ttgctgccagatcgcccgccttttccagcaccggaccagaagtcgtgcgtaaattaacgcccatttcattggcgacaatattggcaagcgtagtttt
acccaaccccggaggaccaaaaatcaatagatgatcgagggcatcgccgcgcagtttcgctgctttgatgaaaatctccatctgcgaacgaac
ctgcggctgaccaacatactcttccagtaatttagggcgaatggcgcgatctgccacatcttccggcaaagtggtaccggcagaaatcagacgg
tctgcttcaatcatcctttacctcataacgcggcgcgtagggcttcgcgaattaatgtttcactgctggcgtcagggcgagcgattttgctcaccat
gcggcttgcttcttgtggtttatagcccagtgccaccagcgcagcaaccgcttcctgttcagcatcgtcggtcgccgggctggcaggagacgtg
agtaccaggtcggcggctggcgtaaagagatcgccatgcaaacctttaaatcggtctttcatttcgacaatcaagcgttcggcggtttttttgccaa
tacccggcagtttcaccagtgcccccacttcttcacgctcaacggcattaacgaactgctgcgctgacattccggagaggatcgccagcgccaa
cttcgggccgacgccgttggttttgatcaactctttgaacaacgtgcgctcttgtttattgttaaaaccgtacagcagttgcgcgtcttcacgcacca
caaagtgggtgaaaacgatcgcttcctgacccgcttcagggagttcataaaaacaggtcatcggcatatgcacttcatagcctacgccgcccac
ttcaattaacaccagcgggggttgtttttcaatgatgatgcctctgagtctgcctatcacatgacgctcctgcgtaatgaatcaaagataatgctgtat
gataaaaaaatgctggatagatatccagcgaaggatgaagaaaacttgcgaggtgtctcgatgatctgaaaaatggcgcagtataatttattctac
agattatattggaagcaaatatttaaatattacatattcagcgaagaaatgtgtaataaaaatacacattgcgacccctgaaaaaaataaattttttatg
ctattacgtatattcatatctatttcaatggaatgacaacgtgaatattaattatcctgctgaatatgaaattggtgatatcgtctttacatgtataagtgct
gccttatttggtcaaatatcagctgcatcaaattgctggagtaatcacgtcgggatcattatcggtcataacggtgaagactttctggttgcagaaa
gccgtgttcccctctcaaccatcactacgctatcccgttttattaaacgctctgctaatcaacgctatgctataaagcgattagacgccggactaac
agaacaacaaaatcaacgaattgttgaacaggttccttcccggctacgcaaaatttaccacaccggttttaaatacgaatcttcgcgccagttctgt
tcaaaatttgtttttgatatttataaagaggcgctatgtattccggtgggtgaaatagagacgtttggagaattgttaaatagcaatccaaatgcaaaa
ctcactttctggaaattctggttcttaggttctattccgtgggagcgtaaaaccgtcacgccagccagtttgtggcatcatccgggtttggtgttgatt
cacgcggtgggagttgaaacgcctcagcctgaactgaccgaggcggtataacttaacgcagtcgccctctcgccaggttcagtcgcgattcgc
tcatttgcatcgcattctgactaacgtggcagtgggtgatggcaatcgccagcgcatcggcggcatccgcctgtggattagcgggcagtttcag
caaggtgcggaccatatgctgcacctggcttttttcggcactaccaatacctaccactgtttgctttacctgacgtgccgcatattcaaataccggc
aattcctgattcaccgccgccacaatcgccacgccgcgcgcctgccccagtttcagggctgagtcagcgttcttcgccataaagacctgttcaat
ggcgaaataatcaggctggaattgggtgatgatttccgtcacgcccgcatagatgagcttcagacgagacggtaaatcatccactttggtgcgta
tgcatccgctacccaggtaggacagttgcctgcctacctggcggatgacgccatagccggtcacgcgcgaacccgggtcaatgccgagaata
atagccatcacgcgtctccgttttgctgtttagcaggcctcatcagagagtcgctgcaacctcatcagagatttcaccgttatggtaaacttcctgca
cgtcgtcgcaatcttccagcatatcgatcagacgcatcagtttcggtgcggtttctgcatccatatcagctttggtggacgggatcatggaaacttc
cgcgctgtctgctttcagacctgccgcttccagagcgtcgcgtactttgcccatttcttcccatgcagtgtagacatcaatcgcgccgtcatcatag
gtcacaacgtcttcagcaccggcttccagggctgcttccatgatggtgtcttcatcgcctttctcgaaggagatcacgccttttttgctgaacaaata
agctacggaaccatcagtaccgaggttaccgccacatttgctgaatgcatgacgcacttcagcaacggtacggttgcggttgtcagacagacat
tcaatcatgattgccgtgccgccaggaccgtaaccttcgtagatgatggtttccatgtttgcatcatcatcaccgcccacacctcgtgcaattgcgc
ggttcagagtgtcacgggtcatgttgttagacagtgctttatcaattgctgcacgcaaacgcgggttagcgtccggatcaccaccgcccagctta
gccgcggttaccagctcacgaatgattttagtgaagattttaccgcgcttagcatcctgcgcagctttacgatgtctggtgttggcccatttactatg
acctgccataaaaatatctccagatagccctgcctgttcaggcagcgttaattacaaactgttcaatcgcctgccggttgctccaggacttagtga
gcgccgccgcagcagacgcatcaagccacttgtaagccagatgttcagtgaaaacgatctggcgctcgtgcggaagcgcaagacagaacca
tgattccgtattacgcgtcacgcccggcgcatagcgatgacgtaaatgtgaaaaaatttcaaactctaccgtgcgctgacagtcaattaaggtca
gttgttcagcgacaacatcaatggtgacctcttcctttacttcgcgcatggcagcttgcggcgcggtttcaccctcttccacgctgccggttaccga
ctgccagaaatcgggatcgtcacgccgctgcaacatcagcacccgtttcgtatcttgtgcgtagatgaccactaagatcgaaacgggacgcttat
aagccatatcagttattctcagccttcttcacaacctgaatgctcagctcagccagtgcagtcgggttagcaaagctcggcgcttcagtcatcaaa
cacgctgccgccgtggttttcgggaaggcgataacgtcacggatattgtcggtgccggtcagcagcatcgtcagacggtcaagaccgaatgcc
aaacctgcgtgcggcggagtaccgtatttcagggcgtcgagcaggaagccgaatttctcgcgctgttcctcttcgttgatacccagaataccaaa
caccgtctgctgcatatcaccattatggatacgcacagaaccaccgcccacttcgtaaccattgatgaccatatcgtaagcgttagccaccgcatt
ttccggtgcagctttcagttctgctgccgtcatgtctttcggtgaggtgaacggatggtgcattgctgtcaggccgccttcaccgtcgtcttcaaac
atcgggaagtcgataacccacagcggtgcccatttgctttcgtcggtcagaccaaggtctttacccactttcaggcgcagtgcgcccatcgcgtc
ggcaacaattttcttgttgtcggcaccgaagaaaatcatatcgccatcttgcgcgccagtacgctccaggatggcttcgatgatttctgcattaagg
aacttcgctaccgggctattgataccttccagacctttcgcgcgttcgttaactttgatgtaagccagacctttcgcgccgtagattttaacgaagtta
ccgtattcgtcgatctgcttacgggtcaacgatgcgccgcccggaacacgcagagcggcaacacggcctttcggatcgttcgccggacctgca
aatactgcaaactcaacagatttcagcagatcggcaacgtcggtcagttccatcgggttacgcagatccggtttatcagaaccataacggcgttct
gcttctgcaaaggtcattaccgggaaatcgcccagatccacgcccttcacttccagccacagatgacgcaccagcgcttccatcacttcacgca
cttgcggcgcggtcatgaaagaagtttccacatcgatctgagtaaattcaggctgacggtcagcacgcaggtcttcgtcacggaagcatttaacg
atctgatagtagcggtcaaagccggacatcatcagtagctgtttgaacaactgcggggattgcggcagcgcgtagaatttacctttgtgcacacg
agaaggcaccaggtagtcacgcgcgccttcaggcgtggctttggtcagcatcggagtttcgatgtcgaggaagccgtggtcatccataaaacg
gcgcaccaggctggtgattttagcgcgggttttcaggcgctgagccatttccgggcgacgcaggtcgaggtagcggtatttcagacgcgcttct
tcggtgttgacgtggttagagtcaagcggcagaacatctgcacggttgatgatagtcagcgaggacgccagtacttcgatttcgccagtcgccat
atcgcggttaatatttttttcgtcacgcgcacgtacggtgcccgtgacctgaatgcagaactcattacgcagttcagaggccagctttaacgcgtc
cgcacgatccggatcgaaaaatacctgcacgataccttcgcggtcgcgcatatcgatgaagatcaggctaccaagatcacgacgacggttgac
ccaaccacacagagtcacctgctgccccacgtgggacaaacggagctgtccacaatattctgtacgcatgagatatcccttaacttagctgccg
gcggatgccccctgctgcgcaggtgaccaagtcgcagcgttagctgtatgtcacaactgaatgaaaaaaggcggctattatactggaaattctg
ccgcaccgtaagagcctggcccgcgctggaacgcctcgttaccactttatatcgggcctgaaatcagactctacgccagtttgctataaaggtgt
tgcccgaactcataaaaattaacaaaatttgtcgttccgccatcggctaatcgcattaaggtgagaggcacgattttgttttgtcaggagtcatcatg
cttgaacttaatgctaaaaccaccgcgctggtggtgattgatttacaagaaggcatcttgccttttgccggaggtccacatactgccgatgaggtg
gttaatcgcgccgggaagctggcggcgaaatttcgcgccagcggtcagcccgtgtttctggtgcgcgttggctggtctgccgattacgccgaa
gcattaaaacagccggttgatgccccctcccccgcaaaagtgttgcccgaaaactggtggcaacatcctgctgcattaggtgcaaccgacagc
gatatcgaaatcatcaaacgtcaatggggtgcgttttacggtacggatctggagttgcaattacgccgccggggtatcgatacaatagtgttatgt
gggatctcgaccaatatcggtgttgaatccaccgcccgcaatgcctgggaactcggttttaatctggtgattgccgaagatgcctgtagcgccgc
tagcgccgagcagcacaataacagcattaatcatatctacccgcgcatcgcccgtgtgcgtagcgttgaagagatcctcaacgcgttatgattta
catcggtttgccacaatggtcgcatcctaaatgggtgcggttggggatcaccagccttgaagagtatgcccgccactttaactgcgtgacgcgg
gcattttaaaaatcactaaagaacgcccaagagcatgtgttttctttagtttattcaatgcattaaaaaatagtttcgcatgaaattcggtaaacttcat
gtgtgcaataatgtcccattcatgccccaaaatgccccaaagcagacatttttgccccaagtatgccccacaagtcacgtcttcaagtcgtctatat
ccatagcacaccgagttacattcttgcatccggggtgtcgacaatacctactttattgagtgtgcgagaattaccaggaacctttccacaatgtagt
agtctaatagtcgaatccatctaacattaagaagcgttatgatcactagcctctcattgatatcttctgtaatagtcactctatgtatcatggtgttcgct
acagtaaaggtagggattggtttgtctaacaatccagacagaaatgataattaacctcaaccacgtaaccacacttcatacttcatacttcacttaac
agtgaagtgctcacatcaccgggcagtcatcaaactccgcattcctggcatcattaatgatgtacgtgatcactccaaatatagcgggtgcagaa
ctgtaaccatcatcatctgctggcagcgcttcccttctcccgttatccagattaaccaggtgcggctgaggatgagtccgatatcgcttgatcctga
attccccgtcgattgcacatatcagcagtgaaccatcgcaggcagtaagtgacgcatccacaacaagcaacgctccctggattatcccttccctg
aaatgtgaacgcgatgcccgcatgaaataagtcgctgcgggctgactgattagctgctgatcgagggagattcgtgtttcaacataatctgccgc
aggtgaaggaaatcccatgtttacgccctctcttgaataccggataaaaacacagtataaatactgtatatccatccagcaaagaggcaatgagc
aatgttcgtggaactcgtttatgacaaaaggaattttgatggtctgcccggtgcaaaagatatcattctgggcgagttaactaagagagttcaccg
gatcttccccgatgctgatgttcgggttaaaccgatgatgacactgccggcgatcaacactgacgccagcaagcatgagaaggaacagataag
ccgtactgttcaggaaatgtttgaagaggctgaattctggttagtgagtgagtaaagattttcaatgcccgccacagttacgtattgattatgctgtg
gaggatattcattttcgtaaacgttggtttgggagaagcggcaaaacggaatgtgggaacaggggaaaatcagataccagatatgtctgcatttc
catctggcaataactggtttcagttaccaagtggacatatcgttcagatattttccatgaacgttcttggtgcagatgctaatggcacgtcagctaatt
accccattgcttttccaacaacgatgattgctgtcagtgctctatggtctgatgggactgtagcaaatgcaccgacatacaagatgatggggaaca
cgactaacagaacaactttgacgataaaagtatcagccagctcaggtacttacgggacaatgattattgcggtgggacgataatatgaataaata
cagttactctccttcagaaaatgccttttatgctgttgcgttaaaaaatacctatgaattgagtggcacatggccagctgatgcattagatattcctgat
gacatttctgtaaaatatatggcggaaccgccacaagggaaaatccgagttgcaggggaaaatggttttcccacatgggctgaaatacctccac
catcacatgaggaacttattgaacaggccgaatcagagaggcaattattgattaaccaggccaacgaatacatgaacagtaaacaatggcccg
gtaaagccgctattggtcgtctgaaaggcgaggaactggcacaatataattcgtggctggattatctggacgcactggaactggtcgatacttcc
ggtacgcccgatattgaatggcctacgcctccggcagttcaggccagatgacatccggcgcggtgctggtatctgttgcagtcaccgcgtcaat
gtaatccagcacggcgttaagtcgggttgtttctgcctgagtcagtttccgtccggcctgtaatttcagctgaatcagactaatggaagccattgct
gcatcaatcagtgattggcgctgtgcttctgccgcttctactgaggcaccgtgttgtgcctcagtatctgtcacccatttctcaccatcccatttatcat
atggcgttaacggtgaaagcgtgacataaccgtttttgatggcaccgatataatccactgtaacagctgcgccattttcgattgagtaaacagtctc
attgcgatggtcttcctcatggctccatcccttacctgtaaatactgccactcttcccggaatgttttcgtccgggtcaataccagtggaacaggcg
ggcatacttacgccagtattaatatattcatcagaccagcccgtatattcagacgttactgcatcataataaaaacaacgcatatcacccggcactg
cagccagcccattttcatcaaaaacaggtttcattatttagccctcaccagaaagttaaatgcaatatttcgcggtctgacagcaacaaaattcaca
ccatcacccacagagttactgttgaaattaaatcgtgaaaatcctggctgatttccggcgatgccatcatgaaagttaattgcgtgtccagcacctc
cgcctatattcccggcaaactgagaaaagtttgtagcttcctgccagcttaataattcgcgaccaccatctgcacctcgcccgtcatcccagacac
gaatgaaatcaccgcgggcttcaggtaataccagcgaaggaaacactttcgccagcacaggataatcagtggcagagaatttcgcgccgttga
acttcaaaaacaccatactggaccagctgtcgattacagtatttggcattgcagcggacggccagaagaacggaacgccaatagctggagaac
cttctcccaaaccaaggtttgtgcgagcgtctgcggcattcgttgcgccggttccgccgtctgcgacagtaaccgcaccgttgctccctttctgcg
caagtttaccgatgcctgggatggttacggcggtgccgttgatggtaactgtgatgctttggtttgctgaggtggtggcgaacgtctcccacgcg
ccaatattctcgtcgtactctttgatgagctgtgacatggcctgcgccaggccgtcgactgagatattgtccgacacaaggattccatacttctggc
cgctcagcgccggggaaactgctggcgtaaccgtcattgacgtggcgctgttcacggatgaaatctgaaacagctgcaccgggttagacatca
cgataatcgtctggccagcgcggacctggctggcgggagctgtccagtttgtgccggagccggttgcggtatttccgttaatagagatagttcc
ggtgctataaatcataacaactcctaaatttagacaacatgaagcccggagaggtatataaccctcaccagaaataatttctgaattggtttttaata
catgttgggcaacgccagtgttggcatagctatagttgtatcaaatgccattgaccacccacccaaataataattgccgactactttattcctttctgc
tctgacattcccacctgtcattacaattcctttatacctaatatttccgtaaccaccaacgtgtcgacagttagccccggtataaacaatctggcaaaa
ttcatcaccaatattcaggttcgcattggcgatatttattgtcccatcaaaaatgaatggcttcgtgcaagtaagaagacgtttgcgttgacgattggt
ttattcaatccatcaactaaaatatcatgtaaacgaattgccataacatttcccactttattttacttactcaacacaacaagtatattaaaattgaacctt
gtcaacaacacaaaggagtcccaatgaaactcgctctaattatgctgccattatgtctgtccctcactgcatgtggtaatggtttaaataccggtaa
accaaattccggtgtcattccaaaacctttggatcgagatggtaacggttctttaatttatgataccgaaaaccttccaatgacggggcagtggtgt
cacgagattgatcacgaataccgacgaatcggtagcccttctaactgtgttatagactactaaatattaacccctcaaaagaggggttaatattttaa
cctgtgaatgaaccagatccgtgtgatactatgatagtaggcgaagaaattgacgcacccctgttattttgagcggacactttaatacgcactgtta
cgtttggacctgttacaaccgcagaatgcatgataagtctctccatgtcttgcacggatgaaccactctcattgccgttaatgtcgattgttcctgca
ccattacctttaacgtttgctataacacagacgtgtcttgcgtgcccagaactggatgaatcattataagtcattaccttttctaaatatccgtcactag
accgactcacattcgtccctgtgtgcatatttgcaatgtccccgataaaactttgggcttccacagtccctttgaatttaccgcttgttgcttggatctc
accagtaaagctaccgccactagcatatactacacctctgacggtcacattgttgaattcagcatctccagctttattcaacttccaaccagcagaa
ccagctgcatagttgttggactggatatagttaccgatttttgcgttctcaatggtgccgtcctggatgaagctggcccggatgaatgtctgcccgtt
ctggatcacgaacggcaaagctacgctatttccggctgccgtggtgacggcgaagcggtcagccaggaagataacctgcgactgcatgccg
gatggcgtattctccacgccgatacccatccccgcggcgtaatactgcccgttgctggagacaccaaccttgatgttgtacatcgcgctgagttc
gccattaacgttggctatagcctgagcgttagtggtgatggcggaggtatgcccgttcacggtcgccgtgatgccgtttatctgcgtggcggtgg
cctgctgatagtcggagagcgtctgattcaggctgttgatggatgccttgttgccgttgacgtccgtctgcaggctcagcaatgaacgtgctgtgg
cttccttctcactgacgatcacctcgtcgagacggtccagattcgcgctgttgccggcgaccgatgcagaaagggttttacgcgtggccacctga
gcgaggttggcctggattatcgcaattgcagagttcttcaccccgcccgtcatgccgtccatagaaacgctgatgttgtcgattcgctggcccag
ggcggtatcagccgtcgcaacggtctgctcaagctgactgagtgaagacgaaacattcccgaccgtgctggaaagctcattaacgctggtctg
aaccttcccgacgtcctgggcatttttggcgatatctttcgcttgctgctccagttcgtcgttggcctgtttgatatcgttagccatgccagcaatttttt
cgttgctgtccaccgcgttctcgatcaggtctttgaacgtttccgactctttcatatcctccagaatgtcattagttatttcgctgacatctatcgagga
cgtgcccatgatccagtcggtccagtccccggcgttaccgatacggtcaatcaggcgcgcgcggtaccactggcgaacgccggcaggcatg
gggccatgctgataatctgcagccgggtacggcaccaggaccagcagttcaggattggcgtagtcggcagttgtggcgcgctgaatctctgta
taggccgtgtcgcctgagccatccggaaatttccaggtcaggtcgatatgccagaccacatcttcggtcgccaggaagttgagcggagtaccc
ggttttcccgttttaccggagagataagttgtttcaccgtatccccatggtgacgacgtatcctgcgcattcagcgcccgtacgcgcacgtcatag
ctgcccgaataaatgccctgaaccgagaaaccctgcgcgctggtaaccggaacgtttatccagtccccgttgtccttacgccactgggcaacat
accggattgcgccctctaccttatcccatgacacgtccaggcttgctacagtcagcccctgagacacatgatcgctctcagtcaccacgatattct
tcggagcagacaggacgcttatcggcgtgacggtgatcgggggagactcgacccgaacgccgtcatcgatgtaacgatatttgtttggatcgt
gctgaacggccgtaatagtgaaaccgcctgtactgtcgtcgttagccgcgattgaggtgaccctgaagtactgtattgcgaggttatcactgtcta
tcgcccaaacagcgcccgccacaggaacctgactgaatgccgtagccaccgtcaccgtttttttatcggcgctcaccgcgctgattgtccgcgt
ctgggcttttccgtcgggaaggttaaccaccagccggtctttcgccgcgtagtctatttctcgatcgagggtaatttggcggccgttggccgcgct
tatacggcccccgttctccttaccagagcggaaaggatcggcgacaccgataatttcagcgggcaaagggatataaccgtccagccccacgc
caaacgatacggtcccgtctttggcattggagagcaatacccagcgaccgcgtcggtgcgcttcactttgcgaggtgcagccgattgcggtca
gggacgtctgccggacgtcgtaacgttctacaagcgccgaatcgtaaaccccctcaacggtatcgctgtaatggttctgcggatcggaccagg
acaccaggcaggagctgtagcgattcttgtatgagccgcccgcataagtaaacagcccatcgataacgtttgagacgttataaacccagtcaac
atcgtcctgcgggacgtctgcctggacataaatctgatcgtttccccagaacgttattccacgaaataccgcggcgagatcgttaagtacctgcc
aggcgtcctcctggctctgaatgaaaacgttgcaggtgaaacgcggttcggtgccaccggccccgtcggaaaccatttcgtcacagtactggg
cgattgaatacagcgcccacttatccaccatggacgcatccacgcgcgtgcccatgccgtaaatttcatccagaaccagatcgtaaaagatcca
ggcagggttattggaccatgccattttgaacccgccggaccatgaaccagaataggttcgggttttcggatcgtaattatccggaaccttaatcag
cttgccttttatcttacaggtcactttcggcgcgctgccgttgaattggctgctgtccacttcgacatacaggagcgctgttaaaggataacgaagc
ttgctgtcgatgacttccgcatacgaaaacaccttgaaggcgttaaccagtttcgaatttgatccgctggcatcagccgtaatacgcctgaccctg
acagaccagccggacgtggattttggcagatcgatacggtggtcacgctgatattccgtcgtggtctttccgtcaaacttgccgtttacaaccgttt
tccaggcgccgccgtccgttgataaatcgatcgcatactcggtgaccgtgcccaccatatcgccattatctttatagagatactggaccggaagg
ctgagcttgatacggatggcatccagggaaaggtttgtaaactggcgcgtccagggcgcggtggtggtgacagttgtgcccacggccagctc
gttgtcgacctggggcatcccggcaatataggtctggtcctgtgtgcccttgcggaactcccatttcacgccgctgaagttgtattccccgctgtc
gtttgccagcggcgtatcgttgagaaaaatgttctgagcggtcaggtcgccctgtatttccccctcagaaacggcaatgagcatttttaattttgcg
accgacagcagatcgtcaggctgctcaaccggagtatgtgaactgccacctccccctttggcaccctgcaggatggtttcttgtttaagaagctg
cattttttcacccataaaaaaaggtgccgaagcacctttaagttagtggccgctggcctactgctgatcgctcgagtacataccggcgctgactat
cgctccccctgcctcagtcagaccgtaggccagggggacaggatgccccatagcgacggtattgaccggcgccccgaaggcgtagttaggc
gtgttgtccgtgctggaggatttacccgcgccgaaggatggctggggcgtgagcatctggacaacgccccccagcatcatcgacactccgac
ccctgtcagaattgacgtggcgctgatagctgttgcactcatcgccgcgccccaggctgccatgctcgcaccagcggtaaagaatgcagcgac
cagcgcaacagccccgacaactatctgcaggacgcccgaacttttggccccctcataaacgggcacgatccggtacacgcttccaccgcggg
tcatatcaaactcttccagcccgatattgttgccaccgttaaaaaaggcgaaacggatccccttcatatgagcttcagacatatattttttgaatccg
ggaacctgtgaacacatggccctgagcatctcgcgcaggtcggcaacatcaaactgaacgcgtttaccgaatttttttgccattttaccttcgaga
ataagcgtcttaaccatgcattctgtccttatgcctgaccacccggaccgttctgtcgcgataatattttccataaggcgttcgcgaagaaaggtgc
ccgaaaagatgatggagaatgatgttatcacccacatataccgcggcgtgattagtcaccgatgcctgcacactcatcatgatgatatctccggg
ctgcattgcaccggcggcaatctcaacgaatccctcacgctcccagttgtcgtcgtagagacgctccttgccgctctcccaccattcgtaaggta
ctgaataattgccgagaacaatgccgtattcgcgcagataaaattcacggataagcgaccagcagtcggcgtaacccagcacccactgccgcc
cggcataatcccggtcttcacgcggggaaatcgtacaaaaatccccgtccggccaggacatgatcccccactcaatccccgaccagtcgcact
ggatccggtccagctctgagggcaccagccgaaccacatccggatgggaatgaatgagcatgatgatctcaccgcgcgcgcgggcagcga
gctggtcttccggggagagcgtgaatgtctcctcgggtttatcggcaatgttgcggcagggaataaagatttgttgctggcctgactgaacaatc
aggccgcaggcttctttggggtattcagcagcgacgtgctgacggatagcatccagcaatttttcacgcatttttatttcccctgcaggtttgcagc
cggaaaaccgccgaacggcagcggcgcatccgggccgtgacgatcctgacaatcctgccggcggccgccacaaacatctttcgacgggtc
atcggtcggtgtaccgtctttggtaaagtatttcgtgccgttgtaatcgcatccggtcccgcttcggtaccagccccgcatacaccaggtgcagac
aggcgtaatctgccgtgtcggcagctgcaggctctgaatatcgaaaggagaacacagctcgaaatcaacctgtacccgcgtctctgcggtttta
gcattgacgtaaaagagctgtaagcgctcatcggccgggctggcacccggattaccgtttttccagttggcggcatcgagatacttcgaaagcg
tggtatggattttgaccttagccctgaccatatcgtcatattcaagacacagcgcggtgacatagtttccgacgttcccgacggacagcgtgggc
gttggctgggaacctgtactcgataactccatccccttaagttcgtagggatggggatcgtactggtttccctgccagataatggcgggcagatttt
ctgcggcgaaggctgcccacccctcttcctgaatattgtgcgcatgaaaacgcagcacctgatccataccgaattcagtgccgtcgatctcaatc
agctgaataacgctgccgggctcaagctgttgtatgtctgccgtaaaactcatactccccccataaaaaaaagccgcccggaggcagctttcag
tgtttttcgagaaaatcagggcgcgaacgcctgttcaaaagtgaaggccacagtggcttttttcccggtagggaaagaaacgctgaacgaatcg
gccttcattctgaacagctttttttcaccccatggagtggtccaccagaacgatttagtaacgtgagacatcaggaaagcgcgcagcgcagccgc
ctcctgtctggtgcccgtccagtccaggttccacgtttcctgtttgtcgttgatccccatccccgctatctgtttgtagccatccccgaactgggcct
gcagcgttcgggctgtttcagtgccctgcgctgtttttcgcgtgcgccaggtaaacgtgtccgtcactgtgtcctcctcgaataaagcacgccgcc
cgcggacatttcttttttcagtcgctcggtgattgtctgctgaacaatcgcctgcagctgtttcgccgtccccgtggcgttcgcctgatttatgcttcc
gtcactcccctgctggctgatgctgactggggcataaacactgatcccgcccatgccagcaccggctgcgttcccgccgccgaccagaccac
ccgaggcatacccgcgcatcaggcgatagagattagccacgccgatgcggctggttgattctttggtgaagacgaattccccgcggtgaacga
taccggctggctcgtacttgccgccgtgcccggtaaaaccgcccacgtcaaaaccctgtggccggtatgacgggaccgcgaatgactgaccg
gcagaggaggttttcgccccgccgctaacccagcccattgcactctggatggtgtaagccaccagcagctggttgataacggacacaatcattt
taaggatcgagctggtgaattccctgaagctcgccttcccggttgtcgtcaggctggtaagctggcccgccaacccgctgaacgtagcctgaga
aatctgctgaacggagctgaaaacgtttgtcgctgaatcctgatattcggcccaaccctgtttcgcaccggccagccagtttgcacgcagggcat
cttcagcttcgaacgtcgccctttgctcttccagaaccttttgctgcgcctgagggttgtacgaatagctttcgctgagacgctgcagcgtagtttgt
cgcccggcttcccgggtggataacccctcagactgagcctgcaggcccgccctggcggctttttgctgctgctcaaacttcacggcctgatcgg
ccagctggttgagcttttgctggctggcaaccttatcgcccaggtcggccagctgccgcttgtactcgagcgtttcttctttgtgcgccagcaggg
atttttcctgcgccgtaagctgacgacgcccagcggcctcctgcagaacggtgaactgattttcagtttgccagagatcctgacgctgtttacttat
gacgtcgttcacgctggtatgctgctcaagcgttttaagctgggcctgaagggtgagaagttcggcctgcgccttttcctcggctttgtccccggc
gggcgttgagtagcttttgcctttcggtgtttttggatccttccactgcttttcaatcccggcgcgggccgcggcaatgtccttttcagtccacagcgt
ggcgacaccgtctttcgcatcctggcggtttttctcaataagctgactgagctttttctctgctgaagcccgcttttctgccgccgtcgcgccggact
ccaccagctggttaaactgctgctggctgcggattgcctgagcctgctggtccgttcgcattttttcccgcgcggctgccagcccttcctgggcgt
attgctgatcggcaagatcgtaagcctgctttttcagctccacctgctggcgcgcgtttctcagcctttccgcatccgctttctgcagaacgttgtta
ccggcataatccgggtcgaccttaagattgctggacagcgcgcggtactctttctctgctgcctgccactcagcaaaagagtcctggcgcttcat
cgcggtgtcaggattacgcccgacgcccagcatcgcatcccacgcaccggaggcggcattcttcacccagttccaggctttttcgagggatcc
gagattatcctcgaccgcaccggcgcgctgaatgaccgcgtcggaatatgcccgcatggccagctcggcagccttctgagaatcccccagcg
cctgagcagaagctatctgttcatactgggtggctgtcagaaaatgaagggaatcgttgagcgtcgcgaccgcgttaaccggatcatccttcag
gcgtttaaactgatttatggtttcgtcaacggcctgcccggtagcctgctgcagcctggcggcaacattgctgaccatgctgacgtcattaccgct
gaacgcgccgctgccaacgacctgcgccagcacgcctgcagcggcatgctgagtgatgccattacctgccagcgagcgcgccagcgcctg
cagctgccctgacgttttccccgcgtagttcccggtcaggatcagctgcctgttaaattcctcagactctttgctgccgtcgtaccaggccttaccc
aacccgaataccgccgcggcaatccctccgaccatgctggcgatcccaagaccgcgcagtgacagaagctggtctatccaccctgcccggtt
agccagcgtgatcccggagccgcgcagcgcgccgaagttaccgcgcatgacctcgccgatcagtattcccagttcctgccgggcggcggca
ctttgcagccccagaccgtgcgtggcgactttggcagcttcgagcttgcggatatagacttcagccgcatcgctggcaccgacctgcgccgcct
tcatgcgcagtagctcggtaccggagagcttttgctctgcaacctgttgcttcagctggctgaggaatcgcgtgcgcgctgcggccgatttttcct
ccacgatctgcagttctttttgacgggccgtggtgcgggaaataagggcgagataatcctgctgggttatgttgccctgtgccctcgctgcgcga
aagcgcgcctgcacgttcgcaagcgactgtgtttcaccattgagctggcgtacgccgtcgatctggcggaaaaatgatgccgcaagttcatcct
gtcgacgggcaagcgcagcggcctgcccgtcattctcacgcatgcgctgattaagctcggtcacgcggcggtgagtttcatcaacggactttg
aaacgttctgccagtctttggtaagcccttccgttgcggccgactggcgggatttcatatctgcggcagccgccgcgccagcgtcacccacggt
tttaaacgcagccgcctgccgctctgaagcgcgctgcattcgcgtctggactttttcagagtcctcagccatcccggttagctggccctttatgcg
ggcaacctgctcactaaacgtggcgctgtcgacgtcaaggttgatgaccagatcgctaatctgctgggccatatcggatacctcctgttatcccct
cagctgcggccatcagcgcatcatcatccggctcgtcatcgctgatgacgataccggaaggagaaagcaggctgaaatgtgcgggggtaagt
tccgggtcgcggaagaaaagagtggagatggaataaagcagctctgagaaatgcgcatcgagctgagcgtcctgaaaataatgctcccggta
gaactggtgccagtcgcccagctcagtggaagtcattccagccagcatggcgcgccagtcgggtcgcccgaactcgcgcgccagattcagg
acaaacttcagctcgctggcaagggcttttccgccgcaacgggttctgcgctttcggcctccgctggggcatccggatcggcagctttgtcatcc
tcaaccggaacgagcatgccggagagcagctttatttccatttctgctttaccgatcgcctccggcggccagccgctaagcacctgctggtaaa
gcgtctccacatccgtgccagccggatcgttatgccacaaagacatcgcaatcaaacgcgcaccgcagcgaatatttgagccaatcagcctgg
ccgtcatttcctgatcgctgatgccgtcgctgtcagcgctgacggccttttcctctgcggccataaacgtgatgtactcaatacgctgaagcgccg
acagctcgaagatggtcagtgattctttttgccaggtgaacttctcttttttcagaaacatgcgtccttccttacgctgcagttacggtaactttgcaga
ccgcaacgaaattaccgtcgctggtcataacaataacgtcagcggtgcctgccgccacgccggtgacggtgatcgcattaccgctaacggtga
ccgttgcttttgccccgtctgaggttgccacacggaacgaggtatctgaggcactggctgggttaaccgtcacattgagcgttgtggttgcgccg
acggccacgcttgccgtggctttatcgagcgtaacgcctgtcacggggatattcggggtcccgctttcttctgccagttccggcttgccggtattg
gtaattttcgctgtacgggttatgacctcttttgccggaatggctttacccaggctgctgcaccagccgcggaaaacgtcgacggtaccgttcgg
gtatttgattttgtaatagcgtactgagccatcaataaaccatgcgacaaggtctttttgcccttcttcgcccggcttccaggcgagggtgaacgag
gtatcgccagcagattttgccccctgggccgtcgcgttccagtcggcatcctcgtcgtcgaggtaagtgtcgtcatacgattcggcggtcatttcg
cccggcgtcagctctttaattttcgccaggcggttccagtcgatatccgagagtgggttagcgaaagcgttgcccgttccggtgtaaagccagag
ggtggtaccggcacctttcacaggggccagcgggtttggagtaggcataagtacctcttaaattgaataggtgattaagtacgtgaaatcgactg
aaccccaggtggccatttcatcatcccgctgatagtcataaccctgcggggtgaacgtctcgaccagttcggtcagacctgggatgaaggccat
tgccggatacactttctcttccatccaggaatcaagcgcgctgtcggggctggaggctttaagaaatacctcgatgtgaacaaccgcctgccac
gaatcttcgtcaagcgattacacgtcttgagcgattgtgtaggctggagctgcttcgaagttcctatactttctagagaataggaacttcggaatag
gaactaaggaggatattcatatggaccatggctaattcccatgtcagtccagtgattctgaataggcgataagtccggtataaccggggataatct
caccattatcagcttcaaattcaggaattgtgccggtggtgatggtgtattgaggctggccatcttccttcgcgaaggctgccaggtcttcaatctg
cttagctgtaagaactactgtcatgctcattcctcagttgtaaaaaagccccgcgagtgcgaggcgatttgattgaattctcggctcttatctcagcg
cagccccttactgcgtgccggttgctcggtgatgagcatcagcgatgagacattaaagccgaccgaaggccagcggcgttcctcatgttgccg
acagagccatatcgacaagaggacgaaaactagcagcatgaatcgcctattggttattcgacagtcgcactgattcgtaaatccgctcacacgtc
attcctgcccggtagctttcgtcagatcgtccagcataatatcgagctgcttctgcaaggcttccgagcatgtcggcaagcattgctgcgttggctc
cggctgttttgcttctgacggaagtggcgagatctgcggtgtgctttgcggcgtccatgtgggtagcgagttttgttgcttcggcgcgcagctgctt
aacagtggtagccaggccagcagaagtaacggcagcgctcgctgcttgagcttgagcatctttaacggcctcatcccgggcgattgttcgccct
tgttcaatcatacgagctgcggtctgtgcattcgcttcctgtgaagattccgcgctatcccggtcagcccactttattttccagctgcggttcgtcca
ctcactgccagcgagaaacgaacctaccaacgcaacaatcacaatgatgcagatgccacccggcttcactggtctatcccccagcacgtcagt
gcgctttcctggtcccgccgttctacctgcccataacatccatccttctggcccttggtcaggcggcaatcgcggccaccgtctttaatccaccag
cggatagcttcacaggctcctttcgtatcgccagcattaattcgcttatagaacgtagacgggaaacattttccggggccgatgttatatgggcag
aaagaagcgatacccgctttctgtggttcggtcagtggtactttgatatttcggtcaacccacgccagcgccttgtcgcgttcaatggcgtttacct
gggcgcatttctcagctgacagcttcatgccctgtactactggcttgccatcaaccattgttgcaccacggcaaatggtccagagtccgccgccg
tcgcgatatgctgtcaagctgttaccctctttctcatccagaaactgatcgagaatcacgggtgcggaagccccggcaagaatcaaaccaacga
ccgctgcgctcaatttattcttcagctttagagacatagccattgcgccgatcctcccgttctttccagcggaaataccagttcactgcacaggtgat
taccgtgcatgcgataccgacaataattgcccagtcgctcaggcttaaccctgcaattctgtcggccaacatccaggacacctcttttgctgtttta
gctgtttcggcatatgccttcgctgatacaccgcagccggcaagcgtggttcctgatccatatgaaagtctgctgtaaatggtgctcattctggtca
tagcctcacctccgatagttcggatggcgctgtgtgtgattgaaggggatcaggcaaccgggctcttatgttcaagtaaaaattaaggatgattcc
cggtgcctgaagatggtgatcaccacagcaacgggggagcgtggtgatcgttatgattttttcagtttttccacctcttcggtggtctgtataaacct
gtctgcctccagttctacgccgatcgcccgacggccaagttctattgcagctttcacagttgaaccagagcccataaagaaatcggcaacgatat
cccccggtctgctgctggcgctaatgatctgtttcagcatgtcggcaggtttttcgcatggatgtttgcctggataaaactgaacaggcttatgtgtc
catacgtcggtataaggaacaagagcggaaacagagaagcagcgccgaaggattttgtattcctccagcaattctgaatacttgcggtttaatga
ctggtaggtagccaccagctggtggtgaggatgttcaagcttttgctgaatatgtttatcgatggcgatccgcgtgaacagttcctgcaattttcgat
agtccacttcattcggtagttgccattggcttgcaccaaaccagtgtgacgccatgtttttctttccggttgcctcagctatttctttcgagctgacacc
cagtgattcacgggcattacggaagtaatcaatcagcggcgtcataatgtgctgctttagctctgtgcttttcctttcgtaaacatcctctttacctgta
tacggtccaagatagtgctcagcaaacaaaatccgttccgtagatggaaagtacgcacgcaggctttctttattacatccattccagcggcccgat
ggttttgcccaaatgatgtgattcaaaacgttgaatcgggcgcgcatcataatctctatatctgaggccagtcggtgaccgcaaaacaggtagat
gctgccagcaggtttaagaacgcgagcatactcagccaggcagctatcaagccagcgtaagtagtcctcgtcccccttccattggttgtcccag
ccgttgggcttcactttgaagtacggaggatccgtaactataagatcaatagagttatccgggagggtggcgacgtaatgcagactatcagcgtt
gattaactcaacactgtttatttttacagtatttttcatagatcagtaagcgtaactctgataggctcacgttgcttttgcgctaaagcagtgggccttg
gttagcttgtgacctgaaagcatgagctgatggctggccgggtgcgctaacacccaccagccgcccatttccacagcagaaaacccccattact
ggaggcgtttataacatccgaactggtaatcagataaccccgccatcaccagctgcgtaagtatgagctggcaacgttcgtggctgaggtgggt
attctgtgcaatctccccagccgttgctggtttatcgcttaattcattgaaaacagcctttgccgtttctgtcatatcttcctgatttagcatgtcttttacc
taaaattagttgcgtgacatacagataactctggttggtgataccagcaagagaagaatttgattctgcaaccaacaaggcctttaggcatcaggc
aggaatgagatgcaataaaaaaaccacccgaaggtggtcttatatgaatctttaacgcggacttagcaaatattccacatcatcgtactaccgttat
ggttttcgataatttttgcggctgggctagtaccaaaagagtgcatatagcaatgatgaatagtaaggaccagatcctgcaacgtttggtcactctc
tagctccatgatatttaaaccaatattttgagctttgtccaaatgaatatgtctggcatgtgcatacgttgcttggtggttgtttaactcatcacatatac
gcttagccttagcttcagcgtcagcctgacctgcgaacataccagtacaaagccatttctggacaatttcgttcgcccagagaattgctttttcaca
ctcgccaatcaacgttggatttagtttttggaacgtaaattgccaccattgcagtgcagcagggttggcaaaaatttccgcttttgctctctcatactc
ctcaataattgcatgagatgataacccattaaactgtggatcaattggccccaagttcgactgtttacctaaaacgatctgctcagcacaacaagca
agcattgtgccacaactcattgaaatcataggtacaatcgctcggatattggttccgaactttgaacgaagataatgaccaattgattctagagctg
cgatatcgcctccaggagtatggagtaagatatccaatcccagactcgtatctaacccattgatagcagacataagaccatttttatcatcatctga
catctggatcagatgttgaaacccaggcccccctttttgaaggaagcctgagtaataagaaattacatttcggccagtatgtttcgataaatcacgt
aagtacttgtggcgaacctcatccgctggtgtacgttgagcgatagtacccatctcacccaatacgtctatccaatttggcatgttatcaatttatca
gtatgagtacagttggtgagattgctgaccgttctgctcagtagtatttggtgttactgtgctgtatgaatagagcacaccacttctcacattcagatc
gttttgctgagcgagaacacgcatagcaaaatgctgtacggattcgcctttttgaaactcttggggttgtatgcccattttttcgtaaaattcagcagc
gctcatgatatgtccctcgtttttttctacatctatgcaattccaggagccatcaacacaagatgtagtagttagcagtcgtcaaatacacgaaaagc
ctcaagatgaggcttaaaaagattctttttgataaagatttagccaaactatagcggtcaaaatgcagatttgacaagtataaaaagcacttaaagc
ctataaataggcagtttttgagaattaaagcatctttaatgaggttgaacaaaatgcagtcttgacgctgaacaggactttactggaacgtagagct
aaatggttcgatttcatgaaccagttacaaaaaaacccgctcatcggcgggtttataaaactttggcaacatatcaaatatgcttcaaatatggctta
ttttgttgcattttgcaagcgtgtttgaaggagatggtgaaatttacttcacatttctgccactttgagggcttcttcttcctcatagtattcaagagccat
ggccaacgcagattcatcaagctgggtaaaagcggcctttaacccagcccagtgccctgaatagacacgcaaccatgtcgaacggtcaacgc
taaccatgcgggccaacgctgcaccagcatagtctttataggtttcattatttctggttgcggcaatttcctgccctgccagccataccaggcctatc
agtttctttactacgcgctcctgaagggagttatcacccaggcatttctgataagttttccagacgtattcacacatcatcacctggtgcttatagcta
aggtcaaaaccgtagcagtaccgcaaccaggcctgctggtatccactaagcgcggacactgccctacgccacggcgcggactcaaattccgc
atcttttatcggcggcattggcctgcggcggctgcgtgtttccagcacatacagtggcgcggaaagtgagttaacaaagcgtggccccttctctc
cttcgagttcgacgagatgaattccacggcgaggggtggcatttttgtctgctggtgggtgttcactgaaagcctcaagctgcccttttgttccccc
agacaggtcaggtagcgcgcggcgcaattctattcttacaaaattcaggtcttgttgattcatgcttctttgcgctccatacacttaagctttcgcaat
tacgccgatcgccagcgcccgatccataaaacgcagtagcagctcaagctgcgtaccatgcttctgctcgaatgccggtacatcggcgtgtaa
ctcgtcgtggcactctctgcacagagggatcacgaagagatcatgggcttttgttgctgtcccccccataccgtgccctacgatatggtgcggat
catctgctggccgtcggcaacactcacagggttgtgttttaacccagcgggtgtacgtctcatttatccagcggcgtcgctttggcctgagcatga
aagattctggagactccggatcaacagagagcgtgaggatcttcttcgccttctcctgcacgaggctggttgcagaagcggaaggcacaatgtc
gctttccctcatgaccgagcggatcttatcatccggaaggcgtagccccttgtgcgcaacgctttccggaataacatcagccaggtcgtttctgac
catccaccagcacagttccggaagcgtcaggatatgcgactcgggaaaaccagaatcacgccgaatgacttccagaatccaggataccaggt
ttcctgccgctatacctgcaagctgttcggtatgctgccccgacaaagtgtgatcgcaatgccagcacaggcgaatacttcctggtgggtgccgc
attgttgtgaagttcttgtcgtgccacgatgaatgtggccactggcattcaaaccgattactcaaccattgctcaagggaaggaagcccaccagc
acgctgaataacccgatcattctcgaagacctgccgcattaccggatcatcagccagcggctgaatggcggcgggaacagctcctgtactgaa
tgacgccatttcttctggttcaggctcaagcagaacgcgaccgcgcataaagaggtgcatcagttccgcgccgggacgaaacaacacaatccc
catacgatgggcgatctcgggggtaagcagagctctcacgcgacctgccccctggcaatgtgttctgcccacagtccaccaatccagcgcac
gcctttcgccgtgaaacgtgcctggctgaatgcatgatttgaggttacggatgtgccggttttcacttcaaaacggcccgcatcaatatgctgatg
ccgtggggtcatcgttccgccaagacgatacatgatgtcgttctcaaggaggaataaccgcagatcgggctctttggccttaagcagttttgcca
cctggcggaatgacattgacccactggctgtacagtaccgatcaacaaacgctaccttcggcgccgcggcagccagttcgttagtcaactgctg
tttttgttctgcaaggtcagctgcaagacgtagggcttcagagaatgattgaggaatcgtctgctgctgtgcctgctcaagctcctgccagcgatc
aaccagacgcgcggtaaactccggcgacagctgcgcgacaacgatataactgtcccgcttccctatcagataaaccgataccgactgattgag
gtgatttttaacttcccccattggggggagttcaataacaccgcgctctgccaggcgttcaatggaccgtttaacatggtcatgtcttgattccacca
gctcagcaatatcgctgctggacatggttaacgctgttgttgctaactggctcatacttttctccatatcaggcggctgcacccgccggttcatatct
gctgattgttatctctacccgacctttcggcacaacgggtccccattccaccagcatgcgcttaatctggctgtcgtcttcccagacacccgcatgc
gtcagcgcgtcaaacagggctttgttgtaattatcgatatcccggcggcgcgcatccggcgggtacagagtgatttctaccgctgccagttcagt
cgatggcttcgggagacgtcgtaattgctcaatgatcgccacgcaggcagcgctctggtatttacggccagcggcgctaatgaggtgacgacc
ggccagcggccccttgttaggggcgcgccagtaagtgttcacgctcggaggaaaaggcaggatcagtttcacgcggcctctccccgcatattg
cgaacaagttcagaagcagcagtaatgatttcgctggtggcagtccgctccagccagagttgattgatattggctttcagcttgtgttgcagtgact
catccagcatgtcagcaccatccacctggtcgaatacaattctaacctccagcggccagatacgggactcgggaagcggatccgatactggttt
agctttctcacgaatgtgcatgcggatctggcgaatattggaccaactggaaacatccaggcttcccatggctgcaatgaagtcagtgctgttcat
gccatattcaccggatgcttcaagggcaacagtgcgaatacgttccgacatatccaggcgcacagcagcgtcatcgaattcaatcgacaacag
ccactcatccacaccgaacaaaatactctcacgaataagcagcttcgctttgtcgatcgttaaaggtgatacctgagtgaattccggtgcttcgac
agaatccgccgcccaggtatgcccaaacttcgattcactgaatgtgtattcttctttatcgccaaacgcagctctaacgcatgcccacgcctcgac
accgctgatagcaaaaatatctttctgggtgagtggcaactctgcttctggcttatcagctgcaggaggtgtggcagttacaggttgagacttgct
ggcagcaaattgtgccaaagccataaacgcccgcccttttgcctccagttctgtgcggttgatatagctgaaccgctcgccacgccatgacttatc
gaatacagctatggcaccggcaaaaaacgcgctggtgggtttctgtttttcgtcagcaggtacaaaccacacaggcagatcgaacccaatgcg
cccgcgaatgaatacaatgtgatcggcatcttccggccaccacgtttcactcggcgcggcttttatcaggaatacatagcgaccgcccttctcgc
gctgggctgctgcgtagttcatgatgtgcgtcatgccggtgatcgcctgcttctcgtggtactgcgaacggctatacggtgggttgccatagcca
gcgccacccagttcagccagacgttcagaccagtcctgcgtcagcgcgttatcttcggcggtgtaccatgccgggcatttcgcgttgtcgtcgtc
agcaaacaaatccagaactaatggaccaaatagcgcgttgatcccccaaaaaagcagatccggtgtccgccactgatcgccaacctctttcaat
tcgtgagctggtttgctacgcagtgccgccagcgcctggcaatatttattggtcatcatgaacggaaccccgaattttctggcagtgagtaatcaa
cactctggaagtttgcgcggctggctgagttagtctcccatttgccgttaacgcgttcaggccggccagcactggaccatttggtcgcgctttgca
ggtaaccagggaagttttttggaatgaacagagttgccgggcggaggtattgcgcctgctcgctatcacgccaatcggcatttttgtaatccacta
ccaagcacaggtcatcaacagtgaattgttcccgaagacgggcgcgaatattctccagcgacgtgctgcatacctggtagcgtgagccagtag
tctgattcaggtaagacaaaacctgtctggcctgatcagtaatcacaacctcagggtcgggttgcgccgcaaccggacaagagggttttgaagtt
acttgtggttcttgttttgattttactgacggatccccaccagattctgacgggtcaaaaccgccttttttgctggatttcgacgcctcaaattttgacg
ggtcggattttgatgcatcagattttgatgggtcagattttgatgcgtcagattctgacaggtgagaaaaagcagcctctcgcaacttaacgacgtt
aagctgatatacgttcgatgcgttacggttcccattacggcgctgcttacgggaaagccagccatccttctcaagctgagcaagggccgtcctta
ccgtactttctccagcaccgatttgacgtgcgatcgtgccaatagacggccagctaacaccctcatcactactgaagtcggccagacgcgccat
gatggcaacgctggataacttcatgcctgacgaagcgcatgcatcccatacgtaaccggttaatttagtgctcatggtcgtcctttaattctgtaaat
ttacgctggaattgttcaagagggctgaagcactcatgatcgtacccttcgcgaaggtatataacgcgctgtgtatctggctcccagcggacaac
tctgacgggaactccgtagtgatctctgaaccgccggttaacttcagccattcctcgcgccccttctcgttcatctgaacaaatgcttctaccatcaa
gtctgctggctggtagttgcctccatcagccgcgttatttatgatttccacatagccgaactgggcatctttacccaccagcggcaaacatctgaat
tgcttagctggtctgaatcggtttacactgttcatgcgttagtttctccactgatacgacacgccaaggcgcccggagctgcacactcgcgggcgt
caccttttctgcctgttgaaacgaatacgtcaatcgcctgatctgaaacaccaaccccataaagcgccataaatcccaggaacccgtgaatctgg
tggcggagcttcttactgaataattctgaaagcattttgcgctctgatgaatcaattaccccatcagccgctgctgccatcttggcattagccagctc
accagatgctgctgccgctttcatctcaatgctgtacagctcaacgttatccaggctctcagcagttggaacatccaccagccatttcccttttcggt
tcgcctggtactcggccaagtaacaagaaccagacaggtcctccatccgttccagttctgccaaggtaaagaaccgactgccacacttctggta
caggtggttgtggaactggtcgatagtcatccctaaatcggaagccatacctaagcgaccatgcttgtgtgccttacacatcaggcggattgctgt
atttatgctgtctaccatgttgatttccctctggtagttaataatcaacttaaagttgactattgttgttagcggaaggtatgccgtcatttttgttcggata
aatatcaggtcgtaattgatggggagttactacccatccgccccattggcagagttgaataactctttcagaaggtactcggttctttgcaatccagt
tcgcaacagattgaactgattggaattcaaaccgccttgatacctctgaaatcgacccgatcgccttcacagctttagctgttacattcttgtgttga
gatgacatgtgttctcctatgactaagcctgcatcaatactacttatagtagcaattattagcaacttaaaatagaaatgacaactatgccttgtgcgc
ttaatcttctacttatggtggaaaatgctaaatacaaagactttgccgaaaggctaaacaggtctctccaagagcaatctattggagttaaagaattg
tcagagttcagtggtgtctcgtatgagatggcgcggcgctacactcttggtactgcaaagccgagagatgagaagatgattcgaattgcagaaa
gacttgccgtctcaccggcttatcttgattatggtgtgcctgttaatggtggcgacgcgccagccaaaggcacggtcagaatagagcaattggat
gttcatgcttcagccggttccggatatataaaccaaccattccctacaatagtgagctcaatagagattccagaagagaggatcttcgagttgtttg
gtcgtagaagccttgatggcatcgtcatgataaatgttgatggcgatagcatgatgcccacgctttgcccaaaggacctgcttttcatagacagca
aggttgaacaattcagcggcgacggcgtttatgtgttcaattttgaagacagtacgttcgttaaacgtttgcagaaggtaaaagggcgccgactg
gcagttctttcagacaatgaacattacccgcccttcttcatagaggagcatgaaatgaatgaactatacatattcggcaagctaatcagatgcttac
ctctaaaaatgatagagtttggctaataattaattcatcaagaaaccggcgaaagccggttttttttacgcctccaattcctcacctcataacactaca
ctactaaaaatttcattttctactttttgttgttgcaattatctacttaaagtagctatagtcattgcatcgaaagcgaacaggcaggacgcccacgaa
gtagccgccggtggcatatgaataaccggatgattcgctgacagaaaacttaggttgggggtagaggtttacatgaatcatttattcacatgctca
ttttgcggagcaaccgaactgggagcgataaagatcgtcgcaaaaggtggtaaggacgaacctgccatctgttcggaatgcgtagtcacatgt
gtagaaaaaatgatcctgactaaaaaatcagaggctgaaaaaccaacctctgataacgaaataatatcagtcgataaaaaactatttaaagagctt
cttcagcttgtcctcaaccttcctgatttcggaagtaagctggctgctgttgacattgatagtagctccacatcgacaagtgaaacttttgttcgactt
gagccaagcgattttcttcttcgtcttagtgccgcacttagggcatgcgggtaacgtaatttcctggttatcaaaagcgcccataaacatccctcttg
gttgtgtgagaacaccaagataccaccgcgcctgatgtggttaaaagcaggctaaagcaataacaagtaactccctgttctggcggcccggtgt
tttcccgtgtatttccggtaaccgccagcctttttcagggcacaacagaaaagggcatcaccgggcgacgggctcataacccaatccacccgg
gcaaaaagaaagcggtctctgcaagccgccgaccaatgcaggtgcccttctctgttgtgtatggagaaactaactttttagcgtctgtgcagatg
cgctgaggaaccgagaatgaataatccgtttttcaaaaatatgttggtgtatcgcattagtcgcgatttcaccatcaaccaggaagagctggaaca
gcagcttgaactatttcgcttcactccatgcggtagccaggatatggcaaaaaccggttgggtatcaccacttggtcagctgtcagatcgcttgca
tcacactgtcaataatcaagtgttgttggttattcgccgggaagaaaaaatactgccatctcctgtcattactgaagaactgcgcaagcgtgtgtcg
cgtctagaatccgatcaggggcgtcgcctcaaaaaaactgagaaagattcgctgcgtgatgaagtgttgcactccctgcttcctcgggcgttctc
caaaaactcgactgttggtttgtggatcaacgtcaccgacggtctgatcatggttgatgcagccagcgctaaacgtgccgaagactcactggcc
ctgcttcgtaaaactctcggttctctcccggtggtaccgctgactatggaaacgccgatcgaactaactatgaccgactgggttcgttccggtagt
gcgcctgctggctttggcctgggtgatgaagccgaactgaaagctattcttgaagatggcggtattggacgctttaaaaaacagactctggtcag
tgacgaaattcatgtgcatctggaagctggcaaagtagttacaaagctgtctatcgactggcaacagcgcattcagttcgttctttgcgatgacgg
cagcatcaaacgccttaagttctctaatgagattacagaacaaaacgacgatatcgaccgtgaggatgcggctcagcggttcgacgctgactttg
ttctgatgaccggcgagcttatctctctcattaacggattaacaacctctctcggcggcgaagccaagcgataaacaccaggcaacaattacccc
cataagcatgggttgggttgctgcacgctaaattcagcaattcattaatttaatggcgcggtgcagcgcgccaatatggagaaaaccatgagcta
cattcagacattatccggcaaacattttaattacctcgatatccaacaggacgatatcgtgatcgaggatattgctaccgcgttgtctcatatctgcc
gctttgcagggcatcttcctgagttttacagtgtcggccagcatagcgttttaaccagccacctcgttccgcaggagtttgcattagaagcactgct
tcatgatgctgctgaagcctacctgcaggacatcccctccccacttaagcgcctgcttccggattaccaggcaatcgaagctcgtgtggacgca
gccattcggcagaagttcggtctaccaactgagcaacacccaaccgtgaaatatgccgacctggtgatgctcgccagcgaacgccgcgatttt
gagattgacgaaggttccatttggccatgcctcgagggagttgtcccaacggatttattcattatcaacccagttcgtcctggccagtcatacggc
atgttcatcaatcgctttaacgagttgatggagcagcgccaatgcgccgcatgaaggtaaaagaactcgtagcggaggcgtttgcctccgttgct
gaattgccaccaaagcatgcgccgcttatgcgcgaagtcgccaccagactggacgctacgttcgcagcattaaaagagtctctggtgcaactg
gaacaggaacgtaaagataaaacgccatgaccgtatttgaatatctccaggctcatccgaataccaccagcggtgaaatcgccaaaggtatga
acaaaaagaccccagcggtcgccggagcattatctcagctctatggcaccggtcggatcgtgaagtctggtgttcgcaagggtattccaacata
ccgcattaacgatatgccgtttggttgcagtaacagcctaaccatgatgtttaaccagctcttgagcagagccagacaaggagcagcccaatga
cagcactcaacaaacaggcgctgcgtgaagaattccagttcatgcaggacaactatagcgacccggcagaccacgatcggcaggtgatttac
atcgaggcggaggcgctgctggatgagttggaagccaaagactcaacgatagcagcacaacaacatgagatccgtatgttgctgaatgcgctt
gaggaaaaaccatgcccgaaatgcaacgacacaggaatgactgatagtggcggcacgcagccatggggcgagccgattgagattgaatgc
gactgccgacagcaggatgccaacaccgcagaacttgtagccgctggcattggcgtgaagggggagtgagatggataaattaatcaaaccta
ccgccaaaggtaaatatgacggttcatgtgattatctttgctcggaagatgcgcgattcatcgttatgcgcggcgattatacggaagcggaaataa
ttcaggcttctgtgtctcaagatgtaatcgactcggatggtgcggctgattttgcaagtagcgcccgctattatcagtgctggtacaaagttagccc
aataggtggtcaggatggctattcaggctggcatcatcctcgtgattcgccgtgtcgcggtgcatatttcgcatcagttttgcaatgggattaagga
ggactaacccatgacaactaacaaccacccggcgcacggtcctgtatcactcgatcgcctgcaccagatacgcgaacacctgctgcatgatac
ccaatactcaaacggcgggaacagagcctacattctcgctgatgtattgaaggtgattgatggggctattgcccgcgagctggtacgccgtgag
catgcagcgtggtcacaggctactttcggcgatgtcggtccagttggtccgctgaagcacctttccaaagaagcgctcgaggctgctgctgaac
caggcgaccttagcgaatgggctgacatgcaattcctgttatgggatgcgcaacgtcgtgccggtatcagtgatgagcagattacccaggcaat
gataaaaaagctggctataaataaggttcgccaatggcctgagccgaaagacggggaacctcgattgcatatcaaagaacagtcagagcagg
agaaaaaataagaatgtttagcctgattcggcgcggtcaaatctacacggacagtagcaactggcccgtaattatccatagctgtagtgatcactc
ggtccgaattaaacgcaatgatggcgagctgagaacgattagcatcaaacgctttaacgaagattttgaacgagtggagcatgatgagtatcgc
aaaatatgtgccgaaatagagcaggaaacaaacctgaaaaacctacgtgcgatgcgtcgcggcaagattactgaatagccaaacaggagaat
atttaacgtgaacaacttaatgatcgaccttgagtccatgggcaaaaaaccgaatgcccctattgtctccattggtgccgtattcttcgatccgcaa
agcggtgaactgggtcaggagttttacaccgctgttaatcttgaaagcgctatggagcagggagcggtgccggatggtgacactattctgtggt
ggttaagacaaagctcagaagcacgatcagcaatctgtgttgatgatgcgatgccgatatcatctgccctatctgaactgagccatttcattaatcg
gcattctgataaccctaaatatttaaaagtttggggcaatggagctactttcgacaacgttatattgcgcggcgcatatgagcgtgccggccaggtt
tgcccgtggcaattttggaatgatcacgacgtcagaaccatcgtcacattaggcagatctgtaggtttcgatcctaagcgtgatatgccatttgatg
gggttgcacataacgcactggctgatgcccgccaccaggcgaaatatgtttcagcgatttggcagaaactaatcccaaccaccagcaacagct
aaagttttccccgggtgcagccgggataatggagaaataactatgagcaatattttccagttagctcccaacgattgggtttgtgaaagcgttttga
tcgcggttactgggctcaaacccggaaccatcctccgtgccagaaaagaatgctggatgattgggagggagtatatccacgtatcgcctgacg
gaaatcctaaaccttccagtgagtgcatgtataacagaaaggctgtagatgcctgggtcgcttcaatgaaaagcaagcaaccagggtgatttgat
gccatgaaaaaggtaagctcgtatcgctcttgggcgtctggaggtaacaccaatggataaagtcacatatccaacaggcgtcgaaaaccacgg
tggcacattacgcatctggtttaattttaaaggtaagcgtgtcagggaaagtctcggtgtccctgacaccgctaagaacaggaagatagccggg
gaactgcggacatcagtatgttttgccatccgcacaggaacctttgattatgcaacccagtttcctgactcccctaacctcaaggcttttggtgtaag
taaaaaagacattacagtgaaagaacttgaagaaaaatggctggatctgaaacggatggaaatctgcgcgaacgcatttaatcgctatgaatctg
tcgcaaggaatatggtgccgaggatcggaggtaatcgcctggtgtcagcagtaaccaaagaggaattgctgtatctgaggaaatatttgctaact
ggttatcagaatccgacgaaaaacaaagccccggcaaaagggcgaagcgttgttactgtgaactattacatgacgacaatggccggaatgtttc
agtttgctgcggatcacggttacttagaggtgaacccattcgagggaattaagcctctgaaaaaagccagggcagaaccagatcctctgtctcgt
gatgaatttattcgcctgatagatgcatgccggcatcagcagacgaaaaacctgtggtcattagcagtgtacacaggaatgcgtcacggggaac
tggtctccctggcctgggaagatatcgacctgaaggctggaacaattaccgtcagacgtaattatacgaaacttggtgagttcactctaccgaaa
accgaggcaagcacagatcgagtggtgcatcttatccagcccgcaatcagtatcctgaaaaatcaggctgaaatgacaaggctgggcaggca
atatcacattgaagtgcagttacgtgagtacggccgttcggtgaaccatgagtgtacattcgtctttaatccgcatgtggtcagacgcagtaagca
ggtcggatttatctaccgggtcgattcagtaggcgactcatgggaagcggcacttaagcgtgcggggatcagacacagaaaggcgtaccagt
cacgacacacctatgcgtgctggtcattatcagctggtgcaaaccctagttttattgccagtcagatggggcatgcgagcgcgcagatggtgttc
aatgtttacggtgcatggatggctgacagcagcgcagagcagatcgcaatgctgaatcagaagctggcagattttgccccattgatgccccata
gccacgagaacagtacgggaggattattaaaatcagtaagttaacccctaacgcccgtcatgttaactgtgtggagggtaacaccacgctttatg
ccctgccgaaacccgaggttgtcctgcgctggcgtgagcagaccacagatgacttccgcttctgttttaagtttccggcgaccatttcgcatcag
gcagcattacggcattgcgatgatttagtgactgaatttttgacccgcatgtcaccgttggctccgcgcattggacaatactggctgcaactgcctg
ccacattcggcccacgggagctgcctgcgctttggcattttctcgattctcttcccggtgaatttaattatggggtggaagtccgccatccacagttt
ttcgccaaaggggaagaggaacaaacgcttaatcgcggtttacatcagcgcggcgttaatcgggtgattttagacagccgcccggttcatgcag
cacgtccatacagtgaagctattcgcgacgctcaacgaaaaaaacctaaagttccggtacatgctgtactgacggcgaaaaatccactgatccg
ttttatcggtagtgatgatatgacgcaaaaccgggaattatttcaggtctggttacaaaaattagcgcagtggcatcagaccactacgccttatctttt
tttacatacgccagatattgcccaggccccggaactggtacataccctgtgggaagacttacgtaaaacgcttccagagatcggagcagttccg
gctattccacagcaatcttctcttttctgaatttgccacctatcatagacaggtgccatcggccattttaaagggagtttgtatggtaagcgcgctgta
tgccgttttaagtgcgttgttattaatgaagttctcttttgatgtcgttcgcctgcgaatgcagtaccgcgttgcctatggcgacggcggttttagcga
actgcaaagcgctattcgcattcatggtaacgcggtggaatatattcctatcgcgattgtgttgatgctgtttatggaaatgaatggcgcagaaacc
tggatggtgcatatttgcggcatcgttttgcttgctggtcgtctgatgcattattacggttttcatcaccgtctgttccgctggcgacgttctggcatga
gcgccacctggtgtgcgctgttgctgatggtgctggcgaatctttggtatatgccctgggagttggttttctccctgcgttagcgcacaatacgcca
ctttctttttcccggatttttacgttatgtctcaccgcgacacgctattttctgcccctatcgccagactgggcgactggacctttgatgaacgggtag
ctgaagtcttcccggatatgatccagcgttccgttcccggctattccaatattatttccatgattggtatgttagccgagcgcttcgttcaacctggta
cgcaggtttacgatctgggttgttctctgggcgcggcgacgctctcggtgcgtcgcaacattcatcatgataattgcaaaattattgccatcgacaa
ctccccggcgatgattgaacgctgccgtcgtcatattgacgcctataaagcccctacgccagtagacgttattgaaggtgatattcgcgatatcgc
cattgaaaacgcatcgatggtggtgctgaattttaccctgcaattcctggaaccttccgagcgccaggcgttactggataaaatttatcaagggct
gaacccgggcggtgcgctggtgctttcggaaaaattcagtttcgaagatgccaaagttggtgaactgctgttcaacatgcaccacgactttaaac
gtgccaacggttacagcgaactggagatcagccagaaacgcagcatgctggaaaacgtgatgctgaccgattccgtggaaacccataaagca
cgcctgcataaagccggttttgagcatagcgagctgtggttccagtgctttaactttggttcactggtggcattaaaagcagaggacgctgcatga
tcgactttggtaacttttattctctgattgccaaaaatcatctttcacactggctcgaaacgctgcccgcgcagattgctaactggcagcgcgagca
gcagcacgggctgtttaagcagtggtccaacgcggtggaatttctgcctgaaattaaaccgtatcgtctggatttattgcatagcgtaaccgccga
aagcgaagagccactgagcgccgggcaaattaagcgcattgaaacgctgatgcgcaacctgatgccgtggcgcaaagggccgttctcactgt
atggcgtcaacatcgataccgaatggcgttccgactggaaatgggatcgcgttatgccccatctttctgatttaaccgggcgcaccattcttgatgt
cggctgtggcagcggttatcacatgtggcgcatgattggcgcaggggcgcatctggcggtgggtatcgatcccacgcagctattcctctgcca
gtttgaagcagtgcgtaaactgctgggtaacgatcagcgcgcacatttgttaccgttaggtattgaacaacttccggcactgaaagcctttgatac
cgtcttttcgatgggcgtgctttatcatcgtcgttcaccgctggagcatctctggcagttaaaagaccaactggtgaatgaaggcgaactggtgct
ggaaacgctggttattgatggcgacgaaaacacggtgctggtgccgggcgatcgttacgctcaaatgcgtaatgtctatttcattccttccgcgct
ggcgctgaaaaactggctgaagaagtgtggttttgttgatattcgcattgcagatgtgagcgttaccaccacagaagagcagcgacgcaccgaa
tggatggtcaccgagtctctggccgattttctcgacccgcatgatccgggtaaaacggtggaaggttatcctgcgcctaaacgcgcggtgctgat
tgcgcgcaagccgtaaaggtctggtaatactgccggatgcggcgtgaacgccttatccggcctacaaagtcttgctaattcaatatattgcaggg
gctatgtaggcctgataagcatagcgcatcaggca

### Example 60. Testing of a Phage 3 Knockout of Wild Type E. coli Nissle (Prior to Removal of Chloramphenicol Cassette)

As described in Example 59, primers with 40 bp overhangs and homology to pKID3 had been used to create a knock out targeting a 10 kB region of the phage genome (lambda red recombineering followed by selecting for chloramphenicol resistance). Four clones obtained were selected. Using primers for to screen for the presence of an insertion of a chloramphenicol cassette in the phage 3 genome, the expected the 200 bp band was seen for all four clones indicating a positive insertion specific to the phage 3 genome.

The same 4 chloramphenicol resistant colonies were resuspended in 1 mL of LB in 14 mL culture tubes. Tubes were grown shaking at 37 C, 250 rpm. When cells reached early log phase, the cultures were split into two 500 ul aliquots; one aliquot was treated with mitomycin C (2 ug/mL); the other was left untreated. After 3.5 hrs, 400 ul of each culture was removed and 20 ul of chloroform was added. Samples were vortexed for 15 seconds and spun down in a centrifuge at max speed for 1 min. Table 96 shows the number of plaques counted. SYN-902 is WT Nissle comprising the pKD46 plasmid.

TABLE 96
Plaque Counts

| supernatant dilution | 0 | −1 | −2 |
|---|---|---|---|
| SYN-902 Φ3 knockout 1 no induction | 0 | | |
| SYN-902 Φ3 knockout 1 + MC | 0 | | |
| SYN-902 Φ3 knockout 2 no induction | 0 | | |
| SYN-902 Φ3 knockout 2 + MC | 0 | | |
| SYN-902 Φ3 knockout 3 no induction | 0 | | |
| SYN-902 Φ3 knockout 3 + MC | 0 | | |
| SYN-902 Φ3 knockout 4 no induction | 0 | | |
| SYN-902 Φ3 knockout 4 + MC | 0 | | |
| ATCC13706 (negative control) | 0 | | |
| SYN-PKU-710 + MC (positive control) | TMTC | not plated | TMTC |

TMTC: too many to count

In conclusion, no plaques were observed in any of the knock out strains, while the positive control produced a large number of plaques. These results indicate that deletion of a 10 kb internal region of phage 3 in wild type E. coli Nissle prevents the formation of plaques following mitomycin C treatment.

### Example 61. Phage Detection Assay in a Phage 3 Knockout Strain (Wild Type Nissle and SYN-PKU-1034)

This study was conducted to test whether A phage 3 knockout in wild type Nissle and in SYN-PKU-1034 results in a negative test in a plaque assay. Table 97 describes the strains used in this study.

TABLE 97
Strain Descriptions

| Strain Name | Description |
|---|---|
| SYN-PKU-1034 | Phenylalanine consuming strain which has a AIPS comprising the |
| | plasmid and genomic components shown in FIG. 61 of |
| | WO2017087580, the contents of which are herein incorporated by |
| | reference in their entirety. The plasmid comprising the toxin is |
| | medium copy, and in lieu of the bla gene, the plasmid contains pLac |
| | pFNR PAL and PheP (IPTG and low oxygen inducible PAL and |
| | PheP in tandem). The antitoxin is integrated into dapA gene, causing |
| | a dapA auxotrophy. The strain further contains LAAD integrated into |
| | the genomic Ara locus and also has a thyA auxotrophy |
| | (Para::LAAD; ΔthyA; dapA::antitoxin) |
| SYN-PKU- | SYN-PKU-1034 with phage knockout |
| 1034d″phi″3 | |
| SYN001 | Wild type E. coli Nissle |
| SYN-903 | SYN001 with phage knockout |

In this study, supernatants from E. coli Nissle (SYN01), SYN-903 (the phage 3 knockout in the Nissle background), SYN-PKU-1034d“phi”3 (phage 3 knockout in negative strain), SYN-PKU-710 (positive control), and ATCC13706 (negative control) were tested for the presence of phage.

PCR analysis using a primer set specific to a chloramphenicol (cm) cassette on one end and Phage 3 on the other had previously shown that the clone SYN-PKU-1034 phage 3 KO tested positive for the correct insertion of the chloramphenicol cassette (data not shown). Insertion of the CM cassette was performed as described above in Example 59.

For phage testing, strains (SYN01 (Nissle), ATC13706 (negative control), SYN-PKU-710, SYN-PKU-1033, SYN-PKU-1034, ATCC13706 (sensitive plaque indicator strain) were grown overnight in LB (with DAP 100 ug/mL where appropriate), 3 mL in a 14 mL culture tube shaking at 250 rpm at 37 C. Top agar composed of 7 g/L agar in LB media lacking yeast extract is prepared, melted, and kept at 45 C to maintain liquid.

Overnight cultures were used to inoculate 10 LB with DAP 100 ug/mL where appropriate in 125 mL baffled flasks at a 1:100 dilution. Each test strain was inoculated into 2 10 mL cultures: one flask with 2 ug/mL of mitomycin C to induce the phage and the second flask as a log phase uninduced control. All flasks were grown at 37 C shaking (250 rpm). All cultures were grown for 4.5 hours, diluted 10-fold in PBS in a 96-well plate, and plated for determination of cell counts. Spot dilutions (10 ul) spanning the 10\hat{ }−3 to the 10\hat{ }−8 dilutions were plated per plate (LB plates—DAP 100 ug/mL where appropriate), in duplicate, for each strain.

After completion of the cell counts, 1 mL from each culture (3 cultures per strain) was removed and placed in a 1.5 mL Eppendorf, 50 μL of chloroform was added, and tubes were vortexed for 15-30 seconds. Cells were spun down in a microcentrifuge for 2 minutes at maximum speed. Meanwhile, a 96-well plate was prepared to contain 180 μL of LB per well in columns 2-5 and 8-11. Dilutions of supernatant were performed in this plate. 200 μL of neat supernatant for each strain was added to column 1 for samples 1-8 and column 7 for samples 9-15. 10-fold dilutions were performed with a multi-channel pipette from columns 2-5 and 8-11.

To prepare sensitive control strain, 10 mL ATCC13706 was spun down at 4000×g in 15 mL falcon tube, supernatant was decanted and cells were resuspended in an equal volume of 10 mM magnesium sulfate. 14 mL culture tubes were set up and labelled for the appropriate strain and dilution of supernatant was added. To each tube, 100 ul of ATCC13706 cell suspension was added. Neat supernatant and its dilutions were added to the appropriate tubes and the cell/supernatant mixture was incubated for a minimum of 5 min.

After incubation, 3 mL of top agar was added to tubes and the mixture was immediately spread (by pouring) onto labelled LB plates. Plates were allowed to dry and then moved to a 37C static incubator, inverted, and incubated overnight. Results are shown in Table 987.

TABLE 98
Plaque Counts

| | Supernatant dilution | | | | | |
|---|---|---|---|---|---|---|
| | 0 | −1 | −2 | −3 | −4 | |
| | Final dilution (for calculating pfu/mL) | | | | | cfu/mL |
| | −1 | −2 | −3 | −4 | −5 | culture |
| ATCC13706 uninduced stationary | 0 | 0 | | | | 3.3 × 10\hat{ }9 |
| ATCC13706 + MC log | 0 | 0 | 0 | | | 1.8 × 10\hat{ }7 |
| SYN001 uninduced stationary | 20 | 3 | | | | 1.8 × 10\hat{ } |
| SYN001 + MC log | 249 | 18 | 2 | | | 0 |
| SYN-903 uninduced stationary | 0 | 0 | | | | 3.5e9 |
| SYN-903 + MC log | 0 | 0 | 0 | | | 1.5 × 10\hat{ }4 |
| SYN-PKU-1034dphi3 uninduced | 0 | 0 | | | | 1.8 × 10\hat{ }9 |
| stationary | | | | | | |
| SYN-PKU-1034dphi3 + MC log | 0 | 0 | 0 | | | 0 |
| SYN-PKU-710 uninduced stationary | TMTC | 65 | 2 | | | 2.1 × 10\hat{ }9 |
| SYN-PKU-710 + MC log | TMTC | TMTC | 289 | 72 | 3 | 0 |

TMTC = too many to count

As seen in the Table above, phage 3 is responsible for plaque formation, and a deletion in central genes within the phage chromosome can inhibit the formation of plaques. Gross observation did not suggest any sort of growth defect caused by deletion of the phage sequences.

### Example 62. Phage Testing of SYN-PKU-2001, SYN-PKU-1035, and SYN-PKU-1036

This study was conducted to assess the phage production in various phenylalanine strains in which the antibiotic cassette has been removed from the plasmid background, and to confirm that removal of this cassette does not allow for reformation of phage particles.

Additionally, a phage-3 knockout version of SYN-PKU-710, but still containing the cm cassette was tested. Table 99 describes the strains used in this study and related background strains.

TABLE 99
Strain Descriptions

| Name | Description |
|---|---|
| SYN-PKU- | malEK:: PfnrS-PAL3; malPT::PfnrS-PAL3; yicS/nepI:: PfnrS-PAL3; lacZ::PfnrS- |
| 710 | pheP, agaI/rsmI::PfnrS-pheP; Para::LAAD; exo/cea:: LacIPAL3; |
| | rhtC/rhtB::LacIPAL3; ΔdapA. |
| SYN-PKU- | SYN-PKU-710 with deltaphage3::cm |
| 2001 | |
| SYN- | Phenylalanine consuming strain which has a kill switch system comprising the |
| PKU1033 | plasmid and genomic components shown in FIG. 61 of WO2017087580, the |
| | contents of which are herein incorporated by reference in their entirety. The |
| | plasmid comprising the toxin is medium copy, and in lieu of the bla gene, the |
| | plasmid contains pLac pFNR PAL and PheP (IPTG and low oxygen inducible PAL |
| | and PheP in tandem). The antitoxin is integrated into dapA gene, causing a dapA |
| | auxotrophy. The strain further contains LAAD integrated into the genomic Ara |
| | locus and also has a thyA auxotrophy (Para::LAAD; ΔthyA; dapA::antitoxin); |
| | Note: SYN-PKU-1034has a weaker RBS upstream of the pheP portion of the PAL- |
| | pheP gene cassette in the AIP plasmid compared to SYN-PKU1033 |
| SYN-PKU- | SYN-PKU1033deltaphage 3 cured entirely |
| 1035 | |
| SYN-PKU- | Phenylalanine consuming strain which has a kill switch system comprising the |
| 1034 | plasmid and genomic components shown in FIG. 61 of WO2017087580, the |
| | contents of which are herein incorporated by reference in their entirety. The |
| | plasmid comprising the toxin is medium copy, and in lieu of the bla gene, the |
| | plasmid contains pLac pFNR PAL and PheP (IPTG and low oxygen inducible PAL |
| | and PheP in tandem). The antitoxin is integrated into dapA gene, causing a dapA |
| | auxotrophy. The strain further contains LAAD integrated into the genomic Ara |
| | locus and also has a thyA auxotrophy (Para::LAAD; ΔthyA; dapA::antitoxin); |
| | Note: SYN-PKU-1034has a weaker RBS upstream of the pheP portion of the PAL- |
| | pheP gene cassette in the AIP plasmid compared to SYN-PKU1033 |
| SYN-PKU- | SYN-PKU-1034deltaphage 3 cured entirely |
| 1036 | |

Cultures of ATCC13706 control, SYN-PKU-2001, SYN-PKU-1035, and SYN-PKU-1036 were grown overnight in 3 mL cultures in 14 mL tubes shaking at 37 C. Cells were then diluted into fresh media with mitomycin C (2 ug/mL). Plaque assays were performed essentially as described in Example 58. Plaque counts are shown in Table 100.

TABLE 100
Plaque Counts

| | Supernatant Dilution | | | | |
|---|---|---|---|---|---|
| | 0 | −1 | −2 | −3 | −4 |
| | Final Dilution (for calculating pfu/mL) | | | | |
| | −1 | −2 | −3 | −4 | −5 |
| ATCC13706 uninduced | 0 | 0 | N/T | N/T | N/T |
| stationary | | | | | |
| ATCC13706 + MC log | 0 | 0 | N/T | N/T | N/T |
| SYN-PKU-2001 uninduced | 0 | 0 | N/T | N/T | N/T |
| stationary | | | | | |
| SYN-PKU-2001 + MC log | 0 | 0 | N/T | N/T | N/T |
| SYN-PKU-1035 uninduced | 0 | 0 | N/T | N/T | N/T |
| stationary | | | | | |
| SYN-PKU-1035 + MC log | 0 | 0 | N/T | N/T | N/T |
| SYN-PKU-1036 uninduced | 0 | 0 | N/T | N/T | N/T |
| stationary | | | | | |
| SYN-PKU-1036 + MC log | 0 | 0 | N/T | N/T | N/T |
| SYN-PKU-710 uninduced | TMTC | 57 | 3 | 0 | N/T |
| stationary | | | | | |
| SYN-PKU-710 + MC log | TMTC | TMTC | 42 | 4 | 1 |

N/T—not tested;
MC—mitomycin C at 2 ug/mL

These results show that the knockout of phage 3 in SYN-PKU-710 resulted in no pfu from supernatant. Curing cassette from phage 3 knockout in plasmid strains also led to no pfu formation in supernatant.

### Example 63. Generation and Analysis of SYN-PKU-2002

A version of the phenylalanine consuming strain SYN-PKU-710 in which phage 3 has been knocked out and the chloramphenicol cassette has been cured was analyzed. First, plaque formation was assessed in a plaque assay. Next, the ability of the strain to produce transcinnamic acid (TCA) was compared with SYN-PKU-710 to determine whether removal of the phage would cause any changes in phenylalanine consumption activity in vitro. Sanger sequencing of the relevant Phe degrading regions was performed to confirm that phenylalanine consuming circuitry.

First, to cure the chloramphenicol cassette, SYN-PKU-2001 (SYN-PKU-7103 with a delta phage 3::cm insertion) was transformed with pCP20, and 4 carbenicillin resistant transformants were selected for analysis. Removal of the chloramphenicol cassette was first confirmed by PCR. These transformants were grown overnight at 42 degrees to cure the pCP20 plasmid and were then streaked on LB plates (plain LB plates as control to ensure no growth, LB plus carbenicillin to confirm loss of plasmid and LB chloramphenicol to confirm removal of the chromosomal Cm cassette respectively, and LB Dap, on which the strains grow (dap auxotrophy)). Cells were also streaked on Phe agar and spotted ferric chloride to ensure LAAD activity was still present. All 4 clones turned dark green immediately, signifying a positive LAAD result. One clone was maintained for further analysis and named SYN-PKU-2002.

Next SYN-PKU-2002 was prepared for phage testing using ATCC13706 as a negative control and SYN-PKU-710 as a positive control. Plaque assay was carried out essentially as described in Example 58. Phage testing results showed no plaques in uninduced and mitomycin C induced supernatants from SYN-PKU-2002 (data not shown).

Next, flasks were prepared for testing activity of SYN-PKU-2002. Activity was tested with or without IPTG induction (aerobically), and with anaerobic induction. To perform this assay, an overnight culture of SYN-PKU-2002 was back-diluted 1:100 in 10 mL cultures in 125 mL baffled flasks (3 flasks inoculated, 1 for each condition). Cells were allowed to grow for 1 hr and 40 minutes, at which point they had entered early log phase. One flask remained untouched, the other received IPTG at 1 mM final concentration, and third flask was moved to the anaerobic chamber supplying 90% N₂, 5% CO₂, and 5% H₂. The cells were allowed to induce for 4.5 hours. The TCA production assay was then performed according to the standard protocol as described elsewhere herein. Essentially, bacteria were resuspended in assay buffer containing 50 mM phenylalanine. Aliquots were removed from cell assays every 20 min for 1.5 hrs for trans-cinnamate quantification by absorbance at 290 nm.

As seen in Table 101, both IPTG and anaerobic induction of SYN-PKU-2002 was observed.

TABLE 101
In vitro Activity Measurements

| | | | | rate |
|---|---|---|---|---|
| | CFU/ | | | (umol/hr/ |
| Strain | ASSAY | 30 | 60 | 1e9cells) |
| SYN-PKU-2002 − IPTG A | 8.16E+07 | 0.00488 | 0.015122 | 0.19 |
| SYN-PKU-2002 − IPTG B | 8.57E+07 | 0.00267 | 0.009158 | 0.11 |
| SYN-PKU-2002 + IPTG A | 8.00E+07 | 0.1345 | 0.273565 | 3.42 |
| SYN-PKU-2002 + IPTG B | 8.93E+07 | 0.13411 | 0.263227 | 2.95 |
| SYN-PKU-2002 − O2 A | 7.25E+07 | 0.09474 | 0.170983 | 2.36 |
| SYN-PKU-2002 − O2 B | 8.62E+07 | 0.08878 | 0.176152 | 2.04 |

All samples were sent for Sanger sequencing and confirmed that all of the Phe-degrading relevant regions in SYN-PKU-2002 were sequence accurate.

In conclusion, SYN-PKU-2002 does not produce plaque forming units (pfus) against ATCC13706, when supernatant was used form cultures that were uninduced or induced with mitomycin C. SYN-PKU-2002 contains no antibiotic markers and no plasmids. It also has phenylalanine degrading activity both with IPTG and anaerobic induction. All of the relevant regions involved in phenylalanine degradation were sequenced. Therefore, SYN-PKU-2002 appears functionally equivalent to SYN2619 except that it does not produce phage.

### Example 64. SYN-PKU-2002 In Vitro Activity Measurements

A 1:100 back-dilution from overnight culture of SYN-PKU-2002 was grown to early log phase for 1.5 h before moving to the anaerobic chamber for 4 hours in the presence of 1 mM IPTG and 0.1% arabinose for induction as described herein. To perform activity assay, 1e8 cells were resuspended and incubated in assay buffer (M9 media with 0.5% glucose, 50 mM Phe, and 50 mM MOPS with 50 mM phenylalanine). Supernatant samples were taken over time and TCA (the product of PAL) was measured by absorbance at 290 nm to determine the rate of TCA production/PAL activity. Phenylpyruvate was measured using LCMS methods described herein. Results are shown in FIG. 16A and FIG. 16B.

### Example 65. Analysis of Activity of SYN-PKU-2002 In Vivo—the Effect of Phage 3 Deletion from SYN-PKU-710 on In Vivo Activity in the Enterorecirculaton Model of Phenylketonuria (PKU)

The activity of SYN-PKU-2002 to produce hippurate in vivo is assessed. In this study, SYN-PKU-710 and SYN-PKU-2002 are grown in flasks and induced anaerobically, and the effect of the phage deletion on the ability of the SYN-PKU-2002 to produce hippurate and reduce serum Phe is assessed through comparison with the isogenic phage containing strain SYN-PKU-710 in vivo.

SYN-PKU-710 and SYN-PKU-2002 overnight cultures are each used to inoculate 4 2 L flasks containing 500 mL of LB with DAP 100 ug/mL. These cultures are grown for 1 hr and 45 min and then moved to the anaerobic chamber supplying 90% N₂, 5% CO₂, and 5% H₂ for 4 hours. Cells are then spun down at 4600×G for 12 min and resuspended in 10 mL of formulation buffer (Glycerol: 15% (v/v), Sucrose: 10% (w/v) (100 g/L), MOPS: 10 mM (2.1 g/L), NaCl: 25 mM (1.46 g/L)). Several 40 ul aliquots are removed to be used for cell counting and activity determination. Activity is determined essentially as described in Example 63 and is measured for SYN-PKU-710 and SYN-PKU-2002, respectively. The viability as determined by cellometer count for SYN-PKU-710 and SYN-PKU-2002. SYN-PKU901 (streptomycin resistant Nissle, 8e10 cfu/ml) is used as a control. Cells are brought up to 10 ml with PBS, and then mixed 9:1 with 1 M bicarbonate to achieve a final concentration of 100 mM bicarbonate, in preparation for daily gavage of 4e10 cells (3×300 ul doses).

To compare the efficacy of phage positive SYN-PKU-710 vs isogenic phageless strain SYN-PKU-2002 in vivo, strains are administered to in enu2 mice. SYN-PKU901 is administered as a control. Female BTBR-Pah enu2 mice (20-35 g) from CRL (GEMS) within 6-18 wks of age are allowed to acclimate to facility for at least 2 days. Animals are placed on phenylalanine deficient diet (Teklad TD.97152) and are given a phenylalanine enriched water at 0.5 g/L of L-Phenylalanine (Sigma) and 5% sucrose/L (Sigma) at least 2 days prior to starting study. Phenylalanine enriched water is removed for duration of study and is replaced with normal water.

On the day of the study, mice are randomized into treatment groups according to weight as follows: Group 1: H2O (n=9); Group 2: SYN-PKU901 (n=9); Group 3: SYN-PKU-710 (n=9); Group 4: SYN-PKU-2002 (n=9). Blood samples are collected by sub-mandibular skin puncture to determine baseline phenylalanine levels. Mice are then administered single dose of phenylalanine by subcutaneous injection at 0.1 mg per gram body weight, according to the average group weight. At 1, 2 and 3 h post Phe challenge, the bacteria (or water) are administered to mice by oral gavage (3×300 ul). Animals are bled and urine is collected from all animals up to 4 h post Phe challenge. Blood samples are kept on ice until processing for plasma in a centrifuge (2000 g for 10 min at 4 C) within 20 min of collection. Plasma is then transferred into a 96-well plate for MS analysis. Urine is collected in 5 mL tubes and volumes are recorded before transferring samples to MS for hippurate analysis.

### Example 66. In Vivo Serum Phe and Hippurate Measurements Upon Administration of SYN-PKU-2002

The activity of SYN-PKU-2002 was assessed in vivo. To prepare the cells for the study, SYN-PKU901 and SYN-PKU-2002 overnight cultures were each used to inoculate 4 2 L flasks containing 500 mL of LB with DAP 100 ug/mL. These cultures were grown for 1 hr and 45 min and then moved to the anaerobic chamber supplying 90% N₂, 5% CO₂, and 5% H₂ for 4 hours. Cells were then spun down at 4600×G for 12 min and resuspended in 10 mL of formulation buffer (Glycerol: 15% (v/v), Sucrose: 10% (w/v) (100 g/L), MOPS: 10 mM (2.1 g/L), NaCl: 25 mM (1.46 g/L)). Several 40 ul aliquots were removed to be used for cell counting and activity determination. The viability as determined by cellometer count (in quadruplicate) 6.94e10 cfu/ml (+/−5.78e9).

Activity was determined using a plate based assay. Briefly, 1×10⁸ cfu as determined by cellometer were added to 1 ml of prewarmed assay buffer (1×M9 minimal media containing 0.5% glucose, 50 mM MOPS, and 50 mM phenylalanine) in a microfuge tube, vortexed briefly, and immediately placed in a heat block or water bath at 37 degrees Celsius for static incubation (t=0). Supernatant samples from cells re-suspended in assay buffer were analyzed for the abundance of TCA over several time points using spectrophotometer at an absorbance of 290 nm. The accurate OD₂₉₀ window for TCA detection occurs in a relatively narrow concentration range. For this reason, supernatant samples were diluted to ensure that the absorbance measurement fell into the linear range for detection. Measurements were compared to a TCA standard curve. Activity was determined to be 2.72 umol/hr/1e9 cfu (+/−0.15 umol/hr/1e9 cfu).

Beginning 4 days prior to the study (i.e., Days −4-1), Pah ENU2/2 mice (˜11-15 weeks of age) were maintained on phenylalanine-free chow and water that was supplemented with 0.5 grams/L phenylalanine. On the day of the study, mice were randomized into treatment groups according to weight as follows: Group 1: SYN-PKU901 (n=9); Group 2: Group 2: SYN-PKU-2002 (n=9). Blood samples were collected by sub-mandibular skin puncture to determine baseline phenylalanine levels. Mice were then administered single dose of phenylalanine by subcutaneous injection at 0.1 mg per gram body weight, according to the average group weight. At 1, 2 and 3 h post Phe challenge, the bacteria (or water) were administered to mice by oral gavage (3×250 ul). Whole blood was collected via submandibular bleed at each time point. Urine collection in metabolic caging commenced immediately after the 1^{st} bacterial dose and continued to be collected for the duration of the study (4 hours).

Blood samples were kept on ice until processing for plasma in a centrifuge (2000 g for 10 min at 4 C) within 20 min of collection. Plasma was then transferred into a 96-well plate for MS analysis. Urine was collected in 5 mL tubes and volumes were recorded before transferring samples to MS for analysis. Results are shown in FIG. 17A and FIG. 17B and show that SYN-PKU-2002 causes decreased changes in phenylalanine post-Phe injection and produces hippurate, in a similar manner as SYN-PKU-710.

### Example 67. Efficacy of SYN-PKU-2002 in the ENU2 Murine Model of Phenylketonuria

The objective of this study was to examine the dose dependent in vivo activity of the phenylalanine (Phe)-degrading probiotic SYN-PKU-2002 in the ENU2 murine model of phenylketonuria (PKU), as measured by the generation of urinary hippurate (HA), trans-cinnamate (TCA), and phenylalanine (Phe) following oral SYN-PKU-2002 administration. Efficacy of SYN-PKU-2002 as it relates to PKU was measured by the ability of orally administered SYN-PKU-2002 to decrease plasma Phe levels in Pah^{enu2/enu2} mice independent of dietary Phe intake, in conjunction with the appearance of plasma TCA and HA, and was also assessed by measuring these metabolites in the plasma following Phe administration by subcutaneous (SQ) injection.

In the initial study, female Pah^{enu2/enu2} mice maintained on Phe-deficient diet were weighed and then randomized by weight into 2 treatment groups (n=9 each). Blood (lithium heparin used as anticoagulant) was obtained from each mouse via submandibular bleed (T=0 h). Mice then received an SQ injection of Phe (0.1 mg/g) and were immediately placed in metabolic cages (3 mice/cage) for collection of urine. At 1, 2, and 3 h post Phe injection, mice were gavaged orally with either control SYN-PKU901 (Group 1) or SYN-PKU-2002 (Group 2) cells that had been grown and pre-induced in shake flasks (5×10¹⁰ cells total dose, evenly split across the 3 hourly gavages). At 4 h post-injection, plasma (lithium heparin used as anticoagulant) was obtained by submandibular bleed and the urine was collected. Liquid chromatography-tandem mass spectroscopy (LC-MS/MS) was used to measure concentrations of Phe, TCA, and HA in plasma and urine.

The study was performed similarly to the study described in Example 65 with the exception that the SYN-PKU-2002 test article used to gavage mice was grown and activated in a bioreactor using a process similar to the one intended for scale up of drug substance. Female ENU2 mice (n=63) maintained on Phe deficient diet were weighed and then randomized by weight into 7 treatment groups (n=9 each). Mice were then given a SQ injection with Phe (0.1 mg/g) and immediately placed in metabolic cages for urine collection. SYN-PKU-2002 was gavaged orally at 1, 2, and 3 h post Phe injection to 6 dose groups (n=9/dose group split into 3 metabolic cages of 3 mice/cage). Dose groups received 1×10¹¹, 5×10¹⁰, 2.5×10¹⁰, 1.25×10¹⁰, 6.25×10⁹, or 3.13×10⁹ cells in total, equally split across the 3 hourly gavages. SYN-PKU901 was gavaged to a control group (n=9) at the highest dose of 1×10¹¹ cells. Urine was collected over 4 h. Plasma (lithium heparin used as anticoagulant). was obtained by submandibular bleed at T=0 h and at T=4 h at the highest dose group (1×10¹¹ cells) for both SYN-PKU-2002 and SYN-PKU901-treated mice for the determination of plasma Phe changes. LC-MS/MS was used to measure plasma and urinary Phe, HA, and TCA. A quantitative LC-MS/MS method was not available to measure phenylpyruvate in plasma or urine.

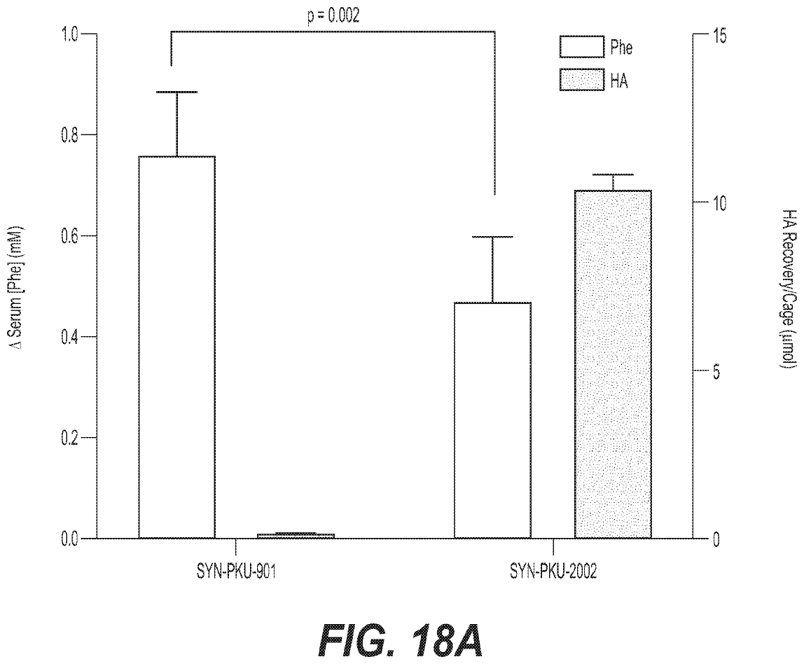

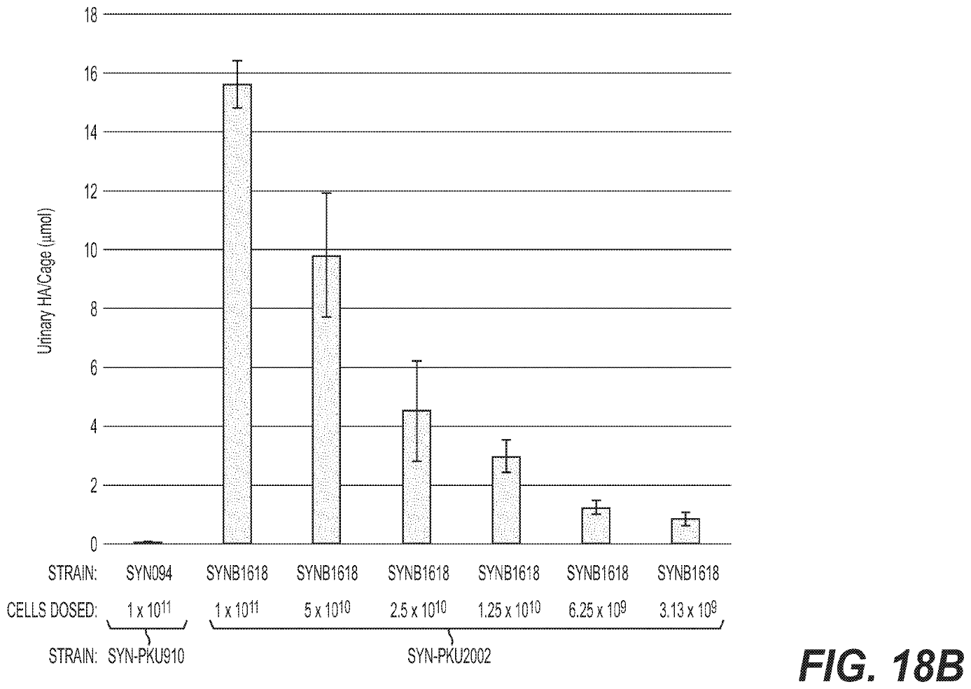

Results are shown in FIG. 17A and FIG. 18B. In the first study, following SQ Phe injection (0.1 mg/g), a plasma Phe increase was significantly blunted in ENU2 mice that received an oral dose of 5×10¹⁰ SYN-PKU-2002 cells compared to an equal dose of the control strain SYN-PKU901 (37.7% decrease, p=0.0002). The quantity of urinary HA excreted during the 0-4 h sampling period of the study was determined by multiplying the urinary HA concentration by the volume of urine collected. Urinary HA concentrations were below the lower limit of quantitation (BLLOQ; <0.04 mM) in mice treated with the control strain SYN-PKU901, while concentrations in mice administered SYN-PKU-2002 were high (ranging from 6.59-7.97 mM), resulting in measurable HA excretion over 4 hours (10.24±0.59 μmol). TCA and HA were not observed in the plasma of any of the mice at T=0 but were detected 4 h post administration in the group receiving SYN-PKU-2002. TCA and HA were not measurable in the plasma of mice treated with the control strain SYN-PKU901 at any time point, indicating that TCA increases were due to SYN-PKU-2002 activity and demonstrating SYN-PKU-2002 function in vivo. Urinary concentrations of Phe and TCA were BLLOQ in all samples.

In the second study, efficacy was also observed when SYN-PKU-2002 was grown in a bioreactor under conditions similar to the process used for scale-up of drug substance. Following SQ Phe injection (0.1 mg/g) in ENU2 mice, a plasma Phe increase was significantly blunted in the group that received an oral dose of 1×10¹¹ SYN-PKU-2002 cells compared to an equal dose of SYN-PKU901 cells (29.3% decrease, p=0.02). Additionally, increases in urinary HA excretion were observed in mice treated with SYN-PKU-2002 in a dose-dependent manner (15.61±0.81, 9.81±2.11, 4.52±1.70, 2.97±0.55, 1.24±0.23, and 0.86±0.25 μmol of HA were excreted in mice dosed with 1×10¹¹, 5×10¹⁰, 2.5×10¹⁰, 1.25×10¹⁰, 6.25×10⁹, or 3.13×10⁹ SYN-PKU-2002 cells, respectively). Mice treated with 1×10¹¹ SYN-PKU901 cells, the highest dose used for SYN-PKU-2002, did not excrete a large amount of HA (0.08±0.02 μmol). Similar to the first study, TCA and HA were not observed in the plasma of any of the mice at T=0, but were detected at 4 h post-administration in the group receiving SYN-PKU-2002 and not in animals receiving SYN-PKU901. HA excretion in urine correlated well with the amount of SYN-PKU-2002 cells dosed, indicating that the metabolite HA is a promising biomarker of in vivo SYN-PKU-2002 activity.

In conclusion, these data demonstrate the Phe-metabolizing activity of SYN-PKU-2002 in vivo by increasing the circulation of plasma TCA and HA and greatly increasing the amount SYN-PKU-2002 activity in the ENU2 mouse model. The second part of the study showed that SYN-PKU-2002 grown in a bioreactor using a process intended for the scale-up of drug substance was active in vivo. In this experiment, plasma Phe levels increased in mice when given an SQ injection of Phe along with orally administered control strain SYN-PKU901; however, mice orally dosed with SYN-PKU-2002 had a significantly blunted spike in plasma Phe concentrations following SQ Phe injection. Importantly, this result indicates that systemically circulating Phe reaches the intestine through enterorecirculation and is subsequently broken down by orally administered SYN-PKU-2002 in the gastrointestinal (GI) tract. This experiment demonstrated that SYN-PKU-2002 can decrease circulating Phe levels in blood, independent of the dietary intake of protein

### Example 68. Viability Comparison Between Phage Containing and Phage Free Phenylalanine Consuming Strains In Vivo

To identify potential differences in viability, transit or colonization between the phage containing phenylalanine strain SYN-PKU-710 and its phage-free counterpart SYN-PKU-2002, an in vivo competition study was conducted and a competitive index of SYN-PKU-710 vs the isogenic phageless SYN-PKU-2002 strain was generated. Because neither SYN-PKU-710 or SYN-PKU-2002 have antibiotic cassettes, marked strains that are uncured of antibiotic resistance were used in this study. For SYN-PKU-710, the chloramphenicol/kanamycin (cm/kan) marked strain SYN-PKU-713 was used. For SYN-PKU-2002, the chloramphenicol (cm) marked strain SYN-PKU-2001 was used. Table 102 lists strains relevant to this study.

TABLE 102
Strain Descriptions

| Strain Name | Description |
|---|---|
| SYN-PKU- | malEK:: PfnrS-PAL3; malPT::PfnrS-PAL3; yicS/nepI:: PfnrS-PAL3; |
| 710 | lacZ::PfnrS-pheP, agaI/rsmI::PfnrS-pheP; Para::LAAD; exo/cea:: |
| | LacIPAL3; rhtC/rhtB::LacIPAL3; ΔdapA. |
| SYN-PKU- | malEK:: PfnrS-PAL3; malPT::PfnrS-PAL3; yicS/nepI:: PfnrS-PAL3; |
| 713 | lacZ::PfnrS-pheP, agaI/rsmI::PfnrS-pheP; Para::LAAD; exo/cea:: |
| | LacIPAL3:cm; rhtC/rhtB::LacIPAL3:kn; ΔdapA (chloramphenicol and |
| | kanamycin resistance) |
| SYN-PKU- | malEK:: PfnrS-PAL3; malPT::PfnrS-PAL3; yicS/nepI:: PfnrS-PAL3; |
| 2002 | lacZ::PfnrS-pheP, agaI/rsmI::PfnrS-pheP; Para::LAAD; exo/cea:: |
| | LacIPAL3; rhtC/rhtB::LacIPAL3; ΔdapA; with phage3 KO, cured of cm |
| | resistance |
| SYN-PKU- | malEK:: PfnrS-PAL3; malPT::PfnrS-PAL3; yicS/nepI:: PfnrS-PAL3; |
| 2001 | lacZ::PfnrS-pheP, agaI/rsmI::PfnrS-pheP; Para::LAAD; exo/cea:: |
| | LacIPAL3; rhtC/rhtB::LacIPAL3; ΔdapA; with insertion of phage3 KO |
| | frag::cm and cured of pKD46 (not cured of chloramphenicol resistance; |
| | with chloraphenico resistance |

Briefly, overnight cultures of SYN-PKU-713 and SYN-PKU-2001 were used to inoculate 100 mL of LB (also containing DAP 100 ug/mL and appropriate antibiotics) in 500 mL baffled flasks. The cultures were grown for 6 h, at which point they were spun down in a centrifuge at 4000×g for 15 minutes for collection. Supernatant was discarded. Cells were resuspended in approximately 7 mL of formulation buffer (Glycerol: 15% (v/v), Sucrose: 10% (w/v) (100 g/L), MOPS: 10 mM (2.1 g/L), NaCl: 25 mM (1.46 g/L)). Cells were aliquoted into 650 μL aliquots and frozen at −80 C. Smaller aliquots were removed for cell plating and viability determination. Using a cellometer, viability was shown to be very high, and cell numbers fairly comparable between the strains.

All cell counts for this study (including input) were obtained by plating so that input (gavage) and output (feces) could be consistently compared. The two 650 μL aliquots were thawed on ice, mixed 1:1, diluted in sodium bicarbonate (9 parts cells:1 part 1M bicarbonate (144 uL), and then plated out in quadruplicate for quantitation on kan/cm and cm (from −8->−10 dilution by 10-fold 10 uL spot plating).

On Day 1, wild type B6 mice (n=6) were gavaged with 200 ul cells (approximately 3×10\hat{ }9 of each strain). Fecal pellets were collected 6 hours prior and 6 hours post gavage. On days 2 and 3, mice were gavaged with 200 ul cells and fecal pellets were collected 6 hours prior and 6 hours post gavage. On days 4 and 5, fecal pellets were collected.

Each day, collected fecal pellets were weighed in tubes containing 1 ml PBS and homogenized. To determine the CFU of Nissle in the fecal pellet, the homogenized fecal pellet was serially diluted, and each sample was plated onto LB plates containing chloramphenicol and plates containing kanamycin. The plates were incubated at 37° C. overnight, and colonies were counted. To determine the amount of the two strains in the feces, total recombinant Nissle was counted on cm plates, and the number of SYN-PKU-713 obtained on kan plates was subtracted from the total amount to determine the number of SYN-PKU-2001 CFUs.

Results are shown in FIG. 19 and the competitive index (SYN-PKU-713 input/SYN-PKU-2001 output are shown in Table 103.

TABLE 103
Competitive Index of Output
Competitive index of output (SYN-PKU-713
output/SYN-PKU-2001 output):

| Time (h) | M1 | M2 | M3 | M4 | M5 | M6 | AVG |
|---|---|---|---|---|---|---|---|
| 6 | 0.6 | 1.1 | 2.1 | 1.6 | 1.8 | 1.4 | 1.4 |
| 24 | 1.7 | 1.2 | 1.2 | 22.5 | 2.9 | 1 | 5.1 |
| 30 | 1.2 | 1 | 0.9 | 1.5 | 1.8 | 0.9 | 1.2 |
| 48 | 1.5 | 1.1 | 1 | 1.1 | 1.5 | 0.1 | 1.1 |
| 54 | 1 | 1.2 | 0.9 | 1.1 | 1.2 | 1.8 | 1.2 |
| 74 | 0.4 | 0.4 | 0.8 | 1.8 | 1.6 | 0.7 | 0.9 |
| 100 | NA | NA | NA | NA | NA | NA | NA |

Results indicate that there is no large difference in transit or colonization between the phage-free PKU strain of Nissle SYN-PKU-713 and SYN-PKU-2001.

### Example 69. General Bacteriophage Testing Protocol for Strains Derived from E. coli Nissle

The following procedure detects a DNA sequence found in EcN prophage/phage. It amplifies phage and prophage DNA within the EcN genome in SYN-PKU-2002 as the primers amplify outside of the region of prophage deleted to generate the bacterial strain for SYN-PKU-2002.

The validated bacteriophage (phage) method, GP-V708, Plaque Assay of Bacterial Virus from E. coli Using Mitomycin C Induction, measures the presence of phage using a plaque assay. Testing confirms that phage detected is from the endogenous EcN prophage and not from a contaminating phage, or adventitious agent.

The plaque assay starts with a loopful or scrape of frozen sample, negative and positive bacterial cultures which are grown in enriched medium supplemented with 10 mM thymidine at 37±2° C. and shaken at 200-300 RPM overnight. A portion of the sample, positive control (E. coli, EMG 2: K [lambda], ATCC 23716, or equivalent) and negative control (E. coli, ATCC 13706, or equivalent) is removed and centrifuged, and each supernatant examined in a plaque assay for the presence of bacteriophage. Mitomycin C, at a final concentration of 2 μg/mL, is added to the remaining sample, positive and negative bacterial cultures. The cultures are then placed at 37±2° C. and shaken at 300-400 RPM until lysis occurs in the positive control (−4.5 hours). Each culture is treated with chloroform, centrifuged, and a 0.1 mL aliquot of the supernatant is examined for the presence of bacteriophage. To accomplish this, supernatants are mixed with phage-sensitive E. coli strain ATCC 13706, mixed with 0.7% agarose solution, and plated as a lawn atop LB agar. The test is considered valid if plaques are present in the positive control and no plaques are present in the negative control.

Plates containing the strain of interest that contain enumerable plaques are identity tested by end point PCR to confirm that plaques are from phage generated from the endogenous EcN prophage. This is accomplished by first creating an agar plug of individual plaques with a 2 μL pipet tip and resuspending each plug in 0.5 mL of deionized water. Plug resuspensions are vortexed for 15 sec and 1 μL is used as template for PCR. As a negative control, an agar plug from the same plates is created from an area which does not contain plaques. A total of 10 plaques are tested per sample, when available. In cases when less than 10 plaques are formed per sample, all plaques are analyzed. One (1) negative control plug is tested per sample. As additional controls, pure broth cultures of EcN and ATCC 13706 are grown overnight in enriched medium at 37±2° C. with shaking at 200-300 RPM. One hundred (100) μL of stationary cultures are added to 0.2 mL thin wall tubes and heated at 98° C. in an Eppendorf Mastercycler Pro thermocycler for 10 min to prepare genomic lysate to be used as template for PCR.

Overall samples and controls used as template for each PCR assay were:
a. Sample: 1 μL of up to 10 resuspended plaques picked from an enumerable plate
b. Negative control: 1 μL of 1 plug picked from a non-plaque area from the enumerable sample plate
c. Negative control: 1 μL from lysed stationary culture of E. coli, ATCC 13706, or equivalent strain (plaque indicator strain)
d. Positive control: 1 μL from lysed stationary culture of the strain of interest cells

PCR is used to amplify a region of EcN-specific phage followed by detection of PCR amplified fragment at the endpoint of the reaction via gel electrophoresis. Primers (Integrated DNA Technologies, Skokie, IL) used for the amplification of EcN-specific phage are shown in Table 104. The primers were selected after careful examination of the EcN phage and amplify a region within the EcN-specific phage but do not bind to any region in the genomic DNA within the phage-sensitive E. coli strain ATCC 13706 that is used for plaque detection.

TABLE 104
EcN-Phage-specific PCR Primers

| Primer | | SEQ ID |
|---|---|---|
| description | Primer sequence | NO |
| EcN phage | 5-gcatcaatcagtgattggc-3 | SEQ ID |
| forward primer | | NO: 132 |
| EcN phage | 5-ACGTCTGAATATACGGGCTG-3 | SEQ ID |
| reverse primer | | NO: 133 |

Abbreviations: EcN = E. coli Nissle 1917

The reactions are carried out in 0.2 mL thin wall tubes using an Eppendorf Mastercycler Pro PCR machine. A master mix is prepared for the phage-specific primers. Primers are reconstituted to a final concentration of 100 μM in nuclease-free sterile water. Master mixes are prepared per the recipe in Table 105.

TABLE 105
PCR Master Mix Preparation

| Reagent | Per rxn (μL) | Final Concentration | |
|---|---|---|---|
| Nuclease-free sterile water | 8.8 | N/A | |
| Forward primer (10 μM) | 0.1 | 0.12 | μM |
| Reverse primer (10 μM) | 0.1 | 0.12 | μM |
| 2X MyTaq ™ Red Mix | 10 | 0.2 | U/μL |

Abbreviations:
N/A = not applicable;
PCR = polymerase chain reaction;
rxn = reaction

Nineteen (19) L of PCR master mix is added to each 0.2 mL PCR tube followed by addition of 1 μL of PCR template into individual tubes. The tubes are capped, placed in the PCR machine, and the reaction cycle run following parameters in Table 106.

TABLE 106
PCR Reaction Cycle

| Stage | Temperature (° C.) | Time (min:sec) |
|---|---|---|
| 1 | 95 | 5:00 |
| 2 | 95 | 0:15 |
| | 58 | 0:15 |
| | 72 | 0:10 |
| 3 | Repeat Stage 2: | N/A |
| | 25 cycles | |
| 4 | 72 | 1:00 |
| 5 | 45 | N/A |

Abbreviations:
° C. = degrees Celsius;
min = minute(s);
PCR = polymerase chain reaction;
sec = second(s)

Five μL of each standard, sample, or control reaction are loaded onto a 0.8% agarose gel for separation by electrophoresis and visualization using a Syngene UV transilluminator. The approximate size of the amplified band is 350 bp for the presence of EcN-specific phage while the negative control does not produce any bands.

### Example 70. SYN-PKU-2002 Toxicology Study

The dosing duration for the GLP toxicology study is 28 days in male and female CD-1 mice with BID dosing (twice daily). Doses cover an approximate range of 1×109, 1×1010, and 1×1011 CFUs. The highest concentration is the maximum feasible dose. Mice are assessed for test article related mortality, clinical observations, body weight, hematology, clinical chemistry, and macroscopic and microscopic pathology. Following cessation of dosing, fecal samples are taken from mice for several days to assay for the presence of SYN-PKU-2002 DNA using qPCR analysis to determine the decrease in SYN-PKU-2002 over time. Blood also is evaluated for the presence of SYN-PKU-2002 DNA using qPCR. The design of the 28-Day Mouse GLP Toxicology Study is outlined in Table 107.

TABLE 107
Design for 28-Day Mouse GLP Toxicology Study
4-Week GLP Toxicity Study of SYN-PKU-2002 in Mice with 2-Week Recovery Phase

| Purpose | Determine subchronic toxicity of test compound | | | |
|---|---|---|---|---|
| | Determine reversibility of test compound effects | | | |
| | Determine TK profile and exposures associated with | | | |
| | findings | | | |
| | Determine excretion/clearance of SYN-PKU-2002 in feces | | | |
| GLP Status | GLP | | | |
| Species, Strain | Mouse, CD1 | | | |
| Test Article | SYN-PKU-2002 | | | |
| Route/Number | Oral (PO)/twice daily (BID, 6-8 hours between the doses) | | | |
| Doses | | | | |
| Treatment Duration | 28 days | | | |
| Recovery | 14 days | | | |
| | | Dose/day | Numbers of Animals | |
| Study Design | Group | (CFU)* | M | F |
| | 1 (Control) | 0 | 12 MS + 6 Rec | 12 MS + 6 Rec |
| | 2 | ~2 × 10⁹ CFU | 12 MS + 6 Rec | 12 MS + 6 Rec |
| | 3 | ~2 × 10¹⁰ CFU | 12 MS | 12 MS |
| | 4 | ~2 × 10¹¹ CFU (maximum | 12 MS | 12 MS |
| | | feasible dose) | | |

| | |
|---|---|
| | MS = main study; Rec = recovery |
| | *draft doses estimated based on prior programs |
| | CFU = colony forming units |
| Mortality | All animals, 2X daily (AM, PM) |
| Clinical Observation | MS animals, 1X daily, detailed observations |
| Body Weight | All animals - pretest, weekly, and prior to necropsy |
| | (fasted) |
| Food Consumption | MS animals, weekly |
| Veterinary Exams | MS animals, standard assessments |
| Ophthalmology | All animals - pretest and prior to necropsy (Main study |
| | animals. Recovery animals if significant ophthalmic changes are |
| | noted at end-of-dosing. |
| Hematology/Serum | MS animals - at least 3/sex/group for hematology |
| Chemistry | MS animals - at least 3/sex/group for clinical chemistry |
| | Recovery - at least 3/sex/group for hematology |
| | Recovery - at least 3/sex/group for clinical chemistry |
| Urinalysis | Main study and recovery animals (Week 4 and Week 6) |
| Necropsy | Day 28 (Main study animals) and Day 42 (Recovery |
| | animals) |
| Fecal Excretion of | Fecal samples will be collected from all animals predose, |
| SYN-PKU-2002 (non-GLP) | Days 7, 14, 21, 28, and from recovery animals Days 30, 35 and 40 |
| | and SYN-PKU-2002 DNA will be quantified using a qPCR method |
| | (Taqman chemistry) that uses primers specific for SYN-PKU-2002 |
| Gross | All animals, standard organ list |
| Necropsy/Organ Weights | |
| Histopathology | All animals, standard tissue list, evaluate control and high |
| | dose animals, read down when findings are seen. |
| Toxicokinetics | A sample of blood will be collected on Day 28 just prior to |
| | necropsy and analyzed for the presence of SYN-PKU-2002 DNA |
| | using a qPCR method (Taqman chemistry) that uses primers |
| | specific for SYN-PKU-2002 |

### 24. Example 71 Assessment of Gastric Phenylpyruvate in a Pig Model

Levels of gastric phenylpyruvate in two pigs (which had a duodenal cannula) at various times prior and post administration of SYN-PKU-2002.

Two days prior to administration of SYN-PKU-2002, two pigs were put on liquid diet with protein shake/apple juice for 2 days. On day 0, pigs were anesthetized and intubated, and ˜250 ml (˜50 g) Peptone, 3×10e12 bacteria (SYN-PKU-2001 in 30 ml)+24 ml 1M bicarbonate flush (2 g) were instilled at T=0.

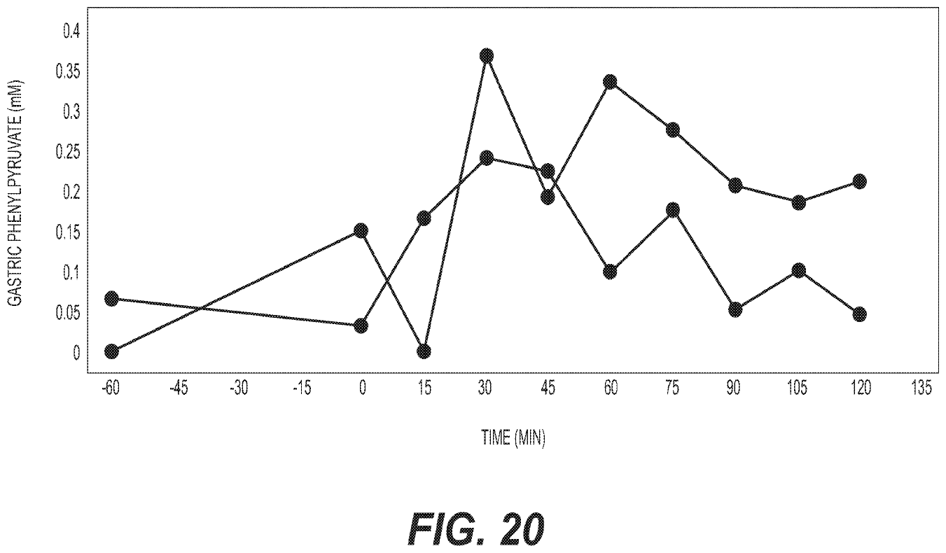

Next, 1 ml gastric samples were taken at T=0, 15 min, 30 min, 45 min, 60 min, 75 min, 90 min, 105 min, 120 min. Samples were immediately spun down, the supernatant frozen and the tube with the pellet put at 4 C. Additionally, 1 ml blood samples were taken at T=0, 30 min, 60 min, 90 min, and 120 min, collected in heparinized tubes, spun, plasma collected and frozen. When possible, urine was collected and frozen. Results are shown in FIG. 20 and indicate that LAAD is active in the stomach.

### Example 72. Conversion Efficiency of Oral Trans-Cinnamate to Urinary Hippurate in Non-Human Primates

Studies of SYN-PKU-2002 activity and efficacy were next extended to fasted healthy non-human primates (NHPs), a translational model with more relevant metabolism and GI physiology to the human. A cohort of 6 Cynomolgus monkeys (2.5-4 kg) was used. Though not a PKU phenotype, healthy NHP physiology is helpful for informing future clinical studies that will likely initially be performed in healthy humans.

All NHP studies described herein were performed at Charles River Labs (Shrewsbury, MA) in compliance with all applicable sections of the Final Rules of the Animal Welfare Act regulations (Code of Federal Regulations, Title 9), the Public Health Service Policy on Humane Care and Use of Laboratory Animals from the Office of Laboratory Animal Welfare, and the Guide for the Care and Use of Laboratory Animals from the National Research Council. Six male NHP subjects aged 2 to 5 years were used (2.5-4 kg), and were maintained on International Certified Primate Chow (PMI nutrition, 5048). Subjects were fasted overnight (16 h) before initiation of all studies. For all studies, immediately prior to dosing, animals were separated into individual cages and an angled pan was inserted at the bottom of each cage to aid in the collection of urine. Orogastric tubes were used for all oral dosing.

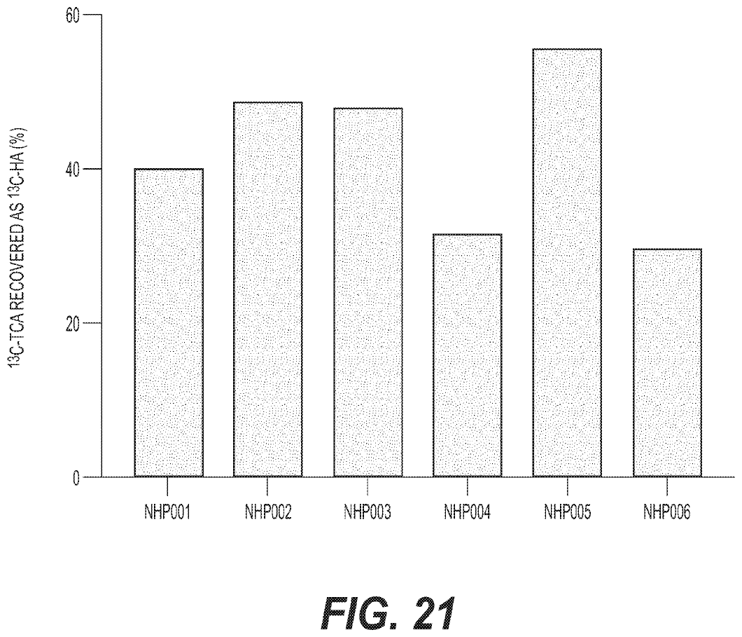

First, the conversion efficiency of orally administered TCA to urinary HA in monkeys was measured for calculation of a normalization factor to use in subsequent experiments. Each NHP was orally administered 10 mL of Peptone from meat (500 g/L; Sigma, 70174) and 15 mL of ¹³C-TCA (12.5 mg/mL; Cambridge Isotopes Lab, CLM-7498-PK) dissolved in 120 mM sodium bicarbonate, followed by a 2 mL water flush. In this manner, the in vivo PAL/Phe degradation activity of SYN-PKU could be inferred from urinary HA recovery. Isotopically labeled ¹³C-TCA was orally administered and urinary ¹³C-HA was measured. An average of 41.9±10.3% of the orally administered ¹³C-TCA was recovered as urinary ¹³C-HA (FIG. 21).

### Example 73. Profiling and Efficacy in Non-Human Primates (NHPs)

Efficacy of SYN-PKU-2002 in a non-human primate model was assessed.

In all studies described herein animals were separated into individual cages and an angled pan was inserted at the bottom of each cage to aid in the collection of urine immediately prior to dosing. Orogastric tubes were used for all oral dosing.

NHPs were orally administered 10 mL of Peptone from meat (500 g/mL) or water as a mock. Next, NHPs were administered 10 mL of SYN-PKU-2002 resuspended in formulation buffer (previously grown in activated in a bioreactor and thawed on ice) or formulation buffer alone as a mock. Finally, NHPs were administered 5 mL of 0.36M sodium bicarbonate followed by a flush with 2 mL of water. Where applicable, 1 h post dosing regimen, animals were injected intravenously with 12.5 mL of ¹³C-Phe (20 mg/mL). Animals were bled at 0, 0.5, 1, 2, 4, and 6 h by venipuncture. Where applicable, animals were given 3.5 mL of d₅-Phe (20 mg/mL; CDN Isotopes, D-1589) following mock protein dose. At 6 h post dosing, the urine collection pan was removed and the contents poured into a graduated cylinder for volume measurement. All samples were stored at −80° C. until LC-MS/MS analysis.

Phe area under the curves (AUC) were calculated with the linear-trapezoidal method using R and the PKNCA package (Denny W, D. S., and Buckeridge C. Simple, automatic, noncompartmental analysis: The PKNCA R package. J Pharmk PharmD 42.1, 11-107, doi:10.1007/s10928-015-9432-2 (2015)), and models to describe the AUCs were estimated with the rstanarm package. AUC0-last for d₅-Phe were calculated with the linear-up/log-down method. Mean and credible intervals for labeled Phe AUC and the treatment difference were calculated using a hierarchical Bayesian model with a fixed effect for treatment (vehicle or cells) and a random effect per animal.

For growth and induction of strains in bioreactors, a sterile loop was used to inoculate cells in 50 mL of FM2 medium (Supplementary Table 2) in a 500 mL Ultra-Yield™ flask (Thomson). Cells were grown at 37° C. with shaking at 350 rpm until an OD₆₀₀ of ˜5 was reached, at which point 30 mL of the culture was used to inoculate 4 L of FM2 in an Eppendorf BioFlow 115 bioreactor (starting OD₆₀₀ of ˜0.02). The fermenter was controlled at 60% dissolved oxygen with agitation, air, and oxygen supplementation, and controlled to pH 7 using ammonium hydroxide. At OD₆₀₀ of ˜1.5, cells were activated by creation of a low oxygen environment (10% dissolved oxygen), and the addition of IPTG (1 mM). At OD₆₀₀ of ˜20, L-arabinose (0.15% final concentration) was added and the cells were grown for an additional hour. Cells were harvested by centrifugation at 4,500×G for 30 min at 4° C., resuspended in formulation buffer, and stored at −80° C. until the day of testing.

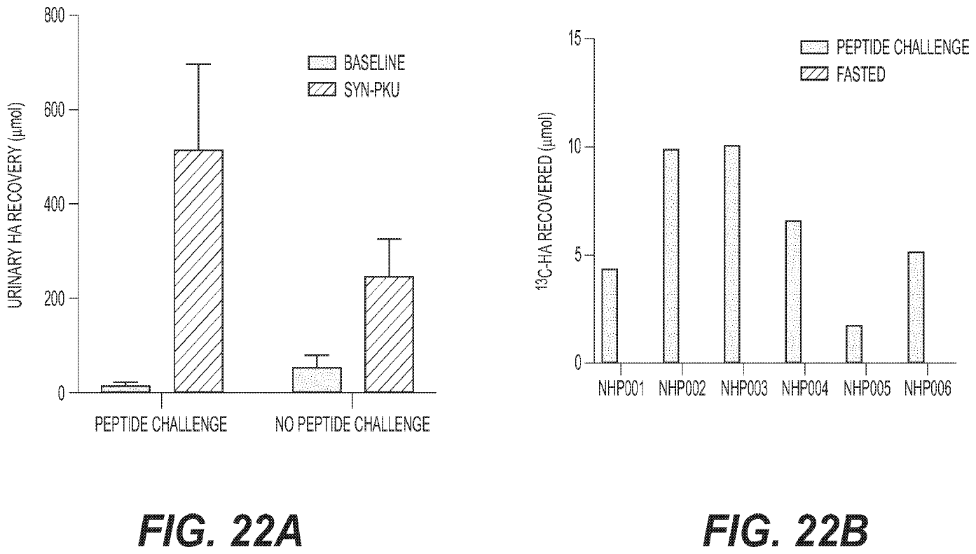

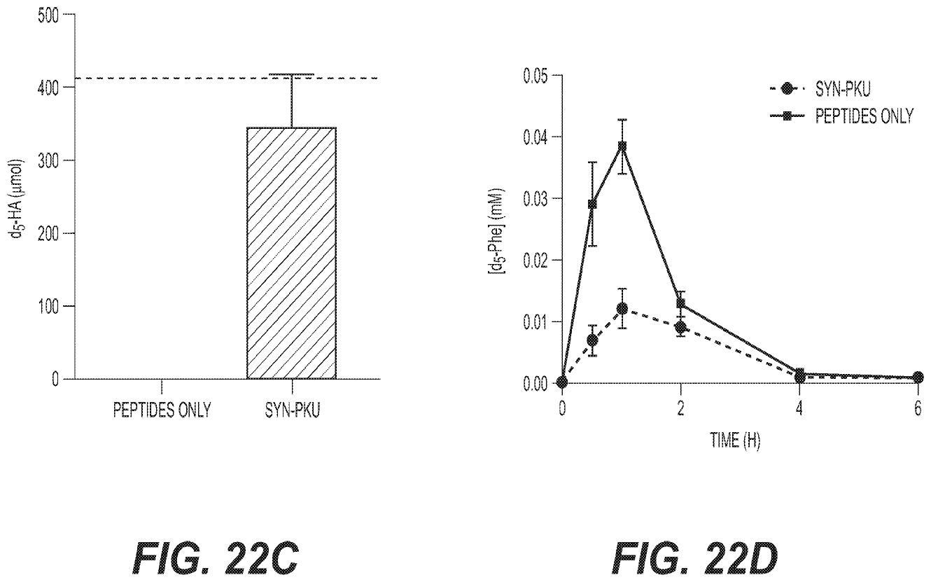

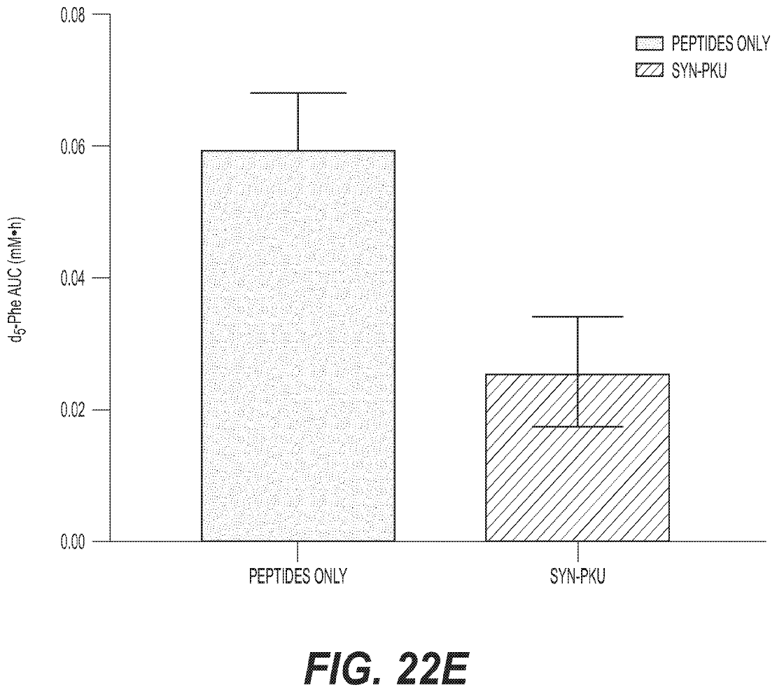

First, fasted monkeys were administered a 5 g peptide challenge with and without SYN-PKU-2002 administration and urinary HA was measured. All animals showed a significant HA response when treated with SYN-PKU-2002 compared to baseline levels without cell administration (FIG. 22A, left). Interestingly, significant HA was recovered in fasted monkeys that did not receive a peptide challenge (FIG. 22A, right), suggesting that, the primate GI tract may be a reservoir for Phe. In both preceding experiments (FIG. 22A), to examine the process of enterorecirculation in monkeys, subjects that received SYN-PKU-2002 were also injected with ¹³C-Phe intravenously 1 h following peptide challenge and ¹³C-HA was measured in urine (FIG. 22B). No ¹³C-HA was detected in the group that did not receive peptide, however, ¹³C-HA was detected in the group administered a peptide challenge, demonstrating that enterorecirculation occurs in primates, is associated with protein intake, and can provide Phe that can be consumed by SYN-PKU-2002.

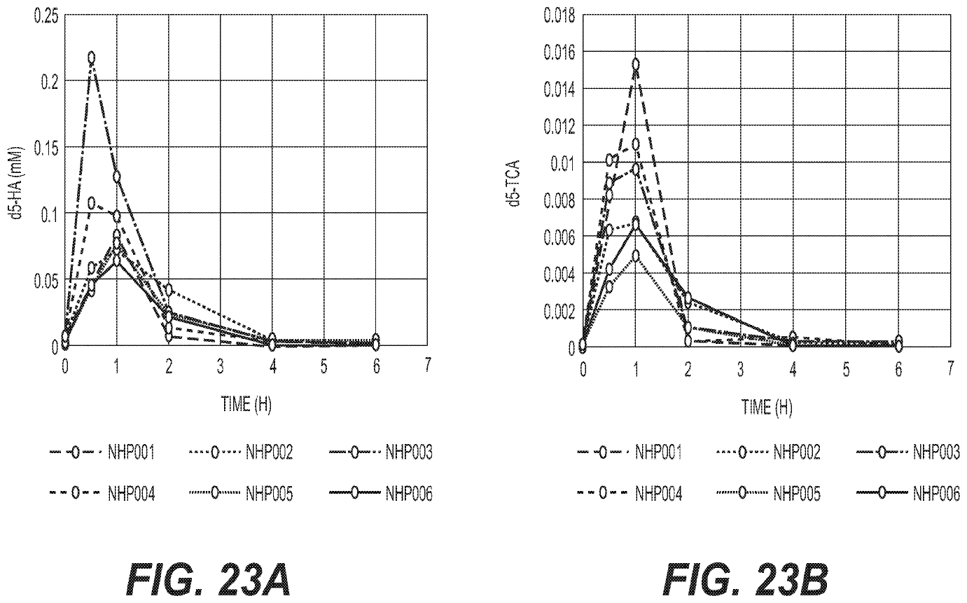

In the definitive study, the ability of SYN-PKU-2002 to lower serum Phe in monkeys was determined. However, this is difficult in healthy subjects (non-PKU), as high concentrations of protein are required to even slightly elevate serum Phe levels (by body mass, the 5 g peptide challenge used in monkeys equates to ˜100 g challenge in an average adult male). Baseline Phe levels and its low dynamic response in serum post peptide challenge due to a functional PAH enzyme obfuscates detection of serum Phe lowering by SYN-PKU-2002. For these reasons, an oral deuterated Phe (d₅-Phe) challenge was performed. The quantity of d₅-Phe administered (70 mg) equated by body mass to the Phe contained in ˜30 g protein for an average adult human male, consistent with an amount that may be present in a typical meal (Layman, D. K. Dietary Guidelines should reflect new understandings about adult protein needs. Nutr Metab (Lond) 6, 12, doi:10.1186/1743-7075-6-12 (2009)). As expected, baseline d₅-Phe was undetectable in urine and serum but could be detected in blood following its administration (FIGS. 22C and D). Following SYN-PKU-2002 administration, normalized d₅-HA recovery in the urine demonstrate that the majority of the d₅-Phe administered was metabolized to d₅-HA (FIG. 22C). SYN-PKU-2002-specific metabolites d₅-HA and d₅-TCA were also detected in serum (FIGS. 23A and B). Most importantly, an increase in serum d₅-Phe was remarkably blunted upon SYN-PKU administration (FIG. 22B), concomitant with a highly significant decrease in the AUC of serum d₅-Phe (FIG. 22C; 58% decrease with a 90% credible level between 29.4 and 57.8%).

High resting Phe levels in the GI tract of monkeys can also be inferred from the significant HA recovery observed in fasted animals that received SYN-PKU-2002 but no peptide challenge. Enterorecirculation was also shown to exist in monkeys, though difficulty working in a non-PKU background and inability to sample intestinal effluents easily in these animals indicates that more work must be performed to understand the mechanistic basis of this process. Regardless, maintenance of a reservoir of substrate may increase the therapeutic utility of SYN-PKU-2002, and impose less constraints on the timing of therapeutic administration with meals in PKU patients.

### Example 74. Assessment of LAAD in Expression of Phe in Non-Human Primates

Efficacy of of LAAD expression and determination of any negative effects on PAL metabolism of Phe was assessed.

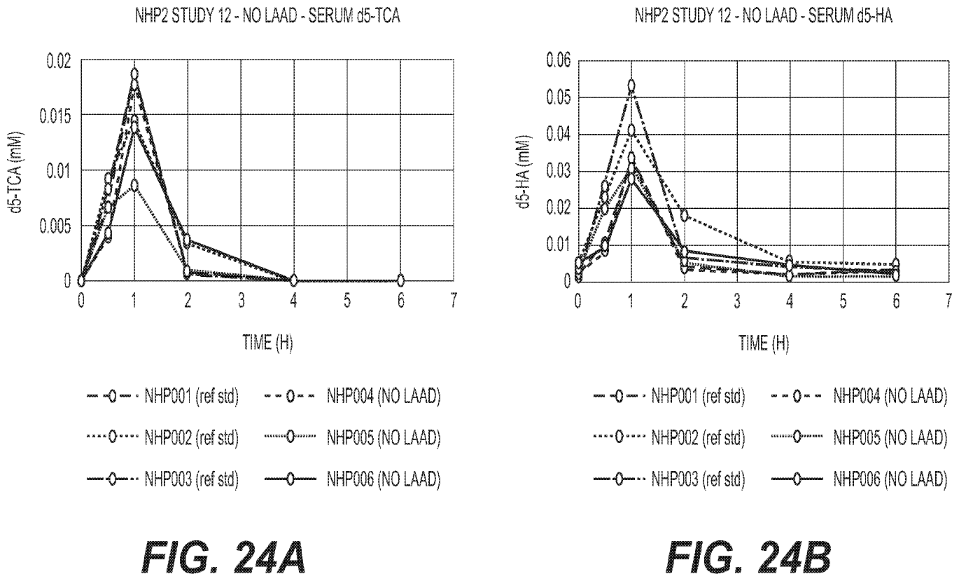

At T=0, the urine pan was emptied, and Non-Human Primates (NHPs) were orally administered 5.5 g of Peptone from meat in 11 mL, and 10 mL of an oral gavage bacteria. A SYN-PKU-2001 (5×10¹¹ CFU) oral gavage bacteria strain was administered to NHP's 1-3. A SYN-PKU-2001 (5×10¹¹ CFU) without LAAD was administered to NHP's 4-6. Both strains were suspended in formulation buffer (previously grown in activated in a bioreactor and thawed on ice) or formulation buffer alone as a mock. Concurrently, NHP's 1-10 were all administered 5 mL of 0.36M sodium bicarbonate followed by a flush with 5 mL of water. Animals were bled at 0, 0.5, 1, 2, 4, and 6 h by venipuncture. At 6 h post dosing, the urine collection pan was removed and the contents poured into a graduated cylinder for volume measurement of 5 mL. Results are shown in FIG. 24A and FIG. 24B confirm that expression of LAAD did not have a negative effect on PAL metabolism of Phe.

### Example 75. Oral Tracer Studies with Non-Human Primates (NHPs)

Oral Tracer studies were conducted to further characterize activity. At T=0, the urine pan was emptied, and NHPs were administered an oral gavage of 5.5 g of Peptone from meat in 11 mL, 4 mL D5-phenylalanine (20 mg/mL), and 10 mL of bacteria (5.2×10¹¹ CFU SYN-PKU-2001). Concurrently, NHP's 1-10 were all administered 5 mL of 0.36M sodium bicarbonate followed by a flush with 2 mL of water. Animals were bled at 0, 0.5, 1, 2, 4, and 6 h by venipuncture. At 6 h post dosing, the urine collection pan was removed and the contents poured into a graduated cylinder for volume measurement of 5 mL. FIG. 25 shows the large spike in blood Phe levels upon administration.

For the oral tracer study control, at T=0, the urine pan was emptied, and NHPs were administered an oral gavage of 5.5 g of Peptone from meat in 11 mL, 4 mL D5-phenylanaline (20 mg/mL), and 10 mL formulation buffer. Concurrently, all NHP's were administered 5 mL of 0.36M sodium bicarbonate followed by a flush with 2 mL of water. Animals were bled at 0, 0.5, 1, 2, 4, and 6 h by venipuncture. At 6 h post dosing, the urine collection pan was removed and the contents poured into a graduated cylinder for volume measurement of 5 mL.

Results in FIG. 25 show administration of the oral gavage with the SYN-PKU-2002 effectively metabolizes Phe and reduces the spike in blood Phe levels observed in the control dosage.

### Example 76. Dose Dependent Responses of SYN-PKU-2002

Four studies confirmed successful dose dependent conversions of Phe and production of plasma biomarkers t-cinnamic acid (TCA) and hippuric acid (HA) in non-human primates (NHPs) after single dose administration of SYN-PKU-2002 after a protein rich meal.

Study 1: At T=0, the urine pan was emptied, and ten NHPs were orally administered 5.5 g of peptone from meat in 11 mL of water. NHP's 1-5 were administered an oral gavage of 4 ml of SYN-PKU-2002 (3.6×10¹¹ CFU). NHPs 6-10 were administered an oral gavage of 4 mL formulation buffer, 2 mL of SYN-PKU-2002 (1.8×10¹¹ CFU) followed by an oral gavage of 6 mL of formulation buffer. Concurrently, NHP's 1-10 were all administered 5 mL of 0.36M sodium bicarbonate followed by a flush with 5 mL of water. Animals were bled at 0, 0.5, 1, 2, 4, and 6 h by venipuncture. At 6 h post dosing, the urine collection pan was removed and the contents poured into a graduated cylinder for volume measurement of 5 mL.

Study 2: Ten NHP's were orally administered 5.5 g of Peptone from meat in 11 mL. NHP's 1-5 were gavaged with 8 ml of SYN-PKU-2002 (7.2×10¹¹ CFU) bacteria strain. NHP's 6-10 were gavaged with 1 mL of SYN-PKU-2002 (9.0×10¹⁰ CFU) followed 7 mL of formulation buffer. Concurrently, NHP's 1-10 were all administered 5 mL of 0.36M sodium bicarbonate followed by a flush with water. Animals were bled at 0, 0.5, 1, 2, 4, and 6 h by venipuncture. At 6 h post dosing, the urine collection pan was removed, and the contents poured into a graduated cylinder for volume measurement of 5 mL.

Study 3: Ten NHP's were orally administered 5.5 g of Peptone from meat in 11 mL. NHP's 1-5 were gavaged with 8 ml of SYN-PKU-2002 diluted in formulation buffer (2.3×10¹⁰ CFU). NHP's 6-10 were gavaged with 8 mL of diluted SYN-PKU-2002 diluted in formulation buffer (4.5×10¹⁰ CFU). Concurrently, NHP's 1-10 were all administered 5 mL of 0.36M sodium bicarbonate followed by a flush with 5 mL of water. Animals were bled at 0, 0.5, 1, 2, 4, and 6 h by venipuncture. At 6 h post dosing, the urine collection pan was removed and the contents poured into a graduated cylinder for volume measurement of 5 mL. NHP's 1-10 all fasted the previous night.

Study 4: Ten NHP's were orally administered 5.5 g of Peptone from meat in 11 mL. NHP's 1-5 were gavaged with 8 ml of diluted SYN-PKU-2002 (3.3×10⁹ CFU). NHP's 6-10 were gavaged with 8 mL of diluted SYN-PKU-2002 (1.1×10¹⁰ CFU). Concurrently, NHP's 1-10 were all administered 5 mL of 0.36M sodium bicarbonate followed by a flush with 5 mL of water. Animals were bled at 0, 0.5, 1, 2, 4, and 6 h by venipuncture. At 6 h post dosing, the urine collection pan was removed and the contents poured into a graduated cylinder for volume measurement of 5 mL. NHP's 1-10 all fasted the previous night.

Results shown in FIGS. 117A, 117B, 117C, and 117D show that Phe metabolism was evident at doses of 9×10¹⁰ CFU and higher. Additionally, dose-dependent conversion of Phe and production of biomarkers TCA and hippuric acid were observed.

### Example 77. Casein Study in Non-Human Primates with SYN-PKU-2002

Efficacy of replacing peptones (composed of small peptides) with casein (a whole protein) for expressing Phe, TCA, and HA consumption was evaluated.

At T=0, urine pans were emptied, and NHPs were administered an oral gavage of 28 mL of casein (4.5 g)/biocarbonate/D5-phenylalanine (25 mg; 8 mg/kg). NHPs 1-5 were further administered an oral gavage of 3.5 mL SYN-PKU-2002 (5×10¹¹ CFU), and NHPs 6-10 were administered 3.5 mL formulation buffer via oral gavage. Concurrently, the NHPs were administered a flush with 2 mL of water. Animals were bled at 0, 0.5, 1, 2, 4, and 6 h by venipuncture. At 6 h post dosing, the urine collection pan was removed and the contents poured into a graduated cylinder for volume measurement of 5 mL. Results are shown in FIGS. 118A-118C.

The results from this study demonstrate that the genetically engineered bacterial strains of the disclosure can consume Phe that is naturally digested and can prevent a spike in blood Phe observed in the control upon D5-Phe consumption.

### Example 78. Phe Metabolism is Clinically Relevant

The disclosed studies in non-human primates demonstrate the relevancy of Phe metabolism and the profound effect it could have on the diet of patients with PKU. The Institute of Medicine has established dietary reference intakes that establish a phenylalanine is assumed to be 80-90 mg/g whole protein. The recommended intake of protein for a human is 50 g (for a 2000 calorie diet). An unrestricted American diet provides approximately 100 g of whole protein (males) and 70 g (female) on average. An unrestricted diet will therefore provide approximately 6-9 g of phenylalanine.

The recommended phenylalanine consumption for patients with PKU>4 years of age is 200-1100 mg (Vockley, et al. 2014, the entire contents of which are incorporated herein by reference), or about <10 g protein/day. Based on the studies above, the highest dosage of SYN-PKU-2002 can be extrapolated (assuming 35% recovery of urinary HA in NHP's) to three 5×10¹¹ doses per day in patients. Said dosage predicts the daily metabolism of Phe to support a ˜25 g of dietary protein intake in humans (25 mg/kg=1.7 g for a 70 kg person). Therefore, the dose of Phe is about 25% of the Phe intake that a PKU patient following an unrestricted diet would receive in 24 hours (FIG. 28). This is ˜50% of the daily recommended protein intake (see US RDA; assuming an unrestricted American diet provides on average approximately 100 g (males) and 70 g (females) of whole protein). For PKU patients, this results an a 2.5-fold increase in protein uptake, a result which would liberate the diet and lifestyle of PKU patients.

In some embodiments, upon treatment with the bacteria of the disclosure, PKU patients may increase their dietary protein intake per day to greater than 10 g; from about 10 g to about 30 g, from about 10 g to about 25 g, from about 10 g to about 20 g, from about 10 g to about 15 g. In some embodiments, upon treatment with the bacteria of the disclosure, PKU patients may increase their dietary protein intake per day to about 15 g, about 20 g, or about 25 g.

In some embodiments, upon treatment with the genetically engineered bacteria of the disclosure, PKU patients may increase their daily protein intake by about 2.5 fold. In some embodiments, upon treatment with the genetically engineered bacteria, PKU patients may increase their daily protein intake by about 2.0 fold. In some embodiments, upon treatment with the genetically engineered bacteria, PKU patients may increase their daily protein intake by about 1.5 fold In some embodiments, upon treatment with the genetically engineered bacteria, PKU patients may increase their daily protein intake by about 1.0 fold. In some embodiments, upon treatment with the genetically engineered bacteria, PKU patients may increase their daily protein intake by about 3 fold, 3.5 fold or 4 fold. In some embodiments, upon treatment with the genetically engineered bacteria, PKU patients may increase their daily protein intake by about 50%. In some embodiments, upon treatment with the genetically engineered bacteria, PKU patients may increase their daily protein intake by about 5% to 10%, 10% to 20%, 20% to 30%, 30% to 40%, 40% to 50%, 50% to 60%, 60% to 70%, 70% to 80%, 80% to 90%, 90% to 100%. In some embodiments, upon treatment with the genetically engineered bacteria, PKU patients may increase their daily protein intake by about 5%, 10%, 20%, 30%, 40%, 50%, 60%, 70%, 80%, and 90% to 100%.

### Example 79: Characterization of the Growth of SYN766, an Escherichia coli Nissle 1917 DAP Auxotroph in Varying Concentrations of Diaminopimelate

To determine the minimum amount of DAP required for bacterial growth and division, the growth of SYN766, an EcN DAP auxotroph, was characterized by incubation in a range of DAP concentrations.

All bacterial cultures were started from glycerol stocks stored at ≤−65° C. Bacteria were grown in 14 mL culture tubes overnight with shaking at 250 rpm at 37° C. in LB broth supplemented with DAP at 100 μg/mL. Overnight cultures were then diluted 1:100 in wells of 96-well plates containing 100 μL of LB with various DAP concentrations.

In all experiments, the 96-well plates were sealed with parafilm and analyzed on a Synergy™ Neo Microplate Reader (BioTek Instruments, Inc., Winooski, VT). The plate was incubated at 37° C. with shaking for 960 minutes, and the OD₆₀₀ was measured every 10 minutes.

The data were collected in Gen5™ software version 2.06 (BioTek Instruments, Inc., Winooski, VT), exported to Microsoft® Excel to calculate the mean and standard error, and processed in GraphPad Prism version 7.03 (GraphPad Software, Inc., San Diego, CA).

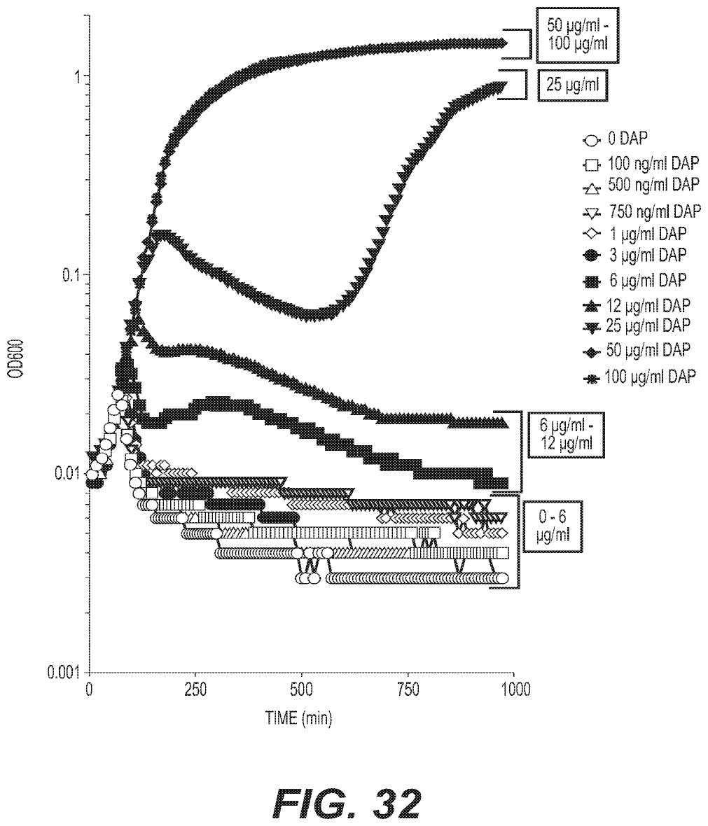

SYN766, a DAP auxotroph strain of EcN, was analyzed to determine the concentration of DAP required for growth; the data are displayed in FIG. 32. In liquid media with DAP at concentrations of 50 μg/mL and 100 μg/mL, SYN766 grew exponentially for 350 minutes before entering stationary phase at an OD₆₀₀ of approximately 1. At a DAP concentration of 25 μg/mL, cells grew for 200 min, reaching an OD₆₀₀ of 0.15 before declining. Growth resumed after 600 min and reached a final OD₆₀₀ of approximately 0.8, which was significantly less total cell mass than when the DAP concentration was at least 50 μg/mL. At 6 μg/mL and 12 μg/mL DAP, SYN766 grew exponentially for approximately 180 minutes before displaying a declining OD₆₀₀ for the remainder of the experiment. When DAP concentrations were below 6 μg/mL, SYN766 showed growth for approximately 100 min before displaying a declining OD₆₀₀ for the remainder of the experiment.

### Example 80: Characterization of the Growth of Various Strains of Escherichia coli Nissle 1917 in LB Growth Media without Diaminopimelate

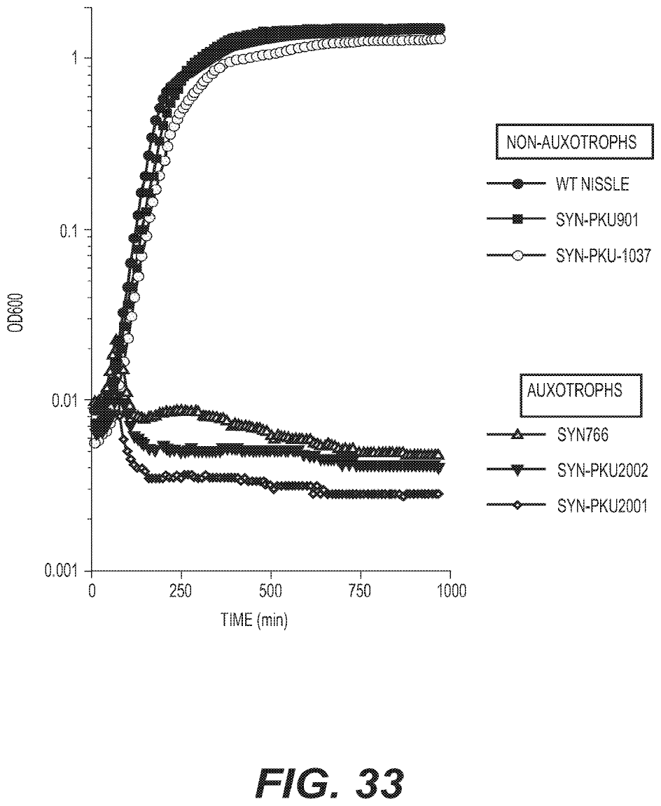

Various modified strains of EcN were grown in media without DAP to confirm the growth characteristics of DAP auxotrophs and prototrophs. Similar growth conditions were used to those described in Example 79. Various strains of EcN that have been modified to consume phenylalanine were analyzed for their ability to grow in the absence and presence of DAP. Results are shown in FIG. 33. The non-auxotrophic strains, SYN001, SYN-PKU901 and SYN3282, grew exponentially for approximately 400 minutes before entering stationary phase at an OD600 of approximately 1.2. Under similar conditions, the DAP auxotroph strains, SYN766, SYN-PKU-2001, and SYN-PKU-2002, stayed within the inoculation OD600 of approximately 0.010 for the first 90 min before starting to show a decline, concluding the experiment at an OD600 of approximately 0.005. This shows that the deletion of dapA results in a cessation of growth in the absence of an external source of DAP. The presence of genes involved in Phe degradation in SYN3282 had no effect on growth compared to wild type strains. The combination of DAP auxotrophy with genes involved in Phe degradation in SYN-PKU-2001 and SYN-PKU-2002 had no effect on the growth impairment observed with DAP auxotrophy alone (SYN766). These results show that DAP auxotrophy is the main factor regulating growth and survival of these engineered strains.

### Example 81: Characterization of the Growth of Various Strains of Escherichia coli Nissle 1917 in LB Growth Media with 100 μg/mL Diaminopimelate

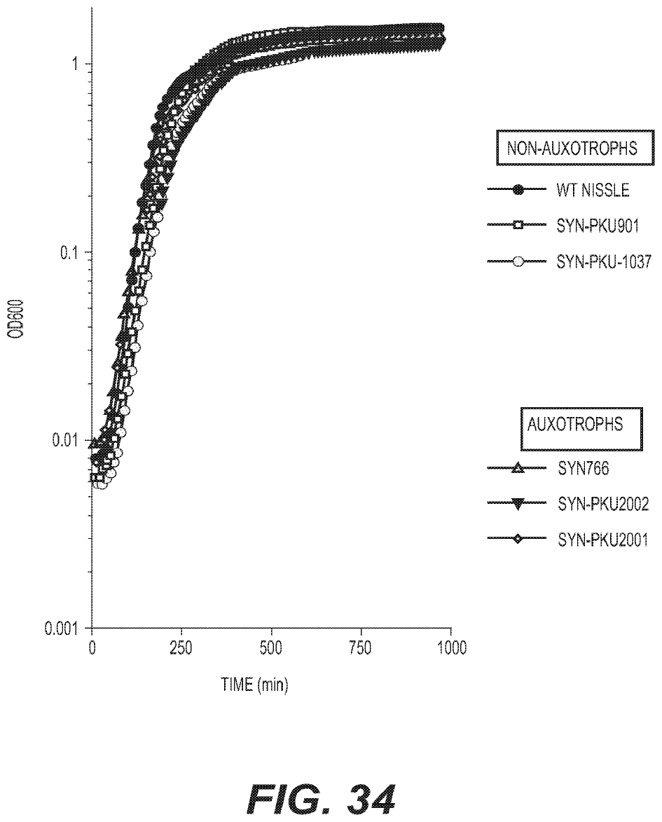

Various modified strains of EcN were grown in media with 100 μg/mL DAP, using identical growth conditions to those described in Example 79. In contrast to the results in example 80, when grown in LB supplemented with 100 μg/mL DAP, all of the tested strains of EcN exhibited comparable growth (see FIG. 34). The non-auxotrophic strains SYN001, SYN-PKU901 and SYN3282 and the auxotrophic strains SYN766, SYN-PKU-2001 and SYN-PKU-2002 grew exponentially for approximately 400 minutes before entering stationary phase at an OD₆₀₀ of approximately 1.4.

### Example 82: Fecal Excretion and Clearance of Engineered Escherichia coli Nissle 1917 in C57BL/6 Mice Following Oral Administration

The major objective of this experiment was to determine the kinetics of fecal excretion of strains containing DAP auxotrophy and/or the Phe-degradation activity compared to wild type EcN. EcN strains utilized for this study included wild type (SYN-PKU901), a DAP auxotroph (SYN766), a strain genetically engineered to encode Phe-degrading activity (SYN3282), and a strain with both DAP auxotropy and Phe-degrading activity (SYN-PKU-2001). SYN-PKU-2001 is a surrogate for the clinical candidate strain SYN-PKU-2002, but contains a chloramphenicol resistance cassette that allows it to be isolated from other microbial flora in intestinal contents and biological tissues by plating on selective media. SYN3282 is identical to SYN-PKU-2001, but does not have DAP auxotrophy. For this purpose, female C57BL/6 mice were weighed and randomized by weight into 3 treatment groups (n=5 each). Each group would receive a single oral dose containing 2 mixed strains of bacteria at approximately 3×10⁹ CFU/strain (200 μL/dose). Group 1 received SYN-PKU901 and SYN766 to examine the effect of DAP auxotrophy, group 2 received SYN-PKU901 and SYN3282 to examine the effect of Phe-degradation activity, and group 3 received SYN-PKU-2001 and SYN3282 to examine the combined effects of both DAP auxotrophy and Phe-degradation activity.

Before dosing, fecal samples were collected from each individual mouse at T=0 and processed by serial microdilution plating to determine the baseline level of viable CFUs, if any, on LB agar plates selective for the bacterial strains to be dosed within the group. Bacterial doses were then administered immediately after the T=0 h fecal collection. For collection of feces at designated time points, each mouse was isolated in a small empty pipet tip box until a recoverable fecal sample was produced. Samples were moved into 2 mL microcentrifuge tubes. If no sample was produced within 30 minutes of isolation, the time point for that animal was excluded from analysis. Fecal pellets were collected and weighed from each mouse at 4, 6, 8, 24, 30, and 48 h post-dose.

For processing of weighed fecal pellets, 1 mL of PBS was added to each sample and disposable pellet pestles (Kimble, 749521-150) were used for manual homogenization. The samples were then vortexed at maximum speed for 10-20 s. The 10 μL microdilution was used to dilute samples 1:10 across 6 orders of magnitude. Ten (10) μL of these dilutions and the undiluted fecal homogenate were plated on the appropriate selective LB agar plates. Dilutions were plated on LB agar supplemented with antibiotics or DAP where appropriate (SYN-PKU901=streptomycin 300 μg/mL; SYN3282=chloramphenicol 30 μg/mL; SYN766/SYN-PKU-2001=chloramphenicol 30 μg/mL and DAP 100 μg/mL). Plates were incubated overnight at 37° C. and colonies were counted manually the following day. Plates were incubated overnight at 37° C.±2° C. Colonies were counted manually in a dilution that contained at least 10 but no more than 200 individual colonies. The final CFU count was determined by back-calculation using the dilution factor multiplied by the number of colonies counted for calculation of CFU/mL of fecal homogenate. This value was divided by the weight of fecal pellets for calculation of CFU/mg feces.

Next, the effect, if any, of mutation/engineering in modified EcN strains compared to their isogenic parents was examined. The amount of each strain excreted at each time point was divided by the number of CFUs of that strain in the initial dose. Using this normalization, the two strains within a study group could be directly compared for excretion and clearance kinetics.

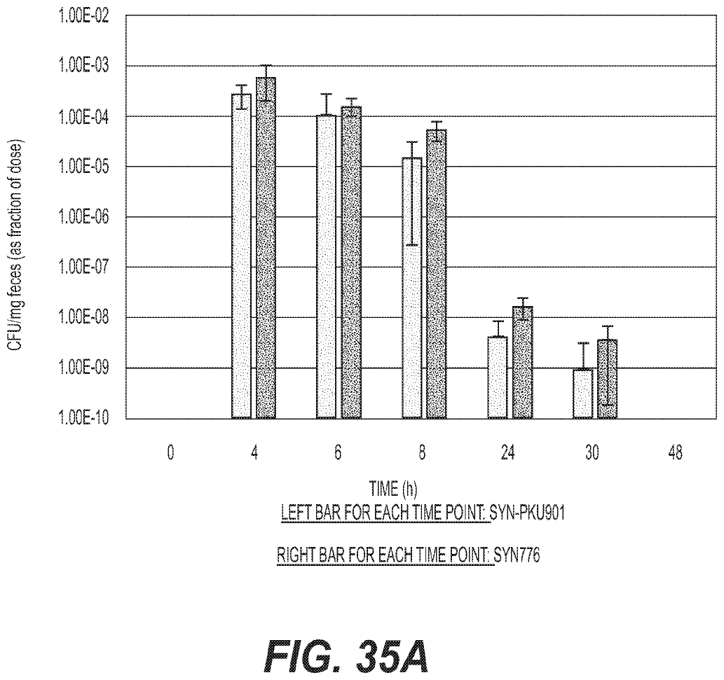

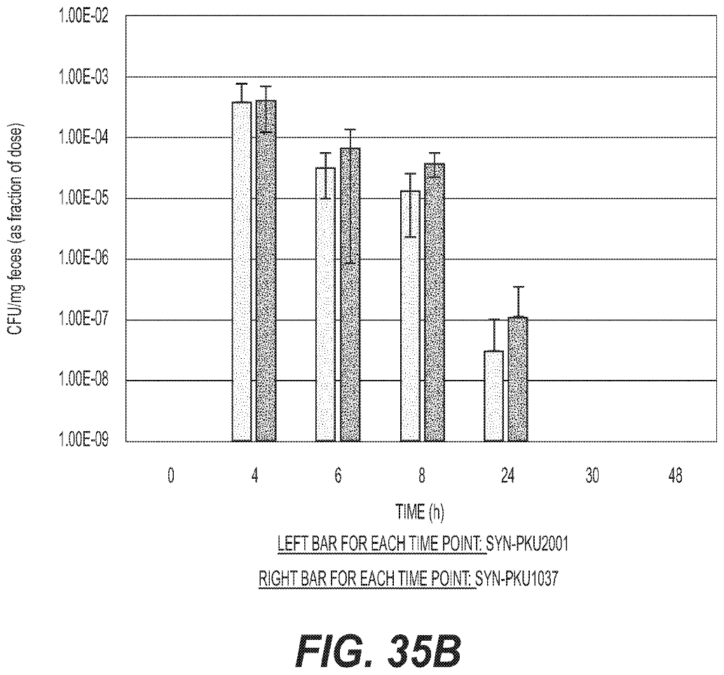

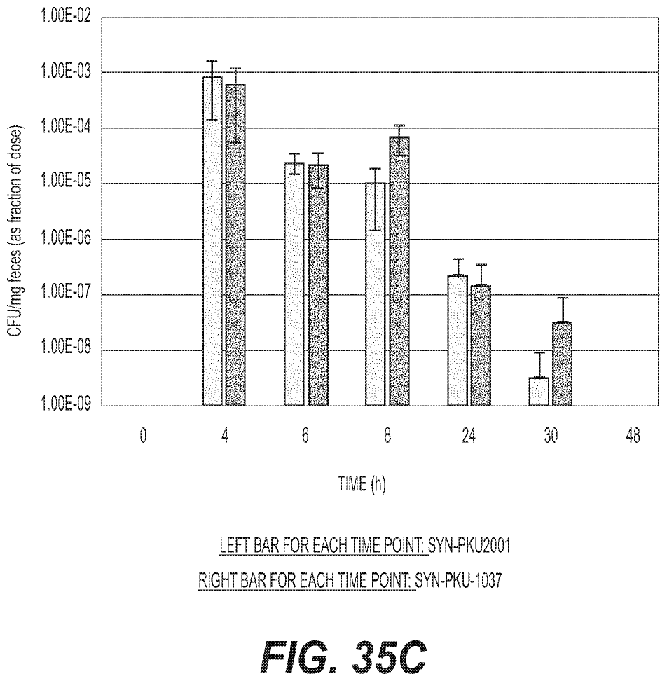

Results are shown in FIG. 35A-35C. There were no CFUs detected on selective media in the pre-dose (T=0 h) fecal samples for any of the mice, demonstrating that CFUs detected post-dosing were attributable to EcN-derived strains and not due to background growth from the microbiome. In all 3 groups, all strains of bacteria had reached peak amounts in the feces at 4 h post dose and steadily declined over time. By 24 h, bacterial counts in the feces dropped by 3-4 orders of magnitude for all strains. By 30 h, at least 2 mice from groups 1 and 3, and all mice in group 2, contained no detectable EcN-derived bacteria in the feces. By 48 h, all EcN-derived bacteria were cleared from the feces. There were no apparent differences in the kinetics of excretion in the modified EcN strains, as similar rates of elimination were observed for all bacteria, showing that neither DAP auxotropy (SYN766, group 1), Phe-degradation activity (SYN3282, group 2), nor their combination (SYN-PKU-2001, group 3), had any significant effect on intestinal transit time or strain survival in the mouse model compared to the wild type SYN-PKU901 strain.

In conclusion, DAP auxotrophy and/or Phe degradation activity in EcN did not change the survival or disappearance of bacteria from the feces of C57BL/6 mice over time. All orally administered EcN-derived bacterial strains, regardless of genotype, were cleared from the feces of mice within 48 h, suggesting that EcN and its derivatives are not viable long term.

### Example 83: In Vivo Survival of Escherichia coli Nissle 1917 Derivatives SYN-PKU901 and SYN-PKU-2001 in Healthy Mice

The objective of this pharmacology study was to evaluate in female C57BL/6 mice any differences in survival, gastrointestinal (GI) distribution or time to complete clearance of Escherichia coli Nissle (EcN) wild type strain SYN-PKU901 compared to the genetically engineered EcN derivative SYN-PKU-2001, a surrogate strain for SYN-PKU-2002, which contained identical diaminopimelate (DAP) auxotrophy and phenylalanine (Phe) degradation elements. GI distribution, transit and clearance kinetics were measured by enumerating colony forming units (CFUs) over a 48-h time period in multiple GI segments of female C57BL/6 mice following a single oral dose of 9×109 CFUs of SYN-PKU901 or SYN-PKU-2001.

EcN strains utilized for this study included SYN-PKU901, a wild type control, and SYN-PKU-2001, a strain genetically engineered to encode Phe-degrading activity and DAP auxotrophy. SYN-PKU-2001 is a surrogate for the clinical candidate strain SYN-PKU-2002, but contains a chloramphenicol resistance cassette that allows for selection plating from biological tissues.

Female C57BL/6 mice were weighed and randomized by weight into 12 treatment groups (n=3 each) and a control group (n=3). The mice in the control group (T=0) were sacrificed by carbon dioxide asphyxiation followed by cervical dislocation. The stomach, small intestine (SI), cecum, and colon were carefully excised. The SI was further divided into 3 equal length sections (upper, middle, and lower). Each organ section was flushed with 0.5 mL of ice cold phosphate-buffered saline (PBS) and effluents were collected into separate 1.5 mL microcentrifuge tubes. The PBS/effluent samples were weighed and maintained on ice until processing by serial dilution plating on selective media to determine viable CFUs in the effluent of each intestinal segment. In formulations containing 100 mM sodium bicarbonate, animals in the treatment groups received an oral dose of 9×109 CFUs of SYN-PKU901 (6 groups) or SYN-PKU-2001 (6 groups). At 0.25, 0.5, 1, 4, 24, and 48 h post dose, a SYN-PKU901- and a SYN-PKU-2001-treated group were sacrificed and tissues were processed in a manner identical to the control group.

To calculate CFUs/mL, 10 μL of each weighed effluent sample was used in a 10-fold microdilution series performed in PBS in a sterile 96-well plate. Ten (10) μL of the undiluted effluent and of the dilutions were plated on LB agar supplemented with antibiotics or DAP where appropriate (SYN-PKU901=streptomycin 300 μg/mL; SYN-PKU-2001=chloramphenicol 30 μg/mL and DAP 100 μg/mL). Plates were incubated overnight at 37° C. and colonies were counted manually the following day.

For CFU determination, counts were collected by the manual counting of colonies in a dilution that contained at least 10 but no more than 200 individual colonies. In cases where colonies were only observed at the lowest dilution (the plating of undiluted effluent), all colonies were counted and scored, even when less than 10. The final CFU count was determined by back-calculation using the dilution factor multiplied by both the number of colonies counted and the volume of effluent collected.

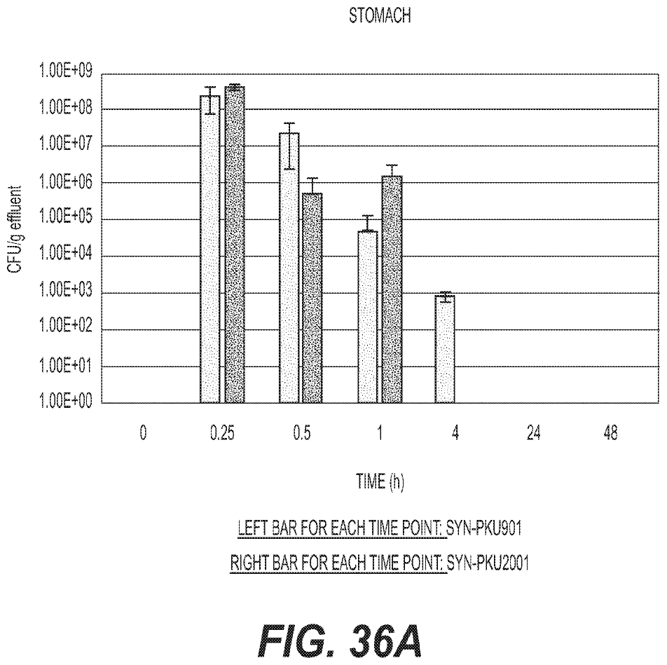

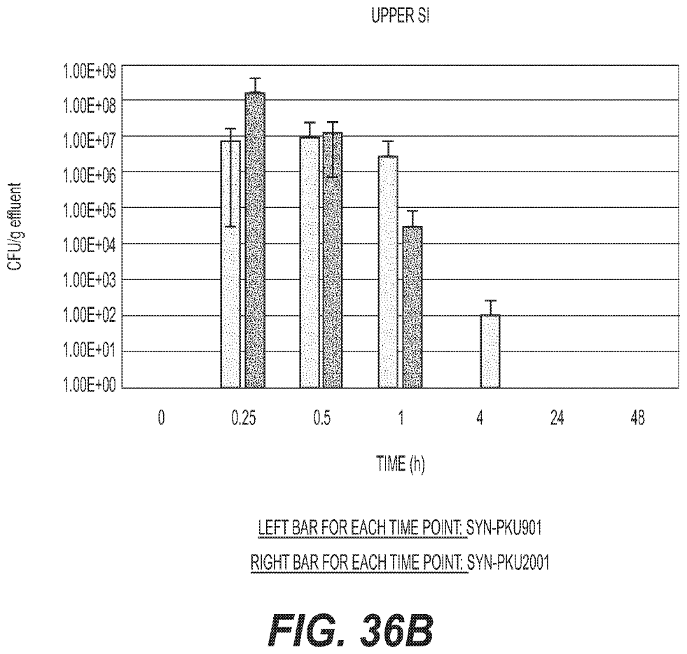

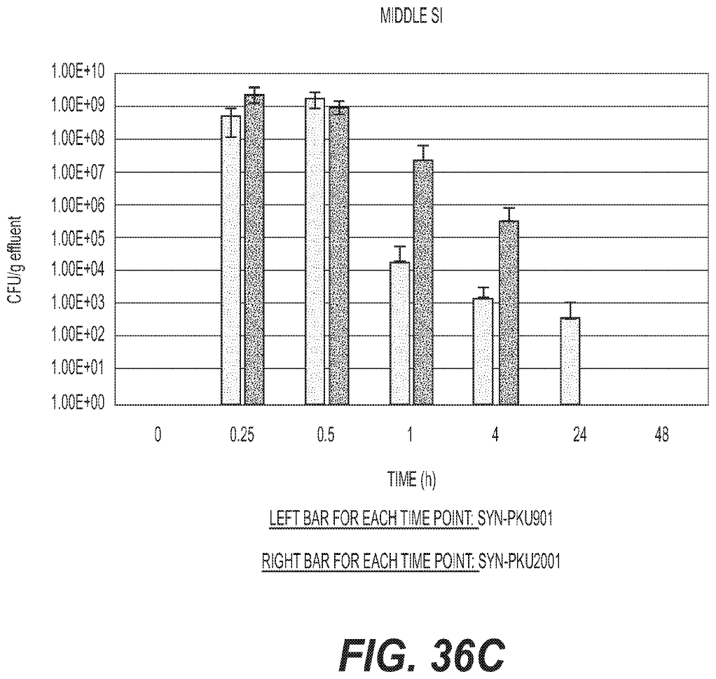

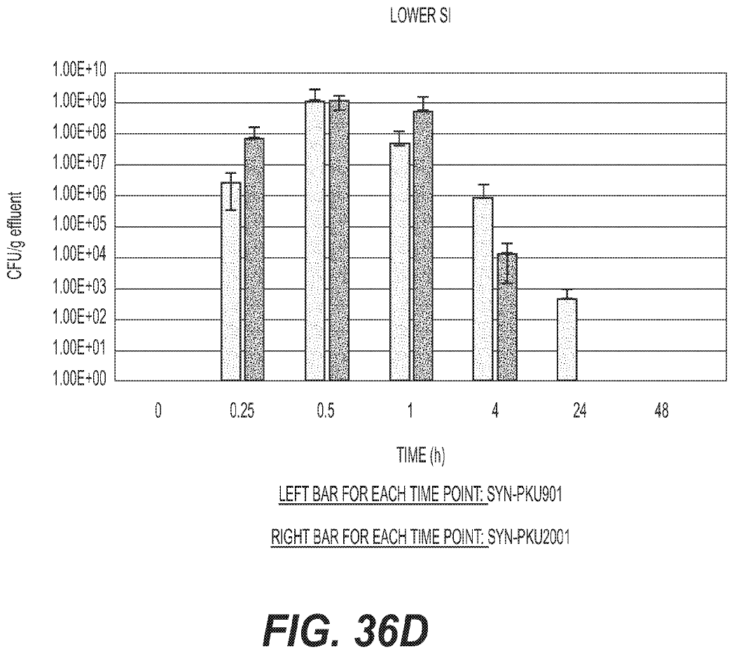

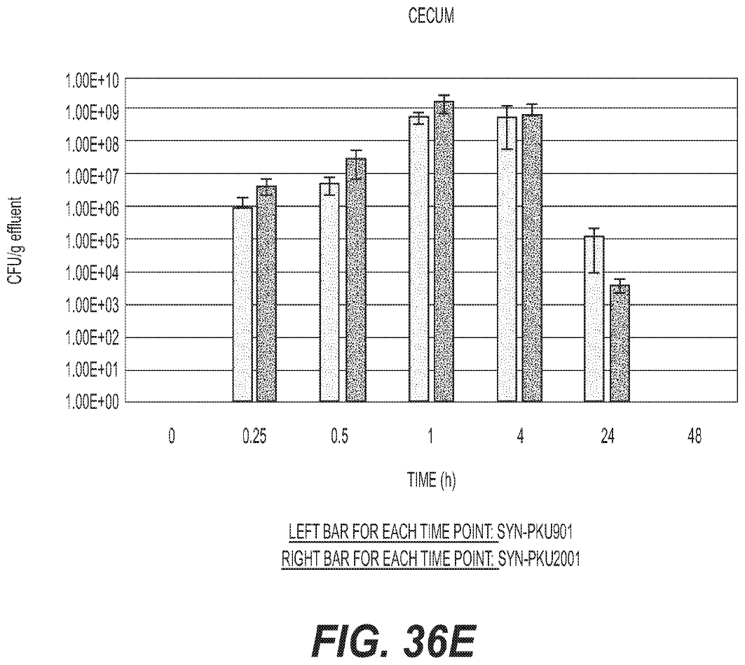

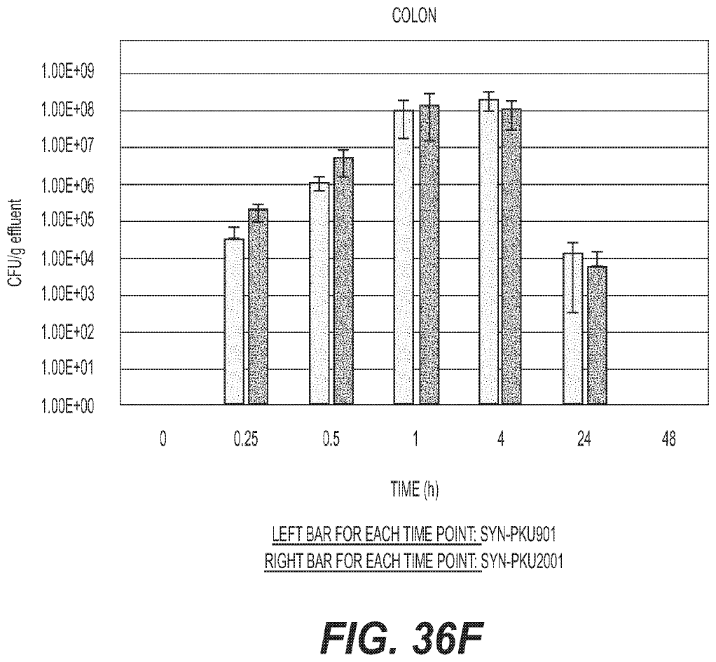

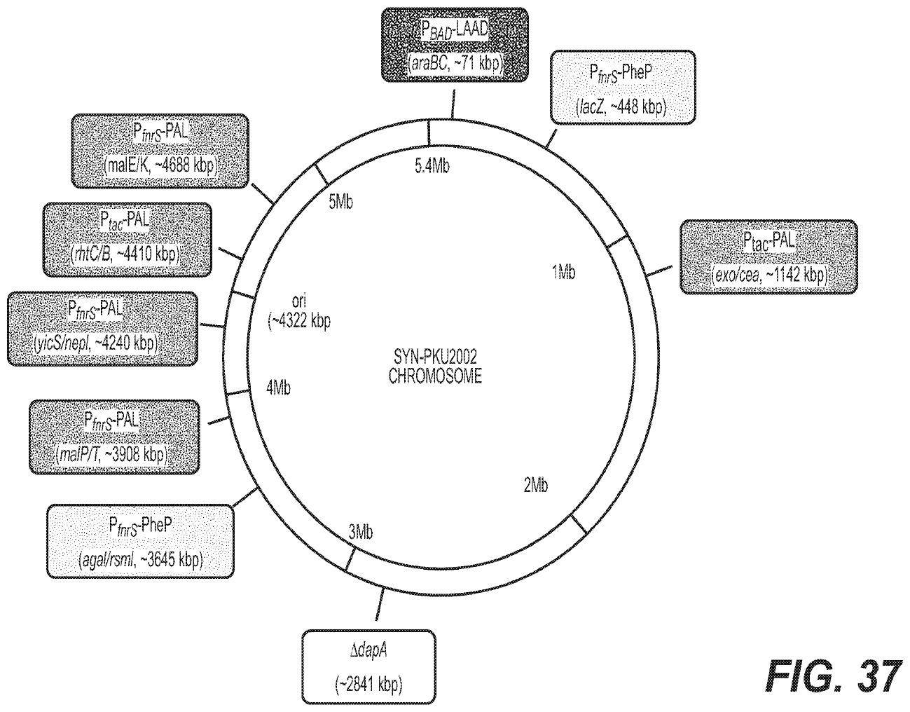

See results in FIG. 36A-F. No CFUs were detected on selective media in the effluent samples of mice that did not receive cells, demonstrating that CFUs detected in effluents post-dosing were attributable to EcN-derived strains and not due to background growth from the microbiota. In the effluents obtained from the stomach, upper small intestine, middle small intestine, lower small intestine, cecum and colon of mice dosed with either SYN-PKU-2001 or SYN-PKU901 (control), survival and GI distribution over time were highly similar over the first 24 hours. No CFUs were found for either bacteria at 48 h post-dose, demonstrating complete clearance by this time point.

In conclusion, DAP auxotrophy and/or the phenylalanine-degradation activity of SYN-PKU-2001 did not change the survival, GI distribution or time to complete clearance from the GI tract of C57BL/6 mice compared to the SYN-PKU901 control strain. The complete clearance of all orally administered EcN-derived strains within 48 h suggests that EcN and its derivatives were not viable long term.

### Example 84: Phenylalanine Degradation to Trans-Cinnamate and Phenylpyruvate in Bioreactor-Grown Escherichia coli Strain SYN-PKU-2002

The conversion of phenylalanine (Phe) into the metabolites trans-cinnamate (TCA) and phenylpyruvate (PP) in modified Escherichia coli Nissle 1917 (EcN) strain SYN-PKU-2002 that was grown and activated in a bioreactor was determined as a measure of phenylalanine ammonia lyase (PAL) and L-amino acid deaminase (LAAD) activity. Additional objectives included determining if bioreactor-activated SYN-PKU-2002 could use complex mixtures of Phe-containing peptides or whole protein as substrates for Phe degradation.

EcN-derived strain SYN-PKU-2002, a Phe-degrading strain intended for the therapeutic treatment of phenylketonuria (PKU), was grown and activated in a bioreactor following a process intended to be used for the scale-up of SYN-PKU-2002 for clinical trials. This process, performed on a 5 L scale, included the creation of a low dissolved oxygen (DO) environment that was achieved through control of culture agitation rate. Low DO activated expression of chromosomally integrated copies of genes encoding PAL and high affinity phenylalanine transporter, PheP. Following the drop to low DO, isopropyl β-D-1-thiogalactopyranoside (IPTG; 1 mM) was added to activate expression of additional copies of PAL, and L-arabinose (0.15% final concentration) was added to activate expression of LAAD. Following high density growth, cells were concentrated and frozen at ≤65 C in glycerol-based formulation buffer.

1×108 activated cells were incubated aerobically at 37 C in assay media containing 50 mM Phe, Phe-Pro, Phe-Gly-Gly, Phe-Val, Phe-Ala, or Gly-Phe peptides, or in 20 g/L of peptone from meat, tryptone, or whole casein powder. Supernatant samples were removed at 30 and 60 min and the concentrations of Phe, TCA, and PP were determined by LC MS/MS.

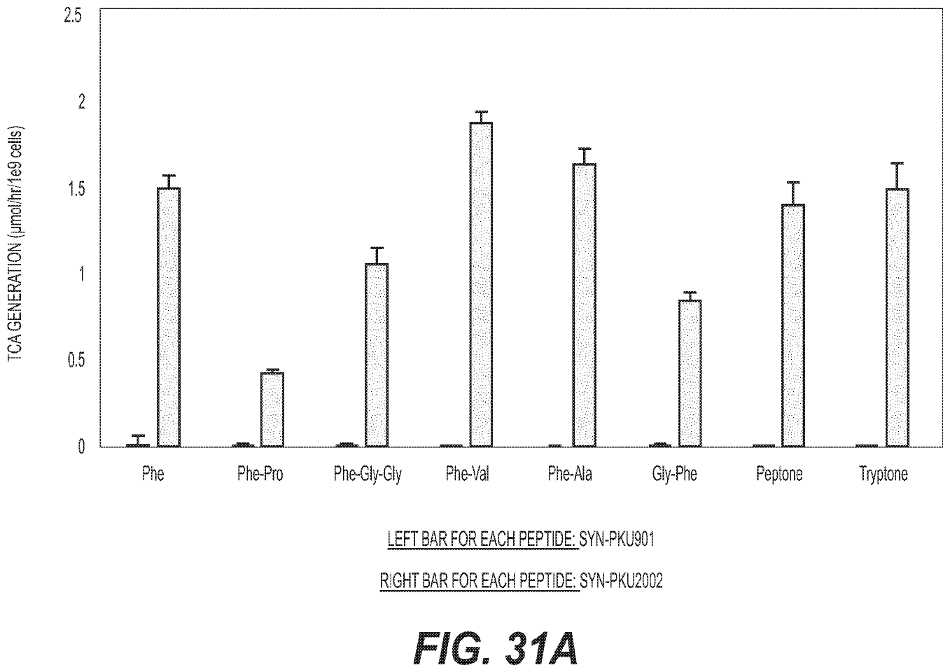

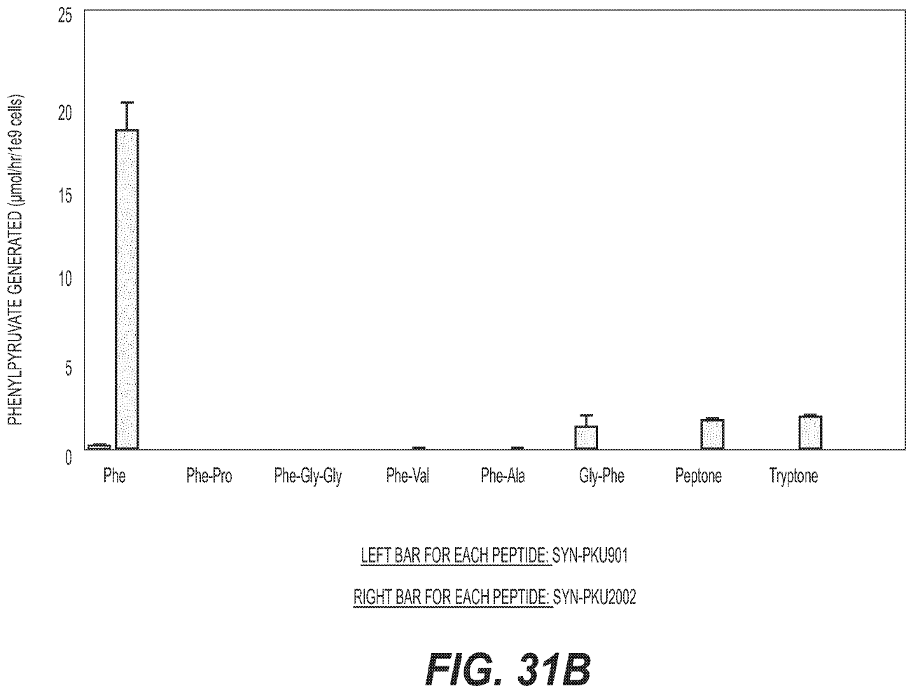

See results in FIGS. 31A and B. Although both PAL and LAAD activities in SYN-PKU-2002 could be detected in vitro when free Phe was used as the substrate, only PAL activity was observed when the provided substrate came in the form of Phe-containing peptides, demonstrating that LAAD may be better at accessing free amino acid than other sources of Phe. No PAL or LAAD activity was observed when undigested casein protein was used as a substrate.

These data demonstrate that SYN-PKU-2002 grown in a bioreactor is capable of growth and activation of both the PAL and LAAD Phe degrading pathways, suggesting that SYN-PKU-2002 is amenable to the production of activated bulk drug at higher volumes. Additionally, it was demonstrated that complex mixtures of Phe-containing peptides, likely to be produced in addition to free Phe during normal digestive processes in the mammalian gastrointestinal tract, are metabolized by the PAL component of SYN-PKU-2002 but not the LAAD component.

### Example 85: Dose Response Study of SYN-PKU-2002 in Healthy Non-Human Primates

The phenylalanine-metabolizing activity of SYN-PKU-2002 in healthy male cynomolgus monkeys was characterized by measuring the levels of phenylalanine (Phe) and its metabolites in plasma and urine following administration of SYN-PKU-2002 at dose levels ranging from 3.3×109 to 7.2×10¹¹ colony forming units (CFUs).

In this study, healthy male cynomolgus monkeys (2.8-4.5 kg) were fasted overnight. Prior to dosing, plasma samples (heparin) were taken to establish a baseline for unlabeled Phe and downstream metabolites. Animals were dosed, by oral gavage, consecutive solutions of 11 mL of peptone (5.5 g), SYN-PKU-2002 (doses ranging from 3.3×10⁹ to 7.2×10¹¹ CFU/dose), and 5 mL of 0.36 M sodium bicarbonate. Plasma was collected 0.5, 1, 2, 4, and 6 hours post dose and frozen until analysis. Plasma samples collected during the study arms were analyzed for the presence of Phe, phenylpyruvate (PP), trans-cinnamate (TCA), and hippurate (HA) using liquid chromatography-tandem mass spectrometry (LC-MS/MS). Urine was collected over the 6-hour post-dose period, the volume measured, and a 5-mL aliquot was frozen and stored until LC-MS/MS analysis.

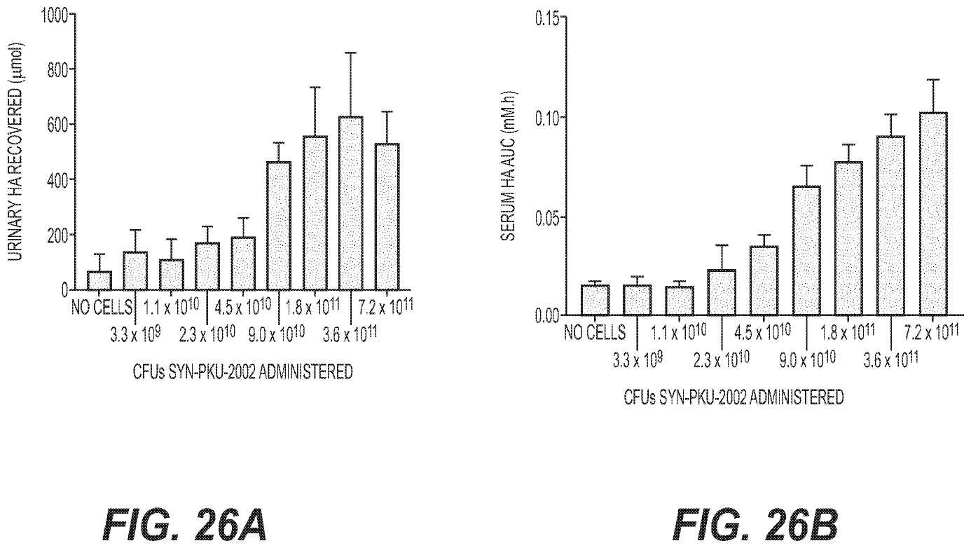

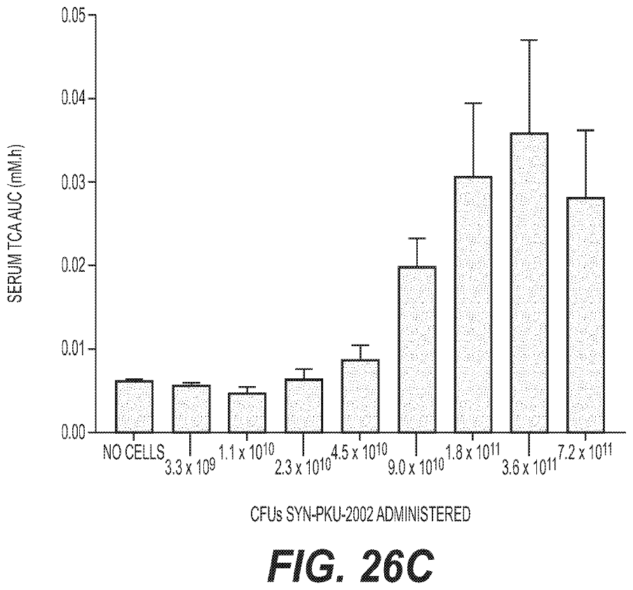

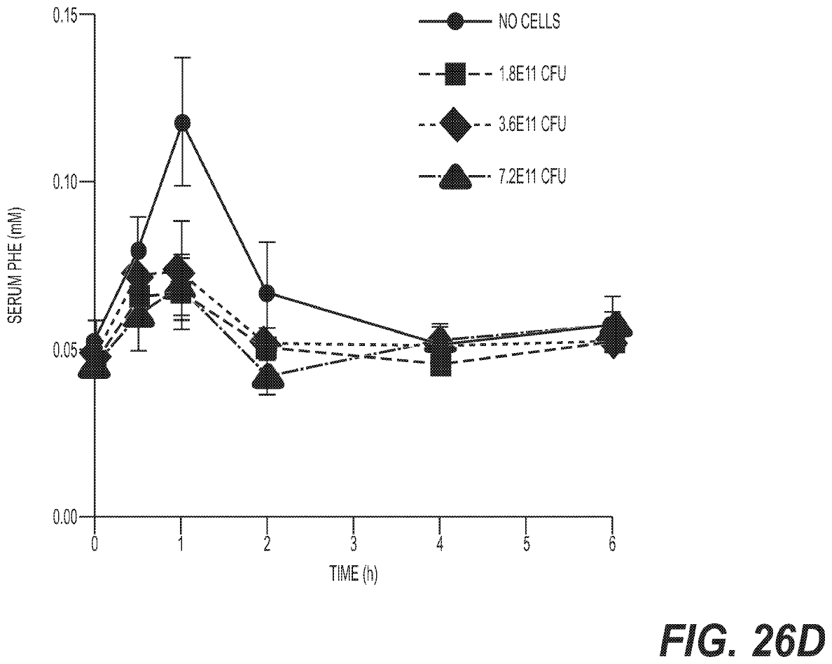

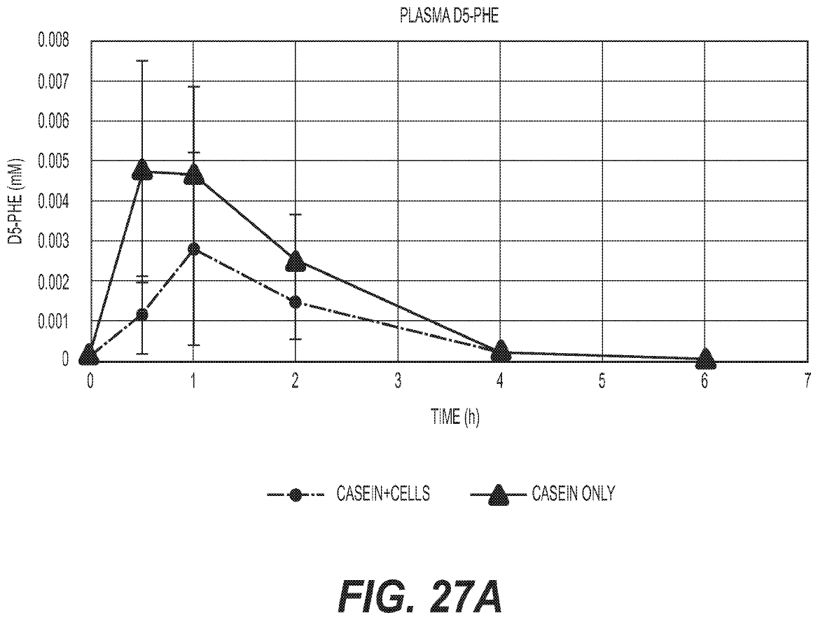

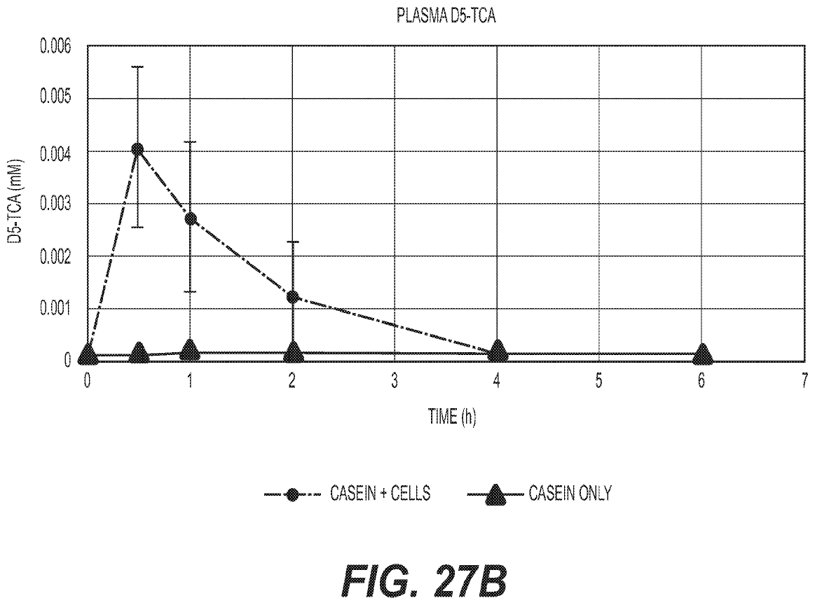

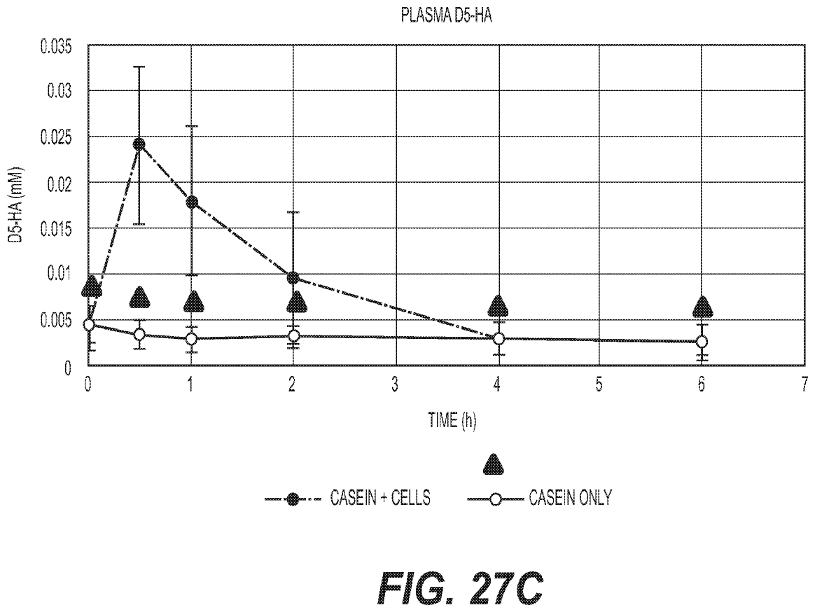

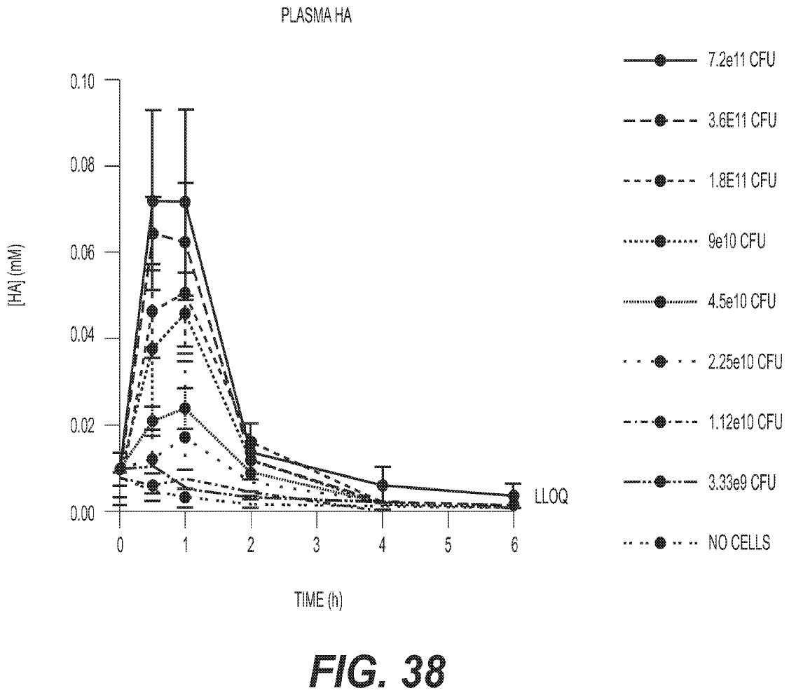

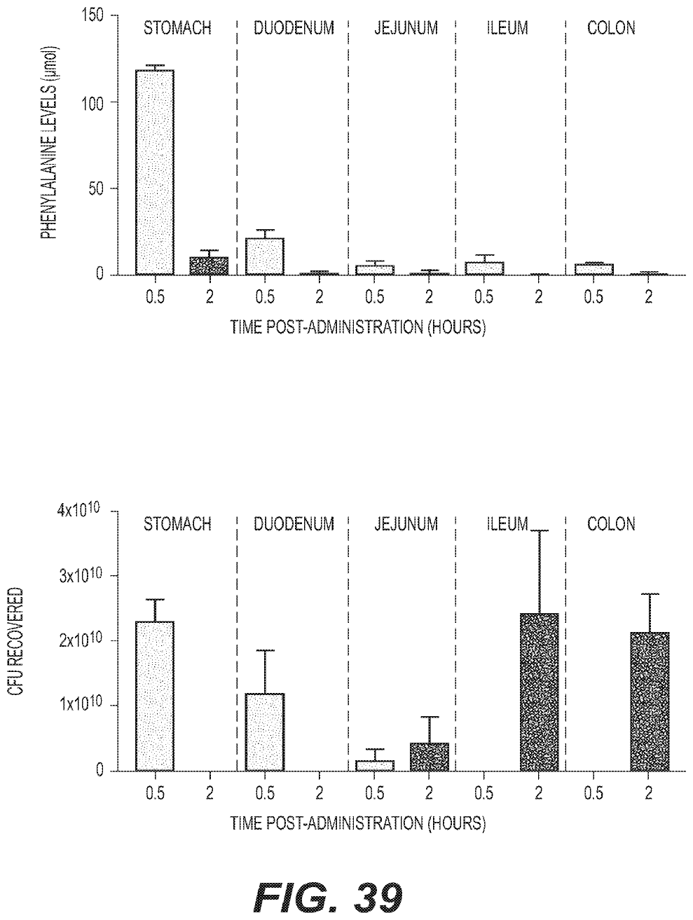

Administration of peptone to healthy monkeys resulted in a spike in plasma Phe concentrations peaking around 1 hour post-administration (see FIG. 26D). Oral dosing of SYN-PKU-2002 to healthy monkeys receiving 5.5 g of peptone resulted in a blunting of this Phe spike. Although not completely dose-proportional over the entire range, plasma concentrations of Phe were generally lower in the SYN-PKU-2002-dosed groups, particularly at or above the dose level of 9×10¹⁰ CFUs. Concurrent with decreases in Phe following SYN-PKU-2002 dosing, plasma concentrations of TCA (the direct product of phenylalanine ammonia lyase metabolism of Phe, see FIG. 26C) and HA (a metabolite of TCA, see FIG. 26B and FIG. 38) increased in a dose-dependent manner; however, there were no detectable plasma concentrations of PP (the direct product of L-amino acid deaminase metabolism of Phe) in monkeys treated with the highest dose (lower doses not measured). Hippurate is known to be rapidly excreted in urine and consistent with increased plasma TCA and HA concentrations, urinary HA excretion increased in a generally dose dependent manner (see FIG. 26A, no PP was detected in urine of the highest dose monkeys).

These studies demonstrate that SYN-PKU-2002 is able to metabolize Phe in male cynomolgus monkeys. PP was not detected in plasma and urine of monkeys treated with the highest dose. There was a dose-dependent increase in the plasma concentrations of the phenylalanine metabolites TCA and HA as well as dose-dependent increases in urinary HA excretion over 6 hours. This indicates that between the two Phe-degrading enzymes in SYN-PKU-2002, PAL is responsible for most of the Phe metabolism in the monkey, with little or no contribution from LAAD in this model. In addition, this study shows that HA and TCA could potentially serve as biomarkers of SYN-PKU-2002 activity in future clinical studies.

### Example 86: Gastrointestinal Tract Characterization of SYN-PKU-2001 in Non-Human Primates

Two studies were performed to characterize the phenylalanine-metabolizing activity and colonization of SYN-PKU-2001 within different segments of the gastrointestinal tract of healthy cynomolgus monkeys. In the first study, animals were separated into 2 groups of 3 females each, dosed with 5.5 grams of peptone, 5 mL of 0.36 M Sodium Bicarbonate, 25 mg/kg of D5-Phenylalanine, and either SYN-PKU-2001 or a blank control. Two hours after dosing, animals were euthanized and samples collected. In the second study, one group of 3 male monkeys were dosed with 5.5 grams of peptone, 5 mL of 0.36 M Sodium Bicarbonate, 25 mg/kg of D5-Phenylalanine, and SYN-PKU-2001, and animals were euthanized and samples collected 0.5 hours after dosage.

In both studies, animals were administered test articles (see table below) by a single oral gavage on Day 1. The capped bacteria tube was inverted 3 times before each dose administration. Dose formulations were administered by oral gavage using a disposable catheter attached to a plastic syringe. Following dosing, the gavage tube was rinsed with 5 mL of the animal drinking water, into the animal's stomach. Each animal was dosed with a clean gavage tube. The first day of dosing was designated as Day 1. The animals were temporarily restrained for dose administration and were not sedated.

### Test and Control Article Identification

Test and Control Article Identification

| | Test Article | Test Article | Test Article | | Test Article | Test Article |
|---|---|---|---|---|---|---|
| Identification | Peptone | Bacteria | D5-Phenylalanine | | Formulation | Sodium |
| | | | | | buffer | Bicarbonate |
| Batch ID | | SYN-PKU-2001 | | | | |
| | | 2917-C | | | | |
| Concentration | 500 g/L | 5.5 × 10¹⁰ | 20 | mg/mL | | 0.36M |
| | | CFU/mL | | | | |
| Physical | liquid | liquid | liquid | | | liquid |
| Description | | | | | | |
| Storage | Controlled Room | −80° C. | 4° | C. | 4° C. | Controlled Room |
| Conditions | Temperature | | | | | Temperature |

Formulation buffer and D5-Phenylalanine (20 mg/mL) were maintained at 4° C. until use, at which point, aliquots were removed and allowed to warm to room temperature for at least 30 minutes before dosing. Sodium Bicarbonate (0.36M) was stored and administered at ambient temperature.

Shown in the table below are the dose volumes and regimens used for each group of animals.

### Experimental Design

Experimental Design

| | | | Dose | |
|---|---|---|---|---|
| | No. | | Volume | |
| Group (Study No.) | Animals | Treatment | (mL) | Dose Regimen |
| Group 1 (Study No. 1) | 3 | Peptone | 11 | PO |
| | | Sodium | 5 | |
| | | Bicarbonate | | |
| | | D5-Phe | 1.3 | |
| | | Bacteria | 1.8 | |
| Group 2 (Study No. 1) | 3 | Peptone | 11 | |
| | | Sodium | 5 | |
| | | Bicarbonate | | |
| | | D5-Phe | 1.3 | |
| | | Formulation | 1.8 | |
| | | buffer | | |
| Group 1 (Study No. 3) | 3 | Peptone | 11 | PO |
| | | Sodium | 5 | |
| | | Bicarbonate | | |
| | | D5-Phe | 1.3 | |
| | | Bacteria | 1.8 | |

The dose of 5.5 grams of Peptone is based on a typical 60 kg individual consuming 100 g of protein per day and we wanted to simulate this so that we could measure Phe in the blood after feeding and metabolites of Phe in the urine. The oral dose of 25 mg/kg of D5-Phenylalanine was chosen because it gives measurable levels of D5-Hippurate in the urine of mice when dosed at this level.

In both studies, animals were fasted overnight, and weighed on day 1 before dosing. Blood samples (5 mL) were collected from the femoral vein using Heparin as the anticoagulant, and placed on crushed wet ice before centrifugation. Following centrifugation, the resultant plasma was separated, transferred into two aliquots, immediately frozen over dry ice and transferred to a freezer set to −80° C. Samples were shipped on dry ice to a separate facility for analysis.

Animals were euthanized (without sedation) 2 hours (Study 1) or 0.5 hours (Study 2) post dose via intravenous injection via tail vein, using a percutaneous catheter set-up, of a commercially available veterinary euthanasia solution.

### Terminal Procedures for Study

Terminal Procedures for Study

| | | Scheduled | Necropsy Procedures | | Histology | | |
|---|---|---|---|---|---|---|---|
| Study | No. of | Euthanasia | | Tissue | Organ | | |
| No. | Animals | Day^{a} | Necropsy | Collection | Weights | | Histopathology |
| 1 | 6 (F) | 1 | — | X | — | — | — |
| 2 | 3 (M) | | | | | | |

Tissues identified in the Tissue Collection and Preservation table were collected from all animals as indicated.

### Tissue Collection and Preservation

Tissue Collection and Preservation

| | | | Microscopic | |
|---|---|---|---|---|
| Tissue | Weigh | Collect | Evaluation | Comment |
| Large intestine, proximal | — | X | — | Contents, whole tissue (Studies 1 and 2) and mucus |
| colon | | | | scrappings (study 2 only). |
| Small intestine, duodenum | — | X | — | Contents and whole tissue in 30 cm segments (see |
| Small intestine, ileum | — | X | — | below). |
| Small intestine, jejunum | — | X | — | |
| Stomach | — | X | — | Contents and whole tissue (see below). |

X = Procedure to be conducted; — = Not applicable.

Following exsanguination, the stomach and colon were clamped. Additional clamps were used to section the small intestine at approximately 30 cm segments. 10 mL of cold sterile saline was injected into the stomach and massaged to loosen contents. The stomach was opened and contents drained into a labeled 50 mL conical tube and volume recorded. Approximately 1 mL of contents was transferred to the analysis facility to plate at time of collection. Remaining stomach contents were separated in 3 different aliquots: 1 mL, 1.6 mL (to which sponsor added glycerol before freezing) and remaining. The remaining stomach tissue (whole and collected into a labeled ziplock bag) was frozen on dry ice until shipped, with contents, via same-day courier (frozen on dry ice) to the analysis facility.

Each approximate 30 cm segment of the small intestine was injected with 15 mL of cold sterile saline and contents collected into a labeled 50 mL conical tube and volume recorded. Approximately 1 mL of contents from each segment was transferred to the analysis facility to plate at time of collection. Remaining contents were separated in 3 different aliquots: 1 mL, 1.6 mL (to which sponsor added glycerol before freezing) and remaining. Small intestine tissue sections (collected into labeled ziplock bags) were frozen on dry ice until shipped, with contents, via same-day courier (frozen on dry ice) to the analysis facility.

In the first study, no more than 20 mL of proximal colon contents were collected (undiluted) into a clean, pre-weighed glass bottle maintained on wet ice. Approximately 1 mL of contents were transferred to the analysis facility to plate at time of collection. Two different aliquots: 1 mL frozen on dry ice and 1.6 mL (to which sponsor added glycerol before freezing) were transferred to pre-identified tubes. The colon was then opened and the remaining content removed and transferred to the glass bottle (if less than 50 mL was previously collected). Tissue was grasped with forceps and gently rinsed in a basin containing sterile saline. The colon tissue was then scraped using a clean spatula to collect mucus into a labeled and pre-weighed glass bottle. The mucus sample was kept on wet ice until transferred to the analysis facility. The tissue was collected (whole) in a ziplock bag and frozen on dry ice until shipped via same-day courier (frozen on dry ice) to the analysis facility.

In the second study, 15 mL of cold sterile saline was injected into the proximal colon and the content was collected into a labeled 50 mL conical tube and the volume recorded. Approximately 1 mL of contents was transferred to the analysis facility to plate at time of collection. Remaining contents were separated into 3 different aliquots: 1 mL, 1.6 mL (to which sponsor added glycerol before freezing) and remaining. Large intestine tissue (collected into labeled ziplock bags) was frozen on dry ice until shipped, with contents, via same-day courier (frozen on dry ice) to the analysis facility at the address below.

The measured Phe levels and CFU counts for each part of the gastrointestinal tract are displayed in FIG. 38. Both 2 hours, and 0.5 hours after doseage, phenylalanine levels were found to be highest in the stomach and gradually decrease further down the gut. 2 hours after administration, less than 10% of phenylalanine was found in the stomach and duodenum. 2 hours post-dosage, SYN-PKU-2001 was found mainly in the ileum and proximal colon, where very little substrate is available. This suggests that SYN-PKU-2001 activity is limited by substrate availability and should thus be taken with food.

1.-73. (canceled)

74. An E. coli Nissle bacterium, wherein the bacterium comprises
(i) one or more gene(s) encoding a phenylalanine ammonia lyase (PAL) enzyme comprising an amino acid sequence having at least 90% identity to SEQ ID NO: 3, and
(ii) one or more non-native deletions in one or more phage genes in E. coli Nissle Phage 3, wherein the one or more phage queens are selected from the group consisting of ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

75. The bacterium of claim 74, wherein the non-native deletions comprise a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175.

76. The bacterium of claim 74, wherein the non-native deletions are: a complete deletion of ECOLIN_10110, ECOLIN_10115, ECOLIN 10120, ECOLIN_10125, ECOLIN_10130, ECOLIN_10135, ECOLIN_10140, ECOLIN 10145, ECOLIN_10150, ECOLIN_10160, ECOLIN_10165, ECOLIN_10170, and a partial deletion of ECOLIN_10175.

77. The bacterium of claim 76, wherein the deleted phage genes comprise SEQ ID NO: 130, or the deleted phage genes consist of SEQ ID NO: 130.

78. The bacterium of claim 74, wherein the bacterium further comprises an antibiotic resistance gene.

79. The bacterium of claim 74, further comprising one or more gene(s) encoding a phenylalanine transporter.

80. The bacterium of claim 79, wherein the phenylalanine transporter is PheP.

81. The bacterium of claim 79, further comprising one or more gene(s) encoding L-amino acid deaminase (LAAD).

82. The bacterium of claim 81, wherein:
the one or more genes encoding PAL are operably linked to a promoter;
the one or more genes encoding the phenylalanine transporter are operably linked to a promoter; and
the one or more genes encoding LAAD are operably linked to a promoter.

83. The bacterium of claim 82, wherein:
the one or more genes encoding PAL are operably linked to an IPTG-inducible promoter; and
the one or more genes encoding LAAD are operably linked to an arabinose-inducible promoter.

84. The bacterium of claim 82, wherein:
the one or more genes encoding PAL are operably linked to an IPTG-inducible promoter;
the one or more genes encoding the phenylalanine transporter are operably linked to an IPTG-inducible promoter; and
the one or more genes encoding LAAD are operably linked to an arabinose-inducible promoter.

85. The bacterium of claim 82, wherein:
the one or more genes encoding PAL are operably linked to an IPTG-inducible promoter;
the one or more genes encoding the phenylalanine transporter are operably linked to an IPTG-inducible promoter; and
the one or more genes encoding LAAD are operably linked to an arabinose-inducible promoter.

86. The bacterium of claim 82, wherein:
the one or more genes encoding PAL are operably linked to an FNR-responsive promoter;
the one or more genes encoding the phenylalanine transporter are operably linked to an FNR-responsive promoter; and
the one or more genes encoding LAAD are operably linked to an arabinose-inducible promoter.

87. The bacterium of claim 83, wherein:
the one or more of the genes encoding PAL are operably linked to an FNR-responsive promoter;
one or more additional genes encoding PAL comprise an amino acid sequence having at least 90% identity to SEQ ID NO: 3 and are operably linked to an IPTG-inducible promoter;
the one or more genes encoding the phenylalanine transporter are operably linked to an FNR-responsive promoter; and
the one or more genes encoding LAAD are operably linked to an arabinose-inducible promoter.

88. The bacterium of claim 82, wherein the bacterium comprises:
three copies of the gene encoding PAL comprise an amino acid sequence having at least 90% identity to SEQ ID NO: 3;
two additional copies of the gene encoding PAL comprise an amino acid sequence having at least 90% identity to SEQ ID NO: 3;
two copies of the gene encoding the phenylalanine transporter are non-native and derived from Escherichia coli;
one copy of the gene encoding LAAD is derived from Proteus mirabilis; and
the bacterium further comprises a mutation in one or more of ThyA or DapA to generate an auxotrophy.

89. The bacterium of claim 88, wherein:
the promoter operably linked to the three copies of the gene encoding PAL is inducible under anaerobic or low-oxygen conditions;
the promoter operably linked to the two additional copies of the gene encoding PAL is an IPTG-inducible promoter;
the promoter operably linked to the two copies of the non-native gene encoding the phenylalanine transporter derived from Escherichia coli is inducible under anaerobic or low-oxygen conditions;
the promoter operably linked to the one copy of the gene encoding LAAD derived from Proteus mirabilis is an arabinose-inducible promoter; and
the mutation in one or more of ThyA or DapA is a mutation in the DapA gene.

90. The bacterium of claim 89, wherein:
the promoter to which the three copies of the gene encoding PAL are operably linked to is an FNR-responsive promoter; and
the promoter to which the two copies of the non-native gene encoding the phenylalanine transporter derived from Escherichia coli are operably linked to is an FNR-responsive promoter.

91. The bacterium of claim 82, wherein the bacterium comprises:
four copies of the gene encoding PAL;
one copy of the gene encoding PheP phenylalanine transporter is non-native and derived from Escherichia coli;
one copy of the gene encoding LAAD is derived from Proteus mirabilis; and the bacterium further comprising a mutation in one or more of ThyA or DapA to generate an auxotrophy.

92. The bacterium of claim 91, wherein:
the promoter operably linked to the four copies of the gene encoding PAL is an IPTG-inducible promoter;
the promoter operably linked to the one copy of the non-native gene encoding the phenylalanine transporter derived from Escherichia coli is an IPTG-inducible promoter;
the promoter operably linked to the one copy of the gene encoding LAAD derived from Proteus mirabilis is an arabinose-inducible promoter; and
the mutation in one or more of ThyA or DapA is a mutation in the DapA gene.

93. A pharmaceutically acceptable composition comprising the bacterium of claim 74, and a pharmaceutically acceptable carrier.

94. The composition of claim 93 formulated for oral administration.
